(19) United States
(12) Patent Application Publication
(10) Pub. No.: US 2026/0328755 A1
(43) Pub. Date: Oct. 22, 2026

(54) METHODS, SYSTEMS, AND KITS FOR TREATMENT OF INFLAMMATORY DISEASES TARGETING TL1A

(71) Applicant: PROMETHEUS BIOSCIENCES, INC., San Diego, CA (US)

(72) Inventor: Fadi Towfic, La Jolla, CA (US)

(73) Assignee: PROMETHEUS BIOSCIENCES, INC., San Diego, CA (US)

(21) Appl. No.: 19/485,687

(22) PCT Filed: May 24, 2024
(86) PCT No.: PCT/US2024/030925

§ 371 Date: Nov. 19, 2025

Related U.S. Application Data
(60) Provisional application No. 63/504,540, filed on May 26, 2023.

(51) Int. Cl. G01N 33/53; C07K 16/28; G01N 33/68; G16B 25/10; G16H 10/40; G16H 10/60; G16H 20/10

(52) U.S. Cl. CPC G01N 33/5306; C07K 16/2875; G01N 33/6863; G01N 33/6893; G16B 25/10; G16H 10/40; G16H 10/60; G16H 20/10; C07K 2317/565; G01N 2333/525; G01N 2800/52; G01N 2800/7052; G01N 2800/7095

(57) ABSTRACT

Provided are methods, systems, and kits for selecting a subject for treatment with an inhibitor of Tumor necrosis factor-like cytokine 1A (TL1A) activity or expression based on the ratio between the TL1A isoforms.

### CROSS-REFERENCE TO RELATED APPLICATIONS

This application claims the benefit of U.S. Provisional Patent Application Ser. No. 63/504,540 filed May 26, 2023, the entire contents of which are incorporated by reference herein.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on May 23, 2024, is named 25743-WO-PCT_SL.xml and is 1,346,000 bytes in size.

### 1. BACKGROUND

Inflammatory disease, fibrostenotic disease, and fibrotic disease pose a significant health burden worldwide due to the vast number of individuals affected and heterogeneous disease pathogenesis and varied clinical manifestations. One such disease is inflammatory bowel disease (IBD), which has two common forms, Crohn's disease (CD) and ulcerative colitis (UC). IBD is the chronic, relapsing inflammatory disorders of the gastrointestinal tract. Incidences of IBD are prevalent, affecting nearly three million individuals in the United States alone.

Few treatment options are available to patients that suffer from inflammatory disease, fibrostenotic disease, and fibrotic disease. Existing anti-inflammatory therapy such as steroids and tumor necrosis factor (TNF) inhibitors are typically used as a first line treatment for treating IBD. Unfortunately, a significant number of patients experience a lack of response or a loss of response to existing anti-inflammatory therapies, especially TNF inhibitors. While the patient is treated with an anti-inflammatory therapy that is ineffective, the disease worsens. Surgery, in the form of structureplasty (reshaping of the intestine) or resection (removal of the intestine), is the only treatment option for patients that do not respond to first line therapies. Surgical treatments for IBD are invasive, causing post-operative risks for an estimated third of patients undergoing surgery, such as anastomotic leak, infection, and bleeding.

The pathogenesis of inflammatory disease, fibrostenotic disease, and fibrotic disease, like IBD, is thought to involve an uncontrolled immune response that may be triggered by certain environmental factors in a genetically susceptible individual. The heterogeneity of disease pathogenesis and clinical course, combined with the variable response to treatment and its associated side effects, suggests a personalized medicine approach to treating these diseases is the best treatment strategy. Yet there are very few personalized therapies available to patients. Accordingly, there is a need to identify targeted therapeutic approaches for the treatment of inflammatory disease, fibrostenotic disease, and fibrotic disease and subclinical phenotypes thereof, and an even greater need to develop reliable methodology to identifying patients who may respond to any given therapeutic approach. The needed methodologies would also identify subjects not yet diagnosed who are at risk of developing the disease, for which preventative interventions could be prescribed to reduce the growing health burden.

### 2. SUMMARY

The TL1A isoform ratios, proxy markers, and CDx models based on them described herein are associated (individually or together) with (i) an increase in a level of TNFSF15 (TL1A) protein expression in a sample obtained from a subject or patient, as compared to a reference level of TNFSF15 (TL1A) protein expression (e.g., derived from a normal individual), and/or (ii) an increase of a positive therapeutic response in IBD patients to a treatment with the TL1A inhibitor as compared to the reference level of response in patients not selected by the TL1A isoform ratios, proxy markers, and CDx models. More specifically, the TL1A isoform ratios, proxy markers, and CDx models based on them described herein are associated (individually or together) with (i) alone; (ii) alone; or (i) and (ii) together, wherein (i) and (ii) corresponds to the (i) and (ii) numbered items in the previous clause of this paragraph. Accordingly and as an example, the TL1A isoform ratios, proxy markers, and CDx models based on them described herein are associated with an increase in a level of TNFSF15 (TL1A) protein expression in a sample obtained from a subject or patient, as compared to a reference level of TNFSF15 (TL1A) protein expression (e.g., derived from a normal individual). Additionally, the TL1A isoform ratios, proxy markers, and CDx models based on them described herein are associated) with an increase of a positive therapeutic response in IBD patients to a treatment with the inhibitor of TL1A activity or expression, as compared to the reference level of response in patients not selected by the TL1A isoform ratios, proxy markers, and CDx models. The patient may be diagnosed with IBD, CD, or both. The subject may be suspected of having IBD, CD, or both.

In one aspect, provided herein is a method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a ratio between TL1A short isoform and TL1A long isoform (“TL1A isoform ratio”), a proxy marker having a statistically significant correlation with the TL1A isoform ratio, or a combination thereof.

In another aspect, provided herein is a method of treating an inflammatory, a fibrotic, or a fibrostenotic disease or condition in a subject, the method comprising: (a) determining whether the subject with an inflammatory, a fibrotic, or a fibrostenotic disease or condition is suitable for treatment with an inhibitor of TL1A activity or expression by: (i) obtaining or having obtained a sample from the subject; (ii) subjecting the sample to an assay adapted to determine a ratio between TL1Ashort isoform and TL1A long isoform (“TL1A isoform ratio”), a proxy marker having a statistically significant correlation with the TL1A isoform ratio, or a combination thereof; (iii) determining the subject is suitable for treatment with an inhibitor of TL1A activity or expression based on the TL1A isoform ratio or the proxy marker; and (b) treating the subject by administering a therapeutically effective amount of the inhibitor of TL1A activity or expression to the subject.

In a further aspect, provided herein is a method of determining a ratio between TL1A short isoform and TL1A long isoform (“TL1A isoform ratio”) or a proxy marker having a statistically significant correlation with the TL1A isoform ratio for a subject with an inflammatory, a fibrotic, or a fibrostenotic disease or condition, the method comprising: (a) obtaining or having obtained a sample from the subject; (b) subjecting the sample to an assay adapted to detect an expression level of the TL1Ashort isoform and an expression level of the TL1A long isoform; and (c) determining a ratio TL1A isoform ratio, a proxy marker having a statistically significant correlation with the TL1A isoform ratio, or a combination thereof, wherein whether the subject is suitable for treatment with an inhibitor of TL1A activity or expression is indicated based on the TL1A isoform ratio or the proxy marker.

In yet another aspect, provided herein is a method of selecting a subject with an inflammatory, a fibrotic, or a fibrostenotic disease or condition for treatment with an inhibitor of TL1A activity or expression, the method comprising: (a) obtaining or having obtained a sample from the subject; (b) subjecting the sample to an assay adapted to detect an expression level of the TL1Ashort isoform and an expression level of the TL1A long isoform; (c) determining a ratio between TL1Ashort isoform and TL1A long isoform (“TL1A isoform ratio”), a proxy marker having a statistically significant correlation with the TL1A isoform ratio, or a combination thereof; and (d) selecting the subject for treatment with the inhibitor of TL1A activity or expression based on the TL1A isoform ratio or the proxy marker.

In some embodiments, the method further comprises preparing nucleic acids from the sample.

In some embodiments, the correlation is Pearson correlation or Spearman correlation. In some embodiments, the correlation has a correlation coefficient with an absolute value of at least about 0.30, at least about 0.31, at least about 0.32, at least about 0.33, at least about 0.34, at least about 0.35, at least about 0.36, at least about 0.37, at least about 0.38, at least about 0.39, at least about 0.40, at least about 0.41, at least about 0.42, at least about 0.43, at least about 0.44, at least about 0.45, at least about 0.46, at least about 0.47, at least about 0.48, at least about 0.49, at least about 0.50, at least about 0.51, at least about 0.52, at least about 0.53, at least about 0.54, at least about 0.55, at least about 0.56, at least about 0.57, at least about 0.58, at least about 0.59, at least about 0.60, at least about 0.61, at least about 0.62, at least about 0.63, at least about 0.64, at least about 0.65, at least about 0.66, at least about 0.67, at least about 0.68, at least about 0.69, at least about 0.70, at least about 0.71, at least about 0.72, at least about 0.73, at least about 0.74, at least about 0.75, at least about 0.76, at least about 0.77, at least about 0.78, at least about 0.79, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or 1.

In one aspect, provided herein is a method of identifying a proxy marker for a ratio between TL1Ashort isoform and TL1A long isoform (“TL1A isoform ratio”), the method comprising: (a) determine the TL1A isoform ratio in a population of subjects; (b) determining a marker other than the TL1A isoform ratio in the population of subjects; (c) determining the correlation between the marker in (b) and the TL1A isoform ratio in the population; and (d) identifying the marker from (b) as a proxy marker for the TL1A isoform ratio if the marker has a statistically significant correlation with the TL1A isoform ratio, wherein optionally the proxy marker relative to a cutoff is indicative that the subject is suitable for treatment with an inhibitor of TL1A activity or expression for treatment of the inflammatory, fibrotic, or fibrostenotic disease or condition. In some embodiments, the proxy marker is identified by the method of described in this paragraph.

In one aspect, provided herein is a computer-implemented method of determining a ratio between TL1Ashort isoform and TL1A long isoform (“TL1A isoform ratio”) for a subject, the method comprising: (a) receiving TL1A expression data obtained from a sample from the subject with an inflammatory, a fibrotic, or a fibrostenotic disease or condition; (b) analyzing the TL1A expression data with a statistical algorithm configured to produce the TL1A isoform ratio; and (d) applying a cutoff to the TL1A isoform ratio, wherein the TL1A isoform ratio relative to the cutoff is indicative that the subject is suitable for treatment with an inhibitor of TL1A activity or expression for treatment of the inflammatory, fibrotic, or fibrostenotic disease or condition.

In another aspect, provided herein is a computer-implemented method of determining a ratio between TL1Ashort isoform and TL1A long isoform (“TL1A isoform ratio”) for a subject, the method comprising: (a) obtaining expression data in a sample from the subject; (b) determining expression of TL1Ashort isoform and TL1A long isoform; (c) calculating the TL1A isoform ratio; and (d) applying a cutoff to the TL1A isoform ratio, wherein the TL1A isoform ratio relative to the cutoff is indicative that the subject is suitable for treatment with an inhibitor of TL1A activity or expression for treatment of the inflammatory, fibrotic, or fibrostenotic disease or condition.

In some embodiments of the methods provided herein, based on the TL1A isoform ratio is based on TL1A isoform ratio above a cutoff.

In some embodiments of the methods provided herein, the TL1A isoform ratio relative to the cutoff is the TL1A isoform ratio above the cutoff.

In some embodiments of the methods provided herein, the proxy marker comprises rs6478109.

In some embodiments of the methods provided herein, based on the proxy marker is (i) based on the proxy marker above a cutoff if the proxy marker and the TL1A isoform ratio has a positive correlation (such proxy marker as “positive proxy marker”); or (ii) based on the proxy marker below a cutoff if the proxy marker and the TL1A isoform ratio has a negative correlation (such proxy marker as “inverse proxy marker”).

In some embodiments of the methods provided herein, the positive proxy marker is identified by a method of identifying a proxy marker for a ratio between TL1Ashort isoform and TL1A long isoform (“TL1A isoform ratio”), the method comprising: (a) determine the TL1A isoform ratio in a population of subjects; (b) determining a marker other than the TL1A isoform ratio in the population of subjects; (c) determining the correlation between the marker in (b) and the TL1A isoform ratio in the population; and (d) identifying the marker from (b) as a proxy marker for the TL1A isoform ratio if the marker has a statistically significant positive correlation with the TL1A isoform ratio, wherein optionally the proxy marker relative to a cutoff is indicative that the subject is suitable for treatment with an inhibitor of TL1A activity or expression for treatment of the inflammatory, fibrotic, or fibrostenotic disease or condition.

In some embodiments of the methods provided herein, TL1A isoform ratio or a positive proxy marker above a cutoff is determined if an inverse-proxy marker is below an alternative cutoff, wherein the inverse proxy marker has a negative correlation with the TL1A isoform ratio. In some embodiments of the methods provided herein, inverse proxy marker comprises the ratio of TL1A long isoform to the TL1Ashort isoform (TL1Along/TL1Ashort).

In some embodiments of the methods provided herein, the inverse proxy marker is identified by a method of identifying a proxy marker for a ratio between TL1Ashort isoform and TL1A long isoform (“TL1A isoform ratio”), the method comprising: (a) determine the TL1A isoform ratio in a population of subjects; (b) determining a marker other than the TL1A isoform ratio in the population of subjects; (c) determining the correlation between the marker in (b) and the TL1A isoform ratio in the population; and (d) identifying the marker from (b) as a proxy marker for the TL1A isoform ratio if the marker has a statistically significant negative correlation with the TL1A isoform ratio, wherein optionally the proxy marker relative to a cutoff is indicative that the subject is suitable for treatment with an inhibitor of TL1A activity or expression for treatment of the inflammatory, fibrotic, or fibrostenotic disease or condition.

In some embodiments of the methods provided herein, the TL1A isoform ratio above a cutoff is determined if the ratio of TL1A long isoform to the TL1Ashort isoform is below an alternative cutoff.

In some embodiments of the methods provided herein, the cutoff ranges from about 0.090 to about 0.110. In some embodiments, the cutoff is about 0.097. In some embodiments, the cutoff in this paragraph is for selecting CD patients.

In some embodiments of the methods provided herein, the cutoff ranges from about 0.080 to about 0.10. In some embodiments the cutoff is about 0.085. In some embodiments, the cutoff in this paragraph is for selecting UC patients.

In some embodiments of the methods provided herein, the cutoff is (i) the TL1A isoform ratio or proxy marker level in a heathy subject without the inflammatory, fibrotic, or fibrostenotic disease or condition or (it) the TL1A isoform ratio or proxy marker level in a tissue not afflicted by the inflammatory, fibrotic, or fibrostenotic disease or condition.

In some embodiments of the methods provided herein, the TL1A isoform ratio is calculated as, TL1Ashort/TL1Along, ln(TL1Ashort/TL1Along), or ln(TL1Ashort/TL1Along+n), wherein TL1Ashort is an expression level of the TL1Ashort isoform, TL1Along is an expression level of the TL1A long isoform, and n is a positive number. In some embodiments, n is 1.

In some embodiments of the methods provided herein, the expression level of the TL1A short isoform is the level of gene expression of the TL1Ashort isoform and the expression level of the TL1A long isoform is the level of gene expression of the TL1A long isoform. In some embodiments of the methods provided herein, the expression level of the TL1Ashort isoform is the level of protein expression of the TL1Ashort isoform and the expression level of the TL1A long isoform is the level of protein expression of the TL1A long isoform. In some embodiments of the methods provided herein, the expression level of the TL1Ashort isoform is the level of gene expression of the TL1Ashort isoform and the expression level of the TL1A long isoform is the level of protein expression of the TL1A long isoform. In some embodiments of the methods provided herein, the expression level of the TL1Ashort isoform is the level of protein expression of the TL1Ashort isoform and the expression level of the TL1A long isoform is the level of gene expression of the TL1A long isoform.

In some embodiments of the methods provided herein, the TL1A isoform ratio or the proxy marker is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%, In some embodiments, the TL1A isoform ratio or the proxy marker is predictive of a positive therapeutic response in the subject to a treatment with the inhibitor of TL1A activity or expression with a specificity of at least about 50%, 55%, 60%, 65%, 70%, 75%, 80%, 85%, 90%, or 95%, In some embodiments, the TL1A isoform ratio or the proxy marker is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%, In some embodiments, the TL1A isoform ratio or the proxy marker is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%, In some embodiments, the TL1A isoform ratio or the proxy marker is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%, In some embodiments, the TL1A isoform ratio or the proxy marker is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%.

In some embodiments of the methods provided herein, the cutoff is such that the TL1A isoform ratio or the proxy marker above the cutoff is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%, In some embodiments, the cutoff is such that the TL1A isoform ratio or the proxy marker above the cutoff is predictive of a positive therapeutic response in the subject to a treatment with the inhibitor of TL1A activity or expression with a specificity of at least about 50%, 55%, 60%, 65%, 70%, 75%, 80%, 85%, 90%, or 95%, In some embodiments, the cutoff is such that the TL1A isoform ratio or the proxy marker above the cutoff is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%, In some embodiments, the cutoff is such that the TL1A isoform ratio or the proxy marker above the cutoff is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%, In some embodiments, the cutoff is such that the TL1A isoform ratio or the proxy marker above the cutoff is predictive of an increase of one or more IBD enriched cell types with a positive rate of at least about 10%, 15%, 20%, 25%, 30%, 35%, 40%, 45%, 50%, 55%, 60%, 65%, 70%, or 75%, In some embodiments, the cutoff is such that the TL1A isoform ratio or the proxy marker above the cutoff is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%.

In some embodiments of the methods provided herein, the TL1A isoform ratio is TL1A isoform ratio in a diseased tissue afflicted by the inflammatory, fibrotic, or fibrostenotic disease or condition. In some embodiments, the diseased tissue is selected from the group consisting of ileum, colon, rectum, or small intestine.

In some embodiments of the methods provided herein, the TL1A isoform ratio is TL1A isoform ratio in a cell contributory to the inflammatory, fibrotic, or fibrostenotic disease or condition. In some embodiments, the cell is selected from the group consisting of a Goblet cell, a pericyte, a smooth muscle cell, an enterocyte, a colonocyte, a monocyte-derived dendritic cells (moDC), a resident macrophage, or a colon transit-amplifying (TA) cell.

In some embodiments of the methods provided herein, the TL1A isoform ratio is TL1A isoform ratio in blood of the subject.

In one aspect, provided herein is a computer-implemented system comprising at least one processor and instructions executable by the at least one processor to provide an application configured to determine a ratio between TL1Ashort isoform and TL1A long isoform (“TL1A isoform ratio”) for a subject by performing operations comprising: (a) receiving expression data of TL1Ashort isoform and TL1A long isoform obtained from a sample from the subject with an inflammatory, a fibrotic, or a fibrostenotic disease or condition; (b) applying an algorithm to the expression data to produce the TL1A isoform ratio; and (d) applying a cutoff to the TL1A isoform ratio, wherein the TL1A isoform ratio relative to the cutoff is indicative that the subject is suitable for treatment with an inhibitor of TL1A activity or expression.

In another aspect, provided herein is a computer-implemented system comprising at least one processor and instructions executable by the at least one processor to provide an application configured to determine a ratio between TL1Ashort isoform and TL1A long isoform (“TL1A isoform ratio”) for a subject by performing operations comprising: (a) receiving expression data of TL1Ashort isoform and TL1A long isoform obtained from a sample from the subject with an inflammatory, a fibrotic, or a fibrostenotic disease or condition; and (b) calculating TL1A isoform ratio by utilizing one or more algorithms, wherein the TL1A isoform ratio relative to the cutoff is indicative that the subject is suitable for treatment with an inhibitor of TL1A activity or expression.

In some embodiments of the computer-implemented system provided herein, the TL1A isoform ratio relative to the cutoff is the TL1A isoform ratio above the cutoff.

In some embodiments of the computer-implemented system provided herein, the cutoff ranges from about 0.090 to 0.110. In some embodiments of the computer-implemented system provided herein, the cutoff is about 0.097. In some embodiments of the computer-implemented system provided in this paragraph, the cutoff is for selecting CD patients.

In some embodiments of the computer-implemented system provided herein, the cutoff ranges from about 0.080 to 0.10. In some embodiments of the computer-implemented system provided herein, the cutoff is about 0.085. In some embodiments of the computer-implemented system provided in this paragraph, the cutoff is for selecting UC patients.

In some embodiments of the computer-implemented system provided herein, the cutoff is (1) the TL1A isoform ratio or proxy marker level in a heathy subject without the inflammatory, fibrotic, or fibrostenotic disease or condition or (ii) the TL1A isoform ratio or proxy marker level in a tissue not afflicted by the inflammatory, fibrotic, or fibrostenotic disease or condition.

In some embodiments of the computer-implemented system provided herein, the TL1A isoform ratio is calculated as, TL1Ashort/TL1Along, ln(TL1Ashort/TL1Along), or ln(TL1Ashort/TL1Along+n), wherein TL1Ashort is an expression level of the TL1Ashort isoform, TL1Along is an expression level of the TL1A long isoform, and n is a positive number. In some embodiments of the computer-implemented system provided herein, n is 1.

In some embodiments of the computer-implemented system provided herein, the expression level of the TL1Ashort isoform is the level of gene expression of the TL1Ashort isoform and the expression level of the TL1A long isoform is the level of gene expression of the TL1A long isoform. In some embodiments of the computer-implemented system provided herein, the expression level of the TL1Ashort isoform is the level of protein expression of the TL1Ashort isoform and the expression level of the TL1A long isoform is the level of protein expression of the TL1A long isoform. In some embodiments of the computer-implemented system provided herein, the expression level of the TL1Ashort isoform is the level of gene expression of the TL1Ashort isoform and the expression level of the TL1A long isoform is the level of protein expression of the TL1A long isoform. In some embodiments of the computer-implemented system provided herein, the expression level of the TL1Ashort isoform is the level of protein expression of the TL1Ashort isoform and the expression level of the TL1A long isoform is the level of gene expression of the TL1A long isoform.

In some embodiments of the methods or the systems provided herein, the statistically significant correlation is a correlation with p value less than 0.1, 0.09, 0.08, 0.07, 0.06, 0.05, 0.04, 0.03, 0.02, or 0.01.

In some embodiments of the methods or the systems provided herein, the computer-implemented system of any one of claims 48 to 61, wherein the subject has been treated with an advanced IBD therapy prior to the treatment with the inhibitor of TL1A activity or expression. In some embodiments of the methods or the systems provided herein, the subject has not been treated with an advanced IBD therapy prior to the treatment with the inhibitor of TL1A activity or expression. In some embodiments of the methods or the systems provided herein, the advanced IBD therapy comprises one or more selected from the group consisting of a biologic therapeutic agent for IBD, an S1P1 modulator, or a JAK inhibitor. In some embodiments of the methods or the systems provided herein, the biologic therapeutic agent for IBD comprises an anti-TNFα antibody, an anti-IL23 antibody, or an anti-integrin α4β7 antibody.

In some embodiments of the methods or the systems provided herein, the inhibitor of TL1A activity or expression is an antibody or antigen binding fragment thereof that binds to TL1A (anti-TL1A antibody or antigen binding fragment), wherein the anti-TL1A antibody or antigen binding fragment comprises a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and a light chain variable region comprising: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15.

In some embodiments of the methods or the systems provided herein, the inhibitor of TL1A activity or expression is an anti-TL1A antibody or antigen binding fragment, wherein the anti-TL1A antibody or antigen binding fragment comprises a heavy chain variable domain comprising an amino acid sequence at least about 90% identical to any one of SEQ ID NOS: 101-135, or 310-302, and a light chain variable domain comprising an amino acid sequence at least about 90% identical to any one of SEQ ID NOS: 201-206 or 303. In some embodiments of the methods or the systems provided herein, the heavy chain variable domain comprises an amino acid sequence at least about 91%, 92%, 93%, 94%, 95%, 96%, 97%, 98%, 99%, or 100% identical to any one of SEQ ID NOS: 101-135, or 310-302. In some embodiments of the methods or the systems provided herein, the light chain variable domain comprises an amino acid sequence at least about 91%, 92%, 93%, 94%, 95%, 96%, 97%, 98%, 99%, or 100% identical to any one of SEQ ID NOS: 201-206 or 303.

In some embodiments of the methods or the systems provided herein, the inhibitor of TL1A activity or expression is an anti-TL1A antibody or antigen binding fragment, wherein the anti-TL1A antibody or antigen binding fragment comprises: (a) a heavy chain variable framework region comprising a human IGHV1-46*02 framework or a modified human IGHV1-46*02 framework; and (b) a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework. In some embodiments of the methods or the systems provided herein, the amino acid modification of the less than 14 amino acid modifications comprises: (a) the amino acid modification is at position 47 in the heavy chain variable region, and the amino acid at position 47 is R, N, D, C, Q, E, G, H, I, L, K, M, F, P, S, T, W, Y, or V; (b) the amino acid modification is at position 45 in the heavy chain variable region, and the amino acid at position 45 is A, N, D, C, Q, E, G, H, I, L, K, M, F, P, S, T, W, Y, or V; (c) the amino acid modification is at position 55 in the heavy chain variable region, and the amino acid at position 55 is A, R, N, D, C, Q, E, G, H, I, L, K, F, P, S, T, W, Y, or V; (d) the amino acid modification is at position 78 in the heavy chain variable region, and the amino acid at position 78 is A, R, N, D, C, Q, E, G, H, I, L, K, M, F, P, S, T, W, or Y; (e) the amino acid modification is at position 80 in the heavy chain variable region, and the amino acid at position 80 is A, R, N, D, C, Q, E, G, H, I, L, K, F, P, S, T, W, Y, or V; (f) the amino acid modification is at position 82 in the heavy chain variable region, and the amino acid at position 82 is A, N, D, C, Q, E, G, H, I, L, K, M, F, P, S, T, W, Y, or V; (g) the amino acid modification is at position 89 in the heavy chain variable region, and the amino acid at position 89 is A, R, N, D, C, Q, E, G, H, I, L, K, M, F, P, S, T, W, or Y; or (h) the amino acid modification is at position 91 in the heavy chain variable region, and the amino acid at position 91 is A, R, N, D, C, Q, E, G, H, I, L, K, F, P, S, T, W, Y, or V: or a combination of two or more modifications selected from (a) to (h). In some embodiments of the methods or the systems provided herein, the amino acid modification of the less than 14 amino acid modifications comprises: A47R, R45K, M551, V78A, M80I, R82T, V89A, M91L in the heavy chain variable region, per Aho or Kabat numbering. In some embodiments of the methods or the systems provided herein, the amino acid modification of the less than 14 amino acid modifications comprises: (a) a modification at amino acid position 54 in the light chain variable region; and/or (b) a modification at amino acid position 55 in the light chain variable region; per Aho or Kabat numbering. In some embodiments of the methods or the systems provided herein, the amino acid modification of the less than 14 amino acid modifications comprises: (a) the amino acid modification is at position 54 of the light chain variable region, and the amino acid at position 54 is A, R. N, D, C, Q, E, G, H, I, K, M, F, P, S, T, W, Y, or V; and/or (b) the amino acid modification is at position 55 of the light chain variable region, and the amino acid at position 55 is A, R, N, D, C, Q, E, G, H, I, K, M, F, P, S, T, W, Y, or V. In some embodiments of the methods or the systems provided herein, the amino acid modification of the less than 14 amino acid modifications comprises L54P and/or L55 W in the light chain variable region, per Aho or Kabat numbering.

In some embodiments of the methods or the systems provided herein, the inhibitor of TL1A activity or expression is an antibody or antigen binding fragment thereof that binds to TL1A and comprises: a heavy chain variable region comprising SEQ ID NO: 301 XIVQLVQSGAEVKKPGASVKVSCKAS[HCDR1]WVX2QX3PGQGLEWX4G[HCDR2]RX5 TX6TX7DTSTSTX8YX9ELSSLRSEDTAVYYCAR[HCDR3]WGQGTTVTVSS, and a light chain variable region comprising SEQ ID NO: 303 EIVLTQSPGTLSLSPGERATLSC[LCDR1]WYQQKPGQAPRX10XIIIY [LCDR2]GIPDRFSG SGSGTDFTLTISRLEPEDFAVYYC[LCDR3]FGGGTKLEIK, wherein each of XI-X11 is independently selected from A, R, N, D, C, Q, E, G, H, I, L, K, M, F, P, S, T, W, Y, or V. In some embodiments of the methods or the systems provided herein, the inhibitor of TL1A activity or expression is an antibody or antigen binding fragment thereof that binds to TL1A and comprises: a heavy chain variable region comprising SEQ ID NO: 302 XIVQLVQSGAEVKKPGASVKVSCKAS[HCDR1]WVX2QX3PGQGLEWX4G[HCDR2]RX5 TX6TX7DTSTSTX8YX9ELSSLRSEDTAVYYC[HCDR3]JWGQGTTVTVSS, and a light chain variable region comprising SEQ ID NO: 303 EIVLTQSPGTLSLSPGERATLSC[LCDR1]WYQQKPGQAPRX10X11IY [LCDR2]GIPDRFSG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V. In some embodiments of the methods or the systems provided herein, (a) X1 is Q or E; (b) X2 is R or K (c) X3 is A or R; (d) X4 is M or I; (e) X5 is V or A; (f) X6 is M or I; (g) X7 is R or T; (h) X8 is V or A: (i) X9 is M or L (j) X10 is L or P; (k) X11 is L or W; or (1) X1-X11 are any combination of (a) to (k).

In some embodiments of the methods or the systems provided herein, the antibody or antigen binding fragment comprises a heavy chain CDR1 as set forth by SEQ ID NO: 1, a heavy chain CDR2 as set forth by any one of SEQ ID NOS: 2-5, a heavy chain CDR3 as set forth by any one of SEQ ID NOS: 6-9, a light chain CDR1 as set forth by SEQ ID NO: 10, a light chain CDR2 as set forth by SEQ ID NO: 11, and a light chain CDR3 as set forth by any one of SEQ ID NOS: 12-15.

In some embodiments of the methods or the systems provided herein, the antibody or antigen binding fragment comprises a heavy chain framework (FR) 1 as set forth by SEQ ID NO: 304, a heavy chain FR2 as set forth by SEQ ID NO: 305 or SEQ ID NO: 313, a heavy chain FR3 as set forth by any one of SEQ ID NOS: 306, 307, 314, or 315, a heavy chain FR4 as set forth by SEQ ID NO: 308, a light chain FR1 as set forth by SEQ ID NO: 309, a light chain FR2 as set forth by SEQ ID NO: 310, a light chain FR3 as set forth by SEQ ID NO: 311, or a light chain FR4 as set forth by SEQ ID NO: 312, or a combination thereof.

In some embodiments of the methods or the systems provided herein, the antibody or antigen binding fragment comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (1) 327Q or 327T, (j) 329A, 329G, 329Y, or 329R (k) 331S, (1) 236F or 236R, (m) 238A, 238E, 238G, 238H, 238I, 238V, 238 W, or 238Y. (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K. (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376 W, or 376Y, (II) 380D, (mm) 382D or 382P, (nn) 385P, (00) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L23SE, and P33IS, (zz) L234A, L235E, and G237A, (aaa), L234A, L235E, G237A, and P331S (bbb) L234A, L235A, G237A. P238S, H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of two or more selected from (a)-(uu), per Kabat numbering.

In some embodiments of the methods or the systems provided herein, the antibody or antigen binding fragment comprises a human IgG4 Fc region.

In some embodiments of the methods or the systems provided herein, the antibody or antigen binding fragment comprises a Fc region comprising a sequence at least about 90%, 91%, 92%, 93%, 94%, 95%, 96%, 97%, 98%, 99%, or 100% identical to any one of SEQ ID NOS: 320-362.

In some embodiments of the methods or the systems provided herein, the antibody of antigen binding fragment comprises a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1.

In some embodiments of the methods or the systems provided herein, the antibody or antigen binding fragment comprises a Fc region and wherein the Fc comprises the human IgG1 comprises SEQ ID NO: 320.

In some embodiments of the methods or the systems provided herein, the antibody or antigen binding fragment comprises a Fc region and wherein the ADCC function of the Fc region comprising reduced ADCC is at least about 50% reduced as compared to human IgG1.

In some embodiments of the methods or the systems provided herein, the antibody or antigen binding fragment comprises a Fc region and wherein the CDC function of the Fc region comprising reduced CDC is at least about 50% reduced as compared to human IgG1.

In some embodiments of the methods or the systems provided herein, the antibody or antigen binding fragment comprises a Fc region and wherein the Fc comprises (i) a human IgG4 Fc region or (ii) a human IgG4 Fc region comprising (a) S228P, (b) S228P and L235E, or (c) S228P, F234A, and L235A, per Kabat numbering.

In some embodiments of the methods or the systems provided herein, the antibody or antigen binding fragment comprises a Fc region and wherein the Fc comprises a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2).

In some embodiments of the methods or the systems provided herein, the antibody or antigen binding fragment comprises a Fc region and wherein the Fc comprises a human IgG1 with a substitution selected from 329A, 329G, 329Y, 331S, 236F, 236R, 238A, 238E, 238G, 238H, 238I, 238V, 238 W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 4341, 438G, 439E, 439H, 439Q, 440A, 440D, 440E, 440F, 440M, 440T, and 440V, per Kabat numbering.

In some embodiments of the methods or the systems provided herein, the antibody or antigen binding fragment comprises a Fc region and wherein the Fc comprises a sequence at least about 90%, 91%, 92%, 93%, 94%, 95%, 96%, 97%, 98%, 99%, or 100% identical to any one of SEQ ID NOS: 320-362.

In some embodiments of the methods or the systems provided herein, the antibody or antigen binding fragment comprises a Fc region and wherein the Fc comprises any one of SEQ ID NOs: 401-413 or a sequence at least about 90%, 91%, 92%, 93%, 94%, 95%, 96%, 97%, 98%, or 99% identical to any one of SEQ ID NOs: 401-413.

In some embodiments of the methods or the systems provided herein, the antibody or antigen binding fragment comprises a heavy chain comprising any one of SEQ ID NOs: 501-513 or a sequence at least about 90%, 91%, 92%, 93%, 94%, 95%, 96%, 97%, 98%, or 99% identical to any one of SEQ ID NOs: 501-513.

In some embodiments of the methods or the systems provided herein, the antibody or antigen binding fragment comprises a light chain comprising any one of SEQ ID NO: 514 or a sequence at least about 90%, 91%, 92%, 93%, 94%, 95%, 96%, 97%, 98%, or 99% identical to any one of SEQ 515.

In some embodiments of the methods or the systems provided herein, the inflammatory, fibrotic, or fibrostenotic disease or condition comprises inflammatory bowel disease, Crohn's disease, obstructive Crohn's disease, ulcerative colitis, intestinal fibrosis, intestinal fibrostenosis, rheumatoid arthritis, or primary sclerosing cholangitis. In some embodiments of the methods or the systems provided herein, the Crohn's disease is ileal, ileocolonic, or colonic Crohn's disease.

In some embodiments of the methods or the systems provided herein, the subject has, or is at risk for developing, a non-response or loss-of-response to a standard therapy comprising glucocorticosteroids, anti-TNF therapy, anti-α4β7 therapy, anti-IL 12p40 therapy, or a combination thereof.

In some embodiments of the methods or the systems provided herein, the expression of the TL1Ashort isoform and the expression level of the TL1A long isoform is determined by ELISA, RNA sequencing, PCR, or mass spectrometry.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### 3. INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### 4. BRIEF DESCRIPTION OF THE DRAWINGS

The novel features of the inventive concepts set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a workflow according to an embodiment of the present disclosure for processing a biological sample obtained from a subject to inform the selection of a therapeutic agent to treat a disease or a condition of the subject.

FIG. 2 shows a computer-implemented workflow according to an embodiment of the present disclosure for generating an electronic report to a user, such as a physician, comprising a TNFSF15 profile of a subject based on an analysis of the data on TL1A isoforms and/or proxy markers from the subject.

FIG. 3 shows a computer system that is programmed or otherwise configured to implement methods provided herein.

FIG. 4 shows a computer-implemented workflow according to an embodiment of the present disclosure for producing a TNFSF15 profile.

FIGS. 5-7 show chromatograms for analytical size exclusion chromatography of anti-TL1A antibodies. FIG. 5 shows chromatograms for analytical size exclusion chromatography of antibodies A193, A194, and A195. FIG. 6 shows chromatograms for analytical size exclusion chromatography of antibodies A196, A197, and A198. FIG. 7 shows chromatograms for analytical size exclusion chromatography of antibodies A199, A200, and A201.

FIGS. 8-9 depict inhibition of interferon gamma in human blood with anti-TL1A antibodies. FIG. 8 depicts inhibition of interferon gamma in human blood with anti-TL1A antibodies A219 and A213. FIG. 9 depicts inhibition of interferon gamma in human blood with anti-TL1A antibody A212.

FIGS. 10-12 depicts a PLS model demonstrating effect of pH and protein concentration on viscosity. FIG. 10 shows a PLS graph, FIG. 11 shows a model of the predicted viscosity versus anti-TL1A antibody concentration in mg/mL, and FIG. 12 shows a model of the estimated viscosity versus actual viscosity. Viscosity units are in mPa-s.

FIG. 13 graphically depicts TL1A long isoform and TL1Ashort isoform on chromosome 9. The TL1A long isoform (also known and referred to as the canonical isoform) can comprise the sequence set forth in SEQ ID NO: 2050. The TL1Ashort isoform can comprise the sequence set forth in SEQ ID NO: 2052. The third TL1A mRNA isoform encoding SEQ ID NO: 2051 was not observed by the inventors in gastrointestinal tissues in IBD patients, and not depicted in FIG. 13.

FIG. 14 shows the software design architecture of the assay interpretation software, according to some embodiments herein.

FIGS. 15A-15K show screenshots of the assay interpretation software application from to illustrate the user experience, according to some embodiments herein. The assay interpretation software application comprises a user interface that comprises a login view (FIG. 15A), a files view (FIG. 15B), a samples view (FIG. 15C), a data analyses view (FIG. 15D), a new data analysis view (FIG. 15E), system settings view (FIG. 15F), a clinical settings view (FIG. 15G), a clinical study settings view (FIG. 15H), a user management view (FIG. 151), an activities view (FIG. 15J), or a my profile view (FIG. 15K), or any combination thereof.

FIGS. 16A-16B show exemplary clinical trial studies of the anti-TL1A antibodies disclosed herein. FIG. 16A depicts the study schema for induction period for the phase 2 clinical trial for A219 in UC in accordance with some embodiments herein. FIG. 16B depicts the study schema for open-label extension period for the phase 2 clinical trial for A219 in UC in accordance with some embodiments herein.

FIGS. 17A-17D show mRNA transcript levels of TL1A isoforms in various gastrointestinal tissues. FIG. 17A and FIG. 17B show mRNA transcript levels of TL1A long isoform and TL1Ashort isoform, respectively, in biopsy samples obtained before TL1A inhibitor treatment. FIG. 17C and FIG. 17D show mRNA transcript levels of TL1A long isoform and TL1Ashort isoform, respectively, in biopsy samples obtained both before and after TL1A inhibitor treatment. The horizontal lines in FIGS. 17B and 17D depicts the approximate detection limit if RNAseq is performed at 50 million reads.

FIG. 18 shows both TL1Ashort and long isoforms were detected in ileum across the course of treatment and no statistically significant changes were seen in the ratio between TL1A short isoform and TL1A long isoform across the time points during the treatment. The early termination has very few data points (as very few patients had early termination) and thus is not a statistically significant time point.

FIG. 19 shows in samples of an IBD patient cohort treated and collected at University of California San Diego, both TL1Ashort and long isoforms were detected in ileum and colon tissues and the expression of both TL1Ashort and long isoforms were higher in ileum.

FIGS. 20A-20E show that TL1A isoform ratio has statistically significant association with the CD patients' endoscopic response to the anti-TL1A treatment. Samples were filtered to those with greater than 0 transcripts per million for short isoform and greater than 0.2 transcripts per million for long isoform. In FIG. 20A, a combination of available baseline (before treatment) and after treatment ileal samples were used to determine TL1A isoform ratio. In FIG. 20B, a combination of available baseline-only ileal and colon samples were used to determine TL1A isoform ratio. In FIG. 20C, only baseline ileal samples were used to determine TL1A isoform ratio for association studies with CD patients' endoscopic response to the anti-TL1A treatment. In FIG. 20D, only baseline colon samples were used to determine TL1A isoform ratio for association studies with CD patients' endoscopic response to the anti-TL1A treatment. In FIG. 20E, only baseline rectum samples were used to determine TL1A isoform ratio for association studies with CD patients' endoscopic response to the anti-TL1A treatment.

FIG. 21 shows both TL1Ashort and long isoforms were detected in colon across the course of treatment and no statistically significant changes were seen in the ratio between TL1A short isoform and TL1A long isoform across the time points during the treatment with anti-TL1A (A219) in UC patients.

FIGS. 22A-22B show that TL1A isoform ratio has statistically significant association with the UC patients' positive therapeutic response (e.g. clinical remission and endoscopic improvement) to the anti-TL1A treatment. Samples were filtered based on detectable (at greater than 0 transcript per million) levels of short isoform and detectable (at greater than 0.4 transcripts per million) level of long TL1A isoform. In FIG. 22A, TL1Ashort/long isoform ratio from baseline colon samples of UC patients significantly associated with the clinical remission of the UC patients after anti-TL1A treatment (A219 treatment). In FIG. 22B, UC patients who had endoscopic improvement after anti-TL1A treatment (A219 treatment) had higher TL1A short/long isoform ratio as determined in baseline colon samples of the UC patients.

FIGS. 23A-23C show the risk alleles for rs6478109 SNP correlated with TL1A short/long isoform ratio in the cohort of CD patients with statistical significance (p<0.05). Samples were filtered based on detectable (at greater than 0.1 transcript per million) levels of short and long TL1A isoforms. FIG. 23A shows the transcripts of canonical isoform (long isoform) plotted against the risk alleles of rs6478109, FIG. 23B shows the transcripts of the TL1Ashort isoform plotted against the risk alleles of rs6478109, and FIG. 23C shows the TL1A short/long isoform ratio plotted against the risk alleles of rs6478109. In FIGS. 23A-23C, 0 indicates homozygous for risk allele (minor allele), 1 indicates heterozygous for risk allele (minor allele) and non-risk (major allele), and 2 indicates homozygous for non-risk allele (major allele).

FIGS. 24A-24B show the validation of TL1A isoform ratio as CDx for selecting CD or UC patients that have a positive therapeutic response to treatment with anti-TL1A antibody (A219). FIG. 24A shows the plot of standardized Wilcoxon statistic with TL1A isoform ratio and endoscopic response in CD patients treated with anti-TL1A antibodies (A219). FIG. 24B shows the plot of standardized Wilcoxon statistic with TL1A isoform ratio and clinical remission in UC patients treated with anti-TL1A antibodies (A219). In both FIG. 24A (CD patient clinical result) and 24B (UC patient clinical result), a range of cutoffs for using TL1A isoform ratio as a CDx, a specific cutoff for using TL1A isoform ratio as a CDx, and the performance matrix of CDx with such specific TL1A isoform ratio are indicated.

### 5. DETAILED DESCRIPTION

Provided herein are methods, systems, and kits for treating a subject who may be suitable for treatment with an inhibitor of Tumor Necrosis Factor (Ligand) Superfamily, Member 15 (TL1A) activity or expression, provided the subject is selected based on the TL1Ashort isoform to TL1A long isoform ratio (TL1A isoform ratio or TL1Ashort/long isoform ratio) or a proxy marker thereto. The subject may be a patient, who may be diagnosed with an inflammatory disease, a fibrostenotic disease, or a fibrotic disease, such as inflammatory bowel disease (IBD) or Crohn's disease (CD). The subject may not be a patient, but may be suspected of having the inflammatory disease, the fibrostenotic disease, or the fibrotic disease. The TL1A isoform ratio may, in some cases, be useful for treating the inflammatory fibrostenotic, or fibrotic disease or condition, as mediated by TL1A. The subject, in some embodiments, is treated by administering the inhibitor of TL1A activity or expression (e.g., anti-TL1A antibody) to the subject, provided the TL1A isoform ratio or the proxy marker is detected. In some cases, identifying the subject as being suitable for treatment with the inhibitor of activity or expression is required in order to administer the inhibitor to the subject.

The TL1A isoform ratio described herein are detected using suitable assays and devices (e.g., array, sequencing). In some instances, a sample is obtained from the subject or patient indirectly or directly. In some instances, the sample may be obtained by the subject. In other instances, the sample may be obtained by a healthcare professional, such as a nurse or physician. The sample may be derived from biological fluid or tissue containing gene expression information or genetic information, such as blood or tissue biopsy.

The subject disclosed herein can be a mammal, such as for example a mouse, rat, guinea pig, rabbit, non-human primate, or farm animal. In some instances, the subject is human. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a standard treatment (e.g., anti-TNF alpha therapy, anti-a4-b7 therapy (vedolizumab), anti-IL12p40 therapy (ustekinumab), Thalidomide, or Cytoxin).

The disease or condition disclosed herein may be an inflammatory disease, a fibrostenotic disease, or a fibrotic disease. In some instances, the disease or the condition is a TL1A-mediated disease or condition. The term, “TL1A-mediated disease or condition” refers to a disease or a condition pathology or pathogenesis that is driven, at least in part, by TL1A signaling. In some instances, the disease or the condition is immune-mediated disease or condition, such as those mediated by TL1A.

In some embodiments the disease or the condition is an inflammatory disease or disorder that is mediated, at least in part, by TL1A signaling. Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis, rheumatic disease, scleroderma, sinusitis, tuberculosis.

In some embodiments, the disease or the condition is an autoimmune disease that is mediated, at least in part, by TL1A signaling. Non-limiting examples of autoimmune disease or disorder include Achalasia, Addison's disease, Adult Still's disease, Agammaglobulinemia, Alopecia areata, Amyloidosis. Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease. Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC). Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis. Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or the condition is a cancer that is mediated, at least in part, by TL1A signaling. Non-limiting examples of cancers include Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia-Acute Lymphoblastic Leukemia, Leukemia-Acute Lymphocytic (ALL), Leukemia-Acute Myeloid AML, Leukemia-Adult, Leukemia-Childhood, Leukemia-Chronic Lymphocytic (CLL), Leukemia-Chronic Myeloid (CML), Liver Cancer, Lobular Carcinoma, Lung Cancer, Lung Cancer-Small Cell (SCLC), Lung Cancer-Non-small Cell (NSCLC), Lymphoma-Hodgkin's, Lymphoma-Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma-Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

In some embodiments, the disease or the condition is an inflammatory bowel disease, such as Crohn's disease (CD) or ulcerative colitis (UC). A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotie and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteriods, anti-TNF therapy, anti-a4-b7 therapy (vedolizumab), anti-IL 12p40 therapy (ustekinumab), Thalidomide, and Cytoxin.

Disclosed herein are TL1A isoforms, TL1A isoform ratios, and a proxy maker thereto that may be detected in a sample obtained from a subject by analyzing the nucleic acid and protein material in the sample. In some instances, the subject may be human. In some embodiments, the genetic material is obtained from a subject having a disease or condition disclosed herein. In some cases, the nucleic acid and protein material is obtained from blood, serum, plasma, and/or tissue biopsies by techniques known by one of skill in the art. In some cases, the nucleic acid and protein material is obtained from a biopsy, e.g., from the intestinal track of the subject.

Aspects disclosed herein provide TL1A isoform ratio or a proxy marker thereto that is associated with, and therefore indicative of, a subject associated with an increase TNFSF15 (TL1A) expression or activity contributory to an inflammatory, a fibrotic, or a fibrostenotic disease or condition in the subject (such as CD and UC). Thus, the TL1A isoform ratio or a proxy marker thereto is indicative that the subject will have a positive therapeutic response to an inhibitor of TL1A activity or expression. The term, “positive therapeutic response” refers to a reduction or an elimination of at least one symptom of the disease or the condition (e.g., Cohn's disease) after induction of a therapy (e.g., anti-TL1A antibody).

The TL1A isoform ration or the proxy marker thereof has already been validated to correlate with an increase of a positive therapeutic response in IBD patients to a treatment with the inhibitor of TL1A activity or expression, as further described in Section 7 (EXAMPLES). As such, the disclosure provides that the TL1A isoform ratio or the proxy marker thereof provided herein can be used as criteria for identifying subjects or patients for the various methods provided herein including in Sections 2, 5.1, and 7 (EXAMPLES). Similarly, the disclosure provides that the TL1A isoform ratio or the proxy marker thereof provided herein can be used as criteria for identifying subjects or patients for the various kits and compositions provided herein including in Sections 2, 5.4, 5.5, and 7 (EXAMPLES). Additionally, the disclosure provides that the TL1A isoform ratio or the proxy marker thereof provided herein can be used as criteria for the various methods of selecting patients provided herein including in Sections 2, 5.1, and 7.

The disclosure further provides simple methods to validate the suitability of the TL1A isoform ratio or the proxy marker thereof for the various methods of treatment, methods of selecting patients, and/or the kits and compositions provided herein including in Sections 2, 5.1, 5.4, 5.5, and 7.

The TL1A isoform ratio or the proxy marker thereof provided in the Examples may be used to predict a positive therapeutic response in a subject or a patient to an inhibitor of TL1A activity or expression (e.g., anti-TL1A antibody), either alone, or in combinations. The TL1A isoform ratio or the proxy marker thereof described herein may be used in a diagnostic or prognostic test to identify a subject suitable for treatment with an inhibitor of TL1A activity or expression to treat a disease or condition described herein in the subject. In some cases, the diagnostic is a companion diagnostic test, such as for example, a TL1A companion diagnostic test (“TL1A CDx”).

To validate the CDx based on the TL1A isoform ratio or the proxy marker thereof, an external cohort of IBD (e.g., UC or CD) patients can be identified and their biopsied tissue samples assayed to determine the TL1A isoform ratio or the proxy marker thereof. The positive predictive value, negative predictive value, specificity, sensitivity, and positive rate of the patient population can be calculated from the patients that have the TL1A isoform ratio or the proxy marker and the clinical response or clinical remission of the IBD patients after the TL1A inhibitor therapy, for example as described in Section 7 (Examples). The cutoff value for the TL1A isoform ratio used to make CDx positive or CDx negative determination for a given positive predictive value, negative predictive value, specificity, sensitivity, and/or positive rate can be identified by the machine learning algorithms and validated as provided herein without undue experimentation.

In some embodiments, the elevated TL1A expression and elevated TL1A activity in this Section (including in the preceding paragraphs) are in reference to such in a tissue or subject not affected by IBD (e.g. UC or CD).

### 5.1 Methods of Treatment

Disclosed herein are methods of treating a disease or condition, or a symptom of the disease or condition, in a subject, comprising administrating of therapeutic effective amount of one or more therapeutic agents to the subject. In some embodiments, the one or more therapeutic agents is administered to the subject alone (e.g., standalone therapy). In some embodiments, the one or more therapeutic agents is administered in combination with an additional agent. In some embodiments, the therapeutic agent is a first-line therapy for the disease or condition. In some embodiments, the therapeutic agent is a second-line, third-line, or fourth-line therapy, for the disease or condition. In some embodiments, the therapeutic agent comprises an inhibitor of Tumor necrosis factor-like cytokine 1A (TL1A) activity or expression.

Various embodiments provide for methods of treating inflammatory bowel disease (IBD), comprising administering an anti-TL1A antibody described herein to a subject in need thereof. In some embodiments, the subject comprises the TL1A isoform ratio or the proxy marker. In some embodiments, the IBD is a severe form of IBD.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a ratio between TL1A short isoform and TL1A long isoform (“TL1A isoform ratio”), a proxy marker having a statistically significant correlation with the TL1A isoform ratio, or a combination thereof.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a TL1A isoform ratio, a proxy marker having a statistically significant correlation with the TL1A isoform ratio, or a combination thereof;
   (iii) determining the subject is suitable for treatment with an inhibitor of TL1A activity or expression based on the TL1A isoform ratio or the proxy marker; and
(b) treating the subject by administering a therapeutically effective amount of the inhibitor of TL1A activity or expression to the subject.

In a further aspect, provided herein is a method of determining a TL1A isoform ratio or a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect an expression level of the TL1A short isoform and an expression level of the TL1A long isoform; and
(c) determining a ratio TL1A isoform ratio, a proxy marker having a statistically significant correlation with the TL1A isoform ratio, or a combination thereof, wherein whether the subject is suitable for treatment with an inhibitor of TL1A activity or expression is indicated based on the TL1A isoform ratio or the proxy marker.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect an expression level of the TL1A short isoform and an expression level of the TL1A long isoform;
(c) determining a TL1A isoform ratio, a proxy marker having a statistically significant correlation with the TL1A isoform ratio, or a combination thereof; and
(d) selecting the subject for treatment with the inhibitor of TL1A activity or expression based on the TL1A isoform ratio or the proxy marker.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a TL1A isoform ratio.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a TL1A isoform ratio;
   (iii) determining the subject is suitable for treatment with an inhibitor of TL1A activity or expression based on the TL1A isoform ratio; and
(b) treating the subject by administering a therapeutically effective amount of the inhibitor of TL1A activity or expression to the subject.

In a further aspect, provided herein is a method of determining a TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect an expression level of the TL1A short isoform and an expression level of the TL1A long isoform; and
(c) determining a ratio TL1A isoform ratio, wherein whether the subject is suitable for treatment with an inhibitor of TL1A activity or expression is indicated based on the TL1A isoform ratio.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect an expression level of the TL1A short isoform and an expression level of the TL1A long isoform;
(c) determining a TL1A isoform ratio; and
(d) selecting the subject for treatment with the inhibitor of TL1A activity or expression based on the TL1A isoform ratio.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a proxy marker having a statistically significant correlation with the TL1A isoform ratio.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a proxy marker having a statistically significant correlation with the TL1A isoform ratio;
   (iii) determining the subject is suitable for treatment with an inhibitor of TL1A activity or expression based on the proxy marker; and
(b) treating the subject by administering a therapeutically effective amount of the inhibitor of TL1A activity or expression to the subject.

In a further aspect, provided herein is a method of determining a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect the proxy marker; and
(c) determining the proxy marker, wherein whether the subject is suitable for treatment with an inhibitor of TL1A activity or expression is indicated based on the proxy marker.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a proxy marker having a statistically significant correlation with the TL1A isoform ratio;
(c) determining the proxy marker; and
(d) selecting the subject for treatment with the inhibitor of TL1A activity or expression based on the proxy marker.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a proxy marker having a statistically significant correlation with the TL1A isoform ratio, wherein the correlation is positive correlation.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a proxy marker having a statistically significant correlation with the TL1A isoform ratio, wherein the correlation is positive correlation;
   (iii) determining the subject is suitable for treatment with an inhibitor of TL1A activity or expression based on the proxy marker; and
(b) treating the subject by administering a therapeutically effective amount of the inhibitor of TL1A activity or expression to the subject.

In a further aspect, provided herein is a method of determining a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect the proxy marker; and
(c) determining the proxy marker, wherein whether the subject is suitable for treatment with an inhibitor of TL1A activity or expression is indicated based on the proxy marker,
   wherein the correlation is positive correlation.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a proxy marker having a statistically significant correlation with the TL1A isoform ratio, wherein the correlation is positive correlation;
(c) determining the proxy marker; and
(d) selecting the subject for treatment with the inhibitor of TL1A activity or expression based on the proxy marker.

In various method provided herein, including in the preceding paragraphs of this Section, the method further comprises preparing nucleic acids from the sample.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a TL1A isoform ratio, a proxy marker having a statistically significant correlation with the TL1A isoform ratio, or a combination thereof;
   (iv) determining the subject is suitable for treatment with an inhibitor of TL1A activity or expression based on the TL1A isoform ratio or the proxy marker; and
(b) treating the subject by administering a therapeutically effective amount of the inhibitor of TL1A activity or expression to the subject.

In a further aspect, provided herein is a method of determining a TL1A isoform ratio or a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform; and
(d) determining a ratio TL1A isoform ratio, a proxy marker having a statistically significant correlation with the TL1A isoform ratio, or a combination thereof, wherein whether the subject is suitable for treatment with an inhibitor of TL1A activity or expression is indicated based on the TL1A isoform ratio or the proxy marker.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform;
(d) determining a TL1A isoform ratio, a proxy marker having a statistically significant correlation with the TL1A isoform ratio, or a combination thereof; and
(e) selecting the subject for treatment with the inhibitor of TL1A activity or expression based on the TL1A isoform ratio or the proxy marker.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a TL1A isoform ratio;
   (iv) determining the subject is suitable for treatment with an inhibitor of TL1A activity or expression based on the TL1A isoform ratio; and
(b) treating the subject by administering a therapeutically effective amount of the inhibitor of TL1A activity or expression to the subject.

In a further aspect, provided herein is a method of determining a TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform; and
(d) determining a ratio TL1A isoform ratio, wherein whether the subject is suitable for treatment with an inhibitor of TL1A activity or expression is indicated based on the TL1A isoform ratio.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform;
(d) determining a TL1A isoform ratio; and
(e) selecting the subject for treatment with the inhibitor of TL1A activity or expression based on the TL1A isoform ratio.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a proxy marker having a statistically significant correlation with the TL1A isoform ratio;
   (iv) determining the subject is suitable for treatment with an inhibitor of TL1A activity or expression based on the proxy marker; and
(b) treating the subject by administering a therapeutically effective amount of the inhibitor of TL1A activity or expression to the subject.

In a further aspect, provided herein is a method of determining a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect the proxy marker; and
(d) determining the proxy marker, wherein whether the subject is suitable for treatment with an inhibitor of TL1A activity or expression is indicated based on the proxy marker.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect a proxy marker having a statistically significant correlation with the TL1A isoform ratio;
(d) determining the proxy marker; and
(e) selecting the subject for treatment with the inhibitor of TL1A activity or expression based on the proxy marker.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a proxy marker having a statistically significant correlation with the TL1A isoform ratio, wherein the correlation is positive correlation;
   (iv) determining the subject is suitable for treatment with an inhibitor of TL1A activity or expression based on the proxy marker; and
(b) treating the subject by administering a therapeutically effective amount of the inhibitor of TL1A activity or expression to the subject.

In a further aspect, provided herein is a method of determining a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect the proxy marker; and
(d) determining the proxy marker, wherein whether the subject is suitable for treatment with an inhibitor of TL1A activity or expression is indicated based on the proxy marker, wherein the correlation is positive correlation.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect a proxy marker having a statistically significant correlation with the TL1A isoform ratio, wherein the correlation is positive correlation;
(d) determining the proxy marker; and
(e) selecting the subject for treatment with the inhibitor of TL1A activity or expression based on the proxy marker.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a TL1A isoform ratio above a cutoff, a proxy marker above a cutoff, or a combination thereof, wherein the proxy marker has a statistically significant positive correlation with the TL1A isoform ratio.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a TL1A isoform ratio, a proxy marker having a statistically significant positive correlation with the TL1A isoform ratio, or a combination thereof;
   (iii) determining the subject is suitable for treatment with an inhibitor of TL1A activity or expression based on the TL1A isoform ratio above a cutoff or the proxy marker above a cutoff, and
(b) treating the subject by administering a therapeutically effective amount of the inhibitor of TL1A activity or expression to the subject.

In a further aspect, provided herein is a method of determining a TL1A isoform ratio or a proxy marker having a statistically significant positive correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect an expression level of the TL1A short isoform and an expression level of the TL1A long isoform; and
(c) determining the ratio TL1A isoform ratio, the proxy marker, or a combination thereof, wherein whether the subject is suitable for treatment with an inhibitor of TL1A activity or expression is indicated based on the TL1A isoform ratio above a cutoff or the proxy marker above a cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect an expression level of the TL1A short isoform and an expression level of the TL1A long isoform;
(c) determining a TL1A isoform ratio, a proxy marker having a statistically significant positive correlation with the TL1A isoform ratio, or a combination thereof; and
(d) selecting the subject for treatment with the inhibitor of TL1A activity or expression based on the TL1A isoform ratio above a cutoff or the proxy marker above a cutoff.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a TL1A isoform ratio above a cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a TL1A isoform ratio;
   (iii) determining the subject is suitable for treatment with an inhibitor of TL1A activity or expression based on the TL1A isoform ratio above a cutoff; and
(b) treating the subject by administering a therapeutically effective amount of the inhibitor of TL1A activity or expression to the subject.

In a further aspect, provided herein is a method of determining a TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect an expression level of the TL1A short isoform and an expression level of the TL1A long isoform; and
(c) determining a ratio TL1A isoform ratio, wherein whether the subject is suitable for treatment with an inhibitor of TL1A activity or expression is indicated based on the TL1A isoform ratio above a cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect an expression level of the TL1A short isoform and an expression level of the TL1A long isoform;
(c) determining a TL1A isoform ratio; and
(d) selecting the subject for treatment with the inhibitor of TL1A activity or expression based on the TL1A isoform ratio above a cutoff.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a proxy marker above a cutoff, wherein the proxy marker has a statistically significant positive correlation with the TL1A isoform ratio.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a proxy marker having a statistically significant correlation with the TL1A isoform ratio, wherein the correlation is positive correlation;
   (iii) determining the subject is suitable for treatment with an inhibitor of TL1A activity or expression based on the proxy marker above a cutoff; and
(b) treating the subject by administering a therapeutically effective amount of the inhibitor of TL1A activity or expression to the subject.

In a further aspect, provided herein is a method of determining a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect the proxy marker; and
(c) determining the proxy marker, wherein whether the subject is suitable for treatment with an inhibitor of TL1A activity or expression is indicated based on the proxy marker above a cutoff, wherein the correlation is positive correlation.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a proxy marker having a statistically significant correlation with the TL1A isoform ratio, wherein the correlation is positive correlation;
(c) determining the proxy marker; and
(d) selecting the subject for treatment with the inhibitor of TL1A activity or expression based on the proxy marker above a cutoff.

In various method provided herein, including in the preceding paragraphs of this Section, the method further comprises preparing nucleic acids from the sample.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a TL1A isoform ratio, a proxy marker having a statistically significant positive correlation with the TL1A isoform ratio, or a combination thereof;
   (iv) determining the subject is suitable for treatment with an inhibitor of TL1A activity or expression based on the TL1A isoform ratio above a cutoff, the proxy marker above a cutoff or a combination thereof; and
(b) treating the subject by administering a therapeutically effective amount of the inhibitor of TL1A activity or expression to the subject.

In a further aspect, provided herein is a method of determining a TL1A isoform ratio or a proxy marker having a statistically significant positive correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform; and
(d) determining the ratio TL1A isoform ratio, the proxy marker, or a combination thereof, wherein whether the subject is suitable for treatment with an inhibitor of TL1A activity or expression is indicated based on the TL1A isoform ratio above a cutoff, the proxy marker above a cutoff or a combination thereof.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform;
(d) determining a TL1A isoform ratio, a proxy marker having a statistically significant positive correlation with the TL1A isoform ratio, or a combination thereof; and
(e) selecting the subject for treatment with the inhibitor of TL1A activity or expression based on the TL1A isoform ratio above a cutoff, the proxy marker above a cutoff, or a combination thereof.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a TL1A isoform ratio;
   (iv) determining the subject is suitable for treatment with an inhibitor of TL1A activity or expression based on the TL1A isoform ratio above a cutoff; and
(b) treating the subject by administering a therapeutically effective amount of the inhibitor of TL1A activity or expression to the subject.

In a further aspect, provided herein is a method of determining a TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform; and
(d) determining a ratio TL1A isoform ratio, wherein whether the subject is suitable for treatment with an inhibitor of TL1A activity or expression is indicated based on the TL1A isoform ratio above a cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform;
(d) determining a TL1A isoform ratio; and
(e) selecting the subject for treatment with the inhibitor of TL1A activity or expression based on the TL1A isoform ratio above a cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a proxy marker having a statistically significant positive correlation with the TL1A isoform ratio;
   (iv) determining the subject is suitable for treatment with an inhibitor of TL1A activity or expression based on the proxy marker above a cutoff; and
(b) treating the subject by administering a therapeutically effective amount of the inhibitor of TL1A activity or expression to the subject.

In a further aspect, provided herein is a method of determining a proxy marker having a statistically positive significant correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect the proxy marker; and
(d) determining the proxy marker, wherein whether the subject is suitable for treatment with an inhibitor of TL1A activity or expression is indicated based on the proxy marker above a cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect a proxy marker having a statistically significant positive correlation with the TL1A isoform ratio;
(d) determining the proxy marker; and
(e) selecting the subject for treatment with the inhibitor of TL1A activity or expression based on the proxy marker above a cutoff.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a TL1A isoform ratio above a cutoff, a proxy marker below a cutoff, or a combination thereof, wherein the proxy marker has a statistically significant negative correlation with the TL1A isoform ratio.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a TL1A isoform ratio, a proxy marker having a statistically significant negative correlation with the TL1A isoform ratio, or a combination thereof;
   (iii) determining the subject is suitable for treatment with an inhibitor of TL1A activity or expression based on the TL1A isoform ratio above a cutoff, the proxy marker below a cutoff, or a combination thereof; and
(b) treating the subject by administering a therapeutically effective amount of the inhibitor of TL1A activity or expression to the subject.

In a further aspect, provided herein is a method of determining a TL1A isoform ratio or a proxy marker having a statistically significant negative correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect an expression level of the TL1A short isoform and an expression level of the TL1A long isoform; and
(c) determining the ratio TL1A isoform ratio, the proxy marker, or a combination thereof, wherein whether the subject is suitable for treatment with an inhibitor of TL1A activity or expression is indicated based on the TL1A isoform ratio above a cutoff, the proxy marker below a cutoff, or a combination thereof.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect an expression level of the TL1A short isoform and an expression level of the TL1A long isoform;
(c) determining a TL1A isoform ratio, a proxy marker having a statistically significant negative correlation with the TL1A isoform ratio, or a combination thereof; and
(d) selecting the subject for treatment with the inhibitor of TL1A activity or expression based on the TL1A isoform ratio above a cutoff, the proxy marker below a cutoff, or a combination thereof.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a proxy marker below a cutoff, wherein the proxy marker has a statistically significant negative correlation with the TL1A isoform ratio.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a proxy marker having a statistically significant correlation with the TL1A isoform ratio, wherein the correlation is negative correlation;
   (iii) determining the subject is suitable for treatment with an inhibitor of TL1A activity or expression based on the proxy marker below a cutoff; and
(b) treating the subject by administering a therapeutically effective amount of the inhibitor of TL1A activity or expression to the subject.

In a further aspect, provided herein is a method of determining a proxy marker having a statistically significant negative correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect the proxy marker; and
(c) determining the proxy marker, wherein whether the subject is suitable for treatment with an inhibitor of TL1A activity or expression is indicated based on the proxy marker below a cutoff.
   wherein the correlation is negative correlation.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a proxy marker having a statistically significant correlation with the TL1A isoform ratio, wherein the correlation is negative correlation;
(c) determining the proxy marker; and
(d) selecting the subject for treatment with the inhibitor of TL1A activity or expression based on the proxy marker below a cutoff.

In various method provided herein, including in the preceding paragraphs of this Section, the method further comprises preparing nucleic acids from the sample.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a TL1A isoform ratio, a proxy marker having a statistically significant negative correlation with the TL1A isoform ratio, or a combination thereof;
   (iv) determining the subject is suitable for treatment with an inhibitor of TL1A activity or expression based on the TL1A isoform ratio above a cutoff, the proxy marker below a cutoff or a combination thereof; and
(b) treating the subject by administering a therapeutically effective amount of the inhibitor of TL1A activity or expression to the subject.

In a further aspect, provided herein is a method of determining a TL1A isoform ratio or a proxy marker having a statistically significant negative correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform; and
(d) determining the ratio TL1A isoform ratio, the proxy marker, or a combination thereof, wherein whether the subject is suitable for treatment with an inhibitor of TL1A activity or expression is indicated based on the TL1A isoform ratio above a cutoff, the proxy marker below a cutoff or a combination thereof.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect an expression level of the TL1A short isoform and an expression level of the TL1A long isoform;
(d) determining a TL1A isoform ratio, a proxy marker having a statistically significant negative correlation with the TL1A isoform ratio, or a combination thereof; and
(e) selecting the subject for treatment with the inhibitor of TL1A activity or expression based on the TL1A isoform ratio above a cutoff, the proxy marker below a cutoff, or a combination thereof.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a proxy marker having a statistically significant negative correlation with the TL1A isoform ratio;
   (iv) determining the subject is suitable for treatment with an inhibitor of TL1A activity or expression based on the proxy marker below a cutoff; and
(b) treating the subject by administering a therapeutically effective amount of the inhibitor of TL1A activity or expression to the subject.

In a further aspect, provided herein is a method of determining a proxy marker having a statistically negative significant correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect the proxy marker; and
(d) determining the proxy marker, wherein whether the subject is suitable for treatment with an inhibitor of TL1A activity or expression is indicated based on the proxy marker below a cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect a proxy marker having a statistically significant negative correlation with the TL1A isoform ratio;
(d) determining the proxy marker; and
(e) selecting the subject for treatment with the inhibitor of TL1A activity or expression based on the proxy marker below a cutoff.

In one aspect, provided herein is method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a proxy marker having a statistically significant negative correlation with the TL1A isoform ratio according to (i) or (ii):
(i) the subject is selected based on the proxy marker above a cutoff if the proxy marker and the TL1A isoform ratio has a positive correlation (such proxy marker as “positive proxy marker”); or
(ii) the subject is selected based on the proxy marker below a cutoff if the proxy marker and the TL1A isoform ratio has a negative correlation (such proxy marker as “inverse proxy marker”).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to determine a proxy marker having a statistically significant correlation with the TL1A isoform ratio;
   (iii) determining the subject is suitable for treatment with an inhibitor of TL1A activity or expression based on the proxy marker according to (1) the subject is suitable based on the proxy marker above a cutoff if the proxy marker and the TL1A isoform ratio has a positive correlation; or (2) the subject is suitable based on the proxy marker below a cutoff if the proxy marker and the TL1A isoform ratio has a negative correlation; and
(b) treating the subject by administering a therapeutically effective amount of the inhibitor of TL1A activity or expression to the subject.

In a further aspect, provided herein is a method of determining a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect the proxy marker; and
(c) determining the proxy marker, wherein whether the subject is suitable for treatment with an inhibitor of TL1A activity or expression is indicated based on the proxy marker according to (i) the subject is suitable based on the proxy marker above a cutoff if the proxy marker and the TL1A isoform ratio has a positive correlation; or (ii) the subject is suitable based on the proxy marker below a cutoff if the proxy marker and the TL1A isoform ratio has a negative correlation.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a proxy marker having a statistically significant correlation with the TL1A isoform ratio;
(c) determining the proxy marker; and
(d) selecting the subject for treatment with the inhibitor of TL1A activity or expression based on the proxy marker according to (i) the subject is selected based on the proxy marker above a cutoff if the proxy marker and the TL1A isoform ratio has a positive correlation; or (ii) the subject is selected based on the proxy marker below a cutoff if the proxy marker and the TL1A isoform ratio has a negative correlation.

In various method provided herein, including in the preceding paragraphs of this Section, the method further comprises preparing nucleic acids from the sample.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing nucleic acids from the sample;
   (iii) subjecting the sample to an assay adapted to determine a proxy marker having a statistically significant correlation with the TL1A isoform ratio;
   (iv) determining the subject is suitable for treatment with an inhibitor of TL1A activity or expression based on the proxy marker according to (1) the subject is suitable based on the proxy marker above a cutoff if the proxy marker and the TL1A isoform ratio has a positive correlation; or (2) the subject is suitable based on the proxy marker below a cutoff if the proxy marker and the TL1A isoform ratio has a negative correlation; and
(b) treating the subject by administering a therapeutically effective amount of the inhibitor of TL1A activity or expression to the subject.

In a further aspect, provided herein is a method of determining a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) preparing nucleic acids from the sample;
(c) subjecting the sample to an assay adapted to detect the proxy marker; and
(d) determining the proxy marker, wherein whether the subject is suitable for treatment with an inhibitor of TL1A activity or expression is indicated based on the proxy marker according to (i) the subject is suitable based on the proxy marker above a cutoff if the proxy marker and the TL1A isoform ratio has a positive correlation; or (ii) the subject is suitable based on the proxy marker below a cutoff if the proxy marker and the TL1A isoform ratio has a negative correlation.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing nucleic acids from the sample;
(c) subjecting the sample to an assay adapted to detect a proxy marker having a statistically significant correlation with the TL1A isoform ratio;
(d) determining the proxy marker; and
(e) selecting the subject for treatment with the inhibitor of TL1A activity or expression based on the proxy marker according to (i) the subject is suitable based on the proxy marker above a cutoff if the proxy marker and the TL1A isoform ratio has a positive correlation; or (ii) the subject is suitable based on the proxy marker below a cutoff if the proxy marker and the TL1A isoform ratio has a negative correlation.

As is clear from the descriptions herein, the proxy marker can have a positive correlation with the TL1A isoform ratio. When the proxy marker has a positive correlation with the TL1A isoform, the proxy marker moves in the same direction as the TL1A isoform ratio with regard to increased probability of positive therapeutic response to an anti-TL1A treatment in IBD patients. Specifically, when the proxy marker has a positive correlation with the TL1A isoform ratio, the higher the level of the proxy marker in an IBD patient, the higher the probability that the IBD patient will have a positive therapeutic response to an anti-TL1A treatment. Conversely, when the proxy marker moves in the same direction as the TL1A isoform ratio with regard to increased probability of positive therapeutic response to an anti-TL1A treatment in IBD patients. Accordingly, when the proxy marker has a negative correlation with the TL1A isoform ratio, the higher the level of the proxy marker in an IBD patient, the higher the probability that the IBD patient will have a positive therapeutic response to an anti-TL1A treatment.

In various embodiments of the methods provided herein including in Sections 2, 5.1 (e.g. the preceding paragraphs), and 7, the method further comprises preparing DNA from the sample.

In various embodiments of the methods provided herein including in Sections 2, 5.1 (e.g. the preceding paragraphs), and 7, the correlation is Pearson correlation or Spearman correlation. In some embodiments, the correlation is Pearson correlation. In certain embodiments, the correlation is Spearman correlation.

The term “proxy marker” refers to a marker having statistically significant correlation with TL1A isoform ratio at least in a subrange of the TL1A isoform ratio and thus having information for selecting patients having positive therapeutic response to anti-TL LA treatment, because the TL1A isoform ratio itself provides information for selecting patients having positive therapeutic response to anti-TL1A treatment as demonstrated in Examples. Such proxy maker can have statistically significant correlation with TL1A isoform ratio over just a subrange of such the TL1A isoform ratio, so long as such subrange statistically distinguishes the population with high TL1Aₛₕₒᵣₜ/TL1A_{long} ratio from the population with low TL1Aₛₕₒᵣₜ/TL1A_{long} ratio. In some embodiments, the proxy marker has statistically significant correlation with such TL1Aₛₕₒᵣₜ/TL1A_{long} ratio at least in a subrange that encompasses the mean or median of the TL1Aₛₕₒᵣₜ/TL1A_{long} ratio. In some embodiments, the proxy marker has statistically significant correlation with such TL1Aₛₕₒᵣₜ/TL1A_{long} ratio at least in a subrange that encompasses the 2nd and 3rd quartile of the TL1Aₛₕₒᵣₜ/TL1A_{long} ratio. In some embodiments, the proxy marker has statistically significant correlation such TL1Ashort/TL1A_{long} ratio at least in a subrange that encompasses the 2nd quartile of the TL1Aₛₕₒᵣₜ/TL1A_{long} ratio. In some embodiments, the proxy marker has statistically significant correlation with such TL1Aₛₕₒᵣₜ/TL1A_{long} ratio at least in a subrange that encompasses the 3rd quartile of the TL1Aₛₕₒᵣₜ/TL1A_{long} ratio. Accordingly, such proxy marker can be used in lieu of the TL1A isoform ratio for selecting patients having positive therapeutic response to anti-TL1A treatment. In some examples of the methods provided herein including in Sections 2, 5.1 (e.g. the preceding paragraphs), and 7, the proxy marker is a serological marker. In some examples of the methods provided herein including in Sections 2, 5.1 (e.g. the preceding paragraphs), and 7, the proxy marker is a genetic marker. In some examples of the methods provided herein including in Sections 2, 5.1 (e.g. the preceding paragraphs), and 7, the proxy marker is a histology marker. In some examples of the methods provided herein including in Sections 2, 5.1 (e.g. the preceding paragraphs), and 7, the proxy marker is a protein marker. In some examples of the methods provided herein including in Sections 2, 5.1 (e.g. the preceding paragraphs), and 7, the proxy marker is a gene expression marker. In some examples of the methods provided herein including in Sections 2, 5.1 (e.g. the preceding paragraphs), and 7, the proxy marker is a protein expression marker. In some examples of the methods provided herein including in Sections 2, 5.1 (e.g. the preceding paragraphs), and 7, the proxy marker is a mRNA marker. In some examples of the methods provided herein including in Sections 2, 5.1 (e.g. the preceding paragraphs), and 7, the proxy marker is a single nucleotide polymorphism. In some examples of the methods provided herein including in Sections 2, 5.1 (e.g. the preceding paragraphs), and 7, the proxy marker is rs6478109. In some embodiments, if the proxy marker and the TL1A isoform ratio has a positive correlation (such proxy marker as “positive proxy marker”), the higher the level of the proxy marker the higher the probability the patients can have a positive therapeutic response to anti-TL1A treatment; and if the proxy marker and the TL1A isoform ratio has a negative correlation (such proxy marker as “inverse proxy marker”), the higher the level of the proxy marker the lower the probability the patients can have a positive therapeutic response to anti-TL1A treatment.

Accordingly, the TL1A isoform ratio and/or the proxy marker can be used to indicate the probability of the patient having a therapeutic response to the treatment of an TL1A inhibitor, with higher TL1A isoform ratio indicating higher probability of the patient having a therapeutic response and lower TL1A isoform ratio indicating lower probability of the patient having a therapeutic response. In some examples of the methods provided herein, the TL1A isoform ratio can be used as the basis for binarily classifying a patient as a responder (CDx positive) or non-responder (CDx negative). The cutoff of the TL1A isoform ratio for determining such binary classification of the patient can be determined by training the model with the therapeutic response data from a patient cohort such that the TL1A isoform ratio cutoff provide the highest accuracy for classifying the responder, the non-responder, or both the responder and the non-responder, as described elsewhere in this disclosure. Such TL1A isoform ratio cutoff can also be determined by other machine learning or computerized clustering methods known and practiced in the art and as described elsewhere in this disclosure. In other examples, the mathematical function (e.g. coefficients in the function) used to calculate the TL1A isoform ratio from patient's samples can be trained and normalized such that the TL1A isoform ratio has a range of 0 to 1 and 0.5 is cutoff for optimally classifying patient as a responder (CDx positive) or non-responder (CDx negative) to TL1A inhibitor treatment. In one specific example, the TL1A isoform ratio is calculated as TL1Aₛₕₒᵣₜ/TL1A_{long}, wherein TL1Aₛₕₒᵣₜ is an expression level of the TL1Ashort isoform, TL1A_{long} is an expression level of the TL1A long isoform. In another specific example, the TL1A isoform ratio is calculated as ln(TL1Aₛₕₒᵣₜ/TL1A_{long}). In yet another specific example, the TL1A isoform ratio is calculated as ln(TL1Aₛₕₒᵣₜ/TL1 A_{long}+n), wherein n is a positive number. In general, if the TL1A isoform ratio is ≥ cutoff, the prediction is “yes, responder” and CDx positive and if the TL1A isoform ratio is <cutoff, the prediction is “no, non-responder” and CDx negative.

Similarly and as is already clear from the description herein, the proxy marker can be used to indicate the probability of the patient having a therapeutic response to the treatment of an TL1A inhibitor. If the proxy marker has a positive correlation with TL1A isoform ratio, the higher proxy marker level indicates higher probability of the patient having a therapeutic response and lower proxy marker level indicates lower probability of the patient having a therapeutic response. If the proxy marker has a negative correlation with TL1A isoform ratio, the higher proxy marker level indicates lower probability of the patient having a therapeutic response and lower proxy marker level indicates higher probability of the patient having a therapeutic response. In some examples of the methods provided herein, the proxy marker can be used as the basis for binarily classifying a patient as a responder (CDx positive) or non-responder (CDx negative). The cutoff of the proxy marker for determining such binary classification of the patient can be determined by training the model with the therapeutic response data from a patient cohort such that the proxy marker cutoff provides the highest accuracy for classifying the responder, the non-responder, or both the responder and the non-responder, as described elsewhere in this disclosure. Such proxy marker cutoff can also be determined by other machine learning or computerized clustering methods known and practiced in the art and as described elsewhere in this disclosure. In other examples, the mathematical function (e.g. coefficients in the function) used to calculate the proxy marker from patient's samples can be trained and normalized such that the proxy marker has a range of 0 to 1 and 0.5 is cutoff for optimally classifying patient as a responder (CDx positive) or non-responder (CDx negative) to TL1A inhibitor treatment. In general, when the proxy marker has a positive correlation with TL1A isoform ratio, then: if the proxy marker is ≥cutoff, the prediction is “yes, responder” and CDx positive and if the proxy marker is <cutoff, the prediction is “no, non-responder” and CDx negative. When the proxy marker has a negative correlation with TL1A isoform ratio, then: if the proxy marker is >cutoff, the prediction is “no, non-responder” and CDx negative and if the proxy marker is ≤cutoff, the prediction is “yes, responder” and CDx positive.

In some embodiments of the various methods provided herein, including in Sections 2, 5.1, and 7, the correlation between the proxy marker and the TL1A isoform ratio has a correlation coefficient with an absolute value of at least about 0.30, at least about 0.31, at least about 0.32, at least about 0.33, at least about 0.34, at least about 0.35, at least about 0.36, at least about 0.37, at least about 0.38, at least about 0.39, at least about 0.40, at least about 0.41, at least about 0.42, at least about 0.43, at least about 0.44, at least about 0.45, at least about 0.46, at least about 0.47, at least about 0.48, at least about 0.49, at least about 0.50, at least about 0.51, at least about 0.52, at least about 0.53, at least about 0.54, at least about 0.55, at least about 0.56, at least about 0.57, at least about 0.58, at least about 0.59, at least about 0.60, at least about 0.61, at least about 0.62, at least about 0.63, at least about 0.64, at least about 0.65, at least about 0.66, at least about 0.67, at least about 0.68, at least about 0.69, at least about 0.70, at least about 0.71, at least about 0.72, at least about 0.73, at least about 0.74, at least about 0.75, at least about 0.76, at least about 0.77, at least about 0.78, at least about 0.79, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or 1. In other embodiments of the various methods provided herein, including in Sections 2, 5.1, and 7, the correlation between the proxy marker and the TL1A isoform ratio has a correlation coefficient with an absolute value of about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, about 0.99, or 1.

The statistical significance of the correlation can be evaluated by p value calculated from statistical tests. Accordingly, in some embodiments of the various methods provided herein, including in Sections 2, 5.1, and 7, the statistical significance of the correlation between the proxy marker and the TL1A isoform ratio is determined by p value no more than 0.1 in a statistical test. In some embodiments, the statistical significance of the correlation between the proxy marker and the TL1A isoform ratio is determined by p value no more than 0.09 in a statistical test. In some embodiments, the statistical significance of the correlation between the proxy marker and the TL1A isoform ratio is determined by p value no more than 0.08 in a statistical test. In some embodiments, the statistical significance of the correlation between the proxy marker and the TL1A isoform ratio is determined by p value no more than 0.07 in a statistical test. In some embodiments, the statistical significance of the correlation between the proxy marker and the TL1A isoform ratio is determined by p value no more than 0.06 in a statistical test. In some embodiments, the statistical significance of the correlation between the proxy marker and the TL1A isoform ratio is determined by p value no more than 0.05 in a statistical test. In some embodiments, the statistical significance of the correlation between the proxy marker and the TL1A isoform ratio is determined by p value no more than 0.04 in a statistical test. In some embodiments, the statistical significance of the correlation between the proxy marker and the TL1A isoform ratio is determined by p value no more than 0.03 in a statistical test. In some embodiments, the statistical significance of the correlation between the proxy marker and the TL1A isoform ratio is determined by p value no more than 0.02 in a statistical test. In some embodiments, the statistical significance of the correlation between the proxy marker and the TL1A isoform ratio is determined by p value no more than 0.01 in a statistical test. In some embodiments of the various methods provided herein, including in Sections 2, 5.1, and 7 and in this paragraph, the statistical test is an ANOVA test. In some embodiments of the various methods provided herein, including in Sections 2, 5.1, and 7 and in this paragraph, the statistical test is a linear regression test. In some embodiments of the various methods provided herein, including in Sections 2, 5.1, and 7 and in this paragraph, the statistical test is a logistic regression test. In some embodiments of the various methods provided herein, including in Sections 2, 5.1, and 7 and in this paragraph, the statistical test is a T-test. In some embodiments of the various methods provided herein, including in Sections 2, 5.1, and 7 and in this paragraph, the statistical test is Pearson correlation test. In some embodiments of the various methods provided herein, including in Sections 2, 5.1, and 7 and in this paragraph, the statistical test is a Chi-square test.

In some embodiments of the various methods provided herein, including in Sections 2, 5.1, and 7, the proxy marker positively correlates with TL1A isoform ratio with a correlation coefficient of at least about 0.6. In some embodiments of the various methods provided herein, the proxy marker positively correlates with TL1A isoform ratio with a correlation coefficient of at least about 0.65. In some embodiments of the various methods provided herein, the proxy marker positively correlates with TL1A isoform ratio with a correlation coefficient of at least about 0.7. In some embodiments of the various methods provided herein, the proxy marker positively correlates with TL1A isoform ratio with a correlation coefficient of at least about 0.75. In some embodiments of the various methods provided herein, the proxy marker positively correlates with TL1A isoform ratio with a correlation coefficient of at least about 0.8. In some embodiments of the various methods provided herein, the proxy marker positively correlates with TL1A isoform ratio with a correlation coefficient of at least about 0.85. In some embodiments of the various methods provided herein, the proxy marker positively correlates with TL1A isoform ratio with a correlation coefficient of at least about 0.9. In some embodiments of the various methods provided herein, the proxy marker positively correlates with TL1A isoform ratio with a correlation coefficient of at least about 0.95. In some embodiments of the various methods provided herein, the proxy marker positively correlates with TL1A isoform ratio with a correlation coefficient of at least about 0.99.

In some embodiments of the various methods provided herein, including in Sections 2, 5.1, and 7, the proxy marker negatively correlates with TL1A isoform ratio with a correlation coefficient of at most about −0.6. In some embodiments of the various methods provided herein, the proxy marker negatively correlates with TL1A isoform ratio with a correlation coefficient of at most about −0.65. In some embodiments of the various methods provided herein, the proxy marker negatively correlates with TL1A isoform ratio with a correlation coefficient of at most about −0.7. In some embodiments of the various methods provided herein, the proxy marker negatively correlates with TL1A isoform ratio with a correlation coefficient of at most about −0.75. In some embodiments of the various methods provided herein, the proxy marker negatively correlates with TL1A isoform ratio with a correlation coefficient of at most about −0.8. In some embodiments of the various methods provided herein, the proxy marker negatively correlates with TL1A isoform ratio with a correlation coefficient of at most about −0.85. In some embodiments of the various methods provided herein, the proxy marker negatively correlates with TL1A isoform ratio with a correlation coefficient of at most about −0.9. In some embodiments of the various methods provided herein, the proxy marker negatively correlates with TL1A isoform ratio with a correlation coefficient of at most about −0.95. In some embodiments of the various methods provided herein, the proxy marker negatively correlates with TL1A isoform ratio with a correlation coefficient of at most about −0.99.

In some embodiments of the various methods provided herein, including in Sections 2, 5.1, and 7, the proxy marker positively correlates with TL1A isoform ratio with a correlation coefficient of about 0.6. In some embodiments of the various methods provided herein, the proxy marker positively correlates with TL1A isoform ratio with a correlation coefficient of about 0.65. In some embodiments of the various methods provided herein, the proxy marker positively correlates with TL1A isoform ratio with a correlation coefficient of about 0.7. In some embodiments of the various methods provided herein, the proxy marker positively correlates with TL1A isoform ratio with a correlation coefficient of about 0.75. In some embodiments of the various methods provided herein, the proxy marker positively correlates with TL1A isoform ratio with a correlation coefficient of about 0.8. In some embodiments of the various methods provided herein, the proxy marker positively correlates with TL1A isoform ratio with a correlation coefficient of about 0.85. In some embodiments of the various methods provided herein, the proxy marker positively correlates with TL1A isoform ratio with a correlation coefficient of about 0.9. In some embodiments of the various methods provided herein, the proxy marker positively correlates with TL1A isoform ratio with a correlation coefficient of about 0.95. In some embodiments of the various methods provided herein, the proxy marker positively correlates with TL1A isoform ratio with a correlation coefficient of about 0.99.

In some embodiments of the various methods provided herein, including in Sections 2, 5.1, and 7, the proxy marker negatively correlates with TL1A isoform ratio with a correlation coefficient of about −0.6. In some embodiments of the various methods provided herein, the proxy marker negatively correlates with TL1A isoform ratio with a correlation coefficient of about −0.65. In some embodiments of the various methods provided herein, the proxy marker negatively correlates with TL1A isoform ratio with a correlation coefficient of about −0.7. In some embodiments of the various methods provided herein, the proxy marker negatively correlates with TL1A isoform ratio with a correlation coefficient of about −0.75. In some embodiments of the various methods provided herein, the proxy marker negatively correlates with TL1A isoform ratio with a correlation coefficient of about −0.8. In some embodiments of the various methods provided herein, the proxy marker negatively correlates with TL1A isoform ratio with a correlation coefficient of about −0.85. In some embodiments of the various methods provided herein, the proxy marker negatively correlates with TL1A isoform ratio with a correlation coefficient of about −0.9. In some embodiments of the various methods provided herein, the proxy marker negatively correlates with TL1A isoform ratio with a correlation coefficient of about −0.95. In some embodiments of the various methods provided herein, the proxy marker negatively correlates with TL1A isoform ratio with a correlation coefficient of about −0.99.

In one aspect, provided herein is a method of identifying a proxy marker for a ratio between TL1Ashort isoform and TL1A long isoform (“TL LA isoform ratio”), the method comprising:
(a) determine the TL1A isoform ratio in a population of subjects;
(b) determining a marker other than the TL1A isoform ratio in the population of subjects,
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio.

In one aspect, provided herein is a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio,
   wherein the proxy marker relative to a cutoff is indicative that the subject is suitable for treatment with an inhibitor of TL1A activity or expression for treatment of the inflammatory, fibrotic, or fibrostenotic disease or condition.

In one aspect, provided herein is a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio, wherein the proxy marker and the TL1A isoform ratio has a positive correlation.

In one aspect, provided herein is a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio,
   wherein the proxy marker relative to a cutoff is indicative that the subject is suitable for treatment with an inhibitor of TL1A activity or expression for treatment of the inflammatory, fibrotic, or fibrostenotic disease or condition and wherein the proxy marker and the TL1A isoform ratio has a positive correlation.

In some embodiments of the various methods provided herein, including in Sections 2, 5.1, and 7, the proxy marker is identified by a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio.

In some embodiments of the various methods provided herein, including in Sections 2, 5.1, and 7, the proxy marker is identified by a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio wherein the proxy marker and the TL1A isoform ratio has a positive correlation.

In some embodiments of the various methods provided herein, including in Sections 2, 5.1, and 7, the proxy marker is identified by a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio.
   wherein the proxy marker relative to a cutoff is indicative that the subject is suitable for treatment with an inhibitor of TL1A activity or expression for treatment of the inflammatory, fibrotic, or fibrostenotic disease or condition.

In some embodiments of the various methods provided herein, including in Sections 2, 5.1, and 7, the proxy marker is identified by a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio,
   wherein the proxy marker relative to a cutoff is indicative that the subject is suitable for treatment with an inhibitor of TL1A activity or expression for treatment of the inflammatory, fibrotic, or fibrostenotic disease or condition and wherein the proxy marker and the TL1A isoform ratio has a positive correlation.

In one aspect, provided herein is a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio, wherein the proxy marker and the TL1A isoform ratio has a negative correlation.

In one aspect, provided herein is a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects.
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio,
   wherein the proxy marker relative to a cutoff is indicative that the subject is suitable for treatment with an inhibitor of TL1A activity or expression for treatment of the inflammatory, fibrotic, or fibrostenotic disease or condition and wherein the proxy marker and the TL1A isoform ratio has a negative correlation.

In some embodiments of the various methods provided herein, including in Sections 2, 5.1, and 7, the proxy marker is identified by a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio
   wherein the proxy marker and the TL1A isoform ratio has a negative correlation.

In some embodiments of the various methods provided herein, including in Sections 2, 5.1, and 7, the proxy marker is identified by a method of identifying a proxy marker for a ratio between TL1Ashort isoform and TL1A long isoform (“TL1A isoform ratio”), the method comprising:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio,
   wherein the proxy marker relative to a cutoff is indicative that the subject is suitable for treatment with an inhibitor of TL1A activity or expression for treatment of the inflammatory, fibrotic, or fibrostenotic disease or condition and wherein the proxy marker and the TL1A isoform ratio has a negative correlation.

In some embodiments of the various methods provided herein, including in Sections 2, 5.1, and 7, the element “based on the TL1A isoform ratio” in the method is based on TL1A isoform ratio above a cutoff.

In some embodiments of the various methods provided herein, including in Sections 2, 5.1, and 7, the element “TL1A isoform ratio relative to the cutoff”′ in the method is the TL1A isoform ratio above the cutoff.

In some embodiments of the various methods provided herein, including in Sections 2, 5.1, and 7, the proxy marker comprises rs6478109. In some embodiments of the various methods provided herein, including in Sections 2, 5.1, and 7, the proxy marker comprises rs6478109 with one or two risk alleles. In some embodiments of the various methods provided herein, including in Sections 2, 5.1, and 7, the proxy marker comprises rs6478109 with one or two minor alleles. In some embodiments of the various methods provided herein, including in Sections 2, 5.1, and 7, the proxy marker comprises rs6478109 with one or two risk alleles, wherein the risk allele comprises an A at the nucleoposition of polymorphism. In some embodiments of the various methods provided herein, including in Sections 2, 5.1, and 7, the proxy marker comprises rs6478109 with one or two minor alleles, wherein the minor allele comprises an A at the nucleoposition of polymorphism.

As is already clear from the description herein, when the proxy marker comprises a SNP, the SNP can be converted into numerical number and a CDx positive or CDx negative determination can be made according to the proxy marker above a cutoff. For example, the polymorphisms can be mathematically represented as: (i) 0 for homozygous nonrisk alleles, 1 for heterozygous nonrisk and risk alleles, and 2 for homozygous risk alleles; (ii) 1 for homozygous nonrisk alleles, 1 for heterozygous nonrisk and risk alleles, and 0 for homozygous risk alleles; (iii) 0 for homozygous nonrisk alleles, 1 for heterozygous nonrisk and risk alleles, and 1 for homozygous risk alleles; (iv) 0 for homozygous nonrisk alleles, 0 for heterozygous nonrisk and risk alleles, and 1 for homozygous risk alleles; (v) 1 for homozygous nonrisk alleles, 0 for heterozygous nonrisk and risk alleles, and 0 for homozygous risk alleles; (vi) 2 for homozygous nonrisk alleles, 1 for heterozygous nonrisk and risk alleles, and 0 for homozygous risk alleles; and/or (vii) 0 for homozygous nonrisk alleles, 1 for heterozygous nonrisk and risk alleles, and 0 for homozygous risk alleles. When the proxy marker has positive correlation with the TL1A isoform ratio, e.g. the proxy marker comprising rs6478109 that is encoded as (1) or (iii) in the 2nd clause of this paragraph, proxy marker above 0 (rs6478109=1 or 2) could be used to select patients having a positive therapeutic response to anti-TL1A treatment (in which case the cutoff=0). When the proxy marker has positive correlation with the TL1A isoform ratio, e.g. the proxy marker comprising rs6478109 that is encoded as (vi) in the 2nd clause of this paragraph, proxy marker below 2 (rs6478109=1 or 0) could be used to select patients having a positive therapeutic response to anti-TL1A treatment (in which case the cutoff=2). Additionally, when the proxy marker has positive correlation with the TL1A isoform ratio, e.g. the proxy marker comprising rs6478109 that is encoded as (v) in the 2nd clause of this paragraph, proxy marker below 1 (rs6478109=0) could be used to select patients having a positive therapeutic response to anti-TL1A treatment (in which case the cutoff=1).

In various embodiments of the methods provided herein including in Sections 2, 5.1 (e.g. the preceding paragraphs), and 7, the methods comprise interpreting the TL1A isoform ratio, the proxy marker, or the combination thereof. In some embodiments, the interpreting step is performed by an interpretation software. In certain embodiments, the interpretation software is as described in Sections 5.6.1, 5.6.2, 5.6.3, 5.6.4, and 5.6.5. In various embodiments of the methods provided herein including in Sections 2, 5.1 (e.g. the preceding paragraphs), and 7, the methods comprise interpreting the TL1A isoform ratio, the proxy marker, or the combination thereof with an interpretation software. In certain embodiments, the step of interpreting the TL1A isoform ratio, the proxy marker, or the combination thereof is any embodiment of such interpreting step as described in Sections 5.6.1, 5.6.2, 5.6.3, 5.6.4, and 5.6.5. In some embodiments, the step of interpreting the TL1A isoform ratio, the proxy marker, or the combination thereof is performed with the software as described in Sections 5.6.1, 5.6.2, 5.6.3, 5.6.4, and 5.6.5. In some embodiments, the step of interpreting the TL1A isoform ratio, the proxy marker, or the combination thereof is performed in a system as described in Sections 5.6.1, 5.6.2, 5.6.3, 5.6.4, and 5.6.5. In some embodiments, the step of interpreting the TL1A isoform ratio, the proxy marker, or the combination thereof is performed with a software as described in Sections 5.6.1, 5.6.2, 5.6.3, 5.6.4, and 5.6.5 and in a system as described in Sections 5.6.1, 5.6.2, 5.6.3, 5.6.4, and 5.6.5.

In various embodiments of the methods provided herein including in Sections 2, 5.1 (e.g. the preceding paragraphs), and 7, the method further comprises preparing nucleic acid from the sample obtained from the subject. In various embodiments of the methods provided herein including in Sections 2, 5.1 (e.g. the preceding paragraphs), and 7, the method further comprises preparing RNA from the sample obtained from the subject. In various embodiments of the methods provided herein including in Sections 2, 5.1 (e.g. the preceding paragraphs), and 7, the method further comprises preparing proteins from the sample obtained from the subject. In various embodiments of the methods provided herein including in Sections 2, 5.1 (e.g. the preceding paragraphs), and 7, the method further comprises isolating nucleic acid from the sample obtained from the subject. In various embodiments of the methods provided herein including in Sections 2, 5.1 (e.g. the preceding paragraphs), and 7, the method further comprises isolating RNA from the sample obtained from the subject. In various embodiments of the methods provided herein including in Sections 2, 5.1 (e.g. the preceding paragraphs), and 7, the method further comprises isolating proteins from the sample obtained from the subject.

In various embodiments of the methods provided herein including in Sections 2, 5.1 (e.g. the preceding paragraphs), and 7, the method further comprises contacting the sample with one or more probes adapted to detect the presence of TL1A long isoform RNA, TL1Ashort isoform RNA, TL1A long isoform protein, and, TL1Ashort isoform protein. In various embodiments of the methods provided herein including in Sections 2, 5.1 (e.g. the preceding paragraphs), and 7, the method further comprises contacting the sample with an assay adapted to detect TL1A long isoform RNA and TL1Ashort isoform RNA. In some embodiments, the method further comprises contacting the sample with an assay adapted to detect TL1A long isoform protein and TL1Ashort isoform protein. In some embodiments, the method further comprises contacting the sample with an assay adapted to detect TL1A long isoform RNA and TL1Ashort isoform protein. In some embodiments, the method further comprises contacting the sample with an assay adapted to detect TL1A long isoform protein and TL1Ashort isoform RNA.

In various embodiments of the methods provided herein including in Sections 2, 5.1 (e.g. the preceding paragraphs), and 7, the method further comprises processing the sample to enrich the RNA. In various embodiments of the methods provided herein including in Sections 2, 5.1 (e.g. the preceding paragraphs), and 7, the method further comprises processing the sample to enrich the protein. In various embodiments of the methods provided herein including in Sections 2, 5.1 (e.g. the preceding paragraphs), and 7, the method further comprises processing the sample to enrich the TL1A RNA. In various embodiments of the methods provided herein including in Sections 2, 5.1 (e.g. the preceding paragraphs), and 7, the method further comprises processing the sample to enrich the TL1A protein.

As is already clear from the descriptions above and below, inverse proxy marker has a negative correlation with the TL1A isoform ratio, therefore patients having a positive therapeutic response to anti-TL1A treatment have the lower level of inverse proxy marker and thus are selected when the inverse proxy marker is below a cutoff. Accordingly, as a result of the negative correlation between the inverse proxy marker and the TL1A isoform ratio, the higher TL1A isoform ratio and the prediction of such higher TL1A isoform ratio can be approximated by lower level of inverse proxy marker. As such, in various embodiments of the methods provided herein including in Sections 2, 5.1 (e.g. the preceding paragraphs), and 7, TL1A isoform ratio or a positive proxy marker above a cutoff is determined if an inverse-proxy marker is below an alternative cutoff, wherein the inverse proxy marker has a negative correlation with the TL1A isoform ratio. In some embodiments of the methods provided herein including in Sections 2, 5.1 (e.g. the preceding paragraphs), and 7, the inverse proxy marker comprises the ratio of TL1A long isoform to the TL1Ashort isoform (the reciprocal of TL1A isoform ratio). In some embodiments of the methods provided herein including in Sections 2, 5.1 (e.g. the preceding paragraphs), and 7, the TL1A isoform ratio above a cutoff is determined if the ratio of TL1A long isoform to the TL1Ashort isoform (the reciprocal of TL1A isoform ratio) is below an alternative cutoff. In certain embodiments, the alternative cutoff for the inverse proxy marker can be determined in correspondence to the cutoff for the TL1A isoform ratio based on the correlation between the inverse proxy marker and TL1A isoform ratio.

In some embodiments of the methods provided herein, including in Sections 2, 5.1, and 7, the cutoff ranges from about 0.090 to about 0.110. In some embodiments of the methods provided herein, including in Sections 2, 5.1, and 7, the cutoff is about 0.097. In some embodiment of the methods provided herein, including in this paragraph, the cutoff is for selecting CD patients. Specifically, in some embodiments of the methods provided herein, including in Sections 2, 5.1, and 7, the cutoff for TL1A isoform ratio ranges from about 0.090 to about 0.110 for selecting CD patients that can have a positive therapeutic response to treatment with an inhibitor of TL1A expression or activity. In some embodiments of the methods provided herein, including in Sections 2, 5.1, and 7, the cutoff for TL1A isoform ratio is about 0.097 for selecting CD patients that can have a positive therapeutic response to treatment with an inhibitor of TL1A expression or activity.

In some embodiments of the methods provided herein, including in Sections 2, 5.1, and 7, the cutoff ranges from about 0.080 to about 0.10. In some embodiments of the methods provided herein, including in Sections 2, 5.1, and 7, the cutoff is about 0.085. In some embodiment of the methods provided herein, including in this paragraph, the cutoff is for selecting UC patients. Specifically, in some embodiments of the methods provided herein, including in Sections 2, 5.1, and 7, the cutoff for TL1A isoform ratio ranges from about 0.080 to about 0.10 for selecting UC patients that can have a positive therapeutic response to treatment with an inhibitor of TL1A expression or activity. In some embodiments of the methods provided herein, including in Sections 2, 5.1, and 7, the cutoff for TL1A isoform ratio is about 0.085 for selecting UC patients that can have a positive therapeutic response to treatment with an inhibitor of TL1A expression or activity.

In some embodiments of the various methods provided herein, including in Sections 2, 5.1, and 7, the cutoff is (i) the TL1A isoform ratio or proxy marker level in a heathy subject without the inflammatory, fibrotic, or fibrostenotic disease or condition or (ii) the TL1A isoform ratio or proxy marker level in a tissue not afflicted by the inflammatory, fibrotic, or fibrostenotic disease or condition. In some embodiments of the various methods provided herein, including in Sections 2, 5.1, and 7, the cutoff is the TL1A isoform ratio or proxy marker level in a heathy subject without the inflammatory, fibrotic, or fibrostenotic disease or condition. In some embodiments of the various methods provided herein, including in Sections 2, 5.1, and 7, the cutoff is the TL1A isoform ratio or proxy marker level in a tissue not afflicted by the inflammatory, fibrotic, or fibrostenotic disease or condition.

The TL1A isoform ratio can be calculated directly as the ratio of the level of TL1A short isoform over the level of TL1A long isoform or some mathematical operation of such ratio. In some embodiments of the methods provided herein, including in Sections 2, 5.1, and 7, the TL1A isoform ratio is calculated as, TL1Aₛₕₒᵣₜ/TL1A_{long}, In (TL1Aₛₕₒᵣₜ/TL1A_{long}), or In (TL1Aₛₕₒᵣₜ/TL1A_{long}+n), wherein TL1Aₛₕₒᵣₜ is an expression level of the TL1Aₛₕₒᵣₜ isoform. TL1A_{long} is an expression level of the TL1A long isoform, and n is a positive number. In some embodiments of the methods provided herein, including in Sections 2, 5.1, and 7, the TL1A isoform ratio is calculated as TL1Aₛₕₒᵣₜ/TL1A_{long}, wherein TL1Aₛₕₒᵣₜ is an expression level of the TL1Aₛₕₒᵣₜ isoform, TL1A_{long} is an expression level of the TL1A long isoform. In some embodiments of the methods provided herein, including in Sections 2, 5.1, and 7, the TL1A isoform ratio is calculated as ln(TL1Aₛₕₒᵣₜ/TL1A_{long}), wherein TL1Aₛₕₒᵣₜ is an expression level of the TL1Aₛₕₒᵣₜ isoform, TL1A_{long} is an expression level of the TL1A long isoform. In some embodiments of the methods provided herein, including in Sections 2, 5.1, and 7, the TL1A isoform ratio is calculated as ln(TL1Aₛₕₒᵣₜ/TL1A_{long}+n), wherein TL1Aₛₕₒᵣₜ is an expression level of the TL1Aₛₕₒᵣₜ isoform, TL1A_{long} is an expression level of the TL1A Jong isoform and “n” is a positive number. In some embodiments of the methods provided herein, including in Sections 2, 5.1, and 7, nis 1.

The level of TL1A long or TL1Aₛₕₒᵣₜ isoforms used in the determination of TL1A isoform ratio can be TL1A mRNA level (gene expression), TL1A protein level (protein expression), or a combination of the two. Accordingly, in one embodiment of the methods provided herein, including in Sections 2, 5.1, and 7, the expression level of the TL1Aₛₕₒᵣₜ isoform is the level of gene expression of the TL1Aₛₕₒᵣₜ isoform and the expression level of the TL1A long isoform is the level of gene expression of the TL1A long isoform. In another embodiment of the methods provided herein, including in Sections 2, 5.1, and 7, the expression level of the TL1Aₛₕₒᵣₜ isoform is the level of protein expression of the TL1Aₛₕₒᵣₜ isoform and the expression level of the TL1A long isoform is the level of protein expression of the TL1A long isoform. In a further embodiment of the methods provided herein, including in Sections 2, 5.1, and 7, the expression level of the TL1Aₛₕₒᵣₜ isoform is the level of gene expression of the TL1Aₛₕₒᵣₜ isoform and the expression level of the TL1A long isoform is the level of protein expression of the TL1A long isoform. In yet another embodiment of the methods provided herein, including in Sections 2, 5.1, and 7, the expression level of the TL1Aₛₕₒᵣₜ isoform is the level of protein expression of the TL1Aₛₕₒᵣₜ isoform and the expression level of the TL1A long isoform is the level of gene expression of the TL1A long isoform. Furthermore, in one embodiment of the methods provided herein, including in Sections 2, 5.1, and 7, the expression level of the TL1Aₛₕₒᵣₜ isoform is the gene expression level. In one embodiment of the methods provided herein, including in Sections 2, 5.1, and 7, the expression level of the TL1Aₛₕₒᵣₜ isoform is the protein expression level. In one embodiment of the methods provided herein, including in Sections 2, 5.1, and 7, the expression level of the TL1A long isoform is the gene expression level. In one embodiment of the methods provided herein, including in Sections 2, 5.1, and 7, the expression level of the TL1A long isoform is the protein expression level. Gene expression in

In various embodiments of the methods provided herein including in Sections 2, 5.1 (e.g. the preceding paragraphs), and 7, the TL1A isoform ratio or the proxy marker is predictive of a positive therapeutic response in the subject to a treatment with the inhibitor of TL1A activity or expression with a positive predictive value of at least 25%, The other embodiments described in this paragraph are provided for the various methods described herein including in Sections 2, 5.1 (e.g. the preceding paragraphs), and 7. In some embodiments, the TL1A isoform ratio or the proxy marker is predictive of a positive therapeutic response in the subject to a treatment with the inhibitor of TL1A activity or expression with a positive predictive value of at least 26%, In some embodiments, the TL1A isoform ratio or the proxy marker is predictive of a positive therapeutic response in the subject to a treatment with the inhibitor of TL1A activity or expression with a positive predictive value of at least 27%, In some embodiments, the TL1A isoform ratio or the proxy marker is predictive of a positive therapeutic response in the subject to a treatment with the inhibitor of TL1A activity or expression with a positive predictive value of at least 28%, In some embodiments, the TL1A isoform ratio or the proxy marker is predictive of a positive therapeutic response in the subject to a treatment with the inhibitor of TL1A activity or expression with a positive predictive value of at least 29%, In some embodiments, the TL1A isoform ratio or the proxy marker is predictive of a positive therapeutic response in the subject to a treatment with the inhibitor of TL1A activity or expression with a positive predictive value of at least 30%, In some embodiments, the TL1A isoform ratio or the proxy marker is predictive of a positive therapeutic response in the subject to a treatment with the inhibitor of TL1A activity or expression with a positive predictive value of at least 31%, In some embodiments, the TL1A isoform ratio or the proxy marker is predictive of a positive therapeutic response in the subject to a treatment with the inhibitor of TL1A activity or expression with a positive predictive value of at least 32%, In some embodiments, the TL1A isoform ratio or the proxy marker is predictive of a positive therapeutic response in the subject to a treatment with the inhibitor of TL1A activity or expression with a positive predictive value of at least 33%, In some embodiments, the TL1A isoform ratio or the proxy marker is predictive of a positive therapeutic response in the subject to a treatment with the inhibitor of TL1A activity or expression with a positive predictive value of at least 34%, In some embodiments, the TL1A isoform ratio or the proxy marker is predictive of a positive therapeutic response in the subject to a treatment with the inhibitor of TL1A activity or expression with a positive predictive value of at least 35%, In some embodiments, the TL1A isoform ratio or the proxy marker is predictive of a positive therapeutic response in the subject to a treatment with the inhibitor of TL1A activity or expression with a positive predictive value of at least 40%, In some embodiments, the TL1A isoform ratio or the proxy marker is predictive of a positive therapeutic response in the subject to a treatment with the inhibitor of TL1A activity or expression with a positive predictive value of at least 45%, In some embodiments, the TL1A isoform ratio or the proxy marker is predictive of a positive therapeutic response in the subject to a treatment with the inhibitor of TL1A activity or expression with a positive predictive value of at least 50%, In some embodiments, the TL1A isoform ratio or the proxy marker is predictive of a positive therapeutic response in the subject to a treatment with the inhibitor of TL1A activity or expression with a positive predictive value of at least 51%, In some embodiments, the TL1A isoform ratio or the proxy marker is predictive of a positive therapeutic response in the subject to a treatment with the inhibitor of TL1A activity or expression with a positive predictive value of at least 55%, In some embodiments, the TL1A isoform ratio or the proxy marker is predictive of a positive therapeutic response in the subject to a treatment with the inhibitor of TL1A activity or expression with a positive predictive value of at least 60%, In some embodiments, the TL1A isoform ratio or the proxy marker is predictive of a positive therapeutic response in the subject to a treatment with the inhibitor of TL1A activity or expression with a positive predictive value of at least 65%, In some embodiments, the TL1A isoform ratio or the proxy marker is predictive of a positive therapeutic response in the subject to a treatment with the inhibitor of TL1A activity or expression with a positive predictive value of at least 70%, In some embodiments, the TL1A isoform ratio or the proxy marker is predictive of a positive therapeutic response in the subject to a treatment with the inhibitor of TL1A activity or expression with a positive predictive value of at least 75%, In some embodiments, the TL1A isoform ratio or the proxy marker is predictive of a positive therapeutic response in the subject to a treatment with the inhibitor of TL1A activity or expression with a positive predictive value of at least 80%, In some embodiments, the TL1A isoform ratio or the proxy marker is predictive of a positive therapeutic response in the subject to a treatment with the inhibitor of TL1A activity or expression with a positive predictive value of at least 85%, In some embodiments, the TL1A isoform ratio or the proxy marker is predictive of a positive therapeutic response in the subject to a treatment with the inhibitor of TL1A activity or expression with a positive predictive value of at least 90%, In some embodiments, the TL1A isoform ratio or the proxy marker is predictive of a positive therapeutic response in the subject to a treatment with the inhibitor of TL1A activity or expression with a positive predictive value of at least 95%, In some embodiments, the TL1A isoform ratio or the proxy marker is predictive of a positive therapeutic response in the subject to a treatment with the inhibitor of TL1A activity or expression with a positive predictive value of about 25%, In some embodiments, the TL1A isoform ratio or the proxy marker is predictive of a positive therapeutic response in the subject to a treatment with the inhibitor of TL1A activity or expression with a positive predictive value of about 26%, In some embodiments, the TL1A isoform ratio or the proxy marker is predictive of a positive therapeutic response in the subject to a treatment with the inhibitor of TL1A activity or expression with a positive predictive value of about 27%, In some embodiments, the TL1A isoform ratio or the proxy marker is predictive of a positive therapeutic response in the subject to a treatment with the inhibitor of TL1A activity or expression with a positive predictive value of about 28%, In some embodiments, the TL1A isoform ratio or the proxy marker is predictive of a positive therapeutic response in the subject to a treatment with the inhibitor of TL1A activity or expression with a positive predictive value of about 29%, In some embodiments, the TL1A isoform ratio or the proxy marker is predictive of a positive therapeutic response in the subject to a treatment with the inhibitor of TL1A activity or expression with a positive predictive value of about 30%, In some embodiments, the TL1A isoform ratio or the proxy marker is predictive of a positive therapeutic response in the subject to a treatment with the inhibitor of TL1A activity or expression with a positive predictive value of about 31%, In some embodiments, the TL1A isoform ratio or the proxy marker is predictive of a positive therapeutic response in the subject to a treatment with the inhibitor of TL1A activity or expression with a positive predictive value of about 32%, In some embodiments, the TL1A isoform ratio or the proxy marker is predictive of a positive therapeutic response in the subject to a treatment with the inhibitor of TL1A activity or expression with a positive predictive value of about 33%, In some embodiments, the TL1A isoform ratio or the proxy marker is predictive of a positive therapeutic response in the subject to a treatment with the inhibitor of TL1A activity or expression with a positive predictive value of about 34%, In some embodiments, the TL1A isoform ratio or the proxy marker is predictive of a positive therapeutic response in the subject to a treatment with the inhibitor of TL1A activity or expression with a positive predictive value of about 35%, In some embodiments, the TL1A isoform ratio or the proxy marker is predictive of a positive therapeutic response in the subject to a treatment with the inhibitor of TL1A activity or expression with a positive predictive value of about 40%, In some embodiments, the TL1A isoform ratio or the proxy marker is predictive of a positive therapeutic response in the subject to a treatment with the inhibitor of TL1A activity or expression with a positive predictive value of about 45%, In some embodiments, the TL1A isoform ratio or the proxy marker is predictive of a positive therapeutic response in the subject to a treatment with the inhibitor of TL1A activity or expression with a positive predictive value of about 50%, In some embodiments, the TL1A isoform ratio or the proxy marker is predictive of a positive therapeutic response in the subject to a treatment with the inhibitor of TL1A activity or expression with a positive predictive value of about 51%, In some embodiments, the TL1A isoform ratio or the proxy marker is predictive of a positive therapeutic response in the subject to a treatment with the inhibitor of TL1A activity or expression with a positive predictive value of about 55%, In some embodiments, the TL1A isoform ratio or the proxy marker is predictive of a positive therapeutic response in the subject to a treatment with the inhibitor of TL1A activity or expression with a positive predictive value of about 60%, In some embodiments, the TL1A isoform ratio or the proxy marker is predictive of a positive therapeutic response in the subject to a treatment with the inhibitor of TL1A activity or expression with a positive predictive value of about 65%, In some embodiments, the TL1A isoform ratio or the proxy marker is predictive of a positive therapeutic response in the subject to a treatment with the inhibitor of TL1A activity or expression with a positive predictive value of about 70%, In some embodiments, the TL1A isoform ratio or the proxy marker is predictive of a positive therapeutic response in the subject to a treatment with the inhibitor of TL1A activity or expression with a positive predictive value of about 75%, In some embodiments, the TL1A isoform ratio or the proxy marker is predictive of a positive therapeutic response in the subject to a treatment with the inhibitor of TL1A activity or expression with a positive predictive value of about 80%, In some embodiments, the TL1A isoform ratio or the proxy marker is predictive of a positive therapeutic response in the subject to a treatment with the inhibitor of TL1A activity or expression with a positive predictive value of about 85%, In some embodiments, the TL1A isoform ratio or the proxy marker is predictive of a positive therapeutic response in the subject to a treatment with the inhibitor of TL1A activity or expression with a positive predictive value of about 90%, In some embodiments, the TL1A isoform ratio or the proxy marker is predictive of a positive therapeutic response in the subject to a treatment with the inhibitor of TL1A activity or expression with a positive predictive value of about 95%, In some embodiments, the TL1A isoform ratio or the proxy marker is predictive of a positive therapeutic response in the subject to a treatment with the inhibitor of TL1A activity or expression with a positive predictive value from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 25% to 70%, from 30% to 70%, from 35% to 70%, from 40% to 70%, from 45% to 70%, from 50% to 70%, from 55% to 70%, from 60% to 70%, from 65% to 70%, from 25% to 65%, from 30% to 65%, from 35% to 65%, from 40% to 65%, from 45% to 65%, from 50% to 65%, from 55% to 65%, from 60% to 65%, from 25% to 60%, from 30% to 60%, from 35% to 60%, from 40% to 60%, from 45% to 60%, from 50% to 60%, from 55% to 60%, from 25% to 55%, from 30% to 55%, from 35% to 55%, from 40% to 55%, from 45% to 55%, from 50% to 55%, from 25% to 50%, from 30% to 50%, from 35% to 50%, from 40% to 50%, from 45% to 50%, from 25% to 45%, from 30% to 45%, from 35% to 45%, from 40% to 45%, from 25% to 40%, from 30% to 40%, from 35% to 40%, from 25% to 35%, from 30% to 35%, or from 25% to 30%, As is clear from the description, the element “the TL1A isoform ratio or the proxy marker is predictive of” means that each embodiment described in this paragraph further includes two embodiments with one reciting “the TL1A isoform ratio is predictive of” and the other reciting “the proxy marker is predictive of.” Furthermore, for each embodiment described in this paragraph, the element “the proxy marker is predictive of”′ is (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation. Additionally, for each embodiment described in this paragraph, an additional embodiment is provided wherein the element “the TL1A isoform ratio is predictive of” is “the TL1A isoform ratio above a cutoff is predictive of.”

Accordingly, in various embodiments of the methods provided herein including in Sections 2, 5.1 (e.g. the preceding paragraphs), and 7, the TL1A isoform ratio above the cutoff predicts a positive therapeutic response in the subject to a treatment with the inhibitor of TL1A activity or expression with a positive predictive value of at least 25%, The other embodiments described in this paragraph are provided for the various methods described herein including in Sections 2, 5.1 (e.g. the preceding paragraphs), and 7. In some embodiments, the TL1A isoform ratio above the cutoff predicts a positive therapeutic response in the subject to a treatment with the inhibitor of TL1A activity or expression with a positive predictive value of at least 26%, In some embodiments, the TL1A isoform ratio above the cutoff predicts a positive therapeutic response in the subject to a treatment with the inhibitor of TL1A activity or expression with a positive predictive value of at least 27%, In some embodiments, the TL1A isoform ratio above the cutoff predicts a positive therapeutic response in the subject to a treatment with the inhibitor of TL1A activity or expression with a positive predictive value of at least 28%, In some embodiments, the TL1A isoform ratio above the cutoff predicts a positive therapeutic response in the subject to a treatment with the inhibitor of TL1A activity or expression with a positive predictive value of at least 29%, In some embodiments, the TL1A isoform ratio above the cutoff predicts a positive therapeutic response in the subject to a treatment with the inhibitor of TL1A activity or expression with a positive predictive value of at least 30%, In some embodiments, the TL1A isoform ratio above the cutoff predicts a positive therapeutic response in the subject to a treatment with the inhibitor of TL1A activity or expression with a positive predictive value of at least 31%, In some embodiments, the TL1A isoform ratio above the cutoff predicts a positive therapeutic response in the subject to a treatment with the inhibitor of TL1A activity or expression with a positive predictive value of at least 32%, In some embodiments, the TL1A isoform ratio above the cutoff predicts a positive therapeutic response in the subject to a treatment with the inhibitor of TL1A activity or expression with a positive predictive value of at least 33%, In some embodiments, the TL1A isoform ratio above the cutoff predicts a positive therapeutic response in the subject to a treatment with the inhibitor of TL1A activity or expression with a positive predictive value of at least 34%, In some embodiments, the TL1A isoform ratio above the cutoff predicts a positive therapeutic response in the subject to a treatment with the inhibitor of TL1A activity or expression with a positive predictive value of at least 35%, In some embodiments, the TL1A isoform ratio above the cutoff predicts a positive therapeutic response in the subject to a treatment with the inhibitor of TL1A activity or expression with a positive predictive value of at least 40%, In some embodiments, the TL1A isoform ratio above the cutoff predicts a positive therapeutic response in the subject to a treatment with the inhibitor of TL1A activity or expression with a positive predictive value of at least 45%, In some embodiments, the TL1A isoform ratio above the cutoff predicts a positive therapeutic response in the subject to a treatment with the inhibitor of TL1A activity or expression with a positive predictive value of at least 50%, In some embodiments, the TL1A isoform ratio above the cutoff predicts a positive therapeutic response in the subject to a treatment with the inhibitor of TL1A activity or expression with a positive predictive value of at least 51%, In some embodiments, the TL1A isoform ratio above the cutoff predicts a positive therapeutic response in the subject to a treatment with the inhibitor of TL1A activity or expression with a positive predictive value of at least 55%, In some embodiments, the TL1A isoform ratio above the cutoff predicts a positive therapeutic response in the subject to a treatment with the inhibitor of TL1A activity or expression with a positive predictive value of at least 60%, In some embodiments, the TL1A isoform ratio above the cutoff predicts a positive therapeutic response in the subject to a treatment with the inhibitor of TL1A activity or expression with a positive predictive value of at least 65%, In some embodiments, the TL1A isoform ratio above the cutoff predicts a positive therapeutic response in the subject to a treatment with the inhibitor of TL1A activity or expression with a positive predictive value of at least 70%, In some embodiments, the TL1A isoform ratio above the cutoff predicts a positive therapeutic response in the subject to a treatment with the inhibitor of TL1A activity or expression with a positive predictive value of at least 75%, In some embodiments, the TL1A isoform ratio above the cutoff predicts a positive therapeutic response in the subject to a treatment with the inhibitor of TL1A activity or expression with a positive predictive value of at least 80%, In some embodiments, the TL1A isoform ratio above the cutoff predicts a positive therapeutic response in the subject to a treatment with the inhibitor of TL1A activity or expression with a positive predictive value of at least 85%, In some embodiments, the TL1A isoform ratio above the cutoff predicts a positive therapeutic response in the subject to a treatment with the inhibitor of TL1A activity or expression with a positive predictive value of at least 90%, In some embodiments, the TL1A isoform ratio above the cutoff predicts a positive therapeutic response in the subject to a treatment with the inhibitor of TL1A activity or expression with a positive predictive value of at least 95%, In some embodiments, the TL1A isoform ratio above the cutoff predicts a positive therapeutic response in the subject to a treatment with the inhibitor of TL1A activity or expression with a positive predictive value of about 25%, In some embodiments, the TL1A isoform ratio above the cutoff predicts a positive therapeutic response in the subject to a treatment with the inhibitor of TL1A activity or expression with a positive predictive value of about 26%, In some embodiments, the TL1A isoform ratio above the cutoff predicts a positive therapeutic response in the subject to a treatment with the inhibitor of TL1A activity or expression with a positive predictive value of about 27%, In some embodiments, the TL1A isoform ratio above the cutoff predicts a positive therapeutic response in the subject to a treatment with the inhibitor of TL1A activity or expression with a positive predictive value of about 28%, In some embodiments, the TL1A isoform ratio above the cutoff predicts a positive therapeutic response in the subject to a treatment with the inhibitor of TL1A activity or expression with a positive predictive value of about 29%, In some embodiments, the TL1A isoform ratio above the cutoff predicts a positive therapeutic response in the subject to a treatment with the inhibitor of TL1A activity or expression with a positive predictive value of about 30%, In some embodiments, the TL1A isoform ratio above the cutoff predicts a positive therapeutic response in the subject to a treatment with the inhibitor of TL1A activity or expression with a positive predictive value of about 31%, In some embodiments, the TL1A isoform ratio above the cutoff predicts a positive therapeutic response in the subject to a treatment with the inhibitor of TL1A activity or expression with a positive predictive value of about 32%, In some embodiments, the TL1A isoform ratio above the cutoff predicts a positive therapeutic response in the subject to a treatment with the inhibitor of TL1A activity or expression with a positive predictive value of about 33%, In some embodiments, the TL1A isoform ratio above the cutoff predicts a positive therapeutic response in the subject to a treatment with the inhibitor of TL1A activity or expression with a positive predictive value of about 34%, In some embodiments, the TL1A isoform ratio above the cutoff predicts a positive therapeutic response in the subject to a treatment with the inhibitor of TL1A activity or expression with a positive predictive value of about 35%, In some embodiments, the TL1A isoform ratio above the cutoff predicts a positive therapeutic response in the subject to a treatment with the inhibitor of TL1A activity or expression with a positive predictive value of about 40%, In some embodiments, the TL1A isoform ratio above the cutoff predicts a positive therapeutic response in the subject to a treatment with the inhibitor of TL1A activity or expression with a positive predictive value of about 45%, In some embodiments, the TL1A isoform ratio above the cutoff predicts a positive therapeutic response in the subject to a treatment with the inhibitor of TL1A activity or expression with a positive predictive value of about 50%, In some embodiments, the TL1A isoform ratio above the cutoff predicts a positive therapeutic response in the subject to a treatment with the inhibitor of TL1A activity or expression with a positive predictive value of about 51%, In some embodiments, the TL1A isoform ratio above the cutoff predicts a positive therapeutic response in the subject to a treatment with the inhibitor of TL1A activity or expression with a positive predictive value of about 55%, In some embodiments, the TL1A isoform ratio above the cutoff predicts a positive therapeutic response in the subject to a treatment with the inhibitor of TL1A activity or expression with a positive predictive value of about 60%, In some embodiments, the TL1A isoform ratio above the cutoff predicts a positive therapeutic response in the subject to a treatment with the inhibitor of TL1A activity or expression with a positive predictive value of about 65%, In some embodiments, the TL1A isoform ratio above the cutoff predicts a positive therapeutic response in the subject to a treatment with the inhibitor of TL1A activity or expression with a positive predictive value of about 70%, In some embodiments, the TL1A isoform ratio above the cutoff predicts a positive therapeutic response in the subject to a treatment with the inhibitor of TL1A activity or expression with a positive predictive value of about 75%, In some embodiments, the TL1A isoform ratio above the cutoff predicts a positive therapeutic response in the subject to a treatment with the inhibitor of TL1A activity or expression with a positive predictive value of about 80%, In some embodiments, the TL1A isoform ratio above the cutoff predicts a positive therapeutic response in the subject to a treatment with the inhibitor of TL1A activity or expression with a positive predictive value of about 85%, In some embodiments, the TL1A isoform ratio above the cutoff predicts a positive therapeutic response in the subject to a treatment with the inhibitor of TL1A activity or expression with a positive predictive value of about 90%, In some embodiments, the TL1A isoform ratio above the cutoff predicts a positive therapeutic response in the subject to a treatment with the inhibitor of TL1A activity or expression with a positive predictive value of about 95%, In some embodiments, the TL1A isoform ratio above the cutoff predicts a positive therapeutic response in the subject to a treatment with the inhibitor of TL1A activity or expression with a positive predictive value from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 25% to 70%, from 30% to 70%, from 35% to 70%, from 40% to 70%, from 45% to 70%, from 50% to 70%, from 55% to 70%, from 60% to 70%, from 65% to 70%, from 25% to 65%, from 30% to 65%, from 35% to 65%, from 40% to 65%, from 45% to 65%, from 50% to 65%, from 55% to 65%, from 60% to 65%, from 25% to 60%, from 30% to 60%, from 35% to 60%, from 40% to 60%, from 45% to 60%, from 50% to 60%, from 55% to 60%, from 25% to 55%, from 30% to 55%, from 35% to 55%, from 40% to 55%, from 45% to 55%, from 50% to 55%, from 25% to 50%, from 30% to 50%, from 35% to 50%, from 40% to 50%, from 45% to 50%, from 25% to 45%, from 30% to 45%, from 35% to 45%, from 40% to 45%, from 25% to 40%, from 30% to 40%, from 35% to 40%, from 25% to 35%, from 30% to 35%, or from 25% to 30%, Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Furthermore, for each embodiment described in this paragraph, also provided is a corresponding embodiment wherein the phrase “the TL1A isoform ratio above the cutoff predicts” is substituted by the phrase “the cutoff is such that the proxy marker above the cutoff is predictive of.”

In various embodiments of the methods provided herein including in Sections 2, 5.1 (e.g. the preceding paragraphs), and 7, the TL1A isoform ratio or the proxy marker is predictive of a positive therapeutic response in the subject to a treatment with the inhibitor of TL1A activity or expression with a negative predictive value of at least 40%, The other embodiments described in this paragraph are provided for the various methods described herein including in Sections 2, 5.1 (e.g. the preceding paragraphs), and 7. In some embodiments, the TL1A isoform ratio or the proxy marker is predictive of a positive therapeutic response in the subject to a treatment with the inhibitor of TL1A activity or expression with a negative predictive value of at least 45%, In some embodiments, the TL1A isoform ratio or the proxy marker is predictive of a positive therapeutic response in the subject to a treatment with the inhibitor of TL1A activity or expression with a negative predictive value of at least 50%, In some embodiments, the TL1A isoform ratio or the proxy marker is predictive of a positive therapeutic response in the subject to a treatment with the inhibitor of TL1A activity or expression with a negative predictive value of at least 55%, In some embodiments, the TL1A isoform ratio or the proxy marker is predictive of a positive therapeutic response in the subject to a treatment with the inhibitor of TL1A activity or expression with a negative predictive value of at least 60%, In some embodiments, the TL1A isoform ratio or the proxy marker is predictive of a positive therapeutic response in the subject to a treatment with the inhibitor of TL1A activity or expression with a negative predictive value of at least 65%, In some embodiments, the TL1A isoform ratio or the proxy marker is predictive of a positive therapeutic response in the subject to a treatment with the inhibitor of TL1A activity or expression with a negative predictive value of at least 70%, In some embodiments, the TL1A isoform ratio or the proxy marker is predictive of a positive therapeutic response in the subject to a treatment with the inhibitor of TL1A activity or expression with a negative predictive value of at least 75%, In some embodiments, the TL1A isoform ratio or the proxy marker is predictive of a positive therapeutic response in the subject to a treatment with the inhibitor of TL1A activity or expression with a negative predictive value of at least 80%, In some embodiments, the TL1A isoform ratio or the proxy marker is predictive of a positive therapeutic response in the subject to a treatment with the inhibitor of TL1A activity or expression with a negative predictive value of at least 85%, In some embodiments, the TL1A isoform ratio or the proxy marker is predictive of a positive therapeutic response in the subject to a treatment with the inhibitor of TL1A activity or expression with a negative predictive value of at least 90%, In some embodiments, the TL1A isoform ratio or the proxy marker is predictive of a positive therapeutic response in the subject to a treatment with the inhibitor of TL1A activity or expression with a negative predictive value of at least 95%, In some embodiments, the TL1A isoform ratio or the proxy marker is predictive of a positive therapeutic response in the subject to a treatment with the inhibitor of TL1A activity or expression with a negative predictive value of about 40%, In some embodiments, the TL1A isoform ratio or the proxy marker is predictive of a positive therapeutic response in the subject to a treatment with the inhibitor of TL1A activity or expression with a negative predictive value of about 45%, In some embodiments, the TL1A isoform ratio or the proxy marker is predictive of a positive therapeutic response in the subject to a treatment with the inhibitor of TL1A activity or expression with a negative predictive value of about 50%, In some embodiments, the TL1A isoform ratio or the proxy marker is predictive of a positive therapeutic response in the subject to a treatment with the inhibitor of TL1A activity or expression with a negative predictive value of about 55%, In some embodiments, the TL1A isoform ratio or the proxy marker is predictive of a positive therapeutic response in the subject to a treatment with the inhibitor of TL1A activity or expression with a negative predictive value of about 60%, In some embodiments, the TL1A isoform ratio or the proxy marker is predictive of a positive therapeutic response in the subject to a treatment with the inhibitor of TL1A activity or expression with a negative predictive value of about 65%, In some embodiments, the TL1A isoform ratio or the proxy marker is predictive of a positive therapeutic response in the subject to a treatment with the inhibitor of TL1A activity or expression with a negative predictive value of about 70%, In some embodiments, the TL1A isoform ratio or the proxy marker is predictive of a positive therapeutic response in the subject to a treatment with the inhibitor of TL1A activity or expression with a negative predictive value of about 75%, In some embodiments, the TL1A isoform ratio or the proxy marker is predictive of a positive therapeutic response in the subject to a treatment with the inhibitor of TL1A activity or expression with a negative predictive value of about 80%, In some embodiments, the TL1A isoform ratio or the proxy marker is predictive of a positive therapeutic response in the subject to a treatment with the inhibitor of TL1A activity or expression with a negative predictive value of about 85%, In some embodiments, the TL1A isoform ratio or the proxy marker is predictive of a positive therapeutic response in the subject to a treatment with the inhibitor of TL1A activity or expression with a negative predictive value of about 90%, In some embodiments, the TL1A isoform ratio or the proxy marker is predictive of a positive therapeutic response in the subject to a treatment with the inhibitor of TL1A activity or expression with a negative predictive value of about 95%, In some embodiments, the TL1A isoform ratio or the proxy marker is predictive of a positive therapeutic response in the subject to a treatment with the inhibitor of TL1A activity or expression with a negative predictive value from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%, As is clear from the description, the element “the TL1A isoform ratio or the proxy marker is predictive of” means that each embodiment described in this paragraph further includes two embodiments with one reciting “the TL1A isoform ratio is predictive of” and the other reciting “the proxy marker is predictive of.” Furthermore, for each embodiment described in this paragraph, the element “the proxy marker is predictive of” is (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation. Additionally, for each embodiment described in this paragraph, an additional embodiment is provided wherein the element “the TL1A isoform ratio is predictive of” is “the TL1A isoform ratio above a cutoff is predictive of.”

Accordingly, in various embodiments of the methods provided herein including in Sections 2, 5.1 (e.g. the preceding paragraphs), and 7, the TL1A isoform ratio above the cutoff predicts a positive therapeutic response in the subject to a treatment with the inhibitor of TL1A activity or expression with a negative predictive value of at least 40%, The other embodiments described in this paragraph are provided for the various methods described herein including in Sections 2, 5.1 (e.g. the preceding paragraphs), and 7. In some embodiments, the TL1A isoform ratio above the cutoff predicts a positive therapeutic response in the subject to a treatment with the inhibitor of TL1A activity or expression with a negative predictive value of at least 45%, In some embodiments, the TL1A isoform ratio above the cutoff predicts a positive therapeutic response in the subject to a treatment with the inhibitor of TL1A activity or expression with a negative predictive value of at least 50%, In some embodiments, the TL1A isoform ratio above the cutoff predicts a positive therapeutic response in the subject to a treatment with the inhibitor of TL1A activity or expression with a negative predictive value of at least 55%, In some embodiments, the TL1A isoform ratio above the cutoff predicts a positive therapeutic response in the subject to a treatment with the inhibitor of TL1A activity or expression with a negative predictive value of at least 60%, In some embodiments, the TL1A isoform ratio above the cutoff predicts a positive therapeutic response in the subject to a treatment with the inhibitor of TL1A activity or expression with a negative predictive value of at least 65%, In some embodiments, the TL1A isoform ratio above the cutoff predicts a positive therapeutic response in the subject to a treatment with the inhibitor of TL1A activity or expression with a negative predictive value of at least 70%, In some embodiments, the TL1A isoform ratio above the cutoff predicts a positive therapeutic response in the subject to a treatment with the inhibitor of TL1A activity or expression with a negative predictive value of at least 75%, In some embodiments, the TL1A isoform ratio above the cutoff predicts a positive therapeutic response in the subject to a treatment with the inhibitor of TL1A activity or expression with a negative predictive value of at least 80%, In some embodiments, the TL1A isoform ratio above the cutoff predicts a positive therapeutic response in the subject to a treatment with the inhibitor of TL1A activity or expression with a negative predictive value of at least 85%, In some embodiments, the TL1A isoform ratio above the cutoff predicts a positive therapeutic response in the subject to a treatment with the inhibitor of TL1A activity or expression with a negative predictive value of at least 90%, In some embodiments, the TL1A isoform ratio above the cutoff predicts a positive therapeutic response in the subject to a treatment with the inhibitor of TL1A activity or expression with a negative predictive value of at least 95%, In some embodiments, the TL1A isoform ratio above the cutoff predicts a positive therapeutic response in the subject to a treatment with the inhibitor of TL1A activity or expression with a negative predictive value of about 40%, In some embodiments, the TL1A isoform ratio above the cutoff predicts a positive therapeutic response in the subject to a treatment with the inhibitor of TL1A activity or expression with a negative predictive value of about 45%, In some embodiments, the TL1A isoform ratio above the cutoff predicts a positive therapeutic response in the subject to a treatment with the inhibitor of TL1A activity or expression with a negative predictive value of about 50%, In some embodiments, the TL1A isoform ratio above the cutoff predicts a positive therapeutic response in the subject to a treatment with the inhibitor of TL1A activity or expression with a negative predictive value of about 55%, In some embodiments, the TL1A isoform ratio above the cutoff predicts a positive therapeutic response in the subject to a treatment with the inhibitor of TL1A activity or expression with a negative predictive value of about 60%, In some embodiments, the TL1A isoform ratio above the cutoff predicts a positive therapeutic response in the subject to a treatment with the inhibitor of TL1A activity or expression with a negative predictive value of about 65%, In some embodiments, the TL1A isoform ratio above the cutoff predicts a positive therapeutic response in the subject to a treatment with the inhibitor of TL1A activity or expression with a negative predictive value of about 70%, In some embodiments, the TL1A isoform ratio above the cutoff predicts a positive therapeutic response in the subject to a treatment with the inhibitor of TL1A activity or expression with a negative predictive value of about 75%, In some embodiments, the TL1A isoform ratio above the cutoff predicts a positive therapeutic response in the subject to a treatment with the inhibitor of TL1A activity or expression with a negative predictive value of about 80%, In some embodiments, the TL1A isoform ratio above the cutoff predicts a positive therapeutic response in the subject to a treatment with the inhibitor of TL1A activity or expression with a negative predictive value of about 85%, In some embodiments, the TL1A isoform ratio above the cutoff predicts a positive therapeutic response in the subject to a treatment with the inhibitor of TL1A activity or expression with a negative predictive value of about 90%, In some embodiments, the TL1A isoform ratio above the cutoff predicts a positive therapeutic response in the subject to a treatment with the inhibitor of TL1A activity or expression with a negative predictive value of about 95%, In some embodiments, the TL1A isoform ratio above the cutoff predicts a positive therapeutic response in the subject to a treatment with the inhibitor of TL1A activity or expression with a negative predictive value from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from SS % to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%, Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.”

In various embodiments of the methods provided herein including in Sections 2, 5.1 (e.g. the preceding paragraphs), and 7, the TL1A isoform ratio or the proxy marker is predictive of a positive therapeutic response in the subject to a treatment with the inhibitor of TL1A activity or expression with a sensitivity of at least 20%, The other embodiments described in this paragraph are provided for the various methods described herein including in Sections 2, 5.1 (e.g. the preceding paragraphs), and 7. In some embodiments, the TL1A isoform ratio or the proxy marker is predictive of a positive therapeutic response in the subject to a treatment with the inhibitor of TL1A activity or expression with a sensitivity of at least 25%, In some embodiments, the TL1A isoform ratio or the proxy marker is predictive of a positive therapeutic response in the subject to a treatment with the inhibitor of TL1A activity or expression with a sensitivity of at least 30%, In some embodiments, the TL1A isoform ratio or the proxy marker is predictive of a positive therapeutic response in the subject to a treatment with the inhibitor of TL1A activity or expression with a sensitivity of at least 35%, In some embodiments, the TL1A isoform ratio or the proxy marker is predictive of a positive therapeutic response in the subject to a treatment with the inhibitor of TL1A activity or expression with a sensitivity of at least 40%, In some embodiments, the TL1A isoform ratio or the proxy marker is predictive of a positive therapeutic response in the subject to a treatment with the inhibitor of TL1A activity or expression with a sensitivity of at least 45%, In some embodiments, the TL1A isoform ratio or the proxy marker is predictive of a positive therapeutic response in the subject to a treatment with the inhibitor of TL1A activity or expression with a sensitivity of at least 50%, In some embodiments, the TL1A isoform ratio or the proxy marker is predictive of a positive therapeutic response in the subject to a treatment with the inhibitor of TL1A activity or expression with a sensitivity of at least 55%, In some embodiments, the TL1A isoform ratio or the proxy marker is predictive of a positive therapeutic response in the subject to a treatment with the inhibitor of TL1A activity or expression with a sensitivity of at least 60%, In some embodiments, the TL1A isoform ratio or the proxy marker is predictive of a positive therapeutic response in the subject to a treatment with the inhibitor of TL1A activity or expression with a sensitivity of at least 65%, In some embodiments, the TL1A isoform ratio or the proxy marker is predictive of a positive therapeutic response in the subject to a treatment with the inhibitor of TL1A activity or expression with a sensitivity of at least 70%, In some embodiments, the TL1A isoform ratio or the proxy marker is predictive of a positive therapeutic response in the subject to a treatment with the inhibitor of TL1A activity or expression with a sensitivity of at least 75%, In some embodiments, the TL1A isoform ratio or the proxy marker is predictive of a positive therapeutic response in the subject to a treatment with the inhibitor of TL1A activity or expression with a sensitivity of at least 80%, In some embodiments, the TL1A isoform ratio or the proxy marker is predictive of a positive therapeutic response in the subject to a treatment with the inhibitor of TL1A activity or expression with a sensitivity of at least 85%, In some embodiments, the TL1A isoform ratio or the proxy marker is predictive of a positive therapeutic response in the subject to a treatment with the inhibitor of TL1A activity or expression with a sensitivity of at least 90%, In some embodiments, the TL1A isoform ratio or the proxy marker is predictive of a positive therapeutic response in the subject to a treatment with the inhibitor of TL1A activity or expression with a sensitivity of at least 95%, In some embodiments, the TL1A isoform ratio or the proxy marker is predictive of a positive therapeutic response in the subject to a treatment with the inhibitor of TL1A activity or expression with a sensitivity of about 20%, In some embodiments, the TL1A isoform ratio or the proxy marker is predictive of a positive therapeutic response in the subject to a treatment with the inhibitor of TL1A activity or expression with a sensitivity of about 25%, In some embodiments, the TL1A isoform ratio or the proxy marker is predictive of a positive therapeutic response in the subject to a treatment with the inhibitor of TL1A activity or expression with a sensitivity of about 30%, In some embodiments, the TL1A isoform ratio or the proxy marker is predictive of a positive therapeutic response in the subject to a treatment with the inhibitor of TL1A activity or expression with a sensitivity of about 35%, In some embodiments, the TL1A isoform ratio or the proxy marker is predictive of a positive therapeutic response in the subject to a treatment with the inhibitor of TL1A activity or expression with a sensitivity of about 40%, In some embodiments, the TL1A isoform ratio or the proxy marker is predictive of a positive therapeutic response in the subject to a treatment with the inhibitor of TL1A activity or expression with a sensitivity of about 45%, In some embodiments, the TL1A isoform ratio or the proxy marker is predictive of a positive therapeutic response in the subject to a treatment with the inhibitor of TL1A activity or expression with a sensitivity of about 50%, In some embodiments, the TL1A isoform ratio or the proxy marker is predictive of a positive therapeutic response in the subject to a treatment with the inhibitor of TL1A activity or expression with a sensitivity of about 55%, In some embodiments, the TL1A isoform ratio or the proxy marker is predictive of a positive therapeutic response in the subject to a treatment with the inhibitor of TL1A activity or expression with a sensitivity of about 60%, In some embodiments, the TL1A isoform ratio or the proxy marker is predictive of a positive therapeutic response in the subject to a treatment with the inhibitor of TL1A activity or expression with a sensitivity of about 65%, In some embodiments, the TL1A isoform ratio or the proxy marker is predictive of a positive therapeutic response in the subject to a treatment with the inhibitor of TL1A activity or expression with a sensitivity of about 70%, In some embodiments, the TL1A isoform ratio or the proxy marker is predictive of a positive therapeutic response in the subject to a treatment with the inhibitor of TL1A activity or expression with a sensitivity of about 75%, In some embodiments, the TL1A isoform ratio or the proxy marker is predictive of a positive therapeutic response in the subject to a treatment with the inhibitor of TL1A activity or expression with a sensitivity of about 80%, In some embodiments, the TL1A isoform ratio or the proxy marker is predictive of a positive therapeutic response in the subject to a treatment with the inhibitor of TL1A activity or expression with a sensitivity of about 85%, In some embodiments, the TL1A isoform ratio or the proxy marker is predictive of a positive therapeutic response in the subject to a treatment with the inhibitor of TL1A activity or expression with a sensitivity of about 90%, In some embodiments, the TL1A isoform ratio or the proxy marker is predictive of a positive therapeutic response in the subject to a treatment with the inhibitor of TL1A activity or expression with a sensitivity of about 95%, In some embodiments, the TL1A isoform ratio or the proxy marker is predictive of a positive therapeutic response in the subject to a treatment with the inhibitor of TL1A activity or expression with a sensitivity from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 20% to 85%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 20% to 65%, from 25% to 65%, from 30% to 65%, from 35% to 65%, from 40% to 65%, from 45% to 65%, from 50% to 65%, from 55% to 65%, from 60% to 65%, from 20% to 60%, from 25% to 60%, from 30% to 60%, from 35% to 60%, from 40% to 60%, from 45% to 60%, from 50% to 60%, from 55% to 60%, from 20% to 55%, from 25% to 55%, from 30% to 55%, from 35% to 55%, from 40% to 55%, from 45% to 55%, from 50% to 55%, from 20% to 50%, from 25% to 50%, from 30% to 50%, from 35% to 50%, from 40% to 50%, from 45% to 50%, from 20% to 45%, from 25% to 45%, from 30% to 45%, from 35% to 45%, from 40% to 45%, from 20% to 40%, from 25% to 40%, from 30% to 40%, from 35% to 40%, from 20% to 35%, from 25% to 35%, from 30% to 35%, from 20% to 30%, from 25% to 30%, or from 20% to 25%, As is clear from the description, the element “the TL1A isoform ratio or the proxy marker is predictive of” means that each embodiment described in this paragraph further includes two embodiments with one reciting “the TL1A isoform ratio is predictive of” and the other reciting “the proxy marker is predictive of.” Furthermore, for each embodiment described in this paragraph, the element “the proxy marker is predictive of” is (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation. Additionally, for each embodiment described in this paragraph, an additional embodiment is provided wherein the element “the TL1A isoform ratio is predictive of” is “the TL1A isoform ratio above a cutoff is predictive of.”

Accordingly, in various embodiments of the methods provided herein including in Sections 2, 5.1 (e.g. the preceding paragraphs), and 7, the TL1A isoform ratio above the cutoff predicts a positive therapeutic response in the subject to a treatment with the inhibitor of TL1A activity or expression with a sensitivity of at least 20%, The other embodiments described in this paragraph are provided for the various methods described herein including in Sections 2, 5.1 (e.g. the preceding paragraphs), and 7. In some embodiments, the TL1A isoform ratio above the cutoff predicts a positive therapeutic response in the subject to a treatment with the inhibitor of TL1A activity or expression with a sensitivity of at least 25%, In some embodiments, the TL1A isoform ratio above the cutoff predicts a positive therapeutic response in the subject to a treatment with the inhibitor of TL1A activity or expression with a sensitivity of at least 30%, In some embodiments, the TL1A isoform ratio above the cutoff predicts a positive therapeutic response in the subject to a treatment with the inhibitor of TL1A activity or expression with a sensitivity of at least 35%, In some embodiments, the TL1A isoform ratio above the cutoff predicts a positive therapeutic response in the subject to a treatment with the inhibitor of TL1A activity or expression with a sensitivity of at least 40%, In some embodiments, the TL1A isoform ratio above the cutoff predicts a positive therapeutic response in the subject to a treatment with the inhibitor of TL1A activity or expression with a sensitivity of at least 45%, In some embodiments, the TL1A isoform ratio above the cutoff predicts a positive therapeutic response in the subject to a treatment with the inhibitor of TL1A activity or expression with a sensitivity of at least 50%, In some embodiments, the TL1A isoform ratio above the cutoff predicts a positive therapeutic response in the subject to a treatment with the inhibitor of TL1A activity or expression with a sensitivity of at least 55%, In some embodiments, the TL1A isoform ratio above the cutoff predicts a positive therapeutic response in the subject to a treatment with the inhibitor of TL1A activity or expression with a sensitivity of at least 60%, In some embodiments, the TL1A isoform ratio above the cutoff predicts a positive therapeutic response in the subject to a treatment with the inhibitor of TL1A activity or expression with a sensitivity of at least 65%, In some embodiments, the TL1A isoform ratio above the cutoff predicts a positive therapeutic response in the subject to a treatment with the inhibitor of TL1A activity or expression with a sensitivity of at least 70%, In some embodiments, the TL1A isoform ratio above the cutoff predicts a positive therapeutic response in the subject to a treatment with the inhibitor of TL1A activity or expression with a sensitivity of at least 75%, In some embodiments, the TL1A isoform ratio above the cutoff predicts a positive therapeutic response in the subject to a treatment with the inhibitor of TL1A activity or expression with a sensitivity of at least 80%, In some embodiments, the TL1A isoform ratio above the cutoff predicts a positive therapeutic response in the subject to a treatment with the inhibitor of TL1A activity or expression with a sensitivity of at least 85%, In some embodiments, the TL1A isoform ratio above the cutoff predicts a positive therapeutic response in the subject to a treatment with the inhibitor of TL1A activity or expression with a sensitivity of at least 90%, In some embodiments, the TL1A isoform ratio above the cutoff predicts a positive therapeutic response in the subject to a treatment with the inhibitor of TL1A activity or expression with a sensitivity of at least 95%, In some embodiments, the TL1A isoform ratio above the cutoff predicts a positive therapeutic response in the subject to a treatment with the inhibitor of TL1A activity or expression with a sensitivity of about 20%, In some embodiments, the TL1A isoform ratio above the cutoff predicts a positive therapeutic response in the subject to a treatment with the inhibitor of TL1A activity or expression with a sensitivity of about 25%, In some embodiments, the TL1A isoform ratio above the cutoff predicts a positive therapeutic response in the subject to a treatment with the inhibitor of TL1A activity or expression with a sensitivity of about 30%, In some embodiments, the TL1A isoform ratio above the cutoff predicts a positive therapeutic response in the subject to a treatment with the inhibitor of TL1A activity or expression with a sensitivity of about 35%, In some embodiments, the TL1A isoform ratio above the cutoff predicts a positive therapeutic response in the subject to a treatment with the inhibitor of TL1A activity or expression with a sensitivity of about 40%, In some embodiments, the TL1A isoform ratio above the cutoff predicts a positive therapeutic response in the subject to a treatment with the inhibitor of TL1A activity or expression with a sensitivity of about 45%, In some embodiments, the TL1A isoform ratio above the cutoff predicts a positive therapeutic response in the subject to a treatment with the inhibitor of TL1A activity or expression with a sensitivity of about 50%, In some embodiments, the TL1A isoform ratio above the cutoff predicts a positive therapeutic response in the subject to a treatment with the inhibitor of TL1A activity or expression with a sensitivity of about 55%, In some embodiments, the TL1A isoform ratio above the cutoff predicts a positive therapeutic response in the subject to a treatment with the inhibitor of TL1A activity or expression with a sensitivity of about 60%, In some embodiments, the TL1A isoform ratio above the cutoff predicts a positive therapeutic response in the subject to a treatment with the inhibitor of TL1A activity or expression with a sensitivity of about 65%, In some embodiments, the TL1A isoform ratio above the cutoff predicts a positive therapeutic response in the subject to a treatment with the inhibitor of TL1A activity or expression with a sensitivity of about 70%, In some embodiments, the TL1A isoform ratio above the cutoff predicts a positive therapeutic response in the subject to a treatment with the inhibitor of TL1A activity or expression with a sensitivity of about 75%, In some embodiments, the TL1A isoform ratio above the cutoff predicts a positive therapeutic response in the subject to a treatment with the inhibitor of TL1A activity or expression with a sensitivity of about 80%, In some embodiments, the TL1A isoform ratio above the cutoff predicts a positive therapeutic response in the subject to a treatment with the inhibitor of TL1A activity or expression with a sensitivity of about 85%, In some embodiments, the TL1A isoform ratio above the cutoff predicts a positive therapeutic response in the subject to a treatment with the inhibitor of TL1A activity or expression with a sensitivity of about 90%, In some embodiments, the TL1A isoform ratio above the cutoff predicts a positive therapeutic response in the subject to a treatment with the inhibitor of TL1A activity or expression with a sensitivity of about 95%, In some embodiments, the TL1A isoform ratio above the cutoff predicts a positive therapeutic response in the subject to a treatment with the inhibitor of TL1A activity or expression with a sensitivity from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 20% to 85%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 20% to 65%, from 25% to 65%, from 30% to 65%, from 35% to 65%, from 40% to 65%, from 45% to 65%, from 50% to 65%, from 55% to 65%, from 60% to 65%, from 20% to 60%, from 25% to 60%, from 30% to 60%, from 35% to 60%, from 40% to 60%, from 45% to 60%, from 50% to 60%, from 55% to 60%, from 20% to 55%, from 25% to 55%, from 30% to 55%, from 35% to 55%, from 40% to 55%, from 45% to 55%, from 50% to 55%, from 20% to 50%, from 25% to 50%, from 30% to 50%, from 35% to 50%, from 40% to 50%, from 45% to 50%, from 20% to 45%, from 25% to 45%, from 30% to 45%, from 35% to 45%, from 40% to 45%, from 20% to 40%, from 25% to 40%, from 30% to 40%, from 35% to 40%, from 20% to 35%, from 25% to 35%, from 30% to 35%, from 20% to 30%, from 25% to 30%, or from 20% to 25%, Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.”

In various embodiments of the methods provided herein including in Sections 2, 5.1 (e.g. the preceding paragraphs), and 7, the TL1A isoform ratio or the proxy marker is predictive of a positive therapeutic response in the subject to a treatment with the inhibitor of TL1A activity or expression with a specificity of at least 40%, The other embodiments described in this paragraph are provided for the various methods described herein including in Sections 2, 5.1 (e.g. the preceding paragraphs), and 7. In some embodiments, the TL1A isoform ratio or the proxy marker is predictive of a positive therapeutic response in the subject to a treatment with the inhibitor of TL1A activity or expression with a specificity of at least 45%, In some embodiments, the TL1A isoform ratio or the proxy marker is predictive of a positive therapeutic response in the subject to a treatment with the inhibitor of TL1A activity or expression with a specificity of at least 50%, In some embodiments, the TL1A isoform ratio or the proxy marker is predictive of a positive therapeutic response in the subject to a treatment with the inhibitor of TL1A activity or expression with a specificity of at least 55%, In some embodiments, the TL1A isoform ratio or the proxy marker is predictive of a positive therapeutic response in the subject to a treatment with the inhibitor of TL1A activity or expression with a specificity of at least 60%, In some embodiments, the TL1A isoform ratio or the proxy marker is predictive of a positive therapeutic response in the subject to a treatment with the inhibitor of TL1A activity or expression with a specificity of at least 65%, In some embodiments, the TL1A isoform ratio or the proxy marker is predictive of a positive therapeutic response in the subject to a treatment with the inhibitor of TL1A activity or expression with a specificity of at least 70%, In some embodiments, the TL1A isoform ratio or the proxy marker is predictive of a positive therapeutic response in the subject to a treatment with the inhibitor of TL1A activity or expression with a specificity of at least 75%, In some embodiments, the TL1A isoform ratio or the proxy marker is predictive of a positive therapeutic response in the subject to a treatment with the inhibitor of TL1A activity or expression with a specificity of at least 80%, In some embodiments, the TL1A isoform ratio or the proxy marker is predictive of a positive therapeutic response in the subject to a treatment with the inhibitor of TL1A activity or expression with a specificity of at least 85%, In some embodiments, the TL1A isoform ratio or the proxy marker is predictive of a positive therapeutic response in the subject to a treatment with the inhibitor of TL1A activity or expression with a specificity of at least 90%, In some embodiments, the TL1A isoform ratio or the proxy marker is predictive of a positive therapeutic response in the subject to a treatment with the inhibitor of TL1A activity or expression with a specificity of at least 95%, In some embodiments, the TL1A isoform ratio or the proxy marker is predictive of a positive therapeutic response in the subject to a treatment with the inhibitor of TL1A activity or expression with a specificity of about 40%, In some embodiments, the TL1A isoform ratio or the proxy marker is predictive of a positive therapeutic response in the subject to a treatment with the inhibitor of TL1A activity or expression with a specificity of about 45%, In some embodiments, the TL1A isoform ratio or the proxy marker is predictive of a positive therapeutic response in the subject to a treatment with the inhibitor of TL1A activity or expression with a specificity of about 50%, In some embodiments, the TL1A isoform ratio or the proxy marker is predictive of a positive therapeutic response in the subject to a treatment with the inhibitor of TL1A activity or expression with a specificity of about 55%, In some embodiments, the TL1A isoform ratio or the proxy marker is predictive of a positive therapeutic response in the subject to a treatment with the inhibitor of TL1A activity or expression with a specificity of about 60%, In some embodiments, the TL1A isoform ratio or the proxy marker is predictive of a positive therapeutic response in the subject to a treatment with the inhibitor of TL1A activity or expression with a specificity of about 65%, In some embodiments, the TL1A isoform ratio or the proxy marker is predictive of a positive therapeutic response in the subject to a treatment with the inhibitor of TL1A activity or expression with a specificity of about 70%, In some embodiments, the TL1A isoform ratio or the proxy marker is predictive of a positive therapeutic response in the subject to a treatment with the inhibitor of TL1A activity or expression with a specificity of about 75%, In some embodiments, the TL1A isoform ratio or the proxy marker is predictive of a positive therapeutic response in the subject to a treatment with the inhibitor of TL1A activity or expression with a specificity of about 80%, In some embodiments, the TL1A isoform ratio or the proxy marker is predictive of a positive therapeutic response in the subject to a treatment with the inhibitor of TL1A activity or expression with a specificity of about 85%, In some embodiments, the TL1A isoform ratio or the proxy marker is predictive of a positive therapeutic response in the subject to a treatment with the inhibitor of TL1A activity or expression with a specificity of about 90%, In some embodiments, the TL1A isoform ratio or the proxy marker is predictive of a positive therapeutic response in the subject to a treatment with the inhibitor of TL1A activity or expression with a specificity of about 95%, In some embodiments, the TL1A isoform ratio or the proxy marker is predictive of a positive therapeutic response in the subject to a treatment with the inhibitor of TL1A activity or expression with a specificity from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%, As is clear from the description, the element “the TL1A isoform ratio or the proxy marker is predictive of” means that each embodiment described in this paragraph further includes two embodiments with one reciting “the TL1A isoform ratio is predictive of” and the other reciting “the proxy marker is predictive of.” Furthermore, for each embodiment described in this paragraph, the element “the proxy marker is predictive of” is (1) “the proxy marker above a cutoff is predictive of”′ if the proxy marker and the TL1A isoform ratio has a positive correlation, or (ii) “the proxy marker below a cutoff is predictive of” if the proxy marker and the TL1A isoform ratio has a negative correlation. Additionally, for each embodiment described in this paragraph, an additional embodiment is provided wherein the element “the TL1A isoform ratio is predictive of” is “the TL1A isoform ratio above a cutoff is predictive of.”

Accordingly, in various embodiments of the methods provided herein including in Sections 2, 5.1 (e.g. the preceding paragraphs), and 7, the TL1A isoform ratio above the cutoff predicts a positive therapeutic response in the subject to a treatment with the inhibitor of TL1A activity or expression with a specificity of at least 40%, The other embodiments described in this paragraph are provided for the various methods described herein including in Sections 2, 5.1 (e.g. the preceding paragraphs), and 7. In some embodiments, the TL1A isoform ratio above the cutoff predicts a positive therapeutic response in the subject to a treatment with the inhibitor of TL1A activity or expression with a specificity of at least 45%, In some embodiments, the TL1A isoform ratio above the cutoff predicts a positive therapeutic response in the subject to a treatment with the inhibitor of TL1A activity or expression with a specificity of at least 50%, In some embodiments, the TL1A isoform ratio above the cutoff predicts a positive therapeutic response in the subject to a treatment with the inhibitor of TL1A activity or expression with a specificity of at least 55%, In some embodiments, the TL1A isoform ratio above the cutoff predicts a positive therapeutic response in the subject to a treatment with the inhibitor of TL1A activity or expression with a specificity of at least 60%, In some embodiments, the TL1A isoform ratio above the cutoff predicts a positive therapeutic response in the subject to a treatment with the inhibitor of TL1A activity or expression with a specificity of at least 65%, In some embodiments, the TL1A isoform ratio above the cutoff predicts a positive therapeutic response in the subject to a treatment with the inhibitor of TL1A activity or expression with a specificity of at least 70%, In some embodiments, the TL1A isoform ratio above the cutoff predicts a positive therapeutic response in the subject to a treatment with the inhibitor of TL1A activity or expression with a specificity of at least 75%, In some embodiments, the TL1A isoform ratio above the cutoff predicts a positive therapeutic response in the subject to a treatment with the inhibitor of TL1A activity or expression with a specificity of at least 80%, In some embodiments, the TL1A isoform ratio above the cutoff predicts a positive therapeutic response in the subject to a treatment with the inhibitor of TL1A activity or expression with a specificity of at least 85%, In some embodiments, the TL1A isoform ratio above the cutoff predicts a positive therapeutic response in the subject to a treatment with the inhibitor of TL1A activity or expression with a specificity of at least 90%, In some embodiments, the TL1A isoform ratio above the cutoff predicts a positive therapeutic response in the subject to a treatment with the inhibitor of TL1A activity or expression with a specificity of at least 95%, In some embodiments, the TL1A isoform ratio above the cutoff predicts a positive therapeutic response in the subject to a treatment with the inhibitor of TL1A activity or expression with a specificity of about 40%, In some embodiments, the TL1A isoform ratio above the cutoff predicts a positive therapeutic response in the subject to a treatment with the inhibitor of TL1A activity or expression with a specificity of about 45%, In some embodiments, the TL1A isoform ratio above the cutoff predicts a positive therapeutic response in the subject to a treatment with the inhibitor of TL1A activity or expression with a specificity of about 50%, In some embodiments, the TL1A isoform ratio above the cutoff predicts a positive therapeutic response in the subject to a treatment with the inhibitor of TL1A activity or expression with a specificity of about 55%, In some embodiments, the TL1A isoform ratio above the cutoff predicts a positive therapeutic response in the subject to a treatment with the inhibitor of TL1A activity or expression with a specificity of about 60%, In some embodiments, the TL1A isoform ratio above the cutoff predicts a positive therapeutic response in the subject to a treatment with the inhibitor of TL1A activity or expression with a specificity of about 65%, In some embodiments, the TL1A isoform ratio above the cutoff predicts a positive therapeutic response in the subject to a treatment with the inhibitor of TL1A activity or expression with a specificity of about 70%, In some embodiments, the TL1A isoform ratio above the cutoff predicts a positive therapeutic response in the subject to a treatment with the inhibitor of TL1A activity or expression with a specificity of about 75%, In some embodiments, the TL1A isoform ratio above the cutoff predicts a positive therapeutic response in the subject to a treatment with the inhibitor of TL1A activity or expression with a specificity of about 80%, In some embodiments, the TL1A isoform ratio above the cutoff predicts a positive therapeutic response in the subject to a treatment with the inhibitor of TL1A activity or expression with a specificity of about 85%, In some embodiments, the TL1A isoform ratio above the cutoff predicts a positive therapeutic response in the subject to a treatment with the inhibitor of TL1A activity or expression with a specificity of about 90%, In some embodiments, the TL1A isoform ratio above the cutoff predicts a positive therapeutic response in the subject to a treatment with the inhibitor of TL1A activity or expression with a specificity of about 95%, In some embodiments, the TL1A isoform ratio above the cutoff predicts a positive therapeutic response in the subject to a treatment with the inhibitor of TL1A activity or expression with a specificity from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%, Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.”

In various embodiments of the methods provided herein including in Sections 2, 5.1 (e.g. the preceding paragraphs), and 7, the positive rate of the subjects with the TL1A isoform ratio or the proxy marker as provided in the methods is at least 10%, The other embodiments described in this paragraph are provided for the various methods described herein including in Sections 2, 5.1 (e.g. the preceding paragraphs), and 7. In some embodiments, the positive rate of the subjects with the TL1A isoform ratio or the proxy marker as provided in the methods is at least 15%, In some embodiments, the positive rate of the subjects with the TL1A isoform ratio or the proxy marker as provided in the methods is at least 20%, In some embodiments, the positive rate of the subjects with the TL1A isoform ratio or the proxy marker as provided in the methods is at least 25%, In some embodiments, the positive rate of the subjects with the TL1A isoform ratio or the proxy marker as provided in the methods is at least 30%, In some embodiments, the positive rate of the subjects with the TL1A isoform ratio or the proxy marker as provided in the methods is at least 35%, In some embodiments, the positive rate of the subjects with the TL1A isoform ratio or the proxy marker as provided in the methods is at least 40%, In some embodiments, the positive rate of the subjects with the TL1A isoform ratio or the proxy marker as provided in the methods is at least 45%, In some embodiments, the positive rate of the subjects with the TL1A isoform ratio or the proxy marker as provided in the methods is at least 50%, In some embodiments, the positive rate of the subjects with the TL1A isoform ratio or the proxy marker as provided in the methods is at least 55%, In some embodiments, the positive rate of the subjects with the TL1A isoform ratio or the proxy marker as provided in the methods is at least 60%, In some embodiments, the positive rate of the subjects with the TL1A isoform ratio or the proxy marker as provided in the methods is at least 65%, In some embodiments, the positive rate of the subjects with the TL1A isoform ratio or the proxy marker as provided in the methods is at least 70%, In some embodiments, the positive rate of the subjects with the TL1A isoform ratio or the proxy marker as provided in the methods is at least 75%, In some embodiments, the positive rate of the subjects with the TL1A isoform ratio or the proxy marker as provided in the methods is about 10%, In some embodiments, the positive rate of the subjects with the TL1A isoform ratio or the proxy marker as provided in the methods is about 15%, In some embodiments, the positive rate of the subjects with the TL1A isoform ratio or the proxy marker as provided in the methods is about 20%, In some embodiments, the positive rate of the subjects with the TL1A isoform ratio or the proxy marker as provided in the methods is about 25%, In some embodiments, the positive rate of the subjects with the TL1A isoform ratio or the proxy marker as provided in the methods is about 30%, In some embodiments, the positive rate of the subjects with the TL1A isoform ratio or the proxy marker as provided in the methods is about 35%, In some embodiments, the positive rate of the subjects with the TL1A isoform ratio or the proxy marker as provided in the methods is about 40%, In some embodiments, the positive rate of the subjects with the TL1A isoform ratio or the proxy marker as provided in the methods is about 45%, In some embodiments, the positive rate of the subjects with the TL1A isoform ratio or the proxy marker as provided in the methods is about 50%, In some embodiments, the positive rate of the subjects with the TL1A isoform ratio or the proxy marker as provided in the methods is about 55%, In some embodiments, the positive rate of the subjects with the TL1A isoform ratio or the proxy marker as provided in the methods is about 60%, In some embodiments, the positive rate of the subjects with the TL1A isoform ratio or the proxy marker as provided in the methods is about 65%, In some embodiments, the positive rate of the subjects with the TL1A isoform ratio or the proxy marker as provided in the methods is about 70%, In some embodiments, the positive rate of the subjects with the TL1A isoform ratio or the proxy marker as provided in the methods is about 75%, In some embodiments, the positive rate of the subjects with the TL1A isoform ratio or the proxy marker as provided in the methods is from 10% to 75%, from 15% to 75%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, 10% to 70%, from 15% to 70%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 10% to 65%, from 15% to 65%, from 20% to 65%, from 25% to 65%, from 30% to 65%, from 35% to 65%, from 40% to 65%, from 45% to 65%, from 50% to 65%, from 55% to 65%, from 60% to 65%, from 10% to 60%, from 15% to 60%, from 20% to 60%, from 25% to 60%, from 30% to 60%, from 35% to 60%, from 40% to 60%, from 45% to 60%, from 50% to 60%, from 55% to 60%, from 10% to 55%, from 15% to 55%, from 20% to 55%, from 25% to 55%, from 30% to 55%, from 35% to 55%, from 40% to 55%, from 45% to 55%, from 50% to 55%, from 10% to 50%, from 15% to 50%, from 20% to 50%, from 25% to 50%, from 30% to 50%, from 35% to 50%, from 40% to 50%, from 45% to 50%, from 10% to 45%, from 15% to 45%, from 20% to 45%, from 25% to 45%, from 30% to 45%, from 35% to 45%, from 40% to 45%, from 10% to 40%, from 15% to 40%, from 20% to 40%, from 25% to 40%, from 30% to 40%, from 35% to 40%, from 10% to 35%, from 15% to 35%, from 20% to 35%, from 25% to 35%, from 30% to 35%, from 10% to 30%, from 15% to 30%, from 20% to 30%, from 25% to 30%, from 10% to 25%, from 15% to 25%, from 20% to 25%, from 10% to 20%, from 15% to 20%, or from 10% to 15%, In some specific embodiments, the positive rate of the subjects with the TL1A isoform ratio or the proxy marker as provided in the methods is from 30% to 40%, As is clear from the description, the element “with the TL1A isoform ratio or the proxy marker” means that each embodiment described in this paragraph further includes two embodiments with one reciting “with the TL1A isoform ratio” and the other reciting “with the proxy marker.” Furthermore, for each embodiment described in this paragraph, the element “with the proxy marker” is (1) “with the proxy marker above a cutoff” if the proxy marker and the TL1A isoform ratio has a positive correlation, or (ii) “with the proxy marker below a cutoff” if the proxy marker and the TL1A isoform ratio has a negative correlation. Additionally, for each embodiment described in this paragraph, an additional embodiment is provided wherein the element “with the TL1A isoform ratio” is “with the TL1A isoform ratio above a cutoff.”

Accordingly, in various embodiments of the methods provided herein including in Sections 2, 5.1 (e.g. the preceding paragraphs), and 7, the positive rate of the subjects with the TL1A isoform ratio above the cutoff as provided in the methods is at least 10%, The other embodiments described in this paragraph are provided for the various methods described herein including in Sections 2, 5.1 (e.g. the preceding paragraphs), and 7. In some embodiments, the positive rate of the subjects with the TL1A isoform ratio above the cutoff as provided in the methods is at least 15%, In some embodiments, the positive rate of the subjects with the TL1A isoform ratio above the cutoff as provided in the methods is at least 20%, In some embodiments, the positive rate of the subjects with the TL1A isoform ratio above the cutoff as provided in the methods is at least 25%, In some embodiments, the positive rate of the subjects with the TL1A isoform ratio above the cutoff as provided in the methods is at least 30%, In some embodiments, the positive rate of the subjects with the TL1A isoform ratio above the cutoff as provided in the methods is at least 35%, In some embodiments, the positive rate of the subjects with the TL1A isoform ratio above the cutoff as provided in the methods is at least 40%, In some embodiments, the positive rate of the subjects with the TL1A isoform ratio above the cutoff as provided in the methods is at least 45%, In some embodiments, the positive rate of the subjects with the TL1A isoform ratio above the cutoff as provided in the methods is at least 50%, In some embodiments, the positive rate of the subjects with the TL1A isoform ratio above the cutoff as provided in the methods is at least 55%, In some embodiments, the positive rate of the subjects with the TL1A isoform ratio above the cutoff as provided in the methods is at least 60%, In some embodiments, the positive rate of the subjects with the TL1A isoform ratio above the cutoff as provided in the methods is at least 65%, In some embodiments, the positive rate of the subjects with the TL1A isoform ratio above the cutoff as provided in the methods is at least 70%, In some embodiments, the positive rate of the subjects with the TL1A isoform ratio above the cutoff as provided in the methods is at least 75%, In some embodiments, the positive rate of the subjects with the TL1A isoform ratio above the cutoff as provided in the methods is about 10%, In some embodiments, the positive rate of the subjects with the TL1A isoform ratio above the cutoff as provided in the methods is about 15%, In some embodiments, the positive rate of the subjects with the TL1A isoform ratio above the cutoff as provided in the methods is about 20%, In some embodiments, the positive rate of the subjects with the TL1A isoform ratio above the cutoff as provided in the methods is about 25%, In some embodiments, the positive rate of the subjects with the TL1A isoform ratio above the cutoff as provided in the methods is about 30%, In some embodiments, the positive rate of the subjects with the TL1A isoform ratio above the cutoff as provided in the methods is about 35%, In some embodiments, the positive rate of the subjects with the TL1A isoform ratio above the cutoff as provided in the methods is about 40%, In some embodiments, the positive rate of the subjects with the TL1A isoform ratio above the cutoff as provided in the methods is about 45%, In some embodiments, the positive rate of the subjects with the TL1A isoform ratio above the cutoff as provided in the methods is about 50%, In some embodiments, the positive rate of the subjects with the TL1A isoform ratio above the cutoff as provided in the methods is about 55%, In some embodiments, the positive rate of the subjects with the TL1A isoform ratio above the cutoff as provided in the methods is about 60%, In some embodiments, the positive rate of the subjects with the TL1A isoform ratio above the cutoff as provided in the methods is about 65%, In some embodiments, the positive rate of the subjects with the TL1A isoform ratio above the cutoff as provided in the methods is about 70%, In some embodiments, the positive rate of the subjects with the TL1A isoform ratio above the cutoff as provided in the methods is about 75%, In some embodiments, the positive rate of the subjects with the TL1A isoform ratio above the cutoff as provided in the methods is from 10% to 75%, from 15% to 75%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, 10% to 70%, from 15% to 70%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 10% to 65%, from 15% to 65%, from 20% to 65%, from 25% to 65%, from 30% to 65%, from 35% to 65%, from 40% to 65%, from 45% to 65%, from 50% to 65%, from 55% to 65%, from 60% to 65%, from 10% to 60%, from 15% to 60%, from 20% to 60%, from 25% to 60%, from 30% to 60%, from 35% to 60%, from 40% to 60%, from 45% to 60%, from 50% to 60%, from 55% to 60%, from 10% to 55%, from 15% to 55%, from 20% to 55%, from 25% to 55%, from 30% to 55%, from 35% to 55%, from 40% to 55%, from 45% to 55%, from 50% to 55%, from 10% to 50%, from 15% to 50%, from 20% to 50%, from 25% to 50%, from 30% to 50%, from 35% to 50%, from 40% to 50%, from 45% to 50%, from 10% to 45%, from 15% to 45%, from 20% to 45%, from 25% to 45%, from 30% to 45%, from 35% to 45%, from 40% to 45%, from 10% to 40%, from 15% to 40%, from 20% to 40%, from 25% to 40%, from 30% to 40%, from 35% to 40%, from 10% to 35%, from 15% to 35%, from 20% to 35%, from 25% to 35%, from 30% to 35%, from 10% to 30%, from 15% to 30%, from 20% to 30%, from 25% to 30%, from 10% to 25%, from 15% to 25%, from 20% to 25%, from 10% to 20%, from 15% to 20%, or from 10% to 15%, In some specific embodiments, the positive rate of the subjects with the TL1A isoform ratio above the cutoff as provided in the methods is from 30% to 40%, Furthermore, for each embodiment described in this paragraph, also provided is a corresponding embodiment wherein the phrase “the positive rate of the subjects with the TL1A isoform ratio above the cutoff as provided in the methods” is substituted by the phrase “the cutoff is such that the positive rate of the subjects with the TL1A isoform ratio above the cutoff as provided in the methods.” Additionally, for each embodiment described in this paragraph, also provided is a corresponding embodiment wherein the phrase “the positive rate of the subjects with the TL1A isoform ratio above the cutoff as provided in the methods” is substituted by the phrase “the cutoff is such that the positive rate of the subjects with the proxy marker above the cutoff as provided in the methods.”

As used herein, the term “positive rate,” when used in reference to a matter (e.g. in reference to subjects having TL1A isoform ratio above a cutoff or in reference to TL1A isoform ratio above a cutoff), means the percentage of the subjects or patients that are positive or determined to be positive for the matter (e.g. positive for having TL1A isoform ratio above a cutoff) over the whole subject or patient population. For another example, the positive rate of samples classified as having a TL1A isoform ratio above a cutoff means the percentage of samples classified as having a TL1A isoform ratio above a cutoff over the population of samples subjected to the classification. The term “negative rate,” when used in reference to a matter (e.g. in reference to subjects having TL1A isoform ratio above a cutoff or in reference to TL1A isoform ratio above a cutoff), means the percentage of the subjects or patients that are negative or determined to be negative for the matter (e.g. negative for having TL1A isoform ratio above a cutoff) over the whole subject or patient population. As a subject or a patient is either positive or negative for having a TL1A isoform ratio above a cutoff, the disclosure provides the corresponding embodiments for the negative rate of having TL1A isoform ratio above a cutoff, which negative rate is calculated as 100% minus the positive rate of having TL1A isoform ratio above the cutoff. Similarly, the disclosure provides the corresponding embodiments for the negative rate of having a proxy marker above a cutoff, which negative rate is calculated as 100% minus the positive rate of having proxy marker above the cutoff.

In various embodiments of the methods provided herein including in Sections 2, 5.1 (e.g. the preceding paragraphs), and 7, the TL1A isoform ratio or the proxy marker is predictive of a positive therapeutic response in the subject to a treatment with the inhibitor of TL1A activity or expression with an accuracy of at least 50%, The other embodiments described in this paragraph are provided for the various methods described herein including in Sections 2, 5.1 (e.g. the preceding paragraphs), and 7. In some embodiments, the TL1A isoform ratio or the proxy marker is predictive of a positive therapeutic response in the subject to a treatment with the inhibitor of TL1A activity or expression with an accuracy of at least 55%, In some embodiments, the TL1A isoform ratio or the proxy marker is predictive of a positive therapeutic response in the subject to a treatment with the inhibitor of TL1A activity or expression with an accuracy of at least 60%, In some embodiments, the TL1A isoform ratio or the proxy marker is predictive of a positive therapeutic response in the subject to a treatment with the inhibitor of TL1A activity or expression with an accuracy of at least 65%, In some embodiments, the TL1A isoform ratio or the proxy marker is predictive of a positive therapeutic response in the subject to a treatment with the inhibitor of TL1A activity or expression with an accuracy of at least 70%, In some embodiments, the TL1A isoform ratio or the proxy marker is predictive of a positive therapeutic response in the subject to a treatment with the inhibitor of TL1A activity or expression with an accuracy of at least 75%, In some embodiments, the TL1A isoform ratio or the proxy marker is predictive of a positive therapeutic response in the subject to a treatment with the inhibitor of TL1A activity or expression with an accuracy of at least 80%, In some embodiments, the TL1A isoform ratio or the proxy marker is predictive of a positive therapeutic response in the subject to a treatment with the inhibitor of TL1A activity or expression with an accuracy of at least 85%, In some embodiments, the TL1A isoform ratio or the proxy marker is predictive of a positive therapeutic response in the subject to a treatment with the inhibitor of TL1A activity or expression with an accuracy of at least 90%, In some embodiments, the TL1A isoform ratio or the proxy marker is predictive of a positive therapeutic response in the subject to a treatment with the inhibitor of TL1A activity or expression with an accuracy of at least 95%, In some embodiments, the TL1A isoform ratio or the proxy marker is predictive of a positive therapeutic response in the subject to a treatment with the inhibitor of TL1A activity or expression with an accuracy of about 50%, In some embodiments, the TL1A isoform ratio or the proxy marker is predictive of a positive therapeutic response in the subject to a treatment with the inhibitor of TL1A activity or expression with an accuracy of about 55%, In some embodiments, the TL1A isoform ratio or the proxy marker is predictive of a positive therapeutic response in the subject to a treatment with the inhibitor of TL1A activity or expression with an accuracy of about 60%, In some embodiments, the TL1A isoform ratio or the proxy marker is predictive of a positive therapeutic response in the subject to a treatment with the inhibitor of TL1A activity or expression with an accuracy of about 65%, In some embodiments, the TL1A isoform ratio or the proxy marker is predictive of a positive therapeutic response in the subject to a treatment with the inhibitor of TL1A activity or expression with an accuracy of about 70%, In some embodiments, the TL1A isoform ratio or the proxy marker is predictive of a positive therapeutic response in the subject to a treatment with the inhibitor of TL1A activity or expression with an accuracy of about 75%, In some embodiments, the TL1A isoform ratio or the proxy marker is predictive of a positive therapeutic response in the subject to a treatment with the inhibitor of TL1A activity or expression with an accuracy of about 80%, In some embodiments, the TL1A isoform ratio or the proxy marker is predictive of a positive therapeutic response in the subject to a treatment with the inhibitor of TL1A activity or expression with an accuracy of about 85%, In some embodiments, the TL1A isoform ratio or the proxy marker is predictive of a positive therapeutic response in the subject to a treatment with the inhibitor of TL1A activity or expression with an accuracy of about 90%, In some embodiments, the TL1A isoform ratio or the proxy marker is predictive of a positive therapeutic response in the subject to a treatment with the inhibitor of TL1A activity or expression with an accuracy of about 95%, In some embodiments, the TL1A isoform ratio or the proxy marker is predictive of a positive therapeutic response in the subject to a treatment with the inhibitor of TL1A activity or expression with an accuracy of from 60% to 95%, from 65% to 95%, from 70% to 95%, from 75% to 95%, from 80% to 95%, from 85% to 95%, from 90% to 95%, from 60% to 90%, from 65% to 90%, from 70% to 90%, from 75% to 90%, from 80% to 90%, from 85% to 90%, from 60% to 85%, from 65% to 85%, from 70% to 85%, from 75% to 85%, from 80% to 85%, from 60% to 80%, from 65% to 80%, from 70% to 80%, from 75% to 80%, from 60% to 75%, from 65% to 75%, from 70% to 75%, from 60% to 70%, from 65% to 70%, or from 60% to 65%, As is clear from the description, the element “the TL1A isoform ratio or the proxy marker is predictive of” means that each embodiment described in this paragraph further includes two embodiments with one reciting “the TL1A isoform ratio is predictive of” and the other reciting “the proxy marker is predictive of.” Furthermore, for each embodiment described in this paragraph, the element “the proxy marker is predictive of” is (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation. Additionally, for each embodiment described in this paragraph, an additional embodiment is provided wherein the element “the TL1A isoform ratio is predictive of” is “the TL1A isoform ratio above a cutoff is predictive of.”

Accordingly, in various embodiments of the methods provided herein including in Sections 2, 5.1 (e.g. the preceding paragraphs), and 7, the TL1A isoform ratio above the cutoff predicts a positive therapeutic response in the subject to a treatment with the inhibitor of TL1A activity or expression with an accuracy of at least 50%, The other embodiments described in this paragraph are provided for the various methods described herein including in Sections 2, 5.1 (e.g. the preceding paragraphs), and 7. In some embodiments, the TL1A isoform ratio above the cutoff predicts a positive therapeutic response in the subject to a treatment with the inhibitor of TL1A activity or expression with an accuracy of at least 55%, In some embodiments, the TL1A isoform ratio above the cutoff predicts a positive therapeutic response in the subject to a treatment with the inhibitor of TL1A activity or expression with an accuracy of at least 60%, In some embodiments, the TL1A isoform ratio above the cutoff predicts a positive therapeutic response in the subject to a treatment with the inhibitor of TL1A activity or expression with an accuracy of at least 65%, In some embodiments, the TL1A isoform ratio above the cutoff predicts a positive therapeutic response in the subject to a treatment with the inhibitor of TL1A activity or expression with an accuracy of at least 70%, In some embodiments, the TL1A isoform ratio above the cutoff predicts a positive therapeutic response in the subject to a treatment with the inhibitor of TL1A activity or expression with an accuracy of at least 75%, In some embodiments, the TL1A isoform ratio above the cutoff predicts a positive therapeutic response in the subject to a treatment with the inhibitor of TL1A activity or expression with an accuracy of at least 80%, In some embodiments, the TL1A isoform ratio above the cutoff predicts a positive therapeutic response in the subject to a treatment with the inhibitor of TL1A activity or expression with an accuracy of at least 85%, In some embodiments, the TL1A isoform ratio above the cutoff predicts a positive therapeutic response in the subject to a treatment with the inhibitor of TL1A activity or expression with an accuracy of at least 90%, In some embodiments, the TL1A isoform ratio above the cutoff predicts a positive therapeutic response in the subject to a treatment with the inhibitor of TL1A activity or expression with an accuracy of at least 95%, In some embodiments, the TL1A isoform ratio above the cutoff predicts a positive therapeutic response in the subject to a treatment with the inhibitor of TL1A activity or expression with an accuracy of about 50%, In some embodiments, the TL1A isoform ratio above the cutoff predicts a positive therapeutic response in the subject to a treatment with the inhibitor of TL1A activity or expression with an accuracy of about 55%, In some embodiments, the TL1A isoform ratio above the cutoff predicts a positive therapeutic response in the subject to a treatment with the inhibitor of TL1A activity or expression with an accuracy of about 60%, In some embodiments, the TL1A isoform ratio above the cutoff predicts a positive therapeutic response in the subject to a treatment with the inhibitor of TL1A activity or expression with an accuracy of about 65%, In some embodiments, the TL1A isoform ratio above the cutoff predicts a positive therapeutic response in the subject to a treatment with the inhibitor of TL1A activity or expression with an accuracy of about 70%, In some embodiments, the TL1A isoform ratio above the cutoff predicts a positive therapeutic response in the subject to a treatment with the inhibitor of TL1A activity or expression with an accuracy of about 75%, In some embodiments, the TL1A isoform ratio above the cutoff predicts a positive therapeutic response in the subject to a treatment with the inhibitor of TL1A activity or expression with an accuracy of about 80%, In some embodiments, the TL1A isoform ratio above the cutoff predicts a positive therapeutic response in the subject to a treatment with the inhibitor of TL1A activity or expression with an accuracy of about 85%, In some embodiments, the TL1A isoform ratio above the cutoff predicts a positive therapeutic response in the subject to a treatment with the inhibitor of TL1A activity or expression with an accuracy of about 90%, In some embodiments, the TL1A isoform ratio above the cutoff predicts a positive therapeutic response in the subject to a treatment with the inhibitor of TL1A activity or expression with an accuracy of about 95%, In some embodiments, the TL1A isoform ratio above the cutoff predicts a positive therapeutic response in the subject to a treatment with the inhibitor of TL1A activity or expression with an accuracy of from 60% to 95%, from 65% to 95%, from 70% to 95%, from 75% to 95%, from 80% to 95%, from 85% to 95%, from 90% to 95%, from 60% to 90%, from 65% to 90%, from 70% to 90%, from 75% to 90%, from 80% to 90%, from 85% to 90%, from 60% to 85%, from 65% to 85%, from 70% to 85%, from 75% to 85%, from 80% to 85%, from 60% to 80%, from 65% to 80%, from 70% to 80%, from 75% to 80%, from 60% to 75%, from 65% to 75%, from 70% to 75%, from 60% to 70%, from 65% to 70%, or from 60% to 65%, Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.”

The term “accuracy,” when used in reference to a test for selecting patients for treatment or in the context of a performing metrics of a test for selecting patients for treatment, means the summation of the performance for correctly calling the true positive and correctly calling the true negatives. Accuracy can be calculated as (true positive+true negative)/(true positive+true negative+false positive+false negative)). Accuracy can also be calculated as the balanced accuracy, which is the geometric mean of recall, or sensitivity, across all labels or treatment response phenotypes.

In various embodiments of the methods provided herein including in Sections 2, 5.1 (e.g. the preceding paragraphs), and 7, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 25%, The other embodiments described in this paragraph are provided for the various methods described herein including in Sections 2, 5.1 (e.g. the preceding paragraphs), and 7. In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 26%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 27%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 28%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 29%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 30%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 31%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 32%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 33%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 34%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 35%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 40%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 51%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of at least 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 25%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 26%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 27%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 28%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 29%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 30%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 31%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 32%, In some embodiments, the TL1A isoform ratio above the cutoff predicts an increase in TL1A protein expression in a sample obtained from a subject as compared to a reference level of TL1A protein expression (e.g., level in a tissue not affected by (BD) with a positive predictive value of about 33%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 34%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 35%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 40%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 51%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value of about 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a positive predictive value from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 25% to 70%, from 30% to 70%, from 35% to 70%, from 40% to 70%, from 45% to 70%, from 50% to 70%, from 55% to 70%, from 60% to 70%, from 65% to 70%, from 25% to 65%, from 30% to 65%, from 35% to 65%, from 40% to 65%, from 45% to 65%, from 50% to 65%, from 55% to 65%, from 60% to 65%, from 25% to 60%, from 30% to 60%, from 35% to 60%, from 40% to 60%, from 45% to 60%, from 50% to 60%, from 55% to 60%, from 25% to 55%, from 30% to 55%, from 35% to 55%, from 40% to 55%, from 45% to 55%, from 50% to 55%, from 25% to 50%, from 30% to 50%, from 35% to 50%, from 40% to 50%, from 45% to 50%, from 25% to 45%, from 30% to 45%, from 35% to 45%, from 40% to 45%, from 25% to 40%, from 30% to 40%, from 35% to 40%, from 25% to 35%, from 30% to 35%, or from 25% to 30%, In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.” Still further, for each embodiment described in this paragraph, also provided is a corresponding embodiment wherein the phrase “the TL1A isoform ratio above the cutoff predicts” is substituted by the phrase “the TL1A isoform ratio is predictive of.” Furthermore, for each embodiment described in this paragraph, also provided is a corresponding embodiment wherein the phrase “the TL1A isoform ratio above the cutoff predicts” is substituted by the phrase “the proxy marker is predictive of.” Additionally, for the embodiment described in this paragraph, the element “the proxy marker is predictive of” can be (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation.

In various embodiments of the methods provided herein including in Sections 2, 5.1 (e.g. the preceding paragraphs), and 7, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 40%, The other embodiments described in this paragraph are provided for the various methods described herein including in Sections 2, 5.1 (e.g. the preceding paragraphs), and 7. In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of at least 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value of about 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a negative predictive value from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%, In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.” Still further, for each embodiment described in this paragraph, also provided is a corresponding embodiment wherein the phrase “the TL1A isoform ratio above the cutoff predicts” is substituted by the phrase “the TL1A isoform ratio is predictive of.” Furthermore, for each embodiment described in this paragraph, also provided is a corresponding embodiment wherein the phrase “the TL1A isoform ratio above the cutoff predicts” is substituted by the phrase “the proxy marker is predictive of.” Additionally, for the embodiment described in this paragraph, the element “the proxy marker is predictive of” can be (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation.

In various embodiments of the methods provided herein including in Sections 2, 5.1 (e.g. the preceding paragraphs), and 7, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 20%, The other embodiments described in this paragraph are provided for the various methods described herein including in Sections 2, 5.1 (e.g. the preceding paragraphs), and 7. In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 25%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 30%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 35%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 40%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of at least 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 20%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 25%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 30%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 35%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 40%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity of about 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a sensitivity from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 20% to 85%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 20% to 65%, from 25% to 65%, from 30% to 65%, from 35% to 65%, from 40% to 65%, from 45% to 65%, from 50% to 65%, from 55% to 65%, from 60% to 65%, from 20% to 60%, from 25% to 60%, from 30% to 60%, from 35% to 60%, from 40% to 60%, from 45% to 60%, from 50% to 60%, from 55% to 60%, from 20% to 55%, from 25% to 55%, from 30% to 55%, from 35% to 55%, from 40% to 55%, from 45% to 55%, from 50% to 55%, from 20% to 50%, from 25% to 50%, from 30% to 50%, from 35% to 50%, from 40% to 50%, from 45% to 50%, from 20% to 45%, from 25% to 45%, from 30% to 45%, from 35% to 45%, from 40% to 45%, from 20% to 40%, from 25% to 40%, from 30% to 40%, from 35% to 40%, from 20% to 35%, from 25% to 35%, from 30% to 35%, from 20% to 30%, from 25% to 30%, or from 20% to 25%, In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.” Still further, for each embodiment described in this paragraph, also provided is a corresponding embodiment wherein the phrase “the TL1A isoform ratio above the cutoff predicts” is substituted by the phrase “the TL1A isoform ratio is predictive of.” Furthermore, for each embodiment described in this paragraph, also provided is a corresponding embodiment wherein the phrase “the TL1A isoform ratio above the cutoff predicts” is substituted by the phrase “the proxy marker is predictive of.” Additionally, for the embodiment described in this paragraph, the element “the proxy marker is predictive of” can be (1) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation.

In various embodiments of the methods provided herein including in Sections 2, 5.1 (e.g. the preceding paragraphs), and 7, the TL1A isoform ratio above the cutoff predicts an increase in TL1A protein expression in a sample obtained from a subject as compared to a reference level of TL1A protein expression (e.g., level in a tissue not affected by IBD) with a specificity of at least 40%, The other embodiments described in this paragraph are provided for the various methods described herein including in Sections 2, 5.1 (e.g. the preceding paragraphs), and 7. In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of at least 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 40%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 45%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity of about 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with a specificity from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%, In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.” Still further, for each embodiment described in this paragraph, also provided is a corresponding embodiment wherein the phrase “the TL1A isoform ratio above the cutoff predicts” is substituted by the phrase “the TL1A isoform ratio is predictive of.” Furthermore, for each embodiment described in this paragraph, also provided is a corresponding embodiment wherein the phrase “the TL1A isoform ratio above the cutoff predicts” is substituted by the phrase “the proxy marker is predictive of.” Additionally, for the embodiment described in this paragraph, the element “the proxy marker is predictive of” can be (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation.

In various embodiments of the methods provided herein including in Sections 2, 5.1 (e.g. the preceding paragraphs), and 7, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 50%, The other embodiments described in this paragraph are provided for the various methods described herein including in Sections 2, 5.1 (e.g. the preceding paragraphs), and 7. In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of at least 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 50%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 55%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 60%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 65%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 70%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 75%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 80%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 85%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 90%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of about 95%, In some embodiments, the TL1A isoform ratio above the cutoff predicts an increase in TL1A protein expression in a sample obtained from a subject as compared to a reference level of TL1A protein expression (e.g., level in a tissue not affected by IBD) with an accuracy of from 60% to 95%, from 65% to 95%, from 70% to 95%, from 75% to 95%, from 80% to 95%, from 85% to 95%, from 90% to 95%, from 60% to 90%, from 65% to 90%, from 70% to 90%, from 75% to 90%, from 80% to 90%, from 85% to 90%, from 60% to 85%, from 65% to 85%, from 70% to 85%, from 75% to 85%, from 80% to 85%, from 60% to 80%, from 65% to 80%, from 70% to 80%, from 75% to 80%, from 60% to 75%, from 65% to 75%, from 70% to 75%, from 60% to 70%, from 65% to 70%, or from 60% to 65%, In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the TL1A isoform ratio above the cutoff predicts” is substituted by the phrase “the cutoff is such that the TL1A isoform ratio above the cutoff is predictive of.” Additionally, for each embodiment described in this paragraph, also provided is a corresponding embodiment wherein the phrase “the TL1A isoform ratio above the cutoff predicts” is substituted by the phrase “the cutoff is such that the proxy marker the above the cutoff is predictive of.” Still further, for each embodiment described in this paragraph, also provided is a corresponding embodiment wherein the phrase “the TL1A isoform ratio above the cutoff predicts” is substituted by the phrase “the TL1A isoform ratio is predictive of.” Furthermore, for each embodiment described in this paragraph, also provided is a corresponding embodiment wherein the phrase “the TL1A isoform ratio above the cutoff predicts” is substituted by the phrase “the proxy marker is predictive of.” Additionally, for the embodiment described in this paragraph, the element “the proxy marker is predictive of” can be (i) “the proxy marker above a cutoff is predictive of” if the proxy marker and the TL1A isoform ratio has a positive correlation, or (ii) “the proxy marker below a cutoff is predictive of” if the proxy marker and the TL1A isoform ratio has a negative correlation.

The disclosure provides that for the embodiments of the preceding 19 paragraphs, when TL1A isoform ratio above a cutoff achieves a certain metrics such as PPV, specificity, sensitivity, NPV, positive rate, or accuracy, a cutoff is determined and provided such that the TL1A above the cutoff can achieve the metrics recited in the preceding 19 paragraphs. Accordingly, In some embodiments of the methods provided herein including Sections 2, 5.1 (e.g. the preceding paragraphs such as the preceding 19 paragraphs), and 7, the cutoff is such that the TL1A isoform ratio above the cutoff achieve any of the PPV, specificity, sensitivity, NPV, positive rate, or accuracy described in the preceding paragraphs such as the preceding 19 paragraphs. Specifically, in some embodiments of the methods provided herein, including in Sections 2, 5.1 (e.g. the preceding paragraphs such as the preceding 19 paragraphs), and 7, the cutoff is such that the TL1A isoform ratio above the cutoff is predictive of a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, or 95%, In some embodiments of the methods provided herein, including in Sections 2, 5.1 (e.g. the preceding paragraphs such as the preceding 19 paragraphs), and 7, the cutoff is such that the TL1A isoform ratio above the cutoff is predictive of a positive therapeutic response in the subject to a treatment with the inhibitor of TL1A activity or expression with a specificity of at least about 50%, 55%, 60%, 65%, 70%, 75%, 80%, 85%, 90%, or 95%, In some embodiments of the methods provided herein, including in Sections 2, 5.1 (e.g. the preceding paragraphs such as the preceding 19 paragraphs), and 7, the cutoff is such that the TL1A isoform ratio above the cutoff is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%, In some embodiments of the methods provided herein, including in Sections 2, 5.1 (e.g. the preceding paragraphs such as the preceding 19 paragraphs), and 7, the cutoff is such that the TL1A isoform ratio above the cutoff is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%, In some embodiments of the methods provided herein, including in Sections 2, 5.1 (e.g. the preceding paragraphs such as the preceding 19 paragraphs), and 7, the cutoff is such that the TL1A isoform ratio above the cutoff is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%, In some embodiments of the methods provided herein, including in Sections 2, 5.1 (e.g. the preceding paragraphs such as the preceding 19 paragraphs), and 7, the cutoff is such that the TL1A isoform ratio above the cutoff is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%, In some embodiments of the methods provided herein, including in this paragraph, the cutoff can be determined as described in Sections 5.1.

Alternatively, the disclosure provides that for the embodiments of the preceding 20 paragraphs, when the proxy marker above a cutoff (if the proxy marker has positive correlation with the TL1A isoform ratio or below a cutoff if the proxy marker has a negative correlation with the TL1A isoform ratio) achieves a certain metrics such as PPV, specificity, sensitivity, NPV, positive rate, or accuracy, a cutoff is determined and provided such that the TL1A above the cutoff can achieve the metrics recited in the preceding 20 paragraphs. Accordingly, in some embodiments of the methods provided herein including Sections 2, 5.1 (e.g. the preceding paragraphs such as the preceding 20 paragraphs), and 7, the cutoff is such that the TL1A isoform ratio above the cutoff (if the proxy marker has positive correlation with the TL1A isoform ratio or below a cutoff if the proxy marker has a negative correlation with the TL1A isoform ratio) achieves any of the PPV, specificity, sensitivity, NPV, positive rate, or accuracy described in the preceding paragraphs such as the preceding 20 paragraphs. In some embodiments of the methods provided herein, including in Sections 2, 5.1 (e.g. the preceding paragraphs such as the preceding 20 paragraphs), and 7, the cutoff is such that the proxy marker above the cutoff (if the proxy marker has positive correlation with the TL1A isoform ratio or below a cutoff if the proxy marker has a negative correlation with the TL1A isoform ratio) is predictive of a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, or 95%, In some embodiments of the methods provided herein, including in Sections 2, 5.1 (e.g. the preceding paragraphs such as the preceding 20 paragraphs), and 7, the cutoff is such that the proxy marker above the cutoff (if the proxy marker has positive correlation with the TL1A isoform ratio or below a cutoff if the proxy marker has a negative correlation with the TL1A isoform ratio) is predictive of a positive therapeutic response in the subject to a treatment with the inhibitor of TL1A activity or expression with a specificity of at least about 50%, 55%, 60%, 65%, 70%, 75%, 80%, 85%, 90%, or 95%, In some embodiments of the methods provided herein, including in Sections 2, 5.1 (e.g. the preceding paragraphs such as the preceding 20 paragraphs), and 7, the cutoff is such that the proxy marker above the cutoff (if the proxy marker has positive correlation with the TL1A isoform ratio or below a cutoff if the proxy marker has a negative correlation with the TL1A isoform ratio) is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%, In some embodiments of the methods provided herein, including in Sections 2, 5.1 (e.g. the preceding paragraphs such as the preceding 20 paragraphs), and 7, the cutoff is such that the proxy marker above the cutoff (if the proxy marker has positive correlation with the TL1A isoform ratio or below a cutoff if the proxy marker has a negative correlation with the TL1A isoform ratio) is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%, In some embodiments of the methods provided herein, including in Sections 2, 5.1 (e.g. the preceding paragraphs such as the preceding 20 paragraphs), and 7, the cutoff is such that the proxy marker above the cutoff (if the proxy marker has positive correlation with the TL1A isoform ratio or below a cutoff if the proxy marker has a negative correlation with the TL1A isoform ratio) is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%, In some embodiments of the methods provided herein, including in Sections 2, 5.1 (e.g. the preceding paragraphs such as the preceding 20 paragraphs), and 7, the cutoff is such that the proxy marker above the cutoff (if the proxy marker has positive correlation with the TL1A isoform ratio or below a cutoff if the proxy marker has a negative correlation with the TL1A isoform ratio) is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%, In some embodiments of the methods provided herein, including in this paragraph, the cutoff can be determined as described in Sections 5.1.

As used herein, the term “positive therapeutic response” is intended to mean improvement of one or more aspects of the symptoms of the inflammatory, fibrotic, or fibrostenotic disease or condition in a subject after treatment with the inhibitor of TL1A activity or expression provided herein, as determined by clinical parameters for such disease or condition. As is clear from the description herein, the inflammatory, fibrotic, or fibrostenotic disease or condition can be inflammatory bowel disease such as Crohn's disease or ulcerative colitis. In the context of ulcerative colitis, such improvement of the disease symptoms can be measured by Mayo Score (based on stool frequency, rectal bleeding, the endoscopic appearance of the mucosa (endoscopic score), and a Physician's Global Assessment (PGA), each of which were scored on a scale from 0 to 3, giving a Mayo Score of 0-12) or Modified Mayo Score (based on stool frequency, rectal bleeding, and the endoscopic appearance of the mucosa (endoscopic score), each of which were scored on a scale from 0 to 3, giving a Modified Mayo Score of 0-9), as known and practiced in the field. For example, in one embodiment, the positive therapeutic response in UC can be an improvement of the 3-component Modified Mayo Score, as determined by a reduction from baseline by ≥2 points and ≥30% in the 3-component Modified Mayo Score, accompanied by a reduction ≥1 in rectal bleeding subscore or absolute rectal bleeding subscore ≤1. In the context of Crohn's disease, such improvement of the disease symptoms can be measured by Crohn's disease activity index (CDAI), simple endoscopy score for Crohn's disease (SES-CD, e.g. as further described in Gastrointest Endosc 2004; 60:505-512), or both CDAI and SES-CD, as known and practiced in the field. For example, in one embodiment, the positive therapeutic response can be an improvement of the CDAI, as determined by a reduction from baseline by ≥100 in CDAI. In another example, the positive therapeutic response can be a clinical remission, as defined below. In another example, the positive therapeutic response can be an endoscopic improvement, as defined below.

As used herein, the term “clinical remission” is intended to mean (i) the symptoms of the inflammatory, fibrotic, or fibrostenotic disease or condition in a subject have improved to the extent that the symptoms are absent, close to be absent, or moving close towards disappearance, or (ii) the inflammatory, fibrotic, or fibrostenotic disease or condition in the subject becomes a mild or less than a mild disease, after treatment with the inhibitor of TL1A activity or expression provided herein, as determined by clinical parameters for such disease or condition. As is clear from the description herein, the inflammatory, fibrotic, or fibrostenotic disease or condition can be inflammatory bowel disease such as Crohn's disease or ulcerative colitis. In the context of ulcerative colitis, the clinical remission can be measured by Mayo Score or Modified Mayo Score, each as described above in the definition of “positive therapeutic response”. For example, in one embodiment, the clinical remission in UC can be determined based on the 3-component Modified Mayo Score as an endoscopic subscore of 0 or 1, rectal bleeding subscore of 0, and stool frequency subscore of 0 or 1 and not greater than baseline. In the context of Crohn's disease, such improvement of the disease symptoms can be measured by CDAI, SES-CD, or both CDAI and SES-CD, as known and practiced in the field. For example, in one embodiment, the clinical remission in CD can be determined based on the CDAI, as CDAI<150.

As used herein, the term “endoscopic improvement” is intended to mean (i) the inflammation in the mucosa of the digestive tract of the subject is absent, close to be absent, or moving close towards disappearance, or (ii) substantial reduction (≥50% reduction) of the inflammation in the mucosa of the digestive tract in a subject, after treatment with the inhibitor of TL1A activity or expression provided herein, as determined by a colonoscopy, a sigmoidoscopy, or other applicable endoscopy. As is clear from the description herein, the subject can be a subject affected by inflammatory, fibrotic, or fibrostenotic disease or condition, for example, inflammatory bowel disease such as Crohn's disease or ulcerative colitis. In the context of ulcerative colitis, the endoscopic improvement can be measured by the endoscopy subscore that is used as a component of the Mayo Score or Modified Mayo Score. For example, in one embodiment, the endoscopic improvement in UC can be determined based on endoscopy subscore≤1 with no friability. In the context of Crohn's disease, such endoscopic improvement can be measured by SES-CD, as known and practiced in the field. For example, in one embodiment, the endoscopic improvement in CD can be determined based on decrease in simple endoscopy score for Crohn's disease (SES-CD)≥50% from baseline.

In some embodiments of the methods provided herein including in Sections 2, 5.1 (e.g. the preceding paragraphs), and 7, the positive therapeutic response is clinical remission. In some embodiments of the methods provided herein including in Sections 2, 5.1 (e.g. the preceding paragraphs), and 7, the positive therapeutic response is endoscopic improvement. In some embodiments of the methods provided herein including in Sections 2, 5.1 (e.g. the preceding paragraphs), and 7, the positive therapeutic response is histologic remission (determined by Geboes score≤3.1, as known and practiced in the field of IBD). In some embodiments of the methods provided herein including in Sections 2, 5.1 (e.g. the preceding paragraphs), and 7, the positive therapeutic response is histologic-endoscopic mucosal improvement (determined by Geboes score≤3.1 and endoscopy subscore≤1 with no friability). In some embodiments of the methods provided herein including in Sections 2, 5.1 (e.g. the preceding paragraphs), and 7, the positive therapeutic response is histologic improvement (determined by Geboes score≤3.1). In some embodiments of the methods provided herein including in Sections 2, 5.1 (e.g. the preceding paragraphs), and 7, the positive therapeutic response is mucosal healing (determined by Geboes score≤2B.1 and endoscopy subscore of ≤1). In some embodiments of the methods provided herein including in Sections 2, 5.1 (e.g. the preceding paragraphs), and 7, the positive therapeutic response is inflammatory bowel disease questionnaire (IBDQ) response, as determined by ≥16-point increase from baseline.

In some embodiments of the methods provided herein including in Sections 2, 5.1 (e.g. the preceding paragraphs), and 7, the positive therapeutic response is determined at the end of induction dose regimen. In some embodiments of the methods provided herein including in Sections 2, 5.1 (e.g. the preceding paragraphs), and 7, the positive therapeutic response is determined at week 12.

The disclosure specifically provides that the various embodiments of positive predictive value as provided herein (including in Sections 2, 5.1 (e.g. the preceding paragraphs), and 7) can be combined with the various embodiments of specificity as provided herein (Sections 2, 5.1 (e.g. the preceding paragraphs), and 7) in various embodiments of the methods, kit, and compositions as provided herein (Sections 2, 5.1 (e.g. the preceding paragraphs), and 7).

The disclosure also specifically provides that the various embodiments of positive predictive value as provided herein (including in Sections 2, 5.1 (e.g. the preceding paragraphs), and 7) can be combined with the various embodiments of the positive rate of the TL1A isoform ratio above the cutoff as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7). The disclosure additionally specifically provides that the various embodiments of positive predictive value as provided herein (including in Sections 2, 5.1 (e.g. the preceding paragraphs), and 7) can be combined with the various embodiments of the positive rate of the proxy marker above the cutoff (if the proxy marker has a positive correlation with TL1A isoform ratio) as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7). The disclosure further specifically provides that the various embodiments of positive predictive value as provided herein (including in Sections 2, 5.1 (e.g. the preceding paragraphs), and 7) can be combined with the various embodiments of the positive rate of the proxy marker below the cutoff (if the proxy marker has a negative correlation with TL1A isoform ratio) as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7).

The disclosure further specifically provides that the various embodiments of positive predictive value as provided herein (including in Sections 2, 5.1 (e.g. the preceding paragraphs), and 7) can be combined with the various embodiments of sensitivity as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7).

The disclosure additionally provides that the various embodiments of positive predictive value as provided herein (including in Sections 2, 5,1 (e.g. the preceding paragraphs), and 7) can be combined with the various embodiments of negative predictive value as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7).

The disclosure also provides that the various embodiments of negative predictive value as provided herein (including in Sections 2, 5.1 (e.g. the preceding paragraphs), and 7) can be combined with the various embodiments of specificity as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), $0.2, 5.4, 5.5, 5.6, and 7).

The disclosure specifically provides that the various embodiments of negative predictive value as provided herein (including in Sections 2, 5.1 (e.g. the preceding paragraphs), and 7) can be combined with the various embodiments of sensitivity as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7).

The disclosure further specifically provides that the various embodiments of sensitivity as provided herein (including in Sections 2, 5.1 (e.g. the preceding paragraphs), and 7) can be combined with the various embodiments of specificity as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7).

The disclosure also specifically provides that the various embodiments of sensitivity as provided herein (including in Sections 2, 5.1 (e.g. the preceding paragraphs), and 7) can be combined with the various embodiments of the positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio) as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7).

The disclosure additionally provides that the various embodiments of specificity as provided herein (including in Sections 2, 5.1 (e.g. the preceding paragraphs), and 7) can be combined with the various embodiments of the positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio) as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5.4, 5.5, 5.6, and 7).

The disclosure specifically provides that the various embodiments of negative predictive value as provided herein (including in Sections 2, 5.1 (e.g. the preceding paragraphs), and 7) can be combined with the various embodiments of the positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio) as provided herein (including in Sections 2, 5.1 (e.g. the preceding paragraphs), and 7) in various embodiments of the methods, kit, and compositions as provided herein (including in Sections 2, 5.1 (e.g. the preceding paragraphs), 5.2, 5,4, 5.5, 5.6, and 7).

Additionally, the disclosure provides that positive predictive value, negative predictive value, sensitivity, specificity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7).

In some embodiments, the disclosure provides that negative predictive value, sensitivity, specificity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that positive predictive value, sensitivity, specificity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5,6, and 7). In some embodiments, the disclosure provides that positive predictive value, negative predictive value, specificity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that positive predictive value, negative predictive value, sensitivity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that positive predictive value, negative predictive value, sensitivity, and/or specificity, each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7).

In some embodiments, the disclosure provides that positive predictive value, negative predictive value, and/or sensitivity, each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs). 5.2, 5.4, 5.5, 5.6, and 7). In some embodiments, the disclosure provides that positive predictive value, negative predictive value, and/or specificity, each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5,4, 5.5, 5.6, and 7). In some embodiments, the disclosure provides that positive predictive value, negative predictive value, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that positive predictive value, sensitivity, and/or specificity, each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that positive predictive value, sensitivity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that positive predictive value, specificity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5,6, and 7). In some embodiments, the disclosure provides that negative predictive value, sensitivity, and/or specificity, each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that negative predictive value, sensitivity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5.6, and 7). In some embodiments, the disclosure provides that negative predictive value, specificity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5,6, and 7). In some embodiments, the disclosure provides that sensitivity, specificity, and/or positive rate of TL1A isoform ratio above a cutoff or proxy marker (above a cutoff if the proxy marker has positive correlation with the TL1A isoform ratio or below a cutoff if the proxy marker has a negative correlation with the TL1A isoform ratio), each as provided herein (including in Sections 2, 5.1 (e.g. the preceding paragraphs), and 7), can be combined in various combinations in various embodiments of the methods, kit, and compositions as provided herein (including in Sections 2, 5.1 (e.g. the preceding paragraphs), 5.2, 5.4, 5.5, 5,6, and 7).

For various embodiments of the methods provided herein, including in Sections 2, 5.1 (including in this paragraph), and 7, the method comprises various elements or combinations of elements provided herein, for example, as described in the following: further embodiments of the therapeutic agent including anti-TL1A therapeutic agents and anti-TL1A antibodies (e.g., anti-TL1A embodiments with exemplary CDRs, framework sequences, constant region sequences. Fc mutations, variable regions, Fc regions, and other properties) are provided in Sections 2, 5.3, and 7; assays for detecting the TL1A isoforms or proxy markers are provided in Sections 2, 5.2, 5.6, and 7; assays for validating the TL1A isoforms or proxy markers for the methods are provided in in Sections 2, 5.2, 5.6, and 7; methods of preparing the samples for detecting the TL1A isoforms or proxy markers are provided in Section 5.7; TL1A isoforms, TL1A isoform ratios, proxy markers, or other criteria used for selecting patients or subjects for the methods are provided in Section 2, 5.1 (including in this paragraph), and 7; methods for selecting the patients for the treatment is provided in Section 2, 5.1 (including in this paragraph), and 7; pharmaceutical compositions for the anti-TL1A antibodies are described and provided in Sections 2, 5.3, 5.4, and 7; the step of, the software for, and the system for interpreting the TL1A isoforms, TL1A isoform ratios, or proxy markers are described in Section 2, 5.1 (including in this paragraph), 5.6, and 7; and/or further specific and validated embodiments for the therapeutic agents against TL1A and the methods of using the same are provided in Sections 2, 5.3 (e.g. Section 5.3.1.5), and 7. As such, the disclosure provides the various combinations of the therapeutic agent against TL1A, the pharmaceutical compositions of such therapeutic agent against TL1A, the methods of generating the therapeutic agent against TL1A, the methods of assaying the TL1A isoforms or proxy markers, the methods of preparing the sample, the methods of interpreting the TL1A isoforms, TL1A isoform ratio, or proxy markers, and/or the methods of using the therapeutic agent against TL1A for treatment.

As is clear from the above description, the patients or subjects are selected with the methods of selecting patients or subjects as provided herein including in Sections 2, 5.1 (including in the preceding paragraphs), and 7, and then subject to the various methods of treatment as provided herein including in Sections 2, 5.1 (including in the preceding paragraphs), and 7.

In some embodiments of the methods provided herein, including in Sections 2, 5.1 (including in the preceding paragraphs), and 7, the TL1A isoform ratio is TL1A isoform ratio in a diseased tissue afflicted by the inflammatory, fibrotic, or fibrostenotic disease or condition. In certain embodiments of the methods provided herein, including in Sections 2, 5.1 (including in the preceding paragraphs), and 7, the diseased tissue is selected from the group consisting of ileum, colon, rectum, or small intestine. In one embodiment, the diseased tissue comprises or consists of ileum. In another embodiment, the diseased tissue comprises or consists of colon. In a further embodiment, the diseased tissue comprises or consists of rectum. In yet another embodiment, the diseased tissue comprises or consists of small intestine.

In certain embodiments of the methods provided herein, including in Sections 2, 5.1 (including in the preceding paragraphs), and 7, the TL1A isoform ratio is TL1A isoform ratio in a cell contributory to the inflammatory, fibrotic, or fibrostenotic disease or condition. In some embodiments of the methods provided herein, including in Sections 2, 5.1 (including in the preceding paragraphs), and 7, the cell contributory to the inflammatory, fibrotic, or fibrostenotic disease or condition is selected from the group consisting of a Goblet cell, a pericyte, a smooth muscle cell, an enterocyte, a clonocyte, a monocyte-derived dendritic cells (moDC), a resident macrophage, or a colon transit-amplifying (TA) cell. In one embodiment, the cell contributory to the inflammatory, fibrotic, or fibrostenotic disease or condition comprises or consists of a Goblet cell. In another embodiment, the cell contributory to the inflammatory, fibrotic, or fibrostenotic disease or condition comprises or consists of a pericyte. In a further embodiment, the cell contributory to the inflammatory, fibrotic, or fibrostenotic disease or condition comprises or consists of a smooth muscle cell. In yet another embodiment, the cell contributory to the inflammatory, fibrotic, or fibrostenotic disease or condition comprises or consists of an enterocyte. In one embodiment, the cell contributory to the inflammatory, fibrotic, or fibrostenotic disease or condition comprises or consists of a clonocyte. In another embodiment, the cell contributory to the inflammatory, fibrotic, or fibrostenotic disease or condition comprises or consists of a monocyte-derived dendritic cells (moDC). In a further embodiment, the cell contributory to the inflammatory, fibrotic, or fibrostenotic disease or condition comprises or consists of a resident macrophage. In yet another embodiment, the cell contributory to the inflammatory, fibrotic, or fibrostenotic disease or condition comprises or consists of a colon transit-amplifying (TA) cell.

In some embodiments of the methods provided herein, including in Sections 2, 5.1 (including in the preceding paragraphs), and 7, the TL1A isoform ratio comprises or consists of TL1A isoform ratio in blood of the subject. In one embodiment, the TL1A isoform ratio comprises or consists of the TL1A isoform ratio in bodily fluid of the subject.

For various embodiments of the methods provided herein, including in Sections 2, 5.1 (including in the preceding paragraphs), and 7, the inflammatory disease comprises or is inflammatory bowel disease.

For various embodiments of the methods provided herein, including in Sections 2, 5.1 (including in the preceding paragraphs), and 7, the inflammatory disease comprises or is Crohn's disease. In some embodiments, the IBD comprises or is Crohn's disease.

For various embodiments of the methods provided herein, including in Sections 2, 5.1 (including in the preceding paragraphs), and 7, the Crohn's disease comprises or consists of ileal, ileocolonic, or colonic Crohn's disease.

For various embodiments of the methods provided herein, including in Sections 2, 5.1 (including in the preceding paragraphs), and 7, the inflammatory disease comprises or is ulcerative colitis (UC). In some embodiments, the IBD comprises or is UC.

For various embodiments of the methods provided herein, including in Sections 2, 5.1 (including in the preceding paragraphs), and 7, the UC is medically refractory UC.

For various embodiments of the methods provided herein, including in Sections 2, 5.1 (including in the preceding paragraphs), and 7, the sample is a biological sample.

For various embodiments of the methods, kit, systems, processes, or compositions provided herein, including in Sections 2, 5 (including in the paragraphs of Section 5.1), and 7, the corresponding alternative methods, kit, systems, processes, or compositions are also provided herein in which “comprising” is replaced by “consisting of.” In various embodiments of the methods, kit, systems, processes, or compositions provided herein, including in Sections 2, 5 (including in the paragraphs of Section 5.1), and 7, the corresponding alternative methods, kit, systems, processes, or compositions are also provided herein in which “comprise(s)” is replaced by “consist(s) of.”

Additionally, the disclosure provides various assays for determining or detecting TL1A isoforms, TL1A isoform ratio, or proxy markers. As such, in various embodiments of the methods provided herein including in Sections 2, 5 (including in the paragraphs of Section 5.1), and 7, determining or detecting TL1A isoforms, TL1A isoform ratio, or proxy markers comprises or consists of assaying for TL1A isoforms or proxy markers via any assays as described in Sections 2, 5.2, 5.7, and 7. In some embodiments of the methods provided herein including in Sections 2, 5 (including in the paragraphs of Section 5.1), and 7, determining or detecting TL1A isoforms, TL1A isoform ratio, or proxy markers comprises or consists of determining or detecting TL1A isoforms or proxy markers via any assays as described in Sections 2, 5.2, 5.7, and 7. In some embodiments of the methods provided herein including in Sections 2, 5 (including in the paragraphs of Section 5.1), and 7, determining or detecting TL1A isoforms. TL1A isoform ratio, or proxy markers comprises or consists of determining or detecting the TL1A isoforms, TL1A isoform ratio, or proxy markers via any or any combination of method steps as described in Sections 2, 5.2, 5.7, and 7. Alternatively, in various embodiments of the methods provided herein including in Sections 2, 5 (including in the paragraphs of Section 5.1), and 7, the method further comprises assaying for TL1A isoforms, TL1A isoform ratio, or proxy markers via any assays as described in Sections 2, 5.2, 5.7, and 7. In certain embodiments of the methods provided herein including in Sections 2, 5 (including in the paragraphs of Section 5.1), and 7, the method further comprises assaying for TL1A isoforms, TL1A isoform ratio, or proxy markers via any or any combination of method steps as described in Sections 2, 5.2, 5.7, and 7.

Specifically, for various embodiments of the methods provided herein, including in Sections 2, 5 (including in the paragraphs of Section 5.1), and 7, the anti-TL1A antibody or antigen binding fragment thereof used in the methods is any one selected from the anti-TL1A antibodies and antigen binding fragments described in Section 5.3. Additionally, for various embodiments of the methods provided herein, including in Sections 2, 5 (including in the paragraphs of Section 5.1), and 7, the inhibitor of TL1A activity or expression is any one selected from the inhibitors of TL1A activity or expression described in Section 5.3. In one specific embodiment of the methods provided herein, including in Sections 2, 5 (including in the paragraphs of Section 5.1), and 7, the anti-TL1A antibody or antigen binding fragment thereof used in the method comprises or consists of any antibody or antigen binding fragment selected from those provided in Section 5.3. In another specific embodiment of the methods provided herein, including in Sections 2, 5 (including in the paragraphs of Section 5.1), and 7, the anti-TL1A antibody or antigen binding fragment thereof used in the method is selected from those listed in Table 15 and Table 20. In one specific embodiment of the methods provided herein, including in Sections 2, 5 (including in the paragraphs of Section 5.1), and 7, the subject in the method has the TL1A isoform or proxy marker described in Section 5.1. In one specific embodiment of the methods provided herein, including in Sections 2, 5 (including in the paragraphs of Section 5.1), and 7, the subject in the method has the TL1A isoform or proxy marker described in Section 5.1 and the anti-TL1A antibody or antigen binding fragment thereof used in the methods is any one selected from the anti-TL1A antibodies and antigen binding fragments described in Section 5.3.

In one embodiment of the methods provided herein, including in Sections 2, 5 (including in the paragraphs of Section 5.1), and 7, the TL1A isoform ratio above a cutoff is predictive of (i) an increase in a level of TNFSF15 (TL1A) protein expression in a sample obtained from a subject or patient, as compared to a reference level of TNFSF15 (TL1A) protein expression (e.g., derived from a normal individual), and/or (ii) an increase of a positive therapeutic response in IBD patients to a treatment with the TL1A inhibitor as compared to the reference level of response in patients not selected by the TL1A isoform ratio. In one embodiment of the methods provided herein, including in Sections 2, 5 (including in the paragraphs of Section 5.1), and 7, the proxy marker above a cutoff (if the proxy marker has a positive correlation with the TL1A isoform ratio or proxy marker below a cutoff if the proxy marker has a negative correlation with the TL1A isoform ratio) is predictive of (i) an increase in a level of TNFSF15 (TL1A) protein expression in a sample obtained from a subject or patient, as compared to a reference level of TNFSF15 (TL1A) protein expression (e.g., derived from a normal individual), and/or (ii) an increase of a positive therapeutic response in IBD patients to a treatment with the TL1A inhibitor as compared to the reference level of response in patients not selected by the TL1A isoform or proxy marker. In some embodiments of the methods provided herein (e.g. in this paragraph), the step of determining the TL1A isoform ratio or the proxy marker is performed using a system selected from the systems provided in Section 5.6. In some embodiments of the methods provided herein (e.g. in this paragraph), the step of determining the TL1A isoform ratio or the proxy marker is performed using a computer implemented method selected from the computer implemented methods provided in Section 5.6. In some embodiments of the methods provided herein (e.g. in this paragraph), the step of determining the TL1A isoform ratio or the proxy marker is performed as described in Sections 5.6 and 7.

A few methods were adopted and compared to determine the cutoff value for the TL1A isoform ratio or proxy marker that was used for selecting patients responsive to a treatment with an inhibitor of TL1A expression or activity (e.g. TL1A CDx positive or TL1A CDx negative). Two of these methods give equal weight to sensitivity and specificity with no ethical, cost and prevalence constraints. The first method uses the square of distance between the point (0, 1) on the upper left hand corner of ROC space and any point on ROC curve, i.e. d²=(1-sensitivity)²+(1-specificity)². In order to obtain the optimal cut off points, the square of this distance is minimized. In other words, this distance for each point on the ROC curve from the point (0, 1) can be calculated to find the optimal cut-off value. The second method called Youden index uses the maximum of vertical distance of ROC curve from the point (x, y) on diagonal line (chance line). Youden index maximizes the difference between sensitivity and (1-sepicificity): Youden Index=Sensitivity+Specificity−1. Thus, by maximizing Sensitivity+Specificity across various points on the ROC curve, the optimal cut-off point is calculated. The third method incorporates the financial costs for correct and false diagnosis and the costs of further work up for diagnosis. In fact, the consequence of each possible test outcome is ascertained to their costs and combining ROC analysis with utility-based decision theory can be used to determine the optimal cutoff. For example, given a disease with low prevalence and high cost of false positive diagnosis, the cutoff may be chosen at higher value to maximize specificity while for a disease occurring at high prevalence and missing diagnosis has a serious fatal consequence, a lower cutoff value would be selected to maximize sensitivity. In a specific example, the cutoff value is set for a sensitivity of 100%, 95%, 90%, 85%, 80%, 75%, 70%, 65%, 60%, 55%, 50%, 45%, 40%, 35%, 30%, 25%, or 20%, In another example, the cutoff value can be set for a pre-determined positive predictive value, e.g. 95%, 90%, 85%, 80%, 75%, 70%, 65%, 60%, 55%, 51%, 50%, 45%, 40%, 35%, 34%, 33%, 32%, 31%, 30%, 29%, 28%, 27%, 26%, or 25%, In other examples, the cutoff value can be set for a pre-determined positive rate, e.g. 30-40%, In one example, the cutoff value is set for a positive rate of about 25%, In one example, the cutoff value is set for a positive rate of about 26%, In one example, the cutoff value is set for a positive rate of about 27%, In one example, the cutoff value is set for a positive rate of about 28%, In one example, the cutoff value is set for a positive rate of about 29%, In one example, the cutoff value is set for a positive rate of about 30%, In one example, the cutoff value is set for a positive rate of about 31%, In one example, the cutoff value is set for a positive rate of about 32%, In one example, the cutoff value is set for a positive rate of about 33%, In one example, the cutoff value is set for a positive rate of about 34%, In one example, the cutoff value is set for a positive rate of about 35%, In one example, the cutoff value is set for a positive rate of about 36%, In one example, the cutoff value is set for a positive rate of about 37%, In one example, the cutoff value is set for a positive rate of about 38%, In one example, the cutoff value is set for a positive rate of about 39%, In one example, the cutoff value is set for a positive rate of about 40%, In one example, the cutoff value is set for a positive rate of about 41%, In one example, the cutoff value is set for a positive rate of about 42%, In one example, the cutoff value is set for a positive rate of about 43%, In one example, the cutoff value is set for a positive rate of about 44%, In one example, the cutoff value is set for a positive rate of about 45%.

As is clear from the previous paragraph, the cutoff can be determined to achieve any positive predictive value, specificity, sensitivity, negative predictive value, positive rate, or accuracy. Accordingly, in some embodiments of the various methods provided herein, including in Sections 2, 5 (including in the paragraphs of Section 5.1), and 7, the cutoff in the methods is such a cutoff that provide any one of the positive predictive values provided in Sections 2, 5 (including in the paragraphs of Section 5.1), and 7. In some embodiments of the various methods provided herein, including in Sections 2, S (including in the paragraphs of Section 5.1), and 7, the cutoff in the methods is such a cutoff that provide any one of the specificity provided in Sections 2, 5 (including in the paragraphs of Section 5.1), and 7. In some embodiments of the various methods provided herein, including in Sections 2, 5 (including in the paragraphs of Section 5.1), and 7, the cutoff in the methods is such a cutoff that provide any one of the sensitivity provided in Sections 2, 5 (including in the paragraphs of Section 5.1), and 7. In some embodiments of the various methods provided herein, including in Sections 2, 5 (including in the paragraphs of Section 5.1), and 7, the cutoff in the methods is such a cutoff that provide any one of the negative predictive values provided in Sections 2, 5 (including in the paragraphs of Section 5.1), and 7. In some embodiments of the various methods provided herein, including in Sections 2, 5 (including in the paragraphs of Section 5.1), and 7, the cutoff in the methods is such a cutoff that provide any one of the accuracy provided in Sections 2, 5 (including in the paragraphs of Section 5.1), and 7. In some embodiments of the various methods provided herein, including in Sections 2, 5 (including in the paragraphs of Section 5.1), and 7, the cutoff in the methods is such a cutoff that provide any one of the positive rates provided in Sections 2, 5 (including in the paragraphs of Section 5.1), and 7.

In some embodiments of the methods provided herein, the step of determining a positive result is performed using a system selected from the systems provided in Section 5.6. In some embodiments of the methods provided herein (e.g. in this paragraph), the step of determining a positive result is performed using a computer implemented method selected from the computer implemented methods provided in Section 5.6. In some embodiments of the methods provided herein (e.g. in this paragraph), the step of determining TL1A isoforms, TL1A isoform ratio, or proxy markers is performed as described in Sections 2, 5.1, and 7. In some embodiments of the methods provided herein (e.g. in this paragraph), the TL1A isoform ratio is calculated as described in Sections 2, 5.1, and 7.

In one embodiment of the methods provided herein, including in Sections 2, 5 (including in the paragraphs of Section 5.1), and 7, having a positive correlation coefficient is having a positive Pearson correlation coefficient. In one embodiment of the methods provided herein, including in Sections 2, 5 (including in the paragraphs of Section 5.1), and 7, having a positive correlation coefficient is having a positive Spearman correlation coefficient. In one embodiment of the methods provided herein, including in Sections 2, 5 (including in the paragraphs of Section 5.1), and 7, having a negative correlation coefficient is having a negative Pearson correlation coefficient. In one embodiment of the methods provided herein, including in Sections 2, 5 (including in the paragraphs of Section 5.1), and 7, having a negative correlation coefficient is having a negative Spearman correlation coefficient. In one embodiment of the methods provided herein, including in Sections 2, 5 (including in the paragraphs of Section 5.1), and 7, the correlation is determined by Pearson correlation coefficient. In one embodiment of the methods provided herein, including in Sections 2, 5 (including in the paragraphs of Section 5.1), and 7, the correlation is determined by Spearman correlation coefficient. In one embodiment of the methods provided herein, including in Sections 2, 5 (including in the paragraphs of Section 5.1), and 7, the correlation coefficient is Pearson correlation coefficient. In one embodiment of the methods provided herein, including in Sections 2, 5 (including in the paragraphs of Section 5.1), and 7, the correlation coefficient is Spearman correlation coefficient.

In one embodiment of the methods provided herein, including in Sections 2, 5 (including in the paragraphs of Section 5.1), and 7, the cutoff value in the method is such a value that maximizes the accuracy of predicting the therapeutic response to TL1A inhibitor treatment in a training subject cohort. In one embodiment of the methods provided herein, including in Sections 2, 5 (including in the paragraphs of Section 5.1), and 7, the cutoff value in the method is such a value that maximizes the PPV of predicting the therapeutic response to TL1A inhibitor treatment in a training subject cohort. In one embodiment of the methods provided herein, including in Sections 2, 5 (including in the paragraphs of Section 5.1), and 7, the cutoff value in the method is such a value that maximizes the NPV of predicting the therapeutic response to TL1A inhibitor treatment in a training subject cohort. In one embodiment of the methods provided herein, including in Sections 2, 5 (including in the paragraphs of Section 5.1), and 7, the cutoff value in the method is such a value that maximizes the sum of PPV and NPV of predicting the therapeutic response to TL1A inhibitor treatment in a training subject cohort. In one embodiment of the methods provided herein, including in Sections 2, 5 (including in the paragraphs of Section 5.1), and 7, the cutoff value in the method is such a value that maximizes the sum of specificity and sensitivity of predicting the therapeutic response to TL1A inhibitor treatment in a training subject cohort.

As described above, when the proxy marker has a negative correlation coefficient with TL1A isoform ratio, e.g. proxy marker=−1×(TL1A isoform ratio) or proxy marker=1/(TL1A isoform ratio), the proxy marker inversely correlate with (i) an increase in a level of TL1A protein expression in a sample obtained from a subject or patient, as compared to a reference level of TL1A protein expression (e.g., derived from a normal individual), and/or (ii) an increase of a positive therapeutic response in IBD patients to a treatment with the TL1A inhibitor as compared to the reference level of response in patients not selected by TL1A isoform ratio or the proxy marker. Therefore, when the proxy marker has a negative correlation coefficient with TL LA isoform ratio, the subject selected for the methods provided herein (including in Sections 2, 5.1, and 7) will be based on proxy marker below the cutoff, all else in the methods remaining identical. Accordingly, for every embodiment of method described herein (including Sections 2, 5.1, and 7) that provides TL1A isoform ratio above a cutoff, the disclosure provides a corresponding embodiment in which the element of TL1A isoform ratio above a cutoff is substituted by proxy marker below a cutoff wherein the proxy marker has a negative correlation coefficient with TL1A isoform ratio, while keeping all else in the methods identical. Additionally, for every embodiment of method described herein (including Sections 2, 5.1, and 7) that provides TL1A isoform ratio above a cutoff wherein the proxy marker has a positive correlation coefficient with TL1A isoform ratio, the disclosure provides a corresponding embodiment in which the element of TL1A isoform ratio above a cutoff wherein the proxy marker has a positive correlation coefficient with TL1A isoform ratio is substituted by proxy marker below a cutoff wherein the proxy marker has a negative correlation coefficient with TL1A isoform ratio, while keeping all else in the methods identical.

In various embodiments of the methods or the computer-implemented systems provided herein, such as those provided in Sections 2, 5 (including in the paragraphs of Section 5.1 and paragraphs of Section 5.6), and 7, the subject has been treated with an advanced IBD therapy prior to the treatment with the inhibitor of TL1A activity or expression. In some embodiments of the methods or the computer-implemented systems provided herein, including in this paragraph, the subject has been treated with 1, 2, or 3 advanced IBD therapies prior to the treatment with the inhibitor of TL1A activity or expression. In some embodiments of the methods or the computer-implemented systems provided herein, the subject has not been treated with an advanced IBD therapy prior to the treatment with the inhibitor of TL1A activity or expression. In some embodiments of the methods or the computer-implemented systems provided herein, including in this paragraph, the advanced IBD therapy comprises one or more selected from the group consisting of a biologic therapeutic agent for IBD, an SIP1 modulator, or a JAK inhibitor. In some embodiments of the methods or the computer-implemented systems provided herein, including in this paragraph, the biologic therapeutic agent for IBD comprises an anti-TNFα antibody, an anti-IL23 antibody, or an anti-integrin antibody. In some embodiments of the methods or the computer-implemented systems provided herein, including in this paragraph, the anti-TNFα comprises adalimumab, infliximab, golimumab, certolizumab, or etanercept. In some embodiments of the methods or the computer-implemented systems provided herein, including in this paragraph, the anti-IL23 antibody comprises ustekinumab, guselkumab, risankizumab, brazikumab, mirikizumab, tildrakizumab, or briakinumab. In some embodiments of the methods or the computer-implemented systems provided herein, including in this paragraph, the anti-integrin antibody comprises etrolizumab, vedolizumab, natalizumab, or ontamalimab. In some embodiments of the methods or the computer-implemented systems provided herein, including in this paragraph, the SIP1 modulator comprises fingolimod, siponimod, etrasimod, ozanimod, ponesimod, amiselimod, ceralifimod, or mocravimod. In some embodiments of the methods or the computer-implemented systems provided herein, including in this paragraph, the JAK inhibitor comprises tofacitinib, abrocitinib, baricitinib, upadacitinib, or filgotinib.

In various embodiments, provided herein is a method of treating inflammatory bowel disease (IBD) in a subject in need thereof, comprising: administering to the subject a therapeutically effective amount of an antibody or an antigen-binding fragment that specifically binds TL1A. In some embodiments of the methods provided herein, such as those provided in Sections 2, 5 (including in the paragraphs of Section 5.1), and 7, the subjects can be selected by various selection criteria described in Sections 2, 5 (including in the paragraphs of Section 5.1), and 7. In some embodiments of the methods provided herein, such as those provided in Sections 2, 5 (including in the paragraphs of Section 5.1), and 7, the subjects can be selected by various selection criteria described in Sections 2, S (including in the paragraphs of Section 5.1), and 7, and the inhibitor of TL1A activity or expression can be any one described in Section 2, 5.3 and 7. In some embodiments of the methods provided herein, such as those provided in Sections 2, 5 (including in the paragraphs of Section 5.1), and 7, the inhibitor of TL1A activity or expression can be any one described in Section 2, 5.3 and 7. In some embodiments, the anti-TL1A antibody comprises antibody A. In some embodiments, the anti-TL1A antibody comprises antibody B. In some embodiments, the anti-TL1A antibody comprises antibody C. In some embodiments, the anti-TL1A antibody comprises antibody D. In some embodiments, the anti-TL1A antibody comprises antibody E. In some embodiments, the anti-TL1A antibody comprises antibody F. In some embodiments, the anti-TL1A antibody comprises antibody G. In some embodiments, the anti-TL1A antibody comprises antibody I. In some embodiments, the anti-TL1A antibody comprises antibody H. In some embodiments, the anti-TL1A antibody comprises antibody A2. In some embodiments, the anti-TL1A antibody comprises antibody B2. In some embodiments, the anti-TL1A antibody comprises antibody C2. In some embodiments, the anti-TL1A antibody comprises antibody D2. In some embodiments, the anti-TL1A antibody comprises antibody E2. In some embodiments, the anti-TL1A antibody comprises antibody F2. In some embodiments, the anti-TL1A antibody comprises antibody G2. In some embodiments, the anti-TL1A antibody comprises antibody 12. In some embodiments, the anti-TL1A antibody comprises antibody H2. In certain embodiments, the anti-TL1A antibody comprises any one of the antibodies of Table 10. In some embodiments, the anti-TL1A antibody comprises antibody A217. In some embodiments, the anti-TL1A antibody comprises antibody A220. In some embodiments, the anti-TL1A antibody comprises antibody A223. In some embodiments, the anti-TL1A antibody comprises antibody A219. In some embodiments, the anti-TL1A antibody comprises antibody A221. In some embodiments, the anti-TL1A antibody comprises antibody A200. In some embodiments, the anti-TL1A antibody comprises antibody A213. In some embodiments, the anti-TL1A antibody comprises antibody A212. In some embodiments, the anti-TL1A antibody comprises antibody A107. In some embodiments, the anti-TL1A antibody comprises antibody A205. In some embodiments, the anti-TL1A antibody comprises antibody A211. In some embodiments, the anti-TL1A antibody comprises antibody A199. In some embodiments, the anti-TL1A antibody comprises antibody A15. In some embodiments, the anti-TL1A antibody comprises antibody A30. In some embodiments, the anti-TL1A antibody comprises antibody A100. In some embodiments, the anti-TL1A antibody comprises antibody A181. In some embodiments, the anti-TL1A antibody comprises antibody A129. In some embodiments, the anti-TL1A antibody comprises antibody A214. In some embodiments, the anti-TL1A antibody comprises antibody A216. In some embodiments, the anti-TL1A antibody comprises antibody A122. In some embodiments, the anti-TL1A antibody comprises antibody A222. In some embodiments, the anti-TL1A antibody comprises antibody A188. In some embodiments, the anti-TL1A antibody comprises antibody A203. In some embodiments, the anti-TL1A antibody comprises antibody A147. In some embodiments, the anti-TL1A antibody comprises antibody A127. In some embodiments, the anti-TL1A antibody comprises antibody A126. In some embodiments, the anti-TL1A antibody comprises antibody A160. In some embodiments, the anti-TL1A antibody comprises antibody A157. In some embodiments, the anti-TL1A antibody comprises antibody A159. In some embodiments, the anti-TL1A antibody comprises antibody A218. In some embodiments, the anti-TL1A antibody comprises antibody A158. In some embodiments, the anti-TL1A antibody comprises antibody A125. In some embodiments, the anti-TL1A antibody comprises antibody A103. In some embodiments, the anti-TL1A antibody comprises antibody A64. In some embodiments, the anti-TL1A antibody comprises antibody A67. In some embodiments, the anti-TL1A antibody comprises antibody A138. In some embodiments, the anti-TL1A antibody comprises antibody A68. In some embodiments, the anti-TL1A antibody comprises antibody A94. In some embodiments, the anti-TL1A antibody comprises antibody A110. In some embodiments, the anti-TL1A antibody comprises antibody A197. In some embodiments, the anti-TL1A antibody comprises antibody A112. In some embodiments, the anti-TL1A antibody comprises antibody A169. In some embodiments, the anti-TL1A antibody comprises antibody A173. In some embodiments, the anti-TL1A antibody comprises antibody A179. In some embodiments, the anti-TL1A antibody comprises antibody A148. In some embodiments, the anti-TL1A antibody comprises antibody A115. In some embodiments, the anti-TL1A antibody comprises antibody A149. In some embodiments, the anti-TL1A antibody comprises antibody A134. In some embodiments, the anti-TL1A antibody comprises antibody A113. In some embodiments, the anti-TL1A antibody comprises antibody A151. In some embodiments, the anti-TL1A antibody comprises antibody A96. In some embodiments, the anti-TL1A antibody comprises antibody A132. In some embodiments, the anti-TL1A antibody comprises antibody A196. In some embodiments, the anti-TL1A antibody comprises antibody A172. In some embodiments, the anti-TL1A antibody comprises antibody A75. In some embodiments, the anti-TL1A antibody comprises antibody A174. In some embodiments, the anti-TL1A antibody comprises antibody A109. In some embodiments, the anti-TL1A antibody comprises antibody A198. In some embodiments, the anti-TL1A antibody comprises antibody A170. In certain embodiments, the anti-TL1A antibody comprises any one of the antibodies of Tables 20-21. In some embodiments, the anti-TL1A antibody comprises antibody clone 34. In some embodiments, the anti-TL1A antibody comprises antibody 5C3D11. In some embodiments, the anti-TL1A antibody comprises antibody 9E12E5. In some embodiments, the anti-TL1A antibody comprises antibody AS12824. In some embodiments, the anti-TL1A antibody comprises antibody AS12823. In some embodiments, the anti-TL1A antibody comprises antibody AS12819. In some embodiments, the anti-TL1A antibody comprises antibody AS12816. In some embodiments, the anti-TL1A antibody comprises antibody AS12825. In some embodiments, the anti-TL1A antibody comprises antibody 12835. In some embodiments, the anti-TL1A antibody comprises antibody 18-7 S93E. In some embodiments, the anti-TL1A antibody comprises antibody 18-7. In some embodiments, the anti-TL1A antibody comprises antibody 18-7 S92D. In some embodiments, the anti-TL1A antibody comprises antibody 18-7 S92H. In some embodiments, the anti-TL1A antibody comprises antibody 18-7 S92N. In some embodiments, the anti-TL1A antibody comprises antibody 18-7 S92Q. In some embodiments, the anti-TL1A antibody comprises antibody 18-7 CDRv. In some embodiments, the anti-TL1A antibody comprises antibody 21-3. In some embodiments, the anti-TL1A antibody comprises antibody 21-3 V102K. In some embodiments, the anti-TL1A antibody comprises antibody 21-3 V102M. In some embodiments, the anti-TL1A antibody comprises antibody 21-3 V102Q. In some embodiments, the anti-TL1A antibody comprises antibody 21-3 V102 W. In some embodiments, the anti-TL1A antibody comprises antibody 21-3 CDRv. In some embodiments, the anti-TL1A antibody comprises antibody 21-3 CDRv. In some embodiments, the anti-TL1A antibody comprises antibody clone 2. In some embodiments, the anti-TL1A antibody comprises antibody clone 52. In some embodiments, the anti-TL1A antibody comprises antibody clone 46. In some embodiments, the anti-TL1A antibody comprises antibody clone 47. In some embodiments, the anti-TL1A antibody comprises antibody clone 14. In some embodiments, the anti-TL1A antibody comprises antibody clone 16L. In some embodiments, the anti-TL1A antibody comprises antibody clone 17L. In some embodiments, the anti-TL1A antibody comprises antibody clone 17L-1. In some embodiments, the anti-TL1A antibody comprises antibody clone 23. In some embodiments, the anti-TL1A antibody comprises antibody clone 53. In some embodiments, the anti-TL1A antibody comprises antibody clone E1. In some embodiments, the anti-TL1A antibody comprises antibody clone 3-17L V-A. In some embodiments, the anti-TL1A antibody comprises antibody clone 3-17L. In some embodiments, the anti-TL1A antibody comprises antibody clone L8mod. In some embodiments, the anti-TL1A antibody comprises antibody clone X-V. In some embodiments, the anti-TL1A antibody comprises antibody clone X. In some embodiments, the anti-TL1A antibody comprises antibody clone XL3-6. In some embodiments, the anti-TL1A antibody comprises antibody clone XL3-10. In some embodiments, the anti-TL1A antibody comprises antibody clone XL3-15. In some embodiments, the anti-TL1A antibody comprises antibody clone L3-13. In some embodiments, the anti-TL1A antibody comprises antibody clone H3-1. In some embodiments, the anti-TL1A antibody comprises antibody clone H2-2. In some embodiments, the anti-TL1A antibody comprises antibody clone H2-5. In some embodiments, the anti-TL1A antibody comprises antibody M1. In some embodiments, the anti-TL1A antibody comprises antibody M2. In some embodiments, the anti-TL1A antibody comprises antibody M3. In some embodiments, the anti-TL1A antibody comprises antibody M4. In some embodiments, the anti-TL1A antibody comprises antibody M5. In some embodiments, the anti-TL1A antibody comprises antibody M6. In some embodiments, the anti-TL1A antibody comprises antibody M7. In some embodiments, the anti-TL1A antibody comprises antibody M8. In some embodiments, the anti-TL1A antibody comprises antibody M9. In some embodiments, the anti-TL1A antibody comprises antibody M10. In some embodiments, the anti-TL1A antibody comprises antibody M11. In some embodiments, the anti-TL1A antibody comprises antibody M12.

Methods disclosed herein provide methods of treating an inflammatory bowel disease (IBD) in a subject by administering an anti-TL1A antibody described herein to the subject. In various embodiments, IBD is Crohn's Disease (CD) or ulcerative colitis (UC). In some embodiments, the IBD is a severe form of IBD. In some embodiments, the IBD is a moderate to severe form of IBD. In some embodiments, the IBD is a moderate form of IBD. In various other embodiments, the subject is determined to have an increased TL1A expression. In some embodiments, the administration of a therapeutically effective amount of an anti-TL1A antibody causes a decrease in TL1A in the subject treated.

### 5.2 Methods of Detection

Methods disclosed herein for detecting TL1A isoform in a sample from a subject comprise analyzing the nucleic acid materials, including DNA and/or RNA, in the sample to detect and determine the level of TL1Ashort isoform, TL1A long isoform, and/or proxy markers, and administering an anti-TL1A antibody or antigen binding fragment as disclosed herein. In some embodiments, the sample is assayed to measure a presence, absence or quantity of TL1A short isoform, TL1A long isoform, and/or proxy markers. In some embodiments, the TL1Ashort isoform, TL1A long isoform, and/or proxy markers are detected using the methods described herein.

In some cases, the nucleic acid materials used for detecting and determining the level of TL1Ashort isoform, TL1A long isoform, and/or proxy markers comprises RNA. In some cases, the nucleic acid materials used for detecting and determining the level of TL1Ashort isoform. TL1A long isoform, and/or proxy markers comprises messenger RNA (mRNA). In some cases, the nucleic acid materials used for detecting and determining the level of TL1Ashort isoform, TL1A long isoform, and/or proxy markers comprises fragmented RNA. In some cases, the nucleic acid materials used for detecting and determining the level of TL1Ashort isoform. TL1A long isoform, and/or proxy markers comprises partially degraded RNA. In some cases, the nucleic acid materials used for detecting and determining the level of TL1Ashort isoform, TL1A long isoform, and/or proxy markers comprises microRNA. In some cases, the nucleic acid materials used for detecting and determining the level of TL1Ashort isoform, TL1A long isoform, and/or proxy markers comprises fragmented RNA. In some cases, the nucleic acid materials used for detecting and determining the level of TL1Ashort isoform, TL1A long isoform, and/or proxy markers comprises an RNA molecule or a fragmented RNA molecule (RNA fragments) selected from: a microRNA (miRNA), a pre-miRNA, a pri-miRNA, a mRNA, a pre-mRNA, circular RNA (circRNA), a long non-coding RNA (lncRNA), a small nuclear RNA (snRNA), a circulating RNA, a cell-free RNA, and/or an exosomal RNA. In some cases, the nucleic acid materials used for detecting and determining the level of TL1Ashort isoform, TL1A long isoform, and/or proxy markers comprises DNA. In some cases, the nucleic acid materials used for detecting and determining the level of TL1Ashort isoform, TL1A long isoform, and/or proxy markers comprises genomic DNA, plasmid DNA, amplified DNA, circular DNA, circulating DNA, cell-free DNA, or exosomal DNA.

Nucleic acid-based detection and quantification techniques that may be useful for the methods herein include next generation sequencing (NGS, including DNA sequencing and RNA sequencing, “RNAseq”), quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

The gene expression level (e.g. mRNA level) of TL1Ashort isoform, TL1A long isoform, and/or proxy markers can be detected, quantitated, and both detected and quantitated via NGS (e.g. RNAseq). The use of NGS to detect the nucleic acid reporters generated by assay methods disclosed herein include at least the following advantages. First. NGS is capable of detecting and reading the sequences of nucleic acid molecules in the sample, thereby specifically detect and/or quantitate TL1Ashort isoform and/or long isoform. Second, NGS can count and aggregate the number of molecules of the same sequence, providing digital quantification at single molecule resolution. Accordingly, TL1Ashort isoform, TL1A long isoform, and/or proxy markers can be specifically and quantitatively detected and determined via NGS (e.g. RNAseq). Such NGS (e.g. RNAseq) can be performed with commercially available instruments and according to manufacturer's instructions (e.g. from ILLUMINA). Additionally, a wide range of error correction algorithms, such as parity check, Hamming codes (e.g. Bystrykh, PLOS ONE 7(5): e36852 (2012)), and Levenshtein codes (e.g. Buschmann, BMC Bioinformatics. 2013; 14:272 (2013)) can be used from communication theory and applied herein to reduce false positive counts so that NGS based quantification can achieve high precision without repeated sequencing.

Since NGS is a single molecule detection and counting method, the sequencing instrument imposes an upper limit on the total number of molecules that can be sequenced. The MiSeq system from Illumina, for example, is capable of 25 million reads per run, which limits the total number of molecules to be sequenced in a run to 25 million. The inventors recognize that due to the mRNA transcript level of TL1Ashort isoform, the mRNA encoding TL1Ashort isoform is often missed in RNAseq analyses of lower sequencing depth. To detect and quantitate TL1Ashort isoform and the TL1A isoform ratio, the sequencing depth need to be increased 50 million reads. Accordingly, in some embodiment, the gene expression level (e.g. mRNA level) of TL1Ashort and long isoform is determined via RNAseq at a sequencing depth of at least 50 million reads. In some embodiment, the gene expression level (e.g. mRNA level) of TL1Ashort isoform is determined via RNAseq at a sequencing depth of at least 50 million reads. In some embodiment, the gene expression level (e.g. mRNA level) of TL1A long isoform is determined via RNAseq at a sequencing depth of at least 50 million reads. In some embodiment, the gene expression level (e.g. mRNA level) of TL1Ashort and long isoform is determined via RNAseq. In some embodiment, the gene expression level (e.g. mRNA level) of TL1Ashort isoform is determined via RNAseq. In some embodiment, the gene expression level (e.g. mRNA level) of TL1A long isoform is determined via RNAseq. In some embodiment, the gene expression level (e.g. mRNA level) of the proxy marker is determined via RNAseq at a sequencing depth of at least 50 million reads. In some embodiment, the gene expression level (e.g. mRNA level) of the proxy marker is determined via RNAseq.

In some instances, the methods involve hybridization and/or amplification assays that include, but are not limited to, Southern or Northem analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of TL1A isoform provided herein. A non-limiting method is one employed in Anal Chem. 2013 Feb. 5; 85(3):1932-9.

Numerous different PCR or qPCR protocols are known in the art and exemplified herein below and can be directly applied or adapted for determining the gene expression levels (e.g. mRNA levels) of TL1A isoforms and/or proxy markers in the samples or controls. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005); D′haene et al., Methods 50:262-270 (2010)), which is incorporated by reference herein in its entirety. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the TL1A isoforms and/or proxy markers mRNA levels as follows. Briefly, mean Ct (cycle threshold) values (or referred to herein interchangeably as Cq (quantification cycle)) of replicate qPCR reactions for (i) TL1A isoforms and/or proxy markers and (ii) one or more housekeeping genes are determined. Mean Ct values for TL1A isoforms and/or proxy markers can be then normalized to the Ct values of the housekeeping genes using the following exemplary formula: ΔCt=(mean Ct of TL1A isoforms and/or proxy markers—mean Ct of housekeeping gene A). The relative ΔCt can then be used to determine relative level of mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art for the quantification of mRNA expression in a sample can also be used, including northern blotting and in situ hybridization (Parker & Barnes, Methods in Molecular Biology 106:247-283 (1999)); RNAse protection assays (Hod, Biotechniques 13:852-854 (1992)); microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). Alternatively, levels of mRNA expression can be determined by sequencing techniques. Representative methods for sequencing-based gene expression analysis include Serial Analysis of Gene Expression (SAGE), and gene expression analysis by massively parallel signature sequencing (MPSS).

The samples from the subject used in the various methods can be, but are not limited to, tissues, bodily fluid, tissue fractions, and/or cells isolated from an organism such as a mammal, in particular a human. Therefore, the sample from the subject, e.g. tissue samples from the subject, can be obtained from a variety of organs of a subject, including a human subject. In some embodiments, tissue samples are obtained from organs suspected of having a disease, dysfunction or disorder, such as IBD (e.g. UC or CD). In other embodiments, tissue samples are obtained from normal organs from the patient who is being tested or from a second human subject and used as control. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

Expression or TL1A isoforms (long and short) and/or proxy markers can be detected by a variety of immunoassays known in the art, including an IHC approach, an immunoblotting assay, a FACS assay, and an ELISA, as further described below.

Expression or TL1A isoforms (long and short) and/or proxy markers can be detected by an antibody in a variety of IHC approaches. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target TL1A isoforms (long and short) or proxy markers, can be used to detect expression in an IHC assay. In some embodiments, the tissue sample is contacted with a primary antibody for a specific target for a period of time sufficient for the antibody-target binding to occur. The antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of target proteins such as TL1A isoforms (long and short) and/or proxy markers in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3, 3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “′spine” molecule of dextran to which 1, 2, 3, 4, 5, 6, 7, 8, 9 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system, HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting amplification of the number of labels at the site with minimal diffusion-related loss of signal localization. Consequently, the signal can be amplified 1, 2, 3, 4, 5, 6, 7, 8, 9 10, 15, 20, 25, 30, 50, 75, or 100 folds. As known to a person skilled in the art, the labels on the tyramide can be any labels described herein, including fluorophores, enzymes, haptens, radioisotopes, and/or photophores. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, MA USA 02451).

Thus in some embodiments of the methods provided herein, the expression level of TL1A isoforms (long and short) and/or proxy markers is detected with a signal amplification system.

In some embodiments, the specimen is then counterstained to identify cellular and subcellular elements.

In some embodiments, the expression level of TL1A isoforms (long and short) and/or proxy markers can also be detected with antibodies described herein using an immunoblotting assay. In some embodiments of an immunoblotting assay proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target TL1A isoforms (long and short) or proxy markers, can be used to detect protein expression. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, MA USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabelled primary antibody followed by labeled secondary antibody as described herein.

In other embodiments, the expression levels of TL1A isoforms (long and short) and/or proxy markers can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target TL1A isoforms (long and short) or proxy markers, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorichrome-tagged antibody, which indicates the expression level of the target protein. Therefore, the expression level of surface proteins can be detected using antibodies against the target protein. Non-fluorescent cytoplasmic proteins can also be observed by staining permeablized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of TL1A isoforms (long and short) and/or proxy markers can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assays. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength, the fluorescence observed indicates the presence of the target protein of interest. Immunofluorescence and ELA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present invention.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′, 5,5′-tetramethylbenzidine (TMB), Chloronaphthol (4-CN) (4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (ABC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl) umbelliferyl phosphate for alkaline phosphatase; 4-Methylumbelliferyl phosphate bis(2-amino-2-methyl-1,3-propanediol), 4-Methylumbelliferyl phosphate bis(cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-a-D-galactopyranoside for a-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL: Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp RedIM, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-S-(or 6), lissamine rhodamine B, and the like; 7-nitrobenz-2-oxa-1,3-diazole (NBD); fluorescein and derivatives thereof; napthalenes such as dansyl(5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), and the like. Exemplary chromophores include, but are not limited to, phenolphthalein, malachite green, nitroaromatics such as nitrophenyl, diazo dyes, dabsyl(4-dimethylaminoazobenzene-4′-sulfonyl), and the like.

Methods well known to a person skilled in the art such as microscopy or spectroscopy can be utilized to quantitate chromogenic or fluorescent detectable signals associated with the bound primary or secondary antibodies.

When the proxy marker comprises or consists of single nucleotide polymorphism, determining the genotype of the SNP can be performed at the nucleic acid level by performing DNA sequencing, RNAseq, a reverse transcriptase polymerase chain reaction (RT-PCR) or a hybridization assay with oligonucleotides that are substantially complementary to portions of cDNA molecules of the DNA encompassing the nucleoposition of the SNP under conditions suitable for RNA-seq, RT-PCR or hybridization, as further described in the paragraphs below.

In some embodiments, detecting the genotype or the gene expression level comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLID sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange: N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocy anine (Cy A); 1H, 5H, 11H, 15H-Xantheno [2,3, 4-ij: 5,6, 7-i′j ′]diquinolizin-18-ium, 9-12 (or 4)-[[[6-12,5-dioxo-1-pyrrolidiny])oxy]-6-oxohexyl|amino|sulfonyl]-4 (or 2)-sulfophenyl]-2,3, 6,7, 12,13, 16, 17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay. PCT primers and probes can be designed with tools known and used in the art. For example, forward and reverse primers for regions containing SNPs can be designed by uploading the flanking sequences into the Thermofisher OligoPerfect Primer Designer tool. The primer set with longest amplicon can be selected for the forward and reverse primers. Flanking sequences of SNPs can be obtained from the NCBI dbSNP database. Probes can be designed with the Thermofisher SNP genotype tool. The resulting probe design from the SNP genotype tool can be then truncated to 10~20 nucleotide flanks for the final design. Non-limiting examples of primer pairs useful for detecting one or more polymorphisms described herein are provided in Table 2, below.

TABLE 2
Exemplary Primer Sequences

| | Forward | Reverse | Wt_Probe_ | Mut_Probe_ |
|---|---|---|---|---|
| rsID | Primer | Primer | Hex | MFA |
| rs6478109 | TGCTTCTGGAAG | TGAGGTTCAAAAT | TG+C +AG+A | TG+C +AGG |
| | TGAAAGT (SEQ | GACAGAGG (SEQ | +T+TG GGA | +TTG GGA (SEQ |
| | ID NO: 2301) | ID NO: 2302) | (SEQ ID NO: | ID NO: 2304) |
| | | | 2303) | |

“Wt_Probe_Hex” and “Mut_Probe_FAM” mean “Wild type_probes_tagged with HEX reporter dye” and “Mut_probe_tagged with FAM reporter dye”, respectively. “+” stands for LNA bases (Locked nucleotides), which are analogues that are modified at 2′-O, 4′-C and form a bridge. This bridge results in restricted base pairing giving room to adjust the Tm as needed between the probes. Thus, +A, +T, +C or +G signify A, T, G or C bases are added on the modified backbone.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water. Tris buffer, or Tris-EDTA buffer before subsequent analysis. In an exemplary embodiment, the nucleic acid material is extracted in water. In some cases, extraction does not comprise nucleic acid purification.

In the exemplary qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, the target nucleic acid is at least 10 contiguous nucleic acid molecules comprising a non-reference allele at nucleoposition of the polymorphism of rs6478109. In some embodiments, the target nucleic acid is at least 10 contiguous nucleic acid molecules comprising an “A” or a “G” allele at nucleoposition of the polymorphism of rs6478109. In some embodiments, detecting the at least 10 contiguous nucleic acid molecules comprising an “A” or a “G” allele at nucleoposition of the polymorphism of rs6478109 is sufficient to detect the polymorphism at rs6478109. The polymorphism detected may be heterozygous or homozygous.

To practice the methods and systems provided herein, genetic material may be extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

### 5.3 Therapeutic Agents

### 5.3.1 Antibodies

In one aspect, provided herein are antibodies and antigen-binding fragments. In some embodiments, an antibody comprises an antigen-binding fragment that refers to a portion of an antibody having antigenic determining variable regions of an antibody. Examples of antigen-binding fragments include, but are not limited to, Fab, Fab′, F(ab′)₂, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′) 2, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgAQ1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region. The humanized heavy chain variable domain may comprise IGHV1-46*02 framework with no or fewer than about 10, 9, 8, 7, 6, 5, 4, 3, 2, or I amino acid mutations. The humanized light chain variable domain may comprise IGKV3-20 framework with no or fewer than about 10, 9, 8, 7, 6, 5, 4, 3, 2, or 1 amino acid mutations.

In some embodiments, chimeric antibodies refer to antibodies wherein the sequence of the immunoglobulin molecule is derived from two or more species. As a non-limiting example, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January: 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg NR and Rees AR, “WAM: an improved algorithm for modelling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof.

In some embodiments, an antibody that specifically binds to a protein indicates that the antibody reacts or associates more frequently, more rapidly, with greater duration, with greater affinity, or with some combination of the above to the protein than with alternative substances, including unrelated proteins.

In some embodiments, the terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

In some embodiments, a protein such as an antibody described herein comprises a hydrophobic amino acid. Non-limiting exemplary hydrophobic amino acids include glycine (Gly), proline (Pro), phenylalanine (Phe), alanine (Ala), isoleucine (Ile), leucine (Leu), and valine (Val). In some embodiments, a protein such as an antibody described herein comprises a hydrophilic amino acid. Non-limiting exemplary hydrophilic amino acids include serine (Ser), threonine (Thr), aspartic acid (Asp), glutamic acid (Glu), cysteine (Cys), asparagine (Asn), glutamine (Gln), arginine (Arg), and histidine (His). In some embodiments, a protein such as an antibody described herein comprises an amphipathic amino acid. Non-limiting exemplary amphipathic amino acids include lysine (Lys), tryptophan (Trp), tyrosine (Tyr), and methionine (Met). In some embodiments, a protein such as an antibody described herein comprises an aliphatic amino acid. Non-limiting exemplary aliphatic amino acids include alanine (Ala), isoleucine (Ile), leucine (Leu) and valine (Val). In some embodiments, a protein such as an antibody described herein comprises an aromatic amino acid. Non-limiting exemplary aromatic amino acids include phenylalanine (Phe), tryptophan (Trp), and tyrosine (Tyr). In some embodiments, a protein such as an antibody described herein comprises an acidic amino acid. Non-limiting exemplary acidic amino acids include aspartic acid (Asp) and glutamic acid (Glu). In some embodiments, a protein such as an antibody described herein comprises a basic amino acid. Non-limiting exemplary basic amino acids include arginine (Arg), histidine (His), and lysine (Lys). In some embodiments, a protein such as an antibody described herein comprises a hydroxylic amino acid. Non-limiting exemplary hydroxylic amino acids include serine (Ser) and threonine (Thr). In some embodiments, a protein such as an antibody described herein comprises a sulfur-containing amino acid. Non-limiting exemplary sulfur-containing amino acids include cysteine (Cys) and methionine (Met). In some embodiments, a protein such as an antibody described herein comprises an amidic amino acid. Non-limiting exemplary amidic amino acids include asparagine (Asn) and glutamine (Gln).

In some embodiments, “polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXUS10087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the term “about” means within 10% of the stated amount. For instance, an antibody variable region comprising about 80% identity to a reference variable region may comprise 72% to 88% identity to the reference variable region.

In certain aspects, antibodies are described herein that specifically bind to TL1A (Entrez Gene: 9966; UniProtKB: 095150). In some embodiments, the antibodies specifically bind to soluble TL1A. In some embodiments, the antibodies specifically bind to membrane bound TL1A. In some embodiments, an anti-TL1A antibody is provided having a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. An anti-TL1A antibody may comprise any region provided herein, for example, as provided in Tables 15-21, the examples, and the sequences.

### 5.3.1.1 Exemplary Anti-TL1A CDRs

In certain embodiments, an anti-TL1A antibody comprises a HCDR1 as set forth by any one of SEQ ID NOS: 1 or 601-722. In certain embodiments, an anti-TL1A antibody comprises a HCDR2 as set forth by any one of SEQ ID NOS: 2-5 or 723-787. In certain embodiments, an anti-TL1A antibody comprises a HCDR3 as set forth by any one of SEQ ID NOS: 6-9 or 788-842. In certain embodiments, an anti-TL1A antibody comprises a LCDR1 as set forth by any one of SEQ ID NOS: 10 or 843-865. In certain embodiments, an anti-TL1A antibody comprises a LCDR2 as set forth by any one of SEQ ID NOS: 11 or 866-885. In certain embodiments, an anti-TL1A antibody comprises a LCDR3 as set forth by any one of SEQ ID NOS: 12-15 or 886-1101.

In one aspect, provided herein are anti-TL1A antibodies having a HCDR1 comprising any one of SEQ ID NOS: 1 or 601-708, a HCDR2 comprising any one of SEQ ID NOS: 2-5 or 723-774, a HCDR3 comprising any one of SEQ ID NOS: 6-9 or 788-828, a LCDR1 comprising any one of SEQ ID NOS: 10 or 843-852, a LCDR2 comprising any one of SEQ ID NOS: 11 or 866-873, and a LCDR3 comprising any one of SEQ ID NOS: 12-15 or 886-1089.

In one aspect, provided herein are anti-TL1A antibodies having a HCDR1 comprising SEQ ID NO: 709, a HCDR2 comprising SEQ ID NO: 775, a HCDR3 comprising SEQ ID NO: 829, a LCDR1 comprising SEQ ID NO: 853, a LCDR2 comprising SEQ ID NO: 874, and a LCDR3 comprising SEQ ID NO: 1090.

In one aspect, provided herein are anti-TL1A antibodies having a HCDR1 comprising SEQ ID NO: 710, a HCDR2 comprising SEQ ID NO: 776, a HCDR3 comprising SEQ ID NO: 830, a LCDR1 comprising SEQ ID NO: 854, a LCDR2 comprising SEQ ID NO: 875, and a LCDR3 comprising SEQ ID NO: 1091. In one aspect, provided herein are anti-TL1A antibodies having a HCDR1 comprising SEQ ID NO: 711 or 712, a HCDR2 comprising SEQ ID NO: 777 or 778, a HCDR3 comprising SEQ ID NO: 831 or 832, a LCDR1 comprising SEQ ID NO: 855, a LCDR2 comprising SEQ ID NO: 876, and a LCDR3 comprising SEQ ID NO: 1092.

In one aspect, provided herein are anti-TL1A antibodies having a HCDR1 comprising SEQ ID NO: 713, a HCDR2 comprising SEQ ID NO: 779, a HCDR3 comprising SEQ ID NO: 833, a LCDR1 comprising SEQ ID NO: 856, a LCDR2 comprising SEQ ID NO: 877, and a LCDR3 comprising SEQ ID NO: 1093.

In one aspect, provided herein are anti-TL1A antibodies having a HCDR1 comprising SEQ ID NO: 714, a HCDR2 comprising SEQ ID NO: 780, a HCDR3 comprising SEQ ID NO: 834, a LCDR1 comprising SEQ ID NO: 857, a LCDR2 comprising SEQ ID NO: 878, and a LCDR3 comprising SEQ ID NO: 1094.

In one aspect, provided herein are anti-TL1A antibodies having a HCDR1 comprising SEQ ID NO: 715 or 716, a HCDR2 comprising SEQ ID NO: 781, a HCDR3 comprising SEQ ID NO: 835 or 836, a LCDR1 comprising SEQ ID NO: 858 or 859, a LCDR2 comprising SEQ ID NO: 879, and a LCDR3 comprising SEQ ID NO: 1095.

In one aspect, provided herein are anti-TL1A antibodies having a HCDR1 comprising SEQ ID NO: 717, a HCDR2 comprising SEQ ID NO: 782, a HCDR3 comprising SEQ ID NO: 837, a LCDR1 comprising SEQ ID NO: 860, a LCDR2 comprising SEQ ID NO: 880, and a LCDR3 comprising SEQ ID NO: 1096.

In one aspect, provided herein are anti-TL1A antibodies having a HCDR1 comprising SEQ ID NO: 718, a HCDR2 comprising SEQ ID NO: 783, a HCDR3 comprising SEQ ID NO: 838, a LCDR1 comprising SEQ ID NO: 861, a LCDR2 comprising SEQ ID NO: 881, and a LCDR3 comprising SEQ ID NO: 1097.

In one aspect, provided herein are anti-TL1A antibodies having a HCDR1 comprising SEQ ID NO: 719, a HCDR2 comprising SEQ ID NO: 784, a HCDR3 comprising SEQ ID NO: 839, a LCDR1 comprising SEQ ID NO: 862, a LCDR2 comprising SEQ ID NO: 882, and a LCDR3 comprising SEQ ID NO: 1098.

In one aspect, provided herein are anti-TL1A antibodies having a HCDR1 comprising SEQ ID NO: 720, a HCDR2 comprising SEQ ID NO: 785, a HCDR3 comprising SEQ ID NO: 840, a LCDR1 comprising SEQ ID NO: 863, a LCDR2 comprising SEQ ID NO: 883, and a LCDR3 comprising SEQ ID NO: 1099.

In one aspect, provided herein are anti-TL1A antibodies having a HCDR1 comprising SEQ ID NO: 721, a HCDR2 comprising SEQ ID NO: 786, a HCDR3 comprising SEQ ID NO: 841, a LCDR1 comprising SEQ ID NO: 864, a LCDR2 comprising SEQ ID NO: 884, and a LCDR3 comprising SEQ ID NO: 1100.

In one aspect, provided herein are anti-TL1A antibodies having a HCDR1 comprising SEQ ID NO: 722, a HCDR2 comprising SEQ ID NO: 787, a HCDR3 comprising SEQ ID NO: 842, a LCDR1 comprising SEQ ID NO: 865, a LCDR2 comprising SEQ ID NO: 885, and a LCDR3 comprising SEQ ID NO: 1101.

In certain embodiments, an anti-TL1A antibody comprises a HCDR1, HCDR2, HCDR3, LCDR1, LCDR2, LCDR3 selected from Table 15. In certain embodiments, an anti-TL1A antibody comprises the CDRs set forth in antibody A as shown in Table 20. In certain embodiments, an anti-TL1A antibody comprises the CDRs set forth in antibody B as shown in Table 20. In certain embodiments, an anti-TL1A antibody comprises the CDRs set forth in antibody C as shown in Table 20. In certain embodiments, an anti-TL1A antibody comprises the CDRs set forth in antibody D as shown in Table 20. In certain embodiments, an anti-TL1A antibody comprises the CDRs set forth in antibody E as shown in Table 20. In certain embodiments, an anti-TL1A antibody comprises the CDRs set forth in antibody F as shown in Table 20. In certain embodiments, an anti-TL1A antibody comprises the CDRs set forth in antibody G as shown in Table 20. In certain embodiments, an anti-TL1A antibody comprises the CDRs set forth in antibody H as shown in Table 20. In certain embodiments, an anti-TL1A antibody comprises the CDRs set forth in antibody A2 as shown in Table 20. In certain embodiments, an anti-TL1A antibody comprises the CDRs set forth in antibody B2 as shown in Table 20. In certain embodiments, an anti-TL1A antibody comprises the CDRs set forth in antibody C2 as shown in Table 20. In certain embodiments, an anti-TL1A antibody comprises the CDRs set forth in antibody D2 as shown in Table 20. In certain embodiments, an anti-TL1A antibody comprises the CDRs set forth in antibody E2 as shown in Table 20. In certain embodiments, an anti-TL1A antibody comprises the CDRs set forth in antibody F2 as shown in Table 20. In certain embodiments, an anti-TL1A antibody comprises the CDRs set forth in antibody G2 as shown in Table 20. In certain embodiments, an anti-TL1A antibody comprises the CDRs set forth in antibody H2 as shown in Table 20. In certain embodiments, an anti-TL1A antibody comprises the CDRs set forth in antibody I as shown in Table 20. In certain embodiments, an anti-TL1A antibody comprises the CDRs set forth in antibody 12 as shown in Table 20. In certain embodiments, an anti-TL1A antibody comprises the CDRs set forth in antibody M1 as shown in Table 15. In certain embodiments, an anti-TL1A antibody comprises the CDRs set forth in antibody M2 as shown in Table 15. In certain embodiments, an anti-TL1A antibody comprises the CDRs set forth in antibody M3 as shown in Table 15. In certain embodiments, an anti-TL1A antibody comprises the CDRs set forth in antibody M4 as shown in Table 15. In certain embodiments, an anti-TL1A antibody comprises the CDRs set forth in antibody M5 as shown in Table 15. In certain embodiments, an anti-TL1A antibody comprises the CDRs set forth in antibody M6 as shown in Table 15. In certain embodiments, an anti-TL1A antibody comprises the CDRs set forth in antibody M7 as shown in Table 15. In certain embodiments, an anti-TL1A antibody comprises the CDRs set forth in antibody M8 as shown in Table 15. In certain embodiments, an anti-TL1A antibody comprises the CDRs set forth in antibody M9 as shown in Table 15. In certain embodiments, an anti-TL1A antibody comprises the CDRs set forth in antibody M10 as shown in Table 15. In certain embodiments, an anti-TL1A antibody comprises the CDRs set forth in antibody M11 as shown in Table 15. In certain embodiments, an anti-TL1A antibody comprises the CDRs set forth in antibody M12 as shown in Table 15. In certain embodiments, an anti-TL1A antibody comprises a P HCDR1 (any one of SEQ ID NOS: 1 or 601-708), a P HCDR2 (any one of SEQ ID NOS: 2-5 or 723-774), a P HCDR3 (any one of SEQ ID NOS: 6-9 or 788-828), a P LCDR1 (any one of SEQ ID NOS: 10 or 843-852), a P LCDR2 (any one of SEQ ID NOS: 11 or 866-873), and a P LCDR3 (any one of SEQ ID NOS: 12-15 or 886-1089) as shown in Table 15.

In certain embodiments, an anti-TL1A antibody comprises the CDRs set forth in any one of the antibodies in Table 10. In certain embodiments, an anti-TL1A antibody comprises the CDRs set forth in any one of the heavy chain variable regions in Table 16. In certain embodiments, an anti-TL1A antibody comprises the CDRs set forth in any one of the light chain variable regions in Table 17. The CDRs may be defined by the Aho or Kabat, Chothia, or IMGT method. In certain embodiments, an anti-TL1A antibody comprises the CDRs set forth in antibody A217. In certain embodiments, an anti-TL1A antibody comprises the CDRs set forth in antibody A220. In certain embodiments, an anti-TL1A antibody comprises the CDRs set forth in antibody A223. In certain embodiments, an anti-TL1A antibody comprises the CDRs set forth in antibody A219. In certain embodiments, an anti-TL1A antibody comprises the CDRs set forth in antibody A221. In certain embodiments, an anti-TL1A antibody comprises the CDRs set forth in antibody A200. In certain embodiments, an anti-TL1A antibody comprises the CDRs set forth in antibody A213. In certain embodiments, an anti-TL1A antibody comprises the CDRs set forth in antibody A212. In certain embodiments, an anti-TL1A antibody comprises the CDRs set forth in antibody A107. In certain embodiments, an anti-TL1A antibody comprises the CDRs set forth in antibody A205. In certain embodiments, an anti-TL1A antibody comprises the CDRs set forth in antibody A211. In certain embodiments, an anti-TL1A antibody comprises the CDRs set forth in antibody A199. In certain embodiments, an anti-TL1A antibody comprises the CDRs set forth in antibody A15. In certain embodiments, an anti-TL1A antibody comprises the CDRs set forth in antibody A30. In certain embodiments, an anti-TL1A antibody comprises the CDRs set forth in antibody A100. In certain embodiments, an anti-TL1A antibody comprises the CDRs set forth in antibody A181. In certain embodiments, an anti-TL1A antibody comprises the CDRs set forth in antibody A129. In certain embodiments, an anti-TL1A antibody comprises the CDRs set forth in antibody A214. In certain embodiments, an anti-TL1A antibody comprises the CDRs set forth in antibody A216. In certain embodiments, an anti-TL1A antibody comprises the CDRs set forth in antibody A122. In certain embodiments, an anti-TL1A antibody comprises the CDRs set forth in antibody A222. In certain embodiments, an anti-TL1A antibody comprises the CDRs set forth in antibody A188. In certain embodiments, an anti-TL1A antibody comprises the CDRs set forth in antibody A203. In certain embodiments, an anti-TL1A antibody comprises the CDRs set forth in antibody A147. In certain embodiments, an anti-TL1A antibody comprises the CDRs set forth in antibody A127. In certain embodiments, an anti-TL1A antibody comprises the CDRs set forth in antibody A126. In certain embodiments, an anti-TL1A antibody comprises the CDRs set forth in antibody A160. In certain embodiments, an anti-TL1A antibody comprises the CDRs set forth in antibody A157. In certain embodiments, an anti-TL1A antibody comprises the CDRs set forth in antibody A159. In certain embodiments, an anti-TL1A antibody comprises the CDRs set forth in antibody A218. In certain embodiments, an anti-TL1A antibody comprises the CDRs set forth in antibody A158. In certain embodiments, an anti-TL1A antibody comprises the CDRs set forth in antibody A125. In certain embodiments, an anti-TL1A antibody comprises the CDRs set forth in antibody A103. In certain embodiments, an anti-TL1A antibody comprises the CDRs set forth in antibody A64. In certain embodiments, an anti-TL1A antibody comprises the CDRs set forth in antibody A67. In certain embodiments, an anti-TL1A antibody comprises the CDRs set forth in antibody A138. In certain embodiments, an anti-TL1A antibody comprises the CDRs set forth in antibody A68. In certain embodiments, an anti-TL1A antibody comprises the CDRs set forth in antibody A94. In certain embodiments, an anti-TL1A antibody comprises the CDRs set forth in antibody A110. In certain embodiments, an anti-TL1A antibody comprises the CDRs set forth in antibody A197. In certain embodiments, an anti-TL1A antibody comprises the CDRs set forth in antibody A112. In certain embodiments, an anti-TL1A antibody comprises the CDRs set forth in antibody A169. In certain embodiments, an anti-TL1A antibody comprises the CDRs set forth in antibody A173. In certain embodiments, an anti-TL1A antibody comprises the CDRs set forth in antibody A179. In certain embodiments, an anti-TL1A antibody comprises the CDRs set forth in antibody A148. In certain embodiments, an anti-TL1A antibody comprises the CDRs set forth in antibody A115. In certain embodiments, an anti-TL1A antibody comprises the CDRs set forth in antibody A149. In certain embodiments, an anti-TL1A antibody comprises the CDRs set forth in antibody A134. In certain embodiments, an anti-TL1A antibody comprises the CDRs set forth in antibody A113. In certain embodiments, an anti-TL1A antibody comprises the CDRs set forth in antibody A151. In certain embodiments, an anti-TL1A antibody comprises the CDRs set forth in antibody A96. In certain embodiments, an anti-TL1A antibody comprises the CDRs set forth in antibody A132. In certain embodiments, an anti-TL1A antibody comprises the CDRs set forth in antibody A196. In certain embodiments, an anti-TL1A antibody comprises the CDRs set forth in antibody A172. In certain embodiments, an anti-TL1A antibody comprises the CDRs set forth in antibody A75. In certain embodiments, an anti-TL1A antibody comprises the CDRs set forth in antibody A174. In certain embodiments, an anti-TL1A antibody comprises the CDRs set forth in antibody A109. In certain embodiments, an anti-TL1A antibody comprises the CDRs set forth in antibody A198. In certain embodiments, an anti-TL1A antibody comprises the CDRs set forth in antibody A170. In certain embodiments, an anti-TL1A antibody comprises the CDRs set forth in antibody M1. In certain embodiments, an anti-TL1A antibody comprises the CDRs set forth in antibody M2. In certain embodiments, an anti-TL1A antibody comprises the CDRs set forth in antibody M3. In certain embodiments, an anti-TL1A antibody comprises the CDRs set forth in antibody M4. In certain embodiments, an anti-TL1A antibody comprises the CDRs set forth in antibody M5. In certain embodiments, an anti-TL1A antibody comprises the CDRs set forth in antibody M6. In certain embodiments, an anti-TL1A antibody comprises the CDRs set forth in antibody M7. In certain embodiments, an anti-TL1A antibody comprises the CDRs set forth in antibody M8. In certain embodiments, an anti-TL1A antibody comprises the CDRs set forth in antibody M9. In certain embodiments, an anti-TL1A antibody comprises the CDRs set forth in antibody M10. In certain embodiments, an anti-TL1A antibody comprises the CDRs set forth in antibody M11. In certain embodiments, an anti-TL1A antibody comprises the CDRs set forth in antibody M12.

### 5.3.1.2 Exemplary Anti-TL1A Framework Regions

Tables 16-18 and Table 21 provides exemplary framework and variable region sequences. In some embodiments, an anti-TL1A antibody comprises a HC FR1 of Table 19 (SEQ ID NO: 304), a HC FR2 of Table 19 (any one of SEQ ID NOS: 305, 313, 1317), a HC FR3 of Table 19 (any one of SEQ ID NOS: 306, 307, 314, 315, 1318-1323), a HC FR4 of Table 19 (SEQ ID NO: 308), a LC FR1 of Table 19 (SEQ ID NO: 309), a LC FR2 of Table 19 (SEQ ID NO: 310 or 1324), a LC FR3 of Table 19 (SEQ ID NO: 311), and a LC FR4 of Table 19 (SEQ ID NO: 312).

In some embodiments, an anti-TL1A antibody comprises a heavy chain framework comprising SEQ ID NO: 301 (XIVQLVQSGAEVKKPGASVKVSCKAS[HCDR1]WVX2QX3PGQGLEWX4G[HCDR2]RX STX6TX7DTSTSTX8YX9ELSSLRSEDTAVYYCAR[HCDR3]WGQGTTVTVSS). In some cases, X1 is Q. In some cases, X1=E. In some cases, X2=R. In some cases, X2=K. In some cases. X3=A. In some cases, X3=R. In some cases, X4=M. In some cases, X4=1. In some cases. X5=V. In some cases, X5=A. In some cases, X6=M. In some cases, X6=I. In some cases, X7=R. In some cases, X7=T. In some cases, X8=V. In some cases, X8=A. In some cases, X9=M. In some cases, X9=L. In some embodiments, X1 is at position 1 of IGHV1-46*02 as determined by Aho or Kabat numbering. In some embodiments, X2 is at position 45 of IGHV1-46*02 as determined by Aho or Kabat numbering. In some embodiments, X3 is at position 47 of IGHV1-46*02 as determined by Aho or Kabat numbering. In some embodiments, X4 is at position 55 of IGHV1-46*02 as determined by Aho or Kabat numbering. In some embodiments, X5 is at position 78 of IGHV1-46*02 as determined by Aho or Kabat numbering. In some embodiments, X6 is at position 80 of IGHV1-46*02 as determined by Aho or Kabat numbering. In some embodiments. X7 is at position 82 of IGHV 1-46*02 as determined by Aho or Kabat numbering. In some embodiments, X8 is at position 89 of IGHV1-46*02 as determined by Aho or Kabat numbering. In some embodiments, X9 is at position 91 of IGHV1-46*02 as determined by Aho or Kabat numbering.

In one aspect, provided herein is a first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 301. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (29) The anti-TL1A of any one of embodiments 1-28, comprising a human IgG1 Fc region comprising. (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (1) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K. (v) 269N or 269Q, (w) 270A, 270G, 270M, or 270N, (x) 271T, (y) 272N. (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 37GW, or 376Y, (II) 380D, (mm) 382D or 382P, (nn) 385P, (co) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S. H268A, A330S, and P331S (IgGlo), (ccc) L234A, L235A, and P329A, (ddd) G236R and L328R, (eee) G237A, (fff) F241A, (ggg) V264A, (hhh) D265A. (iii) D265A and N297A, (jj) D265A and N297G, (kkk) D270A. (III) A330L, (mmm) P331A or P331S, or (nnn) any combination of (a)-(uu), per Kabat numbering. As used herein, any combination of a group, such as (a) to (uu), includes at least about two or more items from the group, e.g., any combination of a group of (a) to (uu) includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and up to 47 or all of the members of the group. (30) The anti-TL1A of any one of embodiments 1-28, comprising a (i) human IgG4 Fc region or (ii) a human IgG4 Fc region comprising (a) S228P, (b) S228P and L235E, or (c) S228P. F234A, and L235A, per Kabat numbering. (31) The anti-TL1A of any one of embodiments 1-28, comprising a human IgG2 Fc region; lgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG20). (32) The anti-TL1A of any one of embodiments 1-31, comprising a heavy chain Fc region comprising any one of SEQ ID NOS: 320-362. (33) The anti-TL1A of any one of embodiments 1-32, comprising a light chain constant region comprising SEQ ID NO: 319. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X11 is L. (38) The anti-TL1A antibody of any one of embodiments 34-36, wherein X11 is W.

In some embodiments, an anti-TL1A antibody comprises a heavy chain framework comprising SEQ ID NO: 302 (XIVQLVQSGAEVKKPGASVKVSCKAS[HCDR1]WVX2QX3PGQGLEWX4G[HCDR2]RX STX6TX7DTSTSTX8YX9ELSSLRSEDTAVYYC[HCDR3]WGQGTTVTVSS). In some cases, X1 is Q. In some cases, X1=E. In some cases. X2=R. In some cases, X2=K. In some cases, X3=A. In some cases, X3=R. In some cases, X4=M. In some cases, X4=I. In some cases, X5=V. In some cases, X5=A. In some cases, X6=M. In some cases, X6=I. In some cases, X7=R. In some cases. X7=T. In some cases, X8=V. In some cases, X8=A. In some cases, X9=M. In some cases, X9=L. In some embodiments, X1 is at position 1 of IGHV1-46*02 as determined by Aho or Kabat numbering. In some embodiments, X2 is at position 45 of IGHV1-46*02 as determined by Aho or Kabat numbering. In some embodiments, X3 is at position 47 of IGHV1-46*02 as determined by Aho or Kabat numbering. In some embodiments, X4 is at position 55 of IGHV1-46*02 as determined by Aho or Kabat numbering. In some embodiments, X5 is at position 78 of IGHV1-46*02 as determined by Aho or Kabat numbering. In some embodiments, X6 is at position 80 of IGHV1-46*02 as determined by Aho or Kabat numbering. In some embodiments, X7 is at position 82 of IGHV1-46*02 as determined by Aho or Kabat numbering. In some embodiments, X8 is at position 89 of IGHV 1-46*02 as determined by Aho or Kabat numbering. In some embodiments. X9 is at position 91 of IGHV1-46*02 as determined by Aho or Kabat numbering.

In one aspect, provided herein is another first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 302. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (29) The anti-TL1A of any one of embodiments 1-28, comprising a human IgG1 Fc region comprising: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 2671, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (cee) G237A. (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. (30) The anti-TL1A of any one of embodiments 1-28, comprising a (i) human IgG4 Fc region or (ii) a human IgG4 Fc region comprising (a) S228P and L235E, or (b) S228P. F234A, and L235A, per Kabat numbering. (31) The anti-TL1A of any one of embodiments 1-28, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG26). (32) The anti-TL1A of any one of embodiments 1-31, comprising a heavy chain Fc region comprising any one of SEQ ID NOS: 320-362. (33) The anti-TL1A of any one of embodiments 1-32, comprising a light chain constant region comprising SEQ ID NO: 319. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X11 is L. (38) The anti-TL1A antibody of any one of embodiments 34-36, wherein X11 is W.

In some embodiments, an anti-TL1A antibody comprises a light chain framework comprising SEQ ID NO: 303 (EIVLTQSPGTLSLSPGERATLSC[LCDR1]WYQQKPGQAPRX10X11IY [LCDR2]GIPDRFS GSGSGTDFTLTISRLEPEDFAVYYC[LCDR3]FGGGTKLEIK). In some cases, X10 is L. In some cases, X10 is P. In some cases, X11 is L. In some cases, X11 is W. In some embodiments, X10 is at position 54 of IGKV3-20*01 as determined by Aho or Kabat numbering. In some embodiments, X11 is at position 55 of IGKV3-20*01 as determined by Aho or Kabat numbering.

In some embodiments, an anti-TL1A antibody comprises a heavy chain framework comprising IGHV1-46*02. In some embodiments, an anti-TL1A antibody comprises a heavy chain framework comprising a variant of IGHV1-46*02 comprising between about 1 and about 20 amino acid substitutions from SEQ ID NO: 316. In some embodiments, an anti-TL1A antibody comprises a heavy chain framework comprising a variant of IGHV1-46*02 comprising between about 1 and about 9 amino acid substitutions from SEQ ID NO: 316. In some embodiments, an anti-TL1A antibody comprises a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. In some cases, the heavy chain framework substitution comprises Q1E, as determined by Aho or Kabat numbering. In some cases, the heavy chain framework substitution comprises R45K, as determined by Aho or Kabat numbering. In some cases, the heavy chain framework substitution comprises A47R, as determined by Aho or Kabat numbering. In some cases, the heavy chain framework substitution comprises M55I, as determined by Aho or Kabat numbering. In some cases, the heavy chain framework substitution comprises V78A, as determined by Aho or Kabat numbering. In some cases, the heavy chain framework substitution comprises M80I, as determined by Aho or Kabat numbering. In some cases, the heavy chain framework substitution comprises R82T, as determined by Aho or Kabat numbering. In some cases, the heavy chain framework substitution comprises V89A, as determined by Aho or Kabat numbering. In some cases, the heavy chain framework substitution comprises M91L, as determined by Aho or Kabat numbering.

In some embodiments, an anti-TL1A antibody comprises a light chain framework comprising IGKV3-20*01. In some embodiments, an anti-TL1A antibody comprises a variant of IGKV3-20*01 comprising between about 1 and about 20 amino acid substitutions from SEQ ID NO: 317. In some embodiments, an anti-TL1A antibody comprises a variant of IGKV3-20*01 comprising about 1 amino acid substitution from SEQ ID NO: 317. In some embodiments, an anti-TL1A antibody comprises a light chain framework comprising a variant of IGKV3-20*01 comprising about 2 amino acid substitutions from SEQ ID NO: 317. In some embodiments, an anti-TL1A antibody comprises a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. In some cases, the light chain framework substitution comprises Q1E, as determined by Aho or Kabat numbering. In some cases, the light chain framework substitution comprises R45K, as determined by Aho or Kabat numbering.

In some embodiments, an anti-TL1A antibody comprises a heavy chain FR1 as set forth by SEQ ID NO: 304. In some embodiments, an anti-TL1A antibody comprises a heavy chain FR2 as set forth by SEQ ID NO: 305. In some embodiments, an anti-TL1A antibody comprises a heavy chain FR2 as set forth by SEQ ID NO: 313. In some embodiments, an anti-TL1A antibody comprises a heavy chain FR2 as set forth by SEQ ID NO: 1317. In some embodiments, an anti-TL1A antibody comprises a heavy chain FR3 as set forth by SEQ ID NO: 306. In some embodiments, an anti-TL1A antibody comprises a heavy chain FR3 as set forth by SEQ ID NO: 307. In some embodiments, an anti-TL1A antibody comprises a heavy chain FR3 as set forth by SEQ ID NO: 314. In some embodiments, an anti-TL1A antibody comprises a heavy chain FR3 as set forth by SEQ ID NO: 315. In some embodiments, an anti-TL1A antibody comprises a heavy chain FR3 as set forth by SEQ ID NO: 1318. In some embodiments, an anti-TL1A antibody comprises a heavy chain FR3 as set forth by SEQ ID NO: 1319. In some embodiments, an anti-TL1A antibody comprises a heavy chain FR3 as set forth by SEQ ID NO: 1320. In some embodiments, an anti-TL1A antibody comprises a heavy chain FR3 as set forth by SEQ ID NO: 1321. In some embodiments, an anti-TL1A antibody comprises a heavy chain FR3 as set forth by SEQ ID NO: 1322. In some embodiments, an anti-TL1A antibody comprises a heavy chain FR3 as set forth by SEQ ID NO: 1323. In some embodiments, an anti-TL1A antibody comprises a heavy chain FR4 as set forth by SEQ ID NO: 308. In some embodiments, an anti-TL1A antibody comprises a light chain FR1 as set forth by SEQ ID NO: 309. In some embodiments, an anti-TL1A antibody comprises a light chain FR2 as set forth by SEQ ID NO: 310. In some embodiments, an anti-TL1A antibody comprises a light chain FR2 as set forth by SEQ ID NO: 1324. In some embodiments, an anti-TL1A antibody comprises a light chain FR3 as set forth by SEQ ID NO: 311. In some embodiments, an anti-TL1A antibody comprises a light chain FR3 as set forth by SEQ ID NO: 1325. In some embodiments, an anti-TL1A antibody comprises a light chain FR4 as set forth by SEQ ID NO: 312.

In certain embodiments, an anti-TL1A antibody comprises the framework regions set forth in any one of the antibodies in Table 16 and Table 17, wherein the framework regions are defined by the Aho or Kabat, Chothia, or IMGT method. In certain embodiments, an anti-TL1A antibody comprises the framework regions set forth in antibody A217. In certain embodiments, an anti-TL1A antibody comprises the framework regions set forth in antibody A220. In certain embodiments, an anti-TL1A antibody comprises the framework regions set forth in antibody A223. In certain embodiments, an anti-TL1A antibody comprises the framework regions set forth in antibody A219. In certain embodiments, an anti-TL1A antibody comprises the framework regions set forth in antibody A221. In certain embodiments, an anti-TL1A antibody comprises the framework regions set forth in antibody A200. In certain embodiments, an anti-TL1A antibody comprises the framework regions set forth in antibody A213. In certain embodiments, an anti-TL1A antibody comprises the framework regions set forth in antibody A212. In certain embodiments, an anti-TL1A antibody comprises the framework regions set forth in antibody A107. In certain embodiments, an anti-TL1A antibody comprises the framework regions set forth in antibody A205. In certain embodiments, an anti-TL1A antibody comprises the framework regions set forth in antibody A211. In certain embodiments, an anti-TL1A antibody comprises the framework regions set forth in antibody A199. In certain embodiments, an anti-TL1A antibody comprises the framework regions set forth in antibody A15. In certain embodiments, an anti-TL1A antibody comprises the framework regions set forth in antibody A30. In certain embodiments, an anti-TL1A antibody comprises the framework regions set forth in antibody A100. In certain embodiments, an anti-TL1A antibody comprises the framework regions set forth in antibody A181. In certain embodiments, an anti-TL1A antibody comprises the framework regions set forth in antibody A129. In certain embodiments, an anti-TL1A antibody comprises the framework regions set forth in antibody A214. In certain embodiments, an anti-TL1A antibody comprises the framework regions set forth in antibody A216. In certain embodiments, an anti-TL1A antibody comprises the framework regions set forth in antibody A122. In certain embodiments, an anti-TL1A antibody comprises the framework regions set forth in antibody A222. In certain embodiments, an anti-TL1A antibody comprises the framework regions set forth in antibody A188. In certain embodiments, an anti-TL1A antibody comprises the framework regions set forth in antibody A203. In certain embodiments, an anti-TL1A antibody comprises the framework regions set forth in antibody A147. In certain embodiments, an anti-TL1A antibody comprises the framework regions set forth in antibody A127. In certain embodiments, an anti-TL1A antibody comprises the framework regions set forth in antibody A126. In certain embodiments, an anti-TL1A antibody comprises the framework regions set forth in antibody A160. In certain embodiments, an anti-TL1A antibody comprises the framework regions set forth in antibody A157. In certain embodiments, an anti-TL1A antibody comprises the framework regions set forth in antibody A159. In certain embodiments, an anti-TL1A antibody comprises the framework regions set forth in antibody A218. In certain embodiments, an anti-TL1A antibody comprises the framework regions set forth in antibody A158. In certain embodiments, an anti-TL1A antibody comprises the framework regions set forth in antibody A125. In certain embodiments, an anti-TL1A antibody comprises the framework regions set forth in antibody A103. In certain embodiments, an anti-TL1A antibody comprises the framework regions set forth in antibody A64. In certain embodiments, an anti-TL1A antibody comprises the framework regions set forth in antibody A67. In certain embodiments, an anti-TL1A antibody comprises the framework regions set forth in antibody A138. In certain embodiments, an anti-TL1A antibody comprises the framework regions set forth in antibody A68. In certain embodiments, an anti-TL1A antibody comprises the framework regions set forth in antibody A94. In certain embodiments, an anti-TL1A antibody comprises the framework regions set forth in antibody A110. In certain embodiments, an anti-TL1A antibody comprises the framework regions set forth in antibody A197. In certain embodiments, an anti-TL1A antibody comprises the framework regions set forth in antibody A112. In certain embodiments, an anti-TL1A antibody comprises the framework regions set forth in antibody A169. In certain embodiments, an anti-TL1A antibody comprises the framework regions set forth in antibody A173. In certain embodiments, an anti-TL1A antibody comprises the framework regions set forth in antibody A179. In certain embodiments, an anti-TL1A antibody comprises the framework regions set forth in antibody A148. In certain embodiments, an anti-TL1A antibody comprises the framework regions set forth in antibody A115. In certain embodiments, an anti-TL1A antibody comprises the framework regions set forth in antibody A149. In certain embodiments, an anti-TL1A antibody comprises the framework regions set forth in antibody A134. In certain embodiments, an anti-TL1A antibody comprises the framework regions set forth in antibody A113. In certain embodiments, an anti-TL1A antibody comprises the framework regions set forth in antibody A151. In certain embodiments, an anti-TL1A antibody comprises the framework regions set forth in antibody A96. In certain embodiments, an anti-TL1A antibody comprises the framework regions set forth in antibody A132. In certain embodiments, an anti-TL1A antibody comprises the framework regions set forth in antibody A196. In certain embodiments, an anti-TL1A antibody comprises the framework regions set forth in antibody A172. In certain embodiments, an anti-TL1A antibody comprises the framework regions set forth in antibody A75. In certain embodiments, an anti-TL1A antibody comprises the framework regions set forth in antibody A174. In certain embodiments, an anti-TL1A antibody comprises the framework regions set forth in antibody A109. In certain embodiments, an anti-TL1A antibody comprises the framework regions set forth in antibody A198. In certain embodiments, an anti-TL1A antibody comprises the framework regions set forth in antibody A170.

In certain embodiments, an anti-TL1A antibody comprises the heavy chain framework regions set forth in any one of the antibodies in Table 16, and the light chain framework regions set forth in any one of the antibodies in Table 17, wherein the framework regions are defined by the Aho or Kabat, Chothia, or IMGT method.

### 5.3.1.3 Exemplary Anti-TL1A Variable Regions

In one aspect, provided herein is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 101-135; and a light chain variable region at least about 85%, 90%, 91%, 92%, 93%, 94%, 95%, 96%, 97%, 98%, 99%, or 100% identical to any one of SEQ ID NOS: 201-206.

Further provided herein is a first embodiment of an anti-TL1A antibody comprising a heavy chain variable region and a light chain variable region. Non-limiting additional embodiments include: (Embodiment 2) The anti-TL1A antibody of embodiment 1, wherein the heavy chain variable region comprises SEQ ID NO: 101. (Embodiment 3) The anti-TL1A antibody of embodiment 1, wherein the heavy chain variable region comprises a sequence at least about 90%, 95%, 96%, 97%, 98%, 99%, or 100% identical to SEQ ID NO: 101. (Embodiment 4) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 101. (Embodiment 5) The anti-TL1A antibody of embodiment 1, wherein the heavy chain variable region comprises SEQ ID NO: 102. (Embodiment 6) The anti-TL1A antibody of embodiment 1, wherein the heavy chain variable region comprises a sequence at least about 90%, 95%, 96%, 97%, 98%, 99%, or 100% identical to SEQ ID NO: 102. (Embodiment 7) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 102. (Embodiment 8) The anti-TL1A antibody of embodiment 1, wherein the heavy chain variable region comprises SEQ ID NO: 103. (Embodiment 9) The anti-TL1A antibody of embodiment 1, wherein the heavy chain variable region comprises a sequence at least about 90%, 95%, 96%, 97%, 98%, 99%, or 100% identical to SEQ ID NO: 103. (Embodiment 10) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 103.

(Embodiment 11) The anti-TL1A antibody of embodiment 1, wherein the heavy chain variable region comprises SEQ ID NO: 104. (Embodiment 12) The anti-TL1A antibody of embodiment 1, wherein the heavy chain variable region comprises a sequence at least about 90%, 95%, 96%, 97%, 98%, 99%, or 100% identical to SEQ ID NO: 104. (Embodiment 13) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 104. (Embodiment 14) The anti-TL1A antibody of embodiment 1, wherein the heavy chain variable region comprises SEQ ID NO: 105. (Embodiment 1S) The anti-TL1A antibody of embodiment 1, wherein the heavy chain variable region comprises a sequence at least about 90%, 95%, 96%, 97%, 98%, 99%, or 100% identical to SEQ ID NO: 105. (Embodiment 16) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 105. (Embodiment 17) The anti-TL1A antibody of embodiment 1, wherein the heavy chain variable region comprises SEQ ID NO: 106. (Embodiment 18) The anti-TL1A antibody of embodiment 1, wherein the heavy chain variable region comprises a sequence at least about 90%, 95%, 96%, 97%, 98%, 99%, or 100% identical to SEQ ID NO: 106. (Embodiment 19) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 106. (Embodiment 20) The anti-TL1A antibody of embodiment 1, wherein the heavy chain variable region comprises SEQ ID NO: 107. (Embodiment 21) The anti-TL1A antibody of embodiment 1, wherein the heavy chain variable region comprises a sequence at least about 90%, 95%, 96%, 97%, 98%, 99%, or 100% identical to SEQ ID NO: 107. (Embodiment 22) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 107.

(Embodiment 23) The anti-TL1A antibody of embodiment 1, wherein the heavy chain variable region comprises SEQ ID NO: 108. (Embodiment 24) The anti-TL1A antibody of embodiment 1, wherein the heavy chain variable region comprises a sequence at least about 90%, 95%, 96%, 97%, 98%, 99%, or 100% identical to SEQ ID NO: 108. (Embodiment 25) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 108. (Embodiment 26) The anti-TL1A antibody of embodiment 1, wherein the heavy chain variable region comprises SEQ ID NO: 109. (Embodiment 27) The anti-TL1A antibody of embodiment 1, wherein the heavy chain variable region comprises a sequence at least about 90%, 95%, 96%, 97%, 98%, 99%, or 100% identical to SEQ ID NO: 109. (Embodiment 28) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 109. (Embodiment 29) The anti-TL1A antibody of embodiment 1, wherein the heavy chain variable region comprises SEQ ID NO: 110. (Embodiment 30) The anti-TL1A antibody of embodiment 1, wherein the heavy chain variable region comprises a sequence at least about 90%, 95%, 96%, 97%, 98%, 99%, or 100% identical to SEQ ID NO: 110. (Embodiment 31) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 110. (Embodiment 32) The anti-TL1A antibody of embodiment 1, wherein the heavy chain variable region comprises SEQ ID NO: 111. (Embodiment 33) The anti-TL1A antibody of embodiment 1, wherein the heavy chain variable region comprises a sequence at least about 90%, 95%, 96%, 97%, 98%, 99%, or 100% identical to SEQ ID NO: 111. (Embodiment 34) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 111. (Embodiment 35) The anti-TL1A antibody of embodiment 1, wherein the heavy chain variable region comprises SEQ ID NO: 112. (Embodiment 36) The anti-TL1A antibody of embodiment 1, wherein the heavy chain variable region comprises a sequence at least about 90%, 95%, 96%, 97%, 98%, 99%, or 100% identical to SEQ ID NO: 112. (Embodiment 37) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 112. (Embodiment 38) The anti-TL1A antibody of embodiment 1, wherein the heavy chain variable region comprises SEQ ID NO: 113. (Embodiment 39) The anti-TL1A antibody of embodiment 1, wherein the heavy chain variable region comprises a sequence at least about 90%, 95%, 96%, 97%, 98%, 99%, or 100% identical to SEQ ID NO: 113. (Embodiment 40) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 113.

(Embodiment 41) The anti-TL1A antibody of embodiment 1, wherein the heavy chain variable region comprises SEQ ID NO: 114. (Embodiment 42) The anti-TL1A antibody of embodiment 1, wherein the heavy chain variable region comprises a sequence at least about 90%, 95%, 96%, 97%, 98%, 99%, or 100% identical to SEQ ID NO: 114. (Embodiment 43) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 114. (Embodiment 44) The anti-TL1A antibody of embodiment 1, wherein the heavy chain variable region comprises SEQ ID NO: 115. (Embodiment 45) The anti-TL1A antibody of embodiment 1, wherein the heavy chain variable region comprises a sequence at least about 90%, 95%, 96%, 97%, 98%, 99%, or 100% identical to SEQ ID NO: 115. (Embodiment 46) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 115. (Embodiment 47) The anti-TL1A antibody of embodiment 1, wherein the heavy chain variable region comprises SEQ ID NO: 116. (Embodiment 48) The anti-TL1A antibody of embodiment 1, wherein the heavy chain variable region comprises a sequence at least about 90%, 95%, 96%, 97%, 98%, 99%, or 100% identical to SEQ ID NO: 116. (Embodiment 49) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 116. (Embodiment 50) The anti-TL1A antibody of embodiment 1, wherein the heavy chain variable region comprises SEQ ID NO: 117. (Embodiment 51) The anti-TL1A antibody of embodiment 1, wherein the heavy chain variable region comprises a sequence at least about 90%, 95%, 96%, 97%, 98%, 99%, or 100% identical to SEQ ID NO: 117. (Embodiment $2) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 117.

(Embodiment 53) The anti-TL1A antibody of embodiment 1, wherein the heavy chain variable region comprises SEQ ID NO: 118. (Embodiment 54) The anti-TL1A antibody of embodiment 1, wherein the heavy chain variable region comprises a sequence at least about 90%, 95%, 96%, 97%, 98%, 99%, or 100% identical to SEQ ID NO: 118. (Embodiment 55) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 118. (Embodiment 56) The anti-TL1A antibody of embodiment 1, wherein the heavy chain variable region comprises SEQ ID NO: 119. (Embodiment 57) The anti-TL1A antibody of embodiment 1, wherein the heavy chain variable region comprises a sequence at least about 90%, 95%, 96%, 97%, 98%, 99%, or 100% identical to SEQ ID NO: 119. (Embodiment 58) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 119. (Embodiment 59) The anti-TL1A antibody of embodiment 1, wherein the heavy chain variable region comprises SEQ ID NO: 120. (Embodiment 60) The anti-TL1A antibody of embodiment 1, wherein the heavy chain variable region comprises a sequence at least about 90%, 95%, 96%, 97%, 98%, 99%, or 100% identical to SEQ ID NO: 120. (Embodiment 61) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 120.

(Embodiment 62) The anti-TL1A antibody of embodiment 1, wherein the heavy chain variable region comprises SEQ ID NO: 121. (Embodiment 63) The anti-TL1A antibody of embodiment 1, wherein the heavy chain variable region comprises a sequence at least about 90%, 95%, 96%, 97%, 98%, 99%, or 100% identical to SEQ ID NO: 121. (Embodiment 64) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 121. (Embodiment 65) The anti-TL1A antibody of embodiment 1, wherein the heavy chain variable region comprises SEQ ID NO: 122. (Embodiment 66) The anti-TL1A antibody of embodiment 1, wherein the heavy chain variable region comprises a sequence at least about 90%, 95%, 96%, 97%, 98%, 99%, or 100% identical to SEQ ID NO: 122. (Embodiment 67) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 122. (Embodiment 68) The anti-TL1A antibody of embodiment 1, wherein the heavy chain variable region comprises SEQ ID NO: 123.

(Embodiment 69) The anti-TL1A antibody of embodiment 1, wherein the heavy chain variable region comprises a sequence at least about 90%, 95%, 96%, 97%, 98%, 99%, or 100% identical to SEQ ID NO: 123. (Embodiment 70) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 123. (Embodiment 71) The anti-TL1A antibody of embodiment 1, wherein the heavy chain variable region comprises SEQ ID NO: 124. (Embodiment 72) The anti-TL1A antibody of embodiment 1, wherein the heavy chain variable region comprises a sequence at least about 90%, 95%, 96%, 97%, 98%, 99%, or 100% identical to SEQ ID NO: 124. (Embodiment 73) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 124.

(Embodiment 74) The anti-TL1A antibody of embodiment 1, wherein the heavy chain variable region comprises SEQ ID NO: 125. (Embodiment 75) The anti-TL1A antibody of embodiment 1, wherein the heavy chain variable region comprises a sequence at least about 90%, 95%, 96%, 97%, 98%, 99%, or 100% identical to SEQ ID NO: 125. (Embodiment 76) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 125. (Embodiment 77) The anti-TL1A antibody of embodiment 1, wherein the heavy chain variable region comprises SEQ ID NO: 126. (Embodiment 78) The anti-TL1A antibody of embodiment 1, wherein the heavy chain variable region comprises a sequence at least about 90%, 95%, 96%, 97%, 98%, 99%, or 100% identical to SEQ ID NO: 126. (Embodiment 79) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 126. (Embodiment 80) The anti-TL1A antibody of embodiment 1, wherein the heavy chain variable region comprises SEQ ID NO: 127. (Embodiment 81) The anti-TL1A antibody of embodiment 1, wherein the heavy chain variable region comprises a sequence at least about 90%, 95%, 96%, 97%, 98%, 99%, or 100% identical to SEQ ID NO: 127. (Embodiment 82) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 127. (Embodiment 83) The anti-TL1A antibody of embodiment 1, wherein the heavy chain variable region comprises SEQ ID NO: 128. (Embodiment 84) The anti-TL1A antibody of embodiment 1, wherein the heavy chain variable region comprises a sequence at least about 90%, 95%, 96%, 97%, 98%, 99%, or 100% identical to SEQ ID NO: 128. (Embodiment 85) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 128.

(Embodiment 86) The anti-TL1A antibody of embodiment 1, wherein the heavy chain variable region comprises SEQ ID NO: 129. (Embodiment 87) The anti-TL1A antibody of embodiment 1, wherein the heavy chain variable region comprises a sequence at least about 90%, 95%, 96%, 97%, 98%, 99%, or 100% identical to SEQ ID NO: 129. (Embodiment 88) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 129. (Embodiment 89) The anti-TL1A antibody of embodiment 1, wherein the heavy chain variable region comprises SEQ ID NO: 130. (Embodiment 90) The anti-TL1A antibody of embodiment 1, wherein the heavy chain variable region comprises a sequence at least about 90%, 95%, 96%, 97%, 98%, 99%, or 100% identical to SEQ ID NO: 130. (Embodiment 91) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 130. (Embodiment 92) The anti-TL1A antibody of embodiment 1, wherein the heavy chain variable region comprises SEQ ID NO: 131. (Embodiment 93) The anti-TL1A antibody of embodiment 1, wherein the heavy chain variable region comprises a sequence at least about 90%, 95%, 96%, 97%, 98%, 99%, or 100% identical to SEQ ID NO: 131. (Embodiment 94) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 131. (Embodiment 95) The anti-TL1A antibody of embodiment 1, wherein the heavy chain variable region comprises SEQ ID NO: 132. (Embodiment 96) The anti-TL1A antibody of embodiment 1, wherein the heavy chain variable region comprises a sequence at least about 90%, 95%, 96%, 97%, 98%, 99%, or 100% identical to SEQ ID NO: 132. (Embodiment 97) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 132.

(Embodiment 98) The anti-TL1A antibody of embodiment 1, wherein the heavy chain variable region comprises SEQ ID NO: 133. (Embodiment 99) The anti-TL1A antibody of embodiment 1, wherein the heavy chain variable region comprises a sequence at least about 90%, 95%, 96%, 97%, 98%, 99%, or 100% identical to SEQ ID NO: 133. (Embodiment 100) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 133. (Embodiment 101) The anti-TL1A antibody of embodiment 1, wherein the heavy chain variable region comprises SEQ ID NO: 134. (Embodiment 102) The anti-TL1A antibody of embodiment 1, wherein the heavy chain variable region comprises a sequence at least about 90%, 95%, 96%, 97%, 98%, 99%, or 100% identical to SEQ ID NO: 134. (Embodiment 103) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 134. (Embodiment 104) The anti-TL1A antibody of embodiment 1, wherein the heavy chain variable region comprises SEQ ID NO: 135. (Embodiment 105) The anti-TL1A antibody of embodiment 1, wherein the heavy chain variable region comprises a sequence at least about 90%, 95%, 96%, 97%, 98%, 99%, or 100% identical to SEQ ID NO: 135. (Embodiment 106) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 135.

(Embodiment 107) The anti-TL1A antibody of any one of embodiments 1-106, wherein the light chain variable region comprises SEQ ID NO: 201. (Embodiment 108) The anti-TL1A antibody of any one of embodiments 1-106, wherein the light chain variable region comprises a sequence at least about 90%, 95%, 96%, 97%, 98%, 99%, or 100% identical to SEQ ID NO: 201. (Embodiment 109) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 201. (Embodiment 110) The anti-TL1A antibody of any one of embodiments 1-106, wherein the light chain variable region comprises SEQ ID NO: 202. (Embodiment 111) The anti-TL1A antibody of any one of embodiments 1-106, wherein the light chain variable region comprises a sequence at least about 90%, 95%, 96%, 97%, 98%, 99%, or 100% identical to SEQ ID NO: 202. (Embodiment 112) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 202. (Embodiment 113) The anti-TL1A antibody of any one of embodiments 1-106, wherein the light chain variable region comprises SEQ ID NO: 203. (Embodiment 114) The anti-TL1A antibody of any one of embodiments 1~106, wherein the light chain variable region comprises a sequence at least about 90%, 95%, 96%, 97%, 98%, 99%, or 100% identical to SEQ ID NO: 203. (Embodiment 115) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 203. (Embodiment 116) The anti-TL1A antibody of any one of embodiments 1-106, wherein the light chain variable region comprises SEQ ID NO: 204. (Embodiment 117) The anti-TL1A antibody of any one of embodiments 1-106, wherein the light chain variable region comprises a sequence at least about 90%, 95%, 96%, 97%, 98%, 99%, or 100% identical to SEQ ID NO: 204. (Embodiment 118) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 204. (Embodiment 119) The anti-TL1A antibody of any one of embodiments 1-106, wherein the light chain variable region comprises SEQ ID NO: 205. (Embodiment 120) The anti-TL1A antibody of any one of embodiments 1-106, wherein the light chain variable region comprises a sequence at least about 90%, 95%, 96%, 97%, 98%, 99%, or 100% identical to SEQ ID NO: 205. (Embodiment 121) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 205. (Embodiment 122) The anti-TL1A antibody of any one of embodiments 1-106, wherein the light chain variable region comprises SEQ ID NO: 206. (Embodiment 123) The anti-TL1A antibody of any one of embodiments 1-106, wherein the light chain variable region comprises a sequence at least about 90%, 95%, 96%, 97%, 98%, 99%, or 100% identical to SEQ ID NO: 206. (Embodiment 124) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 206.

(Embodiment 125) The anti-TL1A antibody of embodiment 1, comprising A217. (Embodiment 126) The anti-TL1A antibody of embodiment 1, wherein the heavy chain variable region comprises a sequence at least about 90%, 95%, 96%, 97%, 98%, 99%, or 100% identical to SEQ ID NO: 101, and the light chain variable region comprises a sequence at least about 90%, 95%, 96%, 97%, 98%, 99%, or 100% identical to SEQ ID NO: 201. (Embodiment 127) The anti-TL1A antibody of embodiment 1, comprising A220. (Embodiment 128) The anti-TL1A antibody of embodiment 1, wherein the heavy chain variable region comprises a sequence at least about 90%, 95%, 96%, 97%, 98%, 99%, or 100% identical to SEQ ID NO: 102, and the light chain variable region comprises a sequence at least about 90%, 95%, 96%, 97%, 98%, 99%, or 100% identical to SEQ ID NO: 201. (Embodiment 129) The anti-TL1A antibody of embodiment 1, comprising A223. (Embodiment 130) The anti-TL1A antibody of embodiment 1, wherein the heavy chain variable region comprises a sequence at least about 90%, 95%, 96%, 97%, 98%, 99%, or 100% identical to SEQ ID NO: 103, and the light chain variable region comprises a sequence at least about 90%, 95%, 96%, 97%, 98%, 99%, or 100% identical to SEQ ID NO: 202. (Embodiment 131) The anti-TL1A antibody of embodiment 1, comprising A219. (Embodiment 132) The anti-TL1A antibody of embodiment 1, wherein the heavy chain variable region comprises a sequence at least about 90%, 95%, 96%, 97%, 98%, 99%, or 100% identical to SEQ ID NO: 104, and the light chain variable region comprises a sequence at least about 90%, 95%, 96%, 97%, 98%, 99%, or 100% identical to SEQ ID NO: 201. (Embodiment 133) The anti-TL1A antibody of embodiment 1, comprising A221. (Embodiment 134) The anti-TL1A antibody of embodiment 1, wherein the heavy chain variable region comprises a sequence at least about 90%, 95%, 96%, 97%, 98%, 99%, or 100% identical to SEQ ID NO: 105, and the light chain variable region comprises a sequence at least about 90%, 95%, 96%, 97%, 98%, 99%, or 100% identical to SEQ ID NO: 201.

(Embodiment 135) The anti-TL1A antibody of embodiment 1, comprising A200. (Embodiment 136) The anti-TL1A antibody of embodiment 1, wherein the heavy chain variable region comprises a sequence at least about 90%, 95%, 96%, 97%, 98%, 99%, or 100% identical to SEQ ID NO: 103, and the light chain variable region comprises a sequence at least about 90%, 95%, 96%, 97%, 98%, 99%, or 100% identical to SEQ ID NO: 201. (Embodiment 137) The anti-TL1A antibody of embodiment 1, comprising A213. (Embodiment 138) The anti-TL1A antibody of embodiment 1, wherein the heavy chain variable region comprises a sequence at least about 90%, 95%, 96%, 97%, 98%, 99%, or 100% identical to SEQ ID NO: 106, and the light chain variable region comprises a sequence at least about 90%, 95%, 96%, 97%, 98%, 99%, or 100% identical to SEQ ID NO: 201. (Embodiment 139) The anti-TL1A antibody of embodiment 1, comprising A212. (Embodiment 140) The anti-TL1A antibody of embodiment 1, wherein the heavy chain variable region comprises a sequence at least about 90%, 95%, 96%, 97%, 98%, 99%, or 100% identical to SEQ ID NO: 107, and the light chain variable region comprises a sequence at least about 90%, 95%, 96%, 97%, 98%, 99%, or 100% identical to SEQ ID NO: 202. (Embodiment 141) The anti-TL1A antibody of embodiment 1, comprising A107. (Embodiment 142) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 202. (Embodiment 143) The anti-TL1A antibody of embodiment 1, comprising A205. (Embodiment 144) The anti-TL1A antibody of embodiment 1, wherein the heavy chain variable region comprises a sequence at least about 90%, 95%, 96%, 97%, 98%, 99%, or 100% identical to SEQ ID NO: 109, and the light chain variable to SEQ ID NO: 202.

(Embodiment 145) The anti-TL1A antibody of embodiment 1, comprising A211. (Embodiment 146) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 201. (Embodiment 147) The anti-TL1A antibody of embodiment 1, comprising A199. (Embodiment 148) The anti-TL1A antibody of embodiment 1, wherein the heavy chain variable region comprises a sequence at least about 90%, 95%, 96%, 97%, 98%, 99%, or 100% identical to SEQ ID NO: 109, and the light chain variable region comprises a sequence at least about 90%, 95%, 96%, 97%, 98%, 99%, or 100% identical to SEQ ID NO: 201. (Embodiment 149) The anti-TL1A antibody of embodiment 1, comprising A15. (Embodiment 150) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 203. (Embodiment 151) The anti-TL1A antibody of embodiment 1, comprising A30. (Embodiment 152) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 204. (Embodiment 153) The anti-TL1A antibody of embodiment 1, comprising A100. (Embodiment 154) The anti-TL1A antibody of embodiment 1, wherein the heavy chain variable region comprises a sequence at least about 90%, 95%, 96%, 97%, 98%, 99%, or 100% identical to SEQ ID NO: 107, and the light chain variable region comprises a sequence at least about 90%, 95%, 96%, 97%, 98%, 99%, or 100% identical to SEQ ID NO: 204.

(Embodiment 155) The anti-TL1A antibody of embodiment 1, comprising A181. (Embodiment 156) The anti-TL1A antibody of embodiment 1, wherein the heavy chain variable region comprises a sequence at least about 90%, 95%, 96%, 97%, 98%, 99%, or 100% identical to SEQ ID NO: 107, and the light chain variable region comprises a sequence at least about 90%, 95%, 96%, 97%, 98%, 99%, or 100% identical to SEQ ID NO: 202. (Embodiment 157) The anti-TL1A antibody of embodiment 1, comprising A129. (Embodiment 158) The anti-TL1A antibody 90%, 95%, 96%, 97%, 98%, 99%, or 100% identical to SEQ ID NO: 110, and the light chain variable region comprises a sequence at least about 90%, 95%, 96%, 97%, 98%, 99%, or 100% identical to SEQ ID NO: 204. (Embodiment 159) The anti-TL1A antibody of embodiment 1, comprising A214. (Embodiment 160) The anti-TL1A antibody of embodiment 1, wherein the heavy chain variable region comprises a sequence at least about 90%, 95%, 96%, 97%, 98%, 99%, or 100% identical to SEQ ID NO: 111, and the light chain variable region comprises a sequence at least about 90%, 95%, 96%, 97%, 98%, 99%, or 100% identical to SEQ ID NO: 201. (Embodiment 161) The anti-TL1A antibody of embodiment 1, comprising A216. (Embodiment 162) The anti-TL1A antibody of embodiment 1, wherein the heavy chain variable region comprises a sequence at least about 90%, 95%, 96%, 97%, 98%, 99%, or 100% identical to SEQ ID NO: 112, and the light chain variable region comprises a sequence at least about 90%, 95%, 96%, 97%, 98%, 99%, or 100% identical to SEQ ID NO: 201. (Embodiment 163) The anti-TL1A antibody of embodiment 1, comprising A122. (Embodiment 164) The anti-TL1A antibody of embodiment 1, wherein the heavy chain variable region comprises a sequence at least about 90%, 95%, 96%, 97%, 98%, 99%, or 100% identical to SEQ ID NO: 113, and the light chain variable region comprises a sequence at least about 90%, 95%, 96%, 97%, 98%, 99%, or 100% identical to SEQ ID NO: 204.

(Embodiment 165) The anti-TL1A antibody of embodiment 1, comprising A222. (Embodiment 166) The anti-TL1A antibody of embodiment 1, wherein the heavy chain variable region comprises a sequence at least about 90%, 95%, 96%, 97%, 98%, 99%, or 100% identical to SEQ ID NO: 114, and the light chain variable region comprises a sequence at least about 90%, 95%, 96%, 97%, 98%, 99%, or 100% identical to SEQ ID NO: 201. (Embodiment 167) The anti-TL1A antibody of embodiment 1, comprising A188. (Embodiment 168) The anti-TL1A antibody of embodiment 1, wherein the heavy chain variable region comprises a sequence at least about 90%, 95%, 96%, 97%, 98%, 99%, or 100% identical to SEQ ID NO: 115, and the light chain variable region comprises a sequence at least about 90%, 95%, 96%, 97%, 98%, 99%, or 100% identical to SEQ ID NO: 202. (Embodiment 169) The anti-TL1A antibody of embodiment 1, comprising A203. (Embodiment 170) The anti-TL1A antibody of embodiment 1, wherein the heavy chain variable region comprises a sequence at least about 90%, 95%, 96%, 97%, 98%, 99%, or 100% identical to SEQ ID NO: 116, and the light chain variable region comprises a sequence at least about 90%, 95%, 96%, 97%, 98%, 99%, or 100% identical to SEQ ID NO: 201. (Embodiment 171) The anti-TL1A antibody of embodiment 1, comprising A147. (Embodiment 172) The anti-TL1A antibody of embodiment 1, wherein the heavy chain variable region comprises a sequence at least about 90%, 95%, 96%, 97%, 98%, 99%, or 100% identical to SEQ ID NO: 117, and the light chain variable region comprises a sequence at least about 90%, 95%, 96%, 97%, 98%, 99%, or 100% identical to SEQ ID NO: 201. (Embodiment 173) The anti-TL1A antibody of embodiment 1, comprising A127. (Embodiment 174) The anti-TL1A antibody of embodiment 1, wherein the heavy chain variable region comprises a sequence at least about 90%, 95%, 96%, 97%, 98%, 99%, or 100% identical to SEQ ID NO: 118, and the light chain variable region comprises a sequence at least about 90%, 95%, 96%, 97%, 98%, 99%, or 100% identical to SEQ ID NO: 204.

(Embodiment 175) The anti-TL1A antibody of embodiment 1, comprising A126. (Embodiment 176) The anti-TL1A antibody of embodiment 1, wherein the heavy chain variable region comprises a sequence at least about 90%, 95%, 96%, 97%, 98%, 99%, or 100% identical to SEQ ID NO: 114, and the light chain variable region comprises a sequence at least about 90%, 95%, 96%, 97%, 98%, 99%, or 100% identical to SEQ ID NO: 204. (Embodiment 177) The anti-TL1A antibody of embodiment 1, comprising A160. (Embodiment 178) The anti-TL1A antibody of embodiment 1, wherein the heavy chain variable region comprises a sequence at least about 90%, 95%, 96%, 97%, 98%, 99%, or 100% identical to SEQ ID NO: 102, and the light chain variable region comprises a sequence at least about 90%, 95%, 96%, 97%, 98%, 99%, or 100% identical to SEQ ID NO: 204. (Embodiment 179) The anti-TL1A antibody of embodiment 1, comprising A157. (Embodiment 180) The anti-TL1A antibody of embodiment 1, wherein the heavy chain variable region comprises a sequence at least about 90%, 95%, 96%, 97%, 98%, 99%, or 100% identical to SEQ ID NO: 104, and the light chain variable region comprises a sequence at least about 90%, 95%, 96%, 97%, 98%, 99%, or 100% identical to SEQ ID NO: 204. (Embodiment 181) The anti-TL1A antibody of embodiment 1, comprising A159. (Embodiment 182) The anti-TL1A antibody of embodiment 1, wherein the heavy chain variable region comprises a sequence at least about 90%, 95%, 96%, 97%, 98%, 99%, or 100% identical to SEQ ID NO: 119, and the light chain variable region comprises a sequence at least about 90%, 95%, 96%, 97%, 98%, 99%, or 100% identical to SEQ ID NO: 204. (Embodiment 183) The anti-TL1A antibody of embodiment 1, comprising A218. (Embodiment 184) The anti-TL1A antibody of embodiment 1, wherein the heavy chain variable region comprises a sequence at least about 90%, 95%, 96%, 97%, 98%, 99%, or 100% identical to SEQ ID NO: 119, and the light chain variable to SEQ ID NO: 201.

(Embodiment 185) The anti-TL1A antibody of embodiment 1, comprising A158. (Embodiment 186) The anti-TL1A antibody of embodiment 1, wherein the heavy chain variable region comprises a sequence at least about 90%, 95%, 96%, 97%, 98%, 99%, or 100% identical to SEQ ID NO: 101, and the light chain variable region comprises a sequence at least about 90%, 95%, 96%, 97%, 98%, 99%, or 100% identical to SEQ ID NO: 204. (Embodiment 187) The anti-TL1A antibody of embodiment 1, comprising A125, (Embodiment 188) The anti-TL1A antibody of embodiment 1, wherein the heavy chain variable region comprises a sequence at least about 90%, 95%, 96%, 97%, 98%, 99%, or 100% identical to SEQ ID NO: 105, and the light chain variable region comprises a sequence at least about 90%, 95%, 96%, 97%, 98%, 99%, or 100% identical to SEQ ID NO: 204. (Embodiment 189) The anti-TL1A antibody of embodiment 1, comprising A103. (Embodiment 190) The anti-TL1A antibody of embodiment 1, wherein the heavy chain variable region comprises a sequence at least about 90%, 95%, 96%, 97%, 98%, 99%, or 100% identical to SEQ ID NO: 120, and the light chain variable region comprises a sequence at least about 90%, 95%, 96%, 97%, 98%, 99%, or 100% identical to SEQ ID NO: 204. (Embodiment 191) The anti-TL1A antibody of embodiment 1, comprising A64. (Embodiment 192) The anti-TL1A antibody of embodiment 1, wherein the heavy chain variable region comprises a sequence at least about 90%, 95%, 96%, 97%, 98%, 99%, or 100% identical to SEQ ID NO: 121, and the light chain variable region comprises a sequence at least about 90%, 95%, 96%, 97%, 98%, 99%, or 100% identical to SEQ ID NO: 202. (Embodiment 193) The anti-TL1A antibody of embodiment 1, comprising A67. (Embodiment 194) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 202.

(Embodiment 195) The anti-TL1A antibody of embodiment 1 comprising A138. (Embodiment 196) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 204. (Embodiment 197) The anti-TL1A antibody of embodiment 1, comprising A68. (Embodiment 198) The anti-TL1A antibody 90%, 95%, 96%, 97%, 98%, 99%, or 100% identical to SEQ ID NO: 123, and the light chain variable region comprises a sequence at least about 90%, 95%, 96%, 97%, 98%, 99%, or 100% identical to SEQ ID NO: 202. (Embodiment 199) The anti-TL1A antibody of embodiment 1, comprising A94. (Embodiment 200) The anti-TL1A antibody of embodiment 1, wherein the heavy chain variable region comprises a sequence at least about 90%, 95%, 96%, 97%, 98%, 99%, or 100% identical to SEQ ID NO: 124, and the light chain variable region comprises a sequence at least about 90%, 95%, 96%, 97%, 98%, 99%, or 100% identical to SEQ ID NO: 202. (Embodiment 201) The anti-TL1A antibody of embodiment 1, comprising A110. (Embodiment 202) The anti-TL1A antibody of embodiment 1, wherein the heavy chain variable region comprises a sequence at least about 90%, 95%, 96%, 97%, 98%, 99%, or 100% identical to SEQ ID NO: 125, and the light chain variable region comprises a sequence at least about 90%, 95%, 96%, 97%, 98%, 99%, or 100% identical to SEQ ID NO: 205. (Embodiment 203) The anti-TL1A antibody of embodiment 1, comprising A197. (Embodiment 204) The anti-TL1A antibody of embodiment 1, wherein the heavy chain variable region comprises a sequence at least about 90%, 95%, 96%, 97%, 98%, 99%, or 100% identical to SEQ ID NO: 116, and the light chain variable region comprises a sequence at least about 90%, 95%, 96%, 97%, 98%, 99%, or 100% identical to SEQ ID NO: 205.

(Embodiment 205) The anti-TL1A antibody of embodiment 1, comprising A112. (Embodiment 206) The anti-TL1A antibody of embodiment 1, wherein the heavy chain variable region comprises a sequence at least about 90%, 95%, 96%, 97%, 98%, 99%, or 100% identical to SEQ ID NO: 117, and the light chain variable region comprises a sequence at least about 90%, 95%, 96%, 97%, 98%, 99%, or 100% identical to SEQ ID NO: 205. (Embodiment 207) The anti-TL1A antibody of embodiment 1, comprising A169. (Embodiment 208) The anti-TL1A antibody of embodiment 1, wherein the heavy chain variable region comprises a sequence at least about 90%, 95%, 96%, 97%, 98%, 99%, or 100% identical to SEQ ID NO: 126, and the light chain variable region comprises a sequence at least about 90%, 95%, 96%, 97%, 98%, 99%, or 100% identical to SEQ ID NO: 205. (Embodiment 209) The anti-TL1A antibody of embodiment 1, comprising A173. (Embodiment 210) The anti-TL1A antibody of embodiment 1, wherein the heavy chain variable region comprises a sequence at least about 90%, 95%, 96%, 97%, 98%, 99%, or 100% identical to SEQ ID NO: 127, and the light chain variable region comprises a sequence at least about 90%, 95%, 96%, 97%, 98%, 99%, or 100% identical to SEQ ID NO: 205. (Embodiment 211) The anti-TL1A antibody of embodiment 1, comprising A179. (Embodiment 212) The anti-TL1A antibody of embodiment 1, wherein the heavy chain variable region comprises a sequence at least about 90%, 95%, 96%, 97%, 98%, 99%, or 100% identical to SEQ ID NO: 127, and the light chain variable region comprises a sequence at least about 90%, 95%, 96%, 97%, 98%, 99%, or 100% identical to SEQ ID NO: 201. (Embodiment 213) The anti-TL1A antibody of embodiment 1, comprising A148. (Embodiment 214) The anti-TL1A antibody of embodiment 1, wherein the heavy chain variable region comprises a sequence at least about 90%, 95%, 96%, 97%, 98%, 99%, or 100% identical to SEQ ID NO: 121, and the light chain variable region comprises a sequence at least about 90%, 95%, 96%, 97%, 98%, 99%, or 100% identical to SEQ ID NO: 201.

(Embodiment 215) The anti-TL1A antibody of embodiment 1, comprising A115. (Embodiment 216) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 205. (Embodiment 217) The anti-TL1A antibody of embodiment 1, comprising A149. (Embodiment 218) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 201. (Embodiment 219) The anti-TL1A antibody of embodiment 1, comprising A134. (Embodiment 220) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 206. (Embodiment 221) The anti-TL1A antibody of embodiment 1, comprising Al13. (Embodiment 222) The anti-TL1A antibody of embodiment 1, wherein the heavy chain variable region comprises a sequence at least about 90%, 95%, 96%, 97%, 98%, 99%, or 100% identical to SEQ ID NO: 124, and the light chain variable region comprises a sequence at least about 90%, 95%, 96%, 97%, 98%, 99%, or 100% identical to SEQ ID NO: 205. (Embodiment 223) The anti-TL1A antibody of embodiment 1, comprising A151. (Embodiment 224) The anti-TL1A antibody of embodiment 1, wherein the heavy chain variable region comprises a sequence at least about 90%, 95%, 96%, 97%, 98%, 99%, or 100% identical to SEQ ID NO: 124, and the light chain variable to SEQ ID NO: 201.

(Embodiment 225) The anti-TL1A antibody of embodiment 1, comprising A96. (Embodiment 226) The anti-TL1A antibody of embodiment 1, wherein the heavy chain variable region comprises a sequence at least about 90%, 95%, 96%, 97%, 98%, 99%, or 100% identical to SEQ ID NO: 128, and the light chain variable region comprises a sequence at least about 90%, 95%, 96%, 97%, 98%, 99%, or 100% identical to SEQ ID NO: 205. (Embodiment 227) The anti-TL1A antibody of embodiment 1, comprising A132. (Embodiment 228) The anti-TL1A antibody of embodiment 1, wherein the heavy chain variable region comprises a sequence at least about 90%, 95%, 96%, 97%, 98%, 99%, or 100% identical to SEQ ID NO: 128, and the light chain variable region comprises a sequence at least about 90%, 95%, 96%, 97%, 98%, 99%, or 100% identical to SEQ ID NO: 206. (Embodiment 229) The anti-TL1A antibody of embodiment 1, comprising A196. (Embodiment 230) The anti-TL1A antibody of embodiment 1, wherein the heavy chain variable region comprises a sequence at least about 90%, 95%, 96%, 97%, 98%, 99%, or 100% identical to SEQ ID NO: 129, and the light chain variable region comprises a sequence at least about 90%, 95%, 96%, 97%, 98%, 99%, or 100% identical to SEQ ID NO: 205. (Embodiment 231) The anti-TL1A antibody of embodiment 1, comprising A172. (Embodiment 232) The anti-TL1A antibody of embodiment 1, wherein the heavy chain variable region comprises a sequence at least about 90%, 95%, 96%, 97%, 98%, 99%, or 100% identical to SEQ ID NO: 130, and the light chain variable region comprises a sequence at least about 90%, 95%, 96%, 97%, 98%, 99%, or 100% identical to SEQ ID NO: 205. (Embodiment 233) The anti-TL1A antibody of embodiment 1, comprising A75. (Embodiment 234) The anti-TL1A antibody of embodiment 1, wherein the heavy chain variable region comprises a sequence at least about 90%, 95%, 96%, 97%, 98%, 99%, or 100% identical to SEQ ID NO: 131, and the light chain variable region comprises a sequence at least about 90%, 95%, 96%, 97%, 98%, 99%, or 100% identical to SEQ ID NO: 205.

(Embodiment 235) The anti-TL1A antibody of embodiment 1 comprising A174. (Embodiment 236) The anti-TL1A antibody of embodiment 1, wherein the heavy chain variable region comprises a sequence at least about 90%, 95%, 96%, 97%, 98%, 99%, or 100% identical to SEQ ID NO: 132, and the light chain variable region comprises a sequence at least about 90%, 95%, 96%, 97%, 98%, 99%, or 100% identical to SEQ ID NO: 205. (Embodiment 237) The anti-TL1A antibody of embodiment 1, comprising A109. (Embodiment 238) The anti-TL1A antibody 90%, 95%, 96%, 97%, 98%, 99%, or 100% identical to SEQ ID NO: 133, and the light chain variable region comprises a sequence at least about 90%, 95%, 96%, 97%, 98%, 99%, or 100% identical to SEQ ID NO: 205. (Embodiment 239) The anti-TL1A antibody of embodiment 1, comprising A198. (Embodiment 240) The anti-TL1A antibody of embodiment 1, wherein the heavy chain variable region comprises a sequence at least about 90%, 95%, 96%, 97%, 98%, 99%, or 100% identical to SEQ ID NO: 134, and the light chain variable region comprises a sequence at least about 90%, 95%, 96%, 97%, 98%, 99%, or 100% identical to SEQ ID NO: 205. (Embodiment 241) The anti-TL1A antibody of embodiment 1, comprising A170. (Embodiment 242) The anti-TL1A antibody of embodiment 1, wherein the heavy chain variable region comprises a sequence at least about 90%, 95%, 96%, 97%, 98%, 99%, or 100% identical to SEQ ID NO: 135, and the light chain variable region comprises a sequence at least about 90%, 95%, 96%, 97%, 98%, 99%, or 100% identical to SEQ ID NO: 205. (Embodiment 243) The anti-TL1A antibody of embodiment 1, comprising A500. (Embodiment 244) The anti-TL1A antibody of embodiment 1, comprising A501.

(Embodiment 245) The anti-TL1A of any one of embodiments 1-244, comprising a human IgG1 Fc region comprising: (a) 297A, 297Q, 297G, or 297D, (b) 279F, 279K, or 279L. (c) 228P. (d) 235A, 235E, 235G, 235Q, 235R, or 235S, (e) 237A, 237E, 237K, 237N, or 237R, (f) 234A, 234V, or 234F, (g) 233P. (h) 328A, (i) 327Q or 327T, (j) 329A, 329G, 329Y, or 329R (k) 331S. (1) 236F or 236R, (m) 238A, 238E, 238G, 238H, 238I, 238V, 238 W, or 238Y, (n) 248A, (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P, (nn) 385P, (00) 424H, 424M, or 424V. (pp) 4341. (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L23SE, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (ii) D265A and N297G, (kkk) D270A. (III) A330L, (mmm) P331A or P331S, or (nnn) any combination of (a)-(uu), per Kabat numbering. (Embodiment 246) The anti-TL1A of any one of embodiments 1-244, comprising a (i) human IgG4 Fc region or (ii) a human IgG4 Fc region comprising (a) S228P and L235E, or (b) S228P, F234A, and L235A, per Kabat numbering. (Embodiment 247) The anti-TL1A of any one of embodiments 1-244, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG20). (Embodiment 248) The anti-TL1A of any one of embodiments 1-247, comprising a heavy chain Fc region comprising any one of SEQ ID NOS: 320-362. (Embodiment 249) The anti-TL1A of any one of embodiments 1-248, comprising a light chain constant region comprising SEQ ID NO: 319.

(Embodiment 250) The anti-TL1A of any one of embodiments 1-249, comprising at least about 80% monomeric fraction as determined by the size exclusion chromatography method described herein. (Embodiment 251) The anti-TL1A of any one of embodiments 1-250, comprising at least about 81%, at least about 82%, at least about 83%, or at least about 84% monomeric fraction as determined by the size exclusion chromatography method described herein. (Embodiment 252) The anti-TL1A of any one of embodiments 1-251, comprising at least about 85% monomeric fraction as determined by the size exclusion chromatography method described herein. (Embodiment 253) The anti-TL1A of any one of embodiments 1-252 comprising at least about 86%, at least about 87%, at least about 88%, or at least about 89% monomeric fraction as determined by the size exclusion chromatography method described herein. (Embodiment 254) The anti-TL1A of any one of embodiments 1-253, comprising at least about 90% monomeric fraction as determined by the size exclusion chromatography method described herein. (Embodiment 255) The anti-TL1A of any one of embodiments 1-254, comprising at least about 91%, at least about 92%, at least about 93%, or at least about 94% monomeric fraction as determined by the size exclusion chromatography method described herein. (Embodiment 256) The anti-TL1A of any one of embodiments 1-255, comprising at least about 95% monomeric fraction as determined by the size exclusion chromatography method described herein. (Embodiment 257) The anti-TL1A of any one of embodiments 1-256, comprising at least about 96%, at least about 97%, at least about 98%, or at least about 99% monomeric fraction as determined by the size exclusion chromatography method described herein.

(Embodiment 258) The anti-TL1A of any one of embodiments 1-257, comprising at least about 2 μg/mL expression as determined by the method disclosed herein. (Embodiment 259) The anti-TL1A of any one of embodiments 1-258, comprising between about 2 μg/mL and about 60 μg/mL expression as determined by the method disclosed herein. (Embodiment 260) The anti-TL1A of any one of embodiments 1-259, comprising between about 5 μg/mL and about 60 g/mL expression as determined by the method disclosed herein. (Embodiment 261) The anti-TL1A of any one of embodiments 1-260, comprising between about 10 μg/mL and about 60 μg/mL expression as determined by the method disclosed herein. (Embodiment 262) The anti-TL1A of any one of embodiments 1-261, comprising at least about 5 μg/mL expression as determined by the method disclosed herein. (Embodiment 263) The anti-TL1A of any one of embodiments 1-262, comprising at least about 10 μg/mL expression as determined by the method disclosed herein. (Embodiment 264) The anti-TL1A of any one of embodiments 1-263, comprising at least about 15 μg/mL expression as determined by the method disclosed herein. (Embodiment 265) The anti-TL1A of any one of embodiments 1-264, comprising at least about 20 μg/mL expression as determined by the method disclosed herein.

In one aspect, provided herein is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1200-1263; and a light chain variable region at least about 85%, 90%, 91%, 92%, 93%, 94%, 95%, 96%, 97%, 98%, 99%, or 100% identical to any one of SEQ ID NOS: 1264-1300, 1304-1316.

In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 136; and a light chain variable region at least about 85%, 90%, 91%, 92%, 93%, 94%, 95%, 96%, 97%, 98%, 99%, or 100% identical to SEQ ID NO: 202 (clone 34). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1200; and a light chain variable region at least about 85%, 90%, 91%, 92%, 93%, 94%, 95%, 96%, 97%, 98%, 99%, or 100% identical to SEQ ID NO: 12646 (5C3D11). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1201; and a light chain variable region at least about 85%, 90%, 91%, 92%, 93%, 94%, 95%, 96%, 97%, 98%, 99%, or 100% identical to SEQ ID NO: 1265 (9E12E5). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1202; and a light chain variable region at least about 85%, 90%, 91%, 92%, 93%, 94%, 95%, 96%, 97%, 98%, 99%, or 100% identical to SEQ ID NO: 208 (AS12824). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1203; and a light chain variable region at least about 85%, 90%, 91%, 92%, 93%, 94%, 95%, 96%, 97%, 98%, 99%, or 100% identical to SEQ ID NO: 1266 (AS12823). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1204; and a light chain variable region at least about 85%, 90%, 91%, 92%, 93%, 94%, 95%, 96%, 97%, 98%, 99%, or 100% identical to SEQ ID NO: 1267 (AS12819). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1205; and a light chain variable region at least about 85%, 90%, 91%, 92%, 93%, 94%, 95%, 96%, 97%, 98%, 99%, or 100% identical to SEQ ID NO: 1268 (AS12816). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1206; and a light chain variable region at least about 85%, 90%, 91%, 92%, 93%, 94%, 95%, 96%, 97%, 98%, 99%, or 100% identical to SEQ ID NO: 1269 (AS12825). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1207; and a light chain variable region at least about 85%, 90%, 91%, 92%, 93%, 94%, 95%, 96%, 97%, 98%, 99%, or 100% identical to SEQ ID NO: 1270 (12835). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 202 (18-7 S93E). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 203 (18-7). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1272 (18-7 S92D). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1273 (18-7 S92H). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1274 (18-7 S92N). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1275 (18-7 S92Q). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1276 (18-7 CDRv). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1209; and a light chain variable region at least about 85%, 90%, 91%, 92%, 93%, 94%, 95%, 96%, 97%, 98%, 99%, or 100% identical to SEQ ID NO: 1271 (21-3). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1210; and a light chain variable region at least about 85%, 90%, 91%, 92%, 93%, 94%, 95%, 96%, 97%, 98%, 99%, or 100% identical to SEQ ID NO: 1271 (21-3 V102K). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1211; and a light chain variable region at least about 85%, 90%, 91%, 92%, 93%, 94%, 95%, 96%, 97%, 98%, 99%, or 100% identical to SEQ ID NO: 1271 (21-3 V102M). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1212; and a light chain variable region at least about 85%, 90%, 91%, 92%, 93%, 94%, 95%, 96%, 97%, 98%, 99%, or 100% identical to SEQ ID NO: 1271 (21-3 V102Q). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1213; and a light chain variable region at least about 85%, 90%, 91%, 92%, 93%, 94%, 95%, 96%, 97%, 98%, 99%, or 100% identical to SEQ ID NO: 1271 (21-3 V102 W). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1214; and a light chain variable region at least about 85%, 90%, 91%, 92%, 93%, 94%, 95%, 96%, 97%, 98%, 99%, or 100% identical to SEQ ID NO: 1271 (21-3 CDRv). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1215; and a light chain variable region at least about 85%, 90%, 91%, 92%, 93%, 94%, 95%, 96%, 97%, 98%, 99%, or 100% identical to SEQ ID NO: 1271 (21-3 CDRv). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1216; and a light chain variable region at least about 85%, 90%, 91%, 92%, 93%, 94%, 95%, 96%, 97%, 98%, 99%, or 100% identical to SEQ ID NO: 202 (clone 2). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1216; and a light chain variable region at least about 85%, 90%, 91%, 92%, 93%, 94%, 95%, 96%, 97%, 98%, 99%, or 100% identical to SEQ ID NO: 1277 (clone 52). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1217; and a light chain variable region at least about 85%, 90%, 91%, 92%, 93%, 94%, 95%, 96%, 97%, 98%, 99%, or 100% identical to SEQ ID NO: 202 (clone 46). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1218; and a light chain variable region at least about 85%, 90%, 91%, 92%, 93%, 94%, 95%, 96%, 97%, 98%, 99%, or 100% identical to SEQ ID NO: 202 (clone 47). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1219; and a light chain variable region at least about 85%, 90%, 91%, 92%, 93%, 94%, 95%, 96%, 97%, 98%, 99%, or 100% identical to SEQ ID NO: 1275 (clone 14). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1220; and a light chain variable region at least about 85%, 90%, 91%, 92%, 93%, 94%, 95%, 96%, 97%, 98%, 99%, or 100% identical to SEQ ID NO: 1275 (clone 16L). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1221; and a light chain variable region at least about 85%, 90%, 91%, 92%, 93%, 94%, 95%, 96%, 97%, 98%, 99%, or 100% identical to SEQ ID NO: 1275 (clone 17L). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1222; and a light chain variable region at least about 85%, 90%, 91%, 92%, 93%, 94%, 95%, 96%, 97%, 98%, 99%, or 100% identical to SEQ ID NO: 1275 (clone 17L-1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1223; and a light chain variable region at least about 85%, 90%, 91%, 92%, 93%, 94%, 95%, 96%, 97%, 98%, 99%, or 100% identical to SEQ ID NO: 202 (clone 23). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1224; and a light chain variable region at least about 85%, 90%, 91%, 92%, 93%, 94%, 95%, 96%, 97%, 98%, 99%, or 100% identical to SEQ ID NO: 202 (clone 53). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1224; and a light chain variable region at least about 85%, 90%, 91%, 92%, 93%, 94%, 95%, 96%, 97%, 98%, 99%, or 100% identical to SEQ ID NO: 1277 (clone E1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1225; and a light chain variable region at least about 85%, 90%, 91%, 92%, 93%, 94%, 95%, 96%, 97%, 98%, 99%, or 100% identical to SEQ ID NO: 1275 (clone 3-17L V-A). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1226; and a light chain variable region at least about 85%, 90%, 91%, 92%, 93%, 94%, 95%, 96%, 97%, 98%, 99%, or 100% identical to SEQ ID NO: 1275 (clone 3-17L). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1227; and a light chain variable region at least about 85%, 90%, 91%, 92%, 93%, 94%, 95%, 96%, 97%, 98%, 99%, or 100% identical to SEQ ID NO: 203 (clone L8mod). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1209; and a light chain variable region at least about 85%, 90%, 91%, 92%, 93%, 94%, 95%, 96%, 97%, 98%, 99%, or 100% identical to SEQ ID NO: 203 (clone L8). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1228; and a light chain variable region at least about 85%, 90%, 91%, 92%, 93%, 94%, 95%, 96%, 97%, 98%, 99%, or 100% identical to SEQ ID NO: 203 (clone X-V). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 203 (clone X). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 1278 (clone XL3-6). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 1279 (clone XL3-10). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 207 (clone XL3-15). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 204 (clone L3-13). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1230; and a light chain variable region at least about 85%, 90%, 91%, 92%, 93%, 94%, 95%, 96%, 97%, 98%, 99%, or 100% identical to SEQ ID NO: 203 (clone H3-1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1231; and a light chain variable region at least about 85%, 90%, 91%, 92%, 93%, 94%, 95%, 96%, 97%, 98%, 99%, or 100% identical to SEQ ID NO: 203 (clone H2-2). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1232; and a light chain variable region at least about 85%, 90%, 91%, 92%, 93%, 94%, 95%, 96%, 97%, 98%, 99%, or 100% identical to SEQ ID NO: 203 (clone H2-5). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1233; and a light chain variable region at least about 85%, 90%, 91%, 92%, 93%, 94%, 95%, 96%, 97%, 98%, 99%, or 100% identical to SEQ ID NO: 1280 (M1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1234; and a light chain variable region at least about 85%, 90%, 91%, 92%, 93%, 94%, 95%, 96%, 97%, 98%, 99%, or 100% identical to SEQ ID NO: 1281 (M2), In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1235; and a light chain variable region at least about 85%, 90%, 91%, 92%, 93%, 94%, 95%, 96%, 97%, 98%, 99%, or 100% identical to SEQ ID NO: 1282 (M3). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1235; and a light chain variable region at least about 85%, 90%, 91%, 92%, 93%, 94%, 95%, 96%, 97%, 98%, 99%, or 100% identical to SEQ ID NO: 1283 (M3). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1237; and a light chain variable region at least about 85%, 90%, 91%, 92%, 93%, 94%, 95%, 96%, 97%, 98%, 99%, or 100% identical to SEQ ID NO: 1284 (M4). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1238; and a light chain variable region at least about 85%, 90%, 91%, 92%, 93%, 94%, 95%, 96%, 97%, 98%, 99%, or 100% identical to SEQ ID NO: 1285 (M5). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1239-1242; and a light chain variable region at least about 85%, 90%, 91%, 92%, 93%, 94%, 95%, 96%, 97%, 98%, 99%, or 100% identical to any one of SEQ ID NOS: 1286-1293 (M6). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1243-1247; and a light chain variable region at least about 85%, 90%, 91%, 92%, 93%, 94%, 95%, 96%, 97%, 98%, 99%, or 100% identical to any one of SEQ ID NOS: 1294-1297 (M7). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1248-1259; and a light chain variable region at least about 85%, 90%, 91%, 92%, 93%, 94%, 95%, 96%, 97%, 98%, 99%, or 100% identical to any one of SEQ ID NOS: 1298-1312 (M8). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1260; and a light chain variable region at least about 85%, 90%, 91%, 92%, 93%, 94%, 95%, 96%, 97%, 98%, 99%, or 100% identical to SEQ ID NO: 1313 (M9). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1261; and a light chain variable region at least about 85%, 90%, 91%, 92%, 93%, 94%, 95%, 96%, 97%, 98%, 99%, or 100% identical to SEQ ID NO: 1314 (M10). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1262; and a light chain variable region at least about 85%, 90%, 91%, 92%, 93%, 94%, 95%, 96%, 97%, 98%, 99%, or 100% identical to SEQ ID NO: 1315 (M11). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1263; and a light chain variable region at least about 85%, 90%, 91%, 92%, 93%, 94%, 95%, 96%, 97%, 98%, 99%, or 100% identical to SEQ ID NO: 1316 (M12). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 301; and a light chain variable region at least about 85%, 90%, 91%, 92%, 93%, 94%, 95%, 96%, 97%, 98%, 99%, or 100% identical to SEQ ID NO: 303. In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 302; and a light chain variable region at least about 85%, 90%, 91%, 92%, 93%, 94%, 95%, 96%, 97%, 98%, 99%, or 100% identical to SEQ ID NO: 303. In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1301; and a light chain variable region at least about 85%, 90%, 91%, 92%, 93%, 94%, 95%, 96%, 97%, 98%, 99%, or 100% identical to SEQ ID NO: 303. In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1302; and a light chain variable region at least about 85%, 90%, 91%, 92%, 93%, 94%, 95%, 96%, 97%, 98%, 99%, or 100% identical to SEQ ID NO: 303.

### 5.3.1.4 Exemplary Anti-TL1A Constant Regions

In some embodiments, one or more amino acid modifications may be introduced into the Fragment crystallizable (Fc) region of a human or humanized antibody, thereby generating an Fc region variant. An Fc region may comprise a C-terminal region of an immunoglobulin heavy chain that comprises a binge region, CH2 domain, CH3 domain, or any combination thereof. As used herein, an Fc region includes native sequence Fc regions and variant Fe regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions. In an exemplary embodiment, the Fc region comprises any one of SEQ ID NOS: 320-362, 401-413, 501-515.

In some embodiments, antibodies of this disclosure have a reduced effector function as compared to a human IgG. Effector function refers to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation. In some cases, antibody-dependent cell-mediated cytotoxicity (ADCC) refers to a cell-mediated reaction in which nonspecific cytotoxic cells expressing Fe receptors (e.g., natural killer cells, neutrophils, macrophages) recognize bound antibody on a target cell, subsequently causing lysis of the target cell. In some cases, complement dependent cytotoxicity (CDC) refers to lysing of a target cell in the presence of complement, where the complement action pathway is initiated by the binding of C1q to antibody bound with the target.

Some Fc regions have a natural lack of effector function, and some Fc regions can comprise mutations that reduce effector functions. For instance, IgG4 has low ADCC and CDC activities and IgG2 has low ADCC activity.

The disclosure provides antibodies comprising Fc regions characterized by exhibiting ADCC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease ADCC (such as human IgG1, SEQ ID NO: 320). The disclosure provides antibodies comprising Fc regions characterized by exhibiting CDC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease CDC (such as human IgG1, SEQ ID NO: 320). In certain embodiments, the antibodies of this disclosure have reduced effector function as compared with human IgG1. Measurement of effector function may be performed as described in Example 3.

Non-limiting examples of Fc mutations in IgG1 that may reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat. In certain embodiments, the antibodies of this disclosure have reduced effector function as compared with human IgG1.

In some embodiments, an antibody comprises an IgG1 Fc region comprising an N297A substitution, according to the Kabat numbering system. In some embodiments, an antibody comprises an IgG1 Fc region comprising an N297Q substitution, according to the Kabat numbering system. In some embodiments, an antibody comprises an IgG1 Fe region comprising an N297D substitution, according to the Kabat numbering system. In some embodiments, an antibody comprises an IgG1 Fc region comprising an D265A substitution, according to the Kabat numbering system. In some embodiments, an antibody comprises an IgG1 Fe region comprising an S228P substitution, according to the Kabat numbering system. In some embodiments, an antibody comprises an IgG1 Fc region comprising an L235A substitution, according to the Kabat numbering system. In some embodiments, an antibody comprises an IgG1 Fe region comprising an L237A substitution, according to the Kabat numbering system. In some embodiments, an antibody comprises an IgG1 Fc region comprising an L234A substitution, according to the Kabat numbering system. In some embodiments, an antibody comprises an IgG1 Fc region comprising an E233P substitution, according to the Kabat numbering system. In some embodiments, an antibody comprises an IgG1 Fe region comprising an L234V substitution, according to the Kabat numbering system. In some embodiments, an antibody comprises an IgG1 Fc region comprising an C236 deletion, according to the Kabat numbering system. In some embodiments, an antibody comprises an IgG1 Fc region comprising a P238A substitution, according to the Kabat numbering system. In some embodiments, an antibody comprises an IgG1 Fc region comprising an A327Q substitution, according to the Kabat numbering system. In some embodiments, an antibody comprises an IgG1 Fc region comprising a P329A substitution, according to the Kabat numbering system. In some embodiments, an antibody comprises an IgG1 Fe region comprising an P329G substitution, according to the Kabat numbering system. In some embodiments, an antibody comprises an IgG1 Fc region comprising an L23SE substitution, according to the Kabat numbering system. In some embodiments, an antibody comprises an IgG1 Fc region comprising an P331S substitution, according to the Kabat numbering system. In some embodiments, an antibody comprises an IgG1 Fc region comprising an L234F substitution, according to the Kabat numbering system. In some embodiments, an antibody comprises an IgG1 Fe region comprising a 235G substitution, according to the Kabat numbering system. In some embodiments, an antibody comprises an IgG1 Fe region comprising an 235Q substitution, according to the Kabat numbering system. In some embodiments, an antibody comprises an IgG1 Fc region comprising an 235R substitution, according to the Kabat numbering system. In some embodiments, an antibody comprises an IgG1 Fc region comprising an 235S substitution, according to the Kabat numbering system. In some embodiments, an antibody comprises an IgG1 Fc region comprising an 236F substitution, according to the Kabat numbering system. In some embodiments, an antibody comprises an IgG1 Fc region comprising an 236R substitution, according to the Kabat numbering system. In some embodiments, an antibody comprises an IgG1 Fe region comprising an 237E substitution, according to the Kabat numbering system. In some embodiments, an antibody comprises an IgG1 Fc region comprising an 237K substitution, according to the Kabat numbering system. In some embodiments, an antibody comprises an IgG1 Fc region comprising an 237N substitution, according to the Kabat numbering system. In some embodiments, an antibody comprises an IgG1 Fe region comprising an 237R substitution, according to the Kabat numbering system. In some embodiments, an antibody comprises an IgG1 Fc region comprising an 238A substitution, according to the Kabat numbering system. In some embodiments, an antibody comprises an IgG1 Fc region comprising an 238E substitution, according to the Kabat numbering system. In some embodiments, an antibody comprises an IgG1 Fc region comprising an 238G substitution, according to the Kabat numbering system. In some embodiments, an antibody comprises an IgG1 Fc region comprising an 238H substitution, according to the Kabat numbering system. In some embodiments, an antibody comprises an IgG1 Fc region comprising an 2381 substitution, according to the Kabat numbering system. In some embodiments, an antibody comprises an IgG1 Fc region comprising an 238V substitution, according to the Kabat numbering system. In some embodiments, an antibody comprises an IgG1 Fc region comprising an 238 W substitution, according to the Kabat numbering system. In some embodiments, an antibody comprises an IgG1 Fc region comprising an 238Y substitution, according to the Kabat numbering system. In some embodiments, an antibody comprises an IgG1 Fc region comprising an 248A substitution, according to the Kabat numbering system. In some embodiments, an antibody comprises an IgG1 Fc region comprising an 254D substitution, according to the Kabat numbering system. In some embodiments, an antibody comprises an IgG1 Fc region comprising an 254E substitution, according to the Kabat numbering system. In some embodiments, an antibody comprises an IgG1 Fc region comprising an 254G substitution, according to the Kabat numbering system. In some embodiments, an antibody comprises an IgG1 Fe region comprising an 254H substitution, according to the Kabat numbering system. In some embodiments, an antibody comprises an IgG1 Fc region comprising an 2541 substitution, according to the Kabat numbering system. In some embodiments, an antibody comprises an IgG1 Fc region comprising an 254N substitution, according to the Kabat numbering system. In some embodiments, an antibody comprises an IgG1 Fc region comprising an 254P substitution, according to the Kabat numbering system. In some embodiments, an antibody comprises an IgG1 Fc region comprising an 254Q substitution, according to the Kabat numbering system. In some embodiments, an antibody comprises an IgG1 Fc region comprising an 254T substitution, according to the Kabat numbering system. In some embodiments, an antibody comprises an IgG1 Fc region comprising an 254V substitution, according to the Kabat numbering system. In some embodiments, an antibody comprises an IgG1 Fc region comprising an 255N substitution, according to the Kabat numbering system. In some embodiments, an antibody comprises an IgG1 Fc region comprising an 256H substitution, according to the Kabat numbering system. In some embodiments, an antibody comprises an IgG1 Fc region comprising an 256K substitution, according to the Kabat numbering system. In some embodiments, an antibody comprises an IgG1 Fc region comprising an 256R substitution, according to the Kabat numbering system. In some embodiments, an antibody comprises an IgG1 Fc region comprising an 256V substitution, according to the Kabat numbering system. In some embodiments, an antibody comprises an IgG1 Fe region comprising an 264S substitution, according to the Kabat numbering system. In some embodiments, an antibody comprises an IgG1 Fc region comprising an 265H substitution, according to the Kabat numbering system. In some embodiments, an antibody comprises an IgG1 Fc region comprising an 265K substitution, according to the Kabat numbering system. In some embodiments, an antibody comprises an IgG1 Fc region comprising an 265S substitution, according to the Kabat numbering system. In some embodiments, an antibody comprises an IgG1 Fe region comprising an 265Y substitution, according to the Kabat numbering system. In some embodiments, an antibody comprises an IgG1 Fc region comprising an 267G substitution, according to the Kabat numbering system. In some embodiments, an antibody comprises an IgG1 Fc region comprising an 267H substitution, according to the Kabat numbering system. In some embodiments, an antibody comprises an IgG1 Fc region comprising an 2671 substitution, according to the Kabat numbering system. In some embodiments, an antibody comprises an IgG1 Fc region comprising an 267K substitution, according to the Kabat numbering system. In some embodiments, an antibody comprises an IgG1 Fc region comprising an 268K substitution, according to the Kabat numbering system. In some embodiments, an antibody comprises an IgG1 Fc region comprising an 269N substitution, according to the Kabat numbering system. In some embodiments, an antibody comprises an IgG1 Fc region comprising an 269Q substitution, according to the Kabat numbering system. In some embodiments, an antibody comprises an IgG1 Fc region comprising an 270A substitution, according to the Kabat numbering system. In some embodiments, an antibody comprises an IgG1 Fc region comprising an 270G substitution, according to the Kabat numbering system. In some embodiments, an antibody comprises an IgG1 Fe region comprising an 270M substitution, according to the Kabat numbering system. In some embodiments, an antibody comprises an IgG1 Fc region comprising an 270N substitution, according to the Kabat numbering system. In some embodiments, an antibody comprises an IgG1 Fc region comprising an 271T substitution, according to the Kabat numbering system. In some embodiments, an antibody comprises an IgG1 Fc region comprising an 272N substitution, according to the Kabat numbering system. In some embodiments, an antibody comprises an IgG1 Fe region comprising an 279F substitution, according to the Kabat numbering system. In some embodiments, an antibody comprises an IgG1 Fc region comprising an 279K substitution, according to the Kabat numbering system. In some embodiments, an antibody comprises an IgG1 Fc region comprising an 279L substitution, according to the Kabat numbering system. In some embodiments, an antibody comprises an IgG1 Fe region comprising an 292E substitution, according to the Kabat numbering system. In some embodiments, an antibody comprises an IgG1 Fc region comprising an 292F substitution, according to the Kabat numbering system. In some embodiments, an antibody comprises an IgG1 Fc region comprising an 292G substitution, according to the Kabat numbering system. In some embodiments, an antibody comprises an IgG1 Fc region comprising an 2921 substitution, according to the Kabat numbering system. In some embodiments, an antibody comprises an IgG1 Fc region comprising an 293S substitution, according to the Kabat numbering system. In some embodiments, an antibody comprises an IgG1 Fe region comprising an 301 W substitution, according to the Kabat numbering system. In some embodiments, an antibody comprises an IgG1 Fc region comprising an 304E substitution, according to the Kabat numbering system. In some embodiments, an antibody comprises an IgG1 Fc region comprising an 311E substitution, according to the Kabat numbering system. In some embodiments, an antibody comprises an IgG1 Fc region comprising an 311G substitution, according to the Kabat numbering system. In some embodiments, an antibody comprises an IgG1 Fe region comprising an 311S substitution, according to the Kabat numbering system. In some embodiments, an antibody comprises an IgG1 Fc region comprising an 316F substitution, according to the Kabat numbering system. In some embodiments, an antibody comprises an IgG1 Fc region comprising an 327T substitution, according to the Kabat numbering system. In some embodiments, an antibody comprises an IgG1 Fc region comprising an 328V substitution, according to the Kabat numbering system. In some embodiments, an antibody comprises an IgG1 Fc region comprising an 329Y substitution, according to the Kabat numbering system. In some embodiments, an antibody comprises an IgG1 Fc region comprising an 330R substitution, according to the Kabat numbering system. In some embodiments, an antibody comprises an IgG1 Fc region comprising an 339E substitution, according to the Kabat numbering system. In some embodiments, an antibody comprises an IgG1 Fc region comprising an 339L substitution, according to the Kabat numbering system. In some embodiments, an antibody comprises an IgG1 Fc region comprising an 3431 substitution, according to the Kabat numbering system. In some embodiments, an antibody comprises an IgG1 Fc region comprising an 343V substitution, according to the Kabat numbering system. In some embodiments, an antibody comprises an IgG1 Fc region comprising an 373A substitution, according to the Kabat numbering system. In some embodiments, an antibody comprises an IgG1 Fc region comprising an 373G substitution, according to the Kabat numbering system. In some embodiments, an antibody comprises an IgG1 Fc region comprising an 373S substitution, according to the Kabat numbering system. In some embodiments, an antibody comprises an IgG1 Fc region comprising an 376E substitution, according to the Kabat numbering system. In some embodiments, an antibody comprises an IgG1 Fc region comprising an 376 W substitution, according to the Kabat numbering system. In some embodiments, an antibody comprises an IgG1 Fc region comprising an 376Y substitution, according to the Kabat numbering system. In some embodiments, an antibody comprises an IgG1 Fc region comprising an 380D substitution, according to the Kabat numbering system. In some embodiments, an antibody comprises an IgG1 Fc region comprising an 382D substitution, according to the Kabat numbering system. In some embodiments, an antibody comprises an IgG1 Fc region comprising an 382P substitution, according to the Kabat numbering system. In some embodiments, an antibody comprises an IgG1 Fc region comprising an 385P substitution, according to the Kabat numbering system. In some embodiments, an antibody comprises an IgG1 Fc region comprising an 424H substitution, according to the Kabat numbering system. In some embodiments, an antibody comprises an IgG1 Fc region comprising an 424M substitution, according to the Kabat numbering system. In some embodiments, an antibody comprises an IgG1 Fc region comprising an 424V substitution, according to the Kabat numbering system. In some embodiments, an antibody comprises an IgG1 Fc region comprising an 4341 substitution, according to the Kabat numbering system. In some embodiments, an antibody comprises an IgG1 Fc region comprising an 438G substitution, according to the Kabat numbering system. In some embodiments, an antibody comprises an IgG1 Fc region comprising an 439E substitution, according to the Kabat numbering system. In some embodiments, an antibody comprises an IgG1 Fe region comprising an 439H substitution, according to the Kabat numbering system. In some embodiments, an antibody comprises an IgG1 Fc region comprising an 439Q substitution, according to the Kabat numbering system. In some embodiments, an antibody comprises an IgG1 Fc region comprising an 440A substitution, according to the Kabat numbering system. In some embodiments, an antibody comprises an IgG1 Fc region comprising an 440D substitution, according to the Kabat numbering system. In some embodiments, an antibody comprises an IgG1 Fc region comprising an 440E substitution, according to the Kabat numbering system. In some embodiments, an antibody comprises an IgG1 Fc region comprising an 440F substitution, according to the Kabat numbering system. In some embodiments, an antibody comprises an IgG1 Fc region comprising an 440M substitution, according to the Kabat numbering system. In some embodiments, an antibody comprises an IgG1 Fc region comprising an 440T Fc region substitution, according to the Kabat numbering system. In some embodiments, an antibody comprises an IgG1 Fc region comprising an 440V substitution, according to the Kabat numbering system.

In some embodiments, an antibody comprises a Fe region selected from the representative sequences disclosed in Table 3 or 20 In some embodiments, an antibody comprises an IgG1 Fc region comprising E233P, according to the Kabat numbering system. In some embodiments, an antibody comprises an IgG4 Fc region comprising S228P and L235E. In some embodiments, an antibody comprises an IgG1 Fc region comprising L235E, according to the Kabat numbering system. In some embodiments, an antibody comprises an IgG1 Fc region comprising L234A and L235A, according to the Kabat numbering system. In some embodiments, an antibody comprises an IgG1 Fe region comprising L234A, L23SA, and G237A, according to the Kabat numbering system. In some embodiments, an antibody comprises an IgG1 Fc region comprising L234A, L235A, P329G, according to the Kabat numbering system. In some embodiments, an antibody comprises an IgG1 Fc region comprising L234F, L235E, and P331S, according to the Kabat numbering system. In some embodiments, an antibody comprises an IgG1 Fc region comprising L234A, L235E, and G237A, according to the Kabat numbering system. In some embodiments, an antibody comprises an IgG1 Fc region comprising L234A, L235E, G237A, and P331S, according to the Kabat numbering system. In some embodiments, an antibody comprises an IgG1 Fc region comprising L234A, L235A, G237A, P238S, H268A, A330S, and P331S (IgGlo), according to the Kabat numbering system. In some embodiments, an antibody comprises an IgG1 Fc region comprising L234A, L23SA, and P329A, according to the Kabat numbering system. In some embodiments, an antibody comprises an IgG1 Fc region comprising G236R and L328R, according to the Kabat numbering system. In some embodiments, an antibody comprises an IgG1 Fc region comprising G237A, according to the Kabat numbering system. In some embodiments, an antibody comprises an IgG1 Fc region comprising F241A, according to the Kabat numbering system. In some embodiments, an antibody comprises an IgG1 Fc region comprising V264A, according to the Kabat numbering system. In some embodiments, an antibody comprises an IgG1 Fc region comprising D265A, according to the Kabat numbering system. In some embodiments, an antibody comprises an IgG1 Fc region comprising D265A and N297A, according to the Kabat numbering system. In some embodiments, an antibody comprises an IgG1 Fc region comprising D265A and N297G, according to the Kabat numbering system. In some embodiments, an antibody comprises an IgG1 Fc region comprising D270A, according to the Kabat numbering system. In some embodiments, an antibody comprises an IgG1 Fc region comprising N297A, according to the Kabat numbering system. In some embodiments, an antibody comprises an IgG1 Fc region comprising N297G, according to the Kabat numbering system. In some embodiments, an antibody comprises an IgG1 Fc region comprising N297D, according to the Kabat numbering system. In some embodiments, an antibody comprises an IgG1 Fc region comprising N297Q, according to the Kabat numbering system. In some embodiments, an antibody comprises an IgG1 Fc region comprising P329A, according to the Kabat numbering system. In some embodiments, an antibody comprises an IgG1 Fc region comprising P329G, according to the Kabat numbering system. In some embodiments, an antibody comprises an IgG1 Fc region comprising P329R, according to the Kabat numbering system. In some embodiments, an antibody comprises an IgG1 Fc region comprising A330L, according to the Kabat numbering system. In some embodiments, an antibody comprises an IgG1 Fc region comprising P331A, according to the Kabat numbering system. In some embodiments, an antibody comprises an IgG1 Fc region comprising P331S, according to the Kabat numbering system. In some embodiments, an antibody comprises an IgG2 Fe region. In some embodiments, an antibody comprises an IgG4 Fc region. In some embodiments, an antibody comprises an IgG4 Fc region comprising S228P, according to the Kabat numbering system. In some embodiments, an antibody comprises an IgG4 Fc region comprising S228P, F234A, and L235A, according to the Kabat numbering system. In some embodiments, an antibody comprises an IgG2-IgG4 cross-subclass (IgG2/G4) Fc region. In some embodiments, an antibody comprises an IgG2-IgG3 cross-subclass Fc region. In some embodiments, an antibody comprises an IgG2 Fc region comprising H268Q, V309L, A330S, and P331S, according to the Kabat numbering system. In some embodiments, an antibody comprises an IgG2 Fc region comprising V234A, G237A, P238S. H268A, V309L, A330S, and P331S, according to the Kabat numbering system. In some embodiments, an antibody comprises a Fc region comprising high mannose glycosylation.

In some embodiments, an antibody comprises an IgG4 Fc region comprising a S228P substitution, according to the Kabat numbering system. In some embodiments, an antibody comprises an IgG4 Fc region comprising an A330S substitution, according to the Kabat numbering system. In some embodiments, an antibody comprises an IgG4 Fc region comprising a P331S substitution, according to the Kabat numbering system.

In some embodiments, an antibody comprises an IgG2 Fc region comprising an A330S substitution, according to the Kabat numbering system. In some embodiments, an antibody comprises an IgG2 Fc region comprising an P331S substitution, according to the Kabat numbering system. In some embodiments, an antibody comprises an IgG2 Fc region comprising an 234A substitution, according to the Kabat numbering system. In some embodiments, an antibody comprises an IgG2 Fc region comprising an 237A substitution, according to the Kabat numbering system.

In certain embodiments, an anti-TL1A antibody described herein comprises a Fc region comprising a sequence from Table 19. In certain embodiments, an anti-TL1A described herein comprises a Fc region as shown in Table 3.

TABLE 3
Exemplary Fc Mutations

| | Constant Region (SEQ ID NO) | | |
|---|---|---|---|
| Mutations | K_DL | R_EM | K_EM |
| Wild-type IgG1 | 320 | 321 | 322 |
| L235E | 323 | 324 | 325 |
| L234A, L235A | 326 | 327 | 328 |
| L234A, L235A, G237A | 329 | 330 | 331 |
| L234A, L235A, P329G | 332 | 333 | 334 |
| L234F, L235E, P331S | 335 | 336 | 337 |
| L234A, L235E, G237A | 338 | 339 | 340 |
| L234A, L235E, G237A, P331S | 341 | 342 | 343 |
| L234A, L235A, P329A | 344 | 345 | 346 |
| D265A | 347 | 348 | 349 |
| N297G | 350 | 351 | 352 |
| D265A, N297A | 353 | 354 | 355 |
| D265A, N297G | 356 | 357 | 358 |
| L235A, G237A | 359 | 360 | 361 |
| Wild-type IgG4 | 362 | | |

In certain embodiments, an anti-TL1A antibody described herein comprises a Fc region comprising SEQ ID NO: 320 or a sequence at least about 90%, 91%, 92%, 93%, 94%, 95%, 96%, 97%, 98%, or 99% identical to SEQ ID NO: 320. In certain embodiments, an anti-TL1A antibody described herein comprises a Fc region comprising SEQ ID NO: 321 or a sequence at least about 90%, 91%, 92%, 93%, 94%, 95%, 96%, 97%, 98%, or 99% identical to SEQ ID NO: 321. In certain embodiments, an anti-TL1A antibody described herein comprises a Fc region comprising SEQ ID NO: 322 or a sequence at least about 90%, 91%, 92%, 93%, 94%, 95%, 96%, 97%, 98%, or 99% identical to SEQ ID NO: 322. In certain embodiments, an anti-TL1A antibody described herein comprises a Fc region comprising SEQ ID NO: 323 or a sequence at least about 90%, 91%, 92%, 93%, 94%, 95%, 96%, 97%, 98%, or 99% identical to SEQ ID NO: 323. In certain embodiments, an anti-TL1A antibody described herein comprises a Fc region comprising SEQ ID NO: 324 or a sequence at least about 90%, 91%, 92%, 93%, 94%, 95%, 96%, 97%, 98%, or 99% identical to SEQ ID NO: 324. In certain embodiments, an anti-TL1A antibody described herein comprises a Fc region comprising SEQ ID NO: 325 or a sequence at least about 90%, 91%, 92%, 93%, 94%, 95%, 96%, 97%, 98%, or 99% identical to SEQ ID NO: 325. In certain embodiments, an anti-TL1A antibody described herein comprises a Fc region comprising SEQ ID NO: 326 or a sequence at least about 90%, 91%, 92%, 93%, 94%, 95%, 96%, 97%, 98%, or 99% identical to SEQ ID NO: 326. In certain embodiments, an anti-TL1A antibody described herein comprises a Fc region comprising SEQ ID NO: 327 or a sequence at least about 90%, 91%, 92%, 93%, 94%, 95%, 96%, 97%, 98%, or 99% identical to SEQ ID NO: 327. In certain embodiments, an anti-TL1A antibody described herein comprises a Fe region comprising SEQ ID NO: 328 or a sequence at least about 90%, 91%, 92%, 93%, 94%, 95%, 96%, 97%, 98%, or 99% identical to SEQ ID NO: 328. In certain embodiments, an anti-TL1A antibody described herein comprises a Fc region comprising SEQ ID NO: 329 or a sequence at least about 90%, 91%, 92%, 93%, 94%, 95%, 96%, 97%, 98%, or 99% identical to SEQ ID NO: 329. In certain embodiments, an anti-TL1A antibody described herein comprises a Fc region comprising SEQ ID NO: 330 or a sequence at least about 90%, 91%, 92%, 93%, 94%, 95%, 96%, 97%, 98%, or 99% identical to SEQ ID NO: 330. In certain embodiments, an anti-TL1A antibody described herein comprises a Fc region comprising SEQ ID NO: 331 or a sequence at least about 90%, 91%, 92%, 93%, 94%, 95%, 96%, 97%, 98%, or 99% identical to SEQ ID NO: 331. In certain embodiments, an anti-TL1A antibody described herein comprises a Fc region comprising SEQ ID NO: 332 or a sequence at least about 90%, 91%, 92%, 93%, 94%, 95%, 96%, 97%, 98%, or 99% identical to SEQ ID NO: 332. In certain embodiments, an anti-TL1A antibody described herein comprises a Fc region comprising SEQ ID NO: 333 or a sequence at least about 90%, 91%, 92%, 93%, 94%, 95%, 96%, 97%, 98%, or 99% identical to SEQ ID NO: 333. In certain embodiments, an anti-TL1A antibody described herein comprises a Fc region comprising SEQ ID NO: 334 or a sequence at least about 90%, 91%, 92%, 93%, 94%, 95%, 96%, 97%, 98%, or 99% identical to SEQ ID NO: 334. In certain embodiments, an anti-TL1A antibody described herein comprises a Fc region comprising SEQ ID NO: 335 or a sequence at least about 90%, 91%, 92%, 93%, 94%, 95%, 96%, 97%, 98%, or 99% identical to SEQ ID NO: 335. In certain embodiments, an anti-TL1A antibody described herein comprises a Fc region comprising SEQ ID NO: 336 or a sequence at least about 90%, 91%, 92%, 93%, 94%, 95%, 96%, 97%, 98%, or 99% identical to SEQ ID NO: 336. In certain embodiments, an anti-TL1A antibody described herein comprises a Fc region comprising SEQ ID NO: 337 or a sequence at least about 90%, 91%, 92%, 93%, 94%, 95%, 96%, 97%, 98%, or 99% identical to SEQ ID NO: 337. In certain embodiments, an anti-TL1A antibody described herein comprises a Fc region comprising SEQ ID NO: 338 or a sequence at least about 90%, 91%, 92%, 93%, 94%, 95%, 96%, 97%, 98%, or 99% identical to SEQ ID NO: 338. In certain embodiments, an anti-TL1A antibody described herein comprises a Fc region comprising SEQ ID NO: 339 or a sequence at least about 90%, 91%, 92%, 93%, 94%, 95%, 96%, 97%, 98%, or 99% identical to SEQ ID NO: 339. In certain embodiments, an anti-TL1A antibody described herein comprises a Fc region comprising SEQ ID NO: 340 or a sequence at least about 90%, 91%, 92%, 93%, 94%, 95%, 96%, 97%, 98%, or 99% identical to SEQ ID NO: 340. In certain embodiments, an anti-TL1A antibody described herein comprises a Fc region comprising SEQ ID NO: 341 or a sequence at least about 90%, 91%, 92%, 93%, 94%, 95%, 96%, 97%, 98%, or 99% identical to SEQ ID NO: 341. In certain embodiments, an anti-TL1A antibody described herein comprises a Fc region comprising SEQ ID NO: 342 or a sequence at least about 90%, 91%, 92%, 93%, 94%, 95%, 96%, 97%, 98%, or 99% identical to SEQ ID NO: 342. In certain embodiments, an anti-TL1A antibody described herein comprises a Fe region comprising SEQ ID NO: 343 or a sequence at least about 90%, 91%, 92%, 93%, 94%, 95%, 96%, 97%, 98%, or 99% identical to SEQ ID NO: 343. In certain embodiments, an anti-TL1A antibody described herein comprises a Fc region comprising SEQ ID NO: 344 or a sequence at least about 90%, 91%, 92%, 93%, 94%, 95%, 96%, 97%, 98%, or 99% identical to SEQ ID NO: 344. In certain embodiments, an anti-TL1A antibody described herein comprises a Fc region comprising SEQ ID NO: 345 or a sequence at least about 90%, 91%, 92%, 93%, 94%, 95%, 96%, 97%, 98%, or 99% identical to SEQ ID NO: 345. In certain embodiments, an anti-TL1A antibody described herein comprises a Fc region comprising SEQ ID NO: 346 or a sequence at least about 90%, 91%, 92%, 93%, 94%, 95%, 96%, 97%, 98%, or 99% identical to SEQ ID NO: 346. In certain embodiments, an anti-TL1A antibody described herein comprises a Fc region comprising SEQ ID NO: 347 or a sequence at least about 90%, 91%, 92%, 93%, 94%, 95%, 96%, 97%, 98%, or 99% identical to SEQ ID NO: 347. In certain embodiments, an anti-TL1A antibody described herein comprises a Fc region comprising SEQ ID NO: 348 or a sequence at least about 90%, 91%, 92%, 93%, 94%, 95%, 96%, 97%, 98%, or 99% identical to SEQ ID NO: 348. In certain embodiments, an anti-TL1A antibody described herein comprises a Fc region comprising SEQ ID NO: 349 or a sequence at least about 90%, 91%, 92%, 93%, 94%, 95%, 96%, 97%, 98%, or 99% identical to SEQ ID NO: 349. In certain embodiments, an anti-TL1A antibody described herein comprises a Fc region comprising SEQ ID NO: 350 or a sequence at least about 90%, 91%, 92%, 93%, 94%, 95%, 96%, 97%, 98%, or 99% identical to SEQ ID NO: 350. In certain embodiments, an anti-TL1A antibody described herein comprises a Fc region comprising SEQ ID NO: 351 or a sequence at least about 90%, 91%, 92%, 93%, 94%, 95%, 96%, 97%, 98%, or 99% identical to SEQ ID NO: 351. In certain embodiments, an anti-TL1A antibody described herein comprises a Fc region comprising SEQ ID NO: 352 or a sequence at least about 90%, 91%, 92%, 93%, 94%, 95%, 96%, 97%, 98%, or 99% identical to SEQ ID NO: 352. In certain embodiments, an anti-TL1A antibody described herein comprises a Fc region comprising SEQ ID NO: 353 or a sequence at least about 90%, 91%, 92%, 93%, 94%, 95%, 96%, 97%, 98%, or 99% identical to SEQ ID NO: 353. In certain embodiments, an anti-TL1A antibody described herein comprises a Fc region comprising SEQ ID NO: 354 or a sequence at least about 90%, 91%, 92%, 93%, 94%, 95%, 96%, 97%, 98%, or 99% identical to SEQ ID NO: 354. In certain embodiments, an anti-TL1A antibody described herein comprises a Fc region comprising SEQ ID NO: 355 or a sequence at least about 90%, 91%, 92%, 93%, 94%, 95%, 96%, 97%, 98%, or 99% identical to SEQ ID NO: 355. In certain embodiments, an anti-TL1A antibody described herein comprises a Fc region comprising SEQ ID NO: 356 or a sequence at least about 90%, 91%, 92%, 93%, 94%, 95%, 96%, 97%, 98%, or 99% identical to SEQ ID NO: 356. In certain embodiments, an anti-TL1A antibody described herein comprises a Fc region comprising SEQ ID NO: 357 or a sequence at least about 90%, 91%, 92%, 93%, 94%, 95%, 96%, 97%, 98%, or 99% identical to SEQ ID NO: 357. In certain embodiments, an anti-TL1A antibody described herein comprises a Fc region comprising SEQ ID NO: 358 or a sequence at least about 90%, 91%, 92%, 93%, 94%, 95%, 96%, 97%, 98%, or 99% identical to SEQ ID NO: 358. In certain embodiments, an anti-TL1A antibody described herein comprises a Fc region comprising SEQ ID NO: 359 or a sequence at least about 90%, 91%, 92%, 93%, 94%, 95%, 96%, 97%, 98%, or 99% identical to SEQ ID NO: 359. In certain embodiments, an anti-TL1A antibody described herein comprises a Fc region comprising SEQ ID NO: 360 or a sequence at least about 90%, 91%, 92%, 93%, 94%, 95%, 96%, 97%, 98%, or 99% identical to SEQ ID NO: 360. In certain embodiments, an anti-TL1A antibody described herein comprises a Fc region comprising SEQ ID NO: 361 or a sequence at least about 90%, 91%, 92%, 93%, 94%, 95%, 96%, 97%, 98%, or 99% identical to SEQ ID NO: 361. In certain embodiments, an anti-TL1A antibody described herein comprises a Fc region comprising SEQ ID NO: 362 or a sequence at least about 90%, 91%, 92%, 93%, 94%, 95%, 96%, 97%, 98%, or 99% identical to SEQ ID NO: 362.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious.

In vitro and/or in vivo cytotoxicity assays can be conducted to confirm the reduction/depletion of CDC and/or ADCC activities. For example, Fe receptor (FcR) binding assays can be conducted to ensure that the antibody lacks FcγR binding (hence likely lacking ADCC activity) but retains FcRn binding ability. Measurement of effector function may be performed as described in Example 3.

In some embodiments, antibodies are tested for binding to Fcγ receptors and complement C1q by ELISA. In some embodiments, antibodies are tested for the ability to activate primary human immune cells in vitro, for example, by assessing their ability to induce expression of activation markers.

In some embodiments, assessment of ADCC activity of an anti-TL1A antibody comprises adding the antibody to target cells in combination with immune effector cells, which may be activated by the antigen antibody complexes resulting in cytolysis of the target cell. Cytolysis may be detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38. Alternatively, or additionally, ADCC activity of the antibody of interest may be assessed in vivo, e.g., in an animal model such as that disclosed in Clynes et al., 1998, PNAS USA 95:652-656.

In some embodiments, antibodies comprising a Fc region herein exhibit decreased ADCC activities as compared to an unmodified antibody (e.g., an antibody with human IgG1). In some embodiments, the antibodies herein exhibit ADCC activities that are at least 2-fold, or at least 3-fold, or at least 5-fold or at least 10-fold or at least 50-fold or at least 100-fold less than that of an unmodified antibody. In some embodiments, antibodies herein exhibit ADCC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein have no detectable ADCC activity. In certain embodiments, the reduction and/or abatement of ADCC activity may be attributed to the reduced affinity antibodies of the invention exhibit for Fc ligands and/or receptors.

In some embodiments, an assessment of complement activation, a CDC assay, may be performed as described in Gazzano-Santoro et al., 1996, J. Immunol, Methods, 202:163.

In some embodiments, antibodies comprising Fc regions described herein exhibit decreased affinities to C1q relative to an unmodified antibody (e.g., human IgG1). In some embodiments, antibodies herein exhibit affinities for C1q receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than an unmodified antibody. In some embodiments, antibodies herein exhibit affinities for C1q that are at least 90%, at least 80%, at least 70%, at least 60%, at least 50%, at least 40%, at least 30%, at least 20%, at least 10%, or at least 5% less than an unmodified antibody. In some embodiments, antibodies herein exhibit affinities for C1q that are between about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 AM to about 100 μM. In certain embodiments, antibodies herein exhibit affinities for C1q that are greater than 1 μM, greater than 5 μM, greater than 10 μM, greater than 25 μM, greater than 50 μM, or greater than 100 μM.

In some embodiments, antibodies comprising Fc regions described herein exhibit decreased CDC activities as compared to an unmodified antibody (e.g., human IgG1). In some embodiments, antibodies herein exhibit CDC activities that are at least 2-fold, or at least 3-fold, or at least 5-fold or at least 10-fold or at least 50-fold or at least 100-fold less than that of an unmodified antibody. In some embodiments, antibodies herein exhibit CDC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein exhibit no detectable CDC activities. In some embodiments, the reduction and/or abatement of CDC activity may be attributed to the reduced affinity antibodies of the invention exhibit for Fe ligands and/or receptors.

Accordingly, further provided and described herein are anti-TL1A antibodies comprising a variant (e.g. harboring mutations) Fc region that reduce the cytotoxic response (e.g. ADCC or CDC) elicited by an anti-TL1A antibody. In some embodiments, an anti-TL1A antibody described herein comprises a Fc region comprising SEQ ID NO: 401 or a sequence at least about 90%, 91%, 92%, 93%, 94%, 95%, 96%, 97%, 98%, or 99% identical to SEQ ID NO: 401. In some embodiments, an anti-TL1A antibody described herein comprises a Fc region comprising SEQ ID NO: 402 or a sequence at least about 90%, 91%, 92%, 93%, 94%, 95%, 96%, 97%, 98%, or 99% identical to SEQ ID NO: 402. In some embodiments, an anti-TL1A antibody described herein comprises a Fc region comprising SEQ ID NO: 403 or a sequence at least about 90%, 91%, 92%, 93%, 94%, 95%, 96%, 97%, 98%, or 99% identical to SEQ ID NO: 403. In some embodiments, an anti-TL1A antibody described herein comprises a Fc region comprising SEQ ID NO: 404 or a sequence at least about 90%, 91%, 92%, 93%, 94%, 95%, 96%, 97%, 98%, or 99% identical to SEQ ID NO: 404. In some embodiments, an anti-TL1A antibody described herein comprises a Fc region comprising SEQ ID NO: 405 or a sequence at least about 90%, 91%, 92%, 93%, 94%, 95%, 96%, 97%, 98%, or 99% identical to SEQ ID NO: 405. In some embodiments, an anti-TL1A antibody described herein comprises a Fc region comprising SEQ ID NO: 406 or a sequence at least about 90%, 91%, 92%, 93%, 94%, 95%, 96%, 97%, 98%, or 99% identical to SEQ ID NO: 406. In some embodiments, an anti-TL1A antibody described herein comprises a Fc region comprising SEQ ID NO: 407 or a sequence at least about 90%, 91%, 92%, 93%, 94%, 95%, 96%, 97%, 98%, or 99% identical to SEQ ID NO: 407. In some embodiments, an anti-TL1A antibody described herein comprises a Fc region comprising SEQ ID NO: 408 or a sequence at least about 90%, 91%, 92%, 93%, 94%, 95%, 96%, 97%, 98%, or 99% identical to SEQ ID NO: 408. In some embodiments, an anti-TL1A antibody described herein comprises a Fc region comprising SEQ ID NO: 409 or a sequence at least about 90%, 91%, 92%, 93%, 94%, 95%, 96%, 97%, 98%, or 99% identical to SEQ ID NO: 409. In some embodiments, an anti-TL1A antibody described herein comprises a Fc region comprising SEQ ID NO: 410 or a sequence at least about 90%, 91%, 92%, 93%, 94%, 95%, 96%, 97%, 98%, or 99% identical to SEQ ID NO: 410. In some embodiments, an anti-TL1A antibody described herein comprises a Fc region comprising SEQ ID NO: 411 or a sequence at least about 90%, 91%, 92%, 93%, 94%, 95%, 96%, 97%, 98%, or 99% identical to SEQ ID NO: 411. In some embodiments, an anti-TL1A antibody described herein comprises a Fc region comprising SEQ ID NO: 412 or a sequence at least about 90%, 91%, 92%, 93%, 94%, 95%, 96%, 97%, 98%, or 99% identical to SEQ ID NO: 412. In some embodiments, an anti-TL1A antibody described herein comprises a Fc region comprising SEQ ID NO: 413 or a sequence at least about 90%, 91%, 92%, 93%, 94%, 95%, 96%, 97%, 98%, or 99% identical to SEQ ID NO: 413.

By way of further example, in certain embodiments, an anti-TL1A antibody described herein comprises a heavy chain comprising SEQ ID NO: 501 or a sequence at least about 90%, 91%, 92%, 93%, 94%, 95%, 96%, 97%, 98%, or 99% identical to SEQ ID NO: 501. In certain embodiments, an anti-TL1A antibody described herein comprises a heavy chain comprising SEQ ID NO: 502 or a sequence at least about 90%, 91%, 92%, 93%, 94%, 95%, 96%, 97%, 98%, or 99% identical to SEQ ID NO: 502. In certain embodiments, an anti-TL1A antibody described herein comprises a heavy chain comprising SEQ ID NO: 503 or a sequence at least about 90%, 91%, 92%, 93%, 94%, 95%, 96%, 97%, 98%, or 99% identical to SEQ ID NO: 503. In certain embodiments, an anti-TL1A antibody described herein comprises a heavy chain comprising SEQ ID NO: 504 or a sequence at least about 90%, 91%, 92%, 93%, 94%, 95%, 96%, 97%, 98%, or 99% identical to SEQ ID NO: 504. In certain embodiments, an anti-TL1A antibody described herein comprises a heavy chain comprising SEQ ID NO: 505 or a sequence at least about 90%, 91%, 92%, 93%, 94%, 95%, 96%, 97%, 98%, or 99% identical to SEQ ID NO: 505. In certain embodiments, an anti-TL1A antibody described herein comprises a heavy chain comprising SEQ ID NO: 506 or a sequence at least about 90%, 91%, 92%, 93%, 94%, 95%, 96%, 97%, 98%, or 99% identical to SEQ ID NO: 506. In certain embodiments, an anti-TL1A antibody described herein comprises a heavy chain comprising SEQ ID NO: 507 or a sequence at least about 90%, 91%, 92%, 93%, 94%, 95%, 96%, 97%, 98%, or 99% identical to SEQ ID NO: 507. In certain embodiments, an anti-TL1A antibody described herein comprises a heavy chain comprising SEQ ID NO: 508 or a sequence at least about 90%, 91%, 92%, 93%, 94%, 95%, 96%, 97%, 98%, or 99% identical to SEQ ID NO: 508. In certain embodiments, an anti-TL1A antibody described herein comprises a heavy chain comprising SEQ ID NO: 509 or a sequence at least about 90%, 91%, 92%, 93%, 94%, 95%, 96%, 97%, 98%, or 99% identical to SEQ ID NO: 509. In certain embodiments, an anti-TL1A antibody described herein comprises a heavy chain comprising SEQ ID NO: 510 or a sequence at least about 90%, 91%, 92%, 93%, 94%, 95%, 96%, 97%, 98%, or 99% identical to SEQ ID NO: 510. In certain embodiments, an anti-TL1A antibody described herein comprises a heavy chain comprising SEQ ID NO: 511 or a sequence at least about 90%, 91%, 92%, 93%, 94%, 95%, 96%, 97%, 98%, or 99% identical to SEQ ID NO: 511. In certain embodiments, an anti-TL1A antibody described herein comprises a heavy chain comprising SEQ ID NO: 512 or a sequence at least about 90%, 91%, 92%, 93%, 94%, 95%, 96%, 97%, 98%, or 99% identical to SEQ ID NO: 512. In certain embodiments, an anti-TL1A antibody described herein comprises a heavy chain comprising SEQ ID NO: 513 or a sequence at least about 90% 91%, 92%, 93%, 94%, 95%, 96%, 97%, 98%, or 99% identical to SEQ ID NO. 513. In certain embodiments, the heavy chain is paired with a light chain comprising SEQ ID NO: 514 or a sequence at least about 90%, 91%, 92%, 93%, 94%, 95%, 96%, 97%, 98%, or 99% identical to SEQ ID NO: 514. In certain embodiments, the heavy chain is paired with the light chain variable region of SEQ ID NO: 514 or a sequence at least about 90%, 91%, 92%, 93%, 94%, 95%, 96%, 97%, 98%, or 99% identical to the light chain variable region of SEQ ID NO: 514. In certain embodiments, the heavy chain is paired with the light chain variable region of SEQ ID NO: 514 or a sequence at least about 90%, 91%, 92%, 93%, 94%, 95%, 96%, 97%, 98%, or 99% identical to the light chain variable region of SEQ ID NO: 515.

In some embodiments, anti-TL1A described herein comprise a light chain constant region comprising SEQ ID NO: 319 or a sequence at least about 90%, 91%, 92%, 93%, 94%, 95%, 96%, 97%, 98%, or 99% identical to SEQ ID NO: 319.

### Further Non-Limiting Exemplary Anti-TL1A Antibodies

In one aspect, provided herein is a first embodiment of an anti-TL1A antibody comprising a heavy chain comprising a HCDR1, a HCDR2, and a HCDR3, and a light chain comprising a LCDR1, a LCDR2, and a LCDR3. Non-limiting additional embodiments include: (Embodiment 2) The anti-TL1A antibody of embodiment 1, comprising a HCDR1 comprising SEQ ID NO: 1. (Embodiment 3) The anti-TL1A antibody of embodiment 1 or embodiment 2, comprising a HCDR2 comprising SEQ ID NO: 2. (Embodiment 4) The anti-TL1A antibody of embodiment 1 or embodiment 2, comprising a HCDR2 comprising SEQ ID NO: 3. (Embodiment 5) The anti-TL1A antibody of embodiment I or embodiment 2, comprising a HCDR2 comprising SEQ ID NO: 4. (Embodiment 6) The anti-TL1A antibody of embodiment 1 or embodiment 2, comprising a HCDR2 comprising SEQ ID NO: 5. (Embodiment 7) The anti-TL1A antibody of any one of embodiments 1-6, comprising a HCDR3 comprising SEQ ID NO: 6. (Embodiment 8) The anti-TL1A antibody of any one of embodiments 1-6, comprising a HCDR3 comprising SEQ ID NO: 7. (Embodiment 9) The anti-TL1A antibody of any one of embodiments 1-6, comprising a HCDR3 comprising SEQ ID NO: 8. (Embodiment 10) The anti-TL1A antibody of any one of embodiments 1-6, comprising a HCDR3 comprising SEQ ID NO. 9. (Embodiment 11) The anti-TL1A antibody of any one of embodiments 1-10, comprising a LCDR1 comprising SEQ ID NO: 10. (Embodiment 12) The anti-TL1A antibody of any one of embodiments 1-11, comprising a LCDR2 comprising SEQ ID NO: 11. (Embodiment 13) The anti-TL1A antibody of any one of embodiments 1-12, comprising a LCDR3 comprising SEQ ID NO: 12. (Embodiment 14) The anti-TL1A antibody of any one of embodiments 1-12, comprising a LCDR3 comprising SEQ ID NO: 13. (Embodiment 15) The anti-TL1A antibody of any one of embodiments 1-12, comprising a LCDR3 comprising SEQ ID NO: 14 or 15.

(Embodiment 16) The anti-TL1A antibody of any one of embodiments 1-15, comprising a heavy chain framework comprising IGHV1-46*02. (Embodiment 17) The anti-TL1A antibody of any one of embodiments 1-15, comprising a heavy chain framework comprising a variant of IGHV1-46*02 comprising between about 1 and about 20 amino acid substitutions from SEQ ID NO: 316. (Embodiment 18) The anti-TL1A antibody of any one of embodiments 1-15, comprising a heavy chain framework comprising a variant of IGHV1-46*02 comprising between about 1 and about 9 amino acid substitutions from SEQ ID NO: 316. (Embodiment 19) The anti-TL1A antibody of any one of embodiments 1-15, comprising a heavy chain framework comprising a variant of IGHV 1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. (Embodiment 20) The anti-TL1A antibody of any one of embodiments 17-19, wherein the heavy chain framework substitution comprises Q1E, as determined by Aho or Kabat numbering. (Embodiment 21) The anti-TL1A antibody of any one of embodiments 17-20, wherein the heavy chain framework substitution comprises R45K, as determined by Aho or Kabat numbering. (Embodiment 22) The anti-TL1A antibody of any one of embodiments 17-21, wherein the heavy chain framework substitution comprises A47R, as determined by Aho or Kabat numbering. (Embodiment 23) The anti-TL1A antibody of any one of embodiments 17-22, wherein the heavy chain framework substitution comprises M551, as determined by Aho or Kabat numbering. (Embodiment 24) The anti-TL1A antibody of any one of embodiments 17-23, wherein the heavy chain framework substitution comprises V78A, as determined by Aho or Kabat numbering. (Embodiment 25) The anti-TL1A antibody of any one of embodiments 17-24, wherein the heavy chain framework substitution comprises M80I, as determined by Aho or Kabat numbering. (Embodiment 26) The anti-TL1A antibody of any one of embodiments 17-25, wherein the heavy chain framework substitution comprises R82T, as determined by Aho or Kabat numbering. (Embodiment 27) The anti-TL1A antibody of any one of embodiments 17-26, wherein the heavy chain framework substitution comprises V89A, as determined by Aho or Kabat numbering. (Embodiment 28) The anti-TL1A antibody of any one of embodiments 17-27, wherein the heavy chain framework substitution comprises M91L, as determined by Aho or Kabat numbering.

(Embodiment 29) The anti-TL1A antibody of any one of embodiments 1-15, comprising a heavy chain framework comprising SEQ ID NO: 301. (Embodiment 30) The anti-TL1A antibody of embodiment 29, wherein X1 is Q. (Embodiment 31) The anti-TL1A of embodiment 29, wherein X1=E. (Embodiment 32) The anti-TL1A of any one of embodiments 29-31, wherein X2=R. (Embodiment 33) The anti-TL1A of any one of embodiments 29-31, wherein X2=K. (Embodiment 34) The anti-TL1A of any one of embodiments 29-33, wherein X3=A. (Embodiment 35) The anti-TL1A of any one of embodiments 29-33, wherein X3=R. (Embodiment 36) The anti-TL1A of any one of embodiments 29-35, wherein X4=M. (Embodiment 37) The anti-TL1A of any one of embodiments 29-35, wherein X4=I. (Embodiment 38) The anti-TL1A of any one of embodiments 29-37, wherein X5=V. (Embodiment 39) The anti-TL1A of any one of embodiments 29-37, wherein X5=A. (Embodiment 40) The anti-TL1A of any one of embodiments 29-39, wherein X6=M. (Embodiment 41) The anti-TL1A of any one of embodiments 29-39, wherein X6=I. (Embodiment 42) The anti-TL1A of any one of embodiments 29-41, wherein X7=R. (Embodiment 43) The anti-TL1A of any one of embodiments 29-41, wherein X7=T. (Embodiment 44) The anti-TL1A of any one of embodiments 29-43, wherein X8=V. (Embodiment 45) The anti-TL1A of any one of embodiments 29-43, wherein X8=A. (Embodiment 46) The anti-TL1A of any one of embodiments 29-45, wherein X9=M. (Embodiment 47) The anti-TL1A of any one of embodiments 29-45, wherein X9=L.

(Embodiment 48) The anti-TL1A antibody of any one of embodiments 1-15, comprising a heavy chain framework comprising SEQ ID NO: 302. (Embodiment 49) The anti-TL1A antibody of embodiment 48, wherein X1 is Q. (Embodiment 50) The anti-TL1A of embodiment 48, wherein X1=E. (Embodiment 51) The anti-TL1A of any one of embodiments 48-50, wherein X2=R. (Embodiment 52) The anti-TL1A of any one of embodiments 48-50, wherein X2=K. (Embodiment 53) The anti-TL1A of any one of embodiments 48-52, wherein X3=A. (Embodiment 54) The anti-TL1A of any one of embodiments 48-52, wherein X3=R. (Embodiment 55) The anti-TL1A of any one of embodiments 48-54, wherein X4=M. (Embodiment 56) The anti-TL1A of any one of embodiments 48-54, wherein X4=I. (Embodiment 57) The anti-TL1A of any one of embodiments 48-56, wherein X5=V. (Embodiment 58) The anti-TL1A of any one of embodiments 48-56, wherein X5=A. (Embodiment 59) The anti-TL1A of any one of embodiments 48-58, wherein X6=M. (Embodiment 60) The anti-TL1A of any one of embodiments 48-58, wherein X6=1. (Embodiment 61) The anti-TL1A of any one of embodiments 48-60, wherein X7=R. (Embodiment 62) The anti-TL1A of any one of embodiments 48-60, wherein X7=T. (Embodiment 63) The anti-TL1A of any one of embodiments 48-62, wherein X8=V. (Embodiment 64) The anti-TL1A of any one of embodiments 48-62, wherein X8=A. (Embodiment 65) The anti-TL1A of any one of embodiments 48-64, wherein X9=M. (Embodiment 66) The anti-TL1A of any one of embodiments 48-64, wherein X9=L.

(Embodiment 67) The anti-TL1A antibody of any one of embodiments 1-66, comprising a light chain framework comprising IGKV3-20*01. (Embodiment 68) The anti-TL1A antibody of any one of embodiments 1-66, comprising a light chain framework comprising a variant of IGKV3-20*01 comprising between about 1 and about 20 amino acid substitutions from SEQ ID NO: 317. (Embodiment 69) The anti-TL1A antibody of any one of embodiments 1-66, comprising a light chain framework comprising a variant of IGKV3-20*01 comprising about 1 amino acid substitution from SEQ ID NO: 317. (Embodiment 70) The anti-TL1A antibody of any one of embodiments 1-66, comprising a light chain framework comprising a variant of IGKV3-20*01 comprising about 2 amino acid substitutions from SEQ ID NO: 317. (Embodiment 71) The anti-TL1A antibody of any one of embodiments 1-66, comprising a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. (Embodiment 72) The anti-TL1A antibody of any one of embodiments 69-71, wherein the light chain framework substitution comprises Q1E, as determined by Aho or Kabat numbering. (Embodiment 73) The anti-TL1A antibody of any one of embodiments 69-72, wherein the light chain framework substitution comprises R45K, as determined by Aho or Kabat numbering.

(Embodiment 74) The anti-TL1A antibody of any one of embodiments 1-66, comprising a light chain comprising a light chain framework comprising SEQ ID NO: 303. (Embodiment 75) The anti-TL1A antibody of embodiment 74, wherein X10 is L. (Embodiment 76) The anti-TL1A antibody of embodiment 74, wherein X10 is P. (Embodiment 77) The anti-TL1A antibody of any one of embodiments 74-76, wherein X11 is L. (Embodiment 78) The anti-TL1A antibody of any one of embodiments 74-76, wherein X11 is W.

(Embodiment 79) The anti-TL1A of any one of embodiments 1-78, comprising a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 3318, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S. (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ce) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P, (nn) 385P, (00) 424H, 424M, or 424V, (pp) 4341, (qq) 438G. (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P33IS (IgGlo), (ccc) L234A, L235A, and P329A, (ddd) G236R and L328R, (cee) G237A, (fff) F241A, (ggg) V264A, (hhh) D265A, (iii) D265A and N297A, (jjj) D265A and N297G. (kkk) D270A, (III) A330L, (mmm) P331A or P331S, or (nnn) any combination of (a)-(uu), per Kabat numbering. (Embodiment 80) The anti-TL1A of any one of embodiments 1-78, comprising a (i) human IgG4 Fc region or (ii) a human IgG4 Fc region comprising (a) S228P and L235E, or (b) S228P. F234A, and L235A, per Kabat numbering. (Embodiment 81) The anti-TL1A of any one of embodiments 1-78, comprising a human IgG2 Fc region; IgG2-IgG4 cross-subclass Fc region; IgG2-IgG3 cross-subclass Fe region; IgG2 comprising H268Q, V309L, A330S, P331S (IgG2m4); or IgG2 comprising V234A, G237A. P238S, H268A, V309L, A330S, P33IS (IgG2 (Embodiment 82) The anti-TL1A of any one of embodiments 1-81, comprising a heavy chain Fc region comprising any one of SEQ ID NOS: 320-362. (Embodiment 83) The anti-TL1A antibody of any one of embodiments 1-82, comprising a light chain constant region comprising SEQ ID NO: 319.

(Embodiment 84) The anti-TL1A antibody of any one of embodiments 1-83, comprising at least about 80% monomeric fraction as determined by the size exclusion chromatography method described herein. (Embodiment 85) The anti-TL1A antibody of any one of embodiments 1-84, comprising at least about 81%, at least about 82%, at least about 83%, or at least about 84% monomeric fraction as determined by the size exclusion chromatography method described herein. (Embodiment 86) The anti-TL1A antibody of any one of embodiments 1-85, comprising at least about 85% monomeric fraction as determined by the size exclusion chromatography method described herein. (Embodiment 87) The anti-TL1A antibody of any one of embodiments 1-86, comprising at least about 86%, at least about 87%, at least about 88%, or at least about 89% monomeric fraction as determined by the size exclusion chromatography method described herein. (Embodiment 88) The anti-TL1A antibody of any one of embodiments 1-87, comprising at least about 90% monomeric fraction as determined by the size exclusion chromatography method described herein. (Embodiment 89) The anti-TL1A antibody of any one of embodiments 1-88, comprising at least about 91%, at least about 92%, at least about 93%, or at least about 94% monomeric fraction as determined by the size exclusion chromatography method described herein. (Embodiment 90) The anti-TL1A antibody of any one of embodiments 1-89, comprising at least about 95% monomeric fraction as determined by the size exclusion chromatography method described herein. (Embodiment 91) The anti-TL1A antibody of any one of embodiments 1-90, comprising at least about 96%, at least about 97%, at least about 98%, or at least about 99% monomeric fraction as determined by the size exclusion chromatography method described herein.

(Embodiment 92) The anti-TL1A antibody of any one of embodiments 1-91, comprising at least about 2 μg/mL expression as determined by the method disclosed herein. (Embodiment 93) The anti-TL1A antibody of any one of embodiments 1-92, comprising between about 2 μg/mL and about 60 μg/mL expression as determined by the method disclosed herein. (Embodiment 94) The anti-TL1A antibody of any one of embodiments 1-93, comprising between about 5 μg/mL and about 60 g/mL expression as determined by the method disclosed herein. (Embodiment 95) The anti-TL1A antibody of any one of embodiments 1-94, comprising between about 10 μg/mL and about 60 μg/mL expression as determined by the method disclosed herein. (Embodiment 96) The anti-TL1A antibody of any one of embodiments 1-95, comprising at least about 5 μg/mL expression as determined by the method disclosed herein. (Embodiment 97) The anti-TL1A antibody of any one of embodiments 1-96, comprising at least about 10 μg/mL expression as determined by the method disclosed herein. (Embodiment 98) The anti-TL1A antibody of any one of embodiments 1-97, comprising at least about 15 μg/mL expression as determined by the method disclosed herein. (Embodiment 99) The anti-TL LA antibody of any one of embodiments 1-98, comprising at least about 20 μg/mL expression as determined by the method disclosed herein. (Embodiment 100) The anti-TL1A antibody of any one of embodiments 1-91, comprising between about 2 μg/mL and about 50 μg/mL, between about 2 μg/mL and about 40 μg/mL, between about 2 μg/mL and about 30 μg/mL expression, between about 2 μg/mL and about 20 μg/mL, between about 5 μg/mL and about 50 g/mL, between about 5 μg/mL and about 40 μg/mL, between about 5 μg/mL and about 30 g/mL, between about 10 μg/mL and about 50 μg/mL, between about 10 μg/mL and about 40 μg/mL, or between about 10 μg/mL and about 30 μg/mL as determined by the method disclosed herein. (Embodiment 101) The anti-TL1A antibody of any one of embodiments 1-91, comprising about 2, 3, 4, 5, 6, 7, 8, 9, 10, 11, 12, 13, 14, 15, 16, 17, 18, 19, 20, 21, 22, 23, 24, 25, 26, 27, 28, 29, 30 g/mL expression as determined by the method disclosed herein.

In some embodiments, an anti-TL1A antibody comprises antibody A. As used herein, antibody A comprises the CDRs of antibody A in Table 20. In some cases, antibody A comprises a heavy chain framework comprising SEQ ID NO: 301 (Embodiment XIVQLVQSGAEVKKPGASVKVSCKAS[HCDR1]WVX2QX3PGQGLEWX4G[HCDR2]RX5 TX6TX7DTSTSTX8YX9ELSSLRSEDTAVYYCAR[HCDR3]WGQGTTVTVSS), wherein X1=Q or E, X2=R or K. X3=A or R, X4=M or I, X5=V or A, X6=M or I, X7=R or T, X8=V or A, and X9=M or L. In some cases, antibody A comprises a light chain framework comprising SEQ ID NO: 303 (Embodiment EIVLTQSPGTLSLSPGERATLSC[LCDR1]WYQQKPGQAPRX10X11IY [LCDR2]GIPDRESG SGSGTDFTLTISRLEPEDFAVYYC[LCDR3]FGGGTKLEIK), wherein X10=L or P and X11=L or W. In some cases, antibody A comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A comprises a constant region comprising reduced ADCC and/or CDC as compared to IgG1. For instance, antibody A comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 31IS, (cc) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P, (nn) 385P, (00) 424H, 424M, or 424V, (pp) 4341, (qq) 438G, (rr) 439E, 439H, or 439Q, (ss) 440A, 440D, 440E, 440F, 440M, 440T, or 440V. (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R. (ece) G237A, (fff) F241A. (ggg) V264A, (hbb) D265A. (iii) D265A and N297A, (jjj) D265A and N297G, (kkk) D270A, (III) A330L, (mmm) P331A or P331S, or (nnn) any combination of (a)-(uu), per Kabat numbering. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody A comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody A is expressed from FreeStyle 293-F (e.g., ThermoFisher Scientific #R79007) cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody A is expressed from FreeStyle 293-F cells at an expression level of between about 2 μg/mL to about 60 μg/mL. In some cases, antibody A is expressed from FreeStyle 293-F cells at an expression level of between about 10 μg/mL to about 60 μg/mL. In some cases, antibody A comprises a viscosity less than about 30 mPa-s. In some cases, antibody A comprises a viscosity from about 4 mPa-s to about 30 mPa-s, or about 4, 5, 6, 7, 8, 9, 10, 11, 12, 13, 14, 15, 16, 17, 18, 19, 20, 21, 22, 23, 24, 25, 26, 27, 28, 29, 30 mPa-s. In some cases, antibody A is formulated in a solution having a concentration of about 10 mg/ml to about 170 mg/ml, with a viscosity less than about 30 mPa-s. In some cases, the formulation has a pH of about 5 to about 7.5.

In some embodiments, an anti-TL1A antibody comprises antibody B. As used herein, antibody B comprises the CDRs of antibody B in Table 20. In some cases, antibody B comprises a heavy chain framework comprising SEQ ID NO: 301 (XIVQLVQSGAEVKKPGASVKVSCKAS[HCDR1]WVX2QX3PGQGLEWX4G[HCDR2]RX STX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T. X8=V or A, and X9=M or L. In some cases, antibody B comprises a light chain framework comprising SEQ ID NO: 303 (EIVLTQSPGTLSLSPGERATLSC[LCDR1]WYQQKPGQAPRX10XIIIY[LCDR2]GIPDRFS GSGSGTDFTLTISRLEPEDFAVYYC[LCDR3]FGGGTKLEIK), wherein X10=L or P and X11=L or W. In some cases, antibody B comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B comprises a constant region comprising reduced ADCC and/or CDC as compared to IgG1. For instance, antibody B comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T. (j) 329A, 329G, 329Y, or 329R (k) 331S, (1) 236F or 236R, (m) 238A, 238E, 238G, 238H, 238I, 238V, 238 W, or 238Y, (n) 248A. (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 27IT, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, Gj) 373A, 373G, or 373S, (kk) 376E, 376 W, or 376Y, (II) 380D, (mm) 382D or 382P, (nn) 385P, (00) 424H, 424M, or 424V, (pp) 4341, (qq) 438G. (rr) 439E, 439H, or 439Q. (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody B comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody B is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody B is expressed from FreeStyle 293-F cells at an expression level of between about 2 μg/mL to about 60 μg/mL. In some cases, antibody B is expressed from FreeStyle 293-F cells at an expression level of between about 10 μg/mL to about 60 μg/mL.

In some embodiments, an anti-TL1A antibody comprises antibody C. As used herein, antibody C comprises the CDRs of antibody C in Table 20. In some cases, antibody C comprises a heavy chain framework comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody C comprises a light chain framework comprising SEQ ID NO: 303 (EIVLTQSPGTLSLSPGERATLSC[LCDR1]WYQQKPGQAPRX10X11TY[LCDR2]GIPDRES GSGSGTDFTLTISRLEPEDFAVYYC[LCDR3]FGGGTKLEIK), wherein X10=L or P and X11=L or W. In some cases, antibody C comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C comprises a constant region comprising reduced ADCC and/or CDC as compared to IgG1. For instance, antibody C comprises a human IgG1 Fc region comprising (a) 297A, 297Q, 297G, or 297D, (b) 279F, 279K, or 279L, (c) 228P. (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (1) 329A, 329G, 329Y, or 329R (k) 331S, (1) 236F or 236R, (m) 238A, 238E, 238G, 238H, 238I, 238V, 238 W, or 238Y, (n) 248A, (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (j) 373A, 373G, or 373S, (kk) 376E, 376 W, or 376Y, (II) 380D, (mm) 382D or 382P, (nn) 385P, (00) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (VV) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (eee) G237A, (fff) F241A, (ggg) V264A. (hhh) D265A, (iii) D265A and N297A, (ij) D265A and N297G, (kkk) D270A, (III) A330L, (mmm) P331A or P331S, or (nnn) any combination of (a)-(uu), per Kabat numbering. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody C comprises a constant region comprising at least about 85%, 87%, 88%, 89%, 90%, 91%, 92%, 93%, 94%, 95%, 96%, 97%), 98%, 99%, or 100% identity to SEQ ID NO: 327. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 346, In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody C comprises a constant region comprising at least about 85%, 87%, 88%, 89%, 90%, 91%, 92%, 93%, 94%, 95%, 96%, 97%), 98%, 99%, or 100% identity to SEQ ID NO: 351. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody C comprises a constant region comprising at least about 85%, 87%, 88%, 89% 90%, 91%, 92%, 93%, 94%, 95%, 96%, 97%, 98%, 99%, or 100% identity to SEQ ID NO: 357. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody C comprises a constant region comprising at least about 85%, 87%, 88%, 89%, 90%, 91%, 92%, 93%, 94%, 95%, 96%, 97%), 98%, 99%, or 100% identity to SEQ ID NO: 359. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody C comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody C is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody C is expressed from FreeStyle 293-F cells at an expression level of between about 2 μg/mL, to about 60 μg/mL. In some cases, antibody C is expressed from FreeStyle 293-F cells at an expression level of between about 10 g/mL to about 60 μg/mL.

In some embodiments, an anti-TL1A antibody comprises antibody D. As used herein, antibody D comprises the CDRs of antibody D in Table 20. In some cases, antibody D comprises a heavy chain framework comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E. X2=R or K, X3=A or R, X4=M or I. X5=V or A, X6=M or 1, X7=R or T, X8=V or A. and X9=M or L. In some cases, antibody D comprises a light chain framework comprising SEQ ID NO: 303 (EIVLTQSPGTLSLSPGERATLSC[LCDR1]WYQQKPGQAPRX10X11IY [LCDR2]GIPDRFS GSGSGTDFTLTISRLEPEDFAVYYC[LCDR3]FGGGTKLEIK), wherein X10=L or P and X11=L or W. In some cases, antibody D comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D comprises a constant region comprising reduced ADCC and/or CDC as compared to IgG1. For instance, antibody D comprises a human IgG1 Fc region comprising (a) 297A, 297Q, 297G, or 297D, (b) 279F, 279K, or 279L. (c) 228P. (d) 235A, 235E, 235G, 235Q, 235R, or 235S, (e) 237A, 237E, 237K, 237N, or 237R, (f) 234A, 234V, or 234F, (g) 233P, (h) 328A, (1) 327Q or 327T, (1) 329A, 329G, 329Y, or 329R (k) 331S, (1) 236F or 236R, (m) 238A, 238E, 238G, 238H, 238I, 238V, 238 W, or 238Y, (n) 248A, (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q. (w) 270A, 270G, 270M, or 270N, (x) 27IT, (y) 272N, (z) 292E, 292F, 292G, or 2921, (aa) 293S, (bb) 301 W. (cc) 304E, (dd) 311E, 311G, or 311S, (ee) 316F, (ff) 328V, (gg) 330R, (hh) 339E or 339L, (ii) 3431 or 343V, (ij) 373A, 373G, or 373S, (kk) 376E, 376 W, or 376Y, (II) 380D, (mm) 382D or 382P, (nn) 385P, (00) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A. (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R. (eee) G237A, (fff) F241A, (ggg) V264A, (hhh) D265A, (iii) D265A and N297A, (jj) D265A and N297G, (kkk) D270A, (III) A330L, (mmm) P331A or P331S, or (nnn) any combination of (a)-(uu), per Kabat numbering. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody D comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody D is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody D is expressed from FreeStyle 293-F cells at an expression level of between about 2 μg/mL to about 60 μg/mL. In some cases, antibody D is expressed from FreeStyle 293-F cells at an expression level of between about 10 μg/mL to about 60 μg/mL.

In some embodiments, an anti-TL1A antibody comprises antibody E. As used herein, antibody E comprises the CDRs of antibody E in Table 20. In some cases, antibody E comprises a heavy chain framework comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E. X2=R or K, X3=A or R, X4=M or I, X5=V or A, X6=M or I, X7=R or T, X8=V or A. and X9=M or L. In some cases, antibody E comprises a light chain framework comprising SEQ ID NO: 303 (EIVLTQSPGTLSLSPGERATLSC[LCDR1]WYQQKPGQAPRX10X11IY[LCDR2]GIPDRES GSGSGTDFTLTISRLEPEDFAVYYC[LCDR3]FGGGTKLEIK), wherein X10=L or P and X11=L or W. In some cases, antibody E comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E comprises a constant region comprising reduced ADCC and/or CDC as compared to IgG1. For instance, antibody E comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, ( ) 329A, 329G, 329Y, or 329R (k) 331S, (1) 236F or 236R, (m) 238A, 238E, 238G, 238H, 238I, 238V, 238 W, or 238Y, (n) 248A. (0) 254D, 254E, 254G, 254H, 254I, 254N, 254P, 254Q, 254T, or 254V. (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301 W, (cc) 304E, (dd) 311E, 311G, or 311S, (ce) 316F, (ff) 328V, (gg) 330R, (bh) 339E or 339L, (ii) 3431 or 343V, (j) 373A, 373G, or 373S, (kk) 376E, 376 W, or 376Y, (II) 380D. (mm) 382D or 382P, (nn) 385P, (00) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P33IS (bbb) L234A, L235A, G237A, P238S, H268A, A330S, and P331S (IgGlo), (ccc) L234A, L235A, and P329A, (ddd) G236R and L328R, (cee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody E comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody E is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody E is expressed from FreeStyle 293-F cells at an expression level of between about 2 μg/mL to about 60 μg/mL. In some cases, antibody E is expressed from FreeStyle 293-F cells at an expression level of between about 10 μg/mL to about 60 μg/mL.

In some embodiments, an anti-TL1A antibody comprises antibody F. As used herein, antibody F comprises the CDRs of antibody F in Table 20. In some cases, antibody F comprises a heavy chain framework comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or B, X2=R or K, X3=A or R, X4=M or I, X5=V or A, X6=M or I, X7=R or T, X8=V or A, and X9=M or L. In some cases, antibody F comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC|LCDR3|FGGGTKLEIK), wherein X10=L or P and X11=L or W. In some cases, antibody F comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F comprises a constant region comprising reduced ADCC and/or CDC as compared to IgG1. For instance, antibody F comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T. (j) 329A, 329G, 329Y, or 329R (k) 331S. (1) 236F or 236R, (m) 238A, 238E, 238G, 238H, 2381, 238V, 238 W, or 238Y, (n) 248A, (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 27IT, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376B, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody F comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody F is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 g/mL as determined by the method described in Example 2. In some cases, antibody F is expressed from FreeStyle 293-F cells at an expression level of between about 2 μg/mL to about 60 μg/mL. In some cases, antibody Fis expressed from FreeStyle 293-F cells at an expression level of between about 10 μg/mL to about 60 μg/mL.

In some embodiments, an anti-TL1A antibody comprises antibody G. As used herein, antibody G comprises the CDRs of antibody G in Table 20. In some cases, antibody G comprises a heavy chain framework comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody G comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody G comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G comprises a constant region comprising reduced ADCC and/or CDC as compared to IgG1. For instance, antibody G comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R. (m) 238A, 238E, 238G, 238H, 2381, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 27IT, (y) 272N, (z) 292E, 292F, 292G, or 292I, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (j) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (eee) G237A, (fff) F241A, (ggg) V264A, (hhh) D265A, (iii) D265A and N297A, (jj) D265A and N297G, (kkk) D270A. (III) A330L, (mmm) P331A or P331S, or (nnn) any combination of (a)-(uu), per Kabat numbering. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody G comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody G is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, SS, 5, 57, 58, 59 or 60 μg/mL as determined by the method described in Example 2. In some cases, antibody G is expressed from FreeStyle 293-F cells at an expression level of between about 2 μg/mL to about 60 μg/mL. In some cases, antibody G is expressed from FreeStyle 293-F cells at an expression level of between about 10 μg/mL to about 60 μg/mL.

In some embodiments, an anti-TL1A antibody comprises antibody H. As used herein, antibody H comprises the CDRs of antibody H in Table 20. In some cases, antibody H comprises a heavy chain framework comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A. X6=M or I, X7=R or T, X8=V or A, and X9=M or L. In some cases, antibody H comprises a light chain framework comprising SEQ ID NO: 303 (EIVLTQSPGTLSLSPGERATLSC[LCDR1]WYQQKPGQAPRX10X11IY[LCDR2]GIPDRES GSGSGTDFTLTISRLEPEDFAVYYC[LCDR3]FGGGTKLEIK), wherein X10=L or P and X11=L or W. In some cases, antibody H comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H comprises a constant region comprising reduced ADCC and/or CDC as compared to IgG1. For instance, antibody H comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q. (w) 270A, 270G, 270M, or 270N, (x) 27IT, (y) 272N, (7) 292E, 292F, 292G, or 2921, (aa) 293S, (bb) 301 W. (cc) 304E, (dd) 311E, 311G, or 311S, (ee) 316F, (ff) 328V, (gg) 330R, (hh) 339E or 339L, (ii) 3431 or 343V, (ij) 373A, 373G, or 373S, (kk) 376E, 376 W, or 376Y, (II) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P. (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R. (eee) G237A, (fff) F241A. (ggg) V264A, (hhh) D265A, (iii) D265A and N297A, (jj) D265A and N297G, (kkk) D270A, (III) A330L, (mmm) P331A or P331S, or (nnn) any combination of (a)-(uu), per Kabat numbering. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody H comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody H is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody H is expressed from FreeStyle 293-F cells at an expression level of between about 2 g/mL to about 60 μg/mL. In some cases, antibody H is expressed from FreeStyle 293-F cells at an expression level of between about 10 μg/mL to about 60 μg/mL.

In some embodiments, an anti-TL1A antibody comprises antibody I. As used herein, antibody I comprises the CDRs of antibody I in Table 20. In some cases, antibody I comprises a heavy chain framework comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody I comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody I comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody I comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody I comprises a constant region comprising reduced ADCC and/or CDC as compared to IgG1. For instance, antibody I comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V. (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 27IT, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, Gj) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V. (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A. (ddd) G236R and L328R. (eee) G237A, (fff) F241A. (ggg) V264A, (hhh) D265A, (iii) D265A and N297A, (jj) D265A and N297G, (kkk) D270A, (III) A330L, (mmm) P331A or P331S, or (nnn) any combination of (a)-(uu), per Kabat numbering. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody I comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody I is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody I is expressed from FreeStyle 293-F cells at an expression level of between about 2 μg/mL to about 60 g/mL. In some cases, antibody I is expressed from FreeStyle 293-F cells at an expression level of between about 10 μg/mL to about 60 μg/mL.

In some embodiments, an anti-TL1A antibody comprises antibody A2. As used herein, antibody A2 comprises the CDRs of antibody A2 in Table 20. In some cases, antibody A2 comprises a heavy chain framework comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody A2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody A2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A2 comprises a constant region comprising reduced ADCC and/or CDC as compared to IgG1. For instance, antibody A2 comprises a human IgG1 Fc region comprising (a) 297A, 297Q, 297G, or 297D, (b) 279F, 279K, or 279L, (c) 228P, (d) 235A, 23SE, 235G, 235Q, 235R, or 235S, (e) 237A, 237E, 237K, 237N, or 237R, (f) 234A, 234V, or 234F, (g) 233P. (h) 328A, (i) 327Q or 327T, (1) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q. (w) 270A, 270G, 270M, or 270N, (x) 27IT, (y) 272N, (z) 292E, 292F, 292G, or 2921, (aa) 293S, (bb) 301 W, (cc) 304E, (dd) 311E, 311G, or 311S, (ce) 316F, (ff) 328V, (gg) 330R, (bh) 339E or 339L, (ii) 3431 or 343V, (jj) 373A, 373G, or 373S, (kk) 376E, 376 W, or 376Y, (II) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (eee) G237A. (fff) F241A, (ggg) V264A, (hhh) D265A, (iii) D265A and N297A, (jj) D265A and N297G, (kkk) D270A, (III) A330L, (mmm) P331A or P331S, or (nnn) any combination of (a)-(uu), per Kabat numbering. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody A2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody A2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 g/mL as determined by the method described in Example 2. In some cases, antibody A2 is expressed from FreeStyle 293-F cells at an expression level of between about 2 μg/mL to about 60 μg/mL. In some cases, antibody A2 is expressed from FreeStyle 293-F cells at an expression level of between about 10 μg/mL to about 60 μg/mL.

In some embodiments, an anti-TL1A antibody comprises antibody B2. As used herein, antibody B2 comprises the CDRs of antibody B2 in Table 20. In some cases, antibody B2 comprises a heavy chain framework comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I. X5=V or A, X6=M or I, X7=R or T, X8=V or A, and X9=M or L. In some cases, antibody B2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody B2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B2 comprises a constant region comprising reduced ADCC and/or CDC as compared to IgG1. For instance, antibody B2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (1) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, Gj) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q. (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody B2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody B2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody B2 is expressed from FreeStyle 293-F cells at an expression level of between about 2 μg/mL to about 60 μg/mL. In some cases, antibody B2 is expressed from FreeStyle 293-F cells at an expression level of between about 10 μg/mL to about 60 μg/mL.

In some embodiments, an anti-TL1A antibody comprises antibody C2. As used herein, antibody C2 comprises the CDRs of antibody C2 in Table 20. In some cases, antibody C2 comprises a heavy chain framework comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody C2 comprises a light chain framework comprising SEQ ID NO: 303 (EIVLTQSPGTLSLSPGERATLSC[LCDR1]WYQQKPGQAPRX10X11IY[LCDR2]GIPDRES GSGSGTDFTLTISRLEPEDFAVYYC[LCDR3]FGGGTKLEIK), wherein X10=L or P and X11=L or W. In some cases, antibody C2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C2 comprises a constant region comprising reduced ADCC and/or CDC as compared to IgG1. For instance, antibody C2 comprises a human IgG1 Fc region comprising (a) 297A, 297Q, 297G, or 297D, (b) 279F, 279K, or 279L, (c) 228P, (d) 235A, 23SE, 235G, 235Q, 235R, or 235S, (e) 237A, 237E, 237K, 237N, or 237R, (1) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N. (z) 292E, 292F, 292G, or 2921, (aa) 293S, (bb) 301 W. (cc) 304E, (dd) 311E, 311G, or 311S, (ee) 316F, (ff) 328V, (gg) 330R, (hh) 339E or 339L, (ii) 343I or 343V, (j) 373A, 373G, or 3738, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (eee) G237A, (fff) F241A, (ggg) V264A, (hhh) D265A, (iii) D265A and N297A, (j) D265A and N297G, (kkk) D270A, (III) A330L, (mmm) P331A or P331S, or (nnn) any combination of (a)-(uu), per Kabat numbering. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody C2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody C2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody C2 is expressed from FreeStyle 293-F cells at an expression level of between about 2 μg/mL to about 60 g/mL. In some cases, antibody C2 is expressed from FreeStyle 293-F cells at an expression level of between about 10 μg/mL to about 60 μg/mL.

In some embodiments, an anti-TL1A antibody comprises antibody D2. As used herein, antibody D2 comprises the CDRs of antibody D2 in Table 20. In some cases, antibody D2 comprises a heavy chain framework comprising SEQ ID NO: 302 (XIVQLVQSGAEVKKPGASVKVSCKAS[HCDRIJWVX2QX3PGQGLEWX4G|HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or 1, X5=V or A, X6=M or 1, X7=R or T, X8=V or A, and X9=M or L. In some cases, antibody D2 comprises a light chain framework comprising SEQ ID NO: 303 (EIVLTQSPGTLSLSPGERATLSC[LCDR1]WYQQKPGQAPRX10X11TY[LCDR2]GIPDRFS GSGSGTDFTLTISRLEPEDFAVYYC[LCDR3]FGGGTKLEIK), wherein X10=L or P and X11=L or W. In some cases, antibody D2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D2 comprises a constant region comprising reduced ADCC and/or CDC as compared to IgG1. For instance, antibody D2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (1) 329A, 329G, 329Y, or 329R (k) 331S, (1) 236F or 236R, (m) 238A, 238B, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q. (w) 270A, 270G, 270M, or 270N, (x) 271T, (y) 272N, (z) 292B, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (ij) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P. (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R. (eee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody D2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody D2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 g/mL as determined by the method described in Example 2. In some cases, antibody D2 is expressed from FreeStyle 293-F cells at an expression level of between about 2 μg/mL to about 60 μg/mL. In some cases, antibody D2 is expressed from FreeStyle 293-F cells at an expression level of between about 10 μg/mL to about 60 μg/mL.

In some embodiments, an anti-TL1A antibody comprises antibody E2. As used herein, antibody E2 comprises the CDRs of antibody E2 in Table 20. In some cases, antibody E2 comprises a heavy chain framework comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I. X5=V or A, X6=M or I, X7=R or T, X8=V or A, and X9=M or L. In some cases, antibody E2 comprises a light chain framework comprising SEQ ID NO: 303 (EIVLTQSPGTLSLSPGERATLSC[LCDR1]WYQQKPGQAPRX10X11IY[LCDR2]GIPDRES GSGSGTDFTLTISRLEPEDFAVYYC[LCDR3]FGGGTKLEIK), wherein X10=L or P and X11=L or W. In some cases, antibody E2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E2 comprises a constant region comprising reduced ADCC and/or CDC as compared to IgG1. For instance, antibody E2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V. (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q. (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G. (rr) 439B, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (eee) G237A. (fff) F241A, (ggg) V264A. (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody E2 comprises a constant region comprising at least about 85%, 87%, 88%, 89%, 90%, 91%, 92%, 93%, 94%, 95%, 96%, 97%, 98% 99%, or 100% identity to SEQ ID NO: 320. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 346, In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody E2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody E2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5. 57, 58, 59 or 60 μg/mL as determined by the method described in Example 2. In some cases, antibody E2 is expressed from FreeStyle 293-F cells at an expression level of between about 2 μg/mL to about 60 μg/mL. In some cases, antibody E2 is expressed from FreeStyle 293-F cells at an expression level of between about 10 g/mL to about 60 μg/mL.

In some embodiments, an anti-TL1A antibody comprises antibody F2. As used herein, antibody F2 comprises the CDRs of antibody F2 in Table 20. In some cases, antibody F2 comprises a heavy chain framework comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K. X3=A or R, X4=M or I. X5=V or A, X6=M or I, X7=R or T. X8=V or A, and X9=M or L. In some cases, antibody F2 comprises a light chain framework comprising SEQ ID NO: 303 (EIVLTQSPGTLSLSPGERATLSC[LCDR1]WYQQKPGQAPRX10X11IY[LCDR2]GIPDRES GSGSGTDFTLTISRLEPEDFAVYYC[LCDR3]FGGGTKLEIK), wherein X10=L or P and X11=L or W. In some cases, antibody F2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F2 comprises a constant region comprising reduced ADCC and/or CDC as compared to IgG1. For instance, antibody F2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (1) 327Q or 327T, (1) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 2658, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V. (ij) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A. (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (cee) G237A, (fff) F241A, (ggg) V264A. (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody F2 comprises a constant region comprising at least about 85%, 87%, 88%, 89%, 90%, 91%, 92%, 93%, 94%, 95% 96%, 97%, 98%, 99%, or 100% identity to SEQ ID NO: 320. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody F2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody F2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5. 57, 58, 59 or 60 μg/mL as determined by the method described in Example 2. In some cases, antibody F2 is expressed from FreeStyle 293-F cells at an expression level of between about 2 μg/mL to about 60 μg/mL. In some cases, antibody F2 is expressed from FreeStyle 293-F cells at an expression level of between about 10 μg/mL to about 60 μg/mL.

In some embodiments, an anti-TL1A antibody comprises antibody G2. As used herein, antibody G2 comprises the CDRs of antibody G2 in Table 20. In some cases, antibody G2 comprises a heavy chain framework comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K. X3=A or R, X4=M or I, X5=V or A, X6=M or I, X7=R or T, X8=V or A, and X9=M or L. In some cases, antibody G2 comprises a light chain framework comprising SEQ ID NO: 303 (EIVLTQSPGTLSLSPGERATLSC[LCDR1]WYQQKPGQAPRX10X11IY[LCDR2]GIPDRES GSGSGTDFTLTISRLEPEDFAVYYC[LCDR3]FGGGTKLEIK), wherein X10=L or P and X11=L or W. In some cases, antibody G2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G2 comprises a constant region comprising reduced ADCC and/or CDC as compared to IgG1. For instance, antibody G2 comprises a human IgG1 Fe region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A. (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 27IT, (y) 272N, (z) 292E, 292F, 292G, or 292I, (aa) 293S, (bb) 301 W, (cc) 304E, (dd) 311E, 311G, or 311S, (ec) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P. (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A. (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (eee) G237A, (fff) F241A, (ggg) V264A, (hhh) D265A, (iji) D265A and N297A, (jjj) D265A and N297G, (kkk) D270A. (III) A330L, (mmm) P331A or P331S, or (nnn) any combination of (a)-(uu), per Kabat numbering. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody G2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody G2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody G2 is expressed from FreeStyle 293-F cells at an expression level of between about 2 μg/mL to about 60 g/mL. In some cases, antibody G2 is expressed from FreeStyle 293-F cells at an expression level of between about 10 μg/mL to about 60 μg/mL.

In some embodiments, an anti-TL1A antibody comprises antibody H2. As used herein, antibody H2 comprises the CDRs of antibody H2 in Table 20. In some cases, antibody H2 comprises a heavy chain framework comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R. X4=M or I, X5=V or A, X6=M or I, X7=R or T, X8=V or A, and X9=M or L. In some cases, antibody H2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody H2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H2 comprises a constant region comprising reduced ADCC and/or CDC as compared to IgG1. For instance, antibody H2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (ij) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (77) L234A, L235E, and G237A, (aaa), L234A, L235E, G237A, and P331S (bbb) L234A, L235A, G237A, P238S, H268A, A330S, and P331S (IgGlo, (ccc) L234A, L235A, and P329A, (ddd) G236R and L328R, (cee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody H2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody H2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody H2 is expressed from FreeStyle 293-F cells at an expression level of between about 2 μg/mL to about 60 μg/mL. In some cases, antibody H2 is expressed from FreeStyle 293-F cells at an expression level of between about 10 μg/mL to about 60 μg/mL.

The TL1A antibodies described herein bind to specific regions or epitopes of human TL1A demonstrated herein as useful to inhibit interferon gamma secretion from T lymphocytes. Various embodiments provide for an anti-TL1A antibody that binds to the same region of a TL1A protein or portion thereof as a reference antibody such as the anti-TL1A antibodies described herein. In some embodiments, the reference antibody comprises antibody A, B, C, D, E, F, G, H, A2, B2, C2, D2, E2, F2, G2, or H2, or a combination thereof. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 104, and a light chain comprising a sequence at least about 90%, 92%, 93%, 94%, 95%, 96%, 97%, 98%, 99% or 100% identical to SEQ ID NO: 201. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 107, and a light chain comprising a sequence at least about 90%, 92%, 93%, 94%, 95%, 96%, 97%, 98%, 99% or 100% identical to SEQ ID NO: 201.

Non-limiting methods for determining whether an anti-TL1A antibody (i.e. test antibody) binds to the same region of a TL1A protein or portion thereof as an antibody described herein are provided. An exemplary embodiment comprises a competition assay. For instance, the method comprises determining whether the test antibody can compete with binding between the reference antibody and the TL1A protein or portion thereof, or determining whether the reference antibody can compete with binding between the test antibody and the TL1A protein or portion thereof. Exemplary methods include use of surface plasmon resonance to evaluate whether an anti-TL1A antibody can compete with the binding between TL1A and another anti-TL1A antibody. In some cases, surface plasmon resonance is utilized in the competition assay. Non-limiting methods are described in the examples.

In certain embodiments, disclosed herein are antibodies that compete for binding TL1A with the antibodies described herein. In certain embodiments, disclosed herein are antibodies that bind a discrete epitope that overlaps with an epitope of TL1A bound by an antibody described herein. In certain embodiments, disclosed herein are antibodies that bind the same epitope of TL1A, overlap with an epitope of TL1A by one or more amino acid residues, or that compete for binding to an epitope of TL1A with an antibody or fragment thereof that comprises a heavy chain variable region comprising the amino acid sequence of SEQ ID NO: 104; and a light chain variable region comprising the amino acid of SEQ ID NO: 201. In certain embodiments, disclosed herein are antibodies that bind the same epitope of TL1A, overlap with an epitope of TL1A by one or more amino acid residues, or that compete for binding to an epitope of TL1A with an antibody or fragment thereof that comprises a heavy chain variable region comprising the amino acid sequence of SEQ ID NO: 107; and a light chain variable region comprising the amino acid of SEQ ID NO: 201.

### 5.3.1.5 Additional Antibody Embodiments

In one aspect, provided herein, is an antibody or antigen binding fragment thereof that binds to TL1A, comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework. In some embodiments, the amino acid modification comprises: (a) a modification at amino acid position 47 in the heavy chain variable region; (b) a modification at amino acid position 45 in the heavy chain variable region; (c) a modification at amino acid position 55 in the heavy chain variable region; (d) a modification at amino acid position 78 in the heavy chain variable region; (e) a modification at amino acid position 80 in the heavy chain variable region; (f) a modification at amino acid position 82 in the heavy chain variable region; (g) a modification at amino acid position 89 in the heavy chain variable region; or (h) a modification at amino acid position 91 in the heavy chain variable region; per Aho or Kabat numbering; or a combination of two or more modifications selected from (a) to (h). In some embodiments, (a) the amino acid modification is at position 47 in the heavy chain variable region, and the amino acid at position 47 is R, N, D, C, Q, E, G, H, I, L, K, M, F, P, S, T, W, Y, or V; (b) the amino acid modification is at position 45 in the heavy chain variable region, and the amino acid at position 45 is A, N, D, C, Q, E, G, H, I, L. K, M, F, P. S. T, W, Y, or V: (c) the amino acid modification is at position 55 in the heavy chain variable region, and the amino acid at position 55 is A, R, N, D, C, Q, E, G, H, I, L, K, F, P, S, T, W, Y, or V; (d) the amino acid modification is at position 78 in the heavy chain variable region, and the amino acid at position 78 is A, R, N. D, C, Q, E, G, H, I, L, K, M, F, P. S, T, W, or Y; (e) the amino acid modification is at position 80 in the heavy chain variable region, and the amino acid at position 80 is A. R. N. D, C, Q, E, G, H, I, L, K, F, P, S, T, W, Y, or V; (f) the amino acid modification is at position 82 in the heavy chain variable region, and the amino acid at position 82 is A, N, D, C, Q, E, G, H, I, L, K, M, F, P, S, T, W, Y, or V; (g) the amino acid modification is at position 89 in the heavy chain variable region, and the amino acid at position 89 is A, R, N, D, C, Q, E, G, H, I, L, K, M, F, P. S. T, W, or Y; or (h) the amino acid modification is at position 91 in the heavy chain variable region, and the amino acid at position 91 is A. R. N. D. C. Q. B, G, H, I, L, K, F, P. S. T, W, Y, or V; or a combination of two or more modifications selected from (a) to (h). In some embodiments, the amino acid modifications comprise one or more of: A47R, R45K, M55I, V78A, M801, R82T. V89A, M91L in the heavy chain variable region, per Abo or Kabat numbering. In some embodiments, the amino acid modification comprises: (a) a modification at amino acid position 54 in the light chain variable region; and/or (b) a modification at amino acid position 55 in the light chain variable region; per Aho or Kabat numbering. In some embodiments. (a) the amino acid modification is at position 54 of the light chain variable region, and the amino acid at position 54 is A, R, N, D, C, Q, E, G, H, I, K, M, F, P, S, T, W, Y, or V; and/or (b) the amino acid modification is at position 55 of the light chain variable region, and the amino acid at position 55 is A, R, N, D, C, Q, E, G, H, I, K, M, F, P, S, T, W, Y, or V. In some embodiments, the amino acid modifications comprise L54P and/or L55 W in the light chain variable region, per Aho or Kabat numbering. In some embodiments, the antibody or antigen binding fragment comprises a heavy chain CDR1 as set forth by SEQ ID NO: 1, a heavy chain CDR2 as set forth by any one of SEQ ID NOS: 2-5, a heavy chain CDR3 as set forth by any one of SEQ ID NOS: 6-9, a light chain CDR1 as set forth by SEQ ID NO: 10, a light chain CDR2 as set forth by SEQ ID NO: 11, and a light chain CDR3 as set forth by any one of SEQ ID NOS: 12-15. In some embodiments, the antibody or antigen binding fragment comprises a heavy chain CDR1 as set forth by SEQ ID NO: 1, a heavy chain CDR2 as set forth by SEQ ID NO: 2, a heavy chain CDR3 as set forth by SEQ ID NO: 6, a light chain CDR1 as set forth by SEQ ID NO: 10, a light chain CDR2 as set forth by SEQ ID NO: 11, and a light chain CDR3 as set forth by SEQ ID NO: 12. In some embodiments, the antibody or antigen binding fragment comprises comprising a heavy chain framework (FR) I as set forth by SEQ ID NO: 304, a heavy chain FR2 as set forth by SEQ ID NO: 305 or SEQ ID NO: 313, a heavy chain FR3 as set forth by any one of SEQ ID NOS: 306, 307, 314, or 315, a heavy chain FR4 as set forth by SEQ ID NO: 308, a light chain FR1 as set forth by SEQ ID NO: 309, a light chain FR2 as set forth by SEQ ID NO: 310, a light chain FR3 as set forth by SEQ ID NO: 311, or a light chain FR4 as set forth by SEQ ID NO: 312, or a combination thereof. In some embodiments, the antibody or antigen binding fragment comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V. (jj) 373A, 373G, or 3738, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V. (tt) E233P, (uu) L235E, (vv) L234A and L235A, (ww) L234A, L235A, and G237A. (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (jj) D265A and N297G, (kkk) D270A, (III) A330L, (mmm) P331A or P331S, or (nnn) any combination of two or more selected from (a)-(uu), per Kabat numbering. In some embodiments, the antibody or antigen binding fragment comprises a human IgG4 Fc region. In some embodiments, the antibody or antigen binding fragment comprises a Fc region comprising a sequence at least about 90%, 91%, 92%, 93%, 94%, 95%, 96%, 97%, 98%, 99%, or 100% identical to any one of SEQ ID NOS: 320-362. In some embodiments, the antibody or antigen binding fragment comprises at least about 80% monomeric fraction as determined by size exclusion chromatography. In some embodiments, the antibody or antigen binding fragment expresses at least about 20 μg/ml total antibody, optionally about 20 μg/ml and 70 μg/mL total antibody.

In another aspect, provided herein is an antibody or antigen binding fragment thereof that binds to tumor necrosis factor-like protein 1A (TL1A), comprising a heavy chain variable domain comprising an amino acid sequence at least 96% identical to SEQ ID NO: 104, and a light chain variable domain comprising an amino acid sequence at least 97% identical to SEQ ID NO: 201. In some embodiments, the heavy chain variable domain comprises an amino acid sequence at least 97% identical to SEQ ID NO: 104. In some embodiments, the heavy chain variable domain comprises an amino acid sequence at least 98% identical to SEQ ID NO: 104. In some embodiments, the heavy chain variable domain comprises an amino acid sequence at least 99% identical to SEQ ID NO: 104. In some embodiments, the heavy chain variable domain comprises SEQ ID NO: 104. In some embodiments, the light chain variable domain comprises an amino acid sequence at least 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

In another aspect, provided herein is an antibody or antigen binding fragment thereof that binds to tumor necrosis factor-like protein 1A (TL1A), comprising a heavy chain variable domain comprising an amino acid sequence at least about 99% identical to any one of SEQ ID NOS: 101-135, and a light chain variable domain comprising an amino acid sequence at least about 99% identical to any one of SEQ ID NOS: 201-206.

In some embodiments, antibodies or antigen binding fragments described herein comprise a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V. (r) 264S, (s) 265H, 265K, 265S, 265Y, or 265A, (t) 267G, 267H, 267I, or 267K, (u) 268K. (v) 269N or 269Q, (w) 270A, 270G, 270M, or 270N, (x) 271T, (y) 272N, (z) 292E, 292F, 292G, or 292I, (aa) 293S, (bb) 301 W, (cc) 304E, (dd) 311E, 311G, or 31IS, (ee) 316F, (ff) 328V, (gg) 330R, (hh) 339E or 339L, (ii) 3431 or 343V, (jj) 373A, 373G, or 3738, (kk) 376E, 376 W, or 376Y, (11) 380D. (mm) 382D or 382P, (nn) 385P, (co) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L23SE, and P331S, (zz) L234A, L235E, and G237A. (aaa), L234A, L235E, G237A, and P331S (bbb) L234A. L235A, G237A, P238S. H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (jj) D265A and N297G, (kkk) D270A, (III) A330L, (mmm) P331A or P331S, or (nnn) any combination of two or more selected from (a)-(uu), per Kabat numbering. In some embodiments, antibodies or antigen binding fragments described herein comprise a human IgG4 Fc region. In some embodiments, antibodies or antigen binding fragments described herein comprise a Fc region comprising a sequence at least about 90%, 91%, 92%, 93%, 94%, 95%, 96%, 97%, 98%, 99%, or 100% identical to any one of SEQ ID NOS: 320-362. In some embodiments, antibodies or antigen binding fragments described herein comprise at least about 80% monomeric fraction as determined by size exclusion chromatography. In some embodiments, antibodies or antigen binding fragments described herein express at least about 20 μg/ml total antibody, optionally about 20 μg/ml and 70 μg/mL total antibody.

In another aspect, provided herein is an antibody or antigen binding fragment thereof that binds to TL1A, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; (f) an LCDR3 comprising an amino acid sequence set forth by any one of SEQ ID NOS: 12-15; and a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1. In some embodiments, the human IgG1 comprises SEQ ID NO: 320. In some embodiments, the ADCC function of the Fc region comprising reduced ADCC is at least about 50% reduced as compared to human IgG1. In some embodiments, the CDC function of the Fc region comprising reduced CDC is at least about 50% reduced as compared to human IgG1. In some embodiments, the Fc region comprises a human IgG1 comprising (a) 297A, 297Q, 297G, or 297D. (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (il) 3431 or 343V. (ij) 373A, 373G, or 373S. (kk) 376E, 376 W, or 376Y, (11) 380D, (mm) 382D or 382P, (nn) 385P, (co) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L23SE, G237A, and P331S (bbb) L234A, L23SA, G237A, P238S, H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some embodiments, the antibody or antigen binding fragment comprises a (i) human IgG4 Fc region or (ii) a human IgG4 Fc region comprising (a) S228P. (b) S228P and L235E, or (c) S228P, F234A, and L235A, per Kabat numbering. In some embodiments, the antibody or antigen binding fragment comprises a human IgG2 Fc region; IgG2-IgG4 cross-subclass Fc region: IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). In some embodiments, the Fc region comprises a human IgG1 with a substitution selected from 329A, 329G, 329Y, 331S, 236F, 236R, 238A, 238E, 238G, 238H, 238I, 238V, 238 W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering. In some embodiments, the antibody or antigen binding fragment comprises a sequence at least about 90%, 91%, 92%, 93%, 94%, 95%, 96%, 97%, 98%, 99%, or 100% identical to any one of SEQ ID NOS: 320-362. In some embodiments, the HCDR1 comprises SEQ ID NO: 1, HCDR2 comprises any one of SEQ ID NOS: 2-5, HCDR3 comprises any one of SEQ ID NOS: 6-9, LCDR1 comprises SEQ ID NO: 10, LCDR2 comprises SEQ ID NO: 11, and LCDR3 comprises any one of SEQ ID NOS: 12-15. In some embodiments, the Fc region comprises any one of SEQ ID NOs: 401-413 or a sequence at least about 90%, 91%, 92%, 93%, 94%, 95%, 96%, 97%, 98%, or 99% identical to any one of SEQ ID NOs: 401-413. In some embodiments, the heavy chain comprises any one of SEQ ID NOs: 501-513 or a sequence at least about 90%, 91%, 92%, 93%, 94%, 95%, 96%, 97%, 98%, or 99% identical to any one of SEQ ID NOs: 501-513. In some embodiments, the light chain comprises any one of SEQ ID NO: 514 or a sequence at least about 90%, 91%, 92%, 93%, 94%, 95%, 96%, 97%, 98%, or 99% identical to any one of SEQ ID NO: 514. In some embodiments, the HCDR2 comprises SEQ ID NO: 2, the HCDR3 comprises SEQ ID NO: 6, and the LCDR3 comprises SEQ ID NO: 12, and wherein the Fc region comprises any one of SEQ ID NOs: 401-413 or a sequence at least about 90%, 91%, 92%, 93%, 94%, 95%, 96%, 97%, 98%, or 99% identical to any one of SEQ ID NOs: 401-413. In some embodiments, the antibody or antigen binding fragment comprises at least about 80% monomeric fraction as determined by size exclusion chromatography. In some embodiments, the antibody or antigen binding fragment expresses at least about 20 μg/ml total antibody, optionally about 20 μg/ml and 70 μg/mL total antibody.

Further provided are methods of treating fibrosis or an intestinal inflammatory condition, disease, or disorder in a subject in need thereof, the method comprising administering to the subject an antibody or antigen binding fragment of any antibody or antigen binding fragment disclosed herein. In some embodiments, the subject has fibrosis. In some embodiments, the subject has an intestinal inflammatory condition, disease, or disorder. In some embodiments, the intestinal inflammatory condition, disease, or disorder comprises inflammatory bowel disease (IBD). In some embodiments, the IBD comprises Crohn's Disease. In some embodiments, the IBD comprises ulcerative colitis.

In some embodiments, antibodies or antigen binding fragments described herein comprise are present in a liquid composition a concentration of the antibody or antigen binding fragment of 10 mg/ml to 170 mg/ml. In some embodiments, the antibody or antigen binding fragment thereof is at a concentration of 10 mg/ml to 170 mg/ml. In some embodiments, the viscosity is from about 4 to about 30 mPa-s (millipascal-second (mPa·s)). In some embodiments, the viscosity is from about 4 to about 10 mPa-s (millipascal-second (mPa·s)).

In another aspect, provided herein, is an antibody or antigen binding fragment thereof that binds to TL1A, comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework. In some embodiments, the heavy chain variable framework region and the light chain variable framework region collectively comprise 13, 12, 11, 10, 9, 8, 7, 6, 5, 4, 3, 2, 1, or no amino acid modifications from the human IGHV1-46*02 framework and the human IGKV3-20 framework. In some embodiments, the amino acid modification comprises a modification at amino acid position 1 in the heavy chain variable region, per Aho or Kabat numbering. In some embodiments, the amino acid at position I comprises A, R, N, D, C, E, G, H, I, L, K, M, F, P, S, T, W, Y, or V. In some embodiments, the amino acid at position 1 comprises a hydrophobic amino acid, a hydrophilic amino acid, or an amphipathic amino acid. In some embodiments, the amino acid at position I comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 1 comprises E. In some embodiments, the amino acid modification comprises a modification at amino acid position 45 in the heavy chain variable region, per Aho or Kabat numbering. In some embodiments, the amino acid at position 45 comprises A, N, D, C, Q, E, G, H, I, L, K, M, F, P, S, T, W, Y, or V. In some embodiments, the amino acid at position 45 comprises a hydrophobic amino acid, a hydrophilic amino acid, or an amphipathic amino acid. In some embodiments, the amino acid at position 45 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 45 comprises K. In some embodiments, the amino acid modification comprises a modification at amino acid position 47 in the heavy chain variable region, per Aho or Kabat numbering. In some embodiments, the amino acid at position 47 comprises R, N, D, C, Q, E, G, H, I, L, K, M, F, P, S, T, W, Y, or V. In some embodiments, the amino acid at position 47 comprises a hydrophobic amino acid, a hydrophilic amino acid, or an amphipathic amino acid. In some embodiments, the amino acid at position 47 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 47 comprises R. In some embodiments, the amino acid modification comprises a modification at amino acid position 55 in the heavy chain variable region, per Aho or Kabat numbering. In some embodiments, the amino acid at position 55 comprises A, R, N, D, C, Q, E, G, H, I, L, K, F, P, S, T, W, Y, or V. In some embodiments, the amino acid at position 55 comprises a hydrophobic amino acid, a hydrophilic amino acid, or an amphipathic amino acid. In some embodiments, the amino acid at position 55 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 55 comprises I. In some embodiments, the amino acid modification comprises a modification at amino acid position 78 in the heavy chain variable region, per Aho or Kabat numbering. In some embodiments, the amino acid at position 78 comprises A, R, N, D, C, Q, E, G, H, I, L, K, M. F, P, S, T, W, or Y. In some embodiments, the amino acid at position 78 comprises a hydrophobic amino acid, a hydrophilic amino acid, or an amphipathic amino acid. In some embodiments, the amino acid at position 78 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 78 comprises A. In some embodiments, the amino acid modification comprises a modification at amino acid position 80 in the heavy chain variable region, per Aho or Kabat numbering. In some embodiments, the amino acid at position 80 comprises A, R, N. D. C. Q. E, G, H, I, L, K, F, P. S. T, W. Y, or V. In some embodiments, the amino acid at position 80 comprises a hydrophobic amino acid, a hydrophilic amino acid, or an amphipathic amino acid. In some embodiments, the amino acid at position 80 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 80 comprises I. In some embodiments, the amino acid modification comprises a modification at amino acid position 82 in the heavy chain variable region, per Aho or Kabat numbering. In some embodiments, the amino acid at position 82 comprises A, N, D, C, Q, E, G, H, I, L, K, M, F, P, S, T, W, Y, or V. In some embodiments, the amino acid at position 82 comprises a hydrophobic amino acid, a hydrophilic amino acid, or an amphipathic amino acid. In some embodiments, the amino acid at position 82 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 82 comprises T. In some embodiments, the amino acid modification comprises a modification at amino acid position 89 in the heavy chain variable region, per Aho or Kabat numbering. In some embodiments, the amino acid at position 89 comprises A, R, N, D, C, Q, E, G, H, I, L, K, M, F, P, S, T, W, or Y. In some embodiments, the amino acid at position 89 comprises a hydrophobic amino acid, a hydrophilic amino acid, or an amphipathic amino acid. In some embodiments, the amino acid at position 89 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 89 comprises A. In some embodiments, the amino acid modification comprises a modification at amino acid position 91 in the heavy chain variable region, per Aho or Kabat numbering. In some embodiments, the amino acid at position 91 comprises A, R, N, D, C, Q, E, G, H, I, L, K, F, P, S, T, W, Y, or V. In some embodiments, the amino acid at position 91 comprises a hydrophobic amino acid, a hydrophilic amino acid, or an amphipathic amino acid. In some embodiments, the amino acid at position 91 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 91 comprises L.

In some embodiments, the amino acid modifications comprise one or more of: Q1E, R45K, A47R, M55I, V78A, M801, R82T, V89A, M91L in the heavy chain variable region, per Aho or Kabat numbering. In some embodiments, the amino acid modifications comprise Q1E. In some embodiments, the amino acid modifications comprise R45K. In some embodiments, the amino acid modifications comprise A47R. In some embodiments, the amino acid modifications comprise M551. In some embodiments, the amino acid modifications comprise V78A. In some embodiments, the amino acid modifications comprise M80I. In some embodiments, the amino acid modifications comprise R82T. In some embodiments, the amino acid modifications comprise V89A. In some embodiments, the amino acid modifications comprise M91L.

In some embodiments, the amino acid modification comprises a modification at amino acid position 54 in the light chain variable region, per Aho or Kabat numbering. In some embodiments, the amino acid at position 54 comprises A, R, N, D, C, Q, E, G, H, I, K, M, F, P, S, T, W, Y, or V. In some embodiments, the amino acid at position 54 comprises a hydrophobic amino acid, a hydrophilic amino acid, or an amphipathic amino acid. In some embodiments, the amino acid at position 54 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 54 comprises P. In some embodiments, the amino acid modification comprises a modification at amino acid position 55 in the light chain variable region, per Aho or Kabat numbering. In some embodiments, the amino acid at position 55 comprises A, R, N, D, C, Q, E, G, H, I, K, M, F, P, S, T, W, Y, or V. In some embodiments, the amino acid at position 55 comprises a hydrophobic amino acid, a hydrophilic amino acid, or an amphipathic amino acid. In some embodiments, the amino acid at position 55 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 55 comprises W.

In some embodiments, the amino acid modifications comprise L54P and/or L55 W in the light chain variable region, per Aho or Kabat numbering. In some embodiments, the amino acid modifications comprise L54P. In some embodiments, the amino acid modifications comprise L55W.

In some embodiments, the antibody or antigen binding fragment comprises a heavy chain CDR1 as set forth by SEQ ID NO: 1. In some embodiments, the antibody or antigen binding fragment comprises a heavy chain CDR2 as set forth by SEQ ID NO: 2. In some embodiments, the antibody or antigen binding fragment comprises a heavy chain CDR2 as set forth by SEQ ID NO: 3. In some embodiments, the antibody or antigen binding fragment comprises a heavy chain CDR2 as set forth by SEQ ID NO: 4. In some embodiments, the antibody or antigen binding fragment comprises a heavy chain CDR2 as set forth by SEQ ID NO: 5. In some embodiments, the antibody or antigen binding fragment comprises a heavy chain CDR3 as set forth by SEQ ID NO: 6. In some embodiments, the antibody or antigen binding fragment comprises a heavy chain CDR3 as set forth by SEQ ID NO: 7. In some embodiments, the antibody or antigen binding fragment comprises a heavy chain CDR3 as set forth by SEQ ID NO: 8. In some embodiments, the antibody or antigen binding fragment comprises a heavy chain CDR3 as set forth by SEQ ID NO: 9. In some embodiments, the antibody or antigen binding fragment comprises a light chain CDR1 as set forth by SEQ ID NO: 10. In some embodiments, the antibody or antigen binding fragment comprises a light chain CDR2 as set forth by SEQ ID NO: 11. In some embodiments, the antibody or antigen binding fragment comprises a light chain CDR3 as set forth by SEQ ID NO: 12. In some embodiments, the antibody or antigen binding fragment comprises a light chain CDR3 as set forth by SEQ ID NO: 13. In some embodiments, the antibody or antigen binding fragment comprises a light chain CDR3 as set forth by SEQ ID NO: 14. In some embodiments, the antibody or antigen binding fragment comprises a light chain CDR3 as set forth by SEQ ID NO: 15.

In some embodiments, the antibody or antigen binding fragment comprises a heavy chain FR1 as set forth by SEQ ID NO: 304. In some embodiments, the antibody or antigen binding fragment comprises a heavy chain FR2 as set forth by SEQ ID NO: 305. In some embodiments, the antibody or antigen binding fragment comprises a heavy chain FR2 as set forth by SEQ ID NO: 313. In some embodiments, the antibody or antigen binding fragment comprises a heavy chain FR3 as set forth by SEQ ID NO: 306. In some embodiments, the antibody or antigen binding fragment comprises a heavy chain FR3 as set forth by SEQ ID NO: 307. In some embodiments, the antibody or antigen binding fragment comprises a heavy chain FR3 as set forth by SEQ ID NO: 314. In some embodiments, the antibody or antigen binding fragment comprises a heavy chain FR3 as set forth by SEQ ID NO: 315. In some embodiments, the antibody or antigen binding fragment comprises a heavy chain FR4 as set forth by SEQ ID NO: 308. In some embodiments, the antibody or antigen binding fragment comprises a light chain FR1 as set forth by SEQ ID NO: 309. In some embodiments, the antibody or antigen binding fragment comprises a light chain FR2 as set forth by SEQ ID NO: 310. In some embodiments, the antibody or antigen binding fragment comprises a light chain FR3 as set forth by SEQ ID NO: 311. In some embodiments, the antibody or antigen binding fragment comprises a light chain FR4 as set forth by SEQ ID NO: 312.

In another aspect, provided herein is an antibody or antigen binding fragment thereof that binds to TL1A, comprising: (a) a heavy chain variable region comprising SEQ ID NO: 301 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), and (b) a light chain variable region comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC|LCDR3|FGGGTKLEIK), wherein each of X1-X11 is independently selected from A, R. N, D, C, Q, E, G, H, I, L, K, M, F, P. S, T, W, Y, or V.

In some embodiments, X1 comprises a hydrophobic amino acid, a hydrophilic amino acid, or an amphipathic amino acid. In some embodiments, X1 comprises an aliphatic amino acid, an aromatic amino acid, an acidic amino acid, a basic amino acid, a hydroxylic amino acid, a sulfur-containing amino acid, or an amidic amino acid. In some embodiments, X2 comprises a hydrophobic amino acid, a hydrophilic amino acid, or an amphipathic amino acid. In some embodiments, X2 comprises an aliphatic amino acid, an aromatic amino acid, an acidic amino acid, a basic amino acid, a hydroxylic amino acid, a sulfur-containing amino acid, or an amidic amino acid. In some embodiments, X3 comprises a hydrophobic amino acid, a hydrophilic amino acid, or an amphipathic amino acid. In some embodiments. X3 comprises an aliphatic amino acid, an aromatic amino acid, an acidic amino acid, a basic amino acid, a hydroxylic amino acid, a sulfur-containing amino acid, or an amidic amino acid. In some embodiments, X4 comprises a hydrophobic amino acid, a hydrophilic amino acid, or an amphipathic amino acid. In some embodiments, X4 comprises an aliphatic amino acid, an aromatic amino acid, an acidic amino acid, a basic amino acid, a hydroxylic amino acid, a sulfur-containing amino acid, or an amidic amino acid. In some embodiments, X5 comprises a hydrophobic amino acid, a hydrophilic amino acid, or an amphipathic amino acid. In some embodiments, X5 comprises an aliphatic amino acid, an aromatic amino acid, an acidic amino acid, a basic amino acid, a hydroxylic amino acid, a sulfur-containing amino acid, or an amidic amino acid. In some embodiments, X6 comprises a hydrophobic amino acid, a hydrophilic amino acid, or an amphipathic amino acid. In some embodiments, X6 comprises an aliphatic amino acid, an aromatic amino acid, an acidic amino acid, a basic amino acid, a hydroxylic amino acid, a sulfur-containing amino acid, or an amidic amino acid. In some embodiments, X7 comprises a hydrophobic amino acid, a hydrophilic amino acid, or an amphipathic amino acid. In some embodiments, X7 comprises an aliphatic amino acid, an aromatic amino acid, an acidic amino acid, a basic amino acid, a hydroxylic amino acid, a sulfur-containing amino acid, or an amidic amino acid. In some embodiments, X8 comprises a hydrophobic amino acid, a hydrophilic amino acid, or an amphipathic amino acid. In some embodiments, X8 comprises an aliphatic amino acid, an aromatic amino acid, an acidic amino acid, a basic amino acid, a hydroxylic amino acid, a sulfur-containing amino acid, or an amidic amino acid. In some embodiments. X9 comprises a hydrophobic amino acid, a hydrophilic amino acid, or an amphipathic amino acid. In some embodiments, X9 comprises an aliphatic amino acid, an aromatic amino acid, an acidic amino acid, a basic amino acid, a hydroxylic amino acid, a sulfur-containing amino acid, or an amidic amino acid. In some embodiments, X10 comprises a hydrophobic amino acid, a hydrophilic amino acid, or an amphipathic amino acid. In some embodiments, X10 comprises an aliphatic amino acid, an aromatic amino acid, an acidic amino acid, a basic amino acid, a hydroxylic amino acid, a sulfur-containing amino acid, or an amidic amino acid. In some embodiments. X11 comprises a hydrophobic amino acid, a hydrophilic amino acid, or an amphipathic amino acid. In some embodiments, X11 comprises an aliphatic amino acid, an aromatic amino acid, an acidic amino acid, a basic amino acid, a hydroxylic amino acid, a sulfur-containing amino acid, or an amidic amino acid.

In some embodiments, X1=Q or E, X2=R or K, X3=A or R, X4=M or I, X5=V or A, X6=M or I, X7=R or T. X8=V or A, X9=M or L, X10=L or P, and X11=L or W. In some embodiments, X1=Q. In some embodiments, X1=E. In some embodiments, X2=R. In some embodiments, X2=K. In some embodiments, X3=A. In some embodiments, X3=R. In some embodiments, X4=M. In some embodiments, X4=1. In some embodiments, X5=V. In some embodiments, X5=A. In some embodiments, X6=M. In some embodiments, X6=I. In some embodiments, X7=R. In some embodiments, X7=T. In some embodiments, X8=V. In some embodiments, X8=A. In some embodiments, X9=M. In some embodiments, X9=L. In some embodiments, X10=L. In some embodiments, X10=P. In some embodiments, X11=L. In some embodiments, X11=W.

In some embodiments, HCDR1 comprises SEQ ID NO: 1. In some embodiments, HCDR2 comprises SEQ ID NO: 2. In some embodiments, HCDR2 comprises SEQ ID NO: 3. In some embodiments, HCDR2 comprises SEQ ID NO: 4. In some embodiments, HCDR2 comprises SEQ ID NO: 5. In some embodiments, HCDR3 comprises SEQ ID NO: 6. In some embodiments, HCDR3 comprises SEQ ID NO: 7. In some embodiments, HCDR3 comprises SEQ ID NO: 8. In some embodiments, HCDR3 comprises SEQ ID NO: 9. In some embodiments, LCDR1 comprises SEQ ID NO: 10. In some embodiments, LCDR2 comprises SEQ ID NO: 11. In some embodiments, LCDR3 comprises SEQ ID NO: 12. In some embodiments, LCDR3 comprises SEQ ID NO: 13. In some embodiments, LCDR3 comprises SEQ ID NO: 14. In some embodiments, the antibody or antigen binding fragment comprises a light chain CDR3 as set forth by SEQ ID NO: 15.

In another aspect, provided herein is an antibody or antigen binding fragment thereof that binds to TL1A, comprising: (a) a heavy chain variable region comprising SEQ ID NO: 302 (XIVQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), and (b) a light chain variable region comprising SEQ ID NO: 303 (EIVLTQSPGTLSLSPGERATLSC[LCDR1]WYQQKPGQAPRX10X11IY[LCDR2]GIPDRES GSGSGTDFTLTISRLEPEDFAVYYC[LCDR3]FGGGTKLEIK), wherein each of X1-X11 is independently selected from A, R, N, D, C, Q, E, G, H, I, L. K, M, F, P, S, T, W, Y, or V.

In some embodiments, X1 comprises a hydrophobic amino acid, a hydrophilic amino acid, or an amphipathic amino acid. In some embodiments, X1 comprises an aliphatic amino acid, an aromatic amino acid, an acidic amino acid, a basic amino acid, a hydroxylic amino acid, a sulfur-containing amino acid, or an amidic amino acid. In some embodiments, X2 comprises a hydrophobic amino acid, a hydrophilic amino acid, or an amphipathic amino acid. In some embodiments, X2 comprises an aliphatic amino acid, an aromatic amino acid, an acidic amino acid, a basic amino acid, a hydroxylic amino acid, a sulfur-containing amino acid, or an amidic amino acid. In some embodiments. X3 comprises a hydrophobic amino acid, a hydrophilic amino acid, or an amphipathic amino acid. In some embodiments, X3 comprises an aliphatic amino acid, an aromatic amino acid, an acidic amino acid, a basic amino acid, a hydroxylic amino acid, a sulfur-containing amino acid, or an amidic amino acid. In some embodiments, X4 comprises a hydrophobic amino acid, a hydrophilic amino acid, or an amphipathic amino acid. In some embodiments, X4 comprises an aliphatic amino acid, an aromatic amino acid, an acidic amino acid, a basic amino acid, a hydroxylic amino acid, a sulfur-containing amino acid, or an amidic amino acid. In some embodiments, X5 comprises a hydrophobic amino acid, a hydrophilic amino acid, or an amphipathic amino acid. In some embodiments, X5 comprises an aliphatic amino acid, an aromatic amino acid, an acidic amino acid, a basic amino acid, a hydroxylic amino acid, a sulfur-containing amino acid, or an amidic amino acid. In some embodiments, X6 comprises a hydrophobic amino acid, a hydrophilic amino acid, or an amphipathic amino acid. In some embodiments, X6 comprises an aliphatic amino acid, an aromatic amino acid, an acidic amino acid, a basic amino acid, a hydroxylic amino acid, a sulfur-containing amino acid, or an amidic amino acid. In some embodiments, X7 comprises a hydrophobic amino acid, a hydrophilic amino acid, or an amphipathic amino acid. In some embodiments, X7 comprises an aliphatic amino acid, an aromatic amino acid, an acidic amino acid, a basic amino acid, a hydroxylic amino acid, a sulfur-containing amino acid, or an amidic amino acid. In some embodiments, X8 comprises a hydrophobic amino acid, a hydrophilic amino acid, or an amphipathic amino acid. In some embodiments, X8 comprises an aliphatic amino acid, an aromatic amino acid, an acidic amino acid, a basic amino acid, a hydroxylic amino acid, a sulfur-containing amino acid, or an amidic amino acid. In some embodiments, X9 comprises a hydrophobic amino acid, a hydrophilic amino acid, or an amphipathic amino acid. In some embodiments, X9 comprises an aliphatic amino acid, an aromatic amino acid, an acidic amino acid, a basic amino acid, a hydroxylic amino acid, a sulfur-containing amino acid, or an amidic amino acid. In some embodiments, X10 comprises a hydrophobic amino acid, a hydrophilic amino acid, or an amphipathic amino acid. In some embodiments, X10 comprises an aliphatic amino acid, an aromatic amino acid, an acidic amino acid, a basic amino acid, a hydroxylic amino acid, a sulfur-containing amino acid, or an amidic amino acid. In some embodiments, X11 comprises a hydrophobic amino acid, a hydrophilic amino acid, or an amphipathic amino acid. In some embodiments, X11 comprises an aliphatic amino acid, an aromatic amino acid, an acidic amino acid, a basic amino acid, a hydroxylic amino acid, a sulfur-containing amino acid, or an amidic amino acid.

In some embodiments, X1=Q or E, X2=R or K, X3=A or R. X4=M or I, X5=V or A, X6=M or I, X7=R or T, X8=V or A, X9=M or L, X10=L or P, and X11=L or W. In some embodiments, X1=Q. In some embodiments, X1=E. In some embodiments, X2=R. In some embodiments, X2=K. In some embodiments, X3=A. In some embodiments, X3=R. In some embodiments, X4=M. In some embodiments, X4=I. In some embodiments, X5=V. In some embodiments, X5=A. In some embodiments, X6=M. In some embodiments, X6=I. In some embodiments, X7=R. In some embodiments, X7=T. In some embodiments, X8=V. In some embodiments, X8=A. In some embodiments, X9=M. In some embodiments, X9=L. In some embodiments, X10=L. In some embodiments, X10=P. In some embodiments, X11=L. In some embodiments, X11=W.

In some embodiments, HCDR1 comprises SEQ ID NO: 1. In some embodiments. HCDR2 comprises SEQ ID NO: 2. In some embodiments, HCDR2 comprises SEQ ID NO: 3. In some embodiments, HCDR2 comprises SEQ ID NO: 4. In some embodiments, HCDR2 comprises SEQ ID NO: 5. In some embodiments, HCDR3 comprises SEQ ID NO: 6. In some embodiments, HCDR3 comprises SEQ ID NO: 7. In some embodiments, HCDR3 comprises SEQ ID NO: 8. In some embodiments, HCDR3 comprises SEQ ID NO: 9. In some embodiments, LCDR1 comprises SEQ ID NO: 10. In some embodiments, LCDR2 comprises SEQ ID NO: 11. In some embodiments, LCDR3 comprises SEQ ID NO: 12. In some embodiments, LCDR3 comprises SEQ ID NO: 13. In some embodiments, LCDR3 comprises SEQ ID NO: 14. In some embodiments, LCDR3 comprises SEQ ID NO: 15.

Further provided herein is an antibody or antigen binding fragment thereof that binds to tumor necrosis factor-like protein 1A (TL1A), comprising a heavy chain variable domain comprising an amino acid sequence at least about 96% identical to SEQ ID NO: 104, and a light chain variable domain comprising an amino acid sequence at least about 97% identical to SEQ ID NO: 201. In some embodiments, the heavy chain variable domain comprises an amino acid sequence at least about 97% identical to SEQ ID NO: 104. In some embodiments, the heavy chain variable domain comprises an amino acid sequence at least about 98% identical to SEQ ID NO: 104. In some embodiments, the heavy chain variable domain comprises an amino acid sequence at least about 99% identical to SEQ ID NO: 104. In some embodiments, the heavy chain variable domain comprises SEQ ID NO: 104. In some embodiments, the light chain variable domain comprises an amino acid sequence at least about 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

Further provided herein is an antibody or antigen binding fragment thereof that binds to tumor necrosis factor-like protein 1A (TL1A), comprising a heavy chain variable domain comprising an amino acid sequence at least about 99% identical to SEQ ID NO: 107, and a light chain variable domain comprising an amino acid sequence at least about 97% identical to SEQ ID NO: 201. In some embodiments, the heavy chain variable domain comprises SEQ ID NO: 107. In some embodiments, the light chain variable domain comprises an amino acid sequence at least about 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

Further provided herein is an antibody or antigen binding fragment thereof that binds to tumor necrosis factor-like protein 1A (TL1A), comprising a heavy chain variable domain comprising SEQ ID NO: 101, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 102, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 103, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 104, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 105, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 103, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 106, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 107, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 109, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 109, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 203. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 107, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 107, and a light chain variable domain comprising SEQ ID NOS: 202. Further provided herein is an antibody or antigen binding fragment thereof that binds to tumor necrosis factor-like protein LA (TL1A), comprising a heavy chain variable domain comprising SEQ ID NO: 110, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 111, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 112, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 113, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 114, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 115, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 116, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 117, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 118, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 114, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 102, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 104, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 119, and a light chain variable domain comprising SEQ ID NOS: 204. Further provided herein is an antibody or antigen binding fragment thereof that binds to tumor necrosis factor-like protein LA (TL1A), comprising a heavy chain variable domain comprising SEQ ID NO: 119, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 101, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 105, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 120, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 121, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 123, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 124, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 125, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 116, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 117, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 126, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 127, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 127, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 121, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 206. Further provided herein is an antibody or antigen binding fragment thereof that binds to tumor necrosis factor-like protein 1A (TL1A), comprising a heavy chain variable domain comprising SEQ ID NO: 124, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 124, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 128, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 128, and a light chain variable domain comprising SEQ ID NOS: 206. Further provided herein is an antibody or antigen binding fragment thereof that binds to tumor necrosis factor-like protein 1A (TL1A), comprising a heavy chain variable domain comprising SEQ ID NO: 129, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 130, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 131, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 132, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 133, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 134, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 135, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 132, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 126, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 130, and a light chain variable domain comprising SEQ ID NOS: 201.

Further provided herein is an antibody or antigen binding fragment thereof that binds to tumor necrosis factor-like protein 1A (TL1A), comprising a heavy chain domain comprising any one of SEQ ID NOs: 501-513, and a light chain domain comprising SEQ ID NOS: 514.

In another aspect, provided herein is an antibody or antigen binding fragment thereof that binds to TL1A, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; (f) an LCDR3 comprising an amino acid sequence set forth by any one of SEQ ID NOS: 12-15; and a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1. In some embodiments, the human IgG1 comprises SEQ ID NO: 320. In some embodiments, the ADCC function of the Fc region comprising reduced ADCC is at least about 50% reduced as compared to human IgG1. In some embodiments, the CDC function of the Fc region comprising reduced ADCC is at least about 50% reduced as compared to human IgG1. In some embodiments, the Fc region comprises a human IgG1 comprising (a) 297A, 297Q, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301 W, (cc) 304E, (dd) 311E, 311G, or 311S, (ee) 316F, (ff) 328V, (gg) 330R, (hh) 339E or 339L. (ii) 3431 or 343V, (jj) 373A, 373G, or 3738, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (III) A330L, (mmm) P331A or P331S, or (nnn) any combination of (a)-(uu), per Kabat numbering. In some embodiments, the antibody of antigen binding fragment comprises a (i) human IgG4 Fc region or (ii) a human IgG4 Fc region comprising (a) S228P and L235E, or (b) S228P. F234A, and L235A, per Kabat numbering. In some embodiments, the antibody of antigen binding fragment comprises a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). In some embodiments, the antibody of antigen binding fragment comprises a human Fe region comprising high mannose glycosylation. In some embodiments, the Fc region comprises a human IgG1 with a substitution selected from 297A, 297Q, 297D, 279F, 279K, 279L, 228P, 235A, 235E, 235G, 235Q, 235R, 235S, 237A, 237E, 237K, 237N, 237R, 268K, 269N, 269Q, 270A, 270G, 270M, 270N, 424H, 424M, and 424V, per Kabat numbering. In some embodiments, the Fc region comprises a human IgG1 with a substitution selected from 271T, 272N, 292E, 292F, 292G, 292I, 293S, 301 W, 304E, 311E, 311G, 311S, 255N, 256H, 256K, 256R, 256V, 316F, 328V, 330R, 339E, 339L, 343I, 343V, 373A, 373G, 373S, 376E, 376 W, 376Y, 380D, 382D, 382P, 385P, 234A, 234V, 234F, 233P, 328A, 327Q and 327T, per Kabat numbering. In some embodiments, the Fc region comprises a human IgG1 with a substitution selected from 329A, 329G, 329Y, 3318, 236F, 236R, 238A, 238E, 238G, 238H, 238I, 238V, 238 W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 4341, 438G, 439E, 439H, 439Q, 440A, 440D, 440E, 440F, 440M, 440T, and 440V, per Kabat numbering. In some embodiments, the antibody or antigen binding fragment comprises a sequence at least about 90%, 91%, 92%, 93%, 94%, 95%, 96%, 97%, 98%, 99%, or 100% identical to any one of SEQ ID NOS: 320-362. In some embodiments, HCDR1 comprises SEQ ID NO: 1. In some embodiments, HCDR2 comprises SEQ ID NO: 2. In some embodiments, HCDR2 comprises SEQ ID NO: 3. In some embodiments, HCDR2 comprises SEQ ID NO: 4. In some embodiments, HCDR2 comprises SEQ ID NO: 5. In some embodiments, HCDR3 comprises SEQ ID NO: 6. In some embodiments, HCDR3 comprises SEQ ID NO: 7. In some embodiments, HCDR3 comprises SEQ ID NO: 8. In some embodiments, HCDR3 comprises SEQ ID NO: 9. In some embodiments, LCDR1 comprises SEQ ID NO: 10. In some embodiments, LCDR2 comprises SEQ ID NO: 11. In some embodiments, LCDR3 comprises SEQ ID NO: 12. In some embodiments, LCDR3 comprises SEQ ID NO: 13. In some embodiments, LCDR3 comprises SEQ ID NO: 14. In some embodiments, LCDR3 comprises SEQ ID NO: 15.

In another aspect, provided herein is an antibody or antigen binding fragment thereof that binds to TL1A, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15. In some embodiments, the heavy chain variable region comprises human IGHV1-46*02 framework or a modified human IGHV1-46*02 framework. In some embodiments, the light chain variable framework region comprising a human IGKV3-20 framework or a modified human IGKV3-20 framework. In some embodiments, the heavy chain variable region comprises one of more of the following amino acids: 1E, 45K, 47R, 551, 78A, 801, 82T, 89A, 91L, per Aho or Kabat numbering. In some embodiments, the heavy chain variable region comprises 1E. In some embodiments, the heavy chain variable region comprises 45K. In some embodiments, the heavy chain variable region comprises 47R. In some embodiments, the heavy chain variable region comprises 551. In some embodiments, the heavy chain variable region comprises 78A. In some embodiments, the heavy chain variable region comprises 801. In some embodiments, the heavy chain variable region comprises 82T. In some embodiments, the heavy chain variable region comprises 89A. In some embodiments, the heavy chain variable region comprises 91L. In some embodiments, the light chain variable region comprises one or more of the following amino acids: 54P and 55 W, per Aho or Kabat numbering. In some embodiments, the light chain variable region comprises 54P. In some embodiments, the light chain variable region comprises 55 W. In some embodiments, the HCDR2 comprises SEQ ID NO: 2. In some embodiments, the HCDR2 comprises SEQ ID NO: 3. In some embodiments, the HCDR2 comprises SEQ ID NO: 4. In some embodiments, the HCDR2 comprises SEQ ID NO: 5. In some embodiments, the HCDR3 comprises SEQ ID NO: 6. In some embodiments, the HCDR3 comprises SEQ ID NO: 7. In some embodiments, the HCDR3 comprises SEQ ID NO: 8. In some embodiments, the HCDR3 comprises SEQ ID NO: 9. In some embodiments, the LCDR3 comprises SEQ ID NO: 12. In some embodiments, the LCDR3 comprises SEQ ID NO: 13. In some embodiments, the LCDR3 comprises SEQ ID NO: 14. In some embodiments, LCDR3 comprises SEQ ID NO: 15.

In some embodiments, an antibody or antigen binding fragment described herein comprises a human IgG1 Fc region comprising (a) 297A, 297Q, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, (k) 331S. (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 27IT, (y) 272N, (z) 292E, 292F, 292G, or 292I, (aa) 293S, (bb) 301 W, (cc) 304E, (dd) 311E, 311G, or 311S, (ec) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P. (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (ece) G237A, (fff) F241A, (ggg) V264A, (hhh) D265A, (iii) D265A and N297A. (jjj) D265A and N297G, (kkk) D270A, (III) A330L, (mmm) P331A or P331S, or (nnn) any combination of (a)-(uu), per Kabat numbering. In some embodiments, an antibody of antigen binding fragment herein comprises a (i) human IgG4 Fc region or (ii) a human IgG4 Fc region comprising (a) S228P and L235E, or (b) S228P, F234A, and L235A, per Kabat numbering. In some embodiments, an antibody of antigen binding fragment herein comprises a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). In some embodiments, an antibody of antigen binding fragment herein comprises a human Fe region comprising high mannose glycosylation. In some embodiments, any of the antibody or antigen binding fragments described herein comprise a human IgG4 Fc region.

In some embodiments, an antibody or antigen binding fragment described herein comprises a human IgG1 with a substitution selected from 297A, 297Q, 297D, 279F, 279K, 279L, 228P, 235A, 235E, 235G, 235Q, 235R, 235S, 237A, 237E, 237K, 237N, 237R, 268K, 269N, 269Q, 270A, 270G, 270M, 270N, 424H, 424M, and 424V, per Kabat numbering. In some embodiments, an antibody or antigen binding fragment described herein comprises a human IgG1 with a substitution selected from 27IT, 272N, 292E, 292F, 292G, 292I, 293S, 301 W, 304E, 311E, 311G, 311S, 255N, 256H, 256K, 256R, 256V, 316F, 328V, 330R, 339E, 339L, 3431, 343V, 373A, 373G, 373S, 376E, 376 W, 376Y, 380D, 382D, 382P, 385P, 234A, 234V, 234F, 233P, 328A, 327Q and 327T, per Kabat numbering. In some embodiments, an antibody or antigen binding fragment described herein comprises a human IgG1 with a substitution selected from 329A, 329G, 329Y, 331S, 236F, 236R, 238A, 238E, 238G, 238H, 238I, 238V, 238 W, 238Y, 248A, 254D, 254E, 254G, 254H, 254I, 254N, 254P, 254Q, 254T, 254V, 264S, 26SH, 265K, 265S, 265Y, 265A, 267G, 267H, 267I, 267K, 4341, 438G, 439E, 439H, 439Q, 440A, 440D, 440E, 440F, 440M, 440T, and 440V, per Kabat numbering.

In some embodiments, an antibody or antigen binding fragment described herein comprises a Fc region comprising a sequence at least about 90%, 91%, 92%, 93%, 94%, 95%, 96%, 97%, 98%, 99%, or 100% identical to any one of SEQ ID NOS: 320-362.

In some embodiments, an antibody or antigen binding fragment described herein comprises at least about 80% monomeric fraction as determined by size exclusion chromatography. In some embodiments, an antibody or antigen binding fragment described herein comprises at least about 85%, 90%, 91%, 92%, 93%, 94%, 95%, 96%, 97%, 98%, or 99% monomeric fraction. In some embodiments, the size exclusion chromatography comprises injecting purified antibody or antigen binding fragment onto a size exclusion column, wherein the antibody or antigen binding fragment is purified by protein A. In some embodiments, the antibody or antigen binding fragment is purified as described in Example 2. In some embodiments, the antibody or antigen binding fragment is expressed under conditions described in Example 2. In some embodiments, the size exclusion chromatography column has an inner diameter of 4.6 mm. In some embodiments, the size exclusion chromatography column has a length of 150 mm. In some embodiments, the size exclusion chromatography column has a pore size of 200 Å. In some embodiments, the size exclusion chromatography column has a particle size of 1.7 micrometer. In some embodiments, the size exclusion chromatography column is ACQUITY UPLC BEH200 SEC column. In some embodiments, the antibody or antigen binding fragment is injected at a total volume of 15 AL. In some embodiments, the antibody or antigen binding fragment is injected at a concentration of about 0.1 μg/μL to about 1.0 μg/μL. In some embodiments, the size exclusion chromatography is performed on a Shimadzu UPLC instrument. In some embodiments, the size exclusion chromatography is performed at a flow rate of 0.2 mL/min. In some embodiments, the size exclusion chromatography is performed at a column oven temperature of 30° C. In some embodiments, the percentage of monomer is calculated using Shimadzu software. In some embodiments, the size exclusion chromatography is performed as described in Example 2.

In some embodiments, an antibody or antigen binding fragment described herein expresses at least about 20 μg/ml total antibody. In some embodiments, an antibody or antigen binding fragment described herein expresses between about 20 μg/ml and 70 μg/mL total antibody. In some embodiments, the antibody or antigen binding fragment is expressed in FreeStyle 293-F cells. In some embodiments, the antibody or antigen binding fragment is expressed as described in Example 2. In some embodiments, the antibody or antigen binding fragment expression level is quantified using Enzyme-Linked Immunosorbent assay (ELISA). In some embodiments, the ELISA comprises coating a surface of a substrate with a capture antibody that binds to a human or humanized antibody, applying the antibody or antigen binding fragment to the substrate, and applying to the substrate a second antibody that binds to a human or humanized antibody. In some embodiments, the capture antibody comprises an anti-kappa antibody. In some embodiments, the second antibody comprises an anti-Fc antibody. In some embodiments, the ELISA is performed as described in Example 2.

Further provided herein are methods of treating inflammatory bowel disease (IBD) in a subject in need thereof, the method comprising administering to the subject an antibody or antigen binding fragment described herein. In some embodiments, the IBD comprises Crohn's Disease. In some embodiments, the IBD comprises ulcerative colitis.

Also provided are antibodies or antigen binding fragments thereof that bind to tumor necrosis factor-like protein 1A (TL1A), comprising a heavy chain variable domain comprising an amino acid sequence at least 96% identical to SEQ ID NO: 104, and a light chain variable domain comprising an amino acid sequence at least 97% identical to SEQ ID NO: 201. In some embodiments, the heavy chain variable domain comprises an amino acid sequence at least 97% identical to SEQ ID NO: 104. In some embodiments, the heavy chain variable domain comprises an amino acid sequence at least 98% identical to SEQ ID NO: 104. In some embodiments, the heavy chain variable domain comprises an amino acid sequence at least 99% identical to SEQ ID NO: 104. In some embodiments, the heavy chain variable domain comprises SEQ ID NO: 104. In some embodiments, the light chain variable domain comprises an amino acid sequence at least 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

Further provided are antibodies or antigen binding fragments thereof that bind to tumor necrosis factor-like protein 1A (TL1A), comprising a heavy chain variable domain comprising an amino acid sequence at least about 99% identical to any one of SEQ ID NOS: 101-135, and a light chain variable domain comprising an amino acid sequence at least about 99% identical to any one of SEQ ID NOS: 201-206. Table 16 and Table 17 set forth exemplary variable region amino acid sequences of anti-TL1A antibodies.

Provided are also antibodies or antigen binding fragments, as described herein, further comprising a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (1) 329A, 329G, 329Y, or 329R (k) 331S, (1) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 2671, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376 W, or 376Y, (II) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L23SA, G237A, P238S, H268A, A330S, and P331S (IgGlo, (ccc) L234A, L235A, and P329A, (ddd) G236R and L328R, (eee) G237A, (fff) F241A, (ggg) V264A, (hhh) D265A, (iii) D265A and N297A, (jj) D265A and N297G, (kkk) D270A, (III) A330L, (mmm) P331A or P331S, or (nnn) any combination of (a)-(uu), per Kabat numbering. In some embodiments, the antibodies comprise a human IgG4 Fc region. In some embodiments, the antibodies a Fc region comprising a sequence at least about 90%, 91%, 92%, 93%, 94%, 95%, 96%, 97%, 98%, 99%, or 100% identical to any one of SEQ ID NOS: 320-362. In some embodiments, the antibodies comprise at least about 80% monomeric fraction as determined by size exclusion chromatography. In some embodiments, the antibodies are expressed in an amount at least about 20 μg/ml total antibody, optionally about 20 μg/ml and 70 μg/mL total antibody.

### 5.3.1.6 Antibody Properties

In various embodiments, the anti-TL1A antibody is an antagonist of a TL1A receptor, such as, but not limited to, DR3 and TR6/DcR3. In certain embodiments, the antibody inhibits at least about 10%, at least about 20%, at least about 30%, at least about 50%, at least about 75%, at least about 90%, or about 100% of one or more activity of the bound TL1A receptor. In certain embodiments, the antibodies inhibit TL1A activation as measured by interferon gamma release in human blood. In certain embodiments, the antibody inhibits interferon gamma release in human blood at an IC₅₀ of between about 1 nanomolar and about 30 picomolar. In certain embodiments, the antibody inhibits interferon gamma release in human blood at an IC₅₀ of between about 500 picomolar and about 30 picomolar. In certain embodiments, the antibody inhibits interferon gamma release in human blood at an IC₅₀ of between about 200 picomolar and about 30 picomolar. In certain embodiments, the antibody inhibits interferon gamma release in human blood at an IC₅₀ of less than or equal to about 200 picomolar. In certain embodiments, the antibody inhibits interferon gamma release in human blood at an IC₅₀ of less than or equal to about 100 picomolar.

In various embodiments, an anti-TL1A antibody provided herein has a binding affinity to human TL1A of less than about 1E⁻⁷, 1E⁻⁸, 1E⁻⁹, or 1E⁻¹⁰ Kd. In some cases, the binding affinity is from about 1E⁻⁹ to about 1E⁻¹⁰ Kd. In some embodiments, an anti-TL1A antibody provided herein has a binding affinity to murine TL1A and/or rat TL1A of less than about 1E-7, 1E⁻⁸, 1E⁻⁹, 1E⁻¹⁰, or 1E⁻¹¹ Kd. Methods for determining binding affinity are exemplified herein, including in Example 2.

In various embodiments, an anti-TL1A antibody provided herein comprises at least about 80% monomeric fraction after expression and purification as described in Example 2 or elsewhere herein. In various embodiments, an anti-TL1A antibody provided herein comprises at least about 85% monomeric fraction after expression and purification as described in Example 2 or elsewhere herein. In various embodiments, an anti-TL1A antibody provided herein comprises at least about 90% monomeric fraction after expression and purification as described in Example 2 or elsewhere herein. In various embodiments, an anti-TL1A antibody provided herein comprises at least about 91%, 92%, 93%, 94%, 95%, 96%, 97%, 98%, 99%, or 100% monomeric fraction after expression and purification as described in Example 2 or elsewhere herein.

In various embodiments, an anti-TL1A antibody provided herein has at least about 2 μg/mL expression as determined by the method disclosed herein. In some embodiments, the anti-TL1A antibody has about 2 μg/mL to about 60 μg/mL expression as determined by the method disclosed herein. In some embodiments, the anti-TL1A antibody has about 5 μg/mL to about 60 μg/mL expression as determined by the method disclosed herein. In some embodiments, the anti-TL1A antibody has about 10 μg/mL to about 60 μg/mL expression as determined by the method disclosed herein. In some embodiments, the anti-TL1A antibody has at least about 5 μg/mL expression as determined by the method disclosed herein. In some embodiments, the anti-TL1A antibody has at least about 10 μg/mL expression as determined by the method disclosed herein. In some embodiments, the anti-TL1A antibody has at least about 15 μg/mL expression as determined by the method disclosed herein. In some embodiments, the anti-TL1A antibody has at least about 20 g/mL expression as determined by the method disclosed herein. In some embodiments, the anti-TL1A antibody expresses between about 2 μg/mL and about 50 μg/mL, between about 2 μg/mL and about 40 μg/mL, between about 2 μg/mL and about 30 ng/ml expression, between about 2 μg/mL and about 20 μg/mL, between about 5 μg/mL and about 50 μg/mL, between about 5 μg/mL and about 40 g/mL, between about 5 μg/mL and about 30 μg/mL, between about 10 μg/mL and about 50 μg/mL, between about 10 μg/mL and about 40 μg/mL, or between about 10 μg/mL and about 30 μg/mL as determined by the method disclosed herein. In some embodiments, the anti-TL1A antibody has about 2, 3, 4, 5, 6, 7, 8, 9, 10, 11, 12, 13, 14, 15, 16, 17, 18, 19, 20, 21, 22, 23, 24, 25, 26, 27, 28, 29, 30 μg/mL expression as determined by the method disclosed herein. Methods disclosed herein include those described in Example 2.

In various embodiments, an anti-TL1A antibody provided herein is humanized and has less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. The humanized heavy chain variable domain may comprise IGHV1-46*02 framework with no or fewer than about 10, 9, 8, 7, 6, 5, 4, 3, 2, or I non-human mutations, The humanized light chain variable domain may comprise IGKV3-20 framework with no or fewer than about 10, 9, 8, 7, 6, 5, 4, 3, 2, or 1 non-human mutations.

### 5.3.2 Assays

An exemplary screening paradigm for identification of antibody variants that express well in mammalian cells and preserve TL1A binding activity while minimizing the propensity of the antibody to aggregate comprises a five-step process. This screen was performed as detailed in the examples. Briefly, (1) variants were cloned and transiently expressed as intact Ig in 293 cells using small-scale (3 mL, 6-well culture plates) transfections, (2) the expression level of the antibody was assessed in the culture supernatant 96-120 hours after transfection using an antibody quantitation ELISA, (3) the binding of the supernatant antibody variants to human TL1A was assessed by ELISA, (4) the antibody was purified in a single step using Protein A and (5) the material was analyzed by analytical SEC to assess monomer/aggregate content. This approach enabled identification of variants that expressed well, preserved binding to TL1A, and displayed high monomer content.

Further provided herein are methods for analyzing antibody solubility based on percentage of monomeric fraction. For example, as described in Example 2.

Further provided herein are assays for quantifying antibody expression. For example, as described in Example 2.

Further provided herein are assays for quantifying immunogenicity of an antibody.

The antibodies described herein can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are provided in for e.g., Ausubel et al., eds, 1994, Current Protocols in Molecular Biology, Vol. 1, John Wiley & Sons, Inc., New York.

### 5.3.3 Additional Therapeutic Agent

A therapeutic agent may be used alone or in combination with an additional therapeutic agent. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

### 5.3.4 Methods of Generating Antibodies

In various embodiments, monoclonal antibodies are prepared using methods known in the art, such as, but not limited to the hybridoma method, where a host animal is immunized to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid by techniques known in the art, when propagated either in vitro or in vivo.

In some embodiments, monoclonal antibodies are made using recombinant DNA methods. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, a chimeric antibody, a molecule in which different portions are derived from different animal species, such as those having a variable region derived from a murine monoclonal antibody and a human immunoglobulin constant region (e.g., humanized antibodies) can be generated.

In some embodiments, the anti-TL LA monoclonal antibody is a humanized antibody, to reduce antigenicity and HAMA (human anti-mouse antibody) responses when administered to a human subject. Humanized antibodies can be produced using various techniques known in the art. For example, an antibody is humanized by (1) determining the nucleotide and predicted amino acid sequence of the starting antibody light and heavy variable domains: (2) designing the humanized antibody, e.g., deciding which antibody framework region to use during the humanizing process: (3) the actual humanizing methodologies/techniques; and (4) the transfection and expression of the humanized antibody. In various embodiments, a humanized antibody can be further optimized to decrease potential immunogenicity, while maintaining functional activity, for therapy in humans.

Humanized antibodies can also be made in transgenic mice containing human immunoglobulin loci that are capable, upon immunization, of producing the full repertoire of human antibodies in the absence of endogenous immunoglobulin production. A humanized antibody may also be obtained by a genetic engineering approach that enables production of affinity-matured human-like polyclonal antibodies in large animals.

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications should therefore be restricted to a minimum and closely watched for changes in the specificity and affinity of the antibody. On the other hand, framework residues in theory can be derived from any human variable region. A human framework sequences should be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g., mutated or affinity matured) sequences. Genetic engineering techniques well known to those in the art, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR.

In certain embodiments, the anti-TL1A antibody is a human antibody. Human antibodies can be directly prepared using various techniques known in the art. Immortalized human B lymphocytes immunized in vitro or isolated from an immunized individual that produce an antibody directed against a target antigen can be generated.

Chimeric, humanized and human antibodies may be produced by recombinant expression. Recombinant polynucleotide constructs typically include an expression control sequence operably linked to the coding sequences of antibody chains, including naturally associated or heterologous promoter regions. In certain embodiments, it may be desirable to generate amino acid sequence variants of these humanized antibodies, particularly where these improve the binding affinity or other biological properties of the antibody.

In certain embodiments, an antibody fragment is used to treat and/or ameliorate IBD. Various techniques are known for the production of antibody fragments. Generally, these fragments are derived via proteolytic digestion of intact antibodies (for example Morimoto et al., 1993, Journal of Biochemical and Biophysical Methods 24:107-117; Brennan et al., 1985, Science, 229:81). Fab, Fv, and scFv antibody fragments can all be expressed in and secreted from E. coli or other host cells, thus allowing the production of large amounts of these fragments. Other techniques for the production of antibody fragments will be apparent to the skilled practitioner.

According to the present disclosure, techniques can be adapted for the production of single-chain antibodies specific to TL1A. In addition, methods can be adapted for the construction of Fab expression libraries to allow rapid and effective identification of monoclonal Fab fragments with the desired specificity for TL1A, or derivatives, fragments, analogs or homologs thereof. Antibody fragments may be produced by techniques in the art including, but not limited to: (a) a F (ab)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

Also provided herein are modified antibodies comprising any type of variable region that provides for the association of the antibody with TL1A. Those skilled in the art will appreciate that the modified antibodies may comprise antibodies (e.g., full-length antibodies or immunoreactive fragments thereof) in which at least a fraction of one or more of the constant region domains has been deleted or otherwise altered so as to provide desired biochemical characteristics such as decreasing TL1A. In certain embodiments, the variable regions in both the heavy and light chains are altered by at least partial replacement of one or more CDRs and, if necessary, by partial framework region replacement and sequence changing. In some embodiments, the replaced CDRs may be derived from an antibody of the same class, subclass, from an antibody of a different class, for instance, from an antibody from a different species and/or a combination thereof. In some embodiments, the constant region of the modified antibodies will comprise a human constant region. Modifications to the constant region compatible with this disclosure comprise additions, deletions or substitutions of one or more amino acids in one or more domains.

In various embodiments, the expression of an antibody or antigen-binding fragment thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody or antigen-fragment thereof as described herein may be transfected into the host.

In some embodiments, the expression vectors are transfected into the recipient cell line for the production of the chimeric, humanized, or composite human antibodies described herein. In various embodiments, mammalian cells can be useful as hosts for the production of antibody proteins, which can include, but are not limited to cells of fibroblast origin, such as Vero (ATCC CRL 81) or CHO-KI (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO—S and DG44 cells; PER.C6™ cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying a chimeric, humanized, or composite human antibody construct, antibody or antigen-binding fragment thereof as described herein can be introduced into an appropriate host cell by any of a variety of suitable means, depending on the type of cellular host including, but not limited to transformation, transfection, lipofection, conjugation, electroporation, direct microinjection, and microprojectile bombardment, as known to one of ordinary skill in the art. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody molecules or peptides described herein. In various other embodiments, bacterial strains can also be utilized as hosts for the production of the antibody molecules or peptides described herein. Examples of bacterial strains include, but are not limited to E. coli, Bacillus species, enterobacteria, and various Pseudomonas species.

In some embodiments, one or more antibodies or antigen-binding fragments thereof as described herein can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982)).

Once expressed in the host, the whole antibodies, antibody-fragments (e.g., individual light and heavy chains), or other immunoglobulin forms of the present disclosure can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, etc. See generally, Vols. I & II Immunol. Meth. (Lefkovits & Pernis, eds., Acad. Press, NY, 1979 and 1981).

Various embodiments provide for a genetic construct comprising a nucleic acid encoding an anti-TL1A antibody or fragment provided herein. Genetic constructs of the antibody can be in the form of expression cassettes, which can be suitable for expression of the encoded anti-TL1A antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by methods known in the art. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3T3, Chinese hamster ovary (CHO), HeLa and BHK cell lines. Mammalian expression vectors can comprise non-transcribed elements such as an origin of replication, a suitable promoter and enhancer linked to the gene to be expressed, and other S′ or 3′ flanking non-transcribed sequences, and 5′ or 3′ non-translated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine (SEQ ID NO: 1303), maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography. Recombinant protein produced in bacterial culture can be isolated.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp: or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Particular conservative substitutions include, for example; Ala into Gly or into Ser; Arg into Lys: Asn into Gin or into His; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu: Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding fragment thereof described herein can be a variant of a sequence described herein, e.g., a conservative substitution variant of an antibody polypeptide. In some embodiments, the variant is a conservatively modified variant. A variant may refer to a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g., antigen-specific binding activity for the relevant target polypeptide.

Alterations of the native amino acid sequence can be accomplished by any of a number of techniques known to one of skill in the art. Mutations can be introduced at particular loci or by oligonucleotide-directed site-specific mutagenesis procedures. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981).

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, NY, 1982 and 1989), and can be used to construct nucleic acid sequences which encode a monoclonal antibody molecule or antigen-binding region.

In some embodiments, a nucleic acid encoding an antibody or antigen-binding fragment thereof as described herein is comprised by a vector. In some of the aspects described herein, a nucleic acid sequence encoding an antibody or antigen-binding fragment thereof as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (S′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding portion thereof as described herein in place of non-essential viral genes. The vector and/or particle may be utilized for the purpose of transferring any nucleic acids into cells either in vitro or in vivo. Numerous forms of viral vectors are known in the art.

By “recombinant vector,” it is meant that the vector includes a heterologous nucleic acid sequence, or “transgene” that is capable of expression in vivo.

### 5,4 Pharmaceutical Compositions, Administration and Dosage

The anti-TL1A antibodies provided are useful in a variety of applications including, but not limited to, therapeutic treatment methods, such as the treatment of IBD. The methods of use may be in vitro, ex vivo, or in vivo methods. In certain embodiments, the anti-TL1A antibody is an antagonist for TL1A receptors.

In certain embodiments, the disease treated with anti-TL1A antibody or TL1A receptor antagonist is IBD, CD, UC and/or MR-UC.

In various embodiments, the pharmaceutical compositions are formulated for delivery via any route of administration. “Route of administration” may refer to any administration pathway known in the art, including but not limited to aerosol, nasal, oral, transmucosal, transdermal or parenteral.

The pharmaceutical compositions can also contain any pharmaceutically acceptable carrier. “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, provided are pharmaceutical compositions including a pharmaceutically acceptable excipient along with a therapeutically effective amount of an anti-TL1A antibody. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain minor amounts of auxiliary substances such as wetting or emulsifying agents, pH buffering agents and the like which enhance or maintain the effectiveness of the active ingredient. Therapeutic compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of an active agent (i.e. antibody or fragment thereof) used that will be effective in the treatment of a particular disorder or condition will depend on the nature of the disorder or condition and can be determined by one of skill in the art with standard clinical techniques.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA)(2000).

For the treatment of the disease, the appropriate dosage of an antibody depends on the type of disease to be treated, the severity and course of the disease, the responsiveness of the disease, whether the antibody is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates. The TL1A antibody can be administered one time or over a series of treatments lasting from several days to several months, or until a cure is effected or a diminution of the disease state is achieved (e.g., treatment or amelioration of IBD). The duration of treatment depends upon the subject's clinical progress and responsiveness to therapy. In certain embodiments, dosage is from 0.01 μg to 100 mg per kg of body weight, and can be given once or more daily, weekly, monthly or yearly.

Antibody therapeutics suitable for injection and/or administration are important to realizing the full therapeutic potential of mAbs (monoclonal antibodies, e.g. an anti-TL1A monoclonal antibody). However, administration is generally restricted by volume. This, in turn, elucidates the importance developing of high concentration mAb formulations of greater than, for example in some cases, 100 mg/ml. Problems associated with mAb development include high solution viscosity and opalescence, which are commonly encountered during the development of high-concentration (e.g. greater than 100 mg/ml) formulation. Both viscosity and opalescence impact mAb developability broadly, affecting manufacturability, stability, and delivery. High solution viscosities (e.g. greater than 30 mPa-s) cause limiting back-pressures in ultrafiltration/diafiltration during the mAb concentration unit operation. Similarly, viscous mAb solutions also result in forbidding or incompatible injection forces when administering via injection, including via patient friendly autoinjectors. In effect, solution viscosity can be a determining factor for the maximum mAb dose possible via injection. Solution opalescence in therapeutic mAbs can be equally problematic as opalescence can indicate predisposition for liquid-liquid phase separation, precipitation, or aggregation.

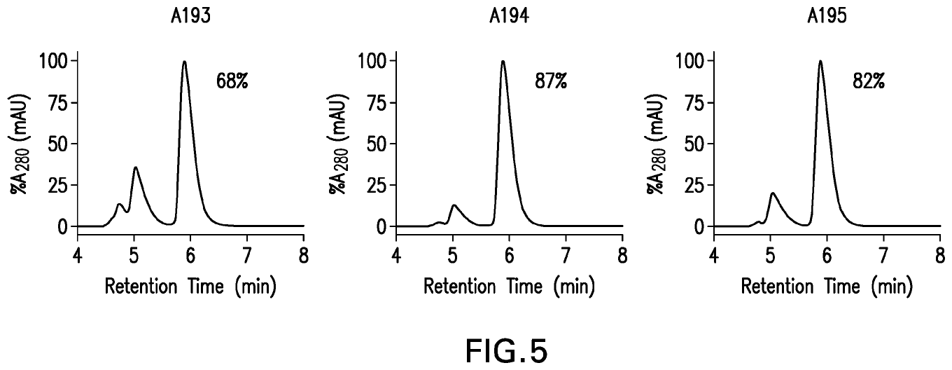

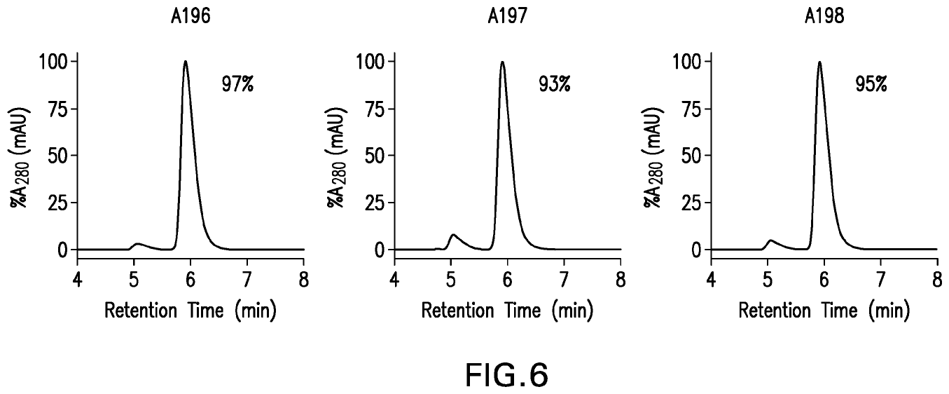

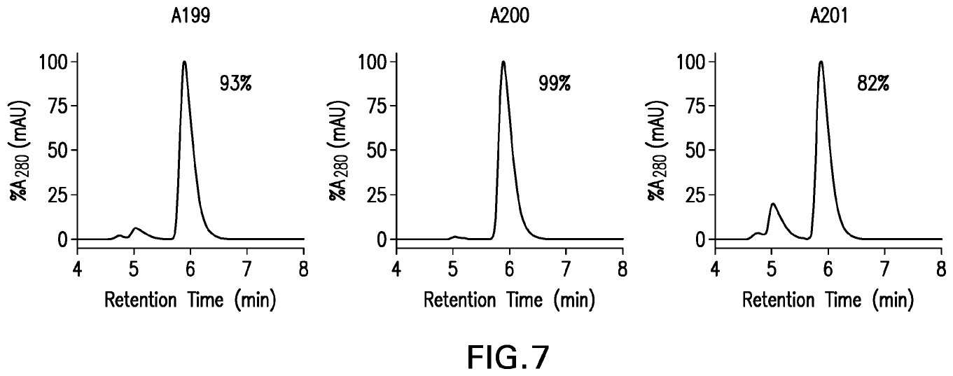

The anti-TL1A antibodies provided herein demonstrate advantageous viscosity and aggregation properties at high antibody concentrations (e.g. greater than 100 mg/mL or greater than 150 mg/mL). Notably, anti-TL1A antibodies provided herein are characterized by low viscosity (e.g. less than 10 mPa-s) and low aggregation (e.g. less than 5% high molecular weight species) at high concentrations (FIGS. 5-7).

For example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-TILA antibody is characterized by a viscosity less than about 30, 20, 15, or 10 mPa-s at a concentration greater than about 100 mg/mL, e.g., up to about 170 mg/mL. In some embodiments, the anti-TILA antibody is characterized by a viscosity less than about 30, 20, 15, or 10 mPa-s at a concentration greater than at least about 100 mg/mL. In some embodiments, the anti-TILA antibody is characterized by a viscosity less than about 30, 20, 15, or 10 mPa-s at a concentration up to about 170 mg/mL. In some embodiments, the anti-T1LA antibody is characterized by a viscosity less than about 30, 20, 15, or 10 mPa-s at a concentration from about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/ml, about 125 mg/ml to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-T1LA antibodies is characterized by a viscosity less than about 30, 20, 15, or 10 mPa-s at a concentration about or greater than about 100 mg/mL, about 125 mg/mL, about 150 mg/mL, about 160 mg/mL, or about 170 mg/mL. In some embodiments, less than about 10 mPa-s includes from about 4 to about 10 mPa-s, from about 4 to about 9 mPa-s, from about 4 to about 8 mPa-s, from about 4 to about 7 mPa-s, from about 4 to about 6 mPa-s, from about 4 to about 5 mPa-s, from about 5 to about 10 mPa-s, from about 5 to about 9 mPa-s, from about 5 to about 8 mPa-s, from about 5 to about 7 mPa-s, from about 5 to about 6 mPa-s, from about 6 to about 10 mPa-s, from about 6 to about 9 mPa-s, from about 6 to about 8 mPa-s, or from about 6 to about 7 mPa-s. In some embodiments, greater than about 100, 125, 150, or 160 mg/ml is up to about 170 mg/ml.

Additionally, for example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-TL1A antibody is characterized by a turbidity less than 12 Nephelometric Turbidity Units (NTU) when at a concentration greater than about 100 mg/mL to about 170 mg/mL. In some embodiments, the anti-TL1A antibody is characterized by a turbidity less than 12 Nephelometric Turbidity Units (NTU) when at a concentration greater than at least about 100 mg/mL. In some embodiments, the anti-TL1A antibody is characterized by a turbidity less than 12 Nephelometric Turbidity Units (NTU) when at a concentration up to about 170 mg/mL. In some embodiments, the anti-TL1A antibody is characterized by a turbidity less than 12 Nephelometric Turbidity Units (NTU) when at a concentration from about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody is characterized by a turbidity less than 12 Nephelometric Turbidity Units (NTU) when at a concentration at or greater than about 100 mg/mL, about 125 mg/mL, about 150 mg/mL, about 160 mg/mL, or about 170 mg/mL.

By way of further example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-T1LA antibody at a concentration from about 150 mg/mL to about or greater than about 170 mg/mL is characterized by a viscosity less than about 10 mPa-s to about 30 mPa-s. In some embodiments, the anti-T1LA antibody at a concentration from about 150 mg/mL to about or greater than about 170 mg/mL is characterized by a viscosity less than about 30 mPa-s. In some embodiments, the anti-T1LA antibody at a concentration from about 150 mg/mL to about or greater than about 170 mg/mL is characterized by a viscosity less than about 5 mPa-s to about 10 mPa-s, about 5 mPa-s to about 15 mPa-s, about 5 mPa-s to about 20 mPa-s, about 5 mPa-s to about 30 mPa-s, about 10 mPa-s to about 15 mPa-s, about 10 mPa-s to about 20 mPa-s, about 10 mPa-s to about 30 mPa-s, about 15 mPa-s to about 20 mPa-s, about 15 mPa-s to about 30 mPa-s, about 20 mPa-s to about 30 mPa-s, about 5 mPa-s to about 9 mPa-s, about 4 to about 10 mPa-s, about 4 to about 9 mPa-s, about 4 to about 8 mPa-s, about 4 to about 7 mPa-s, about 4 to about 6 mPa-s, about 4 to about 5 mPa-s, about 5 to about 10 mPa-s, about 5 to about 9 mPa-s, about 5 to about 8 mPa-s, about 5 to about 7 mPa-s, about 5 to about 6 mPa-s, about 6 to about 10 mPa-s, about 6 to about 9 mPa-s, about 6 to about 8 mPa-s, or about 6 to about 7 mPa-s. In some embodiments, the anti-T1LA antibody at a concentration of about 150 mg/mL to about or greater than about 170 mg/mL is characterized by a viscosity less than about 5 mPa-s, about 10 mPa-s, about 15 mPa-s, about 20 mPa-s, or about 30 mPa-s. In some embodiments, less than about 5, 10, 15, 20, or 30 mPa-s is at least about 1 mPa-s.

Additionally, for example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-TL1A antibody having a concentration greater than 150 mg/mL is characterized by a turbidity less than about 5 Nephelometric Turbidity Units (NTU) to about 15 Nephelometric Turbidity Units (NTU). In some embodiments, the anti-TL1A antibody having a concentration greater than 150 mg/mL is characterized by a turbidity less than at least about 5 Nephelometric Turbidity Units (NTU). In some embodiments, the anti-TL1A antibody having a concentration greater than 150 mg/mL is characterized by a turbidity less than at most about 15 Nephelometric Turbidity Units (NTU). In some embodiments, the anti-TL1A antibody having a concentration greater than 150 mg/mL is characterized by a turbidity less than about 5 Nephelometric Turbidity Units (NTU) to about 7.5 Nephelometric Turbidity Units (NTU), about 5 Nephelometric Turbidity Units (NTU) to about 10 Nephelometric Turbidity Units (NTU), about 5 Nephelometric Turbidity Units (NTU) to about 12.5 Nephelometric Turbidity Units (NTU), about 5 Nephelometric Turbidity Units (NTU) to about 15 Nephelometric Turbidity Units (NTU), about 7.5 Nephelometric Turbidity Units (NTU) to about 10 Nephelometric Turbidity Units (NTU), about 7.5 Nephelometric Turbidity Units (NTU) to about 12.5 Nephelometric Turbidity Units (NTU), about 7.5 Nephelometric Turbidity Units (NTU) to about 15 Nephelometric Turbidity Units (NTU), about 10 Nephelometric Turbidity Units (NTU) to about 12.5 Nephelometric Turbidity Units (NTU), about 10 Nephelometric Turbidity Units (NTU) to about 15 Nephelometric Turbidity Units (NTU), or about 12.5 Nephelometric Turbidity Units (NTU) to about 15 Nephelometric Turbidity Units (NTU). In some embodiments, the anti-TL1A antibody having a concentration greater than 150 mg/mL is characterized by a turbidity less than about 5 Nephelometric Turbidity Units (NTU), about 7.5 Nephelometric Turbidity Units (NTU), about 10 Nephelometric Turbidity Units (NTU), about 12.5 Nephelometric Turbidity Units (NTU), or about 15 Nephelometric Turbidity Units (NTU).

The anti-TL1A antibodies described herein also demonstrate advantageous aggregation properties. For an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-TL1A antibody composition is characterized by percent high molecular weight species (e.g. a species having a molecular weight greater than the molecular weight of the monomer)) less than 10% when at a concentration greater than about 100 mg/mL to about greater than 170 mg/mL. In some embodiments, the anti-TL1A antibody composition is characterized by percent high molecular weight species less than 10% when at a concentration greater than at least about 100 mg/mL. In some embodiments, the anti-TL1A antibody composition is characterized by percent high molecular weight species less than 10% when at a concentration up to about 170 mg/mL. In some embodiments, the anti-TL1A antibody composition is characterized by percent high molecular weight species less than 10% when at a concentration from about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody composition is characterized by percent high molecular weight species less than 10% when at a concentration about or greater than about 100 mg/mL, about 125 mg/mL, about 150 mg/mL, about 160 mg/mL, or about 170 mg/mL. In some embodiments, the anti-TL1A antibody composition having an antibody concentration greater than 150 mg/mL is characterized by a high molecular weight species less than about 5% to about 15%. In some embodiments, the anti-TL1A antibody composition having an antibody concentration greater than 150 mg/mL is characterized by a high molecular weight species less than at most about 15%. In some embodiments, the anti-TL1A antibody composition having an antibody concentration greater than 150 mg/mL is characterized by a high molecular weight species less than about 5% to about 7.5%, about 5% to about 10%, about 5% to about 15%, about 5% to about 17.5%, about 5% to about 20%, about 5% to about 25%, about 7.5% to about 10%, about 7.5% to about 15%, about 7.5% to about 17.5%, about 7.5% to about 20%, about 7.5% to about 25%, about 10% to about 15%, about 10% to about 17.5%, about 10% to about 20%, about 10% to about 25%, about 15% to about 17.5%, about 15% to about 20%, about 15% to about 25%, about 17.5% to about 20%, about 17.5% to about 25%, or about 20% to about 25%. In some embodiments, the anti-TL1A antibody composition having an antibody concentration greater than 150 mg/mL is characterized by a high molecular weight species less than about 5%, about 7.5%, about 10%, about 15%, about 17.5%, about 20%, or about 25%.

By way of further example, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-T1LA antibodies is characterized by a viscosity less than 10 mPa-s at a concentration greater than about 100 mg/mL to about greater than 170 mg/mL. In some embodiments, the anti-T1LA antibodies is characterized by a viscosity less than 10 mPa-s at a concentration greater than at least about 100 mg/mL. In some embodiments, the anti-T1LA antibodies is characterized by a viscosity less than 10 mPa-s at a concentration greater than at most about 170 mg/mL. In some embodiments, the anti-T1LA antibodies is characterized by a viscosity less than 10 mPa-s at a concentration greater than about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-T1LA antibodies is characterized by a viscosity less than 10 mPa-s at a concentration greater than about 100 mg/mL, about 125 mg/mL, about 150 mg/mL, about 160 mg/mL, or about 170 mg/mL.

Additionally, for example, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-TL1A antibody is characterized by a turbidity less than 12 Nephelometric Turbidity Units (NTU) when at a concentration greater than about 100 mg/mL to about greater than 170 mg/mL. In some embodiments, the anti-TL1A antibody is characterized by a turbidity less than 12 Nephelometric Turbidity Units (NTU) when at a concentration greater than at least about 100 mg/mL. In some embodiments, the anti-TL1A antibody is characterized by a turbidity less than 12 Nephelometric Turbidity Units (NTU) when at a concentration greater than at most about 170 mg/mL. In some embodiments, the anti-TL1A antibody is characterized by a turbidity less than 12 Nephelometric Turbidity Units (NTU) when at a concentration greater than about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody is characterized by a turbidity less than 12 Nephelometric Turbidity Units (NTU) when at a concentration greater than about 100 mg/mL, about 125 mg/mL, about 150 mg/mL, about 160 mg/mL, or about 170 mg/mL.

Additionally, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-T1LA antibody at a concentration greater than 150 mg/mL to greater than about 170 mg/mL is characterized by a viscosity less than about 10 mPa-s to about 30 mPa-s. In some embodiments, the anti-T1LA antibody at a concentration greater than 150 mg/mL to greater than about 170 mg/mL is characterized by a viscosity less than at most about 30 mPa-s. In some embodiments, the anti-T1LA antibody at a concentration greater than 150 mg/mL to greater than about 170 mg/mL is characterized by a viscosity less than about 5 mPa-s to about 10 mPa-s, about 5 mPa-s to about 15 mPa-s, about 5 mPa-s to about 20 mPa-s, about 5 mPa-s to about 30 mPa-s, about 10 mPa-s to about 15 mPa-s, about 10 mPa-s to about 20 mPa-s, about 10 mPa-s to about 30 mPa-s, about 15 mPa-s to about 20 mPa-s, about 15 mPa-s to about 30 mPa-s, or about 20 mPa-s to about 30 mPa-s. In some embodiments, the anti-T1LA antibody at a concentration greater than 150 mg/mL to greater than about 170 mg/mL is characterized by a viscosity less than about 5 mPa-s, about 10 mPa-s, about 15 mPa-s, about 20 mPa-s, or about 30 mPa-s.

Additionally, for example, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-TL1A antibody having a concentration greater than 150 mg/mL is characterized by a turbidity less than about 5 Nephelometric Turbidity Units (NTU) to about 15 Nephelometric Turbidity Units (NTU). In some embodiments, the anti-TL1A antibody having a concentration greater than 150 mg/mL is characterized by a turbidity less than at least about 5 Nephelometric Turbidity Units (NTU). In some embodiments, the anti-TL1A antibody having a concentration greater than 150 mg/mL is characterized by a turbidity less than at most about 15 Nephelometric Turbidity Units (NTU). In some embodiments, the anti-TL1A antibody having a concentration greater than 150 mg/mL is characterized by a turbidity less than about 5 Nephelometric Turbidity Units (NTU) to about 7.5 Nephelometric Turbidity Units (NTU), about 5 Nephelometric Turbidity Units (NTU) to about 10 Nephelometric Turbidity Units (NTU), about 5 Nephelometric Turbidity Units (NTU) to about 12.5 Nephelometric Turbidity Units (NTU), about 5 Nephelometric Turbidity Units (NTU) to about 15 Nephelometric Turbidity Units (NTU), about 7.5 Nephelometric Turbidity Units (NTU) to about 10 Nephelometric Turbidity Units (NTU), about 7.5 Nephelometric Turbidity Units (NTU) to about 12.5 Nephelometric Turbidity Units (NTU), about 7.5 Nephelometric Turbidity Units (NTU) to about 15 Nephelometric Turbidity Units (NTU), about 10 Nephelometric Turbidity Units (NTU) to about 12.5 Nephelometric Turbidity Units (NTU), about 10 Nephelometric Turbidity Units (NTU) to about 15 Nephelometric Turbidity Units (NTU), or about 12.5 Nephelometric Turbidity Units (NTU) to about 15 Nephelometric Turbidity Units (NTU). In some embodiments, the anti-TL1A antibody having a concentration greater than 150 mg/mL is characterized by a turbidity less than about 5 Nephelometric Turbidity Units (NTU), about 7.5 Nephelometric Turbidity Units (NTU), about 10 Nephelometric Turbidity Units (NTU), about 12.5 Nephelometric Turbidity Units (NTU), or about 15 Nephelometric Turbidity Units (NTU).

The anti-TL1A antibodies described herein also demonstrate advantageous aggregation properties. For an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-TL1A antibody composition is characterized by percent high molecular weight species (e.g. a species having a molecular weight greater than the molecular weight of the monomer)) less than 10% when at a concentration greater than about 100 mg/mL to about greater than 170 mg/mL. In some embodiments, the anti-TL1A antibody composition is characterized by percent high molecular weight species less than 10% when at a concentration greater than at least about 100 mg/mL. In some embodiments, the anti-TL1A antibody composition is characterized by percent high molecular weight species less than 10% when at a concentration greater than at most about 170 mg/mL. In some embodiments, the anti-TL1A antibody composition is characterized by percent high molecular weight species less than 10% when at a concentration greater than about 100 mg/mL to about 125 mg/ml, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody composition is characterized by percent high molecular weight species less than 10% when at a concentration greater than about 100 mg/mL, about 125 mg/mL, about 150 mg/mL, about 160 mg/mL, or about 170 mg/mL. In some embodiments, the anti-TL1A antibody composition having an antibody concentration greater than 150 mg/mL is characterized by a high molecular weight species less than about 5% to about 15%. In some embodiments, the anti-TL1A antibody composition having an antibody concentration greater than 150 mg/mL is characterized by a high molecular weight species less than at most about 15%. In some embodiments, the anti-TL1A antibody composition having an antibody concentration greater than 150 mg/mL is characterized by a high molecular weight species less than about 5% to about 7.5%, about 5% to about 10%, about 5% to about 15%, about 5% to about 17.5%, about 5% to about 20%, about 5% to about 25%, about 7.5% to about 10%, about 7.5% to about 15%, about 7.5% to about 17.5%, about 7.5% to about 20%, about 7.5% to about 25%, about 10% to about 15%, about 10% to about 17.5%, about 10% to about 20%, about 10% to about 25%, about 15% to about 17.5%, about 15% to about 20%, about 15% to about 25%, about 17.5% to about 20%, about 17.5% to about 25%, or about 20% to about 25%. In some embodiments, the anti-TL1A antibody composition having an antibody concentration greater than 150 mg/mL is characterized by a high molecular weight species less than about 5%, about 7.5%, about 10%, about 15%, about 17.5%, about 20%, or about 25%.

### 5.4.1 Dosages and Routes of Administration

In general, methods disclosed herein comprise administering a therapeutic agent by oral administration. However, in some instances, methods comprise administering a therapeutic agent by intraperitoneal injection. In some instances, methods comprise administering a therapeutic agent in the form of an anal suppository. In some instances, methods comprise administering a therapeutic agent by intravenous (“i.v.”) administration. It is conceivable that one may also administer therapeutic agents disclosed herein by other routes, such as subcutaneous injection, intramuscular injection, intradermal injection, transdermal injection percutaneous administration, intranasal administration, intralymphatic injection, rectal administration intragastric administration, or any other suitable renteral administration. In some embodiments, routes for local delivery closer to site of injury or inflammation are preferred over systemic routes. Routes, dosage, time points, and duration of administrating therapeutics may be adjusted. In some embodiments, administration of therapeutics is prior to, or after, onset of either, or both, acute and chronic symptoms of the disease or condition.

An effective dose and dosage of therapeutics to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition (e.g., reduced instances of diarrhea, rectal bleeding, weight loss, and size or number of intestinal lesions or strictures, reduced fibrosis or fibrogenesis, reduced fibrostenosis, reduced inflammation). In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic agent is not therapeutically effective or is not providing a sufficient alleviation of the disease or condition, or symptom of the disease or condition, then the dosage amount and/or route of administration may be changed, or an additional agent may be administered to the subject, along with the therapeutic agent. In some embodiments, as a patient is started on a regimen of a therapeutic agent, the patient is also weaned off (e.g., stepwise decrease in dose) a second treatment regimen.

Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day. Non-limiting examples of effective dosages of for oral delivery of a therapeutic agent include between about 0.1 mg/kg and about 100 mg/kg of body weight per day, and preferably between about 0.5 mg/kg and about 50 mg/kg of body weight per day. In other instances, the oral delivery dosage of effective amount is about 1 mg/kg and about 10 mg/kg of body weight per day of active material. Non-limiting examples of effective dosages for intravenous administration of the therapeutic agent include at a rate between about 0.01 to 100 μmol/kg body weight/min. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

In some embodiments, the administration of the therapeutic agent is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges may be adjusted based on subject's response to the treatment. Some routes of administration will require higher concentrations of effective amount of therapeutics than other routes.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%~100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

### 5,5 Kits

Further provided is a kit to treat IBD (e.g., CD, UC and/or mrUC). The kit comprises of the antibodies described herein, which can be used to perform the methods described herein. The kit is useful for practicing the inventive method of providing treatment to an IBD, CD, UC and/or mrUC patient by administering an anti-TL1A antibody. The kit is an assemblage of materials or components, including at least one of the inventive compositions. Thus, in some embodiments, the kit contains a composition including anti-TL1A antibodies, for the treatment of IBD, CD, UC and/or MR-UC, as described above. In other embodiments, the kits contain all of the components necessary and/or sufficient to perform a detection assay for TL1A, including all controls, directions for performing assays, and any necessary software for analysis and presentation of results.

The exact nature of the components configured in the inventive kit depends on its intended purpose. For example, some embodiments are configured for the purpose of treating IBD, CD, UC and/or MR-UC. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or alleviate IBD, CD, UC and/or MR-UC. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of an inventive composition containing anti-TL1A antibodies and/or primers and probes for TL1A. The packaging material generally has an external label which indicates the contents and/or purpose of the kit and/or its components.

Disclosed herein, are kits useful for to detect TL1A isoform expression or proxy marker disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of IBD. In other embodiments, the kits contain all of the components necessary and/or sufficient to perform an assay for detecting and measuring IBD markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose.

In some embodiments, the kits described herein are configured for the purpose of treating and/or characterizing a disease or condition (e.g., Crohn's disease), or subclinical phenotype thereof (e.g., stricturing, penetrating, or stricturing and penetrating disease phenotypes) in a subject. In some embodiments, the kits described herein are configured for the purpose of identifying a subject suitable for treatment with an inhibitor of TL1A activity or expression (e.g., anti-TL1A antibody). In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with a therapeutic agent disclosed herein. An exemplary therapeutic agent is an anti-TL1A antibody.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### 5.6 Systems

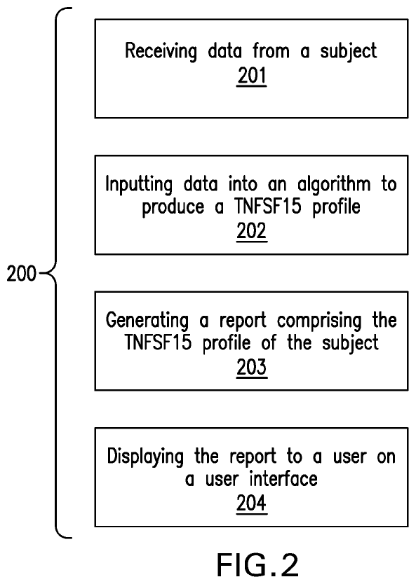

Disclosed herein are systems for treating a subject with an inhibitor of TL1A activity or expression (e.g., anti-TL1A antibody. In some embodiments, the systems described herein comprise kits and compositions for detecting the TL1A isoform expression or proxy marker described herein in a biological sample of a subject. The system may comprise a computer system for implementing one or more methods of the disclosure, such as for example, receiving data of TL1A isoform expression or proxy marker of a subject 201, inputting the data of TL1A isoform expression or proxy marker into an algorithm to produce a TNFSF15 profile 202, and generating a report comprising the TNFSF15 profile of the subject 203, and displaying the report to a user on a graphical user interface 204, as shown in FIG. 2. A “TNFSF15 profile” as used herein refers to a profile of TL1A isoform expression or proxy marker described herein of a subject that is detected in a biological sample obtained from the subject. In some embodiments, a TNFSF15 profile comprises a positive, a negative, or an indeterminate result (e.g., therapeutic response to treatment with an inhibitor of TL1A activity or expression).

### 5.6.1 Computer Systems

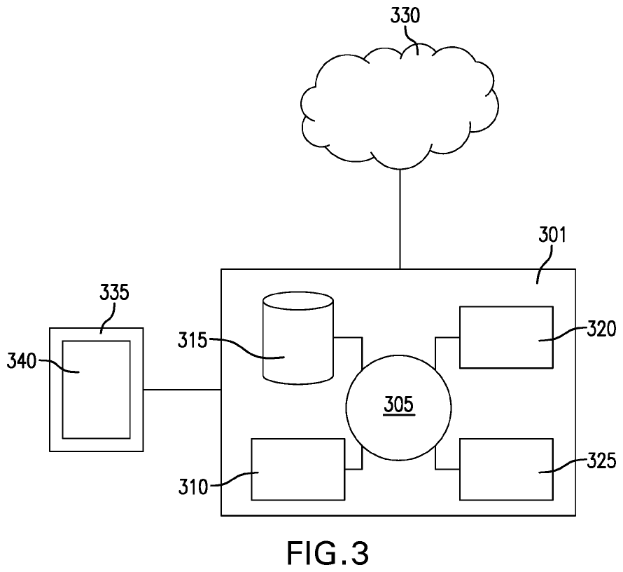

FIG. 3 shows a computer system 301 that is programmed or otherwise configured to generate a TNFSF15 profile for a subject in need thereof. The computer system 301 can regulate various aspects of producing the TNFSF15 profile (e.g., receiving data of TL1A isoform expression or proxy marker, generating a report with the TNFSF15 profile of the biological sample, and displaying the report to a user), of the present disclosure, such as, for example, by including permissions or encryption of data of TL1A isoform expression or proxy marker and/or TNFSF15 profile of the subject to ensure patient privacy.

The computer system 301 can be an electronic device of a user or a computer system that is remotely located with respect to the electronic device. The electronic device can be a mobile electronic device, such as a mobile electronic device belonging to a physician.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. In some embodiments, the software comprises the Assay Interpretation Software, as disclosed elsewhere herein. In some embodiments, the software comprises the Model Identification Software as disclosed elsewhere herein. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a report comprising the TNFSF15 profile of the subject or other relevant clinical information for purposes of informing a selection of a therapeutic agent (e.g., anti-TL1A antibody) to treat a disease or condition of the subject described herein. Examples of UI's include, without limitation, a graphical user interface (GUI) and web-based user interface. In some embodiments, the computer system comprises one or more input devices. Non-limiting examples of input devices comprise a computer mouse, keyboard, microphone, trackball, scanner, joystick, camera, or any other suitable input device.

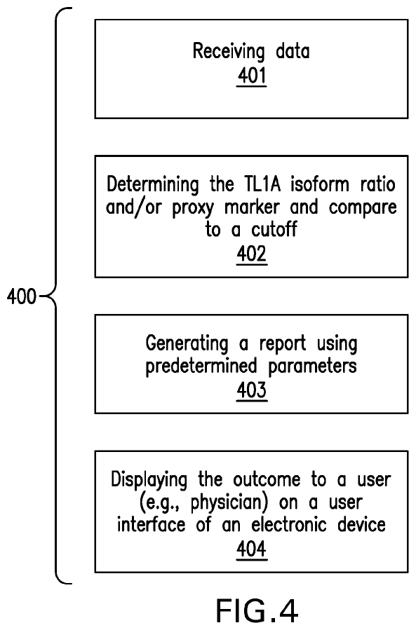

Methods and systems of the present disclosure can be implemented by way of one or more algorithms. An algorithm can be implemented by way of software upon execution by the central processing unit 305. The algorithm can, for example, perform: (a) receiving data of TL1A isoform expression or proxy marker of a subject 401. (b) determining TL1A isoform ratio 402. (c) generating an outcome using predetermined parameters 403, and (d) displaying the outcome to a user (e.g., physician) on a user interface of an electronic device 404, as shown in FIG. 4. In some embodiments, the outcome is positive, negative or indeterminant. In some embodiments, the predetermined parameters are based on cutoff predictive of a therapeutic response to a treatment, such as with an inhibitor of TL1A activity or expression.

### 5.6.2 Web Application

In some embodiments, the computer system comprises software for a web application. In light of the disclosure provided herein, those of skill in the art will recognize that a web application may utilize one or more software frameworks and one or more database systems. A web application, for example, is created upon a software framework such as Microsoft® NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash R®, HTML 5, Apple® QuickTime®, Microsoft® Silverlight®, Java™, and Unity®.

### 5.6.3 Mobile Application

In some embodiments, the computer system comprises software for a mobile application. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, R, SQL, MATLAB, Scala, Julia, SAS, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

### 5.6.4 Standalone Application

In some embodiments, the computer system comprises software a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™. Lisp. Python™, Visual Basic, and VB.NET, or combinations thereof. Compilation may be often performed, at least in part, to create an executable program. In some instances, a computer program includes one or more executable complied applications.

### 5.6.5 Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### 5.6.5.1 Model Identification Software

Disclosed herein is model identification software that employs a machine learning approach to identify models employing data of TL1A isoform expression or proxy marker that can be used to predict a positive therapeutic response to an inhibitor of TL1A activity or expression.

In some embodiments, the model identification software comprises one or more algorithms disclosed herein. In some embodiments the one or more algorithms comprises a classifier configured to classify samples by assigning output values. In some embodiments, the output values comprise descriptive labels, numerical values, or a combination thereof. In some embodiments, the algorithm is a linear classifier. In some embodiments, the linear classifier algorithm is a logistic regression. In some embodiments, the linear classifier algorithm is Naive Bayes classifier. In some embodiments, the linear classifier algorithm is Fisher's linear discriminant. In some embodiments, the algorithm is support vector machine. In some embodiments, the support vector machine algorithm is least squares support vector machines. In some embodiments, the algorithm is quadratic classifier. In some embodiments, the algorithm is Kernel estimation. In some embodiments, the Kernel estimation algorithm is k-nearest neighbor. In some embodiments, the algorithm is a decision tree. In some embodiments, the decision tree algorithm is random forest. In some embodiments, the algorithm is neural network. In some embodiments, the algorithm is learning vector quantization.

Some of the output values may comprise descriptive labels. Such descriptive labels may provide an identification or indication of the positive output or a negative output. In some embodiments, the positive output comprises high expression of a protein or RNA (e.g., TL1A). In some embodiments, the negative output comprises low expression of a protein or RNA (e.g., TL1A). In some embodiments, the output values comprise a relative output, such as a high-risk, an intermediate-risk, or a low-risk, which is relative to an index or a control. In some embodiments, the index or the control is derived from reference subjects that have responded to a treatment with an inhibitor of TL1A activity or expression (positive control), and/or reference subjects that have not responded to a treatment with an inhibitor of TL1A activity or expression (negative control).

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has a TL1A isoform ratio above a cutoff. As another example, a binary classification of samples may assign an output value of “negative” or 0 if the sample indicates that the subject has a TL1A isoform ratio below a cutoff. In this case, a single cutoff value for the TL1A isoform ratio is used to classify samples into one of the two possible binary output values or classes of individuals.

In another example, a binary classification of samples may assign an output value of “positive” or 1 if the sample indicates that the subject is a high producer of gene expression (e.g., TL1A). As another example, a binary classification of samples may assign an output value of “negative” or 0 if the sample indicates that the subject is a low producer of gene expression. In some embodiments, a high producer is relative to a cutoff value of mean value, median value, slope of TL1A expression time curve of at least 10 degree, slope of TL1A expression time curve of at least 10 degree, slope of TL1A expression time curve of at least 15 degree, slope of TL1A expression time curve of at least 20 degree, slope of TL1A expression time curve of at least 25 degree, slope of TL1A expression time curve of at least 30 degree, slope of TL1A expression time curve of at least 35 degree, slope of TL1A expression time curve of at least 40 degree, slope of TL1A expression time curve of at least 10 degree, slope of TL1A expression time curve of at least 45 degree, or slope of TL1A expression time curve of at least 50 degree. In some embodiments, a low producer is relative to a cutoff value of mean value, median value, or slope of TL1A expression time curve of 10 degree. In some embodiments the cutoff value

The classifier may be configured to classify samples by assigning output values based on one or more cutoff values. For example, a binary classification of samples may assign an output value of “positive” or 1 if the sample indicates that the subject at least a 50% probability of responding to an inhibitor of TL1A activity or expression, thereby assigning the subject to a class of individuals receiving a positive result. As another example, a binary classification of samples may assign an output value of “negative” or 0 if the sample indicates that the subject has less than 50% probability of responding to an inhibitor of TL1A activity or expression, thereby assigning the subject to a class of individuals receiving a negative test result. In this case, a single cutoff value of 50% is used to classify samples into one of the two possible binary output values or classes of individuals. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of responding to an inhibitor of TL1A activity or expression of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of responding to an inhibitor of TL1A activity or expression of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of responding to an inhibitor of TL1A activity or expression of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of responding to an inhibitor of TL1A activity or expression of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of responding to an inhibitor of TL1A activity or expression. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

In some embodiments, the TL1A isoform ratio and/or proxy marker are predictive of a positive therapeutic response to an inhibitor of TL1A activity or expression with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of predicting a positive therapeutic response to an inhibitor of TL1A activity or expression using the algorithm may be calculated as the percentage of samples identified or classified as responding to an inhibitor of TL1A activity or expression that correspond to subjects that truly responded to an inhibitor of TL1A activity or expression.

In some embodiments, the TL1A isoform ratio and/or proxy marker are predictive of a positive therapeutic response to an inhibitor of TL1A activity or expression with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of predicting a positive therapeutic response to an inhibitor of TL1A activity or expression using the algorithm may be calculated as the percentage of samples identified or classified as having a positive therapeutic response to an inhibitor of TL1A activity or expression that correspond to subjects that truly responded to an inhibitor of TL1A activity or expression.

In some embodiments, the TL1A isoform ratio and/or proxy marker are predictive of a positive therapeutic response to an inhibitor of TL1A activity or expression with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of predicting a positive therapeutic response to an inhibitor of TL1A activity or expression using the algorithm may be calculated as the percentage of independent test samples associated with a positive therapeutic response to an inhibitor of TL1A activity or expression (e.g., subjects known to have responded to the inhibitor of TL1A activity or expression) that are correctly identified or classified as having a positive therapeutic response to an inhibitor of TL1A activity or expression.

In some embodiments, the TL1A isoform ratio and/or proxy marker are predictive of a positive therapeutic response to an inhibitor of TL1A activity or expression with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of predicting a positive therapeutic response to an inhibitor of TL1A activity or expression using the algorithm may be calculated as the percentage of independent test samples associated with absence of a positive therapeutic response to an inhibitor of TL1A activity or expression (e.g., subjects with negative clinical test results for response to an inhibitor of TL1A activity or expression) that are correctly identified or classified as not having a positive therapeutic response to an inhibitor of TL1A activity or expression.

In some embodiments, the TL1A isoform ratio and/or proxy marker identify the presence (e.g., positive test result) or absence (e.g., negative test result) of a positive therapeutic response to an inhibitor of TL1A activity or expression with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the algorithm in classifying samples as having or not having a positive therapeutic response to an inhibitor of TL1A activity or expression. The AUC may range from a value of 0 to 1, where an AUC of 0.5 is indicative of a completely random classifier (e.g., a coin flip) and an AUC of 1 is indicative of a perfectly accurate classifier (with sensitivity of 100% and specificity of 100%).

The one or more algorithms in the model identification software may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the positive therapeutic response to an inhibitor of TL1A activity or expression. The classifiers may be adjusted or tuned by adjusting parameters of the classifiers (e.g., a set of cutoff values used to classify a sample as described elsewhere herein) to improve or optimize the performance metrics. The one or more classifiers may be adjusted or tuned so as to reduce an overall classification error.

### 5.6.5.2 Assay Interpretation Software

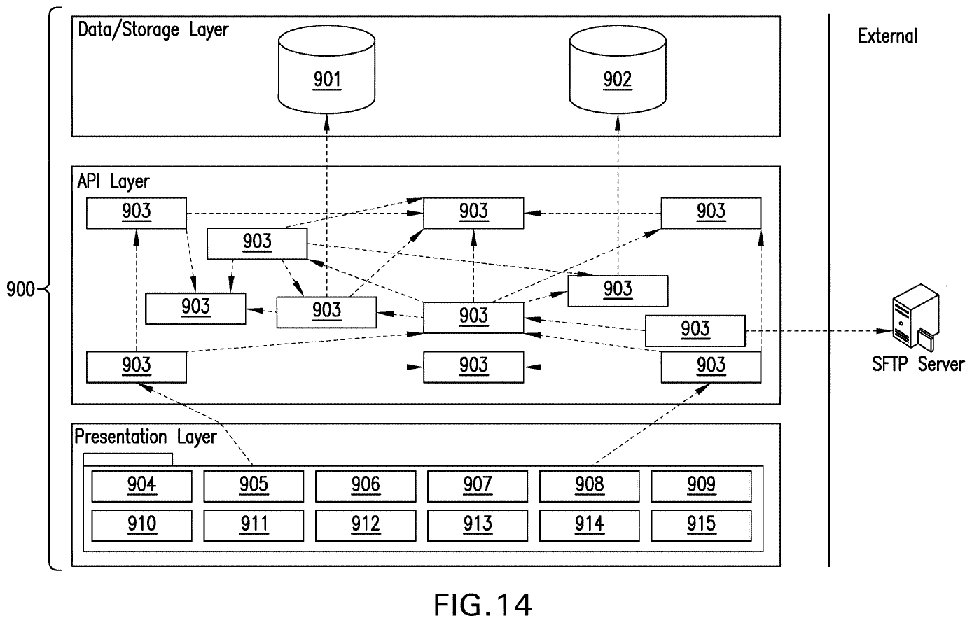

In some embodiments, the software modules described herein comprise data (e.g. data of the TL1A isoform ratio and/or proxy marker) analysis software modules or display software modules, or combinations thereof as shown in FIG. 14. In some embodiments, the data (e.g. data of the TL1A isoform ratio and/or proxy marker) analysis software modules are communicatively coupled to each other. In some embodiments, the data (e.g. data of the TL1A isoform ratio and/or proxy marker) analysis software modules are configured to receive data, analyze data, and/or transmit data to other data analysis software modules within the API laver, or to one or more databases 901, 902 in the data/storage layer. In some embodiments, the data (e.g. data of the TL1A isoform ratio and/or proxy marker) analysis software modules 903 are communicatively coupled to one or more databases described herein. Display software modules disclosed herein within the display layer enable the display of a result from the data analysis performed by the data analysis software modules. In some embodiments, the display software modules are implemented as a web application, a mobile application, or a standalone application.

The data (e.g. data of the TL1A isoform ratio and/or proxy marker) analysis software modules instruct the one or more computer processors to output a result for a sample. In some embodiments, the result is positive, which indicates that the subject from which the sample was obtained is predicted to respond to an inhibitor of TL1A activity or expression (e.g., an anti-TL1A antibody or antigen binding fragment thereof). In some embodiments, the result is negative, which indicates that the subject from which the sample was obtained is not predicted to respond to the inhibitor of TL1A activity or expression. In some embodiments, the result is indeterminate.

In some embodiments, the data (e.g. data of the TL1A isoform ratio and/or proxy marker) analysis software module comprises one or more algorithms configured to instruct one or more processers to perform the methods disclosed herein (e.g., analyze data (e.g. data of the TL1A isoform ratio and/or proxy marker) to produce a TL1A profile). In some embodiments, the one or more algorithms comprises a Brute Force algorithm. In some embodiments, the one or more algorithms comprises a Greedy algorithm. In some embodiments, the one or more algorithms comprises a Recursive algorithm. In some embodiments, the one or more algorithms comprises a Backtracking algorithm. In some embodiments, the one or more algorithms comprises a Divide & Conquer algorithm, In some embodiments, the one or more algorithms comprises a Dynamic programming algorithm. In some embodiments, the one or more algorithms comprises a Randomized algorithm. In some embodiments, the data (e.g. data of the TL1A isoform ratio and/or proxy marker) analysis software module comprises a script of a plurality of such algorithms that is configured to analyze data in a data file disclosed herein.

In some embodiments, the algorithm is configured to determine the TL1A isoform ratio or proxy marker disclosed herein. In some embodiments, the algorithm may analyze the data of the TL1A isoform ratio and/or proxy marker, and compares the TL1A isoform ratio or the proxy marker determined in the sample with a cutoff or reference that has been determined to be associated with a positive result as described elsewhere herein. If the algorithm determines the presence of a level of the TL1A isoform ratio and/or proxy marker associated with a positive result, then a positive result is assigned to the sample. If the algorithm determines the absence of a level of the TL1A isoform ratio and/or proxy marker associated with a positive result, then a negative result is assigned to the sample.

In some embodiments of the methods provided herein, including in Sections 2, 5 (including in the paragraphs of Section 5.6.5), and 7, determining a positive result comprises (1) determining a positive result when the proxy marker is above a cutoff if the proxy marker has a positive correlation with TL1A isoform ratio; or (2) determining a positive result when the proxy marker is below a cutoff if the proxy marker has a negative correlation with TL1A isoform ratio. In some embodiments of the methods provided herein, including in Sections 2, 5 (including in the paragraphs of Section 5.6.5), and 7, determining a positive result comprises determining a positive result when the TL1A isoform ratio is above a cutoff, wherein the TL1A isoform ratio is calculated from expression level of TL1A long and short isoforms determined from a sample from the subject.

In some embodiments, when the proxy marker is one or more SNP, each genotype of a SNP is assigned a numerical value and weights are applied to those values based on the significance of the SNP in predicting a positive therapeutic response to the inhibitor of TL1A activity or expression. For example, a genotype may be assigned the number “0” for two copies of the minor allele, a “1” for one copy of the major allele and one copy of the minor allele, or a “2” for two copies of the major allele. In some embodiments, the algorithm calculates the sum of the numerical values it assigns for each SNP in the model. In some embodiments, the algorithm weights each SNP based on its relative significance in predicting a positive therapeutic response to the inhibitor of TL1A activity or expression, by giving SNPs with less significance a smaller weight than those SNPs having a larger significance. For example a particular SNP may be given a weight of 1.5, meaning that the possible score could be 0, 1.5, or 3, if its significance in predicting positive therapeutic response to the inhibitor of TL1A activity or expression is higher than the others in the model. In some embodiments, the weight comprises multiplier of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0 or 5.0.

In some embodiments of the various methods and systems provided herein including in Sections 2, 5 (including but not limited to Sections 5.6.1, 5.6.5, and 5.6, 11) and 7, the TL1A profile comprises or consists of the TL1A isoform ratio and/or proxy marker. In some embodiments of the various methods and systems provided herein including in Sections 2, 5 (including but not limited to Sections 5.6.1, 5.6.5, and 5.6.11) and 7, the TL1A profile comprises or consists of TL1A isoform ratio. In some embodiments of the various methods and systems provided herein including in Sections 2, 5 (including but not limited to Sections 5.6.1, 5.6.5, and 5.6.11) and 7, the TL1A profile comprises or consists of a proxy marker.

### 5.6.5.3 Assay Interpretation Software Architecture

The software architecture described herein may be a layered architecture pattern, wherein the individual layers (e.g., horizontal layers) may be interconnected. In some embodiments, the software architecture is provided in FIG. 14, which illustrates three horizontal layers in the architecture. In some embodiments, the layered architecture pattern comprises 2, 3, 4, 5, 6, 7, 8, 9, or 10 layers. In some embodiments, the layered architecture pattern comprises between 2 and 10, 3 and 9, 4 and 9, or 5 and 7 layers. In some embodiments, the layered architecture pattern is a hybrid layered architecture, wherein additional layers are added or existing layers are modified. In some embodiments, one or more of the layers is optional (or “open”). In some embodiments, one or more of the layers is required (or “closed”). In some embodiments, all of the layers in the layered architecture pattern are closed. In some embodiments, each layer comprises similar software modules or “components,” such that each layer is relatively isolated from the others to perform a set of related tasks. In some embodiments, each component is interconnected to at least one other component within the same layer, and optionally connected with components in different layers. In some embodiments, the software architecture is a central computing environment, in which a majority of the computing is performed by a central processor. In some embodiments, the software architecture is a Distributed Computing Environment (DCE).

Referring to FIG. 14, the layered architecture pattern may comprise a data/storage layer, an API layer, or a presentation layer, or any combination thereof. In some embodiments, the data/storage layer comprises one or more databases 901, 902. The one or more databases is configured to store data, including by not limited to the data of the TL1A isoform ratio and/or proxy marker, one or more TL1A profile(s) of a subject, clinical study samples, algorithms, metadata, and the like). In some embodiments, the API layer comprises data (e.g. data of the TL1A isoform ratio and/or proxy marker) analysis software modules 903. In some embodiments, the data (e.g. data of the TL1A isoform ratio and/or proxy marker) analysis software modules 903 are custom rules or algorithms that handle the exchange of information between the one or more databases and a user interface disclosed herein. In some embodiments, the data (e.g. data of the TL1A isoform ratio and/or proxy marker) analysis software modules 903 instruct the one or more processors of the computer system to analyze the data disclosed herein and generate a TL1A profile for a subject. In some embodiments, the presentation layer comprises display software modules 904-915. In some embodiments, the presentation layer comprises components related to the presentation of information related to the data (e.g. data of the TL1A isoform ratio and/or proxy marker) analysis to a user (e.g., data (input), the TL1A profile (output) for a sample). In some embodiments, the display software modules comprises a login view 904, a files view 905, a samples view 906, a data analyses view 907, a new data analysis view 908, an algorithm editor view 909, a system settings view 910, a clinical settings view 911, a clinical study settings view 912, a user management view 913, an activities view 914, or a my profile view 915, or any combination thereof.

In some embodiments, the data (e.g. data of the TL1A isoform ratio and/or proxy marker) analysis software modules 903 are communicatively coupled to each other. In some embodiments, the data (e.g. data of the TL1A isoform ratio and/or proxy marker) analysis software modules 903 are configured to receive data, analyze data, and/or transmit data to other data analysis software modules 903 within the API layer, or to one or more databases 901, 902 in the data/storage layer. In some embodiments, the data (e.g. data of the TL1A isoform ratio and/or proxy marker) analysis software modules 903 are communicatively coupled to one or more databases 901, 902 described herein. Display software modules 904-915 disclosed herein within the display layer enable the display of a result from the data (e.g. data of the TL1A isoform ratio and/or proxy marker) analysis performed by the data analysis software modules 903. In some embodiments, the display software modules are implemented as a web application, a mobile application, or a standalone application.

In some embodiments, the layered architecture pattern comprises one or more additional layers comprising a service layer, or a persistence layer, or any combination thereof.

### 5.6.6 Databases

The medium, method, and system disclosed herein comprise one or more databases, or use of the same. In view of the disclosure provided herein, those of skill in the art will recognize that many databases are suitable for storage and retrieval of data (e.g. data of the TL1A isoform ratio and/or proxy marker), a TL1A profile of a subject, clinical study data user preference and login information, and the like. Suitable databases include, by way of non-limiting examples, relational databases, non-relational databases, object oriented databases, feature databases, entity-relationship model databases, associative databases, XML databases, document oriented databases, and graph databases. Further non-limiting examples include SQL, PostgreSQL, MySQL, Oracle, DB2, Sybase, and MongoDB.

Referring to Assay Interpretation Software architecture provide in FIG. 14, in some embodiments, the computer systems described herein comprise one or more databases 901, 902. In some embodiments, the software modules 903 are communicatively coupled to the one or more databases 901, 902. In some embodiments, the one or more database 901, 902 is internet-based. In some embodiments, the one or more databases 901, 902 is web-based. In some embodiments, the one or more databases 901, 902 is cloud computing-based. In some embodiments, the one or more databases 901, 902 may be based on one or more local computer storage devices.

### 5.6.7 Data Transmission

The subject matter described herein, including methods for producing a TNFSF15 profile are configured to be performed in one or more facilities at one or more locations. Facility locations are not limited by country and include any country or territory. In some instances, one or more steps are performed in a different country than another step of the method. For example, referring to FIG. 2, the step of receiving data (e.g. data of the TL1A isoform ratio and/or proxy marker) from a subject 201, inputting the data into the algorithm to produce a TL1A profile 202, and generating a repot comprising the TL1A profile of the subject 203 may be performed in a different country or territory than displaying the report to the user on the user interface 204.

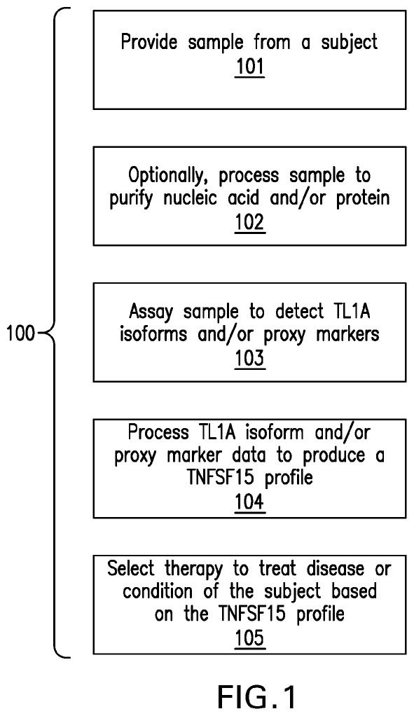

In some instances, one or more steps for obtaining a biological sample are performed in a different country than one or more steps for analyzing the biological sample, such as detecting the presence or absence of a TL1A isoform ratio and/or proxy marker in the biological sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein (e.g. data of the TL1A isoform ratio and/or proxy marker). For example, referring to FIG. 1, the step of providing a buccal swab sample from the subject 101 may be performed at a facility or location that is separate from the sample processing facility where the step of assaying the sample to determine the TL1A isoform ratio or proxy marker 103 is performed. In some embodiments, the step of processing the data (e.g. data of the TL1A isoform ratio and/or proxy marker) to produce the TL1A profile 104 is performed at a data processing facility that may be separate from the sample processing facility. In some embodiments of the methods and systems described herein, the analysis is performed and a subsequent data transmission step will convey or transmit the results of the analysis.

As shown in FIG. 14, in some embodiments, the results of the analysis are transmitted from one or more software modules 903 in the API layer to one or more software modules in the presentation layer, which enable a user to view the results in one or more views described herein. In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy (e.g., anti-TL1A therapy), or the like, in the same or different location or country.

### 5.6.8 User

The users of the computer systems disclosed herein may have different privileges when using the software programs disclosed herein. In some embodiments, the user comprises a super user, an administrator, a researcher, a clinical technician, or a technology, as shown in FIG. 151. In some embodiments, the design of the software is such that certain software modules (data analysis software modules, display software modules) are inaccessible to certain users.

### 5.6.9 User Interface

The computer systems disclosed herein, in some embodiments, comprise a user interface for displaying information related to an analysis, such as a result to a user. Referring to FIG. 14, one or more display software modules 904-915 in the display layer instructs the one or more computer processors to display information related to an analysis of a sample to the user. In some embodiments, the information related to ana analysis of a sample comprises the data (uploaded), samples analyzed, algorithm(s) used, clinical settings, or results from the analyses performed. In some embodiments, the one or more display software modules runs as a web application, a standalone application, or a mobile application as disclosed herein. In some embodiments, the one or more display software modules in the display layer comprises a login view 904, a files view 905, a samples view 906, a data analyses view 907, a new data analysis view 908, an algorithm editor view 909, a system settings view 910, a clinical settings view 911, a clinical study settings view 912, a user management view 913, an activities view 914, or a my profile view 915, or any combination thereof.

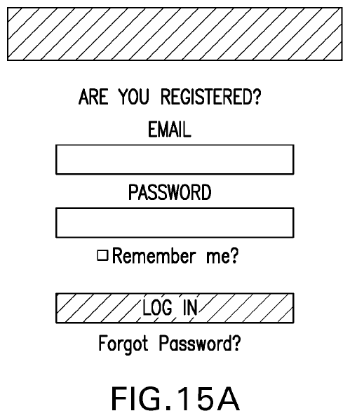

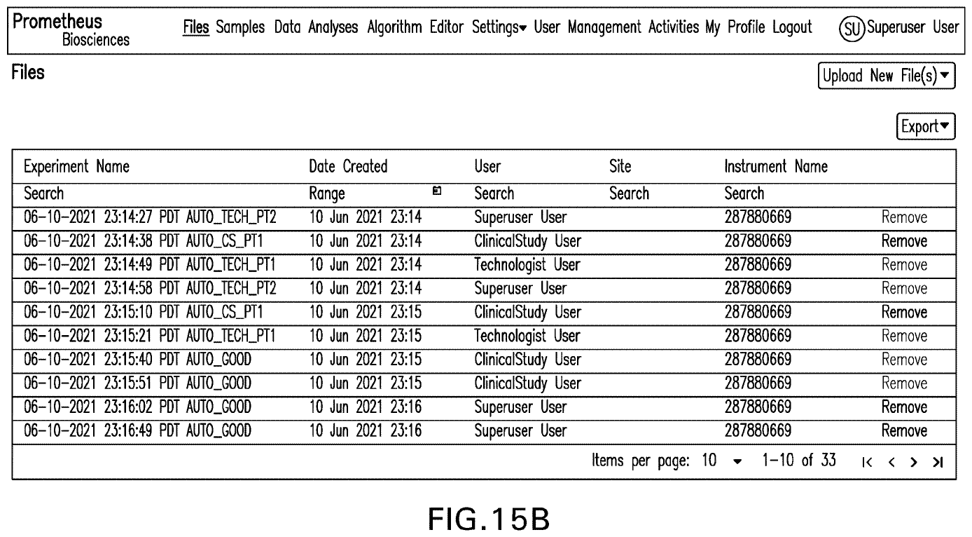

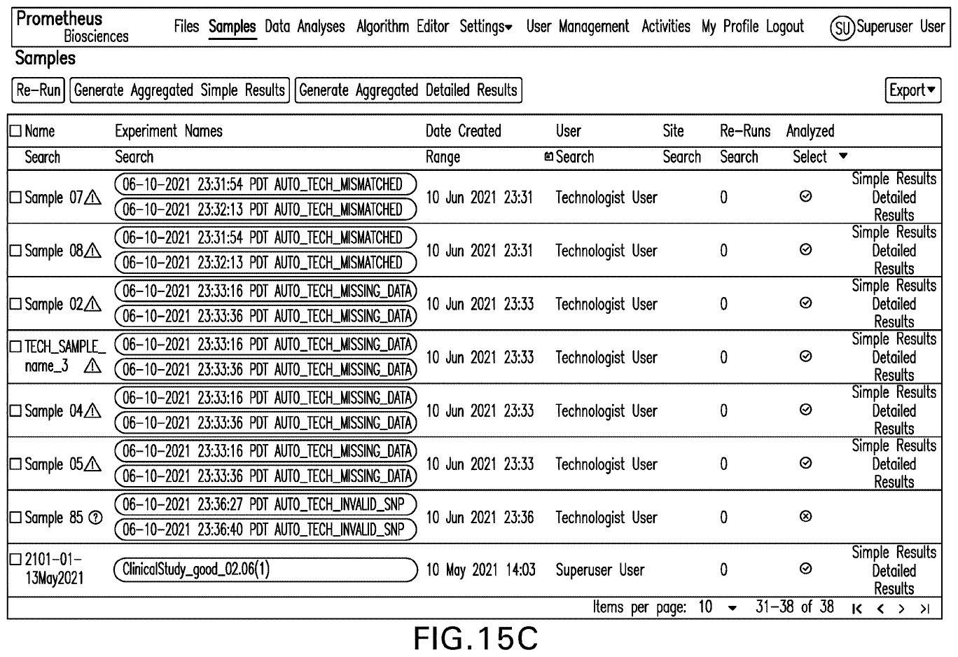

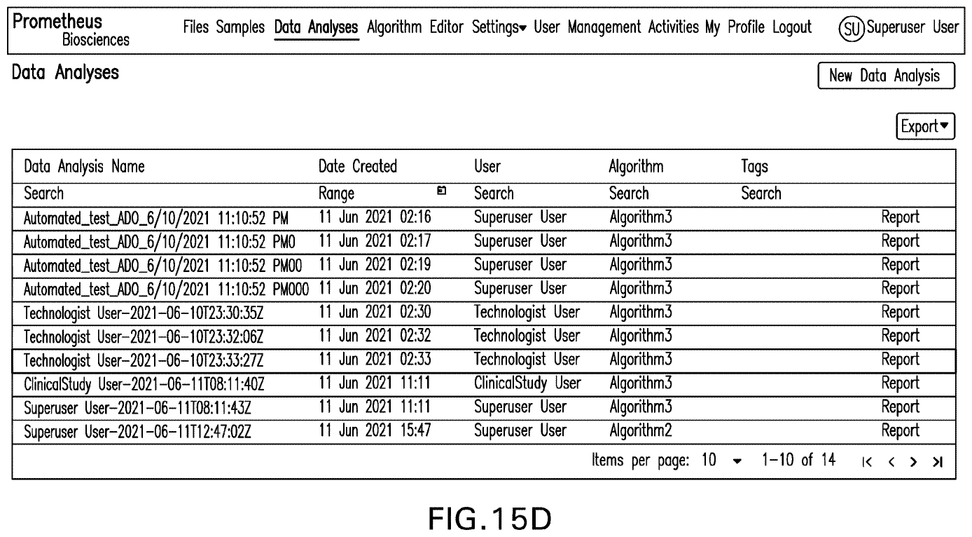

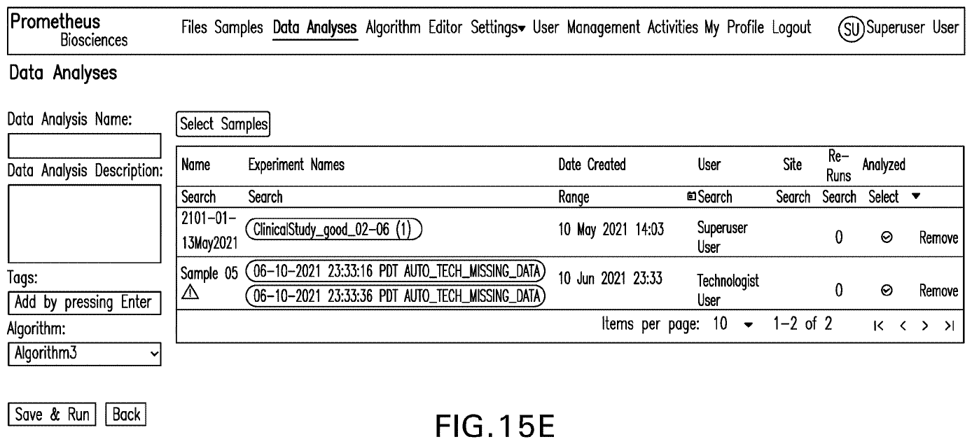

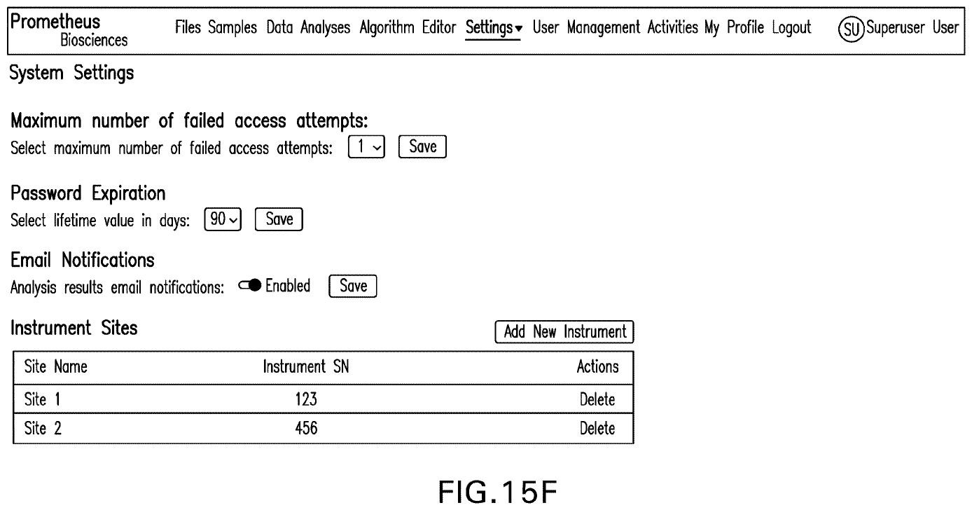

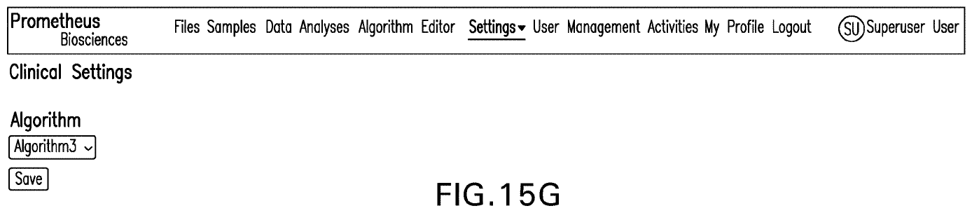

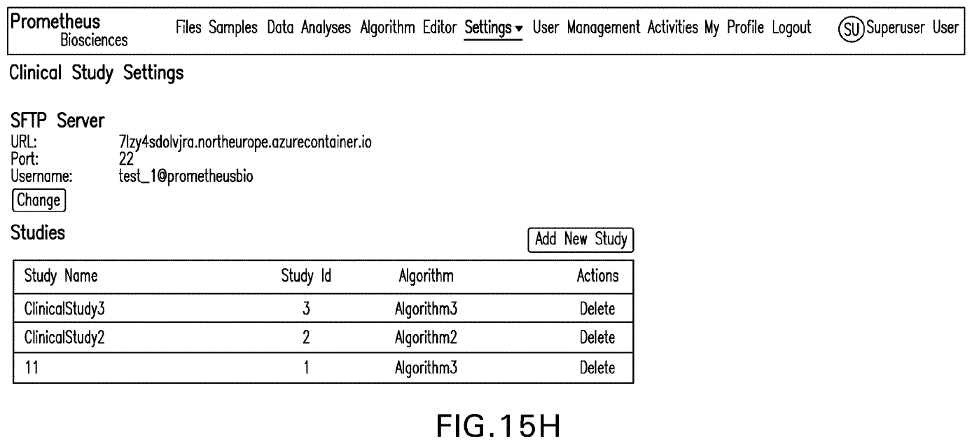

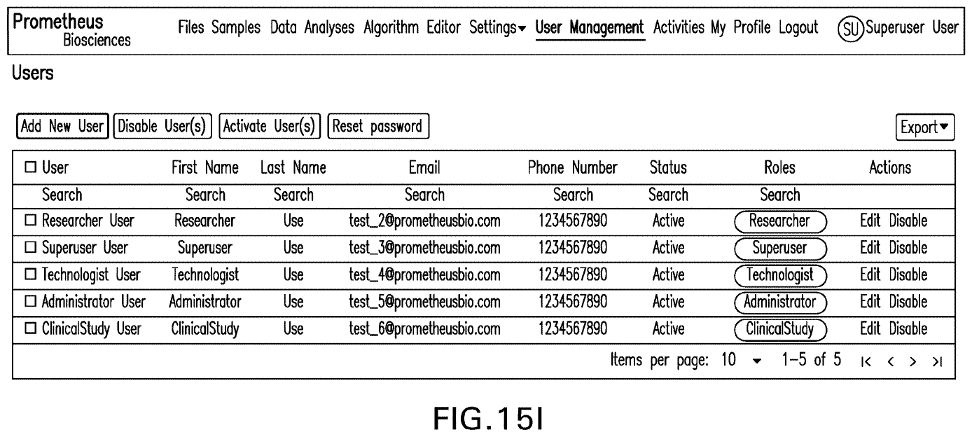

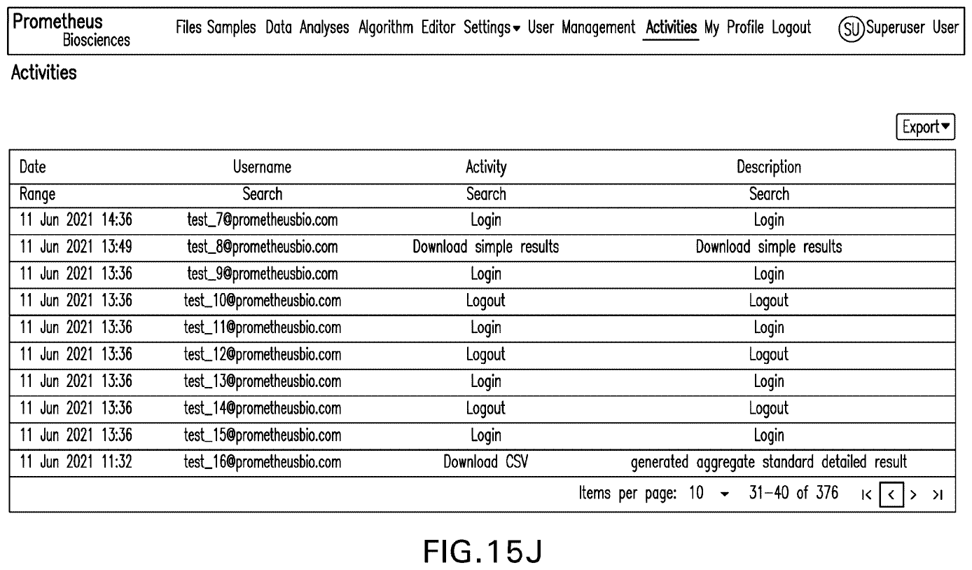

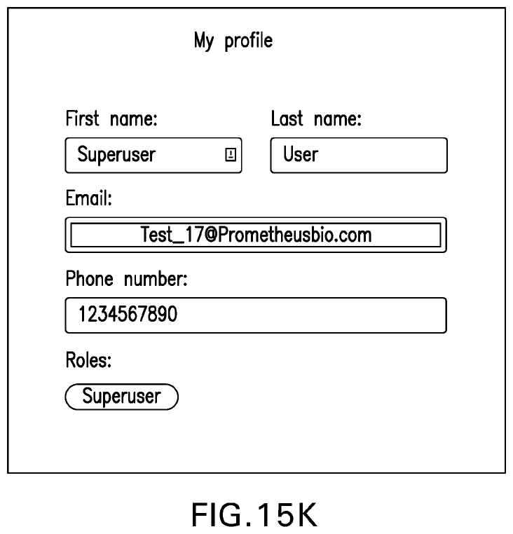

In some embodiments, the login view 904 is provided in FIG. 15A. In some embodiments, the user logs in as a registered user to a portal and uploads a data file comprising the data of the TL1A isoform ratio and/or proxy marker for analysis by the data analysis software modules 903. In some embodiments when the proxy marker comprises a SNP, the data for the genotype of the SNP can be numerical, where a “1” is assigned for one copy of the major allele and one copy of the minor allele, a “0” is assigned for two copies of the minor allele, and “2” is assigned for two copies of the major allele. In some embodiments, the data or the data file further comprises (i) the patient identification (ID), (ii) disease type, (iii) collection site ID, (iv) physician ID, (v) both the patient ID and disease type. (vi) both the patient ID and collection site ID, (vii) both the patient ID and physician ID, (viii) both disease type and collection side ID, (ix) both disease type and physician ID, (x) both collection side ID and physician ID, (xi) the patient ID, disease type and collection side ID, (xii) the patient ID, disease type, and physician ID, (xiii) the patient ID, collection side ID, and physician ID, (xiv) disease type, collection side ID, and physician ID, or (xv) the patient ID, disease type, collection side ID, and physician ID.

In some embodiments, the samples view 906 is provided in FIG. 15C, in the samples view 906, the user may rerun the analysis of the samples, generate a report to display the results, or export the table content. In some embodiments, the sample view provides to the user one or more of the name of the sample, the experiment name, the date created, the user (e.g., the user that uploaded the first sample information), the site (site location of the instrument that created the TL1A isoform or proxy marker data file), reruns (number of reruns performed for the selected sample), analyzed (true/false if the sample was analyzed), or a simple result from the data analysis. In some embodiments, the data (e.g. data of the TL1A isoform ratio and/or proxy marker) file was created by a polymerase chain reaction (PCR) instrument, such as a real-time PCT instrument. In some embodiments, the data file is a FASTQ file created from a next generation sequencing instrument.

In some embodiments, the data analyses view 907 is provided in FIG. 15D. The data analysis view 907 may only be viewed by certain uses, such as those with additional privileges (e.g., super user and researcher). In this view, the user can create a new analysis with custom configurations, search and filter the results, select a different algorithm for a rerun of the samples, and export a version of the report that contain additional details. In some embodiments, the new data analysis view 908 is provided in FIG. 15E. The new data analysis view 908 is used by super users or researchers to configure and run custom analyses.

In some embodiments, the algorithm editor view 909 allows superusers and researchers to create a new algorithm from scratch, or modify an existing algorithm, such as to optimize the TL1A isoform and proxy marker to detect in a sample and the corresponding cutoff to provide a positive or a negative result.

In some embodiments, the system settings view 910 is provided in FIG. 15F. The system settings view 910 allows superusers and administrators to change the configurations of the account settings (e.g., failed login attempts, password lifetime expiration).

In some embodiments, the clinical settings view 911 is provided in FIG. 15G. The clinical settings view 911 permits superusers to change the default algorithms for certain samples. In some embodiments, the clinical study settings view 912 is provided in FIG. 15H. The clinical study setting view permits superusers to change the default algorithms used for certain samples depending on the study they came from.

In some embodiments, the user management view 913 is provided in FIG. 151. The user management view 913 permits superusers and administrators to manage the account by adding new users to the platform, activate or disable certain users, and the like. In some embodiments, the activities view 914 is provided in FIG. 15J. The activities view 914 allows any user to trace the activities of other uses, as well as sort, filter, and/or export the activities in a viewable format. In some embodiments, a my profile view 915 is provided in FIG. 15K. The my profile view 915 can be accessed by each user in the system.

### 5.6.10 Reports

In some embodiments, the results from the analyses described herein (to produce a TL1A profile) are provided to a user in a report. In some embodiments, the reports comprise the result (e.g., positive or negative) for a sample of a subject. In some embodiments, a positive result indicates that the subject is predicted to have a positive therapeutic response to an inhibitor of TL1A activity or expression. In some embodiments, a negative result indicates that the subject is not predicted to have a positive therapeutic response to the inhibitor of TL1A activity or expression. In some embodiments, the report further comprises the date created, the user (e.g., the user that uploaded the first sample information), the site (site location of the instrument that created the TL1A isoform expression or proxy marker data file), reruns (number of reruns performed for the selected sample), or analyzed (true/false if the sample was analyzed), or any combination thereof.

In some embodiments of the methods provided herein, including in Sections 2, 5 (including in the paragraphs of Section 5.6.10), and 7, determining a positive result comprises (1) determining a positive result when the proxy marker is above a cutoff if the proxy marker has a positive correlation with the TL1A isoform ratio; or (2) determining a positive result when the proxy marker is below a cutoff if the proxy marker has a negative correlation with the TL1A isoform ratio. In some embodiments of the methods provided herein, including in Sections 2, S (including in the paragraphs of Section 5.6.10), and 7, determining a positive result comprises determining a positive result when the TL1A isoform ratio is above a cutoff, wherein the TL1A isoform ratio is calculated from the expression levels of TL1A long and short isoform determined from a sample from the subject.

### 5.6.11 Computer-Implemented Methods

The methods described herein may utilize one or more computers. The computer may be used for managing customer and biological sample information such as sample or customer tracking, database management, analyzing molecular profiling data, analyzing cytological data, storing data, billing, marketing, reporting results, storing results, or a combination thereof. The computer may include a monitor or other user interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider (e.g., physician) or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a report with the TNFSF15 profile may enter biological sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### 5.7 Sample Preparation and Assays

The term “express” or “expression” when used in connection with biomarker (e.g. TL1A) refers to the process by which the information carried by the gene of the biomarker becomes manifest as the phenotype, including transcription of the gene to a messenger RNA (mRNA, mRNA expression or gene expression), the subsequent translation of the mRNA molecule to a polypeptide chain and its assembly into the ultimate protein (protein expression).

The term “expression level” of a biomarker refers to the amount or accumulation of the expression product of a biomarker, such as, for example, the amount of a RNA product of the biomarker (the RNA level of the biomarker) or the amount of a protein product of the biomarker (the protein level of the biomarker).

mRNA may be isolated from the starting tissue sample. General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

In some embodiments, the first step in gene expression profiling or gene expression level determination by PCR is the reverse transcription of the RNA template into cDNA, followed by its exponential amplification in a PCR reaction. In other embodiments, a combined reverse-transcription-polymerase chain reaction (RT-PCR) reaction may be used, e.g., as described in U.S. Pat. Nos. 5,310,652; 5,322,770; 5,561,058; 5,641,864; and 5,693,517. The two commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif, USA), following the manufacturer's instructions. The derived cDNA can then be used as a template in the subsequent PCR reaction.

In some embodiments, Real-Time Reverse Transcription-PCR (qRT-PCR) can be used for both the detection and quantification of RNA targets (Bustin, et al., 2005, Clin. Sci., 109:365-379). Examples of qRT-PCR-based methods can be found, for example, in U.S. Pat. No. 7,101,663, which is incorporated by reference herein in its entirety. Instruments for real-time PCR, such as the Applied Biosystems 7500, are available commercially, as are the reagents, such as TaqMan Sequence Detection chemistry.

For example, TaqMan® Gene Expression Assays can be used, following the manufacturer's instructions. These kits are pre-formulated gene expression assays for rapid, reliable detection and quantification of human, mouse and rat mRNA transcripts. TaqMan® or 5′-nuclease assay, as described in U.S. Pat. Nos. 5,210,015; 5,487,972; and 5,804,375; and Holland et al., 1988, Proc. Natl. Acad. Sci. USA 88:7276-7280, can be used. TAQMAN® PCR typically utilizes the 5′-nuclease activity of Taq or Tth polymerase to hydrolyze a hybridization probe bound to its target amplicon, but any enzyme with equivalent S′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

When preparing protein samples from mammalian tissues, some gentle means of enzymatic and/or mechanical disruption can be employed to help separate cells from the more complex tissue matrix. Enzymes used for cell separation include collagenase for breaking down the extracellular matrix and DANase and/or RNAase for breaking down DNA and/or RNA. In certain embodiments, protease inhibitors, including commercially available cocktail of protein inhibitors, can also be used to prevent degradation of the target protein (such as TL1A isoforms and/or proxy markers) of the preparation. The tissue can be homogenized using a homogenizer, many of which are commercially available, according to manufacturer's instructions, to obtain tissue homogenate. The cells can be then separated from the other tissue materials (e.g. extracellular matrix) of the tissue sample. The cells can be further isolated from other tissue materials in the tissue homogenate by centrifugation and taking the precipitant at different centrifugation speed.

For proteins that are in the cells, the proteins can be prepared by lysing the isolated cells. For proteins that are in both the cells and the space outside the cells in the tissue (e.g. extracellular matrix and/or fluid in the tissue), the proteins can be prepared by lysing the tissue homogenate. Such cell or tissue lysis is achieved with lysis buffer, various kinds of which are commercially available (e.g. from THERMO FISCHER SCIENTIFIC or BIO-RAD), according to manufacturer's instructions. Such lysis buffer can include detergent for disrupting the cell membrane, enzymes such as collagenase for breaking down the extracellular matrix, and/or DANase/RNAase for breaking down DNA/RNA. After the lysis step, lysate containing the target protein (e.g. TL1A isoform) is obtained and ready for the next step.

The proteins can be enriched before the determination of the level of the target proteins (e.g. TL1A isoforms and/or proxy markers) in the sample. Such enrichment can be achieved by concentrating or filtering the proteins of certain molecular weight cutoff which the molecular weights of the target proteins (e.g. TL1A isoforms and/or proxy markers) are above, by using membrane with pore size corresponding to the molecular weight cutoff. Such enrichment can also be achieved by dialyzing the lysate using dialysis membrane with molecular weight cutoff which the molecular weights of the target proteins (e.g. TL1A isoforms and/or proxy markers) are above. Alternatively, such enrichment can also be achieved by a step of protein purification and taking the fractions containing the target proteins (e.g. TL1A isoforms and/or proxy markers) after purification (e.g. size exclusion chromatography, ion exchange chromatography, affinity purification, etc).

When the sample containing the target proteins is a bodily fluid instead of a solid tissue, the sample may not need to undergo the homogenization step or the lysis step. The sample can direct undergo the enrichment step.

Alternatively, tissue lysate or bodily fluid can be directly used for determining the level of target proteins (e.g. TL1A isoforms and/or proxy markers).

In some embodiments, when the protein or nucleic acid is directly assayed in situ, for example, immunohistochemistry for protein level determination or in situ hybridization for mRNA level, the sample can be prepared as tissue slices using known and available tissue processor for histology, including the reagents set for such tissue processor.

Accordingly, in one embodiment of the methods provided herein, including in including in Sections 2, 5.1, and 7, the methods further comprising a sample preparation step described in this Section. In one embodiment, the sample preparation step comprises a homogenization step that homogenizes the tissue sample. In another embodiment, the sample preparation step comprises a lysis step that lyses the cells in the sample. In yet another embodiment, the sample preparation step comprises an enrichment step that enriches the target protein and/or nucleic acids in the sample. In a further embodiment, the sample preparation step comprises a step of processing a tissue slide. In one embodiment, the sample preparation step comprises a homogenization step that homogenizes the tissue sample and a lysis step that lyses the cells in the sample. In another embodiment, the sample preparation step comprises a homogenization step that homogenizes the tissue sample, a lysis step that lyses the cells in the sample, and an enrichment step that enriches the target protein and/or nucleic acids in the sample.

### 6. Other Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “and/or” as used in a phrase with a list of members is intended to include all members individually and all combination of full or partial list of members. For example, a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “model,” when used in the context of CDx model or SNP model, or other similar contexts, refer to a system, method, process, and/or composition of (i) a combination of SNPs used in a patient selection criteria. (ii) the description, the identity, or the representations (e.g. mathematical or logical representation) of the SNPs used in the patient selection criteria, and/or (iii) the methods, processes, rules, systems, and/or mathematical operations used to determine the readout of the patient selection criteria based on (i) and (ii), wherein the patient selection criteria is for selecting patients suitable for treatment with an inhibitor of TL1A activity or expression.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215 (3): 403-10; Nucleic Acids Res. 1997 Sep. 1; 25 (17): 3389-402). Percent homology of sequences can be determined using the most recent version of BLAST, as of the filing date of this application. Percent identity of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time).

The terms “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

In some embodiments, the terms “individual” or “subject” are used interchangeably and refer to any animal, including, but not limited to, humans, non-human primates, rodents, and domestic and garne animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition), A “subject in need” of treatment for a particular condition can be a subject having that condition, diagnosed as having that condition, or at risk of developing that condition. In some embodiments, the subject is a “patient.” that has been diagnosed with a disease or condition described herein.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The term, “genotype” as disclosed herein, refers to the chemical composition of polynucleotide sequences within the genome of an individual. In some embodiments, the genotype comprises a single nucleotide polymorphism (SNP) or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

A “polymorphism” as used herein refers to an aberration in (e.g., a mutation), or of (e.g., insertion/deletion), a nucleic acid sequence, as compared to the nucleic acid sequence in a reference population. In some embodiments, the polymorphism is common in the reference population. In some embodiments, the polymorphism is rare in the reference population. In some embodiments, the polymorphism is a single nucleotide polymorphism.

The term, “single nucleotide polymorphism” or SNP as disclosed herein, refers to a variation in a single nucleotide within a polynucleotide sequence. The term should not be interpreted as placing a restriction on a frequency of the SNP in a given population. The variation of an SNP may have multiple different forms. A single form of an SNP is referred to as an “allele.” An SNP can be mono-, bi-, tri, or tetra-allelic. A SNP may include a “risk allele,” a “protective allele,” or neither. By way of example, a reference polynucleotide sequence reading 5′ to 3′ is TTACG. A SNP at allele position 3 (of 5-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. The SNP may be observed in at least 1% of a given population. In some embodiments, the SNP is represented by an “rs” number, which refers to the accession of reference cluster of one more submitted SNPs in the dbSNP bioinformatics database as of the filing date of this patent application, and which is included within a sequence that comprises the total number of nucleobases from 5′ to 3′. In some embodiments, a SNP may be further defined by the position of the SNP (nucleobase) within the dbSNP sequence, the position of which is always with reference to 5′ length of the sequence plus 1. In some embodiments, a SNP is defined as the genomic position in a reference genome and the allele change (e.g. chromosome 7 at position 234,123,567 from G allele to A allele in the reference human genome build 37). In some embodiments, the SNV is defined as the genomic position identified with [brackets] or an “N” in a sequence disclosed herein.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxi.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician. In some cases, therapeutically effective amount of the drug reduces the severity of symptoms of the disease or disorder. In some instances, the disease or disorder comprises inflammatory bowel disease (IBD), Crohn's disease (CD), or ulcerative colitis (UC). In some instances, the IBD, CD, and/or UC are severe or medically refractory forms of the IBD, CD, and/or UC. Non-limiting examples of symptoms of IBD, CD, and/or UC include, but are not limited to, diarrhea, fever, fatigue, abdominal pain, abdominal cramping, inflammation, ulceration, nausea, vomiting, bleeding, blood in stool, reduced appetite, and weight loss.

The term “pharmaceutically acceptable carrier.” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e. g., immune cell).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anit-12 antibody, and anti-Cbir1 flagellin antibody.

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The term “TL1A long isoform,” or “TL1A canonical isoform,” as used herein, refers to long isoform of the protein nationally expressed from TNFSF15 gene or the mRNA encoding such TL1A isoform nationally expressed from TNFSF15 gene, the protein sequence of which comprises the TL1A protein sequence set forth in SEQ ID NO: 2050 or natural variants of the isoform of similar length to SEQ ID NO: 2050.

The term “TL1A short isoform,” as used herein, refers to short isoform of the protein nationally expressed from TNFSF15 gene or the mRNA encoding such TL1A isoform nationally expressed from TNFSF15 gene, the protein sequence of which comprises the TL1A protein sequence set forth in SEQ ID NO: 2052 or natural variants of the isoform of similar length to SEQ ID NO: 2052.

“TL1A isoform ratio” refers to the ratio of TL1A short isoform to TL1A long isoform (TL1Aₛₕₒᵣₜ/TL1A_{long}) or other mathematical derivative of such ratio, in which the mathematical derivative correlates with such ratio in the entire range or a subrange of such ratio. In some embodiments, the mathematical derivative of the ratio correlates with such ratio at least in a subrange that statistically distinguishes the population with high TL1Aₛₕₒᵣₜ/TL1A_{long} ratio from the population with low TL1Aₛₕₒᵣₜ/TL1A_{long} ratio. In some embodiments, the mathematical derivative of the ratio correlates with such TL1Aₛₕₒᵣₜ/TL1A_{long} ratio at least in a subrange that encompasses the mean or median of the TL1Aₛₕₒᵣₜ/TL1A_{long} ratio. In some embodiments, the mathematical derivative of the ratio correlates with such TL1Aₛₕₒᵣₜ/TL1A_{long} ratio at least in a subrange that encompasses the 2nd and 3rd quartile of the TL1Aₛₕₒᵣₜ/TL1A_{long} ratio. In some embodiments, the mathematical derivative of the ratio correlates with such TL1Aₛₕₒᵣₜ/TL1A_{long} ratio at least in a subrange that encompasses the 2nd quartile of the TL1Aₛₕₒᵣₜ/TL1A_{long} ratio. In some embodiments, the mathematical derivative of the ratio correlates with such TL1Aₛₕₒᵣₜ/TL1A_{long} ratio at least in a subrange that encompasses the 3rd quartile of the TL1Aₛₕₒᵣₜ/TL1A_{long} ratio. In some embodiments, the TL1A isoform ratio is calculated as TL1Aₛₕₒᵣₜ/TL1A_{long}. In some embodiments, the TL1A isoform ratio is calculated as ln(TL1Aₛₕₒᵣₜ/TL1A_{long}). In some embodiments, the TL1A isoform ratio is calculated as ln(TL1Aₛₕₒᵣₜ/TL1A_{long}+n), wherein n is a positive integer such as 1.

The section headings used herein are for organizational purposes only and are not to be construed as limiting the subject matter described.

### 7 EXAMPLES

The following examples are included for illustrative purposes only and are not intended to limit the scope of the invention.

### 7.1 Example 1: Design of Humanized Anti-TL1A Antibodies

Two different strategies were employed to identify humanized variants that express well in mammalian cells, preserve TL1A binding, and display high monomeric content.

The first strategy utilized a previously humanized variant, termed ASX, that displays high monomeric content (98%) and expresses well (30 μg/mL in small-scale transient cultures) as a template for additional mutagenesis. However, ASX contains a significant number of murine framework residues, eight heavy chain residues and 7 light chain residues, that may pose an immunogenicity risk. The ASX heavy and light chain templates were used to systematically mutate murine framework residues to human residues corresponding to the most closely related human germline framework. The goal of this strategy was to reduce the total number of murine framework residues while preserving the favorable expression and solubility characteristics of ASX. Because ASX contained 15 murine framework residues there were 2∧15 (32,768)distinct variants (restricting each position to either the murine or the human residue) that could be made and tested.

The second strategy utilized a previously humanized variant, termed c34, that expresses well (17 μg/mL in small-scale transient cultures) and contains CDRs optimized for binding within a fully human germline framework, as a template for additional mutagenesis. Large-scale expression of c34 unexpectedly resulted in a sub-optimal monomeric content (55-60%). The c34 heavy and light chain templates were used to systematically mutate certain framework residues to murine residues corresponding to the original murine antibody framework. The goal of this strategy was to improve the solubility of c34 (monomeric content) through the introduction of as few murine framework residues as possible (minimizing potential immunogenicity risks) while preserving the favorable expression characteristics of c34.

For both strategies, the initial approach was to scan differing framework residues, one at a time, and express and characterize the variants. Thus, human framework residues were introduced into variant ASX where it differed from c34 and conversely, murine framework mutations were introduced into variant c34 where it differed from ASX. The initial scan identified certain framework and CDR residues that had minimal impact on the characteristics displayed by the template antibody while other mutations had a more dramatic impact, favorable in some cases and unfavorable in others. The information gained from the positional scan was subsequently used in an iterative and combinatorial fashion, to identify multiple variants with favorable characteristics. Importantly, by applying a stepwise, iterative and combinatorial approach the beneficial variants were identified without necessitating the expression and characterization of 32,768 distinct variants.

In certain cases, mutation of the first residue of the heavy chain from glutamine to aspartic acid or glutamic acid was evaluated, alone or in combination with other mutations.

In addition, for both strategies certain CDR residues were also mutated to determine the impact on expression and solubility. For example, a limited number of mutations in HCDR2, HCDR3 and LCDR3 were examined. Similar to the approach used with frameworks, the mutations were predominantly restricted to the original murine CDR residue or mutations that were previously identified as enhancing binding affinity.

Finally, for both strategies “shuffling” of heavy and light chains was used. Specifically, certain human light chains containing few murine framework residues and having a favorable impact on expression of antibody with higher monomeric content were identified early in the process and these were paired with various engineered heavy chains in order to accelerate the process of identifying suitable variants.

TABLE 10
Sequences of Certain Designed anti-TL1A Antibodies

| | Heavy Chain | Light Chain |
|---|---|---|
| | Variable Region | Variable Region |
| Antibody | SEQ ID NO | SEQ ID NOS |
| A15 | 108 | 203 |
| A29 | 108 | 205 |
| A30 | 108 | 204 |
| A31 | 136 | 205 |
| A32 | 137 | 205 |
| A33 | 137 | 202 |
| A34 | 107 | 208 |
| A35 | 138 | 208 |
| A36 | 139 | 208 |
| A37 | 140 | 208 |
| A38 | 141 | 208 |
| A39 | 142 | 208 |
| A40 | 143 | 208 |
| A41 | 115 | 208 |
| A42 | 144 | 208 |
| A43 | 145 | 208 |
| A44 | 146 | 208 |
| A45 | 120 | 208 |
| A46 | 147 | 208 |
| A47 | 148 | 208 |
| A48 | 108 | 210 |
| A49 | 108 | 211 |
| A50 | 108 | 212 |
| A51 | 108 | 213 |
| A52 | 108 | 214 |
| A53 | 146 | 208 |
| A54 | 149 | 208 |
| A55 | 109 | 208 |
| A56 | 108 | 215 |
| A57 | 150 | 202 |
| A58 | 125 | 202 |
| A59 | 117 | 202 |
| A60 | 151 | 202 |
| A61 | 152 | 202 |
| A62 | 153 | 202 |
| A63 | 154 | 202 |
| A64 | 121 | 202 |
| A65 | 128 | 202 |
| A66 | 155 | 202 |
| A67 | 122 | 202 |
| A68 | 123 | 202 |
| A69 | 156 | 202 |
| A70 | 157 | 202 |
| A71 | 158 | 202 |
| A72 | 131 | 202 |
| A73 | 157 | 205 |
| A74 | 158 | 205 |
| A75 | 131 | 205 |
| A76 | 159 | 202 |
| A77 | 160 | 202 |
| A78 | 124 | 202 |
| A79 | 107 | 208 |
| A81 | 139 | 208 |
| A82 | 140 | 208 |
| A83 | 144 | 208 |
| A85 | 136 | 209 |
| A86 | 136 | 216 |
| A87 | 136 | 217 |
| A88 | 136 | 218 |
| A89 | 136 | 219 |
| A90 | 136 | 220 |
| A91 | 133 | 202 |
| A92 | 161 | 202 |
| A93 | 162 | 202 |
| A94 | 124 | 202 |
| A95 | 131 | 205 |
| A96 | 128 | 205 |
| A97 | 121 | 202 |
| A98 | 122 | 202 |
| A99 | 123 | 202 |
| A100 | 107 | 204 |
| A101 | 140 | 204 |
| A102 | 115 | 204 |
| A103 | 120 | 204 |
| A104 | 139 | 204 |
| A105 | 143 | 204 |
| A107 | 108 | 202 |
| A108 | 156 | 205 |
| A109 | 133 | 205 |
| A110 | 125 | 205 |
| A111 | 150 | 205 |
| A112 | 117 | 205 |
| A113 | 124 | 205 |
| A114 | 121 | 205 |
| A115 | 122 | 205 |
| A116 | 123 | 205 |
| A117 | 151 | 205 |
| A118 | 153 | 205 |
| A119 | 159 | 205 |
| A120 | 154 | 205 |
| A121 | 163 | 204 |
| A122 | 113 | 204 |
| A123 | 112 | 204 |
| A124 | 164 | 204 |
| A125 | 105 | 204 |
| A126 | 114 | 204 |
| A127 | 118 | 204 |
| A128 | 111 | 204 |
| A129 | 110 | 204 |
| A130 | 121 | 205 |
| A132 | 128 | 206 |
| A133 | 121 | 206 |
| A134 | 122 | 206 |
| A135 | 133 | 206 |
| A136 | 125 | 206 |
| A137 | 121 | 207 |
| A138 | 122 | 207 |
| A139 | 110 | 207 |
| A140 | 110 | 202 |
| A141 | 111 | 207 |
| A142 | 111 | 202 |
| A143 | 136 | 202 |
| A144 | 111 | 204 |
| A145 | 133 | 201 |
| A146 | 125 | 201 |
| A147 | 117 | 201 |
| A148 | 121 | 201 |
| A149 | 122 | 201 |
| A150 | 128 | 201 |
| A151 | 124 | 201 |
| A152 | 131 | 201 |
| A153 | 133 | 205 |
| A154 | 125 | 205 |
| A155 | 121 | 205 |
| A156 | 122 | 205 |
| A157 | 104 | 204 |
| A158 | 101 | 204 |
| A159 | 119 | 204 |
| A160 | 102 | 204 |
| A161 | 165 | 204 |
| A162 | 106 | 204 |
| A163 | 166 | 204 |
| A164 | 167 | 204 |
| A165 | 139 | 205 |
| A166 | 146 | 205 |
| A167 | 120 | 205 |
| A168 | 147 | 205 |
| A169 | 126 | 205 |
| A170 | 135 | 205 |
| A171 | 168 | 205 |
| A172 | 130 | 205 |
| A173 | 127 | 205 |
| A174 | 132 | 205 |
| A175 | 126 | 201 |
| A176 | 135 | 201 |
| A177 | 168 | 201 |
| A178 | 130 | 201 |
| A179 | 127 | 201 |
| A180 | 132 | 201 |
| A181 | 107 | 202 |
| A182 | 138 | 202 |
| A183 | 140 | 202 |
| A184 | 145 | 202 |
| A185 | 147 | 202 |
| A186 | 144 | 202 |
| A187 | 120 | 202 |
| A188 | 115 | 202 |
| A189 | 146 | 202 |
| A190 | 141 | 202 |
| A191 | 142 | 202 |
| A192 | 143 | 202 |
| A193 | 109 | 205 |
| A194 | 103 | 205 |
| A195 | 169 | 205 |
| A196 | 129 | 205 |
| A197 | 116 | 205 |
| A198 | 134 | 205 |
| A199 | 109 | 201 |
| A200 | 103 | 201 |
| A201 | 169 | 201 |
| A202 | 129 | 201 |
| A203 | 116 | 201 |
| A204 | 134 | 201 |
| A205 | 109 | 202 |
| A206 | 103 | 202 |
| A207 | 169 | 202 |
| A208 | 129 | 202 |
| A209 | 116 | 202 |
| A210 | 134 | 202 |
| A211 | 108 | 201 |
| A212 | 107 | 201 |
| A213 | 106 | 201 |
| A214 | 111 | 201 |
| A215 | 110 | 201 |
| A216 | 112 | 201 |
| A217 | 101 | 201 |
| A218 | 119 | 201 |
| A219 | 104 | 201 |
| A220 | 102 | 205 |
| A221 | 105 | 201 |
| A222 | 114 | 201 |
| A223 | 103 | 202 |
| A224 | 116 | 201 |
| A500 | 301 | 303 |
| A501 | 302 | 303 |

As used herein, reference to A (number), refers to an antibody of this table. For instance, A15 used herein refers to A15 in Table 10.

### 7.2 Example 2: Generation and Characterization of Humanized Anti-Tl1a Antibodies

Humanized anti-TL1A antibodies designed in Example 1 were prepared and characterized.

### Cloning of Humanized Antibodies

DNA encoding leader sequence and the heavy and light chain variable regions of humanized variants of interest was cloned into pFuse1-hIgG1-Fc1 (InvivoGen) and pFuse2-CLig-hk (InvivoGen), respectively. Two distinct humanized heavy chain templates, termed ASX-HC and c34-HC, and four distinct humanized light chain templates, termed ASX-LC, cH3-1, c34-LC, cXL3-13-LC and cXL3-15-LC were all cloned.

In order to introduce mutations into the templates, the QuickChange Site Directed Mutagenesis Kit (Agilent, cat. #200518) was used per manufacturer's directions. Briefly, mutagenesis was performed using miniprep double-stranded plasmid DNA, two synthetic oligonucleotides primers containing the desired mutation, PfuTurbo® DNA polymerase and a temperature cycler. Following temperature cycling, the product was treated with Dpn 1. The nicked vector DNA containing the mutation(s) of interest was used to transform bacteria. Subsequently, colonies were picked, the DNA was sequenced to confirm mutagenesis and was subsequently used for transfection of mammalian FreeStyle 293-F cells.

### Antibody Expression

Small-scale (3 mL, 6-well) expression of variants in FreeStyle 293-F cells was performed in the following manner. One or two days prior to transfection cells were passaged so that the density would be >1×10⁶ cells/mL on the day of the transfection. Typically, this meant passaging at 6-7×105 cells/mL one day prior or 4×105 cells/mL two days prior. Transfections were only performed with cell viability >90%. On the day of the transfection Opti-MEM media was warmed to 37° C. and cells were resuspended to 1.1×106 cells/mL, using 3.3×106 cells per 3 mL transfection. A total of 3 ug DNA was used for each transfection. Briefly, the transfections used heavy and light chain plasmid at a heavy chain: light chain ratio of 1:3. For 3 mL transfections, 4 uL 293fectin was added to 96 uL Opti-MEM, combined with 100 uL DNA mixture, and incubated at 25° C. for 20-30 minutes. Subsequently, this mixture was added dropwise to 2.8 mL cells and the plate was transferred to an incubator and placed on a rotating platform at 175 rpm for up to 120 hours. After 96-120 hours, transfection supernatants were collected by centrifuging the transfected cells and supernatant at 1200 rpm for 5 min. The supernatant was transferred to a clean tube and centrifuged again at 3900 rpm for 10 min to remove any remaining cell debris. The supernatant was filtered through a 0.45 mm PES syringe filter and stored at 4° C. until the next step.

### Quantitation of Antibody Expression

Antibody expression was quantitated by ELISA. Briefly, a Corning Costar 3366 96-well round bottom high bind plate was coated with 50 mL anti-kappa (2 μg/mL) in PBS overnight at 4° C. The plate was washed 3× with PBS-0.05% Tween 20 (PBS-T) and was blocked with 100 μL 1% BSA/PBS for 1 h at 25° C. The block was removed, and culture supernatant diluted 5-fold was added and serially diluted 2-fold across the plate. Every plate also contained an IgG standard diluted serially 3-fold beginning at 1 μg/mL. Samples were incubated for 1 h at 25° C., the plate was washed three times with PBS-T, and 50 μL anti-Fc HRP secondary (Southern Biotech #2048-05), diluted 1:4000 in BSA/PBS was added for 1 h at 25° C. The plate was washed three times with PBS-T and developed for up to 15 min following the addition of 50 μL Ultra TMB ELISA substrate (Thermo #34028). The reaction was terminated by the addition of 50 μL 2 N H₂SO₄ and the A450 nm was measured. Antibody expression levels obtained from 3 mL scale transfections are shown in Table 11.

TABLE 11
Expression, Binding, and Analytical SEC Characterization
of anti-TL1A Antibodies (ND, not determined)

| Var- | Expression | KD | % | Murine | HC | LC |
|---|---|---|---|---|---|---|
| iant | (ug/mL) | (pM) | Monomer | FR | Template | Template |
| 15 | 21 | ND | 87 | 8 | ASX | cH3-1 |
| 29 | 18 | 65 | 65 | 10 | ASX | c34 |
| 30 | 29 | 77 | 90 | 8 | ASX | cXL3-13 |
| 31 | 11 | 92 | 73 | 2 | c34 | c34 |
| 32 | 10 | 111 | 78 | 2 + D | c34 | c34 |
| 33 | 21 | 81 | 54 | 0 + D | c34 | c34 |
| 34 | 35 | <50 | 97 | 14 | ASX | ASX |
| 35 | 36 | 72 | 91 | 14 | ASX | ASX |
| 36 | 40 | <50 | 87 | 13 | ASX | ASX |
| 37 | 40 | 34 | 95 | 14 | ASX | ASX |
| 38 | 28 | 103 | 75 | 14 | ASX | ASX |
| 39 | 15 | 125 | 83 | 14 | ASX | ASX |
| 40 | 30 | <50 | 87 | 13 | ASX | ASX |
| 41 | 20 | 16 | 96 | 14 | ASX | ASX |
| 42 | 30 | <50 | 88 | 14 | ASX | ASX |
| 43 | 18 | 51 | 90 | 14 | ASX | ASX |
| 44 | ND | ND | ND | 13 | ASX | ASX |
| 45 | 15 | 85 | 90 | 13 | ASX | ASX |
| 46 | 27 | 63 | 72 | 13 | ASX | ASX |
| 47 | 18 | 82 | 78 | 12 | ASX | ASX |
| 48 | 22 | 76 | 92 | 14 | ASX | ASX |
| 49 | 26 | 92 | 65 | 13 | ASX | ASX |
| 50 | 33 | 19 | 94 | 14 | ASX | ASX |
| 51 | 16 | <50 | 93 | 14 | ASX | ASX |
| 52 | 29 | 27 | 91 | 13 | ASX | ASX |
| 53 | 26 | 126 | 84 | 13 | ASX | ASX |
| 54 | 25 | 83 | 94 | 15 + D | ASX | ASX |
| 55 | 22 | 91 | 99 | 15 + E | ASX | ASX |
| 56 | 15 | 116 | 71 | 14 | ASX | ASX |
| 57 | 20 | 191 | 59 | 1 | c34 | c34 |
| 58 | 9 | 112 | 67 | 1 | c34 | c34 |
| 59 | 11 | 136 | 78 | 2 | c34 | c34 |
| 60 | 19 | 168 | 57 | 0 | c34 | c34 |
| 61 | 15 | 127 | 44 | 1 | c34 | c34 |
| 62 | 21 | 150 | 58 | 1 | c34 | c34 |
| 63 | 20 | 132 | 52 | 0 | c34 | c34 |
| 64 | 2 | 90 | 97 | 0 | c34 | c34 |
| 65 | 7 | 97 | 69 | 1 | c34 | c34 |
| 66 | 19 | 150 | 49 | 1 | c34 | c34 |
| 67 | 4 | 89 | 97 | 1 | c34 | c34 |
| 68 | 2 | 74 | 92 | 1 | c34 | c34 |
| 69 | 12 | 136 | 64 | 0 + E | c34 | c34 |
| 70 | 15 | 149 | 54 | 1 | c34 | c34 |
| 71 | 18 | 150 | 55 | 2 | c34 | c34 |
| 72 | 13 | 159 | 61 | 3 | c34 | c34 |
| 73 | 8 | 128 | 71 | 3 | c34 | c34 |
| 74 | 10 | 141 | 70 | 4 | c34 | c34 |
| 75 | 8 | 259 | 95 | 5 | c34 | c34 |
| 76 | 19 | ND | 50 | 0 | c34 | c34 |
| 77 | 12 | ND | 50 | 2 | c34 | c34 |
| 78 | 3 | ND | 86 | 2 | c34 | c34 |
| 79 | 42 | ND | 98 | 14 | ASX | ASX |
| 81 | 31 | ND | 88 | 13 | ASX | ASX |
| 82 | 26 | ND | 92 | 14 | ASX | ASX |
| 83 | 29 | ND | 74 | 14 | ASX | ASX |
| 85 | 25 | 130 | 49 | 1 | c34 | c34 |
| 86 | 26 | 129 | 55 | 1 | c34 | c34 |
| 87 | 26 | 121 | 52 | 1 | c34 | c34 |
| 88 | 9 | 81 | 63 | 2 | c34 | c34 |
| 89 | 31 | 117 | 55 | 1 | c34 | c34 |
| 90 | 19 | 107 | 53 | 1 | c34 | c34 |
| 91 | 14 | 132 | 63 | 1 | c34 | c34 |
| 92 | 20 | 121 | 49 | 1 | c34 | c34 |
| 93 | 12 | 117 | 63 | 2 | c34 | c34 |
| 94 | 5 | 81 | 91 | 2 | c34 | c34 |
| 95 | 13 | 105 | 92 | 5 | c34 | c34 |
| 96 | 7 | 95 | 99 | 3 | c34 | c34 |
| 97 | 2 | 71 | 97 | 0 | c34 | c34 |
| 98 | 7 | 140 | 98 | 1 | c34 | c34 |
| 99 | 3 | 102 | 95 | 1 | c34 | c34 |
| 100 | 39 | 84 | 84 | 7 | ASX | cXL3-13 |
| 101 | 23 | 96 | 81 | 7 | ASX | cXL3-13 |
| 102 | 19 | 104 | 75 | 7 | ASX | cXL3-13 |
| 103 | 11 | 107 | 90 | 6 | ASX | cXL3-13 |
| 104 | 26 | 108 | 70 | 6 | ASX | cXL3-13 |
| 105 | 23 | 110 | 58 | 6 | ASX | cXL3-13 |
| 107 | 55 | 71 | 85 | 8 | ASX | c34 |
| 108 | 9 | 55 | 83 | 2 + E | c34 | c34 |
| 109 | 9 | 50 | 96 | 3 | c34 | c34 |
| 110 | 7 | 56 | 95 | 3 | c34 | c34 |
| 111 | 17 | 68 | 61 | 3 | c34 | c34 |
| 112 | 6 | 54 | 93 | 4 | c34 | c34 |
| 113 | 2 | 50 | 99 | 4 | c34 | c34 |
| 114 | 1 | 51 | 99 | 2 | c34 | c34 |
| 115 | 3 | 58 | 99 | 3 | c34 | c34 |
| 116 | 1 | 53 | 99 | 3 | c34 | c34 |
| 117 | 16 | 94 | 80 | 2 | c34 | c34 |
| 118 | 21 | 83 | 70 | 3 | c34 | c34 |
| 119 | 15 | 87 | 77 | 2 | c34 | c34 |
| 120 | 12 | 85 | 64 | 2 | c34 | c34 |
| 121 | 24 | 106 | 77 | 6 | ASX | cXL3-13 |
| 122 | 22 | 112 | 85 | 6 | ASX | cXL3-13 |
| 123 | 18 | 104 | 76 | 5 | ASX | cXL3-13 |
| 124 | 21 | 91 | 83 | 6 | ASX | cXL3-13 |
| 125 | 10 | 116 | 98 | 6 | ASX | cXL3-13 |
| 126 | 4 | 123 | 99 | 5 | ASX | cXL3-13 |
| 127 | 8 | 70 | 94 | 6 | ASX | cXL3-13 |
| 128 | 17 | 111 | 84 | 4 | ASX | cXL3-13 |
| 129 | 17 | 99 | 92 | 5 | ASX | cXL3-13 |
| 130 | 1 | 75 | 99 | 2 | c34 | c34 |
| 132 | 6 | 62 | 99 | 2 | c34 | cXL3-13 |
| 133 | 1 | 58 | 99 | 1 | c34 | cXL3-13 |
| 134 | 3 | 55 | 99 | 2 | c34 | cXL3-13 |
| 135 | 7 | 56 | 74 | 2 | c34 | cXL3-13 |
| 136 | 6 | 53 | 84 | 2 | c34 | cXL3-13 |
| 137 | 2 | 50 | 96 | 0 | c34 | cXL3-15 |
| 138 | 5 | 69 | 99 | 1 | c34 | cXL3-15 |
| 139 | 35 | 74 | 78 | 5 | ASX | cXL3-15 |
| 140 | 26 | 73 | 75 | 5 | ASX | c34 |
| 141 | 27 | 108 | 81 | 4 | ASX | cXL3-15 |
| 142 | 25 | 126 | 68 | 4 | ASX | c34 |
| 143 | 16 | 85 | 57 | 0 | c34 | c34 |
| 144 | ND | ND | ND | 4 | ASX | cXL3-13 |
| 145 | 20 | 70 | 78 | 2 | c34 | c34 |
| 146 | 25 | 65 | 84 | 2 | c34 | c34 |
| 147 | 26 | 63 | 87 | 3 | c34 | c34 |
| 148 | 2 | 46 | 98 | 1 | c34 | c34 |
| 149 | 7 | 48 | 99 | 2 | c34 | c34 |
| 150 | 15 | 59 | 83 | 2 | c34 | c34 |
| 151 | 5 | 57 | 96 | 3 | c34 | c34 |
| 152 | 36 | 58 | 73 | 4 | c34 | c34 |
| 153 | 9 | 49 | 97 | 3 | c34 | c34 |
| 154 | 8 | 66 | 92 | 3 | c34 | c34 |
| 155 | 1 | 67 | 99 | 2 | c34 | c34 |
| 156 | 2 | 94 | 99 | 3 | c34 | c34 |
| 157 | 6 | 69 | 93 | 4 | ASX | cXL3-13 |
| 158 | 6 | 66 | 91 | 3 | ASX | cXL3-13 |
| 159 | 4 | 69 | 99 | 4 | ASX | cXL3-13 |
| 160 | 7 | 94 | 99 | 4 | ASX | cXL3-13 |
| 161 | 11 | 72 | 59 | 4 | ASX | cXL3-13 |
| 162 | 9 | 75 | 79 | 3 | ASX | cXL3-13 |
| 163 | 22 | 51 | 60 | 4 | ASX | cXL3-13 |
| 164 | 23 | 58 | 61 | 4 | ASX | cXL3-13 |
| 165 | 19 | 59 | 53 | 8 | ASX | c34 |
| 166 | 13 | 57 | 76 | 8 | ASX | c34 |
| 167 | 9 | 42 | 96 | 8 | ASX | c34 |
| 168 | 16 | 62 | 85 | 8 | ASX | c34 |
| 169 | 8 | 47 | 90 | 3 | c34 | c34 |
| 170 | 9 | 49 | 93 | 3 | c34 | c34 |
| 171 | 13 | 50 | 80 | 5 | c34 | c34 |
| 172 | 7 | 40 | 96 | 3 | c34 | c34 |
| 173 | 4 | 40 | 99 | 4 | c34 | c34 |
| 174 | 4 | 43 | 98 | 4 | c34 | c34 |
| 175 | 31 | 45 | 86 | 2 | c34 | c34 |
| 176 | 18 | 48 | 80 | 2 | c34 | c34 |
| 177 | 35 | 52 | 67 | 4 | c34 | c34 |
| 178 | 18 | 43 | 85 | 2 | c34 | c34 |
| 179 | 16 | 79 | 93 | 3 | c34 | c34 |
| 180 | 17 | 58 | 94 | 3 | c34 | c34 |
| 181 | 46 | 60 | 87 | 7 | ASX | c34 |
| 182 | 39 | 67 | 74 | 7 | ASX | c34 |
| 183 | 38 | 65 | 82 | 7 | ASX | c34 |
| 184 | 30 | 61 | 73 | 7 | ASX | c34 |
| 185 | 30 | 56 | 66 | 6 | ASX | c34 |
| 186 | 38 | 67 | 66 | 7 | ASX | c34 |
| 187 | 27 | 56 | 72 | 6 | ASX | c34 |
| 188 | 31 | 63 | 87 | 7 | ASX | c34 |
| 189 | 44 | 76 | 71 | 6 | ASX | c34 |
| 190 | 32 | 57 | 69 | 7 | ASX | c34 |
| 191 | 21 | 57 | 80 | 7 | ASX | c34 |
| 192 | 27 | 55 | 70 | 6 | ASX | c34 |
| 193 | 16 | 55 | 68 | 10 + E | ASX | c34 |
| 194 | 16 | 51 | 87 | 9 + E | ASX | c34 |
| 195 | 12 | 56 | 82 | 5 + E | c34 | c34 |
| 196 | 7 | 54 | 97 | 3 + E | c34 | c34 |
| 197 | 7 | 54 | 97 | 3 + E | c34 | c34 |
| 198 | 9 | 53 | 95 | 3 + E | c34 | c34 |
| 199 | 28 | 50 | 93 | 9 + E | ASX | c34 |
| 200 | 24 | 52 | 99 | 8 + E | ASX | c34 |
| 201 | 25 | 58 | 82 | 4 + E | c34 | c34 |
| 202 | 13 | 59 | 87 | 2 + E | c34 | c34 |
| 203 | 18 | 62 | 89 | 2 + E | c34 | c34 |
| 204 | 11 | 53 | 84 | 2 + E | c34 | c34 |
| 205 | 27 | 55 | 86 | 8 + E | ASX | c34 |
| 206 | 20 | 50 | 98 | 7 + E | ASX | c34 |
| 207 | ND | ND | ND | 3 + E | c34 | c34 |
| 208 | ND | ND | ND | 1 + E | c34 | c34 |
| 209 | 14 | 58 | 66 | 1 + E | c34 | c34 |
| 210 | 15 | 70 | 61 | 1 + E | c34 | c34 |
| 211 | 42 | 58 | 96 | 9 | ASX | c34 |
| 212 | 33 | 50 | 99 | 8 | ASX | c34 |
| 213 | 29 | 49 | 99 | 4 | ASX | c34 |
| 214 | 27 | 51 | 97 | 5 | ASX | c34 |
| 215 | 20 | 48 | 77 | 6 | ASX | c34 |
| 216 | 24 | 49 | 97 | 6 | ASX | c34 |
| 217 | 15 | 43 | 99 | 4 | ASX | c34 |
| 218 | 13 | 51 | 96 | 5 | ASX | c34 |
| 219 | 21 | 50 | 99 | 5 | ASX | c34 |
| 220 | 18 | 50 | 99 | 6 | ASX | c34 |
| 221 | 23 | 51 | 98 | 7 | ASX | c34 |
| 222 | 29 | 60 | 96 | 6 | ASX | c34 |
| 223 | 19 | 62 | 98 | 7 + E | ASX | c34 |
| 224 | 15 | 76 | 92 | 2 + E | c34 | c34 |

### Antibody Binding to Human TL1A

Antibody binding to human TL1A (Fitzgerald #30R-AT070) was quantitated by ELISA. Briefly, a Corning Costar 3366 96-well round bottom high bind plate was coated with 50 μL TL1A (1 μg/mL) in PBS overnight at 4° C. The plate was washed 3× with PBS-0.05% Tween 20 (PBS-T) and was blocked with 100 μL 1% BSA/PBS for 1 h at 25° C. The block was removed, and culture supernatant diluted 5-fold was added and serially diluted 2-fold across the plate. Samples were incubated for 1 h at 25° C., the plate was washed three times with PBS-T, and 50 uL anti-Fc HRP secondary, diluted 1:4000 in BSA/PBS was added for 1 h at 25° C. The plate was washed three times with PBS-T and developed for up to 15 min following the addition of 50 μL Ultra TMB ELISA substrate. The reaction was terminated by the addition of 50 μL 2 N H₂SO₄ and the A450 nm was measured. The antibody affinities, as determined by ELISA titration against human TL1A using unpurified culture supernatants, is shown in Table 11.

### Purification of Antibodies

Antibodies were purified from culture supernatants in a single step using Dynabeads Protein A (ThermoFisher Scientific, cat. #10002D). First, culture supernatants were concentrated per manufacturer's instructions using an Amicon Ultra-4 Centrifugal Filter Unit (30,000 MWCO; Millipore Sigma, cat. #C7719). The Dynabeads were resuspended by gentle vortexing and 100 uL were transferred to an Eppendorf tube. Using a magnet to retain the beads, the storage buffer was removed, and the beads were washed with 0.5 mL of 20 mM sodium phosphate, 150 mM NaCl, pH 7.4 (EB, Equilibration Buffer). A total of up to 24 μg of IgG from culture supernatant was added to the beads and mixed gently until the beads were resuspended. When necessary, antibody superatants were diluted with EB. The tubes were placed sideways on a shaking platform and mixed for 10 min at 25° C. at 500 rpm. Subsequently, the beads were collected at the bottom of the tube using a microfuge at 10,000 rpm for 30 sec. Using a magnet to retain the beads, the supernatant was removed. The beads were washed once with 0.5 mL of 20 mM sodium phosphate, 500 mM NaCl, pH 7.4 followed by another wash with 50 mM sodium phosphate, pH 6.0. The beads were collected at the bottom of the tube using a microfuge at 10,000 rpm for 30 sec. Purified antibody was eluted from the beads using 20 μL 50 mM sodium acetate. pH 3.5 with gentle mixing for 2 min at 25° C. Using a magnet to retain the beads, the eluate was transferred to a fresh tube containing 1.1 μL 1 M Tris, pH 8.5 to neutralize the pH of the sample. This sample was then centrifuged at 10,000 rpm for 2 min and transferred to a fresh tube to ensure removal of residual Dynabeads. The concentration of the purified sample was determined using a DeNovix DS-11 Spectrophotometer/Fluorometer, buffer blank, and a mass extinction coefficient of 13.70 at 280 nm for a 1% IgG solution.

### Size Exclusion Chromatography

The antibodies were analyzed by size exclusion chromatography (SEC) to determine percent monomer and identify any large molecular weight aggregate contaminant species. A total volume of 15 μL of protein A purified antibodies at a concentration of 0.1-1 μg/uL were analyzed using a Waters SEC column (Acquity UPLC BEH SEC, 200 Å, 1.7 μm, 4.6×150 mm) on a Shimadzu UPLC instrument at a flow rate of 0.2 mL/min and a column oven temperature of 30° C. Standard PBS was used as the mobile phase and absorbance at 280 nm was used to monitor protein elution. For some antibody clones tested that demonstrated non-symmetrical elution profiles, PBS buffer supplemented with 350 mM NaCl at pH 6.0 was utilized to reduced non-specific interactions with the column matrix. The percent main peak (monomer) value was calculated using the Shimadzu software. Representative sample profiles are shown in FIGS. 5-7. The monomeric content of purified antibody variants is shown in Table 11.

### 7.3 Example 3: Abrogation of Effector Function

In certain cases, it might be beneficial to reduce the potential effector function of the antibodies. Multiple strategies to diminish effector function have been described, including point mutations to ablate FcγR and C1q binding, cross-subclass Fc designs to eliminate FeγR and C1q binding, and glycoengineering to ablate FcγR and C1q binding. Representative examples are highlighted in Table 12.

TABLE 12
Representative Approaches to Abrogating Effector Function

| Mutation(s) | Effect |
|---|---|
| E233P | Decreases binding to FcγRI, II, III |
| S228P, L235E SPLE in IgG4 | Decreases binding to FcγRI |
| L235E | Decreases binding to FcγRs |
| L234A, L235A | Decreases binding to FcγRI, II, III |
| L234A, L235A, G237A | Decreases binding to FcγRI, II, III, C1q |
| L234A, L235A, P329G | Decreases binding to FcγRI, II, III, C1q |
| L234F, L235E, P331S | Decreases binding to FcγRI, II, III, C1q |
| L234A, L235E, G237A | Decreases binding to FcγRI, II, III, C1q |
| L234A, L235E, G237A, P331S | Decreases binding to FcγRI, II, III, C1q |
| L234A, L235A, G237A, P238S, H268A, | Decreases binding to FcγRI, IIa, IIb, IIIa |
| A330S, P331S (IgG1σ) | |
| L234A, L235A, P329A | Decreases binding to FcγRI, II, III, C1q |
| G236R, L328R | Decreases binding to FcγRI, II, III |
| G237A | Decreases binding to FcγRII |
| F241A | Decreases binding to C1q |
| V264A | Decreases binding to C1q |
| D265A | Decreases binding to FcγRI, II, III |
| D265A, N297A | Decreases binding to FcγRI, II, III, C1q |
| D265A, N297G | Decreases binding to FcγRI, II, III, C1q |
| D270A | Decreases binding to C1q |
| N297A, G, D, Q | Elimination of N-linked glycosylation |
| | Decreases binding to FcγRI, II, III, C1q |
| P329A, G, R | Decreases binding to C1q |
| A330L | Decreases binding to C1q |
| P331A, S | Diminished C1q binding |
| IgG2 | Decreases binding to FcγRs |
| IgG4 | Decreases binding to FcγRs; |
| | Does not activate complement system |
| S228P | Prevent IgG4 Fab arm exchange |
| S228P, F234A, L235A (IgG4) | Decreases binding to FcγRI, IIa, IIIa |
| IgG2-IgG4 cross-subclass (IgG2/G4) | Decreases binding to FcγRI, II, III, C1q |
| IgG2-IgG3 cross-subclass | Decreases binding to FcγRs; |
| | Decreases binding to C1q |
| H268Q, V309L, A330S, P331S (IgG2m4) | Decreases binding to FcγRI, II, III, C1q |
| V234A, G237A, P238S, H268A, V309L, | Decreases binding to FcγRI, IIa, IIb, IIIa, C1q |
| A330S, P331S (IgG2 σ) | |
| High mannose glycosylation | Decreases binding to C1q |

In order to express antibodies with abrogated effector function, the light chain variable regions of the antibodies disclosed in Example 2 and Table 10 are cloned with a kappa light chain constant region, while the heavy chain variable regions are cloned with a modified IgG1 heavy chain backbone, or a modified IgG2 backbone, or a modified IgG4 backbone, or an unmodified IgG2 or IgG4 backbone, such as those disclosed in Table 3 or elsewhere.

The impact of the various Fc engineering approaches on CDC activity can be assessed using C1q binding and C3 fixation assays. Purified antibodies are diluted in PBS and serial dilutions are plated on a microtiter plate for 12-18 h at 4° C. The plates are blocked with 5% gelatin/PBS containing 1% (v/v) Tween-20 for 1 h at 25° C. Subsequently, the plates are incubated with 10% (v/v) human sera in PBS and C1q binding is detected using 1:500 dilution of HRP-conjugated rabbit anti-C1q(Bioss Inc.) in PBS containing 1% (v/v) Tween-20. To test C3 fixation, a 1:1000 dilution of rabbit anti C3 (abcam) is used followed by a 1:2000 dilution of HRP-conjugated chicken anti-rabbit IgG (abcam). The plates are developed as described for antibody quantitation assays in Example 1. EC50 values are calculated by fitting the data to a log (agonist) vs. response-variable slope (four parameter) model using GraphPad Prism (Sunnyvale, CA).

Additionally, the variants may be characterized for the binding of isolated C1q. MaxiSorp 384-well plates (Thermo Scientific, Nunc) are coated with serially diluted antibodies in 50 mM carbonate buffer, pH 9.6 (coat buffer), for 12-18 h at 4° C. Plates are washed with phosphate buffered saline (PBS) containing 0.05% polysorbate 20, pH 7.4 and blocked with PBS containing 0.5% BSA, 0.05% polysorbate 20, 15 ppm Proclin and 10% Blocker Casein (ThermoScientific), pH 7.4. After 1-hour incubation at 25° C., plates are washed. Human C1q (Quidel, San Diego, CA) in the same buffer is added and incubated for 1.5 hour. Bound C1q is detected by adding 20 ng/ml biotinylated mouse anti-mouse C1q(Hycult biotech; cross reacting with human C1q) for 1.5 hour followed by horseradish peroxidase (HRP)-conjugated streptavidin (GE Healthcare Life Sciences) for 1 hour. To check for coating efficiency, some coated wells receive buffer only for the first two incubation steps and receive goat anti-human Fab′2-HRP when the wells used for measuring C1q binding received streptavidin-HRP. Plates are washed after each incubation step. Peroxidase activity is detected with substrate 3, 3′, 5, 5′-tetramethyl benzidine (TMB) (Kirkegaard & Perry Laboratories). The reaction is stopped with IM phosphoric acid and absorbance is measured at 450 nm. Dose-response binding curves are fitted with a four-parameter model and EC50 values are calculated using GraphPad Prism (Sunnyvale, CA).

The impact of the various Fe engineering approaches on ADCC activity is assessed using soluble FcγR receptor binding ELISAs. Soluble human FcγR1, FcγRIIb and FcγRIII (binding affinity to both the F158 and V158 polymorphic forms of FcγRIII is assessed) are expressed as recombinant fusion proteins with Gly-His6-glutathione-S-transferase (GST) at the C-terminus of the extracellular domain of the receptor. MaxiSorp 384-well plates are coated with 1 μg/ml human FcγR in coat buffer. Plates are washed and blocked with PBS containing 0.5% BSA, 15 ppm Proclin, pH 7.4. After a 1 h incubation, plates are washed and 3-fold serial dilution of antibodies in PBS containing 0.5% BSA, 0.05% polysorbate 20, 15 ppm Proclin, pH 7.4 is added to the plates and incubated for 2 h. For enhanced binding sensitivity due to avidity, immune complexes are formed using anti-human antibody. Bound antibody is detected with HRP-conjugated goat anti-human kappa (Southern Biotech) using Ultra TMB substrate as described in Example 1. The reaction is terminated and the plate is read as described above. The dose-dependent binding curve of the wild type antibody (no Fc modifications) is fitted with GraphPad Prism (Sunnyvale, CA) four parameter curve fitting program. The relative affinity of the variant vs. the wild type is estimated by dividing the equivalent ng/ml wild type concentration at the appropriate concentration.

In addition, the variants are tested directly in Fc effector bioassays (Promega) following manufacturer's directions. These assays include FcγRIIa-H ADCP Bioassay (Promega cat #09901), ADCC Reporter Bioassays, FcγRIIIa F Variant Promeg: cal #G9798), ADCC Reporter Bioassays, FcγRIlla V Variant (Promega, cat. #G7015). The variants are tested both as monomeric lg and as small immune complexes (ICs) by using an anti-hu Ig antibody to form small ICs.

A Europium based ADCC assay is performed. Briefly, peripheral blood lymphocytes (PBLs) are isolated by Ficoll Paque Plus gradient centrifugation. The PBLs are collected, washed with RPMI1640, 10% FCS and resuspended in cell culture medium. The cells are diluted to 2.5×10⁶ cells/ml. Target cells are labelled with BADTA (2,2′: 6′, 2″-terpyridine-6,6″-dicarboxylic acid acetoxymethylester): Cells are harvested by adding Accutase (Millipore), washed once and diluted to 1×10⁶ cells/ml. Next, 2.5 μL BADTA is added per 1×10⁶ cells and incubated for 35 min at 37° C. with 5% CO₂. After labelling the cells are diluted with 10 ml culture medium, centrifuged at 200×g for 10 min and supernatant aspirated. This step is repeated 3× with culture medium/2 mM Probenicid and the sample is diluted to 1×10⁵ cells/ml, centrifuged at 300×g for 5 min, supernatant taken off and 50 μL pipetted into the wells intended for the background controls. The final ratio of effector (PBL) to target cells is 25:1.

Controls include: (1) Background: the 50 μL aliquot, diluted with 100 μL medium, (2) Spontaneous lysis: 50 μL of the labelled target cell suspension plus 100 μL culture medium, incubated 2 h at 37° C., (3) Maximal lysis: 50 μL/well of the labelled target cell suspension plus 100 μL Triton X-100 (0.5% in PBS) incubated 2 h at 37° C., (4) Lysis control without antibodies: 50 μL/well of the labelled target cell suspension and 50 μL culture medium plus 50 μL of effector cells incubated 2 h at 37° C., (5) Lysis control without effector cells: 50 μL/well of the labelled target cell suspension; add 50 μL culture medium plus antibody at highest concentration used and incubate 2 h at 37° C.

At the end of the incubation period the 96 well plate is centrifuged at 100 rpm. 20 μL of each supernatant is transferred into an OptiPlate HTRF-96 (Packard) and 200 μL Europium solution is added and incubated for 15 min on a shaker. Fluorescence is measured as for time resolved fluorescence and spontaneous release and specific release are calculated.

A CDC assay is performed. Briefly, target cells are washed and diluted to 1×10⁵ cells/ml and 100 μL/well (10⁴ cells) are added to a 96-well flat bottom microtiter plate. A titration curve of the test antibody is created using serial dilutions, beginning at 1 μg/mL. Antibody is added to the plate, mixed gently, and is then placed at 37° C./5% CO₂ incubator for 30 min. Next, 25 μL freshly dissolved baby rabbit complement (Cedarlane CL3441, 1 ml lyophilized, dilute freshly in 4 ml double distilled water) is added, mixed gently, and the plate is incubated at 37° C./5% CO₂ incubator for 30 min. After the incubation period 50 μL supernatant is taken off and 100 μL Cell Titer Glo. reagent (Promega Corp.) is added to the remaining 100 μL supernatant. The plate is placed on an orbital shaker for 2 min, 100 μL/well is transferred into a black luminescence microtiter plate (Costar) and luminescence is measured.

Controls included: (1) medium control (target cells plus 50 μL medium), (2) maximal lysis control (target cells plus 50 μL 0.5% Triton X-100), (3) complement control (target cells plus 25 uL medium plus 25 μl complement).

### 7.4 Example 4: Characterization of Potency and Species Selectivity in Whole Blood Assay

The relative potency of a panel of candidate antibodies was first assessed by determining the inhibition of interferon gamma release in human blood using the antibodies at 1 and 10 nM. All of the antibodies displayed potent activity, with A219 appearing to be one of the most potent candidates (Table 13).

TABLE 13

| Clone | % Inhibition at 1 nM Ig | % Inhibition at 10 nM Ig |
|---|---|---|
| A147 | 51.3 | 72.4 |
| A212 | 46.8 | 71.2 |
| A213 | 48.6 | 69.8 |
| A217 | 46.0 | 72.2 |
| A219 | 59.8 | 75.2 |
| A220 | 36.9 | 63.2 |

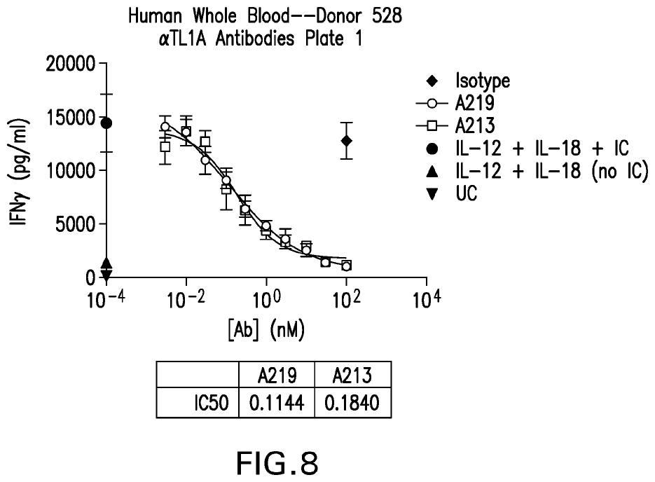

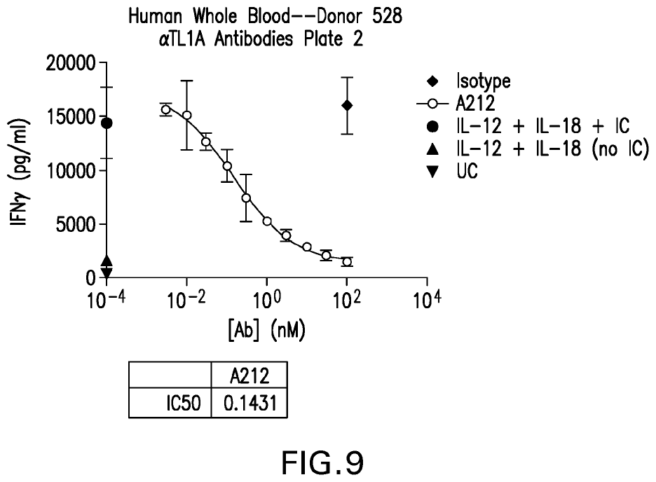

Next, three of the variants were characterized for inhibition of interferon gamma release in human blood using multiple human blood donors and testing the antibodies across a broader range of concentrations (0.01-100 nM). Representative inhibition profiles of variants A212, A213 and A219 are shown in FIGS. 8-9. The mean IC50 values for these variants, and a control antibody termed ID1, for the inhibition of interferon gamma release from multiple human donors is shown in Table 14.

TABLE 14

| Clone | Mean | SD |
|---|---|---|
| A212 | 51.3 | 72.4 |
| A213 | 46.8 | 71.2 |
| A219 | 48.6 | 69.8 |
| 1D1 | 46.0 | 72.2 |

### 7.5 Example 5: In Vivo Assessment Of Anti-TL1a Efficacy

The efficacy of anti-TL1A antibodies in animal models of colitis is performed. Anti-TL1A antibodies are tested in rodent models of acute colitis induced by intrarectal administration of di- or tri-nitrobenzenesulfonic acid (D/TNBS) or oxazolone, and chronic colitis induced by administration of DSS in drinking water or transfer of CD4SRB^{hi} T cells. DNBS and oxazolone induce localized ulceration and inflammation. DSS administration induces robust generalized inflammation of the intestinal tract characterized by erosive lesions and inflammatory infiltrate. Symptoms of all these models usually include diarrhea, occult blood, weight loss and occasionally rectal prolapse. In a prophylactic model, antibody treatment begins at the start of administration of the colitis-inducing compound. In a therapeutic model, antibody treatment begins several days after commencement of induction. The effect of the treatment on weight, stool consistency and occult blood, as well as microscopic effects on epithelial integrity and degree of inflammatory infiltrate is determined. Daily clinical scoring is performed based on stool consistency and presence of occult blood giving a disease activity index (DAI) score.

TABLE 15
CDR Amino Acid Sequences

| SEQ | | |
|---|---|---|
| ID | | |
| NO | Description | Sequence |
| 1 | P HCDR1 | GFDIQDTYMH |
| 601 | P HCDR1 | DTYMH |
| 602 | P HCDR1 | KYDIN |
| 603 | P HCDR1 | GFEIQDTYMH |
| 604 | P HCDR1 | GFDPQDTYMH |
| 605 | P HCDR1 | GFDVQDTYMH |
| 606 | P HCDR1 | GFDIGDTYMH |
| 607 | P HCDR1 | GFDISDTYMH |
| 608 | P HCDR1 | GFDIVDTYMH |
| 609 | P HCDR1 | GFDIQDAYMH |
| 610 | P HCDR1 | GFDIQDSYMH |
| 611 | P HCDR1 | GFDIQDTFMH |
| 612 | P HCDR1 | GFDIQDTYIH |
| 613 | P HCDR1 | GFDLQDTYMH |
| 614 | P HCDR1 | GFDIQDTYLH |
| 615 | P HCDR1 | GFDIGDTFIH |
| 616 | P HCDR1 | GFDIGDTFMH |
| 617 | P HCDR1 | GFDIGDTYIH |
| 618 | P HCDR1 | GFDIQDTFIH |
| 619 | P HCDR1 | GFDIQDTFMH |
| 620 | P HCDR1 | GFDIQDTYIH |
| 621 | P HCDR1 | GFDISDTFIH |
| 622 | P HCDR1 | GFDISDTFMH |
| 623 | P HCDR1 | GFDISDTYIH |
| 624 | P HCDR1 | GFDISDTYMH |
| 625 | P HCDR1 | GFDIVDTFIH |
| 626 | P HCDR1 | GFDIVDTFMH |
| 627 | P HCDR1 | GFDIVDTYIH |
| 628 | P HCDR1 | GFDPGDTFIH |
| 629 | P HCDR1 | GFDPGDTFMH |
| 630 | P HCDR1 | GFDPGDTYIH |
| 631 | P HCDR1 | GFDPGDTYMH |
| 632 | P HCDR1 | GFDPQDTFIH |
| 633 | P HCDR1 | GFDPQDTFMH |
| 634 | P HCDR1 | GFDPQDTYTH |
| 635 | P HCDR1 | GFDPQDTYMH |
| 636 | P HCDR1 | GFDPSDTFIH |
| 637 | P HCDR1 | GFDPSDTFMH |
| 638 | P HCDR1 | GFDPSDTYIH |
| 639 | P HCDR1 | GFDPSDTYMH |
| 640 | P HCDR1 | GFDPVDTFIH |
| 641 | P HCDR1 | GFDPVDTFMH |
| 642 | P HCDR1 | GFDPVDTYIH |
| 643 | P HCDR1 | GFDPVDTYMH |
| 644 | P HCDR1 | GFDVGDTFIH |
| 645 | P HCDR1 | GFDVGDTFMH |
| 646 | P HCDR1 | GFDVGDTYIH |
| 647 | P HCDR1 | GFDVGDTYMH |
| 648 | P HCDR1 | GFDVQDTFIH |
| 649 | P HCDR1 | GFDVQDTFMH |
| 650 | P HCDR1 | GFDVQDTYIH |
| 651 | P HCDR1 | GFDVSDTFIH |
| 652 | P HCDR1 | GFDVSDTFMH |
| 653 | P HCDR1 | GFDVSDTYIH |
| 654 | P HCDR1 | GFDVSDTYMH |
| 655 | P HCDR1 | GFDVVDTFIH |
| 656 | P HCDR1 | GFDVVDTFMH |
| 657 | P HCDR1 | GFDVVDTYIH |
| 658 | P HCDR1 | GFDVVDTYMH |
| 659 | P HCDR1 | GFEIGDTFIH |
| 660 | P HCDR1 | GFEIGDTFMH |
| 661 | P HCDR1 | GFEIGDTYIH |
| 662 | P HCDR1 | GFEIGDTYMH |
| 663 | P HCDR1 | GFEIQDTFIH |
| 664 | P HCDR1 | GFEIQDTFMH |
| 665 | P HCDR1 | GFEIQDTYIH |
| 666 | P HCDR1 | GFEIQDTYMH |
| 667 | P HCDR1 | GFEISDTFIH |
| 668 | P HCDR1 | GFEISDTFMH |
| 669 | P HCDR1 | GFEISDTYIH |
| 670 | P HCDR1 | GFEISDTYMH |
| 671 | P HCDR1 | GFEIVDTFIH |
| 672 | P HCDR1 | GFEIVDTFMH |
| 673 | P HCDR1 | GFEIVDTYIH |
| 674 | P HCDR1 | GFEIVDTYMH |
| 675 | P HCDR1 | GFEPGDTFIH |
| 676 | P HCDR1 | GFEPGDTFMH |
| 677 | P HCDR1 | GFEPGDTYIH |
| 678 | P HCDR1 | GFEPGDTYMH |
| 679 | P HCDR1 | GFEPQDTFIH |
| 680 | P HCDR1 | GFEPQDTFMH |
| 681 | P HCDR1 | GFEPQDTYIH |
| 682 | P HCDR1 | GFEPQDTYMH |
| 683 | P HCDR1 | GFEPSDTFIH |
| 684 | P HCDR1 | GFEPSDTFMH |
| 685 | P HCDR1 | GFEPSDTYIH |
| 686 | P HCDR1 | GFEPSDTYMH |
| 687 | P HCDR1 | GFEPVDTFIH |
| 688 | P HCDR1 | GFEPVDTFMH |
| 689 | P HCDR1 | GFEPVDTYIH |
| 690 | P HCDR1 | GFEPVDTYMH |
| 691 | P HCDR1 | GFEVGDTFIH |
| 692 | P HCDR1 | GFEVGDTFMH |
| 693 | P HCDR1 | GFEVGDTYIH |
| 694 | P HCDR1 | GFEVGDTYMH |
| 695 | P HCDR1 | GFEVQDTFIH |
| 696 | P HCDR1 | GFEVQDTFMH |
| 697 | P HCDR1 | GFEVQDTYIH |
| 698 | P HCDR1 | GFEVQDTYMH |
| 699 | P HCDR1 | GFEVSDTFIH |
| 700 | P HCDR1 | GFEVSDTFMH |
| 701 | P HCDR1 | GFEVSDTYIH |
| 702 | P HCDR1 | GFEVSDTYMH |
| 703 | P HCDR1 | GFEVVDTFIH |
| 704 | P HCDR1 | GFEVVDTFMH |
| 705 | P HCDR1 | GFEVVDTYIH |
| 706 | P HCDR1 | GFEVVDTYMH |
| 707 | P HCDR1 | GFX₁X₂X₃DX₄X₅X₆H |
| | | X₁ = D or E |
| | | X₂ = I, L, P, or V |
| | | X₃ = G, Q, S, or V |
| | | X₄ = A, S, T |
| | | X₅ = F or Y |
| | | X₆ = I, L, or M |
| 708 | P HCDR1 | GFX₁X₂X₃DTX₄X₅H |
| | | X₁ = D, OR E |
| | | X₂ = I, P, OR V |
| | | X₃ = G, Q, S, OR V |
| | | X₄ = F, OR Y |
| | | X₅ = I, OR M |
| 709 | M1 HCDR1 | GFTFSSYW |
| 710 | M2 HCDR1 | GGSFTGFY |
| 711 | M3 HCDR1 | GY(X1)F(X2)(X3)YGIS; X1 = P, |
| | | S, D, Q, N; X2 = T, R; |
| | | X3 = N, T, Y, H |
| 712 | M3 HCDR1 | GYDFTYYGIS |
| 13 | M4 HCDR1 | SRSYYWG |
| 714 | MS HCDR1 | TSNMGVV |
| 715 | M6 HCDR1 | LYGMN |
| 716 | M6 HCDR1 | NYGMN |
| 717 | M7 HCDR1 | GYTFTSSWMH |
| 18 | M8 HCDR1 | GYTFTSYDIN |
| 719 | M9 HCDR1 | SYFWS |
| 720 | M10 HCDR1 | GYYWN |
| 721 | M11 HCDR1 | GFTFSTYG |
| 722 | M12 HCDR1 | TYGMS |
| 2 | P HCDR2 | RIDPASGHTKYDPKFQV |
| 3 | P HCDR2 | RIEPASGHIKYDPKFQG |
| 1 | P HCDR2 | RIDPASGHIKYDPKFQG |
| 5 | P HCDR2 | RIEPASGHIKYDPKFQV |
| 723 | P HCDR2 | WIFPGDGRTDYNEKFKG |
| 724 | P HCDR2 | RLDPASGHTK |
| 725 | P HCDR2 | RIEPASGHTK |
| 726 | P HCDR2 | RIDPESGHTK |
| 727 | P HCDR2 | RIDPASGHTK |
| 728 | P HCDR2 | RIDPAGGHTK |
| 729 | P HCDR2 | RIDPASAHTK |
| 730 | P HCDR2 | RIDPASGHIK |
| 731 | P HCDR2 | RIDPASGHLK |
| 732 | P HCDR2 | RIDPASGHVK |
| 733 | P HCDR2 | YDPKFQV |
| 734 | P HCDR2 | IDPKFQV |
| 735 | P HCDR2 | LDPKFQV |
| 736 | P HCDR2 | MDPKFQV |
| 737 | P HCDR2 | SDPKFQV |
| 738 | P HCDR2 | TDPKFQV |
| 739 | P HCDR2 | VDPKFQV |
| 740 | P HCDR2 | YIPKFQV |
| 741 | P HCDR2 | YNPKFQV |
| 742 | P HCDR2 | YRPKFQV |
| 743 | P HCDR2 | YSPKFQV |
| 744 | P HCDR2 | YDPKFRV |
| 745 | P HCDR2 | YDPKFQA |
| 746 | P HCDR2 | YDPKFQD |
| 747 | P HCDR2 | YDPKFQE |
| 748 | P HCDR2 | YDPKFQG |
| 749 | P HCDR2 | YDPKFQH |
| 750 | P HCDR2 | YDPKFQK |
| 751 | P HCDR2 | YDPKFQL |
| 752 | P HCDR2 | YDPKFQM |
| 753 | P HCDR2 | YDPKFQN |
| 754 | P HCDR2 | YDPKFQP |
| 755 | P HCDR2 | YDPKFQR |
| 756 | P HCDR2 | YDPKFQS |
| 757 | P HCDR2 | YDPKFQT |
| 758 | P HCDR2 | RIEPASGHIKYDPKFQG |
| 759 | P HCDR2 | RIEPASGHIKYSPKFQG |
| 760 | P HCDR2 | RIEPASGHVKYSPKFQV |
| 761 | P HCDR2 | RIEPASGHVKYDPKFQT |
| 762 | P HCDR2 | RIDPASGHIKYDPKFQK |
| 763 | P HCDR2 | RIDPASGHVKIDPKFQV |
| 764 | P HCDR2 | RIDPASGHLKYDPKFQV |
| 765 | P HCDR2 | RIDPASGHLKYDPKFQR |
| 766 | P HCDR2 | RIDPASGHLKYDPKFQN |
| 767 | P HCDR2 | RIEPASGHLKYDPKFQE |
| 768 | P HCDR2 | PASGH |
| 769 | P HCDR2 | RIDPASGHTKYDPKFQV |
| 770 | P HCDR2 | RIDPASGHLKYDPKFQG |
| 771 | P HCDR2 | RIDPASGHTKYDPKFQG |
| 772 | P HCDR2 | RIDPASGHSKYDPKFQV |
| 773 | P HCDR2 | RIDPASGHYKYDPKFQV |
| 774 | P HCDR2 | RX₁X₂PX₃X₄X₅HX₆KX₇X₈PKFX₉X₁₀ |
| | | X₁ = I or L |
| | | X₂ = D or E |
| | | X₃ = A or E |
| | | X₄ = G or S |
| | | X₅ = A or G |
| | | X₆ = I, L, T, or V |
| | | X₇ = I, L, M, S, T, V, or Y |
| | | X₈ = D, I, N, R, or S |
| | | X₉ = Q or R |
| | | X₁₀ = A, D, E, G, H, K, L, |
| | | M, N, P, R, S, T, or V |
| 775 | M1 HCDR2 | IKEDGSEK |
| 776 | M2 HCDR2 | INHRGNT |
| 777 | M3 HCDR2 | WIS(X1)YNG(X2)(X3)(X4)YA(X5) |
| | | (X6)(X7)QG; X1 = T, P, S, |
| | | A; X2 = N, G, V. K. A; |
| | | X3 = T, K; X4 = H, N; X5 = |
| | | Q, R; X6 = K, M; X7 = L, H |
| 778 | M3 HCDR2 | WISTYNGNTHYARMLQG |
| 779 | M4 HCDR2 | SIYYNGRTYYNPSLKS |
| 780 | M5 HCDR2 | HILWDDREYSNPALKS |
| 781 | M6 HCDR2 | WINTYTGEPTYADDFKG |
| 782 | M7 HCDR2 | IHPNSGGT |
| 783 | M8 HCDR2 | WLNPNSGXTG; X = N, Y |
| 784 | M9 HCDR2 | YIYYSGNTKYNPSLKS |
| 785 | M10 HCDR2 | EINHAGNTNYNPSLKS |
| 786 | M11 HCDR2 | ISGTGRTT |
| 787 | M12 HCDR2 | WMNTYSGVTTYADDFKG |
| 6 | P HCDR3 | SGGLPDV |
| 7 | P HCDR3 | ARSGGLPDV |
| 8 | P HCDR3 | SGGLPDW |
| 9 | P HCDR3 | ARSGGLPDW |
| 788 | P HCDR3 | YGPAMDY |
| 789 | P HCDR3 | LGGLPDV |
| 790 | P HCDR3 | SAGLPDV |
| 791 | P HCDR3 | SGGAPDV |
| 792 | P HCDR3 | SGGMPDV |
| 793 | P HCDR3 | SGGLPEV |
| 794 | P HCDR3 | SGGLPDK |
| 795 | P HCDR3 | SGGLPDM |
| 796 | P HCDR3 | SGGLPDQ |
| 797 | P HCDR3 | SGGLPDR |
| 798 | P HCDR3 | SGGLPDS |
| 799 | P HCDR3 | SGGLPDT |
| 800 | P HCDR3 | SGGLPDH |
| 801 | P HCDR3 | SGGLPDF |
| 802 | P HCDR3 | SGGSPDV |
| 803 | P HCDR3 | ARSGGLPDM |
| 804 | P HCDR3 | ARSGGLPDK |
| 805 | P HCDR3 | SGGLPD |
| 806 | P HCDR3 | ARSGGLPDF |
| 807 | P HCDR3 | ARSGGLPDL |
| 808 | P HCDR3 | SGGLPDE |
| 809 | P HCDR3 | SGGLPDI |
| 810 | P HCDR3 | SGGLPDK |
| 811 | P HCDR3 | SGGLPDL |
| 812 | P HCDR3 | SGGLPDM |
| 813 | P HCDR3 | SGGLPDQ |
| 814 | P HCDR3 | SGGLPDT |
| 815 | P HCDR3 | SGGLPDW |
| 816 | P HCDR3 | SGGLPDY |
| 817 | P HCDR3 | SGGMPDE |
| 818 | P HCDR3 | SGGMPDI |
| 819 | P HCDR3 | SGGMPDK |
| 820 | P HCDR3 | SGGMPDL |
| 821 | P HCDR3 | SGGMPDM |
| 822 | P HCDR3 | SGGMPDQ |
| 823 | P HCDR3 | SGGMPDT |
| 824 | P HCDR3 | SGGMPDV |
| 825 | P HCDR3 | SGGMPDW |
| 826 | P HCDR3 | SGGMPDY |
| 827 | P HCDR3 | X₁X₂GX₃PX₄X₅ |
| | | X₁ = L or S |
| | | X₂ = A or G |
| | | X₃ = A, L, or M |
| | | X₄ = D or B |
| | | X₅ = K, M, Q, R, S, T, V, |
| | | or W |
| 828 | P HCDR3 | SGGX₁PDX₂ |
| | | X₁ = L, OR M |
| | | X₂ = E, I, K, L, M, Q, T, V, |
| | | W, OR Y |
| 829 | M1 HCDR3 | AREDYDSYYKYGMDV |
| 830 | M2 HCDR3 | ASPFYDFWSGSDY |
| 831 | M3 HCDR3 | ENYYGSG(X1)(X2)R GGMD(X3); |
| | | X1 = S, A; X2 = Y, P; |
| | | X3 = V, A, G |
| 832 | M3 HCDR3 | ENYYGSGAYRGGMDV |
| 833 | M4 HCDR3 | EDYGDYGAFDI |
| 834 | M5 HCDR3 | MSRNYYGSSYVMDY |
| 835 | M6 HCDR3 | DTAMDYAMAY |
| 836 | M6 HCDR3 | DYGKYGDYYAMDY |
| 837 | M7 HCDR3 | ARGDYYGYVSWFAY |
| 838 | M8 HCDR3 | EVPETAAFEY |
| 839 | M9 HCDR3 | ETGSYYGFDY |
| 840 | M10 HCDR3 | GYCRSTTCYFDY |
| 841 | M11 HCDR3 | TKERGDYYYGVFDY |
| 842 | M12 HCDR3 | EGYVEDDYYATDY |
| 10 | P LCDR1 | RASSSVSYMY |
| 843 | P LCDR1 | RSSQTIVHSNGDTYLD |
| 844 | P LCDR1 | GASSSVSYMY |
| 845 | P LCDR1 | WASSSVSYMY |
| 846 | P LCDR1 | RASSSVIYMY |
| 847 | P LCDR1 | RASSSVSFMY |
| 848 | P LCDR1 | RASSSVSYLY |
| 849 | P LCDR1 | RASSSVSYMR |
| 850 | P LCDR1 | GASSSVSYMY |
| 851 | P LCDR1 | ASSSVSYMY |
| 852 | P LCDR1 | X₁ASSSVX₂X₃X₄X₅ |
| | | X₁ = G, R, or W |
| | | X₂ = I or S |
| | | X₃ = F or Y |
| | | X₄ = L or M |
| | | X₅ = R or Y |
| 853 | M1 LCDR1 | QSILYSSNNKNY |
| 854 | M2 LCDR1 | QSLVHSDGNTY |
| 855 | M3 LCDR1 | RASQSVSSYLA |
| 856 | M4 LCDR1 | RASQGISSALA |
| 857 | M5 LCDR1 | SASSSVNYMH |
| 858 | M6 LCDR1 | KSSQNIVHSDGNTYLE |
| 859 | M6 LCDR1 | RSSQSIVHSNGNTYLD |
| 860 | M7 LCDR1 | QNINVL |
| 861 | M8 LCDR1 | TSSSSDIGA(X1)(X2)GV(X3); |
| | | X1 = G, A; X2 = L, S, Q; |
| | | X3 = H, L |
| 862 | M9 LCDR1 | RASQSINNYLN |
| 863 | M10 LCDR1 | RASQSVRSSYLA |
| 864 | M11 LCDR1 | QTISSW |
| 865 | M12 LCDR1 | RSSQNIVHSDGNTYLE |
| 11 | P LCDR2 | ATSNLAS |
| 866 | P LCDR2 | KVSNRFS |
| 867 | P LCDR2 | AKSNLAS |
| 868 | P LCDR2 | ATPNLAS |
| 869 | P LCDR2 | ATENLAS |
| 870 | P LCDR2 | ATSLLAS |
| 871 | P LCDR2 | ATSPLAS |
| 872 | P LCDR2 | ATSNLTS |
| 873 | P LCDR2 | AX₁X₂X₃LX₄S |
| | | X1 = K or T |
| | | X2 = E, P, or S |
| | | X3 = L, N, or P |
| | | X4 = A or T |
| 874 | M1 LCDR2 | WAS |
| 875 | M2 LCDR2 | KIS |
| 876 | M3 LCDR2 | DASNRAT |
| 877 | M4 LCDR2 | DASSLES |
| 878 | MS LCDR2 | STSNLAS |
| 879 | M6 LCDR2 | KVSNRFS |
| 880 | M7 LCDR2 | KAS |
| 881 | M8 LCDR2 | GYYNRPS |
| 882 | M9 LCDR2 | AASSLQS |
| 883 | M10 LCDR2 | GASSRAT |
| 884 | M11 LCDR2 | AAS |
| 885 | M12 LCDR2 | KVSNRFS |
| 12 | P LCDR3 | QQWEGNPRT |
| 13 | P LCDR3 | QQWKGNPRT |
| 14 | P LCDR3 | QQWSGNPRT |
| 15 | P LCDR3 | QQWSRNPRT |
| 886 | P LCDR3 | FQGSHVPYT |
| 887 | P LCDR3 | HQWSGNPRT |
| 888 | P LCDR3 | NQWSGNPRT |
| 889 | P LCDR3 | SQWSGNPRT |
| 890 | P LCDR3 | QQSSGNPRT |
| 891 | P LCDR3 | QQWDGNPRT |
| 892 | P LCDR3 | QQWHGNPRT |
| 893 | P LCDR3 | QQWNGNPRT |
| 894 | P LCDR3 | QQWQGNPRT |
| 895 | P LCDR3 | QQWVGNPRT |
| 896 | P LCDR3 | QQWSANPRT |
| 897 | P LCDR3 | QQWSDNPRT |
| 898 | P LCDR3 | QQWSQNPRT |
| 899 | P LCDR3 | QQWSSNPRT |
| 900 | P LCDR3 | QQWSGNPRS |
| 901 | P LCDR3 | QQFSGNPRT |
| 902 | P LCDR3 | QQHSGNPRT |
| 903 | P LCDR3 | QQISGNPRT |
| 904 | P LCDR3 | QQPSGNPRT |
| 905 | P LCDR3 | QQRSGNPRT |
| 906 | P LCDR3 | QQYSGNPRT |
| 907 | P LCDR3 | QQWSGHPRT |
| 908 | P LCDR3 | QQWSGLPRT |
| 909 | P LCDR3 | QQWSGQPRT |
| 910 | P LCDR3 | QQWSGSPRT |
| 911 | P LCDR3 | QQWSGTPRT |
| 912 | P LCDR3 | QQWSGMPRT |
| 913 | P LCDR3 | QQWSGFPRT |
| 914 | P LCDR3 | QQWSGKPRT |
| 915 | P LCDR3 | QQWSGRPRT |
| 916 | P LCDR3 | QQWSGDPRT |
| 917 | P LCDR3 | QQWAGNPRT |
| 918 | P LCDR3 | QQWYGNPRT |
| 919 | P LCDR3 | QQWFGNPRT |
| 920 | P LCDR3 | QQWQGNPRT |
| 921 | P LCDR3 | GNPRT |
| 922 | P LCDR3 | SQWSGNPRT |
| 923 | P LCDR3 | QQWSGNPRS |
| 924 | P LCDR3 | QQWSGTPRT |
| 925 | P LCDR3 | QQWSGDPRT |
| 926 | P LCDR3 | QQWSGFPRT |
| 927 | P LCDR3 | QQWSGKPRT |
| 928 | P LCDR3 | QQWSGRPRT |
| 929 | P LCDR3 | QQWSGSPRT |
| 930 | P LCDR3 | QQWDADPRT |
| 931 | P LCDR3 | QQWDAFPRT |
| 932 | P LCDR3 | QQWDAKPRT |
| 933 | P LCDR3 | QQWDANPRT |
| 934 | P LCDR3 | QQWDARPRT |
| 935 | P LCDR3 | QQWDASPRT |
| 936 | P LCDR3 | QQWDATPRT |
| 937 | P LCDR3 | QQWDGDPRT |
| 938 | P LCDR3 | QQWDGFPRT |
| 939 | P LCDR3 | QQWDGKPRT |
| 940 | P LCDR3 | QQWDGNPRT |
| 941 | P LCDR3 | QQWDGRPRT |
| 942 | P LCDR3 | QQWDGSPRT |
| 943 | P LCDR3 | QQWDGTPRT |
| 944 | P LCDR3 | QQWEADPRT |
| 945 | P LCDR3 | QQWEAFPRT |
| 946 | P LCDR3 | QQWEAKPRT |
| 947 | P LCDR3 | QQWEANPRT |
| 948 | P LCDR3 | QQWEARPRT |
| 949 | P LCDR3 | QQWEASPRT |
| 950 | P LCDR3 | QQWEATPRT |
| 951 | P LCDR3 | QQWEGDPRT |
| 952 | P LCDR3 | QQWEGFPRT |
| 953 | P LCDR3 | QQWEGKPRT |
| 954 | P LCDR3 | QQWEGRPRT |
| 955 | P LCDR3 | QQWEGSPRT |
| 956 | P LCDR3 | QQWEGTPRT |
| 957 | P LCDR3 | QQWHADPRT |
| 958 | P LCDR3 | QQWHAFPRT |
| 959 | P LCDR3 | QQWHAKPRT |
| 960 | P LCDR3 | QQWHANPRT |
| 961 | P LCDR3 | QQWHARPRT |
| 962 | P LCDR3 | QQWHASPRT |
| 963 | P LCDR3 | QQWHATPRT |
| 964 | P LCDR3 | QQWHGDPRT |
| 965 | P LCDR3 | QQWHGFPRT |
| 966 | P LCDR3 | QQWHGKPRT |
| 967 | P LCDR3 | QQWHGNPRT |
| 968 | P LCDR3 | QQWHGRPRT |
| 969 | P LCDR3 | QQWHGSPRT |
| 970 | P LCDR3 | QQWHGTPRT |
| 971 | P LCDR3 | QQWNADPRT |
| 972 | P LCDR3 | QQWNAFPRT |
| 973 | P LCDR3 | QQWNAKPRT |
| 974 | P LCDR3 | QQWNANPRT |
| 975 | P LCDR3 | QQWNARPRT |
| 976 | P LCDR3 | QQWNASPRT |
| 977 | P LCDR3 | QQWNATPRT |
| 978 | P LCDR3 | QQWNGDPRT |
| 979 | P LCDR3 | QQWNGFPRT |
| 980 | P LCDR3 | QQWNGKPRT |
| 981 | P LCDR3 | QQWNGNPRT |
| 982 | P LCDR3 | QQWNGRPRT |
| 983 | P LCDR3 | QQWNGSPRT |
| 984 | P LCDR3 | QQWNGTPRT |
| 985 | P LCDR3 | QQWQADPRT |
| 986 | P LCDR3 | QQWQAFPRT |
| 987 | P LCDR3 | QQWQAKPRT |
| 988 | P LCDR3 | QQWQANPRT |
| 989 | P LCDR3 | QQWQARPRT |
| 990 | P LCDR3 | QQWQASPRT |
| 991 | P LCDR3 | QQWQATPRT |
| 992 | P LCDR3 | QQWQGDPRT |
| 993 | P LCDR3 | QQWQGFPRT |
| 994 | P LCDR3 | QQWQGKPRT |
| 995 | P LCDR3 | QQWQGRPRT |
| 996 | P LCDR3 | QQWQGSPRT |
| 997 | P LCDR3 | QQWQGTPRT |
| 998 | P LCDR3 | QQWSADPRT |
| 999 | P LCDR3 | QQWSAFPRT |
| 1000 | P LCDR3 | QQWSAKPRT |
| 1001 | P LCDR3 | QQWSANPRT |
| 1002 | P LCDR3 | QQWSARPRT |
| 1003 | P LCDR3 | QQWSASPRT |
| 1004 | P LCDR3 | QQWSATPRT |
| 1005 | P LCDR3 | NQWDAFPRT |
| 1006 | P LCDR3 | NQWDAKPRT |
| 1007 | P LCDR3 | NQWDANPRT |
| 1008 | P LCDR3 | NQWDARPRT |
| 1009 | P LCDR3 | NQWDASPRT |
| 1010 | P LCDR3 | NQWDATPRT |
| 1011 | P LCDR3 | NQWDGDPRT |
| 1012 | P LCDR3 | NQWDGFPRT |
| 1013 | P LCDR3 | NQWDGKPRT |
| 1014 | P LCDR3 | NQWDGNPRT |
| 1015 | P LCDR3 | NQWDGRPRT |
| 1016 | P LCDR3 | NQWDGSPRT |
| 1017 | P LCDR3 | NQWDGTPRT |
| 1018 | P LCDR3 | NQWEADPRT |
| 1019 | P LCDR3 | NQWEAFPRT |
| 1020 | P LCDR3 | NQWEAKPRT |
| 1021 | P LCDR3 | NQWEANPRT |
| 1022 | P LCDR3 | NQWEARPRT |
| 1023 | P LCDR3 | NQWEASPRT |
| 1024 | P LCDR3 | NQWEATPRT |
| 1025 | P LCDR3 | NQWEGDPRT |
| 1026 | P LCDR3 | NQWEGFPRT |
| 1027 | P LCDR3 | NQWEGKPRT |
| 1028 | P LCDR3 | NQWEGNPRT |
| 1029 | P LCDR3 | NQWEGRPRT |
| 1030 | P LCDR3 | NQWEGSPRT |
| 1031 | P LCDR3 | NQWEGTPRT |
| 1032 | P LCDR3 | NQWHADPRT |
| 1033 | P LCDR3 | NQWHAFPRT |
| 1034 | P LCDR3 | NQWHAKPRT |
| 1035 | P LCDR3 | NQWHANPRT |
| 1036 | P LCDR3 | NQWHARPRT |
| 1037 | P LCDR3 | NQWHASPRT |
| 1038 | P LCDR3 | NQWHATPRT |
| 1039 | P LCDR3 | NQWHGDPRT |
| 1040 | P LCDR3 | NQWHGFPRT |
| 1041 | P LCDR3 | NQWHGKPRT |
| 1042 | P LCDR3 | NQWHGNPRT |
| 1043 | P LCDR3 | NQWHGRPRT |
| 1044 | P LCDR3 | NQWHGSPRT |
| 1045 | P LCDR3 | NQWHGTPRT |
| 1046 | P LCDR3 | NQWNADPRT |
| 1047 | P LCDR3 | NQWNAFPRT |
| 1048 | P LCDR3 | NQWNAKPRT |
| 1049 | P LCDR3 | NQWNANPRT |
| 1050 | P LCDR3 | NQWNARPRT |
| 1051 | P LCDR3 | NQWNASPRT |
| 1052 | P LCDR3 | NQWNATPRT |
| 1053 | P LCDR3 | NQWNGDPRT |
| 1054 | P LCDR3 | NQWNGFPRT |
| 1055 | P LCDR3 | NQWNGKPRT |
| 1056 | P LCDR3 | NQWNGNPRT |
| 1057 | P LCDR3 | NQWNGRPRT |
| 1058 | P LCDR3 | NQWNGSPRT |
| 1059 | P LCDR3 | NQWNGTPRT |
| 1060 | P LCDR3 | NQWQADPRT |
| 1061 | P LCDR3 | NQWQAFPRT |
| 1062 | P LCDR3 | NQWQAKPRT |
| 1063 | P LCDR3 | NQWQANPRT |
| 1064 | P LCDR3 | NQWQARPRT |
| 1065 | P LCDR3 | NQWQASPRT |
| 1066 | P LCDR3 | NQWQATPRT |
| 1067 | P LCDR3 | NQWQGDPRT |
| 1068 | P LCDR3 | NQWQGFPRT |
| 1069 | P LCDR3 | NQWQGKPRT |
| 1070 | P LCDR3 | NQWQGNPRT |
| 1071 | P LCDR3 | NQWQGRPRT |
| 1072 | P LCDR3 | NQWQGSPRT |
| 1073 | P LCDR3 | NQWQGTPRT |
| 1074 | P LCDR3 | NQWSADPRT |
| 1075 | P LCDR3 | NQWSAFPRT |
| 1076 | P LCDR3 | NQWSAKPRT |
| 1077 | P LCDR3 | NQWSANPRT |
| 1078 | P LCDR3 | NQWSARPRT |
| 1079 | P LCDR3 | NQWSASPRT |
| 1080 | P LCDR3 | NQWSATPRT |
| 1081 | P LCDR3 | NQWSGDPRT |
| 1082 | P LCDR3 | NQWSGFPRT |
| 1083 | P LCDR3 | NQWSGKPRT |
| 1084 | P LCDR3 | NQWSGNPRT |
| 1085 | P LCDR3 | NQWSGRPRT |
| 1086 | P LCDR3 | NQWSGSPRT |
| 1087 | P LCDR3 | NQWSGTPRT |
| 1088 | P LCDR3 | X₁QWX₂X₃X₄PRT |
| | | X₁ = Q OR N |
| | | X₂ = D, E, H, N, Q, OR S |
| | | X₃ = A, OR G |
| | | X₄ = D, F, K, N, R, S, OR T |
| 1089 | P LCDR3 | X₁QX₂X₃X₄X₅PRX₆ |
| | | X₁ = H, N, Q, or S |
| | | X₂ = F, H, I, P, R, S, W, |
| | | or Y |
| | | X₃ = D, E, H, N, Q, S, or V |
| | | X₄ = A, D, G, Q, or S |
| | | X₅ = D, F, H, K, L, M, N, Q, |
| | | R, S, or T |
| | | X₆ = T or S |
| 1090 | M1 LCDR3 | QQYYSTPFT |

| | | |
|---|---|---|
| 1091 | M2 LCDR3 | MQATQFPLT |
| 1092 | M3 LCDR3 | QQRSNWPWT |
| 1093 | M4 LCDR3 | QQFNSYPLT |
| 1094 | M5 LCDR3 | HQWNNYGT |
| 1095 | M6 LCDR3 | FQGSHVPLT |
| 1096 | M7 LCDR3 | QQGQSYPYT |
| 1097 | M8 LCDR3 | QSXDGTLSAL; X = Y, W, F |
| 1098 | M9 LCDR3 | QQSYSTPRT |
| 1099 | M10 LCDR3 | QQYGSSPT |
| 1100 | M11 LCDR3 | QQYHRSWT |
| 1101 | M12 LCDR3 | FQGSHVPLT |

TABLE 16
Heavy Chain Variable Region (VH) Amino Acid Sequences

| SEQ ID | | |
|---|---|---|
| NO | Description | Sequence |
| 101 | 217 VH, 158 VH | QVQLVQSGAEVKKPGASVKVSCKASGFDIQDTYMH |
| | | WVKQRPGQGLEWMGRIDPASGHTKYDPKFQVRVTI |
| | | TRDTSTSTVYMELSSLRSEDTAVYYCARSGGLPDV |
| | | WGQGTTVTVSS |
| 102 | 220 VH, 160 VH | QVQLVQSGAEVKKPGASVKVSCKASGFDIQDTYMH |
| | | WVKQRPGQGLEWMGRIDPASGHTKYDPKFQVRVT |
| | | MTRDTSTSTAYLELSSLRSEDTAVYYCARSGGLPDV |
| | | WGQGTTVTVSS |
| 103 | 223 VH, 200 VH, | EVQLVQSGAEVKKPGASVKVSCKASGFDIQDTYMH |
| | 194 VL, 206 VH | WVRQRPGQGLEWIGRIDPASGHTKYDPKFQVRATIT |
| | | TDTSTSTAYLELSSLRSEDTAVYYCARSGGLPDVWG |
| | | QGTTVTVSS |
| 104 | 219 VH, 157 VH | QVQLVQSGAEVKKPGASVKVSCKASGFDIQDTYMH |
| | | WVKQRPGQGLEWMGRIDPASGHTKYDPKFQVRVTI |
| | | TRDTSTSTVYLELSSLRSEDTAVYYCARSGGLPDVW |
| | | GQGTTVTVSS |
| 105 | 221 VH, 125 VH | QVQLVQSGAEVKKPGASVKVSCKASGFDIQDTYMH |
| | | WVKQRPGQGLEWMGRIDPASGHTKYDPKFQVRATI |
| | | TRDTSTSTAYLELSSLRSEDTAVYYCARSGGLPDVW |
| | | GQGTTVTVSS |
| 106 | 213 VH, 162 VH | QVQLVQSGAEVKKPGASVKVSCKASGFDIQDTYMH |
| | | WVRQRPGQGLEWMGRIDPASGHTKYDPKFQVRVTI |
| | | TTDTSTSTVYMELSSLRSEDTAVYYCARSGGLPDV |
| | | WGQGTTVTVSS |
| 107 | 212 VH, 100 VH, | QVQLVQSGAEVKKPGASVKVSCKASGFDIQDTYMH |
| | 181 VH, 34 VH, 79 | WVRQRPGQGLEWIGRIDPASGHTKYDPKFQVRATIT |
| | VH | TDTSTSTAYLELSSLRSEDTAVYYCARSGGLPDVWG |
| | | QGTTVTVSS |
| 108 | 107 VH, 211 VH, | QVQLVQSGAEVKKPGASVKVSCKASGFDIQDTYMH |
| | 15 VH, 30 VH, 29 | WVKQRPGQGLEWIGRIDPASGHTKYDPKFQVRATIT |
| | VH, 48 VH, 49 VH, | TDTSTSTAYLELSSLRSEDTAVYYCARSGGLPDVWG |
| | 50 VH, 51 VH, 52 | QGTTVTVSS |
| | VH, 56 VH | |
| 109 | 205 VH, 199 VH, | EVQLVQSGAEVKKPGASVKVSCKASGFDIQDTYMH |
| | 55 VH, 193 VH | WVKQRPGQGLEWIGRIDPASGHTKYDPKFQVRATIT |
| | | TDTSTSTAYLELSSLRSEDTAVYYCARSGGLPDVWG |
| | | QGTTVTVSS |
| 110 | 129 VH, 139 VH, | QVQLVQSGAEVKKPGASVKVSCKASGFDIQDTYMH |
| | 140 VH, 215 VH | WVRQRPGQGLEWMGRIDPASGHTKYDPKFQVRVTI |
| | | TTDTSTSTAYLELSSLRSEDTAVYYCARSGGLPDVW |
| | | GQGTTVTVSS |
| 111 | 214 VH, 128 VH, | QVQLVQSGAEVKKPGASVKVSCKASGFDIQDTYMH |
| | 141 VH, 142 VH, | WVRQRPGQGLEWMGRIDPASGHTKYDPKFQVRVTI |
| | 144 VH | TRDTSTSTAYLELSSLRSEDTAVYYCARSGGLPDVW |
| | | GQGTTVTVSS |
| 112 | 216 VH, 123 VH | QVQLVQSGAEVKKPGASVKVSCKASGFDIQDTYMH |
| | | WVRQRPGQGLEWIGRIDPASGHTKYDPKFQVRVTIT |
| | | RDTSTSTAYLELSSLRSEDTAVYYCARSGGLPDVWG |
| | | QGTTVTVSS |
| 113 | 122 VH | QVQLVQSGAEVKKPGASVKVSCKASGFDIQDTYMH |
| | | WVRQRPGQGLEWIGRIDPASGHTKYDPKFQVRATIT |
| | | RDTSTSTAYLELSSLRSEDTAVYYCARSGGLPDVWG |
| | | QGTTVTVSS |
| 114 | 222 VH, 126 VH | QVQLVQSGAEVKKPGASVKVSCKASGFDIQDTYMH |
| | | WVKQRPGQGLEWMGRIDPASGHTKYDPKFQVRVTI |
| | | TRDTSTSTAYLELSSLRSEDTAVYYCARSGGLPDVW |
| | | GQGTTVTVSS |
| 115 | 188 VH, 41 VH, | QVQLVQSGAEVKKPGASVKVSCKASGFDIQDTYMH |
| | 102 VH | WVKQRPGQGLEWIGRIDPASGHTKYDPKFQVRVTIT |
| | | TDTSTSTAYLELSSLRSEDTAVYYCARSGGLPDVWG |
| | | QGTTVTVSS |
| 116 | 203 VH, 197 VH, | EVQLVQSGAEVKKPGASVKVSCKASGFDIQDTYMH |
| | 209 VH, 224 VH | WVKQAPGQGLEWMGRIEPASGHIKYDPKFQGRVT |
| | | MTRDTSTSTVYMELSSLRSEDTAVYYCARSGGLPD |
| | | WWGQGTTVTVSS |
| 117 | 147 VH, 112 VH, | QVQLVQSGAEVKKPGASVKVSCKASGFDIQDTYMH |
| | 59 VH | WVKQRPGQGLEWMGRIEPASGHIKYDPKFQGRVT |
| | | MTRDTSTSTVYMELSSLRSEDTAVYYCARSGGLPD |
| | | WWGQGTTVTVSS |
| 118 | 127 VH | QVQLVQSGAEVKKPGASVKVSCKASGFDIQDTYMH |
| | | WVKQRPGQGLEWMGRIDPASGHTKYDPKFQVRVTI |
| | | TTDTSTSTAYLELSSLRSEDTAVYYCARSGGLPDVW |
| | | GQGTTVTVSS |
| 119 | 159 VH, 218 VH | QVQLVQSGAEVKKPGASVKVSCKASGFDIQDTYMH |
| | | WVKQRPGQGLEWMGRIDPASGHTKYDPKFQVRVTI |
| | | TRDTSTSTAYMELSSLRSEDTAVYYCARSGGLPDV |
| | | WGQGTTVTVSS |
| 120 | 103 VH, 45 VH, | QVQLVQSGAEVKKPGASVKVSCKASGFDIQDTYMH |
| | 167 VH, 187 VH | WVKQRPGQGLEWIGRIDPASGHTKYDPKFQVRVTIT |
| | | RDTSTSTAYLELSSLRSEDTAVYYCARSGGLPDVWG |
| | | QGTTVTVSS |
| 121 | 64 VH, 148 VH, 97 | QVQLVQSGAEVKKPGASVKVSCKASGFDIQDTYMH |
| | VH, 114 VH, 130 | WVRQAPGQGLEWMGRIEPASGHIKYDPKFQVRVT |
| | VH, 133 VH, 137 | MTRDTSTSTVYMELSSLRSEDTAVYYCARSGGLPD |
| | VH, 155 VH | WWGQGTTVTVSS |
| 122 | 67 VH, 138 VH, | QVQLVQSGAEVKKPGASVKVSCKASGFDIQDTYMH |
| | 115 VH, 149 VH, | WVRQAPGQGLEWMGRIEPASGHIKYDPKFQVRAT |
| | 134 VH, 98 VH, | MTRDTSTSTVYMELSSLRSEDTAVYYCARSGGLPD |
| | 156 VH | WWGQGTTVTVSS |
| 123 | 68 VH, 99 VH, 116 | QVQLVQSGAEVKKPGASVKVSCKASGFDIQDTYMH |
| | VH | WVRQAPGQGLEWMGRIEPASGHIKYDPKFQVRVTI |
| | | TRDTSTSTVYMELSSLRSEDTAVYYCARSGGLPDW |
| | | WGQGTTVTVSS |
| 124 | 94 VH, 113 VH, | QVQLVQSGAEVKKPGASVKVSCKASGFDIQDTYMH |
| | 151 VH, 78 VH | WVRQAPGQGLEWMGRIEPASGHIKYDPKFQVRATI |
| | | TRDTSTSTVYMELSSLRSEDTAVYYCARSGGLPDW |
| | | WGQGTTVTVSS |
| 125 | 110 VH, 58 VH, | QVQLVQSGAEVKKPGASVKVSCKASGFDIQDTYMH |
| | 136 VH, 146 VH, | WVKQAPGQGLEWMGRIEPASGHIKYDPKFQGRVT |
| | 154 VH | MTRDTSTSTVYMELSSLRSEDTAVYYCARSGGLPD |
| | | WWGQGTTVTVSS |
| 126 | 169 VH, 175 VH | QVQLVQSGAEVKKPGASVKVSCKASGFDIQDTYMH |
| | | WVKQAPGQGLEWMGRIDPASGHIKYDPKFQGRVT |
| | | MTRDTSTSTVYMELSSLRSEDTAVYYCARSGGLPD |
| | | WWGQGTTVTVSS |
| 127 | 173 VH, 179 VH | QVQLVQSGAEVKKPGASVKVSCKASGFDIQDTYMH |
| | | WVKQAPGQGLEWMGRIEPASGHIKYDPKFQGRAT |
| | | MTRDTSTSTVYMELSSLRSEDTAVYYCARSGGLPD |
| | | WWGQGTTVTVSS |
| 128 | 96 VH, 132 VH, 65 | QVQLVQSGAEVKKPGASVKVSCKASGFDIQDTYMH |
| | VH, 150 VH | WVRQAPGQGLEWMGRIEPASGHIKYDPKFQGRAT |
| | | MTRDTSTSTVYMELSSLRSEDTAVYYCARSGGLPD |
| | | WWGQGTTVTVSS |
| 129 | 196 VH, 202 VH, | EVQLVQSGAEVKKPGASVKVSCKASGFDIQDTYMH |
| | 208 VH | WVRQAPGQGLEWMGRIEPASGHIKYDPKFQGRAT |
| | | MTRDTSTSTVYMELSSLRSEDTAVYYCARSGGLPD |
| | | WWGQGTTVTVSS |
| 130 | 172 VH, 178 VH | QVQLVQSGAEVKKPGASVKVSCKASGFDIQDTYMH |
| | | WVRQAPGQGLEWMGRIDPASGHIKYDPKFQGRAT |
| | | MTRDTSTSTVYMELSSLRSEDTAVYYCARSGGLPD |
| | | WWGQGTTVTVSS |
| 131 | 75 VH, 72 VH, 95 | QVQLVQSGAEVKKPGASVKVSCKASGFDIQDTYMH |
| | VH, 152 VH | WVRQAPGQGLEWMGRIEPASGHIKYDPKFQGRATI |
| | | TTDTSTSTVYMELSSLRSEDTAVYYCARSGGLPDW |
| | | WGQGTTVTVSS |
| 132 | 174 VH, 180 VH | QVQLVQSGAEVKKPGASVKVSCKASGFDIQDTYMH |
| | | WVRQAPGQGLEWMGRIEPASGHIKYDPKFQGRAT |
| | | MTRDTSTSTAYMELSSLRSEDTAVYYCARSGGLPD |
| | | WWGQGTTVTVSS |
| 133 | 109 VH, 91 VH, | QVQLVQSGAEVKKPGASVKVSCKASGFDIQDTYMH |
| | 135 VH, 145 VH, | WVRQAPGQGLEWMGRIEPASGHIKYDPKFQGRVT |
| | 153 VH | MTRDTSTSTAYMELSSLRSEDTAVYYCARSGGLPD |
| | | WWGQGTTVTVSS |
| 134 | 198 VH, 204 VH, | EVQLVQSGAEVKKPGASVKVSCKASGFDIQDTYMH |
| | 210 VH | WVRQAPGQGLEWMGRIEPASGHIKYDPKFQGRVT |
| | | MTRDTSTSTAYMELSSLRSEDTAVYYCARSGGLPD |
| | | WWGQGTTVTVSS |
| 135 | 170 VH, 176 VH | QVQLVQSGAEVKKPGASVKVSCKASGFDIQDTYMH |
| | | WVRQAPGQGLEWMGRIDPASGHIKYDPKFQGRVT |
| | | MTRDTSTSTAYMELSSLRSEDTAVYYCARSGGLPD |
| | | WWGQGTTVTVSS |
| 136 | 31 VH, 85 VH, 86 | QVQLVQSGAEVKKPGASVKVSCKASGFDIQDTYMH |
| | VH, 87 VH, 88 VH, | WVRQAPGQGLEWMGRIEPASGHIKYDPKFQGRVT |
| | 89 VH, 90 VH, 143 | MTRDTSTSTVYMELSSLRSEDTAVYYCARSGGLPD |
| | VH, clone 34 VH | WWGQGTTVTVSS |
| 137 | 32 VH, 33 VH | DVQLVQSGAEVKKPGASVKVSCKASGFDIQDTYMH |
| | | WVRQAPGQGLEWMGRIEPASGHIKYDPKFQGRVT |
| | | MTRDTSTSTVYMELSSLRSEDTAVYYCARSGGLPD |
| | | WWGQGTTVTVSS |
| 138 | 35 VH, 182 VH | QVQLVQSGAEVKKPGASVKVSCKASGFDIQDTYMH |
| | | WVKQAPGQGLEWIGRIDPASGHTKYDPKFQVRATI |
| | | TTDTSTSTAYLELSSLRSEDTAVYYCARSGGLPDVW |
| | | GQGTTVTVSS |
| 139 | 36 VH, 81 VH, 104 | QVQLVQSGAEVKKPGASVKVSCKASGFDIQDTYMH |
| | VH, 165 VH, | WVRQAPGQGLEWIGRIDPASGHTKYDPKFQVRATIT |
| | | TDTSTSTAYLELSSLRSEDTAVYYCARSGGLPDVWG |
| | | QGTTVTVSS |
| 140 | 37 VH, 82 VH, 101 | QVQLVQSGAEVKKPGASVKVSCKASGFDIQDTYMH |
| | VH, 183 VH | WVKQRPGQGLEWMGRIDPASGHTKYDPKFQVRATI |
| | | TTDTSTSTAYLELSSLRSEDTAVYYCARSGGLPDVW |
| | | GQGTTVTVSS |
| 141 | 38 VH, 190 VH | QVQLVQSGAEVKKPGASVKVSCKASGFDIQDTYMH |
| | | WVKQRPGQGLEWIGRIDPASGHTKYDPKFQVRATIT |
| | | TDTSTSTVYLELSSLRSEDTAVYYCARSGGLPDVWG |
| | | QGTTVTVSS |
| 142 | 39 VH, 191 VH | QVQLVQSGAEVKKPGASVKVSCKASGFDIQDTYMH |
| | | WVKQRPGQGLEWIGRIDPASGHTKYDPKFQVRATIT |
| | | TDTSTSTAYMELSSLRSEDTAVYYCARSGGLPDVW |
| | | GQGTTVTVSS |
| 143 | 40 VH, 105 VH, | QVQLVQSGAEVKKPGASVKVSCKASGFDIQDTYMH |
| | 192 VH | WVKQRPGQGLEWIGRIDPASGHTKYDPKFQVRATIT |
| | | TDTSTSTVYMELSSLRSEDTAVYYCARSGGLPDVW |
| | | GQGTTVTVSS |
| 144 | 42 VH, 83 VH, 186 | QVQLVQSGAEVKKPGASVKVSCKASGFDIQDTYMH |
| | VH | WVKQRPGQGLEWIGRIDPASGHTKYDPKFQVRATM |
| | | TTDTSTSTAYLELSSLRSEDTAVYYCARSGGLPDVW |
| | | GQGTTVTVSS |
| 145 | 43 VH, 184 VH | QVQLVQSGAEVKKPGASVKVSCKASGFDIQDTYMH |
| | | WVKQRPGQGLEWIGRIDPASGHTKYDPKFQVRATIT |
| | | RDTSTSTAYLELSSLRSEDTAVYYCARSGGLPDVWG |
| | | QGTTVTVSS |
| 146 | 44 VH, 53 VH, 166 | QVQLVQSGAEVKKPGASVKVSCKASGFDIQDTYMH |
| | VH, 189 VH | WVKQRPGQGLEWIGRIDPASGHTKYDPKFQVRVTM |
| | | TTDTSTSTAYLELSSLRSEDTAVYYCARSGGLPDVW |
| | | GQGTTVTVSS |
| 147 | 46 VH, 168 VH, | QVQLVQSGAEVKKPGASVKVSCKASGFDIQDTYMH |
| | 185 VH | WVKQRPGQGLEWIGRIDPASGHTKYDPKFQVRATM |
| | | TRDTSTSTAYLELSSLRSEDTAVYYCARSGGLPDVW |
| | | GQGTTVTVSS |
| 148 | 47 VH | QVQLVQSGAEVKKPGASVKVSCKASGFDIQDTYMH |
| | | WVKQRPGQGLEWIGRIDPASGHTKYDPKFQVRVTM |
| | | TRDTSTSTAYLELSSLRSEDTAVYYCARSGGLPDVW |
| | | GQGTTVTVSS |
| 149 | 54 VH | DVQLVQSGAEVKKPGASVKVSCKASGFDIQDTYMH |
| | | WVKQRPGQGLEWIGRIDPASGHTKYDPKFQVRATIT |
| | | TDTSTSTAYLELSSLRSEDTAVYYCARSGGLPDVWG |
| | | QGTTVTVSS |
| 150 | 57 VH, 111 VH | QVQLVQSGAEVKKPGASVKVSCKASGFDIQDTYMH |
| | | WVRQRPGQGLEWMGRIEPASGHIKYDPKFQGRVT |
| | | MTRDTSTSTVYMELSSLRSEDTAVYYCARSGGLPD |
| | | WWGQGTTVTVSS |
| 151 | 60 VH, 117 VH | QVQLVQSGAEVKKPGASVKVSCKASGFDIQDTYMH |
| | | WVRQAPGQGLEWMGRIDPASGHIKYDPKFQGRVT |
| | | MTRDTSTSTVYMELSSLRSEDTAVYYCARSGGLPD |
| | | WWGQGTTVTVSS |
| 152 | 61 VH | QVQLVQSGAEVKKPGASVKVSCKASGFDIQDTYMH |
| | | WVRQAPGQGLEWIGRIEPASGHIKYDPKFQGRVTM |
| | | TRDTSTSTVYMELSSLRSEDTAVYYCARSGGLPDW |
| | | WGQGTTVTVSS |
| 153 | 62 VH, 118 VH | QVQLVQSGAEVKKPGASVKVSCKASGFDIQDTYMH |
| | | WVRQAPGQGLEWIGRIDPASGHIKYDPKFQGRVTM |
| | | TRDTSTSTVYMELSSLRSEDTAVYYCARSGGLPDW |
| | | WGQGTTVTVSS |
| 154 | 63 VH, 120 VH | QVQLVQSGAEVKKPGASVKVSCKASGFDIQDTYMH |
| | | WVRQAPGQGLEWMGRIEPASGHVKYDPKFQGRVT |
| | | MTRDTSTSTVYMELSSLRSEDTAVYYCARSGGLPD |
| | | WWGQGTTVTVSS |
| 155 | 66 VH | QVQLVQSGAEVKKPGASVKVSCKASGFDIQDTYMH |
| | | WVRQAPGQGLEWMGRIEPASGHIKYDPKFQGRVTI |
| | | TRDTSTSTVYMELSSLRSEDTAVYYCARSGGLPDW |
| | | WGQGTTVTVSS |
| 156 | 69 VH, 108 VH | EVQLVQSGAEVKKPGASVKVSCKASGFDIQDTYMH |
| | | WVRQAPGQGLEWMGRIEPASGHIKYDPKFQGRVT |
| | | MTRDTSTSTVYMELSSLRSEDTAVYYCARSGGLPD |
| | | WWGQGTTVTVSS |
| 157 | 70 VH, 73 VH | QVQLVQSGAEVKKPGASVKVSCKASGFDIQDTYMH |
| | | WVRQAPGQGLEWMGRIEPASGHIKYDPKFQGRVT |
| | | MTTDTSTSTVYMELSSLRSEDTAVYYCARSGGLPD |
| | | WWGQGTTVTVSS |
| 158 | 71 VH, 74 VH | QVQLVQSGAEVKKPGASVKVSCKASGFDIQDTYMH |
| | | WVRQAPGQGLEWMGRIEPASGHIKYDPKFQGRVTI |
| | | TTDTSTSTVYMELSSLRSEDTAVYYCARSGGLPDW |
| | | WGQGTTVTVSS |
| 159 | 76 VH, 119 VH | QVQLVQSGAEVKKPGASVKVSCKASGFDIQDTYMH |
| | | WVRQAPGQGLEWMGRIEPASGHTKYDPKFQGRVT |
| | | MTRDTSTSTVYMELSSLRSEDTAVYYCARSGGLPD |
| | | WWGQGTTVTVSS |
| 160 | 77 VH | QVQLVQSGAEVKKPGASVKVSCKASGFDIQDTYMH |
| | | WVRQAPGQGLEWMGRIEPASGHIKYDPKFQGRATI |
| | | TRDTSTSTVYMELSSLRSEDTAVYYCARSGGLPDW |
| | | WGQGTTVTVSS |
| 161 | 92 VH | QVQLVQSGAEVKKPGASVKVSCKASGFDIQDTYMH |
| | | WVRQAPGQGLEWMGRIEPASGHIKYDPKFQGRVT |
| | | MTRDTSTSTVYLELSSLRSEDTAVYYCARSGGLPD |
| | | WWGQGTTVTVSS |
| 162 | 93 VH | QVQLVQSGAEVKKPGASVKVSCKASGFDIQDTYMH |
| | | WVRQAPGQGLEWMGRIEPASGHIKYDPKFQGRVT |
| | | MTRDTSTSTAYLELSSLRSEDTAVYYCARSGGLPD |
| | | WWGQGTTVTVSS |
| 163 | 121 VH | QVQLVQSGAEVKKPGASVKVSCKASGFDIQDTYMH |
| | | WVRQRPGQGLEWMGRIDPASGHTKYDPKFQVRATI |
| | | TTDTSTSTAYLELSSLRSEDTAVYYCARSGGLPDVW |
| | | GQGTTVTVSS |
| 164 | 124 VH | QVQLVQSGAEVKKPGASVKVSCKASGFDIQDTYMH |
| | | WVRQRPGQGLEWIGRIDPASGHTKYDPKFQVRVTIT |
| | | TDTSTSTAYLELSSLRSEDTAVYYCARSGGLPDVWG |
| | | QGTTVTVSS |
| 165 | 161 VH | QVQLVQSGAEVKKPGASVKVSCKASGFDIQDTYMH |
| | | WVRQRPGQGLEWMGRIDPASGHTKYDPKFQVRVTI |
| | | TTDTSTSTVYLELSSLRSEDTAVYYCARSGGLPDVW |
| | | GQGTTVTVSS |
| 166 | 163 VH | QVQLVQSGAEVKKPGASVKVSCKASGFDIQDTYMH |
| | | WVRQRPGQGLEWMGRIDPASGHTKYDPKFQVRVTI |
| | | TTDTSTSTAYMELSSLRSEDTAVYYCARSGGLPDV |
| | | WGQGTTVTVSS |
| 167 | 164 VH | QVQLVQSGAEVKKPGASVKVSCKASGFDIQDTYMH |
| | | WVRQRPGQGLEWMGRIDPASGHTKYDPKFQVRVT |
| | | MTTDTSTSTAYLELSSLRSEDTAVYYCARSGGLPDV |
| | | WGQGTTVTVSS |
| 168 | 171 VH, 177 VH | QVQLVQSGAEVKKPGASVKVSCKASGFDIQDTYMH |
| | | WVRQAPGQGLEWMGRIDPASGHIKYDPKFQGRATI |
| | | TTDTSTSTVYMELSSLRSEDTAVYYCARSGGLPDW |
| | | WGQGTTVTVSS |
| 169 | 195 VH, 201 VH, | EVQLVQSGAEVKKPGASVKVSCKASGFDIQDTYMH |
| | 207 VH | WVRQAPGQGLEWMGRIEPASGHIKYDPKFQGRATI |
| | | TTDTSTSTVYMELSSLRSEDTAVYYCARSGGLPDW |
| | | WGQGTTVTVSS |
| 1200 | 5C3D11 VH | EVQLQQSGAELVKPGASVKLSCTASGFDIQDTYMH |
| | | WVKQRPEQGLEWIGRIDPASGHTKYDPKFQVKATIT |
| | | TDTSSNTAYLQLSSLTSEDTAVYYCSRSGGLPDVWG |
| | | AGTTVTVSS |
| 1201 | 9E12ES VH | QVHLQQSGPELVKPGASVKLSCKASGYTFTKYDIN |
| | | WVRQRPEQGLEWIGWIFPGDGRTDYNEKFKGKATL |
| | | TTDKSSSTAYMEVSRLTSEDSAVYFCARYGPAMDY |
| | | WGQGTSVTVAS |
| 1202 | AS12824 VH | QVQLVQSGAEVKKPGASVKVSCKASGFDICDTYMH |
| | | WVKQRPGQGLEWIGRIDPASGHTKYDPKFQVRATIT |
| | | TDTSTSTAYLELSSLRSEDTAVYYCARSGGLPDVWG |
| | | QGTTVTVSS |
| 1203 | AS12823 VH | QVQLVQSGAEVKKPGASVKLSCKASGFDICDTYMH |
| | | WVRQRPGQGLEWIGRIDPASGHTKYDPKFQVRATM |
| | | TTDTSTSTVYLELSSLRSEDTAVYYCARSGGLPDVW |
| | | GQGTTVTVSS |
| 1204 | AS12819 VH | QVQLVQSGAEVVKPGASVKLSCKASGFDICDTYMH |
| | | WVRQRPGQGLEWMGRIDPASGHTKYDPKFQVRVT |
| | | MTTDTSTSTVYLELSSLRSEDTAVYYCARSGGLPDV |
| | | WGQGTTVTVSS |
| 1205 | AS12816 VH | QVQLVQSGAEVKKPGASVKVSCKASGFDICDTYMH |
| | | WVKQRPGQGLEWIGRIDPASGHTKYDPKFQVRATIT |
| | | RDTSTSTAYLELSSLRSEDTAVYYCSRSGGLPDVWG |
| | | QGTTVTVSS |
| 1206 | AS12825 VH | QVQLVQSGAEVKKPGASVKVSCKASGFDICDTYMH |
| | | WVKQAPGQGLEWMGRIDPASGHTKYDPKFQVRAT |
| | | MTTDTSTSTAYLELSSLRSEDTAVYYCSRSGGLPDV |
| | | WGQGTTVTVSS |
| 1207 | 12835 VH | QVQLVQSGAEVKKPGASVKLSCKASGFDIQDTYMH |
| | | WVRQAPGQGLEWMGRIDPASGHTKYDPKFQVRVT |
| | | MTTDTSTSTVYMELSSLRSEDTAVYYCSRSGGLPDV |
| | | WGQGTTVTVSS |
| 1208 | 18-7 VH | QVQLVQSGAEVKKPGASVKLSCKASGFDIQDTYMH |
| | | WVRQAPGQGLEWMGRIDPASGHTKYDPKFQVRVT |
| | | MTRDTSTSTVYMELSSLRSEDTAVYYCSRSGGLPDV |
| | | WGQGTTVTVSS |
| 1209 | 21-3 VH, L8 VH | QVQLVQSGAEVKKPGASVKVSCKASGFDIQDTYMH |
| | | WVRQAPGQGLEWMGRIDPASGHTKYDPKFQVRVT |
| | | MTRDTSTSTVYMELSSLRSEDTAVYYCARSGGLPD |
| | | VWGQGTTVTVSS |
| 1210 | 21-3 V102K VH | QVQLVQSGAEVKKPGASVKVSCKASGFDIQDTYMH |
| | | WVRQAPGQGLEWMGRIDPASGHTKYDPKFQVRVT |
| | | MTRDTSTSTVYMELSSLRSEDTAVYYCARSGGLPD |
| | | KWGQGTTVTVSS |
| 1211 | 21-3 V102M VH | QVQLVQSGAEVKKPGASVKVSCKASGFDIQDTYMH |
| | | WVRQAPGQGLEWMGRIDPASGHTKYDPKFQVRVT |
| | | MTRDTSTSTVYMELSSLRSEDTAVYYCARSGGLPD |
| | | MWGQGTTVTVSS |
| 1212 | 21-3 V102Q VH | QVQLVQSGAEVKKPGASVKVSCKASGFDIQDTYMH |
| | | WVRQAPGQGLEWMGRIDPASGHTKYDPKFQVRVT |
| | | MTRDTSTSTVYMELSSLRSEDTAVYYCARSGGLPD |
| | | QWGQGTTVTVSS |
| 1213 | 21-3 V102W VH | QVQLVQSGAEVKKPGASVKVSCKASGFDIQDTYMH |
| | | WVRQAPGQGLEWMGRIDPASGHTKYDPKFQVRVT |
| | | MTRDTSTSTVYMELSSLRSEDTAVYYCARSGGLPD |
| | | WWGQGTTVTVSS |
| 1214 | 21-3 CDRv VH | QVQLVQSGAEVKKPGASVKVSCKASGFX₁X₂X₃DT |
| | | X₄X₅HWVRQAPGQGLEWMGRIDPASGHTKYDPKFQV |
| | | RVTMTRDTSTSTVYMELSSLRSEDTAVYYCARSGG |
| | | X₆PDX₇WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 1215 | 21-3 CDRv VH | QVQLVQSGAEVKKPGASVKVSCKASGFX₁X₂X₃DT |
| | | X₄X₅HWVRQAPGQGLEWMGRIDPASGHTKYDPKFQV |
| | | RVTMTRDTSTSTVYMELSSLRSEDTAVYYCSRSGG |
| | | X₆PDX₇WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 1216 | Clone 2 VH, clone | QVQLVQSGAEVKKPGASVKVSCKASGFDIQDTYMH |
| | 52 VH | WVRQAPGQGLEWMGRIEPASGHIKYSPKFQGRVTM |
| | | TRDTSTSTVYMELSSLRSEDTAVYYCARSGGLPDW |
| | | WGQGTTVTVSS |
| 1217 | Clone 46 VH | QVQLVQSGAEVKKPGASVKVSCKASGFDIQDTYMH |
| | | WVRQAPGQGLEWMGRIEPASGHVKYSPKFQVRVT |
| | | MTRDTSTSTVYMELSSLRSEDTAVYYCARSGGLPD |
| | | WWGQGTTVTVSS |
| 1218 | Clone 47 VH | QVQLVQSGAEVKKPGASVKVSCKASGFDIQDTYMH |
| | | WVRQAPGQGLEWMGRIEPASGHVKYDPKFQTRVT |
| | | MTRDTSTSTVYMELSSLRSEDTAVYYCARSGGLPD |
| | | WWGQGTTVTVSS |
| 1219 | Clone 14 VH | QVQLVQSGAEVKKPGASVKVSCKASGFDIQDTYMH |
| | | WVRQAPGQGLEWMGRIDPASGHIKYDPKFQKRVTM |
| | | TRDTSTSTVYMELSSLRSEDTAVYYCARSGGLPDM |
| | | WGQGTTVTVSS |
| 1220 | Clone 16L VH | QVQLVQSGAEVKKPGASVKVSCKASGFDIQDTYMH |
| | | WVRQAPGQGLEWMGRIDPASGHvKIDPKFQVRVTM |
| | | TRDTSTSTVYMELSSLRSEDTAVYYCARSGGLPDM |
| | | WGQGTTVTVSS |
| 1221 | Clone 17L VH | QVQLVQSGAEVKKPGASVKVSCKASGFDIQDTYMH |
| | | WVRQAPGQGLEWMGRIDPASGHLKYDPKFQVRVT |
| | | MTRDTSTSTVYMELSSLRSEDTAVYYCARSGGLPD |
| | | MWGQGTTVTVSS |
| 1222 | Clone 17L-1 VH | QVQLVQSGAEVKKPGASVKVSCKASGFDIQDTYMH |
| | | WVRQAPGQGLEWMGRIDPASGHLKYDPKFQRRVT |
| | | MTRDTSTSTVYMELSSLRSEDTAVYYCARSGGLPD |
| | | MWGQGTTVTVSS |
| 1223 | Clone 23 VH, clone | QVQLVQSGAEVKKPGASVKVSCKASGFDIQDTYMH |
| | A1 VH | WVRQAPGQGLEWMGRIDPASGHLKYDPKFQNRVT |
| | | MTRDTSTSTVYMELSSLRSEDTAVYYCARSGGLPD |
| | | KWGQGTTVTVSS |
| 1224 | Clone 53 VH, clone | QVQLVQSGAEVKKPGASVKVSCKASGFDIQDTYMH |
| | E1 VH | WVRQAPGQGLEWMGRIEPASGHLKYDPKFQERVT |
| | | MTRDTSTSTVYMELSSLRSEDTAVYYCARSGGLPD |
| | | KWGQGTTVTVSS |
| 1225 | Clone 3-17L V-A | QVQLVQSGAEVKKPGASVKVSCKASGFDIQDTYMH |
| | VH | WVRQAPGQGLEWMGRIDPASGHLKYDPKFQGRVTI |
| | | TRDTSASTAYMELSSLRSEDTAVYYCARSGGLPDM |
| | | WGQGTTVTVSS |
| 1226 | Clone 3-17L VH | QVQLVQSGAEVKKPGASVKVSCKASGFDIQDTYMH |
| | | WVRQAPGQGLEWMGRIDPASGHLKYDPKFQGRVTI |
| | | TRDTSASTVYMELSSLRSEDTAVYYCARSGGLPDM |
| | | WGQGTTVTVSS |
| 1227 | Clone L8mod VH | QVQLVQSGAEVKKPGASVKVSCKASGFDIQDTYMH |
| | | WVRQAPGQGLEWMGRIDPASGHTKYDPKFQGRATI |
| | | TTDTSASTAYLQLSSLRSEDTAVYYCARSGGLPDV |
| | | WGQGTTVTVSS |
| 1228 | Clone X-V VH | QVQLVQSGAEVKKPGASVKVSCKASGFDIQDTYMH |
| | | WVRQAPGQGLEWMGRIDPASGHTKYDPKFQVRATI |
| | | TTDTSASTAYLQLSSLRSEDTAVYYCARSGGLPDFW |
| | | GQGTTVTVSS |
| 1229 | Clone X VH, clone | QVQLVQSGAEVKKPGASVKVSCKASGFDIQDTYMH |
| | XL3-6 VH, clone | WVRQAPGQGLEWMGRIDPASGHTKYDPKFQGRATI |
| | XL3-10 VH, clone | TTDTSASTAYLQLSSLRSEDTAVYYCARSGGLPDFW |
| | XL3-15 VH, clone | GQGTTVTVSS |
| | L3-13 VH | |
| 1230 | Clone H3-1 VH | QVQLVQSGAEVKKPGASVKVSCKASGFDIQDTYMH |
| | | WVRQAPGQGLEWMGRIDPASGHTKYDPKFQGRATI |
| | | TTDTSASTAYLQLSSLRSEDTAVYYCARSGGLPDLW |
| | | GQGTTVTVSS |
| 1231 | Clone H2-2 VH | QVQLVQSGAEVKKPGASVKVSCKASGFDIQDTYMH |
| | | WVRQAPGQGLEWMGRIDPASGHSKYDPKFQVRATI |
| | | TTDTSASTAYLQLSSLRSEDTAVYYCARSGGLPDFW |
| | | GQGTTVTVSS |
| 1232 | Clone H2-S VH | QVQLVQSGAEVKKPGASVKVSCKASGFDIQDTYMH |
| | | WVRQAPGQGLEWMGRIDPASGHYKYDPKFQVRAT |
| | | ITTDTSASTAYLQLSSLRSEDTAVYYCARSGGLPDF |
| | | WGQGTTVTVSS |
| 1233 | M1 VH | EVQLVESGGGLVQPGGSLRLSCAVSGFTFSSYWMS |
| | | WVRQAPGKGLEWVANIKEDGSEKNYVDSVKGRFT |
| | | LSSDNAKNSLYLQMNSLRAEDTAVYYCAREDYDSY |
| | | YKYGMDVWGQGTAVIVSS |
| 1234 | M2 VH | QVQLQQWGAGLLKPSETLSLTCAVYGGSFTGFYWS |
| | | WIRQPPGKGLEWIGEINHRGNTNYNPSLKSRVTMSV |
| | | DTSKNQFSLNMISVTAADTAMYFCASPFYDFWSGS |
| | | DYWGQGTLVTVSS |
| 1235 | M3 VH | QVQLVQSGAEVKKPGASVKVSCKASGYDFTYYGIS |
| | | WVRQAPGQGLEWMGWISTYNGNTHYARMLQGRV |
| | | TMTTDTSTRTAYMELRSLRSDDTAVYYCARENYYG |
| | | SGAYRGGMDVWGQGTTVTVSS |
| 1236 | M3 VH + constant | QVQLVQSGAEVKKPGASVKVSCKASGYDFTYYGIS |
| | | WVRQAPGQGLEWMGWISTYNGNTHYARMLQGRV |
| | | TMTTDTSTRTAYMELRSLRSDDTAVYYCARENYYG |
| | | SGAYRGGMDVWGQGTTVTVSSASTKGPSVFPLAPS |
| | | SKSTSGGTAALGCLVKDYFPEPVTVSWNSGALTSG |
| | | VHTFPAVLQSSGLYSLSSVVTVPSSSLGTQTYICNVN |
| | | HKPSNTKVDKKVEPKSCDKTHTCPPCPAPEAAGAPS |
| | | VFLFPPKPKDTLMISRTPEVTCVVVDVSHEDPEVKF |
| | | NWYVDGVEVHNAKTKPREEQYNSTYRVVSVLTVL |
| | | HQDWLNGKEYKCKVSNKALPAPIEKTISKAKGQPR |
| | | EPQVYTLPPSREEMTKNQVSLTCLVKGFYPSDIAVE |
| | | WESNGQPENNYKTTPPVLDSDGSFFLYSKLTVDKSR |
| | | WQQGNVFSCSVMHEALHNHYTQKSLSLSPG |
| 1237 | M4 VH | QLQLQESGPGLVKPSETLSLTCTVSGGSISSRSYYWG |
| | | WIRQPPGKGLEWIGSIYYNGRTYYNPSLKSRVTISVD |
| | | TSKNQFSLKLSSVTAADTAVYYCAREDYGDYGAFD |
| | | IWGQGTMVTVSS |
| 1238 | M5 VH | QVTLKESGPALVKPTQTLTLTCTFSGFSLSTSNMGV |
| | | VWIRQPPGKALEWLAHILWDDREYSNPALKSRLTIS |
| | | KDTSKNQVVLTMINMDPVDTATYYCARMSRNYYG |
| | | SSYVMDYWGQGTLVTVSS |
| 1239 | M6 VH | QVQLVQSGSELKKPGASVKVSCKASGYTFTLYGMN |
| | | WVRQAPGQGLEWMGWINTYTGEPTYADDFKGRFV |
| | | FSLDTSVSTAYLQISSLKAEDTAVYYCAR |
| | | DTAMDYAMAYWGQGTLVTVSS |
| 1240 | M6 VH | QVQLVQSGSELKKPGASVKVSCKASGYTFTLYGMN |
| | | WVKQAPGKGLKWMGWINTYTGEPTYADDFKGRFV |
| | | FSLDTSVSTAYLQISSLKAEDTAVYFCAR |
| | | DTAMDYAMAYWGQGTLVTVSS |
| 1241 | M6 VH | QVQLVQSGSELKKPGASVKVSCKASGYTFTNYGMN |
| | | WVRQAPGQGLEWMGWINTYTGEPTYADDFKGRFV |
| | | FSLDTSVSTAYLQISSLKAEDTAVYYCAR |
| | | DYGKYGDYYAMDYWGQGTLVTVSS |
| 1242 | M6 VH | QVQLVQSGSELKKPGASVKVSCKASGYTFTNYGMN |
| | | WVRQAPGKGLKWMGWINTYTGEPTYADDFKGRFV |
| | | FSLDTSVSTAYLQISSLKAEDTAVYFCAR |
| | | DYGKYGDYYAMDYWGQGTLVTVSS |
| 1243 | M7 VH | QVQLQQPGSVLVRPGASVKVSCKASGYTFTSSWMH |
| | | WAKQRPGQGLEWIGEIHPNSGGTNYNEKFKGKATV |
| | | DTSSSTAYVDLSSLTSEDSAVYYCARGDYYGYVSW |
| | | FAYWGQGTLVTVSS |
| 1244 | M7 VH | QVQLVQSGAEVKKPGASVKVSCKASGYTFTSSWM |
| | | HWARQAPGQGLEWIGEIHPNSGGTNYAQKFQGRAT |
| | | LTVDTSSSTAYMELSRLRSDDTAVYYCARGDYYGY |
| | | VSWFAYWGQGTLVTVSS |
| 1245 | M7 VH | QVQLVQSGAEVKKPGASVKVSCKASGYTFTSSWM |
| | | HWARQAPGQGLEWIGEIHPNSGGTNYAQKFQGRAT |
| | | MTVDTSISTAYMELSRLRSDDTAVYYCARGDYYGY |
| | | VSWFAYWGQGTLVTVSS |
| 1246 | M7 VH | QVQLVQSGAEVKKPGASVKVSCKASGYTFTSSWM |
| | | HWARQAPGQGLEWIGEIHPNSGGTNYAQKFQGRVT |
| | | MTVDTSISTAYMELSRLRSDDTAVYYCARGDYYGY |
| | | VSWFAYWGQGTLVTVSS |
| 1247 | M7 VH | QVQLVQSGAEVKKPGASVKVSCKASGYTFTSSWM |
| | | HWARQAPGQGLEWMGEIHPNSGGTNYAQKFQGRV |
| | | TMTVDTSISTAYMELSRLRSDDTAVYYCARGDYYG |
| | | YVSWFAYWGQGTLVTVSS |
| 1248 | M8 VH | QVQLVQSGAEVKKPGASVKVSCKASGYTFTSYDIN |
| | | WVRQAPGQGLEWMGWLNPNSGNTGYAQKFQGRV |
| | | TMTADRSTSTAYMELSSLRSEDTAVYYCAREVPET |
| | | AAFEYWGQGTLVTVSS |
| 1249 | M8 VH | QVQLVQSGAEVKKPGASVKVSCKASGYTFTSYDIN |
| | | WVRQAPGQGLEWMGWLNPNSGNTGYAQKFQGRV |
| | | TMTADRSTSTAYMELSSLRSEDTAVYYCAREVPET |
| | | AAFEYWGQGTLVTVSS |
| 1250 | M8 VH | QVQLVQSGAEVKKPGASVKVSCKASGYTFTSYDIN |
| | | WVRQAPGQGLEWMGWLNPNSGYTGYAQKFQGRV |
| | | TMTADRSTSTAYMELSSLRSEDTAVYYCAREVPET |
| | | AAFEYWGQGTLVTVSS |
| 1251 | M8 VH | QVQLVQSGAEVKKPGASVKVSCKASGYTFTSYDIN |
| | | WVRQAPGQGLEWMGWLNPNSGNTGYAQKFQGRV |
| | | TMTADRSTSTAYMELSSLRSEDTAVYYCAREVPET |
| | | AAFEYWGQGTLVTVSS |
| 1252 | M8 VH | QVQLVQSGAEVKKPGASVKVSCKASGYTFTSYDIN |
| | | WVRQAPGQGLEWMGWLNPNSGNTGYAQKFQGRV |
| | | TMTADRSTSTAYMELSSLRSEDTAVYYCAREVPET |
| | | AAFEYWGQGTLVTVSS |
| 1253 | M8 VH | QVQLVQSGAEVKKPGASVKVSCKASGYTFTSYDIN |
| | | WVRQAPGQGLEWMGWLNPNSGNTGYAQKFQGRV |
| | | TMTADRSTSTAYMELSSLRSEDTAVYYCAREVPET |
| | | AAFEYWGQGTLVTVSS |
| 1254 | M8 VH | QVQLVQSGAEVKKPGASVKVSCKASGYTFTSYDIN |
| | | WVRQAPGQGLEWMGWLNPNSGNTGYAQKFQGRV |
| | | TMTADRSTSTAYMELSSLRSEDTAVYYCAREVPET |
| | | AAFEYWGQGTLVTVSS |
| 1255 | M8 VH | QVQLVQSGAEVKKPGASVKVSCKASGYTFTSYDIN |
| | | WVRQAPGQGLEWMGWLNPNSGYTGYAQKFQGRV |
| | | TMTADRSTSTAYMELSSLRSEDTAVYYCAREVPET |
| | | AAFEYWGQGTLVTVSS |
| 1256 | M8 VH | QVQLVQSGAEVKKPGASVKVSCKASGYTFTSYDIN |
| | | WVRQAPGQGLEWMGWLNPNSGYTGYAQKFQGRV |
| | | TMTADRSTSTAYMELSSLRSEDTAVYYCAREVPET |
| | | AAFEYWGQGTLVTVSS |
| 1257 | M8 VH | QVQLVQSGAEVKKPGASVKVSCKASGYTFTSYDIN |
| | | WVRQAPGQGLEWMGWLNPNSGYTGYAQKFQGRV |
| | | TMTADRSTSTAYMELSSLRSEDTAVYYCAREVPET |
| | | AAFEYWGQGTLVTVSS |
| 1258 | M8 VH | QVQLVQSGAEVKKPGASVKVSCKASGYTFTSYDIN |
| | | WVRQAPGQGLEWMGWLNPNSGYTGYAQKFQGRV |
| | | TMTADRSTSTAYMELSSLRSEDTAVYYCAREVPET |
| | | AAFEYWGQGTLVTVSS |
| 1259 | M8 VH | QVQLVQSGAEVKKPGASVKVSCKASGYTFTSYDIN |
| | | WVRQAPGQGLEWMGWLNPNSGYTGYAQKFQGRV |
| | | TMTADRSTSTAYMELSSLRSEDTAVYYCAREVPET |
| | | AAFEYWGQGTLVTVSS |
| 1260 | M9 VH | QVQLQESGPGLVKPSETLSLTCTVSGGSISSYFWSWI |
| | | RQPPGKGLEWIGYIYYSGNTKYNPSLKSRVTISIDTS |
| | | KNQFSLKLSSVTAADTAVYYCARETGSYYGFDYWG |
| | | QGTLVTVSS |
| 1261 | M10 VH | QVQLQQWGAGLLKPSETLSLTCAVHGGSFSGYYW |
| | | NWIRQPPGKGLEWIGEINHAGNTNYNPSLKSRVTISL |
| | | DTSKNQFSLTLTSVTAADTAVYYCARGYCRSTTCYF |
| | | DYWGQGTLVTVSS |
| 1262 | M11 VH | EVQLLESGGGLVQPGKSLRLSCAVSGFTFSTYGMN |
| | | WVRQAPGKGLEWVSSISGTGRTTYHADSVQGRFTV |
| | | SRDNSKNILYLQMNSLRADDTAVYFCTKERGDYYY |
| | | GVFDYWGQGTLVTVSS |
| 1263 | M12 VH | QIQLVQSGPELKKPGETVKISCKASGYTFTTYGMSW |
| | | VKQAPGKGLKWMGWMNTYSGVTTYADDFKGRFA |
| | | FSLETSASTAYMQIDNLKNEDTATYFCAREGYVFDD |
| | | YYATDYWGQGTSVTVSS |
| 301 | Variable Heavy 1 | X1VQLVQSGAEVKKPGASVKVSCKAS[HCDR1]WV |
| | | X2QX3PGQGLEWX4G[HCDR2]RX5TX6TX7DTSTST |
| | | X8YX9ELSSLRSEDTAVYYCAR[HCDR3]WGQGTTV |
| | | TVSS |
| | | wherein each of X1-X11 is independently selected from A, |
| | | R, N, D, C, Q, E, G, H, I, L, K, M, F, P, S, T, W, Y, or |
| | | V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 302 | Variable Heavy 2 | X1VQLVQSGAEVKKPGASVKVSCKAS[HCDR1]WV |
| | | X2QX3PGQGLEWX4G[HCDR2]RX5TX6TX7DTSTST |
| | | X8YX9ELSSLRSEDTAVYYC[HCDR3]WGQGTTVTVS |
| | | S |
| | | wherein each of X1-X11 is independently selected from A, |
| | | R, N, D, C, Q, E, G, H, I, L, K, M, F, P, S, T, W, Y, or |
| | | V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 1301 | Variable Heavy 3 | X1VQLVQSGAEVKKPGASVKVSCKAS[HCDR1]WV |
| | | X2QRPGQGLEWX4G[HCDR2]RX5TX6TX7DTSTSTX |
| | | 8YX9ELSSLRSEDTAVYYCAR[HCDR3]WGQGTTVT |
| | | VSS |
| | | wherein each of X1, X2, X4, X5, X6, X7, X8, X9, X10, |
| | | and X11 is independently selected from A, R, N, D, C, Q, |
| | | E, G, H, I, L, K, M, F, P, S, T, W, Y, or V |
| | | In some cases, X1 = Q or E |
| | | In some cases, X2 = R or K |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 1302 | Variable Heavy 4 | X1VQLVQSGAEVKKPGASVKVSCKAS[HCDR1]WV |
| | | X2QRPGQGLEWX4G[HCDR2]RX5TX6TX7DTSTSTX |
| | | 8YX9ELSSLRSEDTAVYYC[HCDR3]WGQGTTVTVSS |
| | | wherein each of X1, X2, X4, X5, X6, X7, X8, X9, X10, |
| | | and X11 is independently selected from A, R, N, D, C, Q, |
| | | E, G, H, I, L, K, M, F, P, S, T, W, Y, or V |
| | | In some cases, X1 = Q or E |
| | | In some cases, X2 = R or K |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |

TABLE 17
Light Chain Variable Region (VL) Amino Acid Sequences

| SEQ | | |
|---|---|---|
| ID | | |
| NO | Description | Sequence |
| 201 | 217 VL, | EIVLTQSPGTLSLSPGERATLSCRASSSVSYMYWYQ |
| | 219 VL, 221 VL, | QKPGQAPRPLIYATSNLASGIPDRFSGSGSGTDFTLTI |
| | 200 VL, 213 VL, | SRLEPEDFAVYYCQQWEGNPRTFGGGTKLEIK |
| | 212 VL, 211 VL, | |
| | 199 VL, 214 VL, | |
| | 216 VL, 222 VL, | |
| | 203 VL, 147 VL, | |
| | 218 VL, 179 VL, | |
| | 148 VL, 149 VL, | |
| | 151 VL, 180 VL, | |
| | 175 VL, 178 VL, | |
| | 145 VL, 146 VL, | |
| | 150 VL, 152 VL, | |
| | 176 VL, 177 VL, | |
| | 201 VL, 202 VL, | |
| | 204 VL, 215 VL, | |
| | 224 VL | |
| 202 | 223 VL, 107 VL, | EIVLTQSPGTLSLSPGERATLSCRASSSVSYMYWYQ |
| | 205 VL, 181 VL, | QKPGQAPRLLIYATSNLASGIPDRFSGSGSGTDFTLTI |
| | 188 VL, 64 VL, | SRLEPEDFAVYYCQQWEGNPRTFGGGTKLEIK |
| | 67 VL, 68 VL, | |
| | 94 VL, 33 VL, 57 | |
| | VL, 58 VL, 59 VL, | |
| | 60 VL, 61 VL, 62 | |
| | VL, 63 VL, 65 VL, | |
| | 66 VL, 69 VL, 70 | |
| | VL, 71 VL, 72 VL, | |
| | 76 VL, 77 VL, 78 | |
| | VL, 91 VL, 92 VL, | |
| | 93 VL, 97 VL, 98 | |
| | VL, 99 VL, 140 | |
| | VL, 142 VL, 143 | |
| | VL, 182 VL, 183 | |
| | VL, 184 VL, 185 | |
| | VL, 186 VL, 187 | |
| | VL, 189 VL, 190 | |
| | VL, 191 VL, 192 | |
| | VL, 206 VL, 207 | |
| | VL, 208 VL, 209 | |
| | VL, 210 VL, 18-7 | |
| | S93E VL, clone 34 | |
| | VL, clone 2 VL, | |
| | clone 46 VL, clone | |
| | 47 VL, clone 23 | |
| | VL, clone 53 | |
| 203 | 15 VL, 18-7, L8, | EIVLTQSPGTLSLSPGERATLSCRASSSVSYMYWYQ |
| | clone L8mod VL, | QKPGQAPRLLIYATSNLASGIPDRFSGSGSGTDFTLTI |
| | clone X-V VL, | SRLEPEDFAVYYCQQWSGNPRTFGGGTKLEIK |
| | clone X VL, clone | |
| | H3-1 VL, clone | |
| | H2-2 VL, clone | |
| | H2-5 VL | |
| 204 | 30 VL, 100 VL, | EIVLTQSPGTLSLSPGERATLSCRASSSVSYMYWYQ |
| | 129 VL, 122 VL, | QKPGQAPRLLIYATSNLASGIPDRFSGSGSGTDFTLTI |
| | 127 VL, 126 VL, | SRLEPEDFAVYYCQQWKGNPRTFGGGTKLEIK |
| | 160 VL, 157 VL, | |
| | 159 VL, 158 VL, | |
| | 125 VL, 103 VL, | |
| | 101 VL, 102 VL, | |
| | 104 VL, 10S VL, | |
| | 121 VL, 123 VL, | |
| | 124 VL, 128 VL, | |
| | 144 VL, 161 VL, | |
| | 162 VL, 163 VL, | |
| | 164 VL, clone L3- | |
| | 13 VL | |
| 205 | 110 VL, 197 VL, | EIVLTQSPGTLSLSPGERATLSCRASSSVSYMYWYQ |
| | 112 VL, 169 VL, | QKPGQAPRPWIYATSNLASGIPDRFSGSGSGTDFTLT |
| | 173 VL, 115 VL, | ISRLEPEDFAVYYCQQWEGNPRTFGGGTKLEIK |
| | 113 VL, 96 VL, | |
| | 196 VL, 172 VL, | |
| | 75 VL, 174 VL, | |
| | 109 VL, 198 VL, | |
| | 170 VL, 29 VL, 31 | |
| | VL, 32 VL, 73 VL, | |
| | 74 VL, 95 VL, 108 | |
| | VL, 111 VL, 114 | |
| | VL, 116 VL, 117 | |
| | VL, 118 VL, 119 | |
| | VL, 120 VL, 130 | |
| | VL, 153 VL, 154 | |
| | VL, 155 VL, 156 | |
| | VL, 165 VL, 166 | |
| | VL, 167 VL, 168 | |
| | VL, 171 VL, 193 | |
| | VL, 194 VL, 195 | |
| | VL, 220 VL | |
| 206 | 134 VL, 132 VL, | EIVLTQSPGTLSLSPGERATLSCRASSSVSYMYWYQ |
| | 133 VL, 135 VL, | QKPGQAPRPLIYATSNLASGIPDRFSGSGSGTDFTLTI |
| | 136 VL | SRLEPEDFAVYYCQQWKGNPRTFGGGTKLEIK |
| 207 | 138 VL, 137 VL, | EIVLTQSPGTLSLSPGERATLSCRASSSVSYMYWYQ |
| | 139 VL, 141 VL, | QKPGQAPRLLIYATSNLASGIPDRFSGSGSGTDFTLTI |
| | clone XL3-15 VL | SRLEPEDFAVYYCQQWSRNPRTFGGGTKLEIK |
| 208 | 34 VL, 35 VL, 36 | EIVLTQSPGTLSASPGERATMSCRASSSVSYMYWYQ |
| | VL, 37 VL, 38 VL, | QKPGQAPRPWIYATSNLASGVPDRFSGSGSGTDYTL |
| | 39 VL, 40 VL, 41 | TISRVEPEDFAVYYCQQWSGNPRTFGGGTKLEIK |
| | VL, 42 VL, 43 VL, | |
| | 44 VL, 45 VL, 46 | |
| | VL, 47 VL, 53 VL, | |
| | 54 VL, 55 VL, 79 | |
| | VL, 81 VL, 82 VL, | |
| | 83 VL, AS12824 | |
| | VL | |
| 209 | 85 VL | EIVLTQSPGTLSLSPGERATLSCRASSSVSYMYWYQ |
| | | QKPGQAPRLLIYATSNLASGVPDRFSGSGSGTDFTLT |
| | | ISRLEPEDFAVYYCQQWEGNPRTFGGGTKLEIK |
| 210 | 48 VL | EIVLTQSPGTLSASPGERATLSCRASSSVSYMYWYQ |
| | | QKPGQAPRPWIYATSNLASGVPDRFSGSGSGTDYTL |
| | | TISRVEPEDFAVYYCQQWSGNPRTFGGGTKLEIK |
| 211 | 49 VL | EIVLTQSPGTLSASPGERATMSCRASSSVSYMYWYQ |
| | | QKPGQAPRLLIYATSNLASGVPDRFSGSGSGTDYTL |
| | | TISRVEPEDFAVYYCQQWSGNPRTFGGGTKLEIK |
| 212 | 50 VL | EIVLTQSPGTLSASPGERATMSCRASSSVSYMYWYQ |
| | | QKPGQAPRPWIYATSNLASGVPDRFSGSGSGTDFTL |
| | | TISRVEPEDFAVYYCQQWSGNPRTFGGGTKLEIK |
| 213 | 51 VL | EIVLTQSPGTLSASPGERATMSCRASSSVSYMYWYQ |
| | | QKPGQAPRPWIYATSNLASGVPDRFSGSGSGTDYTL |
| | | TISRLEPEDFAVYYCQQWSGNPRTFGGGTKLEIK |
| 214 | 52 VL | EIVLTQSPGTLSASPGERATMSCRASSSVSYMYWYQ |
| | | QKPGQAPRPWIYATSNLASGVPDRFSGSGSGTDFTL |
| | | TISRLEPEDFAVYYCQQWSGNPRTFGGGTKLEIK |
| 215 | 56 VL | EIVLTQSPGTLSASPGERATMSCRASSSVSYMYWYQ |
| | | QKPGQAPRPWIYATSNLASGIPDRESGSGSGTDYTLT |
| | | ISRVEPEDFAVYYCQQWSGNPRTFGGGTKLEIK |
| 216 | 86 VL | EIVLTQSPGTLSLSPGERATLSCRASSSVSYMYWYQ |
| | | QKPGQAPRLLIYATSNLASGIPDRFSGSGSGTDYTLT |
| | | ISRLEPEDFAVYYCQQWEGNPRTFGGGTKLEIK |
| 217 | 87 VL | EIVLTQSPGTLSLSPGERATLSCRASSSVSYMYWYQ |
| | | QKPGQAPRLLIYATSNLASGIPDRFSGSGSGTDFTLTI |
| | | SRVEPEDFAVYYCQQWEGNPRTFGGGTKLEIK |
| 218 | 88 VL | EIVLTQSPGTLSLSPGERATLSCRASSSVSYMYWYQ |
| | | QKPGQAPRLLIYATSNLASGIPDRFSGSGSGTDYTLT |
| | | ISRVEPEDFAVYYCQQWEGNPRTFGGGTKLEIK |
| 219 | 89 VL | EIVLTQSPGTLSASPGERATLSCRASSSVSYMYWYQ |
| | | QKPGQAPRLLIYATSNLASGIPDRFSGSGSGTDFTLTI |
| | | SRLEPEDFAVYYCQQWEGNPRTFGGGTKLEIK |
| 220 | 90 VL | EIVLTQSPGTMSLSPGERATLSCRASSSVSYMYWYQ |
| | | QKPGQAPRLLIYATSNLASGIPDRFSGSGSGTDFTLTI |
| | | SRLEPEDFAVYYCQQWEGNPRTFGGGTKLEIK |
| 1264 | 5C3D11 VL | QIVLSQSPAILSASPGEKVTMTCRASSSVSYMYWYQ |
| | | QKPGSSPKPWIYATSNLASGVPDRFSGSGSGTSYSLT |
| | | ISRVEAEDAATYYCQQWSGNPRTFGGGTKLEIK |
| 1265 | 9E12ES VL | MKLPVRLLVLMFWIPASSSDVLMTQTPLSLPVSLGD |
| | | QASISCRSSQTIVHSNGDTYLDWFLQKPGQSPKLLIY |
| | | KVSNRFSGVPDRFSGSGSGTDFTLKISRVEAEDLGV |
| | | YYCFQGSHVPYTFGGGTKLEIK |
| 1266 | AS12823 VL | EIVLTQSPGTLSLSPGERATMSCRASSSVSYMYWYQ |
| | | QKPGQAPRPWIYATSNLASGIPDRFSGSGSGTDFTLT |
| | | ISRVEPEDFAVYYCQQWSGNPRTFGGGTKLEIK |
| 1267 | AS12819 VL | EIVLTQSPGTLSLSPGERVTMSCRASSSVSYMYWYQ |
| | | QKPGQAPRPWIYATSNLASGVPDRFSGSGSGTDFTL |
| | | TISRVEPEDFAVYYCQQWSGNPRTFGGGTKVEIK |
| 1268 | AS12816 VL | EIVLTQSPGTLSASPGERVTLSCRASSSVSYMYWYQ |
| | | QKPGQAPRPWIYATSNLASGVPDRFSGSGSGTDFTL |
| | | TISRLEPEDFAVYYCQQWSGNPRTFGGGTKLEIK |
| 1269 | AS12825 VL | EIVLTQSPGTLSASPGERVTMSCRASSSVSYMYWYQ |
| | | QKPGQAPRLLIYATSNLASGVPDRFSGSGSGTDFTLT |
| | | ISRVEPEDFAVYYCQQWSGNPRTFGGGTKLEIK |
| 1270 | 12835 VL | EIVLTQSPGTLSLSPGERVTMSCRASSSVSYMYWYQ |
| | | QKPGQAPRPWIYATSNLASGVPDRFSGSGSGTDYTL |
| | | TISRLEPEDFAVYYCQQWSGNPRTFGGGTKLEIK |
| 1271 | 21-3 VL | EIVLTQSPGTLSLSPGERATLSCRASSSVSYMYWYQ |
| | | QKPGQAPRLLIYATSNLASGVPDRFSGSGSGTDYTL |
| | | TISRLEPEDFAVYYCQQWSGNPRTFGGGTKLEIK |
| 1272 | 18-7 S92D VL | EIVLTQSPGTLSLSPGERATLSCRASSSVSYMYWYQ |
| | | QKPGQAPRLLIYATSNLASGIPDRFSGSGSGTDFTLTI |
| | | SRLEPEDFAVYYCQQWDGNPRTFGGGTKLEIK |
| 1273 | 18-7 S92H VL | EIVLTQSPGTLSLSPGERATLSCRASSSVSYMYWYQ |
| | | QKPGQAPRLLIYATSNLASGIPDRFSGSGSGTDFTLTI |
| | | SRLEPEDFAVYYCQQWHGNPRTFGGGTKLEIK |
| 1274 | 18-7 S92N VL | EIVLTQSPGTLSLSPGERATLSCRASSSVSYMYWYQ |
| | | QKPGQAPRLLIYATSNLASGIPDRFSGSGSGTDFTLTI |
| | | SRLEPEDFAVYYCQQWNGNPRTFGGGTKLEIK |
| 1275 | 18-7 892Q VL, | EIVLTQSPGTLSLSPGERATLSCRASSSVSYMYWYQ |
| | clone 14 VL, clone | QKPGQAPRLLIYATSNLASGIPDRFSGSGSGTDFTLTI |
| | 16L VL, clone 17L | SRLEPEDFAVYYCQQWQGNPRTFGGGTKLEIK |
| | VL, clone 17L-1 | |
| | VL, clone 3-17L V- | |
| | A VL, clone 3-17L | |
| | VL | |
| 1276 | 18-7 CDRv VL | EIVLTQSPGTLSLSPGERATLSCRASSSVSYMYWYQ |
| | | QKPGQAPRLLIYATSNLASGIPDRFSGSGSGTDFTLTI |
| | | SRLEPEDFAVYYCX₁QWX₂X₃X₄PRTFGGGTKLEIK |
| | | X₁ = Q or N |
| | | X₂ = D, E, H, N, Q, or S |
| | | X₃ = A or G |
| | | X₄ = D, F, K, N, R, S, or T |
| 1277 | Clone 52 VL, clone | EIVLTQSPGTLSLSPGERATLSCGASSSVSYMYWYQ |
| | A1 VL, clone E1 | QKPGQAPRLLIYATSNLASGIPDRFSGSGSGTDFTLTI |
| | VL | SRLEPEDFAVYYCQQWEGNPRTFGGGTKLEIK |
| 1278 | Clone XL3-6 VL | EIVLTQSPGTLSLSPGERATLSCRASSSVSYMYWYQ |
| | | QKPGQAPRLLIYATSNLASGIPDRFSGSGSGTDFTLTI |
| | | SRLEPEDFAVYYCSQWSGNPRTFGGGTKLEIK |
| 1279 | Clone XL3-10 VL | EIVLTQSPGTLSLSPGERATLSCRASSSVSYMYWYQ |
| | | QKPGQAPRLLIYATSNLASGIPDRFSGSGSGTDFTLTI |
| | | SRLEPEDFAVYYCQQWSGNPRSFGGGTKLEIK |
| 1280 | M1 VL | DIVMTQSPDSLAVSLGERATINCKSSQSILYSSNNKN |
| | | YLAWYQQKPGQPPKLLIYWASTRESGVPDRFSGSGS |
| | | GTDFTLTISSLQAEDVS |
| | | VYYCQQYYSTPFTFGPGTKVDIK |
| 1281 | M2 VL | DIMLTQTPLTSPVTLGQPASISCKSSQSLVHSDGNTY |
| | | LSWLQQRPGQPPRLLFYKISNRFSGVPDRFSGSGAG |
| | | TDFTLKISRVEAEDVGVYYCMQATQFPLTFGGGTK |
| | | VEIK |
| 1282 | M3 VL | EIVLTQSPATLSLSPGERATLSCRASQSVSSYLAWYQ |
| | | QKPGQAPRLLIYDASNRATGIPARFSGSGSGTDFTLT |
| | | ISSLEPEDFAVYYCQQRSNWPWTFGQGTKVEIK |
| 1283 | M3 VL | EIVLTQSPATLSLSPGERATLSCRASQSVSSYLAWYQ |
| | | QKPGQAPRLLIYDASNRATGIPARFSGSGSGTDFTLT |
| | | ISSLEPEDFAVYYCQQRSNWPWTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 1284 | M4 VL | AIQLTQSPSSLSASVGDRVTITCRASQGISSALAWYQ |
| | | QKPGKAPKLLIYDASSLESGVPSRFSGSGSGTDFTLTI |
| | | SSLQPEDFATYYCQQFNSYPLTFGGGTKVEIK |
| 1285 | M5 VL | DIQLTQSPSFLSASVGDRVTITCSASSSVNYMHWYQ |
| | | QKPGKAPKLLIYSTSNLASGVPSRFSGSGSGTEFTLTI |
| | | SSLQPEDFATYYCHQWNNYGTFGQGTKVEIKR |
| 1286 | M6 VL | DVVMTQSPLSLPVTLGQPASISCKSSQNIVHSDGNTY |
| | | LEWFQQRPGQSPRRLIYKVSNRFSGVPDRFSGSGSG |
| | | TDFTLKISRVEAEDVGVYYCFQGSHVPLTFGGGTKV |
| | | EIKR |
| 1287 | M6 VL | DVVMTQSPLSLPVTLGQPASISCKSSQNIVHSDGNTY |
| | | LEWFQQRPGQSPRRLIYKVSNRFSGVPDRFSGSGSG |
| | | TDFTLKISRVEAEDVGVYYCFQGSHVPLTFGQGTKV |
| | | EIKR |
| 1288 | M6 VL | DVVMTQTPLSLPVTPGEPASISCKSSQNIVHSDGNTY |
| | | LEWYLQKPGQSPQLLIYKVSNRFSGVPDRFSGSGSG |
| | | TDFTLKISRVEAEDLGVYYCFQGSHVPLTFGGGTKV |
| | | EIKR |
| 1289 | M6 VL | DVVMTQTPLSLPVSLGDQASISCKSSQNIVHSDGNT |
| | | YLEWYLQKPGQSPKVLIYKVSNRFSGVPDRFSGSGS |
| | | GTDFTLKISRVEAEDLGVYYCFQGSHVPLTFGGGTK |
| | | VEIKR |
| 1290 | M6 VL | DVVMTQSPLSLPVTLGQPASISCRSSQSIVHSNGNTY |
| | | LDWFQQRPGQSPRRLIYKVSNRFSGVPDRFSGSGSG |
| | | TDFTLKISRVEAEDVGVYYCFQGSHVPLTFGGGTKV |
| | | EIKR |
| 1291 | M6 VL | DVVMTQSPLSLPVTLGQPASISCRSSQSIVHSNGNTY |
| | | LDWFQQRPGQSPRRLIYKVSNRFSGVPDRFSGSGSG |
| | | TDFTLKISRVEAEDVGVYYCFQGSHVPLTFGQGTKV |
| | | EIKR |
| 1292 | MG VL | DVVMTQTPLSLPVTPGEPASISCRSSQSIVHSNGNTY |
| | | LDWYLQKPGQSPQLLIYKVSNRFSGVPDRFSGSGSG |
| | | TDFTLKISRVEAEDLGVYYCFQGSHVPLTFGGGTKV |
| | | EIKR |
| 1293 | M6 VL | DVVMTQTPLSLPVSLGDQASISCRSSQSIVHSNGNTY |
| | | LDWYLQKPGQSPKVLIYKVSNRFSGVPDRFSGSGSG |
| | | TDFTLKINRVEAEDLGVYFCFQGSHVPLTFGGGTKL |
| | | EIKR |
| 1294 | M7 VL | DIQMNQSPSSLSASLGDTITITCHASQNINVLLSWYQ |
| | | QKPGNIPKLLIYKASNLHTGVPSRFSGSGSGTGFTFTI |
| | | SSLQPEDIATYYCQQGQSYPYTFGGGTKLEIK |
| 1295 | M7 VL | DIQMTQSPSSLSASVGDRVTITCQASQDISNYLNWY |
| | | QQKPGKAPKLLIYDASNLETGVPSRFSGSGSGTDFTF |
| | | TISSLQPEDIATYYCQQYDNLPYTFGQGTKLEIK |
| 1296 | M7 VL | DIQMTQSPSSLSASVGDRVTITCQASQNINVLLNWY |
| | | QQKPGKAPKLLIYKASNLHTGVPSRFSGSGSGTDFT |
| | | FTISSLQPEDIATYYCQQGQSYPYTFGQGTKLEIK |
| 1297 | M7 VL | DIQMNQSPSSLSASVGDRVTITCQASQNINVLLSWY |
| | | QQKPGKAPKLLIYKASNLHTGVPSRFSGSGSGTDFT |
| | | FTISSLQPEDIATYYCQQGQSYPYTFGQGTKLEIK |
| 1298 | M8 VL | QSVLTQPPSVSGAPGQRVTISCTSSSSDIGAXXGVX |
| | | WYQQLPGTAPKLLIEGYYNRPSGVPDRFSGSKSGTS |
| | | ASLTITGLLPEDEGDYYCQSXDGTLSALFGGGTKLT |
| | | VLG |
| 1299 | M8 VL | QSVLTQPPSVSGAPGQRVTISCTSSSSDIGAGLGVHW |
| | | YQQLPGTAPKLLIEGYYNRPSGVPDRFSGSKSGTSAS |
| | | LTITGLLPEDEGDYYCQSWDGTLSALFGGGTKLTVL |
| | | G |
| 1300 | M8 VL | QSVLTQPPSVSGAPGQRVTISCTSSSSDIGAGLGVHW |
| | | YQQLPGTAPKLLIEGYYNRPSGVPDRFSGSKSGTSAS |
| | | LTITGLLPEDEGDYYCQSYDGTLSALFGGGTKLTVL |
| | | G |
| 1304 | M8 VL | QSVLTQPPSVSGAPGQRVTISCTSSSSDIGAALGVHW |
| | | YQQLPGTAPKLLIEGYYNRPSGVPDRFSGSKSGTSAS |
| | | LTITGLLPEDEGDYYCQSWDGTLSALFGGGTKLTVL |
| | | G |
| 1305 | M8 VL | QSVLTQPPSVSGAPGQRVTISCTSSSSDIGAGSGVHW |
| | | YQQLPGTAPKLLIEGYYNRPSGVPDRFSGSKSGTSAS |
| | | LTITGLLPEDEGDYYCQSWDGTLSALFGGGTKLTVL |
| | | G |
| 1306 | M8 VL | QSVLTQPPSVSGAPGQRVTISCTSSSSDIGAGQGVH |
| | | WYQQLPGTAPKLLIEGYYNRPSGVPDRESGSKSGTS |
| | | ASLTITGLLPEDEGDYYCQSWDGTLSALFGGGTKLT |
| | | VLG |
| 1307 | M8 VL | QSVLTQPPSVSGAPGQRVTISCTSSSSDIGAGLGVLW |
| | | YQQLPGTAPKLLIEGYYNRPSGVPDRFSGSKSGTSAS |
| | | LTITGLLPEDEGDYYCQSWDGTLSALFGGGTKLTVL |
| | | G |
| 1308 | M8 VL | QSVLTQPPSVSGAPGQRVTISCTSSSSDIGAGLGVHW |
| | | YQQLPGTAPKLLIEGYYNRPSGVPDRFSGSKSGTSAS |
| | | LTITGLLPEDEGDYYCQSWDGTLSALFGGGTKLTVL |
| | | G |
| 1309 | M8 VL | QSVLTQPPSVSGAPGQRVTISCTSSSSDIGAGSGVHW |
| | | YQQLPGTAPKLLIEGYYNRPSGVPDRFSGSKSGTSAS |
| | | LTITGLLPEDEGDYYCQSWDGTLSALFGGGTKLTVL |
| | | G |
| 1310 | M8 VL | QSVLTQPPSVSGAPGQRVTISCTSSSSDIGAGQGVH |
| | | WYQQLPGTAPKLLIEGYYNRPSGVPDRFSGSKSGTS |
| | | ASLTITGLLPEDEGDYYCQSWDGTLSALFGGGTKLT |
| | | VLG |
| 1311 | M8 VL | QSVLTQPPSVSGAPGQRVTISCTSSSSDIGAGLGVLW |
| | | YQQLPGTAPKLLIEGYYNRPSGVPDRFSGSKSGTSAS |
| | | LTITGLLPEDEGDYYCQSWDGTLSALFGGGTKLTVL |
| | | G |
| 1312 | M8 VL | QSVLTQPPSVSGAPGQRVTISCTSSSSDIGAGLGVHW |
| | | YQQLPGTAPKLLIEGYYNRPSGVPDRFSGSKSGTSAS |
| | | LTITGLLPEDEGDYYCQSFDGTLSALFGGGTKLTVL |
| | | G |
| 1313 | M9 VL | DIQMTQSPSSLSASVGDRVTITCRASQSINNYLNWY |
| | | QQRPGKAPKLLIYAASSLQSGVPSRFSGSGSGTDFTL |
| | | TISSLQPGDFATYYCQQSYSTPRTFGQGTKLEIK |
| 1314 | M10 VL | EIVLTQSPGTLSLSPGERATLSCRASQSVRSSYLAWY |
| | | QQKPGQAPRLLIYGASSRATGIPDRFSGSGSGTDFTL |
| | | TISRLEPEDFAVYYCQQYGSSPTFGQGTRLEIK |
| 1315 | M11 VL | DIQMTQSPSTLSASVGDRVTITCRASQTISSWLAWY |
| | | QQTPEKAPKLLIYAASNLQSGVPSRFSGSGSGTEFTL |
| | | TISSLQPDDFATYYCQQYHRSWTFGQGTKVEIT |
| 1316 | M12 VL | DVLMTQTPLSLPVSLGDQASISCRSSQNIVHSDGNTY |
| | | LEWYLQKPGQSPKLLIYKVSNRFSGVPDRFSGSGSG |
| | | TDFTLKISRVEAEDLGIYYCFQGSHVPLTFGAGTKLE |
| | | LK |
| 303 | Variable Light | ETVLTQSPGTLSLSPGERATLSC[LCDR1]WYQQKPG |
| | | QAPRX10X11IY[LCDR2]GIPDRFSGSGSGTDFTLTISR |
| | | LEPEDFAVYYC[LCDR3]FGGGTKLEIK |
| | | wherein each of X10 and X11 is independently selected |
| | | from A, R, N, D, C, Q, E, G, H, I, L, K, M, F, P, S, |
| | | T, W, Y, or V |
| | | In some cases, X10 = L or P |
| | | In some cases, X11 = L or W |

TABLE 18A
Additional Sequences

| SEQ | | |
|---|---|---|
| ID | | |
| NO | Description | Sequence |
| 304 | 219 HC FR1, | QVQLVQSGAEVKKPGASVKVSCKAS |
| | 212 HC FR1 | |
| 305 | 219 HC FR2 | WVKQRPGQGLEWMG |
| 313 | 212 HC FR2 | WVRQRPGOGLEWIG |
| 1317 | HC FR2 | WVRQAPGQGLEWMG |
| 306 | 219 HC FR3a | RVTITRDTSTSTVYLELSSLRSEDTAVYYCAR |
| 307 | 219 HC FR3b | RVTITRDTSTSTVYLELSSLRSEDTAVYYC |
| 314 | 212 HC FR3a | RATITTDTSTSTAYLELSSLRSEDTAVYYCAR |
| 315 | 212 HC FR3b | RATITTDTSTSTAYLELSSLRSEDTAVYYC |
| 1318 | HC FR3 | RVTMTRDTSTSTVYMELSSLRSEDTAVYYC |
| 1319 | HC FR3 | RATITTDTSASTAYLQLSSLRSEDTAVYYC |
| 1320 | HC FR3 | RVTITRDTSASTVYMELSSLRSEDTAVYYC |
| 1321 | HC FR3 | RVTITRDTSASTAYMELSSLRSEDTAVYYC |
| 1322 | HC FR3 | RVTMTRDTSTSTVYMELSSLRSEDTAVYYCSR |
| 1323 | HC FR3 | RVTMTRDTSTSTVYMELSSLRSEDTAVYYCAR |
| 308 | 219 HC FR4, | WGQGTTVTVSS |
| | 212 HC FR4 | |
| 309 | 219 LC FR1, | EIVLTQSPGTLSLSPGERATLSC |
| | 212 LC FR1 | |
| 310 | 219 LC FR2, | WYQQKPGQAPRPLIY |
| | 212 LC FR2 | |
| 1324 | LC FR2 | WYQQKPGQAPRLLIY |
| 311 | 219 LC FR3, | GIPDRFSGSGSGTDFTLTISRLEPEDFAVYYC |
| | 212 LC FR3 | |
| 1325 | LC FR3 | GVPDRFSGSGSGTDYTLTISRLEPEDFAVYYC |
| 312 | 219 LC FR4, | FGGGTKLEIK |
| | 212 LC FR4 | |
| 316 | IGHV1-46*02 | QVQLVQSGAEVKKPGASVKVSCKASGYTENSYYM |
| | | HWVRQAPGQGLEWMGIINPSGGSTSYAQKFQGRVT |
| | | MTRDTSTSTVYMELSSLRSEDTAVYYCAR |
| 317 | IGKV3-20*01 | EIVLTQSPGTLSLSPGERATLSCRASQSVSSSYLAWY |
| | | QQKPGQAPRLLIYGASSRATGIPDRFSGSGSGTDFTL |
| | | TISRLEPEDFAVYYCQQYGSSP |
| 319 | Light Chain | RTVAAPSVFIFPPSDEQLKSGTASVVCLLNNFYPREA |
| | Constant | KVQWKVDNALQSGNSQESVTEQDSKDSTYSLSSTL |
| | | TLSKADYEKHKVYACEVTHQGLSSPVTKSFNRGEC |

TABLE 18B
Additional sequences

| SEQ | |
|---|---|
| ID | |
| NO | Sequence |
| 3001 | GFDIQDTYMH |
| 3002 | RIDPASGHTKYDPKFQV |
| 3003 | RIEPASGHIKYDPKFQG |
| 3005 | RIEPASGHIKYDPKFQV |
| 3006 | SGGLPDV |
| 3008 | SGGLPDW |
| 3010 | RASSSVSYMY |
| 3011 | ATSNLAS |
| 3012 | QQWEGNPRT |
| 3013 | QQWKGNPRT |
| 3116 | EVQLVQSGAEVKKPGASVKVSCKASGFDIQDTYMHWVKQAPGQGLEW |
| | MGRIEPASGHIKYDPKFQGRVTMTRDTSTSTVYMELSSLRSEDTAVYYC |
| | ARSGGLPDWWGQGTTVTVSS |
| 3121 | QVQLVQSGAEVKKPGASVKVSCKASGFDIQDTYMHWVRQAPGQGLEW |
| | MGRIEPASGHIKYDPKFQVRVTMTRDTSTSTVYMELSSLRSEDTAVYYC |
| | ARSGGLPDWWGQGTTVTVSS |
| 3122 | QVQLVQSGAEVKKPGASVKVSCKASGFDIQDTYMHWVRQAPGQGLEW |
| | MGRIEPASGHIKYDPKFQVRATMTRDTSTSTVYMELSSLRSEDTAVYYC |
| | ARSGGLPDWWGQGTTVTVSS |
| 3123 | QVQLVQSGAEVKKPGASVKVSCKASGFDIQDTYMHWVRQAPGQGLEW |
| | MGRIEPASGHIKYDPKFQVRVTITRDTSTSTVYMELSSLRSEDTAVYYCA |
| | RSGGLPDWWGQGTTVTVSS |
| 3124 | QVQLVQSGAEVKKPGASVKVSCKASGFDIQDTYMHWVRQAPGQGLEW |
| | MGRIEPASGHIKYDPKFQVRATITRDTSTSTVYMELSSLRSEDTAVYYCA |
| | RSGGLPDWWGQGTTVTVSS |
| 3125 | QVQLVQSGAEVKKPGASVKVSCKASGFDIQDTYMHWVKQAPGQGLEW |
| | MGRIEPASGHIKYDPKFQGRVTMTRDTSTSTVYMELSSLRSEDTAVYYC |
| | ARSGGLPDWWGQGTTVTVSS |
| 3128 | QVQLVQSGAEVKKPGASVKVSCKASGFDIQDTYMHWVRQAPGQGLEW |
| | MGRIEPASGHIKYDPKFQGRATMTRDTSTSTVYMELSSLRSEDTAVYYC |
| | ARSGGLPDWWGQGTTVTVSS |
| 3129 | EVQLVQSGAEVKKPGASVKVSCKASGFDIQDTYMHWVRQAPGQGLEW |
| | MGRIEPASGHIKYDPKFQGRATMTRDTSTSTVYMELSSLRSEDTAVYYC |
| | ARSGGLPDWWGQGTTVTVSS |
| 3131 | QVQLVQSGAEVKKPGASVKVSCKASGFDIQDTYMHWVRQAPGQGLEW |
| | MGRIEPASGHIKYDPKFQGRATITTDTSTSTVYMELSSLRSEDTAVYYCA |
| | RSGGLPDWWGQGTTVTVSS |
| 3133 | QVQLVQSGAEVKKPGASVKVSCKASGFDIQDTYMHWVRQAPGQGLEW |
| | MGRIEPASGHIKYDPKFQGRVTMTRDTSTSTAYMELSSLRSEDTAVYYC |
| | ARSGGLPDWWGQGTTVTVSS |
| 3134 | EVQLVQSGAEVKKPGASVKVSCKASGFDIQDTYMHWVRQAPGQGLEW |
| | MGRIEPASGHIKYDPKFQGRVTMTRDTSTSTAYMELSSLRSEDTAVYYC |
| | ARSGGLPDWWGQGTTVTVSS |
| 3136 | QVQLVQSGAEVKKPGASVKVSCKASGFDIQDTYMHWVRQAPGQGLEW |
| | MGRIDPASGHIKYDPKFQGRVTMTRDTSTSTAYMELSSLRSEDTAVYYC |
| | ARSGGLPDWWGQGTTVTVSS |
| 3137 | DVQLVQSGAEVKKPGASVKVSCKASGFDIQDTYMHWVRQAPGQGLEW |
| | MGRIEPASGHIKYDPKFQGRVTMTRDTSTSTVYMELSSLRSEDTAVYYC |
| | ARSGGLPDWWGQGTTVTVSS |
| 3138 | QVQLVQSGAEVKKPGASVKVSCKASGFDIQDTYMHWVKQAPGQGLEWI |
| | GRIDPASGHTKYDPKFQVRATITTDTSTSTAYLELSSLRSEDTAVYYCARS |
| | GGLPDVWGQGTTVTVSS |
| 3139 | QVQLVQSGAEVKKPGASVKVSCKASGFDIQDTYMHWVRQAPGQGLEWI |
| | GRIDPASGHTKYDPKFQVRATITTDTSTSTAYLELSSLRSEDTAVYYCARS |
| | GGLPDVWGQGTTVTVSS |
| 3151 | QVQLVQSGAEVKKPGASVKVSCKASGFDIQDTYMHWVRQAPGQGLEW |
| | MGRIDPASGHIKYDPKFQGRVTMTRDTSTSTVYMELSSLRSEDTAVYYC |
| | ARSGGLPDWWGQGTTVTVSS |
| 3152 | QVQLVQSGAEVKKPGASVKVSCKASGFDIQDTYMHWVRQAPGQGLEWI |
| | GRIEPASGHIKYDPKFQGRVTMTRDTSTSTVYMELSSLRSEDTAVYYCAR |
| | SGGLPDWWGQGTTVTVSS |
| 3153 | QVQLVQSGAEVKKPGASVKVSCKASGFDIQDTYMHWVRQAPGQGLEWI |
| | GRIDPASGHIKYDPKFQGRVTMTRDTSTSTVYMELSSLRSEDTAVYYCAR |
| | SGGLPDWWGQGTTVTVSS |
| 3154 | QVQLVQSGAEVKKPGASVKVSCKASGFDIQDTYMHWVRQAPGQGLEW |
| | MGRIEPASGHVKYDPKFQGRVTMTRDTSTSTVYMELSSLRSEDTAVYYC |
| | ARSGGLPDWWGQGTTVTVSS |
| 3155 | QVQLVQSGAEVKKPGASVKVSCKASGFDIQDTYMHWVRQAPGQGLEW |
| | MGRIEPASGHIKYDPKFQGRVTITRDTSTSTVYMELSSLRSEDTAVYYCA |
| | RSGGLPDWWGQGTTVTVSS |
| 3156 | EVQLVQSGAEVKKPGASVKVSCKASGFDIQDTYMHWVRQAPGQGLEW |
| | MGRIEPASGHIKYDPKFQGRVTMTRDTSTSTVYMELSSLRSEDTAVYYC |
| | ARSGGLPDWWGQGTTVTVSS |
| 3157 | QVQLVQSGAEVKKPGASVKVSCKASGFDIQDTYMHWVRQAPGQGLEW |
| | MGRIEPASGHIKYDPKFQGRVTMTTDTSTSTVYMELSSLRSEDTAVYYC |
| | ARSGGLPDWWGQGTTVTVSS |
| 3158 | QVQLVQSGAEVKKPGASVKVSCKASGFDIQDTYMHWVRQAPGQGLEW |
| | MGRIEPASGHIKYDPKFQGRVTITTDTSTSTVYMELSSLRSEDTAVYYCA |
| | RSGGLPDWWGQGTTVTVSS |
| 3159 | QVQLVQSGAEVKKPGASVKVSCKASGFDIQDTYMHWVRQAPGQGLEW |
| | MGRIEPASGHTKYDPKFQGRVTMTRDTSTSTVYMELSSLRSEDTAVYYC |
| | ARSGGLPDWWGQGTTVTVSS |
| 3160 | QVQLVQSGAEVKKPGASVKVSCKASGFDIQDTYMHWVRQAPGQGLEW |
| | MGRIEPASGHIKYDPKFQGRATITRDTSTSTVYMELSSLRSEDTAVYYCA |
| | RSGGLPDWWGQGTTVTVSS |
| 3161 | QVQLVQSGAEVKKPGASVKVSCKASGFDIQDTYMHWVRQAPGQGLEW |
| | MGRIEPASGHIKYDPKFQGRVTMTRDTSTSTVYLELSSLRSEDTAVYYCA |
| | RSGGLPDWWGQGTTVTVSS |
| 3162 | QVQLVQSGAEVKKPGASVKVSCKASGFDIQDTYMHWVRQAPGQGLEW |
| | MGRIEPASGHIKYDPKFQGRVTMTRDTSTSTAYLELSSLRSEDTAVYYCA |
| | RSGGLPDWWGQGTTVTVSS |
| 3168 | QVQLVQSGAEVKKPGASVKVSCKASGFDIQDTYMHWVRQAPGQGLEW |
| | MGRIDPASGHIKYDPKFQGRATITTDTSTSTVYMELSSLRSEDTAVYYCA |
| | RSGGLPDWWGQGTTVTVSS |
| 3169 | EVQLVQSGAEVKKPGASVKVSCKASGFDIQDTYMHWVRQAPGQGLEW |
| | MGRIEPASGHIKYDPKFQGRATITTDTSTSTVYMELSSLRSEDTAVYYCA |
| | RSGGLPDWWGQGTTVTVSS |
| 3202 | EIVLTQSPGTLSLSPGERATLSCRASSSVSYMYWYQQKPGQAPRLLIYATS |
| | NLASGIPDRFSGSGSGTDFTLTISRLEPEDFAVYYCQQWEGNPRTFGGGT |
| | KLEIK |
| 3204 | EIVLTQSPGTLSLSPGERATLSCRASSSVSYMYWYQQKPGQAPRLLIYATS |
| | NLASGIPDRFSGSGSGTDFTLTISRLEPEDFAVYYCQQWKGNPRTFGGGT |
| | KLEIK |
| 3205 | EIVLTQSPGTLSLSPGERATLSCRASSSVSYMYWYQQKPGQAPRPWIYAT |
| | SNLASGIPDRFSGSGSGTDFTLTISRLEPEDFAVYYCQQWEGNPRTFGGGT |
| | KLEIK |
| 3206 | EIVLTQSPGTLSLSPGERATLSCRASSSVSYMYWYQQKPGQAPRPLIYATS |
| | NLASGIPDRFSGSGSGTDFTLTISRLEPEDFAVYYCQQWKGNPRTFGGGT |
| | KLEIK |
| 3207 | EIVLTQSPGTLSLSPGERATLSCRASSSVSYMYWYQQKPGQAPRLLIYATS |
| | NLASGIPDRFSGSGSGTDFTLTISRLEPEDFAVYYCQQWSRNPRTFGGGTK |
| | LEIK |
| 3208 | EIVLTQSPGTLSASPGERATMSCRASSSVSYMYWYQQKPGQAPRPWIYA |
| | TSNLASGVPDRFSGSGSGTDYTLTISRVEPEDFAVYYCQQWSGNPRTFGG |
| | GTKLEIK |
| 3209 | EIVLTQSPGTLSLSPGERATLSCRASSSVSYMYWYQQKPGQAPRLLIYATS |
| | NLASGVPDRFSGSGSGTDFTLTISRLEPEDFAVYYCQQWEGNPRTFGGGT |
| | KLEIK |
| 3216 | EIVLTQSPGTLSLSPGERATLSCRASSSVSYMYWYQQKPGQAPRLLIYATS |
| | NLASGIPDRFSGSGSGTDYTLTISRLEPEDFAVYYCQQWEGNPRTFGGGT |
| | KLEIK |
| 3217 | EIVLTQSPGTLSLSPGERATLSCRASSSVSYMYWYQQKPGQAPRLLIYATS |
| | NLASGIPDRFSGSGSGTDFTLTISRVEPEDFAVYYCQQWEGNPRTFGGGT |
| | KLEIK |
| 3218 | EIVLTQSPGTLSLSPGERATLSCRASSSVSYMYWYQQKPGQAPRLLIYATS |
| | NLASGIPDRFSGSGSGTDYTLTISRVEPEDFAVYYCQQWEGNPRTFGGGT |
| | KLEIK |
| 3219 | EIVLTQSPGTLSASPGERATLSCRASSSVSYMYWYQQKPGQAPRLLIYAT |
| | SNLASGIPDRFSGSGSGTDFTLTISRLEPEDFAVYYCQQWEGNPRTFGGGT |
| | KLEIK |
| 3220 | EIVLTQSPGTMSLSPGERATLSCRASSSVSYMYWYQQKPGQAPRLLIYAT |
| | SNLASGIPDRFSGSGSGTDFTLTISRLEPEDFAVYYCQQWEGNPRTFGGGT |
| | KLEIK |
| 3319 | RTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVT |
| | KSFNRGEC |
| 3320 | ASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLN |
| | GKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKS |
| | RWQQGNVFSCSVMHEALHNHYTQKSLSLSPGK |

TABLE 19
Fc and Constant Regions

SEQ ID NO: 320 IgG1 Constant
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 321 IgG1 Constant
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 322 IgG1 Constant
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 323 Fc1 (L235E)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHBALHNHYTQKSLSLSPGK
SEQ ID NO: 324 Fc2 (L235E)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 325 Fc3 (L235E)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 326 Fc4 (L234A, L235A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 327 Fc5 (L234A, L235A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 328 Fc6 (L234A, L235A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 329 Fc7 (L234A, L235A, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 330 Fc8 (L234A, L235A, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 331 Fc9 (L234A, L235A, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 332 Fc10 (L234A, L235A, P329G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALGAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 333 Fc11 (L234A, L235A, P329G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALGAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 334 Fc12 (L234A, L235A, P329G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALGAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 335 Fc13 (L234F, L235E, P331S)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEFEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 336 Fc14 (L234F, L235E, P331S)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEFEGGPSVFLFPPKPKDTLMISRTPEVTCVVVDVS
HEDPEVKENWYVDGVEVHNAKTKPREEQYNSTYRVVSVLTVLHQDWLNGK
EYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 337 Fc15 (L234F, L235E, P331S)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEFEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 338 Fc16 (L234A, L235E, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 339 Fc17 (L234A, L235E, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAEGAPSVFLFPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 340 Fc18 (L234A, L235E, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 341 Fc19 (L234A, L23SE, G237A, P331S)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHBALHNHYTQKSLSLSPG
SEQ ID NO: 342 Fc20 (L234A, L235E, G237A, P331S)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 343 Fc21 (L234A, L235E, G237A, P33IS)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 344 Fc22 (L234A, L235A, P329A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 345 Fc23 (L234A, L235A, P329A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 346 Fc24 (L234A, L235A, P329A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 347 Fc25 (D265A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVAVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 348 Fc26 (D265A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVAVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 349 Fc27 (D265A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVAVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 350 Fc28 (N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYGSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 351 Fc29 (N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYGSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 352 Fc30 (N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYGSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 353 Fc31 (D265A, N297A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVAVS
HEDPEVKFNWYVDGVEVHNAKTKPREEQY ASTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK
SEQ ID NO: 354 Fc32 (D265A, N297A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVAVS
HEDPEVKFNWYVDGVEVHNAKTKPREEQY ASTYRVVSVLTVLHQDWLNG
KEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGK
SEQ ID NO: 355 Fc33 (D265A, N297A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVAVS
HEDPEVKENWYVDGVEVHNAKTKPREEQYASTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 356 Fc34 (D265A, N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVAVS
HEDPEVKFNWYVDGVEVHNAKTKPREEQYGSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 357 Fc35 (D265A, N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVAVS
HEDPEVKFNWYVDGVEVHNAKTKPREEQYGSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 358 Fc36 (D265A, N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVAVS
HEDPEVKENWYVDGVEVHNAKTKPREEQYGSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 359 Fc37 (L235A, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHBALHNHYTQKSLSLSPGK
SEQ ID NO: 360 Fc38 (L235A, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 361 Fc39 (L235A, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 362 Fc40 (IgG4)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
SEQ ID NO: 401 (L234A, L235A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 402 (L235E)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 403 (L234A, L235A, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 404 (L234A, L235E, G237A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 405 (L234A, L235A, P329A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 406 (L234A, L235A, P329G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 407 (P329A)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 408 (L234E, L235F. P331S)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEEFGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 409 (D265A, N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVAVS
HEDPEVKFNWYVDGVEVHNAKTKPREEQYGSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHBALHNHYTQKSLSLSPGK
SEQ ID NO: 410 (N297G)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYGSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 411 (S228P)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
SEQ ID NO: 412 (S228P, L235E)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
SEQ ID NO: 413 (S228P, F234A, L235A)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEAAGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
SEQ ID NO: 501 (L234A, L235A)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 502 (L235B)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELEGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 503 (L234A, L235A, G237A)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGAPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 504 (L234A, L235E, G237A)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAEGAPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 505 (L234A, L235A, P329A)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALAAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 506 (L234A, L235A, P329G)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALAAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 507 (P329A)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALAAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 508 (L234E, L235F, P331S)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEEFGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPASIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 509 (D265A, N297G)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVAVSHEDPEVKFNWYVDGVEVHNAKTKPREEQYGSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 510 (N297G)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYGSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 511 (S228P)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
SEQ ID NO: 512 (S228P, L235E)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFEGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
SEQ ID NO: 513 (S228P, F234A, L235A)
QVQLVQSGAEVKKPGASVKVSCKASGFDIQDTYMHWVKQRPGQGLEWMGR
IDPASGHTKYDPKFQVRVTITRDTSTSTVYLELSSLRSEDTAVYYCARSG
GLPDVWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEAAGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
SEQ ID NO: 514
EIVLTQSPGTLSLSPGERATLSCRASSSVSYMYWYQQKPGQAPRPLIYAT
SNLASGIPDRFSGSGSGTDFTLTISRLEPEDFAVYYCQQWEGNPRTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 515 G2 constant domain
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK

TABLE 20
Select Antibodies

| | Heavy Chain CDR | Light Chain CDR |
|---|---|---|
| | SEQ ID NOS | SEQ ID NOS |
| Antibody | (CDR1, CDR2, CDR3) | (CDR1, CDR2, CDR3) |
| A | 1, 2, 6 | 10, 11, 12 |
| B | 1, 3, 8 | 10, 11, 12 |
| C | 1, 4, 8 | 10, 11, 12 |
| D | 1, 2, 6 | 10, 11, 13 |
| E | 1, 2, 6 | 10, 11, 14 |
| F | 1, 5, 8 | 10, 11, 12 |
| G | 1, 5, 8 | 10, 11, 13 |
| H | 1, 3, 8 | 10, 11, 13 |
| A2 | 1, 2, 7 | 10, 11, 12 |
| B2 | 1, 3, 9 | 10, 11, 12 |
| C2 | 1, 4, 9 | 10, 11, 12 |
| D2 | 1, 2, 7 | 10, 11, 13 |
| E2 | 1, 2, 7 | 10, 11, 14 |
| F2 | 1, 5, 9 | 10, 11, 12 |
| G2 | 1, 5, 9 | 10, 11, 13 |
| H2 | 1, 3, 9 | 10, 11, 13 |
| I | 1, 5, 8 | 10, 11, 15 |
| I2 | 1, 5, 9 | 10, 11, 15 |

TABLE 21
Select Antibodies - Variable Regions

| | Heavy Chain | Light Chain |
|---|---|---|
| | Variable Region | Variable Region |
| Antibody | SEQ ID NO | SEQ ID NOS |
| 15 | 108 | 203 |
| 30 | 108 | 204 |
| 64 | 121 | 202 |
| 67 | 122 | 202 |
| 68 | 123 | 202 |
| 75 | 131 | 205 |
| 94 | 124 | 202 |
| 96 | 128 | 205 |
| 100 | 107 | 204 |
| 103 | 120 | 204 |
| 107 | 108 | 202 |
| 109 | 133 | 205 |
| 110 | 125 | 205 |
| 112 | 117 | 205 |
| 113 | 124 | 205 |
| 115 | 122 | 205 |
| 122 | 113 | 204 |
| 125 | 105 | 204 |
| 126 | 114 | 204 |
| 127 | 118 | 204 |
| 129 | 110 | 204 |
| 132 | 128 | 206 |
| 134 | 122 | 206 |
| 138 | 122 | 204 |
| 147 | 117 | 201 |
| 148 | 121 | 201 |
| 149 | 122 | 201 |
| 151 | 124 | 201 |
| 157 | 104 | 204 |
| 158 | 101 | 204 |
| 159 | 119 | 204 |
| 160 | 102 | 204 |
| 169 | 126 | 205 |
| 170 | 135 | 205 |
| 172 | 130 | 205 |
| 173 | 127 | 205 |
| 174 | 132 | 205 |
| 175 | 126 | 201 |
| 178 | 130 | 201 |
| 179 | 127 | 201 |
| 180 | 132 | 201 |
| 181 | 107 | 202 |
| 188 | 115 | 202 |
| 196 | 129 | 205 |
| 197 | 116 | 205 |
| 198 | 134 | 205 |
| 199 | 109 | 201 |
| 200 | 103 | 201 |
| 203 | 116 | 201 |
| 205 | 109 | 202 |
| 211 | 108 | 201 |
| 212 | 107 | 201 |
| 213 | 106 | 201 |
| 214 | 111 | 201 |
| 216 | 112 | 201 |
| 217 | 101 | 201 |
| 218 | 119 | 201 |
| 219 | 104 | 201 |
| 220 | 102 | 201 |
| 221 | 105 | 201 |
| 222 | 114 | 201 |
| 223 | 103 | 202 |
| 500 | 301 | 303 |
| 501 | 302 | 303 |

### 7.6 Example 6: Design of Humanized Anti-TL1A Antibodies with Reduced Cell-Mediated Cytotoxicity

As provided and described herein, Fc variants (e.g. SEQ ID NOs: 401-413) were designed to diminish effector function and subsequently tested for the ability to (i) effectively be purified/manufactured (Table 22), (ii) reduce antibody-dependent cell-mediated cytotoxicity (ADCC), and (iii) reduce complement-dependent cytotoxicity. Test articles tested comprise heavy chain SEQ ID NOs: 501-513, comprising Fc regions that comprises SEQ ID NOs: 401-413, respectively. Heavy chains used were paired with a light chain comprising SEQ ID NO: 514. ELISA titration profiles and BC50s were generated against recombinant TL1A antigen (“EC50”, Table 23). Interestingly, Fc mutations did affect purity, as measured by monomer content, for select mutations/Fc variants (Table 22, wild-type IgG1 control).

### Reduction of CDC Activity

Test articles were evaluated for CDC activity, compared to negative control Human IgG4 isotype control, on TL1A-expressing HEK293 target cells. Rituxan (anti-CD20) was used as a positive technical control on CD20-expressing Raji cell. All test articles were used at a final top concentration of 10 μg/mL followed by a five-fold dilution series (7 points total), in addition to a no treatment control, in triplicate. Cells were incubated with test articles for 15 minutes at 37 C, then treated with human complement, at a final concentration of 25%, for 3 hours at 37 C, 5% CO2. Following incubation, cells were washed and resuspended in Propidium Iodide (P.I.) at a final concentration of 5 μg/mL prior to flow cytometry analysis. Total cells were examined by flow cytometry during sample acquisition. Data were plotted on an XY chart, graphing percentage P.I. positive cells against the log of the concentration and fit to a non-linear regression curve. Cell cytotoxicity in the presence of all test articles was not distinguishable from cell cytotoxicity in the presence of isotype control (Table 23). CDC bioactivity was observed on Raji target cells with Rituxan treatment.

### Reduction of CDC Activity

An antibody-dependent cell-mediated cytotoxicity (ADCC) reporter assay was performed for the characterization of test articles and IgG4 Isotype control on HEK 293 TL1A cells. A reporter cell line engineered to express human Fc-gamma-RIIIa V158 (high affinity) served as effector cells.

Prometheus test articles were evaluated with a top concentration of 10 μg/mL (log dilution for 7 points total, in addition to no test article control). Treatment conditions were tested in triplicate, effector and target cells were co-cultured for 6 hours at 37 C with 5% CO2. Raji target cells were used as a positive control, with Rituxan treatment at a top concentration of 10 μg/mL. 7-point log dilution series, and no treatment control. Test article 502 treatment resulted in dose-dependent increase in luciferase reporter gene activity, and 5044 treatment resulted in increase of reporter activity at the highest tested concentration. The rest of the test articles did not induce reporter activity (Table 23).

TABLE 22

| | Fc | Heavy | | | Purity | |
|---|---|---|---|---|---|---|
| | SEQ | Chain | | | SDS- | SEC- |
| Class | ID NO | SEQ ID NO | mg/mL | mg | PAGE | HPLC |
| IgG1, | 401 | 501 | 2.65 | 10.60 | 95% | 90% |
| protein | 402 | 502 | 1.15 | 12.65 | 95% | 92% |
| variants | 403 | 503 | 3.22 | 10.62 | 90% | 89% |
| | 404 | 504 | 1.61 | 11.27 | 95% | 92% |
| | 405 | 505 | 3.43 | 10.29 | 95% | 91% |
| | 406 | 506 | 1.51 | 15.10 | 95% | 93% |
| | 407 | 507 | 2.85 | 11.40 | 95% | 92% |
| | 408 | 508 | 1.55 | 10.85 | 95% | 92% |
| IgG1, glycan | 409 | 509 | 2.33 | 9.32 | 90% | 90% |
| knock-out | 410 | 510 | 1.36 | 12.24 | 95% | 92% |
| IgG4 | 411 | 511 | 1.78 | 19.58 | 95% | 82% |
| | 412 | 512 | 2.33 | 18.64 | 90% | 81% |
| | 413 | 513 | 5.08 | 15.24 | 95% | 90% |
| Control | — | — | 3.70 | 5.55 | 95% | 97% |

TABLE 23

| | Fc SEQ | Heavy Chain | EC50 | | |
|---|---|---|---|---|---|
| Class | ID NO | SEQ ID NO | (RM) | ADCC | CDC |
| IgG1, protein | 401 | 501 | 0.222 | ND | ND |
| variants | 402 | 502 | 0.215 | 100 ng/mL | ND |
| | 403 | 503 | 0.188 | ND | ND |
| | 404 | 504 | 0.220 | 10 ug/mL | ND |
| | 405 | 505 | 0.346 | ND | ND |
| | 406 | 506 | 0.347 | ND | ND |
| | 407 | 507 | 0.329 | ND | ND |
| | 408 | 508 | 0.330 | ND | ND |
| IgG1, glycan | 409 | 509 | 0.340 | ND | ND |
| knock-out | 410 | 510 | 0.293 | ND | ND |
| IgG4 | 411 | 511 | 0.299 | ND | ND |
| | 412 | 512 | 0.324 | ND | ND |
| | 413 | 513 | 0.252 | ND | ND |

### 7.7 Example 7: Biophysical Properties of Anti-Tl1a Antibodies at High Concentrations

The data for A219 anti-TL1A antibody properties in solution were analyzed together using a chemometric method termed partial least squares (PLS). Detailed descriptions of PLS modeling have been published in, for example, Katz, M. H. Multivariate Analysis: A Practice Guide for Clinicians. Cambridge University Press, New York, pp. 158-162 (1999); Stahle, L., Wold, K., Multivariate data analysis and experimental design in biomedical research. Prog. Med. Chem. 1988, 25:291-338; Wold S. PLS-regression: a basic tool of chemometrics. Chemom. Intell. Lab. Syst. 2001, 58:109-130; and Martens, H.; Martens, M. Multivariate Analysis of Quality: An Introduction, Wiley and Sons, Chichester, UK (2001).

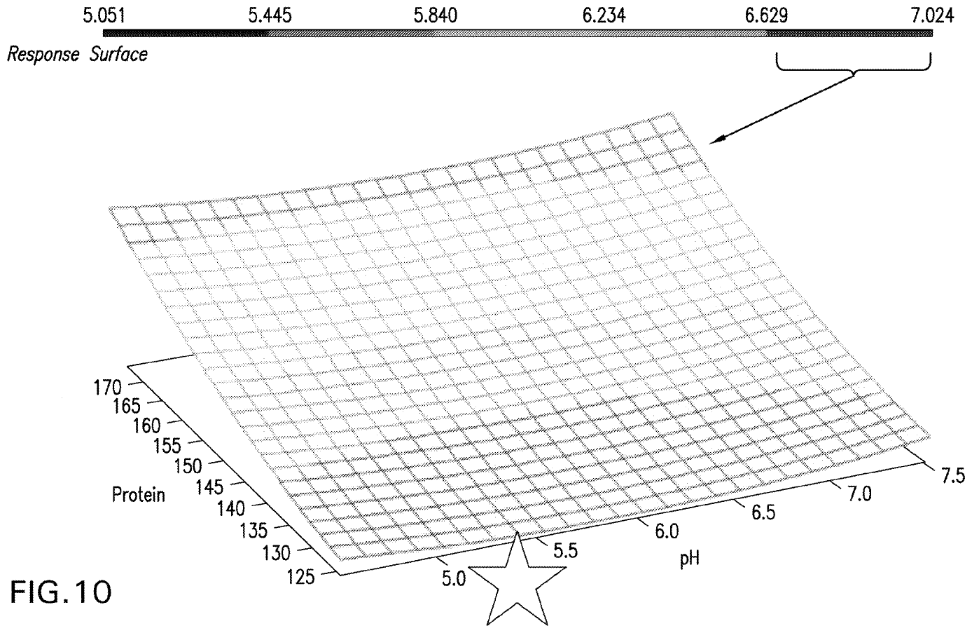

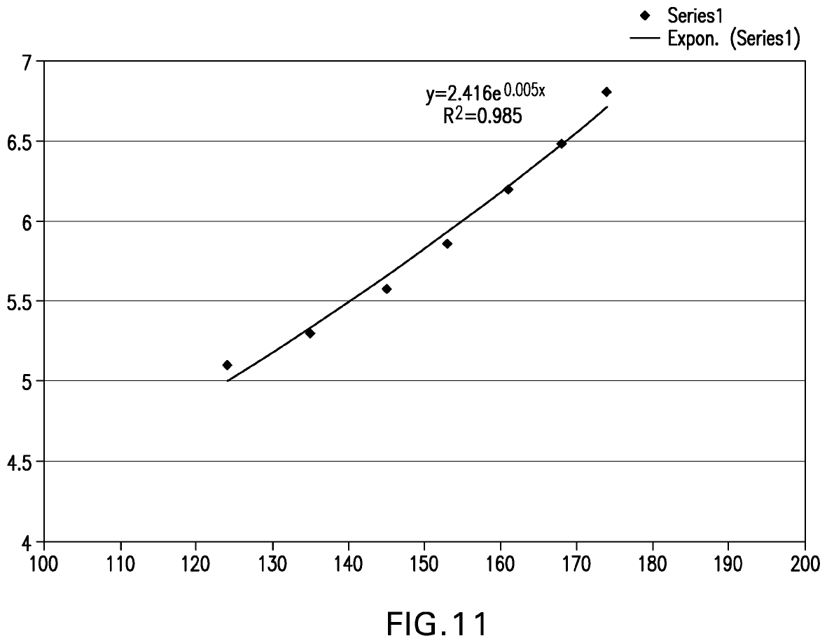

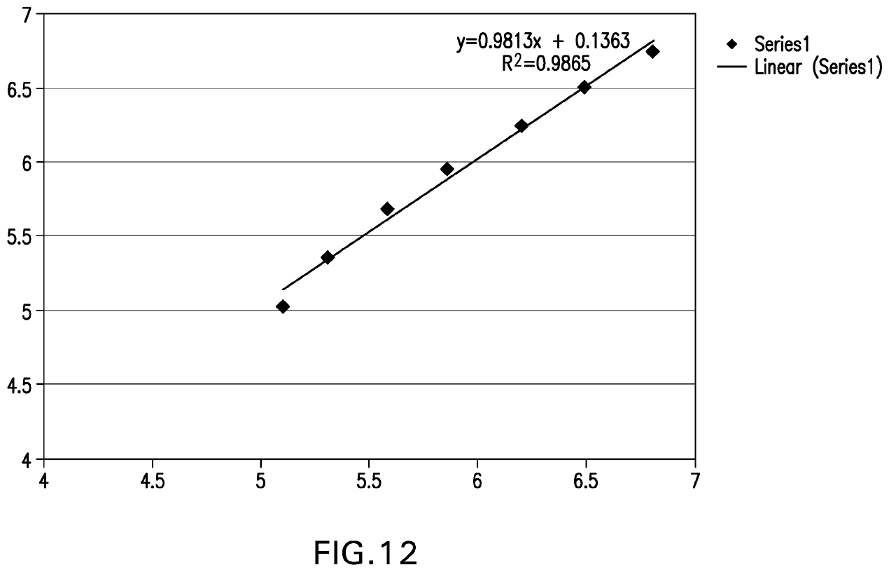

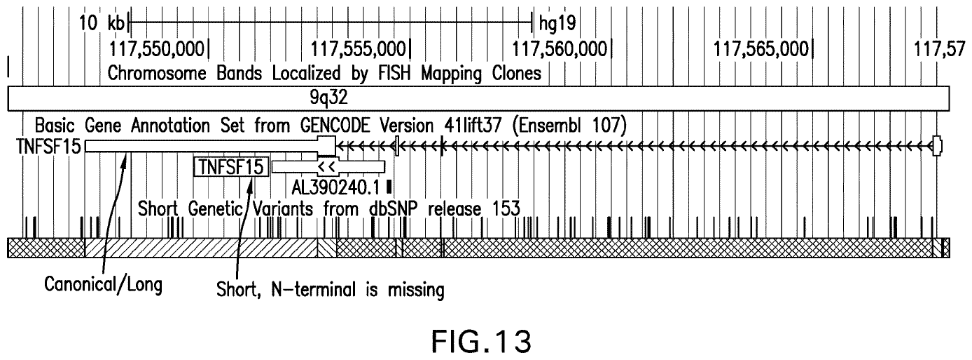

FIGS. 10-12 demonstrate viscosity as a function of antibody concentration and pH. Antibody concentration ranged from greater than about 125 mg/mL to greater than about 170 mg/mL. pH ranged from less than 5.0 to about 7.5. Concentration dependence is evident, with very low viscosities (e.g. as indicated by a viscosity less than 5 mPa-s or 7 mPa-s). The viscosity was measured using an m-VROC™ viscometer by Rheosense with an A10 chip. The shear rates employed were about 1820 s-1. The viscometer was temperature controlled using a ThermoCube thermoelectric chiller and the samples were delivered using a Hamilton 100 μL syringe (81060). The accuracy of the instrument was verified using neat Isopropyl alcohol and measured at 25° C. Furthermore, across the concentration range tested, the percent increase in the HMW fraction as measured by size exclusion chromatography ranged from 0% to a 1.3% increase. HMW as used herein refers to high molecular weight antibody fraction, e.g., aggregated protein, and which excludes monomeric antibody.

TABLE 24
Additional Sequences

| SEQ ID | Sequence |
|---|---|
| 2050 | MAEDLGLSFGETASVEMLPEHGSCRPKARSSSARW |
| >sp\|095150\|TNF15 HUMAN | ALTCCLVLLPFLAGLTTYLLVSQLRAQGEACVQFQ |
| Tumor necrosis factor ligand | ALKGQEFAPSHQQVYAPLRADGDKPRAHLTVVRQT |
| superfamily member 15 | PTQHFKNQFPALHWEHELGLAFTKNRMNYTNKFLL |
| OS = Homo sapiens OX = 9606 | IPESGDYFIYSQVTFRGMTSECSEIRQAGRPNKPD |
| GN = TNFSF15 PE = 1 SV = 2 | SITVVITKVTDSYPEPTQLLMGTKSVCEVGSNWFQ |
| | PIYLGAMFSLQEGDKLMVNVSDISLVDYTKEDKTF |
| | FGAFLL |
| 2051 | MQLTKGRLHFSHPLSHTKHISPFVTDAPLRADGDKP |
| >sp\|095150-2\|TNF15_HUMAN | RAHLTVVRQTPTQHFKNQFPALHWEHELGLAFTKNR |
| Isoform 2 of Tumor necrosis | MNYTNKFLLIPESGDYFIYSQVTFRGMTSECSEIRQ |
| factor ligand superfamily member | AGRPNKPDSITVVITKVTDSYPEPTQLLMGTKSVCE |
| 15 OS = Homo sapiens OX = 9606 | VGSNWFQPIYLGAMFSLQEGDKLMVNVSDISLVDY |
| GN = TNFSF15 | TKEDKTFFGAFLL |
| 2052 | MRRFLSKVYSFPMRKLILFLVFPVVRQTPTQHFKNQ |
| >sp\|095150-3\|TNF15_HUMAN | FPALHWEHELGLAFTKNRMNYTNKFLLIPESGDYFI |
| Isoform 3 of Tumor necrosis | YSQVTFRGMTSECSEIRQAGRPNKPDSITVVITKVT |
| factor ligand superfamily member | DSYPEPTQLLMGTKSVCEVGSNWFQPIYLGAMFSLQ |
| 15 OS = Homo sapiens OX = 9606 | EGDKLMVNVSDISLVDYTKEDKTFFGAFLL |
| GN = TNFSF15 | |

### 7.8 Example 8: A TL1A Expression Assay

This assay is used to determine TL1A gene expression. RNA from a sample derived from a subject is prepared. The prepared RNA is further processed according to RNAseq service provider's instruction (ILLUMINA) and then used to generated cDNA library. Adapters that contain functional elements permitting sequencing are added to the end of the cDNA. The cDNA is amplified, further processed and quality checked according to RNAseq service provider's instruction (ILLUMINA), and analyzed by next generation sequencing, producing sequences that correspond to all or part of the fragment from which it was derived. The number of TL1A isoform transcripts detected by the RNAseq reflects the mRNA level, thus the gene expression level, of the TL1A isoforms.

### 7.9 Example 9: A Phase 2, Multi-Center, Double-Blind, Placebo-Controlled Study to Evaluate the CDx for Treatment with an Anti-TL1A Antibody in Subjects with Moderately to Severely Active Ulcerative Colitis

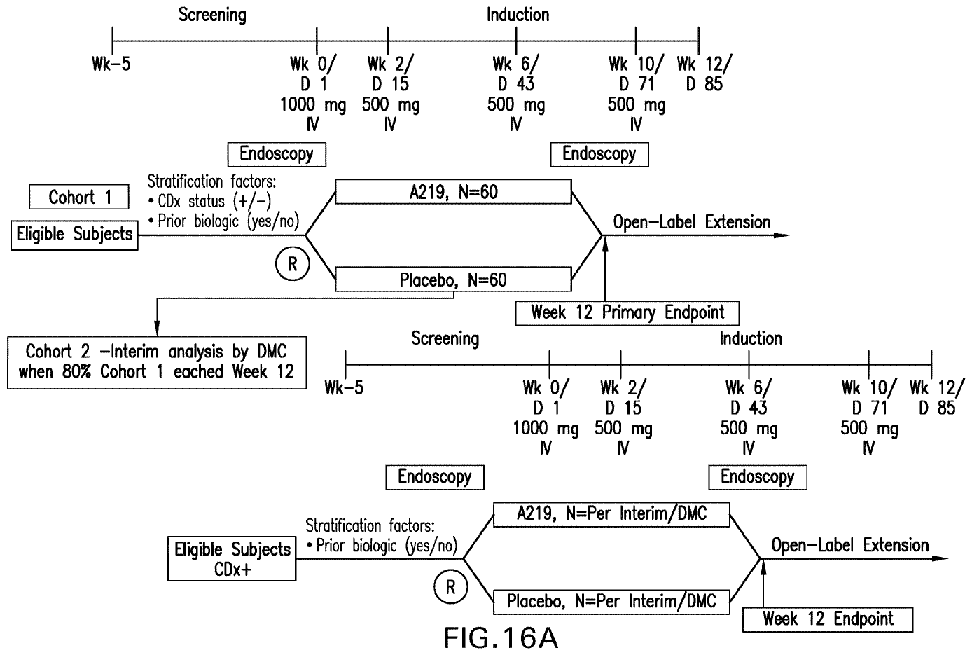

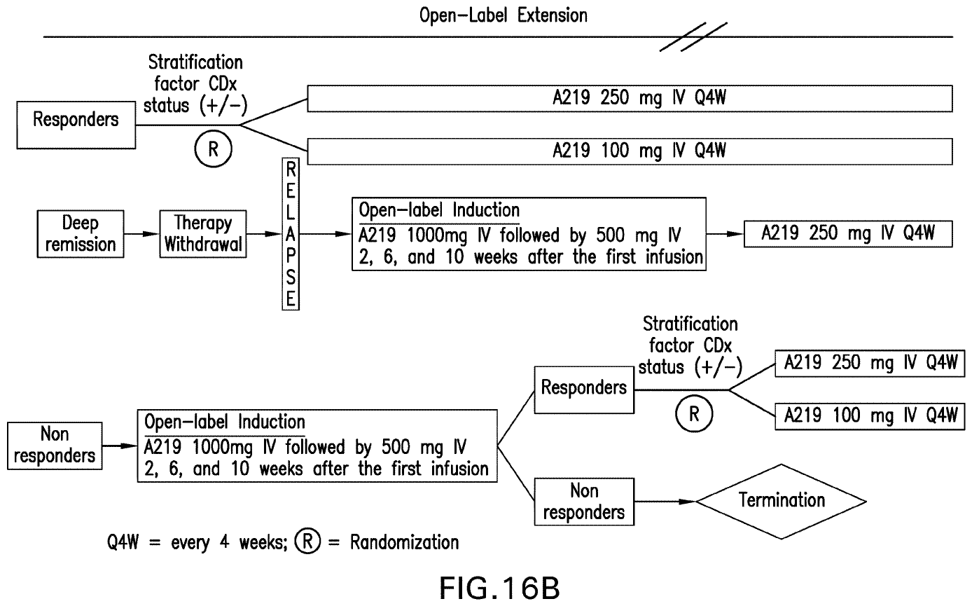

To validate the efficacy of anti-TL1A antibody treatment and the effectiveness of the CDx (e.g. the CDx described in Section 7.12 and 7.13) in selecting responsive patients in ulcerative colitis (UC), a phase 2 clinical trial is conducted. The detailed design of the clinical trial protocol is provided in the protocol synopsis of Table 25 below and shown in FIGS. 16A-16B.

TABLE 25
PROTOCOL SYNOPSIS

| | |
|---|---|
| TITLE: | A Phase 2, Multi-Center, Double-Blind, Placebo-Controlled Study to |
| | Evaluate the CDx (e.g. the CDx described in Section 7.12 and 7.13) |
| | for Treatment with an Anti-TL1A Antibody in Subjects with |
| | Moderately to Severely Active Ulcerative Colitis |
| PROJECT PHASE: | Phase 2 |
| OBJECTIVE: | Primary: |
| | To assess the safety and tolerability of A219 following 12- |
| | weeks ofinduction therapy |
| | To compare the efficacy of A219 vs placebo for induction of |
| | clinicalremission at Week 12 |
| | Secondary: |
| | All objectives below refer to comparison of A219-treated subjects |
| | vs placebo-treated subjects in Cohort 1. For the objectives where |
| | the companion diagnostic (CDx) status is a variable, a comparison |
| | of subjectsin both Cohort 1 and Cohort 2 will be conducted. |
| | To compare the efficacy of A219 vs placebo for induction |
| | ofendoscopic improvement at Week 12 |
| | To compare the efficacy of A219 vs placebo for induction of |
| | clinicalresponse at Week 12 |
| | To compare the efficacy of A219 vs placebo in CDx |
| | positive (CDx+) subjects (Cohort 1 + Cohort 2) for |
| | induction of clinical remission at Week 12 |
| | To compare the efficacy of A219 vs placebo for induction |
| | ofhistologic remission at Week 12 |
| | To compare the efficacy of A219 vs placebo for induction |
| | ofhistologic-endoscopic mucosal improvement at Week 12 |
| | To compare the efficacy of A219 vs placebo in CDx+ |
| | subjects (Cohort 1 + Cohort 2) for induction of endoscopic |
| | improvement at Week 12 |
| | To compare the efficacy of A219 vs placebo in CDx+ |
| | subjects (Cohort 1 + Cohort 2) for induction of clinical |
| | response at Week 12 |
| | To compare the efficacy of A219 vs placebo in CDx+ subjects |
| | (Cohort 1 + Cohort 2) for induction of histologic remission at |
| | Week 12 |
| | To compare the efficacy of A219 vs placebo in CDx+ subjects |
| | (Cohort 1 + Cohort 2) for induction of histologic-endoscopic |
| | mucosalimprovement at Week 12 |
| | To compare the efficacy of A219 in CDx+ (Cohort 1 + Cohort 2) |
| | vsCDx negative (CDx−) subjects for induction of clinical |
| | remission at Week 12 |
| | To compare the efficacy of A219 vs placebo for induction |
| | ofmucosal healing at Week 12 |
| | To compare the efficacy of A219 vs placebo in CDx+ |
| | subjects (Cohort 1 + Cohort 2) for induction of mucosal |
| | healing at Week 12 |
| | To compare the efficacy of A219 vs placebo for change in |
| | Inflammatory Bowel Disease Questionnaire (IBDQ) at |
| | Week 12 |
| | To compare the efficacy of A219 vs placebo in CDx+ |
| | subjects(Cohort 1 + Cohort 2) for change in IBDQ at Week 12 |
| | To compare the efficacy of A219 vs. placebo in subjects who |
| | areCDx+ per alternative algorithm (Cohort 1 + Cohort 2) for |
| | clinical remission at Week 12 |
| | Exploratory: |
| | To assess the pharmacokinetics (PK) of A219 in subjects |
| | withulcerative colitis (UC) over time |
| | To assess the effects of A219 on tissue and serum |
| | pharmacodynamic(PD) markers, including total TL1A |
| | concentrations over time |
| | To assess the effect of A219 on inflammatory biomarkers |
| | includingfecal calprotectin and high sensitivity C-reactive |
| | protein (hsCRP) overtime |
| | To assess the proportion of subjects in 3-component Modified |
| | Mayo Score response, 3-component Modified Mayo Score |
| | remission, endoscopic improvement. Robarts histopathology |
| | index (RHI) histologic remission, Geboes score histologic |
| | remission, and mucosalhealing at Week 50 |
| | To assess the change in Partial Mayo Score over time |
| | To assess the change in Geboes Index and RHI from |
| | Baseline to Week 12 and Week 50 |
| | To assess the exposure-response relationship of A219 on PD |
| | markersover time |
| | To assess the proportion of subjects achieving |
| | corticosteroid-free-remission at Week 50 |
| STUDY DESIGN: | This is a multi-center, double-blind, randomized, placebo-controlled |
| | proof of concept study designed to assess the safety, tolerability, and |
| | efficacy of A219 following 12 weeks of induction therapy in subjects |
| | with UC. Thisstudy will be conducted under the aegis of a Data |
| | Monitoring Committee (DMC) and will commence following the |
| | demonstration of an acceptable safety profile of A219 at a dose of ≥500 |
| | mg in the multiple ascending dose study in normal healthy |
| | volunteers (Example 16). |
| | The study has 4 periods (Screening Period, Induction Period [IP], |
| | Open- Label Extension [OLE] Period, and Follow-Up [FU] Period). |
| | The study will have 2 Cohorts that will enroll subjects in a sequential |
| | fashion utilizingan adaptive design as described below. |
| | Cohort 1: Following the Screening Period, approximately 120 |
| | eligible subjects with moderately to severely active UC will enter |
| | the IP and be randomized in a 1:1 fashion to receive intravenous |
| | (IV) administration of A219 1000 mg on Week 0/Day 1, followed |
| | by 500 mg on Weeks 2, 6, and 10, or placebo at the same |
| | timepoints. Randomization will be stratifiedby CDx status of |
| | positive (CDx+) or negative (CDx−) and prior biologic experience |
| | (yes/no) at Week 0/Day 1. Subjects who discontinue from the study |
| | will have a follow-up period of 12 weeks after last dose. |
| | Cohort 2: When approximately 80% of Cohort 1 subjects (i.e., ~96 |
| | subjects)have reached Week 12 or early terminated from the study, |
| | the DMC will conduct an unblinded analysis of clinical efficacy in |
| | CDx+ subjects and willrecommend whether an expansion to Cohort |
| | 2 is warranted. The planned sample size for CDx+ subjects |
| | (combining Cohort 1 and Cohort 2) will be approximately 40, in the |
| | case where Cohort 2 is initiated. For Cohort 2, eligible subjects (who |
| | must be CDx+) will enter the IP and be randomized in a 1:1 fashion |
| | to receive IV administration of A219 1000 mg on |
| | Week 0/Day 1, followed by 500 mg on Weeks 2, 6, and 10, or |
| | placebo atthe same timepoints. Randomization will be stratified by |
| | prior biologic experience (yes/no) at Baseline. Subjects who |
| | discontinue from the study will have a follow-up period of 12 |
| | weeks after last dose. |
| | Subjects who complete the 12-week IP from either Cohort will have |
| | theoption to enter OLE. During OLE, starting at Week 14 visit: |
| | Subjects who have achieved deep remission (defined as in |
| | clinical remission [endoscopic subscore of 0 or 1, rectal bleeding |
| | subscore of 0,and stool frequency subscore of 0 or 1 and not |
| | greater than Baseline] and RHI <3) will continue in the study |
| | and have therapy withdrawal. |
| | Upon disease relapse (defined as rectal bleeding score of ≥1, |
| | and either hsCRP ≥5 and/or fecal calprotectin ≥250), |
| | subjects can receive another course of induction therapy |
| | (1000 mg IV followedby 500 mg IV 2, 6, and 10 weeks after |
| | the first infusion) followed by maintenance therapy of 250 |
| | mg IV every 4 weeks (Q4W) for atotal of 50 weeks. |
| | Responders (defined as reduction from Baseline ≥2 points and ≥30% |
| | in 3-component Modified Mayo Score, accompanied by a |
| | reduction ≥1in rectal bleeding subscore or absolute rectal |
| | bleeding subscore ≤1) who have not achieved deep remission |
| | will be re-randomized, stratifiedby CDx status of CDx+ or CDx−, |
| | to either 250 mg IV Q4W or 100 mg IV Q4W, starting at Week |
| | 14 until Week 50. |
| | Nonresponders will receive an open-label induction regimen of |
| | 1000 mg of A219 on Week 14, followed by 500 mg on Weeks |
| | 16, 20, and 24. Nonresponders who do not respond at Week 28 |
| | (per investigator discretion) should be discontinued from the |
| | study. |
| | Nonresponders who respond at Week 28 (per investigator |
| | discretion) will be re-randomized to either 250 mg IV Q4W or |
| | 100 mg IV Q4W, starting at Week 28 for a total of 50 weeks. |
| | The study may be amended by the Sponsor to extend the OLE |
| | periodbeyond 50 weeks based on emerging safety data. |
| | The study also includes an optional PK sub-study during the IP for |
| | subjectswho consent to additional PK sampling |
| SAMPLE SIZE: | The study is planned to randomize up to approximately 170 |
| | subjects, approximately 120 in Cohort 1 and up to 50 in Cohort 2. |
| | A sample size of60 per arm in Cohort 1 will enable a statistical |
| | power of >80% for the primary endpoint at 1-sided significance |
| | level of 0.025 using Cochran- Mantel-Haenszel (CMH), assuming |
| | clinical remission rate of 5% for placebo and 24% for A219. |
| | Additionally, the sample size will confer >80% |
| | power to achieve statistical significance for the 1^{st} secondary |
| | endpoint of endoscopic improvement with an overall 1-sided alpha |
| | level of0.025, assuming the endoscopic improvement rates are 15% |
| | and 38% for placebo and A219 groups, respectively. |
| | Additionally, for analyses of the CDx population (combining CDx+ |
| | subjects from Cohort 1 and Cohort 2), a sample size of 40 per arm |
| | will provide a statistical power of ≥80% at a 1-sided alpha level of |
| | 0.025, according to a group sequential design with a non-binding |
| | futility interimanalysis when 18 subjects per arm reach Week 12, |
| | assuming clinical remission rate of 5% for placebo and 31% for A219. |
| SUBJECT TYPE: | Male or female subjects ≥18 years of age with moderately to |
| | severely active UC. |
| FORMULATIONS: | A219 will be supplied in 10 mL vials each containing 500 mg A219 |
| | (60 mg/mL solution) for IV administration after reconstitution. |
| DOSAGE: | Subjects will be stratified by CDx+/CDx− status and prior biologic |
| | experience (yes/no) in a 1:1 ratio to: |
| | A219 1000 mg IV on Week 0/Day 1, followed by 500 mg IV |
| | on Weeks 2, 6, and 10 |
| | Placebo IV on Week 0/Day 1, followed by placebo IV on |
| | Weeks 2, 6, and 10 |
| | During OLE: |
| | Deep remitters will enter the therapy withdrawal period |
| | All subjects who develop disease relapse after therapy |
| | withdrawal will receive another course of induction therapy |
| | (1000 mg IV followed by 500 mg IV 2, 6, and 10 weeks after |
| | the first infusion) followed by maintenance therapy of 250 mg |
| | IV Q4W for a total of 50weeks. |
| | Responders at the end of Week 12 will be stratified by CDx |
| | status ofCDx+ or CDx− and re-randomized to receive one of |
| | the following regimens: |
| | A219 250 mg IV on Week 14 then Q4W until Week 50 |
| | A219 100 mg IV on Week 14 then Q4W until Week 50 |
| | Nonresponders at the end of Week 12 will receive open-label |
| | A219 1000 mg IV on Weck 14, followed by A219 500 mg IV on |
| | Weeks 16, 20, and 24. Subjects who do not respond by Week 26 |
| | should be discontinued from the study. Subjects who respond by |
| | Week 26 will bestratified by CDx status of CDx+ or CDx− and |
| | re-randomized to receiveone of the following regimens: |
| | A219 250 mg IV on Week 28 then Q4W for a total of 50 weeks |
| | A219 100 mg IV on Week 28 then Q4W for a total of 50 weeks |
| ROUTE OF | All study drug will be reconstituted in 250 mL of 0.9% normal |
| ADMINISTRATION: | saline (NS)and will be administered IV over 30 minutes. |
| STUDY ENDPOINTS: | Primary endpoints: |
| | The proportion of subjects reporting adverse events (AEs), |
| | serious adverse events (SAEs), AEs leading to discontinuation, |
| | and markedly abnormal laboratory values. |
| | The proportion of subjects in the 3-component Modified Mayo |
| | Score clinical remission (as defined by endoscopic subscore of 0 |
| | or 1, rectal bleeding subscore of 0, and stool frequency subscore |
| | of 0 or 1 and not greater than Baseline) at Week 12. The 3- |
| | component Modified Mayo Score ranges from 0-9 and includes rectal |
| | bleeding, stool frequency andendoscopic assessment domains. |
| | Secondary endpoints: |
| | The proportion of subjects with endoscopic improvement, as |
| | defined byendoscopy subscore ≤1 with no friability) at Week 12. |
| | The proportion of subjects in 3-component Modified Mayo Score |
| | clinicalresponse at Week 12. The 3-component Modified Mayo |
| | Score clinical response is defined by reduction from Baseline ≥2 |
| | points and ≥30% in3-component Modified Mayo Score, |
| | accompanied by a reduction ≥1 inrectal bleeding subscore or |
| | absolute rectal bleeding subscore ≤1. |
| | The proportion of subjects in the 3-component Modified Mayo |
| | Score clinical remission (as defined by endoscopic subscore of 0 |
| | or 1, rectal bleeding subscore of 0, and stool frequency subscore |
| | of0 or 1 and not greater than Baseline), in CDx+ subjects (Cohort |
| | 1 + Cohort 2) treatedwith A219 compared to CDx+ placebo- |
| | treated subjects at Week 12. The 3-component Modified Mayo |
| | Score ranges from 0-9 and includes rectal bleeding, stool |
| | frequency, and endoscopic assessment domains. |
| | The proportion of subjects with histologic remission (defined |
| | Geboesscore ≤3.1) at Week 12. |
| | The proportion of subjects with histologic-endoscopic mucosal |
| | improvement (defined as Geboes score ≤3.1 and endoscopy |
| | subscore ≤1 with no friability) at Week 12. |
| | The proportion of subjects with endoscopic improvement, as |
| | defined byendoscopy subscore ≤1 with no friability, in CDx+ |
| | subjects (Cohort 1 + Cohort 2) treated with A219 compared |
| | to CDx+ placebo-treatedsubjects at Week 12. |
| | The proportion of subjects in 3-component Modified Mayo Score |
| | clinical response in CDx+ subjects treated with A219 compared |
| | to CDx+ placebo-treated subjects at Week 12. The 3-component |
| | ModifiedMayo Score clinical response is defined by reduction |
| | from Baseline ≥2points and ≥30% in 3-component Modified |
| | Mayo Score, accompanied by a reduction ≥1 in rectal bleeding |
| | subscore or absolute rectal bleeding subscore ≤1. |
| | The proportion of subjects with histologic remission, defined as |
| | Geboesscore ≤3.1, in CDx+ subjects (Cohort 1 + Cohort 2) |
| | treated with A219 compared to CDx+ placebo-treated subjects at |
| | Week 12. |
| | The proportion of subjects with histologic-endoscopic mucosal |
| | improvement (defined as Geboes score ≤3.1 and endoscopy |
| | subscore ≤1 with no friability), in CDx+ subjects (Cohort 1 + |
| | Cohort 2) treatedwith A219 compared to CDx+ placebo-treated |
| | subjects at Week 12. |
| | The proportion of subjects with clinical remission (defined as |
| | endoscopic subscore of 0 or 1, rectal bleeding subscore of 0, |
| | and stoolfrequency subscore of 0 or 1 and not greater than |
| | Baseline) in CDx+ subjects (Cohort 1 + Cohort 2) treated with |
| | A219 compared to in CDx− subjects treated with A219 at Week 12. |
| | The proportion of subjects with mucosal healing (defined as |
| | Geboesscore ≤2B.1 and endoscopy subscore of ≤1) at Week 12. |
| | The proportion of subjects with mucosal healing (defined as |
| | Geboesscore ≤2B.1 and endoscopy subscore of ≤1), in CDx+ |
| | subjects (Cohort 1 + Cohort 2) treated with A219 compared to |
| | CDx+ placebo-treated subjects at Week 12. |
| | The proportion of subjects with IBDQ response, as defined by ≥16- |
| | point increase from Baseline at Week 12. |
| | The proportion of subjects with IBDQ response, as defined by ≥16- |
| | point increase from Baseline, in CDx+ subjects (Cohort 1 + |
| | Cohort 2)treated with A219 compared to CDx+ placebo- |
| | treated subjects at Week 12. |
| | The proportion of subjects in the 3-component Modified Mayo |
| | Score clinical remission (as defined by endoscopic subscore of |
| | 0 or 1, rectalbleeding subscore of 0, and stool frequency |
| | subscore of 0 or 1 and notgreater than Baseline), in CDx+ |
| | subjects per alternative algorithm (Cohort 1 + Cohort 2) treated |
| | with A219 compared to CDx+ |
| | placebo-treated subjects per alternative algorithm at Week 12. |
| | The 3-component Modified Mayo Score ranges from 0-9 and |
| | includes rectal bleeding, stool frequency, and endoscopic |
| | assessment domains. |
| | Exploratory endpoints: |
| | The pharmacokinetics of A219 in subjects with UC after multipledoses |
| | The change from Baseline in serum and fecal inflammatory |
| | biomarkers(PD markers) |
| | The proportion of subjects in 3-component Modified Mayo |
| | Score response, 3-component Modified Mayo Score |
| | remission, endoscopicimprovement, RHI histologic remission, |
| | Geboes score histologic remission, and mucosal healing at |
| | Week 50 |
| | The change in Partial Mayo Score (with or without PGA |
| | component)over time |
| | The change in Geboes Index and RHI from Baseline to Week |
| | 12 and Week 50 |
| | The exposure-response relationship of A219 on PD markers |
| | Within subpopulation of subjects on corticosteroid at study |
| | entry, the proportion of subjects in clinical remission and off of |
| | corticosteroid at Week 50 |
| INCLUSION | Subjects are required to meet the following criteria in order to be |
| CRITERIA: | included inthe study: |
| | 1. Male or female ≥18 years of age. |
| | 2. Subjects must have had a documented diagnosis of UC |
| | (endoscopy + histology) to be eligible for study participation. |
| | For subjects with no documented confirmation of UC |
| | diagnosis or if previous diagnosis isnot deemed conclusive, |
| | UC diagnosis must be confirmed at time of screening colonoscopy. |
| | 3. Moderately to severely active UC as defined by 3-component |
| | ModifiedMayo score (3 components of rectal bleeding, stool |
| | frequency, and endoscopy) of 4 to 9, inclusive, with Modified |
| | Mayo endoscopic subscore ≥2 and rectal bleeding subscore ≥1. |
| | 4. Subjects must satisfy at least one of the following criteria: |
| | a) In the past, had an inadequate response to one or more of |
| | thefollowing treatments: |
| | Oral prednisone ≥40 mg/day (or equivalent) or budesonide ≥9 mg/day |
| | or equivalent or beclomethasone ≥5 mg/day for at least2 weeks |
| | Corticosteroid dependence as defined by failed to successfully |
| | taper to <10 mg/day of prednisone or equivalent (i.e., had a |
| | flare of disease) within 3 months of starting therapy, |
| | or if relapse occurs within 3 months of stopping corticosteroids |
| | Immunosuppressants (azathioprine ≥2 mg/kg/day or |
| | 6-mercaptopurine ≥1.0 mg/kg/day [or documentation |
| | of a therapeutic concentration of 6-thioguanine |
| | nucleotide]) for atleast 8 weeks |
| | An approved anti-TNF agent at an approved labeled dose |
| | for atleast 8 weeks |
| | Vedolizumab at the approved labelled dose for at least 8 weeks |
| | An approved JAK inhibitor (e.g., tofacitinib) at an |
| | approvedlabelled dose for at least 8 weeks |
| | An approved anti-IL-12/23 (e.g., ustekinumab) at an |
| | approvedlabelled dose for at least 8 weeks |
| | An approved sphingosine 1-phosphate receptor |
| | (S1PR) modulator at an approved labelled dose for |
| | least 12 weeks |
| | OR |
| | b) Had been intolerant to one or more of the above-mentioned |
| | treatments (e.g., unable to achieve doses or treatment durations |
| | because of dose limiting side effects [e.g., leukopenia, |
| | psychosis, uncontrolled diabetes, elevated liver enzymes]). |
| | OR |
| | c) Currently receiving one or more of the following treatments: |
| | Oral Prednisone ≥10 mg/day (or equivalent) for at least 3 months |
| | Immunosuppressants [azathioprine ≥2 mg/kg/day or |
| | 6-mercaptopurine ≥1.0 mg/kg/day (or documentation |
| | of a therapeutic concentration of 6-thioguanine |
| | nucleotide)] for atleast 8 weeks. |
| | Notes on subjects who have had prior biologic/biologic-like |
| | therapy(ies) (anti-TNF, JAK inhibitor. S1PR modulator, anti-IL- |
| | 12/23, and/or vedolizumab): |
| | The study will include a maximum of 70% subjects who have |
| | had prior biologic/biologic-like therapy(ies) experience. |
| | Upon reachingthe maximum number of allowed |
| | biologic/biologic-like experienced subjects (70%), subjects |
| | who have had prior biologic/biologic-like experience will no |
| | longer be allowed to enterthe study. |
| | Subject cannot have failed (no response, insufficient response, |
| | lossof response, and/or intolerance) >3 classes or >4 individual |
| | biologic/biologic-like therapies (refer to exclusion criterion #26). |
| | 5. For subjects who are women of childbearing potential |
| | (WOCBP) involved in any sexual intercourse that could lead to |
| | pregnancy, the subject has used two highly effective methods |
| | of contraception for atleast 4 weeks prior to Day 1 and agrees |
| | to continue to use two highly effective methods of contraception |
| | until at least 12 weeks after the lastdose of study drug. |
| | 6. Male subjects must use, with their female partner of |
| | childbearing potential, two highly effective methods of |
| | contraception and refrain from sperm donation from screening |
| | to 12 weeks after the last dose ofstudy drug. |
| | 7. Subject must meet drug stabilization requirements, as applicable: |
| | a) Oral corticosteroid treatment must be equivalent of ≤20 mg |
| | prednisone or ≤9 mg budesonide or beclomethasone ≤5 mg |
| | dailyat a stable dose for at least 2 weeks prior to randomization |
| | b) Oral aminosalicylates should be at a stable dose for at least 2 |
| | weeksprior to randomization |
| | c) Azathioprine and 6-mercaptopurine should be at a stable dose |
| | for atleast 4 weeks prior to randomization |
| | 8. Able to provide written informed consent and understand and |
| | comply with the requirements of the study. |
| | 9. For Cohort 2 only: Subjects must be CDx+. |
| EXCLUSION | Subjects with the following characteristics will be excluded from the |
| CRITERIA: | study: |
| | Sex and Reproductive Status |
| | 1. WOCBP and men with female partners of childbearing potential |
| | who are unwilling or unable to use two highly effective methods |
| | of contraception to avoid pregnancy for the entire study period |
| | and for upto 12 weeks after the last dose of study drug. |
| | 2. Women who are pregnant or breastfeeding. |
| | 3. Women with a positive pregnancy test on enrollment or |
| | prior torandomization. |
| | Target Disease Exceptions |
| | 4. Diagnosis of Crohn's disease or indeterminate colitis. |
| | 5. UC limited to the rectum (<15 cm from anal verge). |
| | 6. Current evidence of fulminant colitis, toxic megacolon, or |
| | bowelperforation. |
| | 7. Current or impending need for colostomy or ileostomy. |
| | 8. Previous total proctocolectomy or partial colectomy. |
| | 9. Surgical bowel resection within 3 months before screening. |
| | 10. Concomitant primary sclerosing cholangitis (PSC) |
| | Medical History and Concurrent Diseases |
| | 11. Past or current evidence of definite low-grade or high-grade |
| | colonicdysplasia that has not been completely removed. |
| | 12. Subjects who are scheduled or anticipate the need for surgery, |
| | asidefrom dermatologic procedures. |
| | 13. Subjects who have a history of clinically significant drug or |
| | alcoholabuse. |
| | 14. Concomitant illness that in the opinion of the Investigator, is |
| | likely torequire systemic glucocorticosteroid therapy during the |
| | study (e.g., moderate to severe asthma). |
| | 15. Current symptoms of severe, progressive, or uncontrolled |
| | renal, hepatic, hematological, pulmonary, cardiac, |
| | neurological, ophthalmologic or cerebral disease. Concomitant |
| | medical conditionsthat in the opinion of the Investigator might |
| | place the subject at unacceptable risk for participation in this study. |
| | 16. Subjects with a history of cancer within the last 5 years (other |
| | than non-melanoma skin cell cancers cured by local resection). |
| | Existing non-melanoma skin cell cancers must be removed |
| | prior to enrollment. Subjects with carcinoma in situ or localized |
| | cervical cancer, treated with definitive surgical intervention, are |
| | allowed. |
| | 17. Subjects at risk for tuberculosis (TB). Specifically, subjects with: |
| | a) A history of active TB |
| | b) Current clinical, radiographic or laboratory evidence of active TB |
| | c) Latent TB which was not successfully treated. Subjects with |
| | a positive TB screening test indicative of latent TB will not |
| | be eligible for the study unless active TB infection has been |
| | ruled out, and an appropriate course of intervention for latent |
| | TB has been initiated at least 2 weeks prior to randomization, |
| | and no evidence of active TB on chest x-ray during Screening. |
| | 18. Subjects with any serious bacterial infection within the last 3 |
| | months, unless treated and resolved with antibiotics, or any |
| | chronic bacterial infection (such as chronic pyelonephritis, |
| | osteomyelitis and bronchiectasis). |
| | 19. Female subjects who have had a breast cancer screening that is |
| | suspicious for malignancy, and in whom the possibility of |
| | malignancy cannot be reasonably excluded following additional |
| | clinical, laboratory or other diagnostic evaluations. |
| | 20. Subjects with any active infections (excluding fungal infections |
| | of nailbeds) including, but not limited to, those that require |
| | intravenous (IV) antimicrobial treatment 4 weeks or oral |
| | antimicrobial treatment 2 weeks prior to randomization. |
| | Subjects with evidence of Human Immunodeficiency Virus |
| | (HIV), Hepatitis B or Hepatitis C infection detected during |
| | screening are also excluded, but subjects with successfully |
| | treated Hepatitis C with no recurrence for ≥1 year are allowed. |
| | Subjects with active documented or suspected COVID-19 |
| | infection within 4 weeks of randomization or asymptomatic |
| | SARS-CoV-2 PCR test within 2 weeks of randomization are excluded. |
| | 21. Subjects with herpes zoster reactivation or cytomegalovirus |
| | (CMV) that resolved less than 2 months prior to signing |
| | informed consent. |
| | 22. Subjects who have received any live vaccines within 3 months |
| | of theanticipated first dose of study medication or who will have |
| | need of alive vaccine at any time during the study. |
| | Physical and Laboratory Test Findings |
| | 23. Positive stool Polymerase Chain Reaction (PCR) or culture for |
| | entericpathogens. |
| | 24. Stool positive for Clostridium difficile (C. difficile) toxin. |
| | Subjects whoare positive can be retested after the completion |
| | of a full course of treatment for C. difficile infection. |
| | 25. Any of the following lab values: |
| | a) Hemoglobin (Hgb) <8.0 g/dL (80 g/L) |
| | b) White blood cell (WBC) <2,500/mm³ (2.5 × 10⁹/L) |
| | c) Neutrophils <1,000/mm³ (1 × 10⁹/L) |
| | d) Platelets <100,000/mm³ (100 × 10⁹/L) |
| | e) Serum creatinine >2 times upper limit of normal (ULN) |
| | f) Serum alanine aminotransferase (ALT) or |
| | aspartateaminotransferase (AST) >2 times ULN |
| | g) Any other laboratory test results that, in the opinion of |
| | the Investigator, might place the subject at |
| | unacceptable risk forparticipation in this study |
| | Prohibited Therapies and/or Medications |
| | 26. Failed (no response, insufficient response, loss of response, |
| | and/or intolerance) >3 classes (anti-TNF, anti-integrin, anti- |
| | IL12/23, JAKinhibitor, S1PR modulator) or >4 individual |
| | biologic/biologic-liketherapies. |
| | 27. Any marketed biologic or biologic-like within 2 weeks for |
| | tofacitinib, 8weeks for anti-TNF agents, 10 weeks for S1PR |
| | modulators, and 12 weeks for vedolizumab and ustekinumab |
| | prior to randomization or if drug level per therapeutic dose |
| | monitoring is greater than lower limit ofdetection. |
| | 28. Any biologic immunomodulators not covered in exclusion |
| | criterion 27, used for UC or other conditions within 8 weeks or 5 |
| | half-lives, whichever is longer, prior to randomization or if drug |
| | level per therapeutic dose monitoring is greater than lower limit |
| | of detection. |
| | 29. Rituximab within 1 year prior to randomization. |
| | 30. Parenteral corticosteroids within 4 weeks or rectal |
| | administration ofcorticosteroids within 2 weeks prior to |
| | randomization. |
| | 31. Rectal administration of 5-ASA within 2 weeks prior to |
| | randomization. |
| | 32. Tacrolimus, methotrexate, cyclosporine, mycophenolate mofetil |
| | (CellCept ®), immunoadsorption columns (such as Prosorba |
| | columns), D Penicillamine, Leflunomide, Thalidomide, fish-oil |
| | preparations, probiotics, fecal transplantation, non-steroidal anti- |
| | inflammatory agents(NSAIDs), aspirin >81 mg/day within 2 |
| | weeks prior to randomization. |
| | 33. Other investigational chemical agent within 30 days or other |
| | investigational biologic agent within 8 weeks or 5 half-lives |
| | (whicheveris longer) of randomization. |
| | 34. Prior exposure to A219. |
| | Other Exclusion Criteria |
| | 35. Prisoners or subjects who are compulsorily detained |
| | (involuntarily incarcerated) for treatment of either a |
| | psychiatric or physical (e.g.,infectious disease) illness must |
| | not be enrolled into this study. |
| | 36. Legal or mental incapacitation, or inability to understand and |
| | comply with the requirements of the study. |
| Statistical | Statistical methods will be detailed in the Statistical Analysis Plan. |
| Methods: | The SAP will provide details about the method of analysis and |
| | specific plannedanalyses, and will be prepared and approved by |
| | Prometheus Biosciences and its designees before study database |
| | lock and unblinding of subject treatment assignments. |
| | The analysis populations are defined as follows: |
| | Full analysis set (FAS) from Cohort 1: all subjects |
| | randomized andtreated in Cohort 1 |
| | FAS for CDx+: all CDx+ subjects who are randomized and |
| | treated inboth Cohort 1 and Cohort 2 |
| | Safety analysis set: all subjects treatedThe following analyses |
| | will be performed: |
| | Efficacy: |
| | The efficacy assessment will test for the difference between A219 |
| | andplacebo groups in FAS. |
| | The primary endpoint will be analyzed and compared between A219 |
| | andplacebo treatment groups in FAS from Cohort 1. The primary |
| | endpoint, the proportion of subjects achieving clinical remission, will |
| | be tested between the 2 treatment groups at 1-sided significance level |
| | of 0.025 using CMH with stratification factors at randomization. If |
| | significant, the 1^{st} secondary endpoint of proportion of subjects |
| | achieving endoscopic improvement will be tested between the 2 |
| | treatment groups at 1-sided significance level of |
| | 0.025. If significant, the 2^{nd}, 3^{rd}, 4^{th}, and 5^{th} secondary endpoints |
| | will be tested sequentially, each at 1-sided significance level of |
| | 0.025. Testing forstatistical significance will stop when the first |
| | endpoint is not statistically significant at level of 0.025 and all |
| | remaining p values will be nominal. |
| | Treatment difference for primary and secondary endpoints for Cohort |
| | 1 willbe estimated along with 95% CI for all subjects in FAS. The |
| | secondary endpoints in CDx+ subjects will be summarized and |
| | compared between A219 and placebo groups in FAS for CDx+, |
| | while the treatment difference will be estimated with 95% CI. |
| | Additional efficacy analysis will be detailed in SAP. |
| | Interim Efficacy Analysis: |
| | An interim analysis will be carried out when approximately 80% of |
| | subjects (approximately 96 subjects) in Cohort 1 have reached |
| | Week 12 orearly terminated from the study. The DMC will review |
| | the unblinded efficacy and safety data and recommend on the |
| | expansion to Cohort 2. |
| | Decision rules to initiate Cohort 2 are determined according to the |
| | futility bounds of group sequential design of a sample size of 40 per |
| | arm with oneinterim analysis at the information fraction of 45%. |
| | Because the exact bounds will be calculated using the actual number |
| | of subjects with CDx+ included in the interim analysis, the final |
| | decision rules, along with sensitivity analysis, will be specified in |
| | the DMC SAP, prior to the interimanalysis. |
| | Adverse Events: |
| | AEs will be coded using the most current version of Medical |
| | Dictionary for Regulatory Activities (MedDRA ®) |
| | A by-subject AE data listing, including verbatim term, preferred |
| | term (PT), system organ class (SOC), treatment, severity, seriousness |
| | criteria, relationship to drug, and action taken, will be provided. |
| | The number of subjects experiencing treatment-emergent adverse |
| | events(TEAEs) and number of TEAEs will be summarized by |
| | treatment using frequency counts for Safety analysis set. |
| | Medical History, chest x-ray, electrocardiogram (ECG), and |
| | physicalexamination will be listed by subject. |
| | Changes in ECGs and physical examinations will be described in the |
| | text ofthe final report. |
| | Concomitant Medications: |
| | Concomitant medications will be coded using the most current |
| | WorldHealth Organization (WHO) drug dictionary and listed |
| | by treatment. |
| | Pharmacokinetics: |
| | Summary statistics of A219 concentrations and anti-drug |
| | antibody (ADA) by visit and by CDx+ and CDx− |

### 7.10 Example 10: A Phase 2a, Multi-Center, Open-Label Clinical Trial of A219 in Subjects with Moderately to Severely Active Crohn's Disease

To evaluate the effectiveness of the CDx markers and models for selecting patients responsive to treatment of an anti-TL1A antibody, a phase 2 clinical trial with a representative anti-TL1A antibody, A219 as described herein. The detailed design of the clinical trial protocol is shown in the protocol synopsis in Table 26 below.

TABLE 26
Protocol Synopsis of Phase 2A Trial for CDx and A219 in CD Patients

| | |
|---|---|
| TITLE: | A Phase 2a, Multi-Center, Open-Label Study to Evaluate the Safety, |
| | Efficacy, and Pharmacokinetics of A219 in Subjects with Moderately to |
| | Severely Active Crohn's Disease |
| PROJECT PHASE: | Phase 2a |
| OBJECTIVE: | Primary: |
| | To evaluate the safety and tolerability of A219 following 12-weeksof |
| | induction therapy |
| | To assess the proportion of subjects with endoscopic improvement |
| | (decrease in simple endoscopy score for Crohn's disease [SES-CD] ≥50% |
| | from Baseline) at Week 12 |
| | Secondary: |
| | To assess the proportion of subjects with clinical remission |
| | (Crohn's disease activity index [CDAI] <150) at Week 12 |
| | To assess the proportion of subjects with endoscopy and clinical |
| | improvement (decrease in SES-CD ≥50% AND reduction in CDAI ≥100 |
| | points from Baseline) at Week 12 |
| | To assess the proportion of subjects with biomarker and clinical |
| | improvement (decrease in high sensitivity C-reactive protein [hsCRP] or |
| | fecal calprotectin ≥50% from Baseline, among subjects with at leastone |
| | elevated biomarker at Baseline, AND reduction in CDAI ≥100 points from |
| | Baseline) at Week 12 |
| | ≥100 points from Baseline) at Week 12 |
| | To assess the proportion of subjects with normalization of C-reactive |
| | protein (hsCRP < upper limit of normal [ULN]), among subjects |
| | with elevated concentrations at Baseline, at Week 12 |
| | To assess the proportion of subjects with normalization of fecal |
| | calprotectin (fecal calprotectin < ULN), among subjects with elevated |
| | concentrations at Baseline, at Week 12 |
| | To assess the proportion of subjects with clinical |
| | improvement (reduction in CDAI ≥100 points from Baseline) at Week 12 |
| | To assess the proportion of subjects with two component patient-reported |
| | outcome (PRO-2) remission (average daily abdominal pain score ≤1 |
| | point and average daily stool frequency ≤3 points with abdominal pain |
| | and stool frequency no worse than Baseline at Week 12) |
| | To assess the change in SES-CD score from Baseline to Week 12 |
| | To assess the pharmacokinetics (PK) of A219 |
| | To assess the immunogenicity of A219 |
| | Exploratory: |
| | To assess the change in CDAI and component scores over time |
| | To assess the effects of A219 on tissue and serum pharmacodynamic (PD) |
| | markers, including tumor necrosis factor-like cytokine 1A (TL1A) |
| | concentrations, endoscopic healing index (EHI), fecal calprotectin, and |
| | hsCRP in all subjects over time |
| | To assess the change in SES-CD at Week 50 from Baseline |
| | To characterize the change in Perianal Disease Activity Index |
| | (PDAI) score from Baseline to Week 12, Week 28, and Week 50 |
| | To characterize the effect of A219 for improvement and remissionof |
| | enterocutaneous and/or perianal fistula during the Induction Period(IP) and |
| | Open-Label Extension (OLE) |
| | To assess all secondary endpoints at Week 50 |
| | To assess the change in global histological activity score (GHAS) |
| | and Robarts histopathology index (RHI) from Baseline to Week 12 and Week 50 |
| | To assess the proportion of subjects with histologic response |
| | and histologic remission at Week 12 and Week 50 |
| | To assess change in PRO-2 over time |
| | To assess change in extraintestinal manifestations over time |
| STUDY DESIGN: | This is a multi-center, open-label, proof of concept study designed to |
| | assess the safety, tolerability, and preliminary efficacy of A219 following |
| | 12 weeks of induction therapy in subjects with Crohn's disease (CD). |
| | This study will be conducted under the aegis of a Data Monitoring Committee |
| | (DMC) and will commence following the demonstration of an acceptable |
| | safety profile of A219 at a dose of ≥500 mg in the multiple ascending dose |
| | study in normal healthy volunteers (Study PR200-101). |
| | The study has 4 periods (Screening, IP, OLE and Follow-Up [FU] Period). |
| | Following the Screening Period, approximately 50 eligible subjects with |
| | moderately to severely active CD will enter the IP to receive A219 1000 mg |
| | on Week 0/Day 1, followed by 500 mg on Weeks 2, 6, and 10 via |
| | intravenous (IV) administration. Subjects who discontinue from the study |
| | will have a follow-up period of 12 weeks after the last dose. |
| | Response at Week 12 will be defined as reduction from Baseline in |
| | CDAI of ≥100 points. Non-responders at Week 12 should discontinue |
| | from the study. |
| | Subjects who complete the 12-week IP and have responded will have |
| | theoption to enter OLE. Subjects will continue in OLE until they progress, |
| | withdraw from the study, study termination, or Week 50. During the OLE, |
| | starting at Week 14 visit: |
| | Subjects who achieved deep remission (defined as clinical remission |
| | with CDAI <150, endoscopic remission with SES-CD ≤4 with none of |
| | the segments with score of more than 1, and RHI <3) will continuein the |
| | study and have therapy withdrawal |
| | Upon disease relapse (defined as CDAI ≥220 and either hsCRP ≥5 |
| | mg/L and/or fecal calprotectin ≥250 μg/g), subjects can receive |
| | another course of induction therapy (1000 mg IV followedby 500 mg IV 2, |
| | 6, and 10 weeks after the first infusion) followed by maintenance therapy |
| | of 250 mg IV every 4 weeks (Q4W) for atotal of 50 weeks |
| | Other subjects will be re-randomized to either 250 mg IV Q4W or |
| | 100 mg IV Q4W until Week 50 |
| | The study may be amended by the Sponsor to extend the OLE |
| | period beyond 50 weeks based on emerging safety data. |
| SAMPLE SIZE: | The study is planned to enroll approximately 50 subjects. The sample size |
| | will enable a statistical power of 80%, at 1-sided significance level of 0.025, |
| | to test against the null hypothesis of endoscopic improvement rate of12%, |
| | assuming the endoscopic improvement rate for A219 is 27%. |
| SUBJECT TYPE: | Male or female subjects ≥18 years of age with moderately to |
| | severelyactive CD. |
| FORMULATIONS: | A219 will be supplied in 10 mL vials each containing 500 mg A219(60 |
| | mg/mL solution) for IV administration after reconstitution. |
| DOSAGE: | Induction Period: all subjects will receive A219 1000 mg on Week |
| | 0/Day 1, followed by 500 mg IV on Weeks 2, 6, and 10. |
| | Non-responders at Week 12 should be discontinued from the study. |
| | Responders at the end of Week 12 have the option to enter the OLE, |
| | where subjects with deep remission will undergo therapy withdrawal and |
| | subjects without deep remission will be randomized to receive one of the |
| | following regimens until disease progression, withdraw from the study, |
| | study termination, or Week 50: |
| | A219 250 mg IV on Week 14 then Q4W |
| | A219 100 mg IV on Week 14 then Q4W |
| | Subjects who do not respond by the end of Week 12 should be |
| | discontinued from the study. |
| | All subjects who develop disease relapse (defined as CDAI ≥220 and either |
| | hsCRP ≥5 mg/L and/or fecal calprotectin ≥250 μg/g), after therapy |
| | withdrawal will receive another course of induction therapy (1000 mg IV |
| | followed by 500 mg IV 2, 6, and 10 weeks after the first infusion) |
| | followed by maintenance therapy of 250 mg IV Q4W for a total of 50 weeks. |
| ROUTE OF | The study drug will be reconstituted in 250 mL of 0.9% normal saline |
| ADMINISTRATION: | (NS) and will be administered IV over 30 minutes. |
| STUDY ENDPOINTS: | Primary endpoints: |
| | Safety and tolerability: the proportion of subjects reporting |
| | adverse events (AEs), serious adverse events (SAEs), AEs leading to |
| | discontinuation, and markedly abnormal laboratory values |
| | The proportion of subjects with endoscopic improvement, as defined |
| | by decrease in SES-CD ≥50% from Baseline at Week 12 |
| | Secondary endpoints: |
| | The proportion of subjects in clinical remission (CDAI <150) at |
| | Week 12 |
| | The proportion of subjects with endoscopic and clinical improvement, |
| | as defined by decrease in SES-CD ≥50% AND reduction in CDAI ≥100 |
| | points from Baseline at Week 12 |
| | The proportion of subjects with both biomarker and clinical |
| | improvement (decrease in hsCRP OR fecal calprotectin ≥50% from |
| | Baseline, among subjects with at least one elevated biomarker at |
| | Baseline, AND reduction in CDAI ≥100 points from Baseline) at Week 12 |
| | The proportion of subjects with normalization of hsCRP (as defined |
| | by hsCRP < ULN), among subjects with elevated concentrations at Baseline, |
| | at Week 12 |
| | The proportion of subjects with normalization of fecal calprotectin |
| | (as defined by fecal calprotectin < ULN), among subjects with elevated |
| | concentrations at Baseline, at Week 12 |
| | The proportion of subjects in clinical response, as defined by |
| | reduction in CDAI ≥100 points from Baseline at Week 12 |
| | The proportion of subjects with PRO-2 remission (defined as average |
| | daily abdominal pain score ≤1 point and average daily stool |
| | frequency ≤3 points with abdominal pain and stool frequency no worse |
| | than Baseline) at Week 12 |
| | Change in SES-CD score at Week 12 from Baseline |
| | Descriptive summaries of PK and immunogenicity of A219 |
| | Proportion of subjects developing anti-drug antibody (ADA) |
| | and neutralizing antibody (Nab) |
| | Exploratory endpoints: |
| | Change in CDAI and components from Baseline over time |
| | Change in PD markers including TL1A concentrations, EHI, |
| | fecal calprotectin, and hsCRP over time |
| | Change in SES-CD from Baseline at Week 50 |
| | Change in PDAI from Baseline over time |
| | Proportion of subjects with improvement or remission of |
| | enterocutaneous and/or perianal fistula at Week 12 and Week 50 |
| | Change in GHAS and RHI from Baseline to Week 12 and Week 50 |
| | The proportion of subjects with GHAS histologic score ≤4 at Week |
| | 12 and Week 50 |
| | The proportion of subjects with Robarts histologic score <5 at Week |
| | 12 and Week 50 |
| | The proportion of subjects with GHAS histologic remission, defined |
| | as no neutrophils in the epithelium or subscore of 0, at Week 12 and Week 50 |
| | The proportion of subjects with Robarts histologic remission (<3) at |
| | Week 12 and Week 50 |
| | Time to relapse among subjects with deep remission at Week 12 |
| | Proportion of subjects who relapse, among subjects who achieved |
| | deepremission at Week 12 |
| | Change in PRO-2 over time |
| | The proportion of subjects with extraintestinal manifestation |
| | throughWeek 50 |
| INCLUSION | Subjects are required to meet the following criteria in order to be |
| CRITERIA: | included in the study: |
| | 1. Male or female ≥18 years of age. |
| | 2. Subjects must have had a documented diagnosis of CD (endoscopy + |
| | histology) to be eligible for study participation. For subjects with no |
| | documented confirmation of CD diagnosis or if previous diagnosis isnot |
| | deemed conclusive, CD diagnosis must be confirmed at time of screening |
| | colonoscopy. |
| | 3. Moderately to severely active CD as defined by CDAI of ≥220 and ≤450. |
| | 4. SES-CD score (per central reading) ≥6 if ileocolonic or |
| | colonicdisease; or ≥4 if isolated ileal disease only. |
| | 5. Subjects must satisfy at least one of the following criteria: |
| | a) In the past, had an inadequate response to one or more of |
| | the following treatments: |
| | Oral prednisone ≥40 mg/day (or equivalent) or budesonide ≥9 |
| | mg/day or equivalent or beclomethasone ≥5 mg/day for atleast 2 weeks |
| | Corticosteroid dependence as defined by failed to successfully taper |
| | to <10 mg/day of prednisone or equivalent (i.e., had a flare of |
| | disease) within 3 months of starting therapy, or if relapse occurs within 3 |
| | months of stopping corticosteroids |
| | Immunosuppressants (azathioprine ≥2 mg/kg/day or |
| | 6-mercaptopurine ≥1.0 mg/kg/day, [or documentation of a therapeutic |
| | concentration of 6-thioguanine nucleotide] or methotrexate ≥15 |
| | mg/week) for at least 8 weeks |
| | An approved anti-TNF agent at an approved labeled dose for |
| | at least 8 weeks |
| | An approved anti-integrin (e.g., vedolizumab) at an |
| | approved labeled dose for at least 8 weeks |
| | An approved anti-IL-12/23 (e.g., ustekinumab) at an |
| | approved labeled dose for at least 8 weeks |
| | OR |
| | b) Had been intolerant to one or more_of the above mentioned |
| | treatments (e.g., unable to achieve doses or treatment durations because of |
| | dose-limiting side effects [e.g., leukopenia, psychosis, uncontrolled |
| | diabetes, elevated liver enzymes]) |
| | OR |
| | c) Currently receiving one or more of the following treatments: |
| | Oral Prednisone ≥20 mg/day (or equivalent) or budesonide ≥3 |
| | mg/day for at least 4 weeks |
| | Immunosuppressants [azathioprine ≥2 mg/kg/day or |
| | 6-mercaptopurine ≥1.0 mg/kg/day, (or documentation of a therapeutic |
| | concentration of 6-thioguanine nucleotide)] for atleast 8 weeks |
| | Notes on subjects who have had prior approved biologic therapy(ies) |
| | (e.g., anti-TNF, anti-integrin, and/or anti-IL-12/23): |
| | The study will include a maximum of 70% subjects who have had |
| | prior approved biologic therapy(ies) experience. Upon reaching the |
| | maximum number of allowed biologic experienced subjects (70%), subjects |
| | who have had prior biologic experience will no longer be allowed to enter |
| | the study. |
| | Subjects cannot have had failed (no response, insufficient response, |
| | loss of response, and/or intolerance) >4 approved biologic therapies, |
| | whether of same or different mechanism of action |
| | Subjects previously on clinical trials only (i.e., did not receive |
| | commercial available therapy post-approval) are not considered tohave |
| | received the approved therapy for purpose of this inclusion criteria |
| | 6. For subjects who are women of childbearing potential (WOCBP) |
| | involved in any sexual intercourse that could lead to pregnancy, the |
| | subject has used two highly effective methods of contraception for at least |
| | 4 weeks prior to Day 1 and agrees to continue to use two highly effective |
| | methods of contraception until at least 12 weeks after the last dose of study |
| | drug. |
| | 7. Male subjects must use, with their female partner of childbearing |
| | potential, two highly effective methods of contraception and refrain from |
| | sperm donation from screening to 12 weeks after the last dose of study |
| | drug. |
| | 8. Subjects must meet drug stabilization requirements, as applicable: |
| | a) Oral corticosteroid treatment must be equivalent of ≤20 mg |
| | prednisone or ≤9 mg budesonide or beclomethasone ≤5 mg dailyat a |
| | stable dose for at least 2 weeks prior to Day 1 |
| | b) Oral aminosalicylates should be at a stable dose for at least 2 |
| | weeks prior to Day 1 |
| | c) Azathioprine, 6-mercaptopurine, and methotrexate should be at |
| | astable dose for at least 4 weeks prior to Day 1 |
| | 9. Able to provide written informed consent and understand and |
| | comply with the requirements of the study. |
| EXCLUSION | Subjects with the following characteristics will be excluded from the study: |
| CRITERIA: | Sex and Reproductive Status |
| | 1. WOCBP and men with female partners of childbearing potential who |
| | are unwilling or unable to use two highly effective methods of |
| | contraception to avoid pregnancy for the entire study period and for up to |
| | 12 weeks after the last dose of study drug. |
| | 2. Women who are pregnant or breastfeeding. |
| | 3. Women with a positive pregnancy test on enrollment or prior to Day 1. |
| | Target Disease Exceptions |
| | 4. Diagnosis of ulcerative colitis or indeterminate colitis. |
| | 5. CD isolated to the stomach, duodenum, jejunum, or perianal region, |
| | without colonic and/or ileal involvement. |
| | 6. Suspected or diagnosed intra-abdominal or perianal abscess |
| | at Screening. |
| | 7. Known symptomatic stricture or stenosis not passable in |
| | endoscopy (including pediatric colonoscope). |
| | 8. Current stoma or need for colostomy or ileostomy. |
| | 9. Previous small bowel resection with combined resected length of >100 |
| | cm or previous colonic resection of >2 segments. |
| | 10. Currently receiving total parenteral nutrition. |
| | 11. Surgical bowel resection within 3 months before screening. |
| | 12. Concomitant primary sclerosing cholangitis (PSC). |
| | Medical History and Concurrent Diseases |
| | 13. Past or current evidence of definite low-grade or high-grade |
| | colonic dysplasia that has not been completely removed. |
| | 14. Subjects who are scheduled or anticipate the need for surgery, |
| | aside from dermatologic procedures. |
| | 15. Subjects who have a history of clinically significant drug or |
| | alcohol abuse. |
| | 16. Concomitant illness that in the opinion of the Investigator, is |
| | likely to require systemic glucocorticosteroid therapy during the |
| | study (e.g., moderate to severe asthma). |
| | 17. Current symptoms of severe, progressive, or uncontrolled renal, |
| | hepatic, hematological, pulmonary, cardiac, neurological, |
| | ophthalmologic, or cerebral disease. Concomitant medical conditions that |
| | in the opinion of the Investigator might place the subject at unacceptable |
| | risk for participation in this study. |
| | 18. Subjects with a history of cancer within the last 5 years (other than |
| | non-melanoma skin cell cancers cured by local resection). Existing non- |
| | melanoma skin cell cancers must be removed prior to enrollment. Subjects |
| | with carcinoma in situ or localized cervical cancer, treated with definitive |
| | surgical intervention, are allowed. |
| | 19. Subjects at risk for tuberculosis (TB). Specifically, subjects with: |
| | a) A history of active TB |
| | b) Current clinical, radiographic, or laboratory evidence of active TB |
| | c) Latent TB which was not successfully treated. Subjects with a |
| | positive TB screening test indicative of latent TB will not be eligible for the |
| | study unless active TB infection has been ruled out, and an appropriate |
| | course of intervention for latent TB has been initiated at least 2 weeks prior |
| | to Day 1, and no evidence of active TB on chest x-ray during screening. |
| | 20. Subjects with any serious bacterial infection within the last 3 months, |
| | unless treated and resolved with antibiotics, or any chronic bacterial |
| | infection (such as chronic pyelonephritis, osteomyelitis, and |
| | bronchiectasis). |
| | 21. Female subjects who have had a breast cancer screening that is |
| | suspicious for malignancy, and in whom the possibility of malignancy |
| | cannot be reasonably excluded following additional clinical, laboratory, or |
| | other diagnostic evaluations. |
| | 22. Subjects with any active infections (excluding fungal infections of |
| | nailbeds) including, but not limited to, those that require IV antimicrobial |
| | treatment 4 weeks or oral antimicrobial treatment 2 weeks prior to |
| | randomization. Subjects with evidence of Human Immunodeficiency |
| | Virus (HIV), Hepatitis B, or Hepatitis C infection detected during screening |
| | are also excluded, but subjects with successfully treated Hepatitis C with no |
| | recurrence for ≥1 year are allowed. Subjects with active documented or |
| | suspected COVID-19 infection within 4 weeks of randomization or |
| | asymptomatic SARS-CoV-2 PCR test within 2 weeks of randomization are |
| | excluded. |
| | 23. Subjects with herpes zoster reactivation or cytomegalovirus |
| | (CMV) that resolved less than 2 months prior to signing informed consent. |
| | 24. Subjects who have received any live vaccines within 3 months of |
| | theanticipated first dose of study medication or who will have need of |
| | alive vaccine at any time during the study. |
| | Physical and Laboratory Test Findings |
| | 25. Positive stool Polymerase Chain Reaction (PCR) or culture for |
| | enteric pathogens. |
| | 26. Stool positive for Clostridium difficile (C. difficile) toxin. Subjects |
| | whoare positive can be retested after the completion of a full course of treatment |
| | for C. difficile infection. |
| | 27. Any of the following lab values: |
| | a) Hemoglobin (Hgb) <8.0 g/dL (80 g/L) |
| | b) White blood cell (WBC) <2,500/mm³ (2.5 × 10⁹/L) |
| | c) Neutrophils <1,000/mm³ (1 × 10⁹/L) |
| | d) Platelets <100,000/mm³ (100 × 10⁹/L) |
| | d) Serum creatinine >2 times ULN |
| | e) Serum alanine aminotransferase (ALT) or |
| | aspartateaminotransferase (AST) >2 times ULN |
| | f) Any other laboratory test results that, in the opinion of the |
| | Investigator, might place the subject at unacceptable risk for |
| | participation in this study. |
| | Prohibited Therapies and/or Medications |
| | 28. Failed (no response, insufficient response, loss of response, and/or |
| | intolerance) >4 approved biologic therapies (anti-TNF, anti-integrin, |
| | anti-IL12/23), whether of same or different mechanism of action. |
| | 29. Any marketed biologic within 8 weeks for anti-TNF agents and 12 |
| | weeks for anti-integrin agents (e.g., vedolizumab) and ustekinumabprior |
| | to Day 1 or if drug level per therapeutic dose monitoring is greater than |
| | lower limit of detection. |
| | 30. Any biologic immunomodulators used for CD or other conditions |
| | within 8 weeks or 5 half-lives, whichever is longer, prior to Day 1 or if drug |
| | level per therapeutic dose monitoring is greater than lower limit ofdetection. |
| | 31. Rituximab within 1 year prior to Day 1. |
| | 32. Parenteral corticosteroids within 4 weeks or rectal administration |
| | ofcorticosteroids within 2 weeks prior to Day 1. |
| | 33. Rectal administration of 5-ASA within 2 weeks prior to Day 1. |
| | 34. Tacrolimus, cyclosporine, mycophenolate mofetil (CellCept ®), |
| | immunoadsorption columns (such as Prosorba columns), D Penicillamine, |
| | Leflunomide, Thalidomide, chronic use of non-steroidal anti-inflammatory |
| | agents (NSAIDs), and aspirin >81 mg/day within 2 weeks prior to Day 1. |
| | 35. Other investigational chemical agent within 30 days or other |
| | investigational biologic agent within 8 weeks or 5 half-lives (whichever is |
| | longer) of entry into the IP. |
| | 36. Prior exposure to A219. |
| | Other Exclusion Criteria |
| | 37. Prisoners or subjects who are compulsorily detained |
| | (involuntarily incarcerated) for treatment of either a psychiatric or physical |
| | (e.g., infectious disease) illness. |
| | 38. Legal or mental incapacitation, or inability to understand and |
| | comply with the requirements of the study. |
| Statistical Methods: | Statistical methods will be detailed in the Statistical Analysis Plan (SAP). |
| | The SAP will provide details about methods of analysis and the specific |
| | planned analyses, and will be prepared and approved by Prometheus |
| | Biosciences and its designees before study database lock. |
| | The analysis populations are defined as follows: |
| | Full analysis set (FAS): all subjects treated with Baseline SES-CD score |
| | Safety analysis set: all subjects treated |
| | The following analyses will be performed: |
| | Efficacy: |
| | The primary efficacy endpoint, endoscopic improvement at Week 12, will |
| | be used to assess the efficacy of A219. The proportion of subjects in FAS |
| | with endoscopic improvement will be tested against the null hypothesis of |
| | endoscopic improvement rate of 12%, at a 1-sided significance level of |
| | 0.025. If significant, the 1^{st} secondary endpoint of proportion of subjects |
| | achieving clinical remission will be tested against thenull hypothesis of |
| | clinical remission rate of 16%, at a 1-sided significance level of 0.025. |
| | The point estimates for the primary and secondary endpoints will be |
| | calculated along with 90% confidence interval for FAS and by |
| | companion diagnostic (CDx) status (CDx+ or CDx−). |
| | Adverse Events: |
| | AEs will be coded using the most current version of Medical Dictionary |
| | for Regulatory Activities (MedDRA ®). |
| | A by-subject AE data listing, including verbatim term, preferred term (PT), |
| | system organ class (SOC), treatment, severity, seriousness criteria, |
| | relationship to drug, and action taken, will be provided. |
| | The number of subjects experiencing treatment-emergent adverse |
| | events (TEAEs) and number of TEAEs will be summarized by treatment |
| | using frequency counts in safety analysis set. |
| | Medical History, chest x-ray, electrocardiogram (ECG), and |
| | physical examination will be listed by subject. |
| | Changes in ECGs and physical examinations will be described in the text |
| | of the final report. |
| | Concomitant Medications: |
| | Concomitant medications will be coded using the most current World |
| | Health Organization (WHO) drug dictionary and listed by treatment. |
| | Pharmacokinetics: |
| | Summary statistics of A219 concentrations and ADA by visit. |

### 7.11 Example 11: Analyses of the TL1A Isoform Expression

The disclosure provides TNFSF15 gene has at least 2 transcript isoforms: a long isoform (also known and referred to as the canonical isoform) that encodes the TL1A protein sequence (e.g. as set forth in SEQ ID NO: 2050), and a short isoform that encodes a protein that misses amino acids 1-77 from the N-terminus of the canonical TL1A and has difference sequences at amino acids 78-100 of the canonical TL1A (short isoform as set forth in SEQ ID NO: 2052, in which amino acids 1-27 are imputed signal peptide sequence). One exemplary cDNA sequence derived from mRNA sequence for the canonical TL1A isoform is set forth in SEQ ID NO: 2057. One exemplary cDNA sequence derived from mRNA sequence for short TL1A isoform is set forth in SEQ ID NO:2058 (in the translation of which the third amino acid in signal peptide is an His instead of an Arg). The third isoform set forth in SEQ ID NO: 2051 was not observed by the inventors in gastrointestinal tissues in IBD patients.

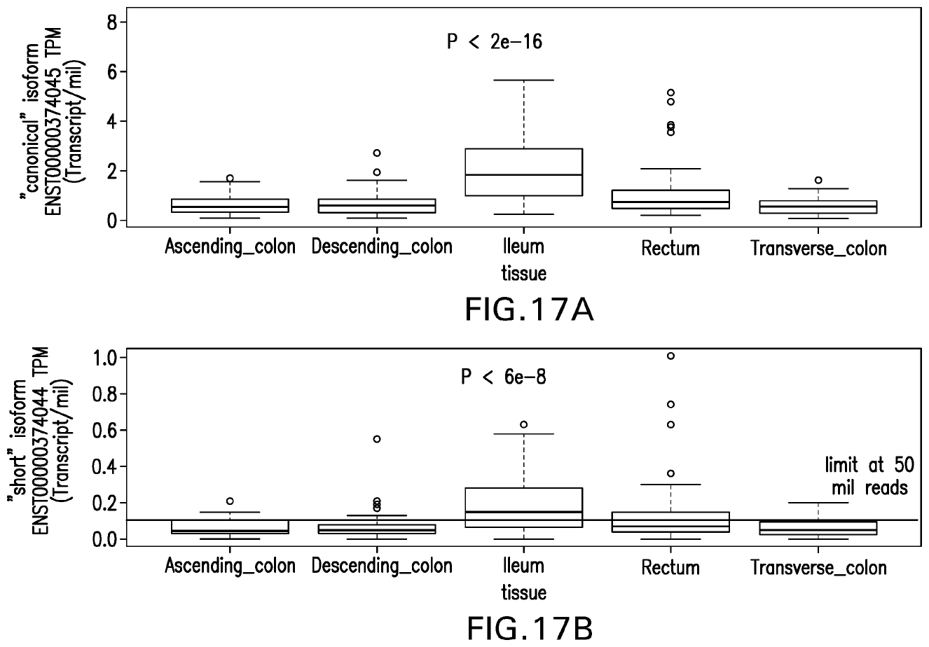

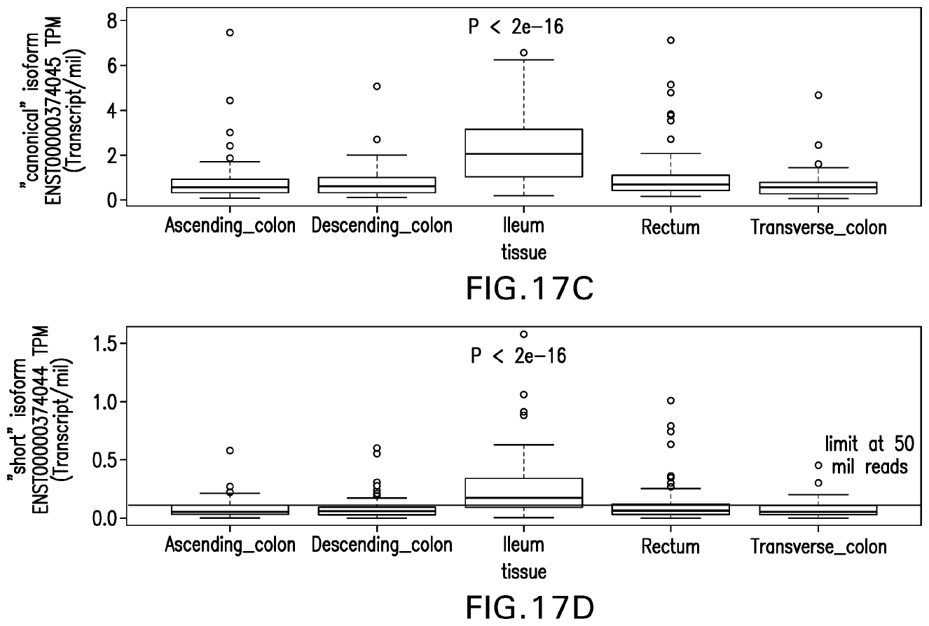

Recognizing TL1A is a cytokine involved in the pathogenesis of IBD, including UC and CD, the inventors first analyzed which gastrointestinal tissue or tissues expressed the TL1A isoform mRNA using RNA sequencing (RNAseq). Briefly, RNA was prepared from the tissue biopsy from patients recruited in clinical trials described above and subjected to next generation sequencing. As shown in FIG. 17A, the long/canonical TL1A isoform is expressed in colon tissues (ascending colon, descending colon, and transverse colon), ileum tissues, and rectum tissues, which were biopsied from CD patients at the screening stage (before TL1A inhibitor treatment), with the highest mRNA level detected in ileum, based on the number of transcripts in the RNAseq. Similarly, as shown in FIG. 17B, the short TL1A isoform is expressed in colon tissues (ascending colon, descending colon, and transverse colon), ileum tissues, and rectum tissues, which were biopsied from CD patients at the screening stage (before TL1A inhibitor treatment), with the highest mRNA level detected in ileum, based on the number of transcripts in the RNAseq. Additionally, inventors examined the mRNA expression level in biopsy tissues both before and after the treatment, similar tissue expression patterns for TL1A long/canonical isoform (FIG. 17C) and TL1A short isoform (FIG. 17D) were observed.

The disclosure provides that the level of the transcripts of the TL1A short isoform is close to the approximate detection limit if the RNAseq is performed at 50 million reads, which is above typical RNA-seq depth less than 50 million (often less than 30 million) reads per sample in most RNAseq studies at the time. Under such state of the art at the time of filing, the TL1A short isoform transcripts would have been missed in these typical RNAseq studies.

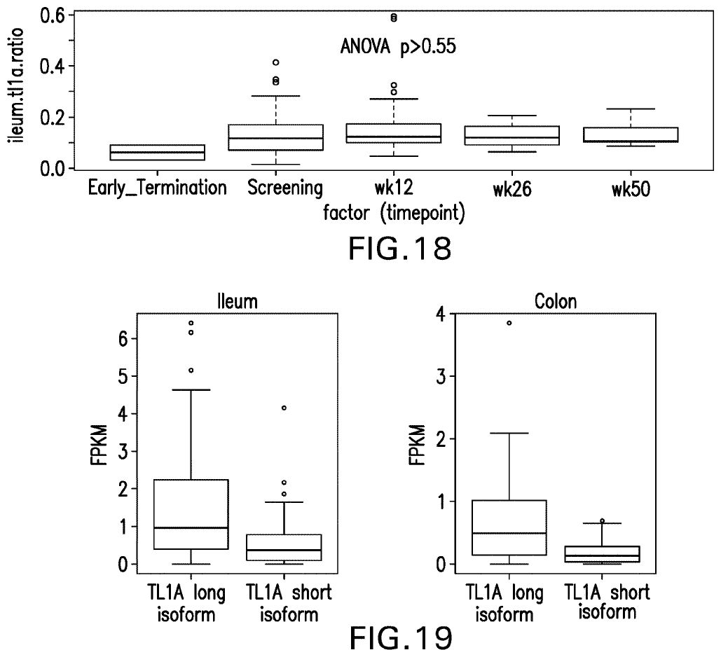

Having detected both TL1A long and short isoforms in IBD patients' tissues, the inventors analyzed whether the expression of TL1A isoforms can be detected across the time points during the course of treatment and whether the ratio between the TL1A short and long isoforms changes during the course of treatment. RNAseq was thus performed with biopsy samples collected during the course of TL1A inhibitor treatment for CD patients. As shown in FIG. 18, both TL1A short and long isoforms were detected in ileum across the course of treatment and no statistically significant changes were seen in the ratio between TL1A short isoform and TL1A long isoform across the time points during the treatment.

Next, the inventors analyzed samples from an independent cohort to validate the observed expression patterns of TL1A isoforms. As shown in FIG. 19, in samples of an IBD patient cohort treated and collected at University of California San Diego, both TL1A short and long isoforms were detected in ileum and colon tissues and the expression of both TL1A short and long isoforms were higher in ileum. Thus, the expression of both TL1A short and long isoforms in ileum and colon of IBD patients and the higher expression of both TL1A short and long isoforms in ileum compared to colon are established and validated in at least two independent patient cohorts.

### 7.12 Example 12: Association of TL1A Isoform Ratio and Patients' Response to Treatment with TL1A Inhibitors

Having shown both TL1A long and short isoforms express in gastrointestinal tissues in IBD patients, the inventors analyzed whether the ratio between TL1A short isoform and TL1A long isoform is associated with the IBD patients' response to the TL1A inhibitor treatment. Briefly, the CD patients were recruited, treated with anti-TL1A antibody A219, and biopsied to obtain gastrointestinal tissues, as described in clinical trials in Examples above. RNAseq was performed to determine the ratio between TL1A short isoform and TL1A long isoform based on the level of mRNA transcripts observed in the RANseq. The resulting TL1A isoform ratio was then analyzed for statistical association with these CD patients' endoscopic response to the anti-TL1A treatment.

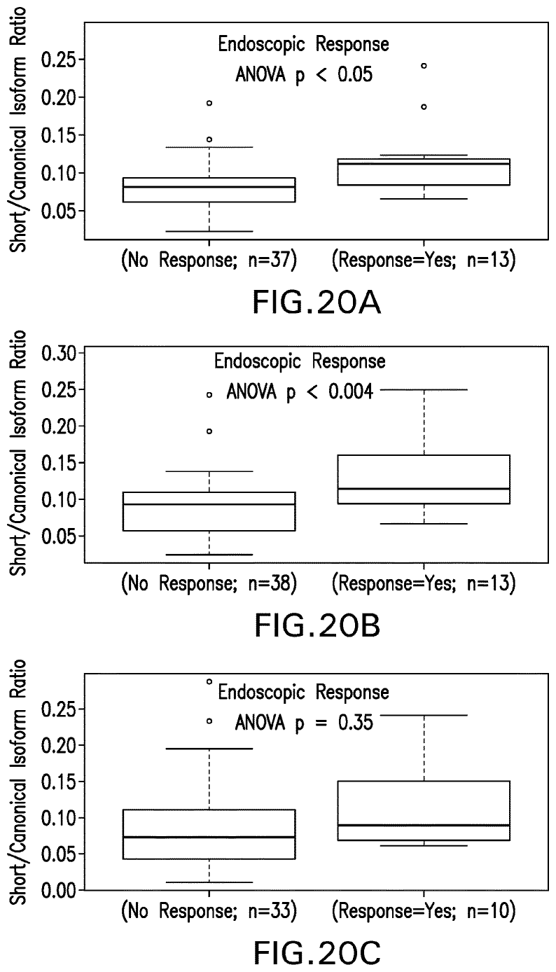

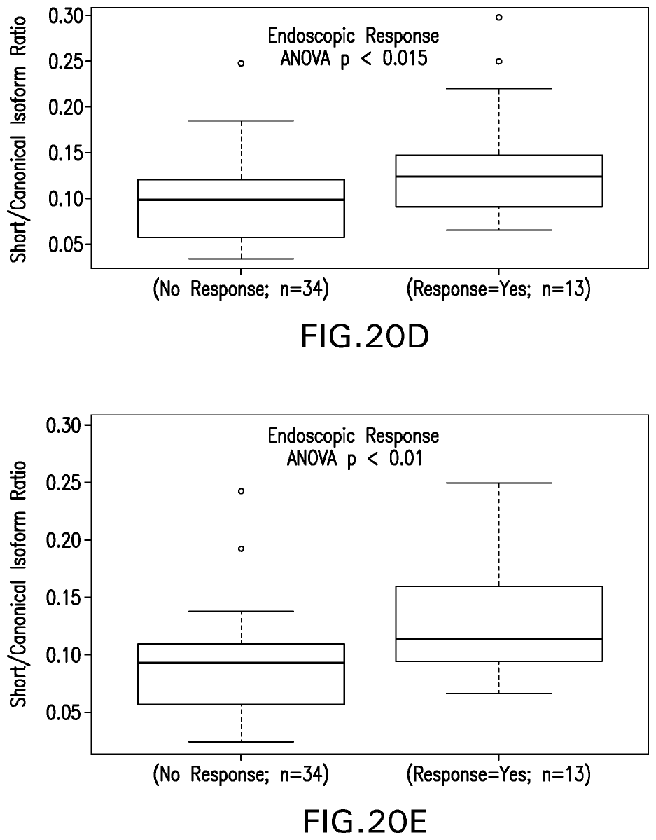

As shown in FIG. 20A and FIG. 20B, CD patients who responded to the treatment by anti-TL1A antibody (A219 antibody) had a higher TL1A short to long isoform ratio. The association between the TL1A isoform ratio and CD patients endoscopic response is statistically significant, with a p-value less than 0.05. In FIG. 20A, a combination of available baseline (before treatment) and after treatment ileal samples were used to determine TL1A isoform ratio. Such combination of samples is support by FIG. 18, which shows that the TL1A isoform ratio remains stable across the time points from pre-treatment to the course of treatment. Alternatively, in FIG. 20B, a combination of available baseline-only ileal and colon samples was used to determine TL1A isoform ratio, as ileum and colon both express TL1A long and short isoforms. FIG. 20A and FIG. 20B together established a statistically significant association between the TL1A isoform ratio and CD patients' endoscopic response to anti-TL1A (A219) treatment. Furthermore, when only baseline biopsy tissues from the same kind of organ were used, a similar association between the TL1A isoform ratio and CD patients' endoscopic response to anti-TL1A treatment was established, although the samples size (n) and p value varied due to the availability of samples (see FIG. 20C for baseline ileal sample only, FIG. 20D for baseline colon sample only, and FIG. 20E for baseline rectum sample only)

Therefore, the disclosure established the association between TL1A short/long isoform ratio and CD patients' endoscopic response to TL1A inhibitor treatment.

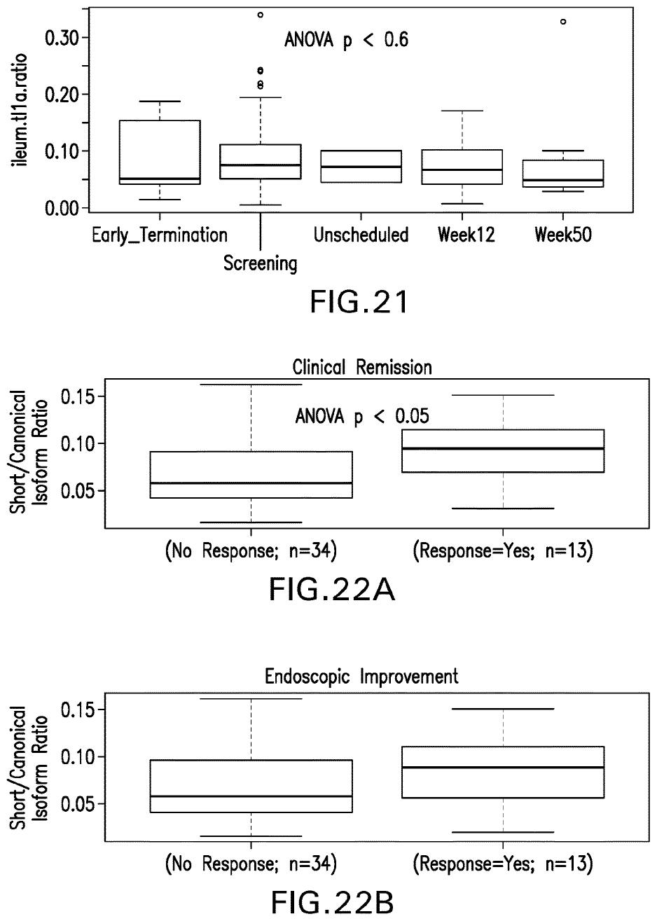

Having established the association between TL1A short/long isoform ratio and endoscopic response to TL1A inhibitor treatment in CD patients, inventors studied such potential association in UC patients. Briefly, UC patients were recruited, treated with anti-TL1A antibody A219, and biopsied to obtain gastrointestinal tissues, as described in clinical trials in Examples above. RNAseq was performed to determine the ratio between TL1A short isoform and TL1A long isoform based on the level of mRNA transcripts observed in the RANseq. The resulting TL1A isoform ratio was then analyzed for statistical association with UC patients' clinical remission or endoscopic improvement to the anti-TL1A treatment. As shown in FIG. 21, both TL1A short and long isoforms were detected in colon across the course of treatment and no statistically significant changes were seen in the ratio between TL1A short isoform and TL1A long isoform across the time points during the treatment with anti-TL1A (A219) in UC patients.

Next, the TL1A isoform ratio was then analyzed for statistical association with UC patients' endoscopic improvement or clinical remission to the anti-TL1A treatment. As shown in FIG. 22A, UC patients who experienced clinical remission to the treatment by anti-TL1A antibody (A219 antibody) had a higher TL1A short to long isoform ratio. Such trend of association was confirmed between TL1A short/long isoform ratio and UC patients' endoscopic improvement (see FIG. 22B). Furthermore, the association between the TL1A isoform ratio and UC patients' clinical remission is statistically significant, with a p-value less than 0.05 (FIG. 22A).

Therefore, the statistically significant association between TL1A short/long isoform ratio and patients' positive therapeutic response to anti-TL1A treatment was established and validated in both CD and UC patients. Accordingly, the disclosure established TL1A short/long isoform ratio as a CDx for selecting CD and/or UC patients having a positive therapeutic response to anti-TL1A treatment.

### 7.13 Example 13: Establishing a Proxy Marker for TL1A Short/Long Isoform Ratio

Having established the association between TL1A short/long isoform ratio and IBD patients' (e.g. UC and CD patients') therapeutic response to anti-TL1A treatment, the inventors sought to find exemplary proxy markers that have statistically significant correlation with TL1A short/long isoform ratio. Such proxy markers can be used in lieu of or in combination with the TL1A short/long isoform ratio to select IBD (e.g. UC or CD) patients who can have a positive therapeutic response to anti-TL1A treatment.

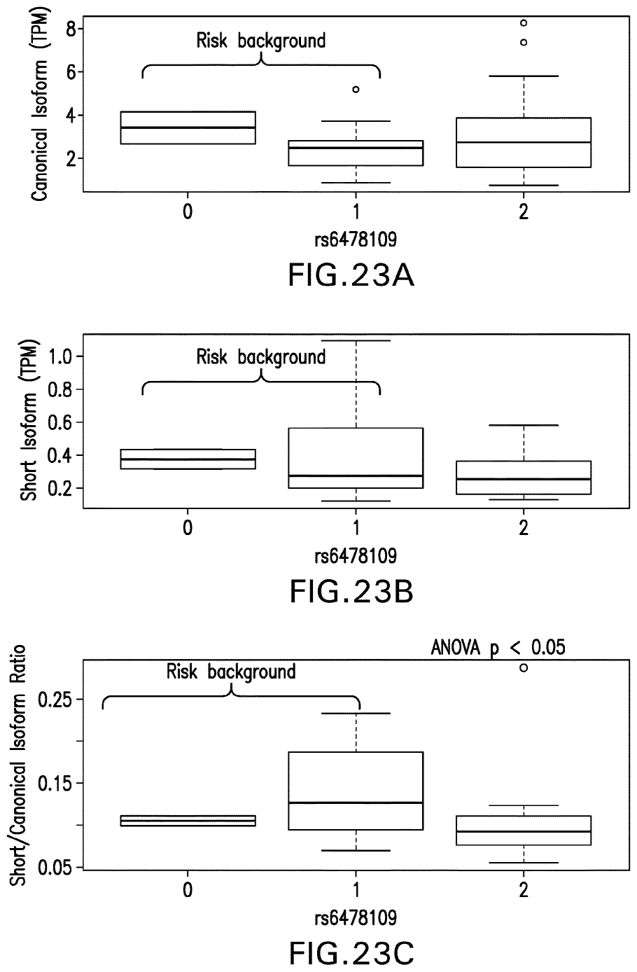

Various markers were determined in CD and UC patient cohort described above in the study of the association between TL1A short/long isoform ratio and patients' therapeutic response to anti-TL1A treatment. The correlation between the TL1A short/long isoform ratio and the proxy marker in such patient cohorts were determined. As shown in FIGS. 23A-23C, the risk alleles for rs6478109 SNP correlated with TL1A short/long isoform ratio in the cohort of CD patients with statistical significance (p<0.05). Therefore, the rs6478109 SNP is a validated proxy marker of TL1A short/long isoform ratio that can be used to select patients with positive therapeutic response to anti-TL1A treatment. Accordingly, the disclosure established rs6478109 SNP as a CDx for selecting CD and/or UC patients having a positive therapeutic response to anti-TL1A treatment.

### 7.14 Example 14: Validating the Performance of TL1A Isoform Ratio as CDx for Selecting Patients that have a Positive Therapeutic Response to Anti-TL1A Treatment

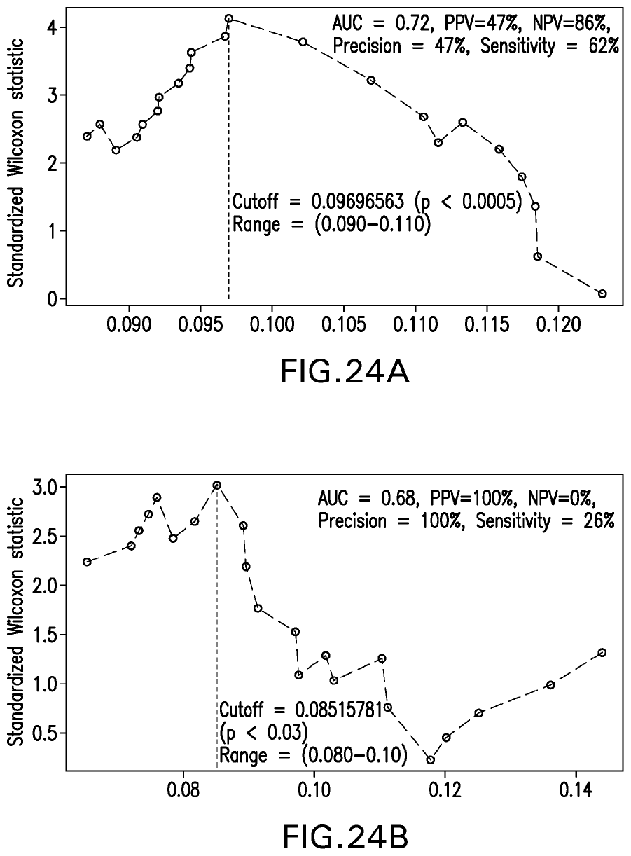

Having established the association between TL1A short/long isoform ratio and IBD patients' (e.g. UC and CD patients') therapeutic response to anti-TL1A treatment, the inventors sought to validate CDx based on TL1A short/long isoform ratio in selecting patients having a positive therapeutic response to ant-TL1A treatment. Based on the association studies in Section 7.12 and based on the standardized Wilcoxon statistic plot with TL1A isoform ratio and clinical endoscopic response (FIG. 24A), the cutoff for the TL1A short/long isoform ratio in the range of 0.090-0.110 could provide robust separation between CD patients who have a positive therapeutic response (endoscopic response) and those who don't (e.g. a higher percentage of CD patients with TL1A short/long isoform ratio ≥cutoff in the range of (0.090-0.110) would have positive therapeutic response to anti-TL1A (A219) treatment; in comparison, a lower percentage of CD patients with TL1A short/long isoform ratio <cutoff in the range of (0.090-0.110) would have positive therapeutic response to anti-TL1A (A219) treatment).

To further validate the performance of CDx based on TL1A isoform ratio, clinical trial results with CD patients as described above in Sections 7.10 and 7.12 were analyzed with a TL1A isoform ratio cutoff of 0.09696563, within the range of (0,090-0.110), indicated in the previous paragraph. Such specific TL1A isoform ratio cutoff was determined based on the standardized Wilcoxon statistic with TL1A isoform ratio and endoscopic response in CD patients observed in clinical trials of Sections 7.10 and 7.12 shown in FIG. 24A. Specifically, CD patients having TL1A isoform ratio >0.09696563 was determined as CDx positive and more likely to have a positive therapeutic response to anti-TL1A treatment, and CD patients having TL1A isoform ratio <0.09696563 was determined as CDx negative and less likely to have a positive therapeutic response to anti-TL1A treatment. Such a CDx model based on TL1A isoform ratio with 0.09696563 cutoff had a robust performance in selecting CD patients having a positive therapeutic response, with PPV=47%, NPV=86%, Precision=47%, Sensitivity=62%, and AUC=0.72. Additionally, the TL1A isoform ratio cutoff of 0.09696563 had a statistically significant p value of <0.0005, as shown in FIG. 24A.

Similarly, CDx based on TL1A isoform ratios were also validated in UC patients. Based on the association studies in Section 7.12 and based on the standardized Wilcoxon statistic plot with TL1A isoform ratio and clinical remission (FIG. 24B), the cutoff for the TL1A short/long isoform ratio in the range of 0.080-0.10 could provide robust separation between UC patients who have a positive therapeutic response (clinical remission) and those who don't (e.g. a higher percentage of UC patients with TL1A short/long isoform ratio≥cutoff in the range of (0.080-0.10) would have a positive therapeutic response to anti-TL1A (A219) treatment; in comparison, a lower percentage of UC patients with TL1A short/long isoform ratio<cutoff in the range of (0.080-0.10) would have a positive therapeutic response to anti-TL1A (A219) treatment).

To further validate the performance of CDx based on TL1A isoform ratio, clinical trial results with UC patients as described above in Sections 7.9 and 7.12 were analyzed with TL1A isoform ratio and a cutoff of 0.08515781, within the range of (0.080-0.10), indicated in the previous paragraph. Such specific TL1A isoform ratio cutoff was determined based on the standardized Wilcoxon statistic with TL1A isoform ratio and clinical remission in UC patients observed in clinical trials of Sections 7.9 and 7.12 shown in FIG. 24B. Specifically, UC patients having TL1A isoform ratio≥0.08515781 was determined as CDx positive and more likely to have a positive therapeutic response (clinical remission) to anti-TL1A treatment, and UC patients having TL1A isoform ratio<0.08515781 was determined as CDx negative and less likely to have a positive therapeutic response (clinical remission) to anti-TL1A treatment. Such a CDx model based on TL1A isoform ratio with 0.08515781 cutoff had a robust performance in selecting UC patients having a positive therapeutic response, with AUC=0.68, PPV=100%. NPV=0%, Precision=100%, Sensitivity=26%. Additionally, the TL1A isoform ratio cutoff of 0.08515781 had a statistically significant p value of <0.03, as shown in FIG. 24B.

The TL1A isoform ratio in this Example is calculated as ln(1+TL1Aₛₕₒᵣₜ/TL1A_{long}).

Accordingly, TL1A isoform ratio has been clinically validated as a CDx for selecting IBD patients (including CD patients and UC patients) who have a positive therapeutic response to treatment with inhibitors of TL1A expression or activity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a ratio between TL1A short isoform and TL1A long isoform (“TL1A isoform ratio”), a proxy marker having a statistically significant correlation with the TL1A isoform ratio, or a combination thereof.

2. (canceled)

3. A method of determining a ratio between TL1A short isoform and TL1A long isoform (“TL1A isoform ratio”) or a proxy marker having a statistically significant correlation with the TL1A isoform ratio for a subject with an inflammatory, a fibrotic, or a fibrostenotic disease or condition, the method comprising:
(a) obtaining or having obtained a sample from the subject;
(b) subjecting the sample to an assay adapted to detect an expression level of the TL1A short isoform and an expression level of the TL1A long isoform; and
(c) determining a ratio TL1A isoform ratio, a proxy marker having a statistically significant correlation with the TL1A isoform ratio, or a combination thereof, wherein whether the subject is suitable for treatment with an inhibitor of TL1A activity or expression is indicated based on the TL1A isoform ratio or the proxy marker.

4-6. (canceled)

7. The method of claim 1, wherein the correlation is Pearson correlation or Spearman correlation, optionally wherein the correlation has a correlation coefficient with an absolute value of at least about 0.30, at least about 0.31, at least about 0.32, at least about 0.33, at least about 0.34, at least about 0.35, at least about 0.36, at least about 0.37, at least about 0.38, at least about 0.39, at least about 0.40, at least about 0.41, at least about 0.42, at least about 0.43, at least about 0.44, at least about 0.45, at least about 0.46, at least about 0.47, at least about 0.48, at least about 0.49, at least about 0.50, at least about 0.51, at least about 0.52, at least about 0.53, at least about 0.54, at least about 0.55, at least about 0.56, at least about 0.57, at least about 0.58, at least about 0.59, at least about 0.60, at least about 0.61, at least about 0.62, at least about 0.63, at least about 0.64, at least about 0.65, at least about 0.66, at least about 0.67, at least about 0.68, at least about 0.69, at least about 0.70, at least about 0.71, at least about 0.72, at least about 0.73, at least about 0.74, at least about 0.75, at least about 0.76, at least about 0.77, at least about 0.78, at least about 0.79, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or 1.

8. The method of claim 1, wherein the proxy marker for a ratio between TL1A short isoform and TL1A long isoform (“TL1A isoform ratio”) is identified by:
(a) determine the TL1A isoform ratio in a population of subjects;
(b) determining a marker other than the TL1A isoform ratio in the population of subjects;
(c) determining the correlation between the marker in (b) and the TL1A isoform ratio in the population; and
(d) identifying the marker from (b) as a proxy marker for the TL1A isoform ratio if the marker has a statistically significant correlation with the TL1A isoform ratio,
optionally wherein the proxy marker relative to a cutoff is indicative that the subject is suitable for treatment with an inhibitor of TL1A activity or expression for treatment of the inflammatory, fibrotic, or fibrostenotic disease or condition.

9-10. (canceled)

11. A computer-implemented method of determining a ratio between TL1A short isoform and TL1A long isoform (“TL1A isoform ratio”) for a subject, the method comprising:
(a) obtaining expression data in a sample from the subject;
(b) determining expression of TL1A short isoform and TL1A long isoform;
(c) calculating the TL1A isoform ratio; and
(d) applying a cutoff to the TL1A isoform ratio, wherein the TL1A isoform ratio relative to the cutoff is indicative that the subject is suitable for treatment with an inhibitor of TL1A activity or expression for treatment of the inflammatory, fibrotic, or fibrostenotic disease or condition, optionally wherein the TL1A isoform ratio relative to the cutoff is the TL1A isoform ratio above the cutoff.

12-13. (canceled)

14. The method of claim 1, wherein the proxy marker comprises rs6478109.

15. The method of claim 1, wherein based on the proxy marker is (i) based on the proxy marker above a cutoff if the proxy marker and the TL1A isoform ratio has a positive correlation (such proxy marker as “positive proxy marker”); or (ii) based on the proxy marker below a cutoff if the proxy marker and the TL1A isoform ratio has a negative correlation (such proxy marker as “inverse proxy marker”),
optionally wherein TL1A isoform ratio or a positive proxy marker above a cutoff is determined if an inverse-proxy marker is below an alternative cutoff, wherein the inverse proxy marker has a negative correlation with the TL1A isoform ratio,
optionally wherein inverse proxy marker comprises the ratio of TL1A long isoform to the TL1A short isoform,
optionally wherein TL1A isoform ratio above a cutoff is determined if the ratio of TL1A long isoform to the TL1A short isoform is below an alternative cutoff, optionally wherein the cutoff ranges from about 0.080 to about 0.110, optionally wherein the cutoff is about wherein the cutoff is about 0.085, optionally wherein the cutoff is about 0.097,
optionally wherein the cutoff is for selecting CD or UC patients,
optionally wherein the cutoff is (i) the TL1A isoform ratio or proxy marker level in a heathy subject without the inflammatory, fibrotic, or fibrostenotic disease or condition or (ii) the TL1A isoform ratio or proxy marker level in a tissue not afflicted by the inflammatory, fibrotic, or fibrostenotic disease or condition,
optionally wherein the TL1A isoform ratio is calculated as, TL1Aₛₕₒᵣₜ/TL1A_{long}, ln(TL1Aₛₕₒᵣₜ/TL1A_{long}), or ln(TL1Aₛₕₒᵣₜ/TL1A_{long}+n), wherein TL1Aₛₕₒᵣₜ is an expression level of the TL1A short isoform, TL1A_{long} is an expression level of the TL1Along isoform, and n is a positive number, optionally wherein n is 1, and
optionally (i) the expression level of the TL1A short isoform is the level of gene expression of the TL1A short isoform and the expression level of the TL1A long isoform is the level of gene expression of the TL1A long isoform; (ii) the expression level of the TL1A short isoform is the level of protein expression of the TL1A short isoform and the expression level of the TL1Along isoform is the level of protein expression of the TL1A long isoform; (iii) the expression level of the TL1A short isoform is the level of gene expression of the TL1A short isoform and the expression level of the TL1A long isoform is the level of protein expression of the TL1A long isoform; or (iv) the expression level of the TL1A short isoform is the level of protein expression of the TL1A short isoform and the expression level of the TL1A long isoform is the level of gene expression of the TL1A long isoform.

16-30. (canceled)

31. The method of claim 1, wherein the TL1A isoform ratio or the proxy marker is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%,
optionally wherein the TL1A isoform ratio or the proxy marker is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%,
optionally wherein the TL1A isoform ratio or the proxy marker is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%,
optionally wherein the TL1A isoform ratio or the proxy marker is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%,
optionally wherein the cutoff is such that the TL1A isoform ratio or the proxy marker above the cutoff is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%,
optionally wherein the cutoff is such that the TL1A isoform ratio or the proxy marker above the cutoff is predictive of a positive therapeutic response in the subject to a treatment with the inhibitor of TL1A activity or expression with a specificity of at least about 50%, 55%, 60%, 65%, 70%, 75%, 80%, 85%, 90%, or 95%,
optionally wherein the cutoff is such that the TL1A isoform ratio or the proxy marker above the cutoff is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%,
optionally wherein the cutoff is such that the TL1A isoform ratio or the proxy marker above the cutoff is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%,
optionally wherein the cutoff is such that the TL1A isoform ratio or the proxy marker above the cutoff is predictive of an increase of one or more IBD enriched cell types with a positive rate of at least about 10%, 15%, 20%, 25%, 30%, 35%, 40%, 45%, 50%, 55%, 60%, 65%, 70%, or 75%, and
optionally wherein the cutoff is such that the TL1A isoform ratio or the proxy marker above the cutoff is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%.

32-42. (canceled)

43. The method of claim 1, wherein the TL1A isoform ratio is TL1A isoform ratio in a diseased tissue afflicted by the inflammatory, fibrotic, or fibrostenotic disease or condition,
optionally wherein the diseased tissue is selected from the group consisting of ileum, colon, rectum, or small intestine,
optionally wherein the TL1A isoform ratio is TL1A isoform ratio in a cell contributory to the inflammatory, fibrotic, or fibrostenotic disease or condition, and
optionally wherein the cell is selected from the group consisting of a Goblet cell, a pericyte, a smooth muscle cell, an enterocyte, a clonocyte, a monocyte-derived dendritic cells (moDC), a resident macrophage, or a colon transit-amplifying (TA) cell.

44-46. (canceled)

47. The method of claim 3, wherein the TL1A isoform ratio is TL1A isoform ratio in blood of the subject.

48-60. (canceled)

61. The method of claim 1, wherein the statistically significant correlation is a correlation with p value less than 0.1, 0.09, 0.08, 0.07, 0.06, 0.05, 0.04, 0.03, 0.02, or 0.01.

62. The method of claim 1, wherein the subject has been treated or has not been treated with an advanced IBD therapy prior to the treatment with the inhibitor of TL1A activity or expression,
optionally wherein the advanced IBD therapy comprises one or more selected from the group consisting of a biologic therapeutic agent for IBD, an SIP1 modulator, or a JAK inhibitor, and
optionally wherein the biologic therapeutic agent for IBD comprises an anti-TNFα antibody, an anti-IL23 antibody, or an anti-integrin α4β7 antibody.

63-65. (canceled)

66. The method of claim 1, wherein the inhibitor of TL1A activity or expression is an antibody or antigen binding fragment thereof that binds to TL1A (anti-TL1A antibody or antigen binding fragment), wherein the anti-TL1A antibody or antigen binding fragment comprises a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and a light chain variable region comprising: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15,
optionally wherein the inhibitor of TL1A activity or expression is an anti-TL1A antibody or antigen binding fragment, wherein the anti-TL1A antibody or antigen binding fragment comprises a heavy chain variable domain comprising an amino acid sequence at least about 90% identical to any one of SEQ ID NOS: 101-135, or 310-302, and a light chain variable domain comprising an amino acid sequence at least about 90% identical to any one of SEQ ID NOS: 201-206 or 303,
optionally wherein the anti-TL1A antibody or antigen binding fragment comprises: (a) a heavy chain variable framework region comprising a human IGHV1-46*02 framework or a modified human IGHV1-46*02 framework; and (b) a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework,
optionally wherein an amino acid modification of the less than 14 amino acid modifications comprises: (a) the amino acid modification is at position 47 in the heavy chain variable region, and the amino acid at position 47 is R, N, D, C, Q, E, G, H, I, L, K, M, F, P, S, T, W, Y, or V: (b) the amino acid modification is at position 45 in the heavy chain variable region, and the amino acid at position 45 is A, N, D, C, Q, E, G, H, I, L, K, M, F, P, S, T, W, Y, or V; (c) the amino acid modification is at position 55 in the heavy chain variable region, and the amino acid at position 55 is A, R, N, D, C, Q, E, G, H, I, L, K, F, P, S, T, W, Y, or V; (d) the amino acid modification is at position 78 in the heavy chain variable region, and the amino acid at position 78 is A, R, N, D, C, Q, E, G, H, I, L, K, M, F, P, S, T, W, or Y; (e) the amino acid modification is at position 80 in the heavy chain variable region, and the amino acid at position 80 is A, R, N, D, C, Q, E, G, H, I, L, K, F, P, S, T, W, Y, or V; (f) the amino acid modification is at position 82 in the heavy chain variable region, and the amino acid at position 82 is A, N, D, C, Q, E, G, H, I, L, K, M, F, P, S, T, W, Y, or V: (g) the amino acid modification is at position 89 in the heavy chain variable region, and the amino acid at position 89 is A, R, N, D, C, Q, E, G, H, I, L, K, M, F, P, S, T, W, or Y; or (h) the amino acid modification is at position 91 in the heavy chain variable region, and the amino acid at position 91 is A, R, N, D, C, Q, E, G, H, I, L, K, F, P, S, T, W, Y, or V; or a combination of two or more modifications selected from (a) to (h),
optionally wherein an amino acid modification of the less than 14 amino acid modifications comprises: A47R, R45K, M55I, V78A, M80I, R82T, V89A, M91L in the heavy chain variable region, per Aho or Kabat numbering,
optionally wherein an amino acid modification of the less than 14 amino acid modifications comprises: (a) a modification at amino acid position 54 in the light chain variable region; and/or (b) a modification at amino acid position 55 in the light chain variable region; per Aho or Kabat numbering,
optionally wherein an amino acid modification of the less than 14 amino acid modifications comprises: (a) the amino acid modification is at position 54 of the light chain variable region, and the amino acid at position 54 is A, R, N, D, C, Q, E, G, H, I, K, M, F, P, S, T, W, Y, or V; and/or (b) the amino acid modification is at position 55 of the light chain variable region, and the amino acid at position 55 is A, R, N, D, C, Q, E, G, H, I, K, M, F, P, S, T, W, Y, or V, and
optionally wherein an amino acid modification of the less than 14 amino acid modifications comprises L54P and/or L55 W in the light chain variable region, per Aho or Kabat numbering.

67-75. (canceled)

76. The method of claim 1, wherein the inhibitor of TL1A activity or expression is an antibody or antigen binding fragment thereof that binds to TL1A and comprises:
1 a heavy chain variable region comprising SEQ ID NO: 301 X1VQLVQSGAEVKKPGASVKVSCKAS[HCDR1]WVX2QX3PGQGLEWX4G[HCDR2]RX5 TX6TX7DTSTSTX8YX9ELSSLRSEDTAVYYCAR[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRFSG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V,
or a heavy chain variable region comprising SEQ ID NO: 302 X1VOLVOSGAEVKKPGASVKVSCKAS[HCDR1]WVX2QX3PGQGLEWX4G[HCDR2]RX5 TX6TX7DTSTSTX8YX9ELSSLRSEDTAVYYC[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRESG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V,
optionally wherein:
(A) X1 is Q or E;
(B) X2 is R or K
(C) X3 is A or R;
(D) X4 is M or I;
(E) X5 is V or A;
(F) X6 is M or I;
(G) X7 is R or T;
(H) X8 is V or A;
(I) X9 is M or L
(J) X10 is L or P;
(K) X11 is L or W; or
(L) X1-X11 are any combination of (A) to (K),
optionally wherein the antibody or antigen binding fragment comprises a heavy chain CDR1 as set forth by SEQ ID NO: 1, a heavy chain CDR2 as set forth by any one of SEQ ID NOS: 2-5, a heavy chain CDR3 as set forth by any one of SEQ ID NOS: 6-9, a light chain CDR1 as set forth by SEQ ID NO: 10, a light chain CDR2 as set forth by SEQ ID NO: 11, and a light chain CDR3 as set forth by any one of SEQ ID NOS: 12-15,
optionally wherein the antibody or antigen binding fragment comprises a heavy chain framework (FR) 1 as set forth by SEQ ID NO: 304, a heavy chain FR2 as set forth by SEQ ID NO: 305 or SEQ ID NO: 313, a heavy chain FR3 as set forth by any one of SEQ ID NOS: 306, 307, 314, or 315, a heavy chain FR4 as set forth by SEQ ID NO: 308, a light chain FR1 as set forth by SEQ ID NO: 309, a light chain FR2 as set forth by SEQ ID NO: 310, a light chain FR3 as set forth by SEQ ID NO: 311, or a light chain FR4 as set forth by SEQ ID NO: 312, or a combination thereof,
optionally wherein the antibody or antigen binding fragment comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (i) 329A, 329G, 329Y, or 329R (k) 331S, (l) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (g) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376 W, or 376Y, (11) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgGlo), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (111) A330L, (mmm) P331A or P331S, or (nnn) any combination of two or more selected from (a)-(uu), per Kabat numbering,
optionally wherein the antibody or antigen binding fragment comprises a human IgG4 Fc region,
optionally wherein the antibody or antigen binding fragment comprises a Fc region comprising a sequence at least about 90%, 91%, 92%, 93%, 94%, 95%, 96%, 97%, 98%, 99%, or 100% identical to any one of SEQ ID NOS: 320-362
optionally wherein the antibody of antigen binding fragment comprises a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1,
optionally wherein the antibody or antigen binding fragment comprises a Fc region and wherein the Fc comprises the human IgG1 comprises SEQ ID NO: 320,
optionally wherein the antibody or antigen binding fragment comprises a Fc region and wherein the ADCC function of the Fc region comprising reduced ADCC is at least about 50% reduced as compared to human IgG1,
optionally wherein the antibody or antigen binding fragment comprises a Fc region and wherein the CDC function of the Fc region comprising reduced CDC is at least about 50% reduced as compared to human IgG1,
optionally wherein the antibody or antigen binding fragment comprises a Fc region and wherein the Fc comprises (i) a human IgG4 Fc region or (ii) a human IgG4 Fc region comprising (a) S228P, (b) S228P and L235E, or (c) S228P, F234A, and L235A, per Kabat numbering,
optionally wherein the antibody or antigen binding fragment comprises a Fc region and wherein the Fc comprises a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2G),
optionally wherein the antibody or antigen binding fragment comprises a Fc region and wherein the Fc comprises a human IgG1 with a substitution selected from 329A, 329G, 329Y, 331S, 236F, 236R, 238A, 238E, 238G, 238H, 238I, 238V, 238 W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering,
optionally wherein the antibody or antigen binding fragment comprises a Fc region and wherein the Fc comprises a sequence at least about 90%, 91%, 92%, 93%, 94%, 95%, 96%, 97%, 98%, 99%, or 100% identical to any one of SEQ ID NOS: 320-362,
optionally wherein the antibody or antigen binding fragment comprises a Fc region and wherein the Fc comprises any one of SEQ ID NOs: 401-413 or a sequence at least about 90%, 91%, 92%, 93%, 94%, 95%, 96%, 97%, 98%, or 99% identical to any one of SEQ ID NOs: 401-413,
optionally wherein the antibody or antigen binding fragment comprises a heavy chain comprising any one of SEQ ID NOs: 501-513 or a sequence at least about 90%, 91%, 92%, 93%, 94%, 95%, 96%, 97%, 98%, or 99% identical to any one of SEQ ID NOs: 501-513, and
optionally wherein the antibody or antigen binding fragment comprises a light chain comprising any one of SEQ ID NO: 514 or a sequence at least about 90%, 91%, 92%, 93%, 94%, 95%, 96%, 97%, 98%, or 99% identical to any one of SEQ 515.

77-94. (canceled)

95. The method of claim 1, wherein the inflammatory, fibrotic, or fibrostenotic disease or condition comprises inflammatory bowel disease, Crohn's disease, obstructive Crohn's disease, ulcerative colitis, intestinal fibrosis, intestinal fibrostenosis, rheumatoid arthritis, or primary sclerosing cholangitis,
optionally wherein the Crohn's disease is ileal, ileocolonic, or colonic Crohn's disease, and
optionally wherein the subject has, or is at risk for developing, a non-response or loss-of-response to a standard therapy comprising glucocorticosteriods, anti-TNF therapy, anti-α4β7 therapy, anti-IL12p40 therapy, or a combination thereof.

96-97. (canceled)

98. The method of claim 47, wherein the expression level of the TL1A short isoform and the expression level of the TL1A long isoform is determined by ELISA, RNA sequencing, PCR, or mass spectrometry.


SEQUENCE LISTING (3320 sequences)

SEQ ID NO: 1 - protein, 10 aa, synthetic construct
GFDIQDTYMH

SEQ ID NO: 2 - protein, 17 aa, synthetic construct
RIDPASGHTKYDPKFQV

SEQ ID NO: 3 - protein, 17 aa, synthetic construct
RIEPASGHIKYDPKFQG

SEQ ID NO: 4 - protein, 17 aa, synthetic construct
RIDPASGHIKYDPKFQG

SEQ ID NO: 5 - protein, 17 aa, synthetic construct
RIEPASGHIKYDPKFQV

SEQ ID NO: 6 - protein, 7 aa, synthetic construct
SGGLPDV

SEQ ID NO: 7 - protein, 9 aa, synthetic construct
ARSGGLPDV

SEQ ID NO: 8 - protein, 7 aa, synthetic construct
SGGLPDW

SEQ ID NO: 9 - protein, 9 aa, synthetic construct
ARSGGLPDW

SEQ ID NO: 10 - protein, 10 aa, synthetic construct
RASSSVSYMY

SEQ ID NO: 11 - protein, 7 aa, synthetic construct
ATSNLAS

SEQ ID NO: 12 - protein, 9 aa, synthetic construct
QQWEGNPRT

SEQ ID NO: 13 - protein, 9 aa, synthetic construct
QQWKGNPRT

SEQ ID NO: 14 - protein, 9 aa, synthetic construct
QQWSGNPRT

SEQ ID NO: 15 - protein, 9 aa, synthetic construct
QQWSRNPRT

SEQ ID NO: 16-100 - skipped (85 entries)

SEQ ID NO: 101 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTVYMELSSLRSEDTAVYYCARSGGLPDVWGQGTTVTVSS

SEQ ID NO: 102 - protein, 116 aa, synthetic construct
QVQLVQSGAEVKKPGASVKVSCKASGFDIQDTYMHWVKQRPGQGLEWMGRIDPASGHTKYDPKFQVRVTMTRDTSTSTAYLELSSLRSEDTAVYYCARSGGLPDVWGQGTTVTVSS

SEQ ID NO: 103 - protein, 116 aa, synthetic construct
EVQLVQSGAEVKKPGASVKVSCKASGFDIQDTYMHWVRQRPGQGLEWIGRIDPASGHTKYDPKFQVRATITTDTSTSTAYLELSSLRSEDTAVYYCARSGGLPDVWGQGTTVTVSS

SEQ ID NO: 104 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 105 - protein, 116 aa, synthetic construct
QVQLVQSGAEVKKPGASVKVSCKASGFDIQDTYMHWVKQRPGQGLEWMGRIDPASGHTKYDPKFQVRATITRDTSTSTAYLELSSLRSEDTAVYYCARSGGLPDVWGQGTTVTVSS

SEQ ID NO: 106 - protein, 116 aa, synthetic construct
QVQLVQSGAEVKKPGASVKVSCKASGFDIQDTYMHWVRQRPGQGLEWMGRIDPASGHTKYDPKFQVRVTITTDTSTSTVYMELSSLRSEDTAVYYCARSGGLPDVWGQGTTVTVSS

SEQ ID NO: 107 - protein, 116 aa, synthetic construct
QVQLVQSGAEVKKPGASVKVSCKASGFDIQDTYMHWVRQRPGQGLEWIGRIDPASGHTKYDPKFQVRATITTDTSTSTAYLELSSLRSEDTAVYYCARSGGLPDVWGQGTTVTVSS

SEQ ID NO: 108 - protein, 116 aa, synthetic construct
QVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 109 - protein, 116 aa, synthetic construct
EVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 110 - protein, 116 aa, synthetic construct
QVQLVQSGAEVKKPGASVKVSCKASGFDIQDTYMHWVRQRPGQGLEWMGRIDPASGHTKYDPKFQVRVTITTDTSTSTAYLELSSLRSEDTAVYYCARSGGLPDVWGQGTTVTVSS

SEQ ID NO: 111 - protein, 116 aa, synthetic construct
QVQLVQSGAEVKKPGASVKVSCKASGFDIQDTYMHWVRQRPGQGLEWMGRIDPASGHTKYDPKFQVRVTITRDTSTSTAYLELSSLRSEDTAVYYCARSGGLPDVWGQGTTVTVSS

SEQ ID NO: 112 - protein, 116 aa, synthetic construct
QVQLVQSGAEVKKPGASVKVSCKASGFDIQDTYMHWVRQRPGQGLEWIGRIDPASGHTKYDPKFQVRVTITRDTSTSTAYLELSSLRSEDTAVYYCARSGGLPDVWGQGTTVTVSS

SEQ ID NO: 113 - protein, 116 aa, synthetic construct
QVQLVQSGAEVKKPGASVKVSCKASGFDIQDTYMHWVRQRPGQGLEWIGRIDPASGHTKYDPKFQVRATITRDTSTSTAYLELSSLRSEDTAVYYCARSGGLPDVWGQGTTVTVSS

SEQ ID NO: 114 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTAYLELSSLRSEDTAVYYCARSGGLPDVWGQGTTVTVSS

SEQ ID NO: 115 - protein, 116 aa, synthetic construct
QVQLVQSGAEVKKPGASVKVSCKASGFDIQDTYMHWVKQRPGQGLEWIGRIDPASGHTKYDPKFQVRVTITTDTSTSTAYLELSSLRSEDTAVYYCARSGGLPDVWGQGTTVTVSS

SEQ ID NO: 116 - protein, 116 aa, synthetic construct
EVQLVQSGAEVKKPGASVKVSCKASGFDIQDTYMHWVKQAPGQGLEWMGRIEPASGHIKYDPKFQGRVTMTRDTSTSTVYMELSSLRSEDTAVYYCARSGGLPDWWGQGTTVTVSS

SEQ ID NO: 117 - protein, 116 aa, synthetic construct
QVQLVQSGAEVKKPGASVKVSCKASGFDIQDTYMHWVKQRPGQGLEWMGRIEPASGHIKYDPKFQGRVTMTRDTSTSTVYMELSSLRSEDTAVYYCARSGGLPDWWGQGTTVTVSS

SEQ ID NO: 118 - protein, 116 aa, synthetic construct
QVQLVQSGAEVKKPGASVKVSCKASGFDIQDTYMHWVKQRPGQGLEWMGRIDPASGHTKYDPKFQVRVTITTDTSTSTAYLELSSLRSEDTAVYYCARSGGLPDVWGQGTTVTVSS

SEQ ID NO: 119 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTAYMELSSLRSEDTAVYYCARSGGLPDVWGQGTTVTVSS

SEQ ID NO: 120 - protein, 116 aa, synthetic construct
QVQLVQSGAEVKKPGASVKVSCKASGFDIQDTYMHWVKQRPGQGLEWIGRIDPASGHTKYDPKFQVRVTITRDTSTSTAYLELSSLRSEDTAVYYCARSGGLPDVWGQGTTVTVSS

SEQ ID NO: 121 - protein, 116 aa, synthetic construct
QVQLVQSGAEVKKPGASVKVSCKASGFDIQDTYMHWVRQAPGQGLEWMGRIEPASGHIKYDPKFQVRVTMTRDTSTSTVYMELSSLRSEDTAVYYCARSGGLPDWWGQGTTVTVSS

SEQ ID NO: 122 - protein, 116 aa, synthetic construct
QVQLVQSGAEVKKPGASVKVSCKASGFDIQDTYMHWVRQAPGQGLEWMGRIEPASGHIKYDPKFQVRATMTRDTSTSTVYMELSSLRSEDTAVYYCARSGGLPDWWGQGTTVTVSS

SEQ ID NO: 123 - protein, 116 aa, synthetic construct
QVQLVQSGAEVKKPGASVKVSCKASGFDIQDTYMHWVRQAPGQGLEWMGRIEPASGHIKYDPKFQVRVTITRDTSTSTVYMELSSLRSEDTAVYYCARSGGLPDWWGQGTTVTVSS

SEQ ID NO: 124 - protein, 116 aa, synthetic construct
QVQLVQSGAEVKKPGASVKVSCKASGFDIQDTYMHWVRQAPGQGLEWMGRIEPASGHIKYDPKFQVRATITRDTSTSTVYMELSSLRSEDTAVYYCARSGGLPDWWGQGTTVTVSS

SEQ ID NO: 125 - protein, 116 aa, synthetic construct
QVQLVQSGAEVKKPGASVKVSCKASGFDIQDTYMHWVKQAPGQGLEWMGRIEPASGHIKYDPKFQGRVTMTRDTSTSTVYMELSSLRSEDTAVYYCARSGGLPDWWGQGTTVTVSS

SEQ ID NO: 126 - protein, 116 aa, synthetic construct
QVQLVQSGAEVKKPGASVKVSCKASGFDIQDTYMHWVKQAPGQGLEWMGRIDPASGHIKYDPKFQGRVTMTRDTSTSTVYMELSSLRSEDTAVYYCARSGGLPDWWGQGTTVTVSS

SEQ ID NO: 127 - protein, 116 aa, synthetic construct
QVQLVQSGAEVKKPGASVKVSCKASGFDIQDTYMHWVKQAPGQGLEWMGRIEPASGHIKYDPKFQGRATMTRDTSTSTVYMELSSLRSEDTAVYYCARSGGLPDWWGQGTTVTVSS

SEQ ID NO: 128 - protein, 116 aa, synthetic construct
QVQLVQSGAEVKKPGASVKVSCKASGFDIQDTYMHWVRQAPGQGLEWMGRIEPASGHIKYDPKFQGRATMTRDTSTSTVYMELSSLRSEDTAVYYCARSGGLPDWWGQGTTVTVSS

SEQ ID NO: 129 - protein, 116 aa, synthetic construct
EVQLVQSGAEVKKPGASVKVSCKASGFDIQDTYMHWVRQAPGQGLEWMGRIEPASGHIKYDPKFQGRATMTRDTSTSTVYMELSSLRSEDTAVYYCARSGGLPDWWGQGTTVTVSS

SEQ ID NO: 130 - protein, 116 aa, synthetic construct
QVQLVQSGAEVKKPGASVKVSCKASGFDIQDTYMHWVRQAPGQGLEWMGRIDPASGHIKYDPKFQGRATMTRDTSTSTVYMELSSLRSEDTAVYYCARSGGLPDWWGQGTTVTVSS

SEQ ID NO: 131 - protein, 116 aa, synthetic construct
QVQLVQSGAEVKKPGASVKVSCKASGFDIQDTYMHWVRQAPGQGLEWMGRIEPASGHIKYDPKFQGRATITTDTSTSTVYMELSSLRSEDTAVYYCARSGGLPDWWGQGTTVTVSS

SEQ ID NO: 132 - protein, 116 aa, synthetic construct
QVQLVQSGAEVKKPGASVKVSCKASGFDIQDTYMHWVRQAPGQGLEWMGRIEPASGHIKYDPKFQGRATMTRDTSTSTAYMELSSLRSEDTAVYYCARSGGLPDWWGQGTTVTVSS

SEQ ID NO: 133 - protein, 116 aa, synthetic construct
QVQLVQSGAEVKKPGASVKVSCKASGFDIQDTYMHWVRQAPGQGLEWMGRIEPASGHIKYDPKFQGRVTMTRDTSTSTAYMELSSLRSEDTAVYYCARSGGLPDWWGQGTTVTVSS

SEQ ID NO: 134 - protein, 116 aa, synthetic construct
EVQLVQSGAEVKKPGASVKVSCKASGFDIQDTYMHWVRQAPGQGLEWMGRIEPASGHIKYDPKFQGRVTMTRDTSTSTAYMELSSLRSEDTAVYYCARSGGLPDWWGQGTTVTVSS

SEQ ID NO: 135 - protein, 116 aa, synthetic construct
QVQLVQSGAEVKKPGASVKVSCKASGFDIQDTYMHWVRQAPGQGLEWMGRIDPASGHIKYDPKFQGRVTMTRDTSTSTAYMELSSLRSEDTAVYYCARSGGLPDWWGQGTTVTVSS

SEQ ID NO: 136 - protein, 116 aa, synthetic construct
QVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 137 - protein, 116 aa, synthetic construct
DVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 138 - protein, 116 aa, synthetic construct
QVQLVQSGAEVKKPGASVKVSCKASGFDIQDTYMHWVKQAPGQGLEWIGRIDPASGHTKYDPKFQVRATITTDTSTSTAYLELSSLRSEDTAVYYCARSGGLPDVWGQGTTVTVSS

SEQ ID NO: 139 - protein, 116 aa, synthetic construct
QVQLVQSGAEVKKPGASVKVSCKASGFDIQDTYMHWVRQAPGQGLEWIGRIDPASGHTKYDPKFQVRATITTDTSTSTAYLELSSLRSEDTAVYYCARSGGLPDVWGQGTTVTVSS

SEQ ID NO: 140 - protein, 116 aa, synthetic construct
QVQLVQSGAEVKKPGASVKVSCKASGFDIQDTYMHWVKQRPGQGLEWMGRIDPASGHTKYDPKFQVRATITTDTSTSTAYLELSSLRSEDTAVYYCARSGGLPDVWGQGTTVTVSS

SEQ ID NO: 141 - protein, 116 aa, synthetic construct
QVQLVQSGAEVKKPGASVKVSCKASGFDIQDTYMHWVKQRPGQGLEWIGRIDPASGHTKYDPKFQVRATITTDTSTSTVYLELSSLRSEDTAVYYCARSGGLPDVWGQGTTVTVSS

SEQ ID NO: 142 - protein, 116 aa, synthetic construct
QVQLVQSGAEVKKPGASVKVSCKASGFDIQDTYMHWVKQRPGQGLEWIGRIDPASGHTKYDPKFQVRATITTDTSTSTAYMELSSLRSEDTAVYYCARSGGLPDVWGQGTTVTVSS

SEQ ID NO: 143 - protein, 116 aa, synthetic construct
QVQLVQSGAEVKKPGASVKVSCKASGFDIQDTYMHWVKQRPGQGLEWIGRIDPASGHTKYDPKFQVRATITTDTSTSTVYMELSSLRSEDTAVYYCARSGGLPDVWGQGTTVTVSS

SEQ ID NO: 144 - protein, 116 aa, synthetic construct
QVQLVQSGAEVKKPGASVKVSCKASGFDIQDTYMHWVKQRPGQGLEWIGRIDPASGHTKYDPKFQVRATMTTDTSTSTAYLELSSLRSEDTAVYYCARSGGLPDVWGQGTTVTVSS

SEQ ID NO: 145 - protein, 116 aa, synthetic construct
QVQLVQSGAEVKKPGASVKVSCKASGFDIQDTYMHWVKQRPGQGLEWIGRIDPASGHTKYDPKFQVRATITRDTSTSTAYLELSSLRSEDTAVYYCARSGGLPDVWGQGTTVTVSS

SEQ ID NO: 146 - protein, 116 aa, synthetic construct
QVQLVQSGAEVKKPGASVKVSCKASGFDIQDTYMHWVKQRPGQGLEWIGRIDPASGHTKYDPKFQVRVTMTTDTSTSTAYLELSSLRSEDTAVYYCARSGGLPDVWGQGTTVTVSS

SEQ ID NO: 147 - protein, 116 aa, synthetic construct
QVQLVQSGAEVKKPGASVKVSCKASGFDIQDTYMHWVKQRPGQGLEWIGRIDPASGHTKYDPKFQVRATMTRDTSTSTAYLELSSLRSEDTAVYYCARSGGLPDVWGQGTTVTVSS

SEQ ID NO: 148 - protein, 116 aa, synthetic construct
QVQLVQSGAEVKKPGASVKVSCKASGFDIQDTYMHWVKQRPGQGLEWIGRIDPASGHTKYDPKFQVRVTMTRDTSTSTAYLELSSLRSEDTAVYYCARSGGLPDVWGQGTTVTVSS

SEQ ID NO: 149 - protein, 116 aa, synthetic construct
DVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 150 - protein, 116 aa, synthetic construct
QVQLVQSGAEVKKPGASVKVSCKASGFDIQDTYMHWVRQRPGQGLEWMGRIEPASGHIKYDPKFQGRVTMTRDTSTSTVYMELSSLRSEDTAVYYCARSGGLPDWWGQGTTVTVSS

SEQ ID NO: 151 - protein, 116 aa, synthetic construct
QVQLVQSGAEVKKPGASVKVSCKASGFDIQDTYMHWVRQAPGQGLEWMGRIDPASGHIKYDPKFQGRVTMTRDTSTSTVYMELSSLRSEDTAVYYCARSGGLPDWWGQGTTVTVSS

SEQ ID NO: 152 - protein, 116 aa, synthetic construct
QVQLVQSGAEVKKPGASVKVSCKASGFDIQDTYMHWVRQAPGQGLEWIGRIEPASGHIKYDPKFQGRVTMTRDTSTSTVYMELSSLRSEDTAVYYCARSGGLPDWWGQGTTVTVSS

SEQ ID NO: 153 - protein, 116 aa, synthetic construct
QVQLVQSGAEVKKPGASVKVSCKASGFDIQDTYMHWVRQAPGQGLEWIGRIDPASGHIKYDPKFQGRVTMTRDTSTSTVYMELSSLRSEDTAVYYCARSGGLPDWWGQGTTVTVSS

SEQ ID NO: 154 - protein, 116 aa, synthetic construct
QVQLVQSGAEVKKPGASVKVSCKASGFDIQDTYMHWVRQAPGQGLEWMGRIEPASGHVKYDPKFQGRVTMTRDTSTSTVYMELSSLRSEDTAVYYCARSGGLPDWWGQGTTVTVSS

SEQ ID NO: 155 - protein, 116 aa, synthetic construct
QVQLVQSGAEVKKPGASVKVSCKASGFDIQDTYMHWVRQAPGQGLEWMGRIEPASGHIKYDPKFQGRVTITRDTSTSTVYMELSSLRSEDTAVYYCARSGGLPDWWGQGTTVTVSS

SEQ ID NO: 156 - protein, 116 aa, synthetic construct
EVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 157 - protein, 116 aa, synthetic construct
QVQLVQSGAEVKKPGASVKVSCKASGFDIQDTYMHWVRQAPGQGLEWMGRIEPASGHIKYDPKFQGRVTMTTDTSTSTVYMELSSLRSEDTAVYYCARSGGLPDWWGQGTTVTVSS

SEQ ID NO: 158 - protein, 116 aa, synthetic construct
QVQLVQSGAEVKKPGASVKVSCKASGFDIQDTYMHWVRQAPGQGLEWMGRIEPASGHIKYDPKFQGRVTITTDTSTSTVYMELSSLRSEDTAVYYCARSGGLPDWWGQGTTVTVSS

SEQ ID NO: 159 - protein, 116 aa, synthetic construct
QVQLVQSGAEVKKPGASVKVSCKASGFDIQDTYMHWVRQAPGQGLEWMGRIEPASGHTKYDPKFQGRVTMTRDTSTSTVYMELSSLRSEDTAVYYCARSGGLPDWWGQGTTVTVSS

SEQ ID NO: 160 - protein, 116 aa, synthetic construct
QVQLVQSGAEVKKPGASVKVSCKASGFDIQDTYMHWVRQAPGQGLEWMGRIEPASGHIKYDPKFQGRATITRDTSTSTVYMELSSLRSEDTAVYYCARSGGLPDWWGQGTTVTVSS

SEQ ID NO: 161 - protein, 116 aa, synthetic construct
QVQLVQSGAEVKKPGASVKVSCKASGFDIQDTYMHWVRQAPGQGLEWMGRIEPASGHIKYDPKFQGRVTMTRDTSTSTVYLELSSLRSEDTAVYYCARSGGLPDWWGQGTTVTVSS

SEQ ID NO: 162 - protein, 116 aa, synthetic construct
QVQLVQSGAEVKKPGASVKVSCKASGFDIQDTYMHWVRQAPGQGLEWMGRIEPASGHIKYDPKFQGRVTMTRDTSTSTAYLELSSLRSEDTAVYYCARSGGLPDWWGQGTTVTVSS

SEQ ID NO: 163 - protein, 116 aa, synthetic construct
QVQLVQSGAEVKKPGASVKVSCKASGFDIQDTYMHWVRQRPGQGLEWMGRIDPASGHTKYDPKFQVRATITTDTSTSTAYLELSSLRSEDTAVYYCARSGGLPDVWGQGTTVTVSS

SEQ ID NO: 164 - protein, 116 aa, synthetic construct
QVQLVQSGAEVKKPGASVKVSCKASGFDIQDTYMHWVRQRPGQGLEWIGRIDPASGHTKYDPKFQVRVTITTDTSTSTAYLELSSLRSEDTAVYYCARSGGLPDVWGQGTTVTVSS

SEQ ID NO: 165 - protein, 116 aa, synthetic construct
QVQLVQSGAEVKKPGASVKVSCKASGFDIQDTYMHWVRQRPGQGLEWMGRIDPASGHTKYDPKFQVRVTITTDTSTSTVYLELSSLRSEDTAVYYCARSGGLPDVWGQGTTVTVSS

SEQ ID NO: 166 - protein, 116 aa, synthetic construct
QVQLVQSGAEVKKPGASVKVSCKASGFDIQDTYMHWVRQRPGQGLEWMGRIDPASGHTKYDPKFQVRVTITTDTSTSTAYMELSSLRSEDTAVYYCARSGGLPDVWGQGTTVTVSS

SEQ ID NO: 167 - protein, 116 aa, synthetic construct
QVQLVQSGAEVKKPGASVKVSCKASGFDIQDTYMHWVRQRPGQGLEWMGRIDPASGHTKYDPKFQVRVTMTTDTSTSTAYLELSSLRSEDTAVYYCARSGGLPDVWGQGTTVTVSS

SEQ ID NO: 168 - protein, 116 aa, synthetic construct
QVQLVQSGAEVKKPGASVKVSCKASGFDIQDTYMHWVRQAPGQGLEWMGRIDPASGHIKYDPKFQGRATITTDTSTSTVYMELSSLRSEDTAVYYCARSGGLPDWWGQGTTVTVSS

SEQ ID NO: 169 - protein, 116 aa, synthetic construct
EVQLVQSGAEVKKPGASVKVSCKASGFDIQDTYMHWVRQAPGQGLEWMGRIEPASGHIKYDPKFQGRATITTDTSTSTVYMELSSLRSEDTAVYYCARSGGLPDWWGQGTTVTVSS

SEQ ID NO: 170-200 - skipped (31 entries)

SEQ ID NO: 201 - protein, 106 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 202 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 203 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 204 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWKGNPRTFGGGTKLEIK

SEQ ID NO: 205 - protein, 106 aa, synthetic construct
EIVLTQSPGTLSLSPGERATLSCRASSSVSYMYWYQQKPGQAPRPWIYATSNLASGIPDRFSGSGSGTDFTLTISRLEPEDFAVYYCQQWEGNPRTFGGGTKLEIK

SEQ ID NO: 206 - protein, 106 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWKGNPRTFGGGTKLEIK

SEQ ID NO: 207 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWSRNPRTFGGGTKLEIK

SEQ ID NO: 208 - protein, 106 aa, synthetic construct
EIVLTQSPGTLSASPGERATMSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 209 - protein, 106 aa, synthetic construct
EIVLTQSPGTLSLSPGERATLSCRASSSVSYMYWYQQKPGQAPRLLIYATSNLASGVPDRFSGSGSGTDFTLTISRLEPEDFAVYYCQQWEGNPRTFGGGTKLEIK

SEQ ID NO: 210 - protein, 106 aa, synthetic construct
EIVLTQSPGTLSASPGERATLSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 211 - protein, 106 aa, synthetic construct
EIVLTQSPGTLSASPGERATMSCRASSSVSYMYWYQQKPGQAPRLLIYATSNLASGVPDRFSGSGSGTDYTLTISRVEPEDFAVYYCQQWSGNPRTFGGGTKLEIK

SEQ ID NO: 212 - protein, 106 aa, synthetic construct
EIVLTQSPGTLSASPGERATMSCRASSSVSYMYWYQQKPGQAPRPWIYATSNLASGVPDRFSGSGSGTDFTLTISRVEPEDFAVYYCQQWSGNPRTFGGGTKLEIK

SEQ ID NO: 213 - protein, 106 aa, synthetic construct
EIVLTQSPGTLSASPGERATMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 214 - protein, 106 aa, synthetic construct
EIVLTQSPGTLSASPGERATMSCRASSSVSYMYWYQQKPGQAPRPWIYATSNLASGVPDRFSGSGSGTDFTLTISRLEPEDFAVYYCQQWSGNPRTFGGGTKLEIK

SEQ ID NO: 215 - protein, 106 aa, synthetic construct
EIVLTQSPGTLSASPGERATMSCRASSSVSYMYWYQQKPGQAPRPWIYATSNLASGIPDRFSGSGSGTDYTLTISRVEPEDFAVYYCQQWSGNPRTFGGGTKLEIK

SEQ ID NO: 216 - protein, 106 aa, synthetic construct
EIVLTQSPGTLSLSPGERATLSCRASSSVSYMYWYQQKPGQAPRLLIYATSNLASGIPDRFSGSGSGTDYTLTISRLEPEDFAVYYCQQWEGNPRTFGGGTKLEIK

SEQ ID NO: 217 - protein, 106 aa, synthetic construct
EIVLTQSPGTLSLSPGERATLSCRASSSVSYMYWYQQKPGQAPRLLIYATSNLASGIPDRFSGSGSGTDFTLTISRVEPEDFAVYYCQQWEGNPRTFGGGTKLEIK

SEQ ID NO: 218 - protein, 106 aa, synthetic construct
EIVLTQSPGTLSLSPGERATLSCRASSSVSYMYWYQQKPGQAPRLLIYATSNLASGIPDRFSGSGSGTDYTLTISRVEPEDFAVYYCQQWEGNPRTFGGGTKLEIK

SEQ ID NO: 219 - protein, 106 aa, synthetic construct
EIVLTQSPGTLSASPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 220 - protein, 106 aa, synthetic construct
EIVLTQSPGTMSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 221-300 - skipped (80 entries)

SEQ ID NO: 301 - protein, 118 aa, synthetic construct
  VARIANT 1: note = A, R, N, D, C, Q, E, G, H, I, L, K, M, F, P, S, T, W, Y, or V
  VARIANT 38: note = A, R, N, D, C, Q, E, G, H, I, L, K, M, F, P, S, T, W, Y, or V
  VARIANT 40: note = A, R, N, D, C, Q, E, G, H, I, L, K, M, F, P, S, T, W, Y, or V
  VARIANT 48: note = A, R, N, D, C, Q, E, G, H, I, L, K, M, F, P, S, T, W, Y, or V
  VARIANT 68: note = A, R, N, D, C, Q, E, G, H, I, L, K, M, F, P, S, T, W, Y, or V
  VARIANT 70: note = A, R, N, D, C, Q, E, G, H, I, L, K, M, F, P, S, T, W, Y, or V
  VARIANT 72: note = A, R, N, D, C, Q, E, G, H, I, L, K, M, F, P, S, T, W, Y, or V
  VARIANT 79: note = A, R, N, D, C, Q, E, G, H, I, L, K, M, F, P, S, T, W, Y, or V
  VARIANT 81: note = A, R, N, D, C, Q, E, G, H, I, L, K, M, F, P, S, T, W, Y, or V
  VARIANT 26..35: note = This region may encompass one of the following sequences: GFDIQDTYMH or DTYMH or KYDIN or GFEIQDTYMH or GFDPQDTYMH or GFDVQDTYMH or GFDIGDTYMH or GFDISDTYMH or GFDIVDTYMH or GFDIQDAYMH or GFDIQDSYMH or GFDIQDTFMH or GFDIQDTYIH or GFDLQDTYMH or GFDIQDTYLH or GFDIGDTFIH or GFDIGDTFMH or GFDIGDTYIH or GFDIQDTFIH or GFDIQDTFMH or GFDIQDTYIH or GFDISDTFIH or GFDISDTFMH or GFDISDTYIH or GFDISDTYMH or GFDIVDTFIH or GFDIVDTFMH or GFDIVDTYIH or
  VARIANT 26..35: note = CONT. FROM ABOVE: GFDPGDTFIH or GFDPGDTFMH or GFDPGDTYIH or GFDPGDTYMH or GFDPQDTFIH or GFDPQDTFMH or GFDPQDTYIH or GFDPQDTYMH or GFDPSDTFIH or GFDPSDTFMH or GFDPSDTYIH or GFDPSDTYMH or GFDPVDTFIH or GFDPVDTFMH or GFDPVDTYIH or GFDPVDTYMH or GFDVGDTFIH or GFDVGDTFMH or GFDVGDTYIH or GFDVGDTYMH or GFDVQDTFIH or GFDVQDTFMH or GFDVQDTYIH or GFDVSDTFIH or GFDVSDTFMH or GFDVSDTYIH or GFDVSDTYMH or GFDVVDTFIH or GFDVVDTFMH or GFDVVDTYIH or GFDVVDTYMH or GFEIGDTFIH or GFEIGDTFMH or GFEIGDTYIH or GFEIGDTYMH or GFEIQDTFIH or GFEIQDTFMH or GFEIQDTYIH or GFEIQDTYMH or GFEISDTFIH or GFEISDTFMH or GFEISDTYIH or GFEISDTYMH or GFEIVDTFIH or GFEIVDTFMH or GFEIVDTYIH or GFEIVDTYMH or GFEPGDTFIH or GFEPGDTFMH or GFEPGDTYIH or GFEPGDTYMH or GFEPQDTFIH or GFEPQDTFMH or
  VARIANT 26..35: note = CONT. FROM ABOVE: GFEPQDTYIH or GFEPQDTYMH or GFEPSDTFIH or GFEPSDTFMH or GFEPSDTYIH or GFEPSDTYMH or GFEPVDTFIH or GFEPVDTFMH or GFEPVDTYIH or GFEPVDTYMH or GFEVGDTFIH or GFEVGDTFMH or GFEVGDTYIH or GFEVGDTYMH or GFEVQDTFIH or GFEVQDTFMH or GFEVQDTYIH or GFEVQDTYMH or GFEVSDTFIH or GFEVSDTFMH or GFEVSDTYIH or GFEVSDTYMH or GFEVVDTFIH or GFEVVDTFMH or GFEVVDTYIH or GFEVVDTYMH or GFX1X2X3DX4X5X6H (wherein X1 = D or E, X2 = I, L, P, or V, X3 = G, Q, S, or V, X4 = A, S, or T, X5 = F or Y, and X6 = I, L, or M) or GFX1X2X3DTX4X5H (wherein X1 = D, or E, X2 = I, P, or V, X3 = G, Q, S, or V, X4 = F or Y, and X5 = I or M) or GFTFSSYW or GGSFTGFY or GYX1FX2X3YGIS (wherein X1 = P, S, D, Q, or N, X2 = T or R, and X3 = N, T, Y, or H) or GYDFTYYGIS or SRSYYWG or TSNMGVV or LYGMN or NYGMN or GYTFTSSWMH or GYTFTSYDIN or SYFWS or GYYWN or GFTFSTYG or TYGMS
  VARIANT 50..66: note = This region may encompass one of the following sequences: RIDPASGHTKYDPKFQV or RIEPASGHIKYDPKFQG or RIDPASGHIKYDPKFQG or RIEPASGHIKYDPKFQV or WIFPGDGRTDYNEKFKG or RLDPASGHTK or RIEPASGHTK or RIDPESGHTK or RIDPASGHTK or RIDPAGGHTK or RIDPASAHTK or RIDPASGHIK or RIDPASGHLK or RIDPASGHVK or YDPKFQV or IDPKFQV or LDPKFQV or MDPKFQV or SDPKFQV or TDPKFQV or VDPKFQV or YIPKFQV or YNPKFQV or YRPKFQV or YSPKFQV or YDPKFRV or YDPKFQA or YDPKFQD or YDPKFQE or YDPKFQG or YDPKFQH or YDPKFQK or YDPKFQL or YDPKFQM or YDPKFQN or YDPKFQP or YDPKFQR or YDPKFQS or YDPKFQT or RIEPASGHIKYDPKFQG or RIEPASGHIKYSPKFQG or RIEPASGHVKYSPKFQV or RIEPASGHVKYDPKFQT or RIDPASGHIKYDPKFQK or RIDPASGHVKIDPKFQV or RIDPASGHLKYDPKFQV or
  VARIANT 50..66: note = CONT. FROM ABOVE: RIDPASGHLKYDPKFQR or RIDPASGHLKYDPKFQN or RIEPASGHLKYDPKFQE or PASGH or RIDPASGHTKYDPKFQV or RIDPASGHLKYDPKFQG or RIDPASGHTKYDPKFQG or RIDPASGHSKYDPKFQV or RIDPASGHYKYDPKFQV or RX1X2PX3X4X5HX6KX7X8PKFX9X10 (wherein X1 = I or L, X2 = D or E, X3 = A or E, X4 = G or S, X5 = A or G, X6 = I, L, T, or V, X7 = I, L, M, S, T, V, or Y, X8 = D, I, N, R, or S, X9 = Q or R, and X10 = A, D, E, G, H, K, L, M, N, P, R, S, T, or V) or IKEDGSEK or INHRGNT or WISX1YNGX2X3X4YAX5X6X7QG (wherein X1 = T, P, S, or A, X2 = N, G, V, K, or A, X3 = T or K, X4 = H or N, X5 = Q or R, X6 = K or M, and X7 = L or H) or WISTYNGNTHYARMLQG or SIYYNGRTYYNPSLKS or HILWDDREYSNPALKS or WINTYTGEPTYADDFKG or IHPNSGGT or WLNPNSGXTG (wherein X = N or Y) or YIYYSGNTKYNPSLKS or EINHAGNTNYNPSLKS or ISGTGRTT or WMNTYSGVTTYADDFKG
  VARIANT 99..107: note = This region may encompass one of the following sequences: SGGLPDV or ARSGGLPDV or SGGLPDW or ARSGGLPDW or YGPAMDY or LGGLPDV or SAGLPDV or SGGAPDV or SGGMPDV or SGGLPEV or SGGLPDK or SGGLPDM or SGGLPDQ or SGGLPDR or SGGLPDS or SGGLPDT or SGGLPDH or SGGLPDF or SGGSPDV or ARSGGLPDM or ARSGGLPDK or SGGLPD or ARSGGLPDF or ARSGGLPDL or SGGLPDE or SGGLPDI or SGGLPDK or SGGLPDL or SGGLPDM or SGGLPDQ or SGGLPDT or SGGLPDW or SGGLPDY or SGGMPDE or SGGMPDI or SGGMPDK or SGGMPDL or SGGMPDM or SGGMPDQ or SGGMPDT or SGGMPDV or SGGMPDW or SGGMPDY or X1X2GX3PX4X5 (wherein X1 = L or S, X2 = A or G, X3 = A, L, or M, X4 = D or E, and X5 = K, M, Q, R, S, T, V, or W) or SGGX1PDX2 (wherein X1 = L OR M and X2 = E, I, K, L, M, Q, T, V, W, OR Y)
XVQLVQSGAEVKKPGASVKVSCKASXXXXXXXXXXWVXQXPGQGLEWXGXXXXXXXXXXXXXXXXXRXTXTXDTSTSTXYXELSSLRSEDTAVYYCARXXXXXXXXXWGQGTTVTVSS

SEQ ID NO: 302 - protein, 116 aa, synthetic construct
  VARIANT 1: note = A, R, N, D, C, Q, E, G, H, I, L, K, M, F, P, S, T, W, Y, or V
  VARIANT 38: note = A, R, N, D, C, Q, E, G, H, I, L, K, M, F, P, S, T, W, Y, or V
  VARIANT 40: note = A, R, N, D, C, Q, E, G, H, I, L, K, M, F, P, S, T, W, Y, or V
  VARIANT 48: note = A, R, N, D, C, Q, E, G, H, I, L, K, M, F, P, S, T, W, Y, or V
  VARIANT 68: note = A, R, N, D, C, Q, E, G, H, I, L, K, M, F, P, S, T, W, Y, or V
  VARIANT 70: note = A, R, N, D, C, Q, E, G, H, I, L, K, M, F, P, S, T, W, Y, or V
  VARIANT 72: note = A, R, N, D, C, Q, E, G, H, I, L, K, M, F, P, S, T, W, Y, or V
  VARIANT 79: note = A, R, N, D, C, Q, E, G, H, I, L, K, M, F, P, S, T, W, Y, or V
  VARIANT 81: note = A, R, N, D, C, Q, E, G, H, I, L, K, M, F, P, S, T, W, Y, or V
  VARIANT 26..35: note = This region may encompass one of the following sequences: GFDIQDTYMH or DTYMH or KYDIN or GFEIQDTYMH or GFDPQDTYMH or GFDVQDTYMH or GFDIGDTYMH or GFDISDTYMH or GFDIVDTYMH or GFDIQDAYMH or GFDIQDSYMH or GFDIQDTFMH or GFDIQDTYIH or GFDLQDTYMH or GFDIQDTYLH or GFDIGDTFIH or GFDIGDTFMH or GFDIGDTYIH or GFDIQDTFIH or GFDIQDTFMH or GFDIQDTYIH or GFDISDTFIH or GFDISDTFMH or GFDISDTYIH or GFDISDTYMH or GFDIVDTFIH or GFDIVDTFMH or GFDIVDTYIH or
  VARIANT 26..35: note = CONT. FROM ABOVE: GFDPGDTFIH or GFDPGDTFMH or GFDPGDTYIH or GFDPGDTYMH or GFDPQDTFIH or GFDPQDTFMH or GFDPQDTYIH or GFDPQDTYMH or GFDPSDTFIH or GFDPSDTFMH or GFDPSDTYIH or GFDPSDTYMH or GFDPVDTFIH or GFDPVDTFMH or GFDPVDTYIH or GFDPVDTYMH or GFDVGDTFIH or GFDVGDTFMH or GFDVGDTYIH or GFDVGDTYMH or GFDVQDTFIH or GFDVQDTFMH or GFDVQDTYIH or GFDVSDTFIH or GFDVSDTFMH or GFDVSDTYIH or GFDVSDTYMH or GFDVVDTFIH or GFDVVDTFMH or GFDVVDTYIH or GFDVVDTYMH or GFEIGDTFIH or GFEIGDTFMH or GFEIGDTYIH or GFEIGDTYMH or GFEIQDTFIH or GFEIQDTFMH or GFEIQDTYIH or GFEIQDTYMH or GFEISDTFIH or GFEISDTFMH or GFEISDTYIH or GFEISDTYMH or GFEIVDTFIH or GFEIVDTFMH or GFEIVDTYIH or GFEIVDTYMH or GFEPGDTFIH or GFEPGDTFMH or GFEPGDTYIH or GFEPGDTYMH or GFEPQDTFIH or GFEPQDTFMH or
  VARIANT 26..35: note = CONT. FROM ABOVE: GFEPQDTYIH or GFEPQDTYMH or GFEPSDTFIH or GFEPSDTFMH or GFEPSDTYIH or GFEPSDTYMH or GFEPVDTFIH or GFEPVDTFMH or GFEPVDTYIH or GFEPVDTYMH or GFEVGDTFIH or GFEVGDTFMH or GFEVGDTYIH or GFEVGDTYMH or GFEVQDTFIH or GFEVQDTFMH or GFEVQDTYIH or GFEVQDTYMH or GFEVSDTFIH or GFEVSDTFMH or GFEVSDTYIH or GFEVSDTYMH or GFEVVDTFIH or GFEVVDTFMH or GFEVVDTYIH or GFEVVDTYMH or GFX1X2X3DX4X5X6H (wherein X1 = D or E, X2 = I, L, P, or V, X3 = G, Q, S, or V, X4 = A, S, or T, X5 = F or Y, and X6 = I, L, or M) or GFX1X2X3DTX4X5H (wherein X1 = D, or E, X2 = I, P, or V, X3 = G, Q, S, or V, X4 = F or Y, and X5 = I or M) or GFTFSSYW or GGSFTGFY or GYX1FX2X3YGIS (wherein X1 = P, S, D, Q, or N, X2 = T or R, and X3 = N, T, Y, or H) or GYDFTYYGIS or SRSYYWG or TSNMGVV or LYGMN or NYGMN or GYTFTSSWMH or GYTFTSYDIN or SYFWS or GYYWN or GFTFSTYG or TYGMS
  VARIANT 50..66: note = This region may encompass one of the following sequences: RIDPASGHTKYDPKFQV or RIEPASGHIKYDPKFQG or RIDPASGHIKYDPKFQG or RIEPASGHIKYDPKFQV or WIFPGDGRTDYNEKFKG or RLDPASGHTK or RIEPASGHTK or RIDPESGHTK or RIDPASGHTK or RIDPAGGHTK or RIDPASAHTK or RIDPASGHIK or RIDPASGHLK or RIDPASGHVK or YDPKFQV or IDPKFQV or LDPKFQV or MDPKFQV or SDPKFQV or TDPKFQV or VDPKFQV or YIPKFQV or YNPKFQV or YRPKFQV or YSPKFQV or YDPKFRV or YDPKFQA or YDPKFQD or YDPKFQE or YDPKFQG or YDPKFQH or YDPKFQK or YDPKFQL or YDPKFQM or YDPKFQN or YDPKFQP or YDPKFQR or YDPKFQS or YDPKFQT or RIEPASGHIKYDPKFQG or RIEPASGHIKYSPKFQG or RIEPASGHVKYSPKFQV or RIEPASGHVKYDPKFQT or RIDPASGHIKYDPKFQK or RIDPASGHVKIDPKFQV or RIDPASGHLKYDPKFQV or
  VARIANT 50..66: note = CONT. FROM ABOVE: RIDPASGHLKYDPKFQR or RIDPASGHLKYDPKFQN or RIEPASGHLKYDPKFQE or PASGH or RIDPASGHTKYDPKFQV or RIDPASGHLKYDPKFQG or RIDPASGHTKYDPKFQG or RIDPASGHSKYDPKFQV or RIDPASGHYKYDPKFQV or RX1X2PX3X4X5HX6KX7X8PKFX9X10 (wherein X1 = I or L, X2 = D or E, X3 = A or E, X4 = G or S, X5 = A or G, X6 = I, L, T, or V, X7 = I, L, M, S, T, V, or Y, X8 = D, I, N, R, or S, X9 = Q or R, and X10 = A, D, E, G, H, K, L, M, N, P, R, S, T, or V) or IKEDGSEK or INHRGNT or WISX1YNGX2X3X4YAX5X6X7QG (wherein X1 = T, P, S, or A, X2 = N, G, V, K, or A, X3 = T or K, X4 = H or N, X5 = Q or R, X6 = K or M, and X7 = L or H) or WISTYNGNTHYARMLQG or SIYYNGRTYYNPSLKS or HILWDDREYSNPALKS or WINTYTGEPTYADDFKG or IHPNSGGT or WLNPNSGXTG (wherein X = N or Y) or YIYYSGNTKYNPSLKS or EINHAGNTNYNPSLKS or ISGTGRTT or WMNTYSGVTTYADDFKG
  VARIANT 97..105: note = This region may encompass one of the following sequences: SGGLPDV or ARSGGLPDV or SGGLPDW or ARSGGLPDW or YGPAMDY or LGGLPDV or SAGLPDV or SGGAPDV or SGGMPDV or SGGLPEV or SGGLPDK or SGGLPDM or SGGLPDQ or SGGLPDR or SGGLPDS or SGGLPDT or SGGLPDH or SGGLPDF or SGGSPDV or ARSGGLPDM or ARSGGLPDK or SGGLPD or ARSGGLPDF or ARSGGLPDL or SGGLPDE or SGGLPDI or SGGLPDK or SGGLPDL or SGGLPDM or SGGLPDQ or SGGLPDT or SGGLPDW or SGGLPDY or SGGMPDE or SGGMPDI or SGGMPDK or SGGMPDL or SGGMPDM or SGGMPDQ or SGGMPDT or SGGMPDV or SGGMPDW or SGGMPDY or X1X2GX3PX4X5 (wherein X1 = L or S, X2 = A or G, X3 = A, L, or M, X4 = D or E, and X5 = K, M, Q, R, S, T, V, or W) or SGGX1PDX2 (wherein X1 = L OR M and X2 = E, I, K, L, M, Q, T, V, W, OR Y)
XVQLVQSGAEVKKPGASVKVSCKASXXXXXXXXXXWVXQXPGQGLEWXGXXXXXXXXXXXXXXXXXRXTXTXDTSTSTXYXELSSLRSEDTAVYYCXXXXXXXXXWGQGTTVTVSS

SEQ ID NO: 303 - protein, 113 aa, synthetic construct
  VARIANT 51: note = A, R, N, D, C, Q, E, G, H, I, L, K, M, F, P, S, T, W, Y, or V
  VARIANT 52: note = A, R, N, D, C, Q, E, G, H, I, L, K, M, F, P, S, T, W, Y, or V
  VARIANT 24..39: note = This region may encompass one of the following sequences: RASSSVSYMY or RSSQTIVHSNGDTYLD or GASSSVSYMY or WASSSVSYMY or RASSSVIYMY or RASSSVSFMY or RASSSVSYLY or RASSSVSYMR or GASSSVSYMY or ASSSVSYMY or X1ASSSVX2X3X4X5 (wherein X1 = G, R, or W, X2 = I or S, X3 = F or Y, X4 = L or M, and X5 = R or Y) or QSILYSSNNKNY or QSLVHSDGNTY or RASQSVSSYLA or RASQGISSALA or SASSSVNYMH or KSSQNIVHSDGNTYLE or RSSQSIVHSNGNTYLD or QNINVL or TSSSSDIGAX1X2GVX3 (wherein X1 = G or A, X2 = L, S, or Q, and X3 = H or L) or RASQSINNYLN or RASQSVRSSYLA or QTISSW or RSSQNIVHSDGNTYLE
  VARIANT 55..61: note = This region may encompass one of the following sequences: ATSNLAS or KVSNRFS or AKSNLAS or ATPNLAS or ATENLAS or ATSLLAS or ATSPLAS or ATSNLTS or AX1X2X3LX4S (wherein X1 = K or T, X2 = E, P, or S, X3 = L, N, or P, and X4 = A or T) or WAS or KIS or DASNRAT or DASSLES or STSNLAS or KVSNRFS or KAS or GYYNRPS or AASSLQS or GASSRAT or AAS or KVSNRFS
  VARIANT 94..103: note = This region may encompass one of the following sequences: QQWEGNPRT or QQWKGNPRT or QQWSGNPRT or QQWSRNPRT or FQGSHVPYT or HQWSGNPRT or NQWSGNPRT or SQWSGNPRT or QQSSGNPRT or QQWDGNPRT or QQWHGNPRT or QQWNGNPRT or QQWQGNPRT or QQWVGNPRT or QQWSANPRT or QQWSDNPRT or QQWSQNPRT or QQWSSNPRT or QQWSGNPRS or QQFSGNPRT or QQHSGNPRT or QQISGNPRT or QQPSGNPRT or QQRSGNPRT or QQYSGNPRT or QQWSGHPRT or QQWSGLPRT or QQWSGQPRT or QQWSGSPRT or QQWSGTPRT or QQWSGMPRT or QQWSGFPRT or QQWSGKPRT or QQWSGRPRT or QQWSGDPRT or QQWAGNPRT or QQWYGNPRT or QQWFGNPRT or QQWQGNPRT or GNPRT or SQWSGNPRT or QQWSGNPRS or QQWSGTPRT or QQWSGDPRT or QQWSGFPRT or QQWSGKPRT or QQWSGRPRT or QQWSGSPRT or QQWDADPRT or QQWDAFPRT or QQWDAKPRT or QQWDANPRT or QQWDARPRT or QQWDASPRT or QQWDATPRT or QQWDGDPRT or QQWDGFPRT or QQWDGKPRT or QQWDGNPRT or QQWDGRPRT or QQWDGSPRT or QQWDGTPRT or QQWEADPRT or QQWEAFPRT or QQWEAKPRT or QQWEANPRT or QQWEARPRT or QQWEASPRT or QQWEATPRT or QQWEGDPRT or QQWEGFPRT or QQWEGKPRT or
  VARIANT 94..103: note = CONT. FROM ABOVE: QQWEGRPRT or QQWEGSPRT or QQWEGTPRT or QQWHADPRT or QQWHAFPRT or QQWHAKPRT or QQWHANPRT or QQWHARPRT or QQWHASPRT or QQWHATPRT or QQWHGDPRT or QQWHGFPRT or QQWHGKPRT or QQWHGNPRT or QQWHGRPRT or QQWHGSPRT or QQWHGTPRT or QQWNADPRT or QQWNAFPRT or QQWNAKPRT or QQWNANPRT or QQWNARPRT or QQWNASPRT or QQWNATPRT or QQWNGDPRT or QQWNGFPRT or QQWNGKPRT or QQWNGNPRT or QQWNGRPRT or QQWNGSPRT or QQWNGTPRT or QQWQADPRT or QQWQAFPRT or QQWQAKPRT or QQWQANPRT or QQWQARPRT or QQWQASPRT or QQWQATPRT or QQWQGDPRT or QQWQGFPRT or QQWQGKPRT or QQWQGRPRT or QQWQGSPRT or QQWQGTPRT or QQWSADPRT or QQWSAFPRT or QQWSAKPRT or QQWSANPRT or QQWSARPRT or QQWSASPRT or QQWSATPRT or NQWDAFPRT or NQWDAKPRT or NQWDANPRT or NQWDARPRT or NQWDASPRT or NQWDATPRT or NQWDGDPRT or NQWDGFPRT or NQWDGKPRT or NQWDGNPRT or NQWDGRPRT or NQWDGSPRT or NQWDGTPRT or NQWEADPRT or NQWEAFPRT or
  VARIANT 94..103: note = CONT. FROM ABOVE: NQWEAKPRT or NQWEANPRT or NQWEARPRT or NQWEASPRT or NQWEATPRT or NQWEGDPRT or NQWEGFPRT or NQWEGKPRT or NQWEGNPRT or NQWEGRPRT or NQWEGSPRT or NQWEGTPRT or NQWHADPRT or NQWHAFPRT or NQWHAKPRT or NQWHANPRT or NQWHARPRT or NQWHASPRT or NQWHATPRT or NQWHGDPRT or NQWHGFPRT or NQWHGKPRT or NQWHGNPRT or NQWHGRPRT or NQWHGSPRT or NQWHGTPRT or NQWNADPRT or NQWNAFPRT or NQWNAKPRT or NQWNANPRT or NQWNARPRT or NQWNASPRT or NQWNATPRT or NQWNGDPRT or NQWNGFPRT or NQWNGKPRT or NQWNGNPRT or NQWNGRPRT or NQWNGSPRT or NQWNGTPRT or NQWQADPRT or NQWQAFPRT or NQWQAKPRT or NQWQANPRT or NQWQARPRT or NQWQASPRT or NQWQATPRT or NQWQGDPRT or NQWQGFPRT or NQWQGKPRT or NQWQGNPRT or NQWQGRPRT or NQWQGSPRT or NQWQGTPRT or NQWSADPRT or NQWSAFPRT or NQWSAKPRT or NQWSANPRT or NQWSARPRT or NQWSASPRT or NQWSATPRT or NQWSGDPRT or NQWSGFPRT or NQWSGKPRT or NQWSGNPRT or NQWSGRPRT or
  VARIANT 94..103: note = CONT. FROM ABOVE: NQWSGSPRT or NQWSGTPRT or X1QWX2X3X4PRT (wherein X1 = Q or N, X2 = D, E, H, N, Q, or S, X3 = A or G, and X4 = D, F, K, N, R, S, or T) or X1QX2X3X4X5PRX6 (wherein X1 = H, N, Q, or S, X2 = F, H, I, P, R, S, W, or Y, X3 = D, E, H, N, Q, S, or V, X4 = A, D, G, Q, or S, X5 = D, F, H, K, L, M, N, Q, R, S, or T, and X6 = T or S) or QQYYSTPFT or MQATQFPLT or QQRSNWPWT or QQFNSYPLT or HQWNNYGT or FQGSHVPLT or QQGQSYPYT or QSXDGTLSAL (wherein X = Y, W, or F) or QQSYSTPRT or QQYGSSPT or QQYHRSWT or FQGSHVPLT
EIVLTQSPGTLSLSPGERATLSCXXXXXXXXXXXXXXXXWYQQKPGQAPRXXIYXXXXXXXGIPDRFSGSGSGTDFTLTISRLEPEDFAVYYCXXXXXXXXXXFGGGTKLEIK

SEQ ID NO: 304 - protein, 25 aa, synthetic construct [sequence omitted]

SEQ ID NO: 305 - protein, 14 aa, synthetic construct
WVKQRPGQGLEWMG

SEQ ID NO: 306 - protein, 32 aa, synthetic construct
RVTITRDTSTSTVYLELSSLRSEDTAVYYCAR

SEQ ID NO: 307 - protein, 30 aa, synthetic construct
RVTITRDTSTSTVYLELSSLRSEDTAVYYC

SEQ ID NO: 308 - protein, 11 aa, synthetic construct
WGQGTTVTVSS

SEQ ID NO: 309 - protein, 23 aa, synthetic construct
EIVLTQSPGTLSLSPGERATLSC

SEQ ID NO: 310 - protein, 15 aa, synthetic construct
WYQQKPGQAPRPLIY

SEQ ID NO: 311 - protein, 32 aa, synthetic construct
GIPDRFSGSGSGTDFTLTISRLEPEDFAVYYC

SEQ ID NO: 312 - protein, 10 aa, synthetic construct
FGGGTKLEIK

SEQ ID NO: 313 - protein, 14 aa, synthetic construct
WVRQRPGQGLEWIG

SEQ ID NO: 314 - protein, 32 aa, synthetic construct
RATITTDTSTSTAYLELSSLRSEDTAVYYCAR

SEQ ID NO: 315 - protein, 30 aa, synthetic construct
RATITTDTSTSTAYLELSSLRSEDTAVYYC

SEQ ID NO: 316 - protein, 98 aa, synthetic construct
QVQLVQSGAEVKKPGASVKVSCKASGYTFNSYYMHWVRQAPGQGLEWMGIINPSGGSTSYAQKFQGRVTMTRDTSTSTVYMELSSLRSEDTAVYYCAR

SEQ ID NO: 317 - protein, 96 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 318 - skipped

SEQ ID NO: 319 - protein, 107 aa, synthetic construct [sequence omitted]

SEQ ID NO: 320 - protein, 330 aa, synthetic construct [sequence omitted]

SEQ ID NO: 321 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 330 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 331 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 332 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 339 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 340 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 341 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 343 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 344 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 356 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363-400 - skipped (38 entries)

SEQ ID NO: 401 - protein, 330 aa, synthetic construct; identical to SEQ ID NO: 326

SEQ ID NO: 402 - protein, 330 aa, synthetic construct; identical to SEQ ID NO: 323

SEQ ID NO: 403 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 404 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 405 - protein, 330 aa, synthetic construct; identical to SEQ ID NO: 344

SEQ ID NO: 406 - protein, 330 aa, synthetic construct; identical to SEQ ID NO: 344

SEQ ID NO: 407 - protein, 330 aa, synthetic construct; identical to SEQ ID NO: 344

SEQ ID NO: 408 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEEFGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 409 - protein, 330 aa, synthetic construct; identical to SEQ ID NO: 356

SEQ ID NO: 410 - protein, 330 aa, synthetic construct; identical to SEQ ID NO: 350

SEQ ID NO: 411 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 412 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 413 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 414-500 - skipped (87 entries)

SEQ ID NO: 501 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 502 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 503 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 504 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 505 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 506 - protein, 446 aa, synthetic construct; identical to SEQ ID NO: 505

SEQ ID NO: 507 - protein, 446 aa, synthetic construct; identical to SEQ ID NO: 505

SEQ ID NO: 508 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEEFGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 509 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 510 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 511 - protein, 443 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 512 - protein, 443 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 513 - protein, 443 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 514 - protein, 213 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 515 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 516-600 - skipped (85 entries)

SEQ ID NO: 601 - protein, 5 aa, synthetic construct
DTYMH

SEQ ID NO: 602 - protein, 5 aa, synthetic construct
KYDIN

SEQ ID NO: 603 - protein, 10 aa, synthetic construct
GFEIQDTYMH

SEQ ID NO: 604 - protein, 10 aa, synthetic construct
GFDPQDTYMH

SEQ ID NO: 605 - protein, 10 aa, synthetic construct
GFDVQDTYMH

SEQ ID NO: 606 - protein, 10 aa, synthetic construct
GFDIGDTYMH

SEQ ID NO: 607 - protein, 10 aa, synthetic construct
GFDISDTYMH

SEQ ID NO: 608 - protein, 10 aa, synthetic construct
GFDIVDTYMH

SEQ ID NO: 609 - protein, 10 aa, synthetic construct
GFDIQDAYMH

SEQ ID NO: 610 - protein, 10 aa, synthetic construct
GFDIQDSYMH

SEQ ID NO: 611 - protein, 10 aa, synthetic construct
GFDIQDTFMH

SEQ ID NO: 612 - protein, 10 aa, synthetic construct
GFDIQDTYIH

SEQ ID NO: 613 - protein, 10 aa, synthetic construct
GFDLQDTYMH

SEQ ID NO: 614 - protein, 10 aa, synthetic construct
GFDIQDTYLH

SEQ ID NO: 615 - protein, 10 aa, synthetic construct
GFDIGDTFIH

SEQ ID NO: 616 - protein, 10 aa, synthetic construct
GFDIGDTFMH

SEQ ID NO: 617 - protein, 10 aa, synthetic construct
GFDIGDTYIH

SEQ ID NO: 618 - protein, 10 aa, synthetic construct
GFDIQDTFIH

SEQ ID NO: 619 - protein, 10 aa, synthetic construct
GFDIQDTFMH

SEQ ID NO: 620 - protein, 10 aa, synthetic construct
GFDIQDTYIH

SEQ ID NO: 621 - protein, 10 aa, synthetic construct
GFDISDTFIH

SEQ ID NO: 622 - protein, 10 aa, synthetic construct
GFDISDTFMH

SEQ ID NO: 623 - protein, 10 aa, synthetic construct
GFDISDTYIH

SEQ ID NO: 624 - protein, 10 aa, synthetic construct
GFDISDTYMH

SEQ ID NO: 625 - protein, 10 aa, synthetic construct
GFDIVDTFIH

SEQ ID NO: 626 - protein, 10 aa, synthetic construct
GFDIVDTFMH

SEQ ID NO: 627 - protein, 10 aa, synthetic construct
GFDIVDTYIH

SEQ ID NO: 628 - protein, 10 aa, synthetic construct
GFDPGDTFIH

SEQ ID NO: 629 - protein, 10 aa, synthetic construct
GFDPGDTFMH

SEQ ID NO: 630 - protein, 10 aa, synthetic construct
GFDPGDTYIH

SEQ ID NO: 631 - protein, 10 aa, synthetic construct
GFDPGDTYMH

SEQ ID NO: 632 - protein, 10 aa, synthetic construct
GFDPQDTFIH

SEQ ID NO: 633 - protein, 10 aa, synthetic construct
GFDPQDTFMH

SEQ ID NO: 634 - protein, 10 aa, synthetic construct
GFDPQDTYIH

SEQ ID NO: 635 - protein, 10 aa, synthetic construct
GFDPQDTYMH

SEQ ID NO: 636 - protein, 10 aa, synthetic construct
GFDPSDTFIH

SEQ ID NO: 637 - protein, 10 aa, synthetic construct
GFDPSDTFMH

SEQ ID NO: 638 - protein, 10 aa, synthetic construct
GFDPSDTYIH

SEQ ID NO: 639 - protein, 10 aa, synthetic construct
GFDPSDTYMH

SEQ ID NO: 640 - protein, 10 aa, synthetic construct
GFDPVDTFIH

SEQ ID NO: 641 - protein, 10 aa, synthetic construct
GFDPVDTFMH

SEQ ID NO: 642 - protein, 10 aa, synthetic construct
GFDPVDTYIH

SEQ ID NO: 643 - protein, 10 aa, synthetic construct
GFDPVDTYMH

SEQ ID NO: 644 - protein, 10 aa, synthetic construct
GFDVGDTFIH

SEQ ID NO: 645 - protein, 10 aa, synthetic construct
GFDVGDTFMH

SEQ ID NO: 646 - protein, 10 aa, synthetic construct
GFDVGDTYIH

SEQ ID NO: 647 - protein, 10 aa, synthetic construct
GFDVGDTYMH

SEQ ID NO: 648 - protein, 10 aa, synthetic construct
GFDVQDTFIH

SEQ ID NO: 649 - protein, 10 aa, synthetic construct
GFDVQDTFMH

SEQ ID NO: 650 - protein, 10 aa, synthetic construct
GFDVQDTYIH

SEQ ID NO: 651 - protein, 10 aa, synthetic construct
GFDVSDTFIH

SEQ ID NO: 652 - protein, 10 aa, synthetic construct
GFDVSDTFMH

SEQ ID NO: 653 - protein, 10 aa, synthetic construct
GFDVSDTYIH

SEQ ID NO: 654 - protein, 10 aa, synthetic construct
GFDVSDTYMH

SEQ ID NO: 655 - protein, 10 aa, synthetic construct
GFDVVDTFIH

SEQ ID NO: 656 - protein, 10 aa, synthetic construct
GFDVVDTFMH

SEQ ID NO: 657 - protein, 10 aa, synthetic construct
GFDVVDTYIH

SEQ ID NO: 658 - protein, 10 aa, synthetic construct
GFDVVDTYMH

SEQ ID NO: 659 - protein, 10 aa, synthetic construct
GFEIGDTFIH

SEQ ID NO: 660 - protein, 10 aa, synthetic construct
GFEIGDTFMH

SEQ ID NO: 661 - protein, 10 aa, synthetic construct
GFEIGDTYIH

SEQ ID NO: 662 - protein, 10 aa, synthetic construct
GFEIGDTYMH

SEQ ID NO: 663 - protein, 10 aa, synthetic construct
GFEIQDTFIH

SEQ ID NO: 664 - protein, 10 aa, synthetic construct
GFEIQDTFMH

SEQ ID NO: 665 - protein, 10 aa, synthetic construct
GFEIQDTYIH

SEQ ID NO: 666 - protein, 10 aa, synthetic construct
GFEIQDTYMH

SEQ ID NO: 667 - protein, 10 aa, synthetic construct
GFEISDTFIH

SEQ ID NO: 668 - protein, 10 aa, synthetic construct
GFEISDTFMH

SEQ ID NO: 669 - protein, 10 aa, synthetic construct
GFEISDTYIH

SEQ ID NO: 670 - protein, 10 aa, synthetic construct
GFEISDTYMH

SEQ ID NO: 671 - protein, 10 aa, synthetic construct
GFEIVDTFIH

SEQ ID NO: 672 - protein, 10 aa, synthetic construct
GFEIVDTFMH

SEQ ID NO: 673 - protein, 10 aa, synthetic construct
GFEIVDTYIH

SEQ ID NO: 674 - protein, 10 aa, synthetic construct
GFEIVDTYMH

SEQ ID NO: 675 - protein, 10 aa, synthetic construct
GFEPGDTFIH

SEQ ID NO: 676 - protein, 10 aa, synthetic construct
GFEPGDTFMH

SEQ ID NO: 677 - protein, 10 aa, synthetic construct
GFEPGDTYIH

SEQ ID NO: 678 - protein, 10 aa, synthetic construct
GFEPGDTYMH

SEQ ID NO: 679 - protein, 10 aa, synthetic construct
GFEPQDTFIH

SEQ ID NO: 680 - protein, 10 aa, synthetic construct
GFEPQDTFMH

SEQ ID NO: 681 - protein, 10 aa, synthetic construct
GFEPQDTYIH

SEQ ID NO: 682 - protein, 10 aa, synthetic construct
GFEPQDTYMH

SEQ ID NO: 683 - protein, 10 aa, synthetic construct
GFEPSDTFIH

SEQ ID NO: 684 - protein, 10 aa, synthetic construct
GFEPSDTFMH

SEQ ID NO: 685 - protein, 10 aa, synthetic construct
GFEPSDTYIH

SEQ ID NO: 686 - protein, 10 aa, synthetic construct
GFEPSDTYMH

SEQ ID NO: 687 - protein, 10 aa, synthetic construct
GFEPVDTFIH

SEQ ID NO: 688 - protein, 10 aa, synthetic construct
GFEPVDTFMH

SEQ ID NO: 689 - protein, 10 aa, synthetic construct
GFEPVDTYIH

SEQ ID NO: 690 - protein, 10 aa, synthetic construct
GFEPVDTYMH

SEQ ID NO: 691 - protein, 10 aa, synthetic construct
GFEVGDTFIH

SEQ ID NO: 692 - protein, 10 aa, synthetic construct
GFEVGDTFMH

SEQ ID NO: 693 - protein, 10 aa, synthetic construct
GFEVGDTYIH

SEQ ID NO: 694 - protein, 10 aa, synthetic construct
GFEVGDTYMH

SEQ ID NO: 695 - protein, 10 aa, synthetic construct
GFEVQDTFIH

SEQ ID NO: 696 - protein, 10 aa, synthetic construct
GFEVQDTFMH

SEQ ID NO: 697 - protein, 10 aa, synthetic construct
GFEVQDTYIH

SEQ ID NO: 698 - protein, 10 aa, synthetic construct
GFEVQDTYMH

SEQ ID NO: 699 - protein, 10 aa, synthetic construct
GFEVSDTFIH

SEQ ID NO: 700 - protein, 10 aa, synthetic construct
GFEVSDTFMH

SEQ ID NO: 701 - protein, 10 aa, synthetic construct
GFEVSDTYIH

SEQ ID NO: 702 - protein, 10 aa, synthetic construct
GFEVSDTYMH

SEQ ID NO: 703 - protein, 10 aa, synthetic construct
GFEVVDTFIH

SEQ ID NO: 704 - protein, 10 aa, synthetic construct
GFEVVDTFMH

SEQ ID NO: 705 - protein, 10 aa, synthetic construct
GFEVVDTYIH

SEQ ID NO: 706 - protein, 10 aa, synthetic construct
GFEVVDTYMH

SEQ ID NO: 707 - protein, 10 aa, synthetic construct
  VARIANT 3: note = D or E
  VARIANT 4: note = I, L, P, or V
  VARIANT 5: note = G, Q, S, or V
  VARIANT 7: note = A, S, T
  VARIANT 8: note = F or Y
  VARIANT 9: note = I, L, or M
GFXXXDXXXH

SEQ ID NO: 708 - protein, 10 aa, synthetic construct
  VARIANT 3: note = D or E
  VARIANT 4: note = I, P, or V
  VARIANT 5: note = G, Q, S, or V
  VARIANT 8: note = F or Y
  VARIANT 9: note = I or M
GFXXXDTXXH

SEQ ID NO: 709 - protein, 8 aa, synthetic construct
GFTFSSYW

SEQ ID NO: 710 - protein, 8 aa, synthetic construct
GGSFTGFY

SEQ ID NO: 711 - protein, 10 aa, synthetic construct
  VARIANT 3: note = P, S, D, Q, or N
  VARIANT 5: note = T or R
  VARIANT 6: note = N, T, Y, or H
GYXFXXYGIS

SEQ ID NO: 712 - protein, 10 aa, synthetic construct
GYDFTYYGIS

SEQ ID NO: 713 - protein, 7 aa, synthetic construct
SRSYYWG

SEQ ID NO: 714 - protein, 7 aa, synthetic construct
TSNMGVV

SEQ ID NO: 715 - protein, 5 aa, synthetic construct
LYGMN

SEQ ID NO: 716 - protein, 5 aa, synthetic construct
NYGMN

SEQ ID NO: 717 - protein, 10 aa, synthetic construct
GYTFTSSWMH

SEQ ID NO: 718 - protein, 10 aa, synthetic construct
GYTFTSYDIN

SEQ ID NO: 719 - protein, 5 aa, synthetic construct
SYFWS

SEQ ID NO: 720 - protein, 5 aa, synthetic construct
GYYWN

SEQ ID NO: 721 - protein, 8 aa, synthetic construct
GFTFSTYG

SEQ ID NO: 722 - protein, 5 aa, synthetic construct
TYGMS

SEQ ID NO: 723 - protein, 17 aa, synthetic construct
WIFPGDGRTDYNEKFKG

SEQ ID NO: 724 - protein, 10 aa, synthetic construct
RLDPASGHTK

SEQ ID NO: 725 - protein, 10 aa, synthetic construct
RIEPASGHTK

SEQ ID NO: 726 - protein, 10 aa, synthetic construct
RIDPESGHTK

SEQ ID NO: 727 - protein, 10 aa, synthetic construct
RIDPASGHTK

SEQ ID NO: 728 - protein, 10 aa, synthetic construct
RIDPAGGHTK

SEQ ID NO: 729 - protein, 10 aa, synthetic construct
RIDPASAHTK

SEQ ID NO: 730 - protein, 10 aa, synthetic construct
RIDPASGHIK

SEQ ID NO: 731 - protein, 10 aa, synthetic construct
RIDPASGHLK

SEQ ID NO: 732 - protein, 10 aa, synthetic construct
RIDPASGHVK

SEQ ID NO: 733 - protein, 7 aa, synthetic construct
YDPKFQV

SEQ ID NO: 734 - protein, 7 aa, synthetic construct
IDPKFQV

SEQ ID NO: 735 - protein, 7 aa, synthetic construct
LDPKFQV

SEQ ID NO: 736 - protein, 7 aa, synthetic construct
MDPKFQV

SEQ ID NO: 737 - protein, 7 aa, synthetic construct
SDPKFQV

SEQ ID NO: 738 - protein, 7 aa, synthetic construct
TDPKFQV

SEQ ID NO: 739 - protein, 7 aa, synthetic construct
VDPKFQV

SEQ ID NO: 740 - protein, 7 aa, synthetic construct
YIPKFQV

SEQ ID NO: 741 - protein, 7 aa, synthetic construct
YNPKFQV

SEQ ID NO: 742 - protein, 7 aa, synthetic construct
YRPKFQV

SEQ ID NO: 743 - protein, 7 aa, synthetic construct
YSPKFQV

SEQ ID NO: 744 - protein, 7 aa, synthetic construct
YDPKFRV

SEQ ID NO: 745 - protein, 7 aa, synthetic construct
YDPKFQA

SEQ ID NO: 746 - protein, 7 aa, synthetic construct
YDPKFQD

SEQ ID NO: 747 - protein, 7 aa, synthetic construct
YDPKFQE

SEQ ID NO: 748 - protein, 7 aa, synthetic construct
YDPKFQG

SEQ ID NO: 749 - protein, 7 aa, synthetic construct
YDPKFQH

SEQ ID NO: 750 - protein, 7 aa, synthetic construct
YDPKFQK

SEQ ID NO: 751 - protein, 7 aa, synthetic construct
YDPKFQL

SEQ ID NO: 752 - protein, 7 aa, synthetic construct
YDPKFQM

SEQ ID NO: 753 - protein, 7 aa, synthetic construct
YDPKFQN

SEQ ID NO: 754 - protein, 7 aa, synthetic construct
YDPKFQP

SEQ ID NO: 755 - protein, 7 aa, synthetic construct
YDPKFQR

SEQ ID NO: 756 - protein, 7 aa, synthetic construct
YDPKFQS

SEQ ID NO: 757 - protein, 7 aa, synthetic construct
YDPKFQT

SEQ ID NO: 758 - protein, 17 aa, synthetic construct
RIEPASGHIKYDPKFQG

SEQ ID NO: 759 - protein, 17 aa, synthetic construct
RIEPASGHIKYSPKFQG

SEQ ID NO: 760 - protein, 17 aa, synthetic construct
RIEPASGHVKYSPKFQV

SEQ ID NO: 761 - protein, 17 aa, synthetic construct
RIEPASGHVKYDPKFQT

SEQ ID NO: 762 - protein, 17 aa, synthetic construct
RIDPASGHIKYDPKFQK

SEQ ID NO: 763 - protein, 17 aa, synthetic construct
RIDPASGHVKIDPKFQV

SEQ ID NO: 764 - protein, 17 aa, synthetic construct
RIDPASGHLKYDPKFQV

SEQ ID NO: 765 - protein, 17 aa, synthetic construct
RIDPASGHLKYDPKFQR

SEQ ID NO: 766 - protein, 17 aa, synthetic construct
RIDPASGHLKYDPKFQN

SEQ ID NO: 767 - protein, 17 aa, synthetic construct
RIEPASGHLKYDPKFQE

SEQ ID NO: 768 - protein, 5 aa, synthetic construct
PASGH

SEQ ID NO: 769 - protein, 17 aa, synthetic construct
RIDPASGHTKYDPKFQV

SEQ ID NO: 770 - protein, 17 aa, synthetic construct
RIDPASGHLKYDPKFQG

SEQ ID NO: 771 - protein, 17 aa, synthetic construct
RIDPASGHTKYDPKFQG

SEQ ID NO: 772 - protein, 17 aa, synthetic construct
RIDPASGHSKYDPKFQV

SEQ ID NO: 773 - protein, 17 aa, synthetic construct
RIDPASGHYKYDPKFQV

SEQ ID NO: 774 - protein, 17 aa, synthetic construct
  VARIANT 2: note = I or L
  VARIANT 3: note = D or E
  VARIANT 5: note = A or E
  VARIANT 6: note = G or S
  VARIANT 7: note = A or G
  VARIANT 9: note = I, L, T, or V
  VARIANT 11: note = I, L, M, S, T, V, or Y
  VARIANT 12: note = D, I, N, R, or S
  VARIANT 16: note = Q or R
  VARIANT 17: note = A, D, E, G, H, K, L, M, N, P, R, S, T, or V
RXXPXXXHXKXXPKFXX

SEQ ID NO: 775 - protein, 8 aa, synthetic construct
IKEDGSEK

SEQ ID NO: 776 - protein, 7 aa, synthetic construct
INHRGNT

SEQ ID NO: 777 - protein, 17 aa, synthetic construct
  VARIANT 4: note = T, P, S, or A
  VARIANT 8: note = N, G, V, K, or A
  VARIANT 9: note = T or K
  VARIANT 10: note = H or N
  VARIANT 13: note = Q or R
  VARIANT 14: note = K or M
  VARIANT 15: note = L or H
WISXYNGXXXYAXXXQG

SEQ ID NO: 778 - protein, 17 aa, synthetic construct
WISTYNGNTHYARMLQG

SEQ ID NO: 779 - protein, 16 aa, synthetic construct
SIYYNGRTYYNPSLKS

SEQ ID NO: 780 - protein, 16 aa, synthetic construct
HILWDDREYSNPALKS

SEQ ID NO: 781 - protein, 17 aa, synthetic construct
WINTYTGEPTYADDFKG

SEQ ID NO: 782 - protein, 8 aa, synthetic construct
IHPNSGGT

SEQ ID NO: 783 - protein, 10 aa, synthetic construct
  VARIANT 8: note = N or Y
WLNPNSGXTG

SEQ ID NO: 784 - protein, 16 aa, synthetic construct
YIYYSGNTKYNPSLKS

SEQ ID NO: 785 - protein, 16 aa, synthetic construct
EINHAGNTNYNPSLKS

SEQ ID NO: 786 - protein, 8 aa, synthetic construct
ISGTGRTT

SEQ ID NO: 787 - protein, 17 aa, synthetic construct
WMNTYSGVTTYADDFKG

SEQ ID NO: 788 - protein, 7 aa, synthetic construct
YGPAMDY

SEQ ID NO: 789 - protein, 7 aa, synthetic construct
LGGLPDV

SEQ ID NO: 790 - protein, 7 aa, synthetic construct
SAGLPDV

SEQ ID NO: 791 - protein, 7 aa, synthetic construct
SGGAPDV

SEQ ID NO: 792 - protein, 7 aa, synthetic construct
SGGMPDV

SEQ ID NO: 793 - protein, 7 aa, synthetic construct
SGGLPEV

SEQ ID NO: 794 - protein, 7 aa, synthetic construct
SGGLPDK

SEQ ID NO: 795 - protein, 7 aa, synthetic construct
SGGLPDM

SEQ ID NO: 796 - protein, 7 aa, synthetic construct
SGGLPDQ

SEQ ID NO: 797 - protein, 7 aa, synthetic construct
SGGLPDR

SEQ ID NO: 798 - protein, 7 aa, synthetic construct
SGGLPDS

SEQ ID NO: 799 - protein, 7 aa, synthetic construct
SGGLPDT

SEQ ID NO: 800 - protein, 7 aa, synthetic construct
SGGLPDH

SEQ ID NO: 801 - protein, 7 aa, synthetic construct
SGGLPDF

SEQ ID NO: 802 - protein, 7 aa, synthetic construct
SGGSPDV

SEQ ID NO: 803 - protein, 9 aa, synthetic construct
ARSGGLPDM

SEQ ID NO: 804 - protein, 9 aa, synthetic construct
ARSGGLPDK

SEQ ID NO: 805 - protein, 6 aa, synthetic construct
SGGLPD

SEQ ID NO: 806 - protein, 9 aa, synthetic construct
ARSGGLPDF

SEQ ID NO: 807 - protein, 9 aa, synthetic construct
ARSGGLPDL

SEQ ID NO: 808 - protein, 7 aa, synthetic construct
SGGLPDE

SEQ ID NO: 809 - protein, 7 aa, synthetic construct
SGGLPDI

SEQ ID NO: 810 - protein, 7 aa, synthetic construct
SGGLPDK

SEQ ID NO: 811 - protein, 7 aa, synthetic construct
SGGLPDL

SEQ ID NO: 812 - protein, 7 aa, synthetic construct
SGGLPDM

SEQ ID NO: 813 - protein, 7 aa, synthetic construct
SGGLPDQ

SEQ ID NO: 814 - protein, 7 aa, synthetic construct
SGGLPDT

SEQ ID NO: 815 - protein, 7 aa, synthetic construct
SGGLPDW

SEQ ID NO: 816 - protein, 7 aa, synthetic construct
SGGLPDY

SEQ ID NO: 817 - protein, 7 aa, synthetic construct
SGGMPDE

SEQ ID NO: 818 - protein, 7 aa, synthetic construct
SGGMPDI

SEQ ID NO: 819 - protein, 7 aa, synthetic construct
SGGMPDK

SEQ ID NO: 820 - protein, 7 aa, synthetic construct
SGGMPDL

SEQ ID NO: 821 - protein, 7 aa, synthetic construct
SGGMPDM

SEQ ID NO: 822 - protein, 7 aa, synthetic construct
SGGMPDQ

SEQ ID NO: 823 - protein, 7 aa, synthetic construct
SGGMPDT

SEQ ID NO: 824 - protein, 7 aa, synthetic construct
SGGMPDV

SEQ ID NO: 825 - protein, 7 aa, synthetic construct
SGGMPDW

SEQ ID NO: 826 - protein, 7 aa, synthetic construct
SGGMPDY

SEQ ID NO: 827 - skipped

SEQ ID NO: 828 - protein, 7 aa, synthetic construct
  VARIANT 4: note = L or M
  VARIANT 7: note = E, I, K, L, M, Q, T, V, W, or Y
SGGXPDX

SEQ ID NO: 829 - protein, 15 aa, synthetic construct
AREDYDSYYKYGMDV

SEQ ID NO: 830 - protein, 13 aa, synthetic construct
ASPFYDFWSGSDY

SEQ ID NO: 831 - protein, 15 aa, synthetic construct
  VARIANT 8: note = S or A
  VARIANT 9: note = Y or P
  VARIANT 15: note = V, A, or G
ENYYGSGXXRGGMDX

SEQ ID NO: 832 - protein, 15 aa, synthetic construct
ENYYGSGAYRGGMDV

SEQ ID NO: 833 - protein, 11 aa, synthetic construct
EDYGDYGAFDI

SEQ ID NO: 834 - protein, 14 aa, synthetic construct
MSRNYYGSSYVMDY

SEQ ID NO: 835 - protein, 10 aa, synthetic construct
DTAMDYAMAY

SEQ ID NO: 836 - protein, 13 aa, synthetic construct
DYGKYGDYYAMDY

SEQ ID NO: 837 - protein, 14 aa, synthetic construct
ARGDYYGYVSWFAY

SEQ ID NO: 838 - protein, 10 aa, synthetic construct
EVPETAAFEY

SEQ ID NO: 839 - protein, 10 aa, synthetic construct
ETGSYYGFDY

SEQ ID NO: 840 - protein, 12 aa, synthetic construct
GYCRSTTCYFDY

SEQ ID NO: 841 - protein, 14 aa, synthetic construct
TKERGDYYYGVFDY

SEQ ID NO: 842 - protein, 13 aa, synthetic construct
EGYVFDDYYATDY

SEQ ID NO: 843 - protein, 16 aa, synthetic construct
RSSQTIVHSNGDTYLD

SEQ ID NO: 844 - protein, 10 aa, synthetic construct
GASSSVSYMY

SEQ ID NO: 845 - protein, 10 aa, synthetic construct
WASSSVSYMY

SEQ ID NO: 846 - protein, 10 aa, synthetic construct
RASSSVIYMY

SEQ ID NO: 847 - protein, 10 aa, synthetic construct
RASSSVSFMY

SEQ ID NO: 848 - protein, 10 aa, synthetic construct
RASSSVSYLY

SEQ ID NO: 849 - protein, 10 aa, synthetic construct
RASSSVSYMR

SEQ ID NO: 850 - protein, 10 aa, synthetic construct
GASSSVSYMY

SEQ ID NO: 851 - protein, 9 aa, synthetic construct
ASSSVSYMY

SEQ ID NO: 852 - protein, 10 aa, synthetic construct
  VARIANT 1: note = G, R, or W
  VARIANT 7: note = I or S
  VARIANT 8: note = F or Y
  VARIANT 9: note = L or M
  VARIANT 10: note = R or Y
XASSSVXXXX

SEQ ID NO: 853 - protein, 12 aa, synthetic construct
QSILYSSNNKNY

SEQ ID NO: 854 - protein, 11 aa, synthetic construct
QSLVHSDGNTY

SEQ ID NO: 855 - protein, 11 aa, synthetic construct
RASQSVSSYLA

SEQ ID NO: 856 - protein, 11 aa, synthetic construct
RASQGISSALA

SEQ ID NO: 857 - protein, 10 aa, synthetic construct
SASSSVNYMH

SEQ ID NO: 858 - protein, 16 aa, synthetic construct
KSSQNIVHSDGNTYLE

SEQ ID NO: 859 - protein, 16 aa, synthetic construct
RSSQSIVHSNGNTYLD

SEQ ID NO: 860 - protein, 6 aa, synthetic construct
QNINVL

SEQ ID NO: 861 - protein, 14 aa, synthetic construct
  VARIANT 10: note = G or A
  VARIANT 11: note = L, S, or Q
  VARIANT 14: note = H or L
TSSSSDIGAXXGVX

SEQ ID NO: 862 - protein, 11 aa, synthetic construct
RASQSINNYLN

SEQ ID NO: 863 - protein, 12 aa, synthetic construct
RASQSVRSSYLA

SEQ ID NO: 864 - protein, 6 aa, synthetic construct
QTISSW

SEQ ID NO: 865 - protein, 16 aa, synthetic construct
RSSQNIVHSDGNTYLE

SEQ ID NO: 866 - protein, 7 aa, synthetic construct
KVSNRFS

SEQ ID NO: 867 - protein, 7 aa, synthetic construct
AKSNLAS

SEQ ID NO: 868 - protein, 7 aa, synthetic construct
ATPNLAS

SEQ ID NO: 869 - protein, 7 aa, synthetic construct
ATENLAS

SEQ ID NO: 870 - protein, 7 aa, synthetic construct
ATSLLAS

SEQ ID NO: 871 - protein, 7 aa, synthetic construct
ATSPLAS

SEQ ID NO: 872 - protein, 7 aa, synthetic construct
ATSNLTS

SEQ ID NO: 873-875 - skipped (3 entries)

SEQ ID NO: 876 - protein, 7 aa, synthetic construct
DASNRAT

SEQ ID NO: 877 - protein, 7 aa, synthetic construct
DASSLES

SEQ ID NO: 878 - protein, 7 aa, synthetic construct
STSNLAS

SEQ ID NO: 879 - protein, 7 aa, synthetic construct
KVSNRFS

SEQ ID NO: 880 - skipped

SEQ ID NO: 881 - protein, 7 aa, synthetic construct
GYYNRPS

SEQ ID NO: 882 - protein, 7 aa, synthetic construct
AASSLQS

SEQ ID NO: 883 - protein, 7 aa, synthetic construct
GASSRAT

SEQ ID NO: 884 - skipped

SEQ ID NO: 885 - protein, 7 aa, synthetic construct
KVSNRFS

SEQ ID NO: 886 - protein, 9 aa, synthetic construct
FQGSHVPYT

SEQ ID NO: 887 - protein, 9 aa, synthetic construct
HQWSGNPRT

SEQ ID NO: 888 - protein, 9 aa, synthetic construct
NQWSGNPRT

SEQ ID NO: 889 - protein, 9 aa, synthetic construct
SQWSGNPRT

SEQ ID NO: 890 - protein, 9 aa, synthetic construct
QQSSGNPRT

SEQ ID NO: 891 - protein, 9 aa, synthetic construct
QQWDGNPRT

SEQ ID NO: 892 - protein, 9 aa, synthetic construct
QQWHGNPRT

SEQ ID NO: 893 - protein, 9 aa, synthetic construct
QQWNGNPRT

SEQ ID NO: 894 - protein, 9 aa, synthetic construct
QQWQGNPRT

SEQ ID NO: 895 - protein, 9 aa, synthetic construct
QQWVGNPRT

SEQ ID NO: 896 - protein, 9 aa, synthetic construct
QQWSANPRT

SEQ ID NO: 897 - protein, 9 aa, synthetic construct
QQWSDNPRT

SEQ ID NO: 898 - protein, 9 aa, synthetic construct
QQWSQNPRT

SEQ ID NO: 899 - protein, 9 aa, synthetic construct
QQWSSNPRT

SEQ ID NO: 900 - protein, 9 aa, synthetic construct
QQWSGNPRS

SEQ ID NO: 901 - protein, 9 aa, synthetic construct
QQFSGNPRT

SEQ ID NO: 902 - protein, 9 aa, synthetic construct
QQHSGNPRT

SEQ ID NO: 903 - protein, 9 aa, synthetic construct
QQISGNPRT

SEQ ID NO: 904 - protein, 9 aa, synthetic construct
QQPSGNPRT

SEQ ID NO: 905 - protein, 9 aa, synthetic construct
QQRSGNPRT

SEQ ID NO: 906 - protein, 9 aa, synthetic construct
QQYSGNPRT

SEQ ID NO: 907 - protein, 9 aa, synthetic construct
QQWSGHPRT

SEQ ID NO: 908 - protein, 9 aa, synthetic construct
QQWSGLPRT

SEQ ID NO: 909 - protein, 9 aa, synthetic construct
QQWSGQPRT

SEQ ID NO: 910 - protein, 9 aa, synthetic construct
QQWSGSPRT

SEQ ID NO: 911 - protein, 9 aa, synthetic construct
QQWSGTPRT

SEQ ID NO: 912 - protein, 9 aa, synthetic construct
QQWSGMPRT

SEQ ID NO: 913 - protein, 9 aa, synthetic construct
QQWSGFPRT

SEQ ID NO: 914 - protein, 9 aa, synthetic construct
QQWSGKPRT

SEQ ID NO: 915 - protein, 9 aa, synthetic construct
QQWSGRPRT

SEQ ID NO: 916 - protein, 9 aa, synthetic construct
QQWSGDPRT

SEQ ID NO: 917 - protein, 9 aa, synthetic construct
QQWAGNPRT

SEQ ID NO: 918 - protein, 9 aa, synthetic construct
QQWYGNPRT

SEQ ID NO: 919 - protein, 9 aa, synthetic construct
QQWFGNPRT

SEQ ID NO: 920 - protein, 9 aa, synthetic construct
QQWQGNPRT

SEQ ID NO: 921 - protein, 5 aa, synthetic construct
GNPRT

SEQ ID NO: 922 - protein, 9 aa, synthetic construct
SQWSGNPRT

SEQ ID NO: 923 - protein, 9 aa, synthetic construct
QQWSGNPRS

SEQ ID NO: 924 - protein, 9 aa, synthetic construct
QQWSGTPRT

SEQ ID NO: 925 - protein, 9 aa, synthetic construct
QQWSGDPRT

SEQ ID NO: 926 - protein, 9 aa, synthetic construct
QQWSGFPRT

SEQ ID NO: 927 - protein, 9 aa, synthetic construct
QQWSGKPRT

SEQ ID NO: 928 - protein, 9 aa, synthetic construct
QQWSGRPRT

SEQ ID NO: 929 - protein, 9 aa, synthetic construct
QQWSGSPRT

SEQ ID NO: 930 - protein, 9 aa, synthetic construct
QQWDADPRT

SEQ ID NO: 931 - protein, 9 aa, synthetic construct
QQWDAFPRT

SEQ ID NO: 932 - protein, 9 aa, synthetic construct
QQWDAKPRT

SEQ ID NO: 933 - protein, 9 aa, synthetic construct
QQWDANPRT

SEQ ID NO: 934 - protein, 9 aa, synthetic construct
QQWDARPRT

SEQ ID NO: 935 - protein, 9 aa, synthetic construct
QQWDASPRT

SEQ ID NO: 936 - protein, 9 aa, synthetic construct
QQWDATPRT

SEQ ID NO: 937 - protein, 9 aa, synthetic construct
QQWDGDPRT

SEQ ID NO: 938 - protein, 9 aa, synthetic construct
QQWDGFPRT

SEQ ID NO: 939 - protein, 9 aa, synthetic construct
QQWDGKPRT

SEQ ID NO: 940 - protein, 9 aa, synthetic construct
QQWDGNPRT

SEQ ID NO: 941 - protein, 9 aa, synthetic construct
QQWDGRPRT

SEQ ID NO: 942 - protein, 9 aa, synthetic construct
QQWDGSPRT

SEQ ID NO: 943 - protein, 9 aa, synthetic construct
QQWDGTPRT

SEQ ID NO: 944 - protein, 9 aa, synthetic construct
QQWEADPRT

SEQ ID NO: 945 - protein, 9 aa, synthetic construct
QQWEAFPRT

SEQ ID NO: 946 - protein, 9 aa, synthetic construct
QQWEAKPRT

SEQ ID NO: 947 - protein, 9 aa, synthetic construct
QQWEANPRT

SEQ ID NO: 948 - protein, 9 aa, synthetic construct
QQWEARPRT

SEQ ID NO: 949 - protein, 9 aa, synthetic construct
QQWEASPRT

SEQ ID NO: 950 - protein, 9 aa, synthetic construct
QQWEATPRT

SEQ ID NO: 951 - protein, 9 aa, synthetic construct
QQWEGDPRT

SEQ ID NO: 952 - protein, 9 aa, synthetic construct
QQWEGFPRT

SEQ ID NO: 953 - protein, 9 aa, synthetic construct
QQWEGKPRT

SEQ ID NO: 954 - protein, 9 aa, synthetic construct
QQWEGRPRT

SEQ ID NO: 955 - protein, 9 aa, synthetic construct
QQWEGSPRT

SEQ ID NO: 956 - protein, 9 aa, synthetic construct
QQWEGTPRT

SEQ ID NO: 957 - protein, 9 aa, synthetic construct
QQWHADPRT

SEQ ID NO: 958 - protein, 9 aa, synthetic construct
QQWHAFPRT

SEQ ID NO: 959 - protein, 9 aa, synthetic construct
QQWHAKPRT

SEQ ID NO: 960 - protein, 9 aa, synthetic construct
QQWHANPRT

SEQ ID NO: 961 - protein, 9 aa, synthetic construct
QQWHARPRT

SEQ ID NO: 962 - protein, 9 aa, synthetic construct
QQWHASPRT

SEQ ID NO: 963 - protein, 9 aa, synthetic construct
QQWHATPRT

SEQ ID NO: 964 - protein, 9 aa, synthetic construct
QQWHGDPRT

SEQ ID NO: 965 - protein, 9 aa, synthetic construct
QQWHGFPRT

SEQ ID NO: 966 - protein, 9 aa, synthetic construct
QQWHGKPRT

SEQ ID NO: 967 - protein, 9 aa, synthetic construct
QQWHGNPRT

SEQ ID NO: 968 - protein, 9 aa, synthetic construct
QQWHGRPRT

SEQ ID NO: 969 - protein, 9 aa, synthetic construct
QQWHGSPRT

SEQ ID NO: 970 - protein, 9 aa, synthetic construct
QQWHGTPRT

SEQ ID NO: 971 - protein, 9 aa, synthetic construct
QQWNADPRT

SEQ ID NO: 972 - protein, 9 aa, synthetic construct
QQWNAFPRT

SEQ ID NO: 973 - protein, 9 aa, synthetic construct
QQWNAKPRT

SEQ ID NO: 974 - protein, 9 aa, synthetic construct
QQWNANPRT

SEQ ID NO: 975 - protein, 9 aa, synthetic construct
QQWNARPRT

SEQ ID NO: 976 - protein, 9 aa, synthetic construct
QQWNASPRT

SEQ ID NO: 977 - protein, 9 aa, synthetic construct
QQWNATPRT

SEQ ID NO: 978 - protein, 9 aa, synthetic construct
QQWNGDPRT

SEQ ID NO: 979 - protein, 9 aa, synthetic construct
QQWNGFPRT

SEQ ID NO: 980 - protein, 9 aa, synthetic construct
QQWNGKPRT

SEQ ID NO: 981 - protein, 9 aa, synthetic construct
QQWNGNPRT

SEQ ID NO: 982 - protein, 9 aa, synthetic construct
QQWNGRPRT

SEQ ID NO: 983 - protein, 9 aa, synthetic construct
QQWNGSPRT

SEQ ID NO: 984 - protein, 9 aa, synthetic construct
QQWNGTPRT

SEQ ID NO: 985 - protein, 9 aa, synthetic construct
QQWQADPRT

SEQ ID NO: 986 - protein, 9 aa, synthetic construct
QQWQAFPRT

SEQ ID NO: 987 - protein, 9 aa, synthetic construct
QQWQAKPRT

SEQ ID NO: 988 - protein, 9 aa, synthetic construct
QQWQANPRT

SEQ ID NO: 989 - protein, 9 aa, synthetic construct
QQWQARPRT

SEQ ID NO: 990 - protein, 9 aa, synthetic construct
QQWQASPRT

SEQ ID NO: 991 - protein, 9 aa, synthetic construct
QQWQATPRT

SEQ ID NO: 992 - protein, 9 aa, synthetic construct
QQWQGDPRT

SEQ ID NO: 993 - protein, 9 aa, synthetic construct
QQWQGFPRT

SEQ ID NO: 994 - protein, 9 aa, synthetic construct
QQWQGKPRT

SEQ ID NO: 995 - protein, 9 aa, synthetic construct
QQWQGRPRT

SEQ ID NO: 996 - protein, 9 aa, synthetic construct
QQWQGSPRT

SEQ ID NO: 997 - protein, 9 aa, synthetic construct
QQWQGTPRT

SEQ ID NO: 998 - protein, 9 aa, synthetic construct
QQWSADPRT

SEQ ID NO: 999 - protein, 9 aa, synthetic construct
QQWSAFPRT

SEQ ID NO: 1000 - protein, 9 aa, synthetic construct
QQWSAKPRT

SEQ ID NO: 1001 - protein, 9 aa, synthetic construct
QQWSANPRT

SEQ ID NO: 1002 - protein, 9 aa, synthetic construct
QQWSARPRT

SEQ ID NO: 1003 - protein, 9 aa, synthetic construct
QQWSASPRT

SEQ ID NO: 1004 - protein, 9 aa, synthetic construct
QQWSATPRT

SEQ ID NO: 1005 - protein, 9 aa, synthetic construct
NQWDAFPRT

SEQ ID NO: 1006 - protein, 9 aa, synthetic construct
NQWDAKPRT

SEQ ID NO: 1007 - protein, 9 aa, synthetic construct
NQWDANPRT

SEQ ID NO: 1008 - protein, 9 aa, synthetic construct
NQWDARPRT

SEQ ID NO: 1009 - protein, 9 aa, synthetic construct
NQWDASPRT

SEQ ID NO: 1010 - protein, 9 aa, synthetic construct
NQWDATPRT

SEQ ID NO: 1011 - protein, 9 aa, synthetic construct
NQWDGDPRT

SEQ ID NO: 1012 - protein, 9 aa, synthetic construct
NQWDGFPRT

SEQ ID NO: 1013 - protein, 9 aa, synthetic construct
NQWDGKPRT

SEQ ID NO: 1014 - protein, 9 aa, synthetic construct
NQWDGNPRT

SEQ ID NO: 1015 - protein, 9 aa, synthetic construct
NQWDGRPRT

SEQ ID NO: 1016 - protein, 9 aa, synthetic construct
NQWDGSPRT

SEQ ID NO: 1017 - protein, 9 aa, synthetic construct
NQWDGTPRT

SEQ ID NO: 1018 - protein, 9 aa, synthetic construct
NQWEADPRT

SEQ ID NO: 1019 - protein, 9 aa, synthetic construct
NQWEAFPRT

SEQ ID NO: 1020 - protein, 9 aa, synthetic construct
NQWEAKPRT

SEQ ID NO: 1021 - protein, 9 aa, synthetic construct
NQWEANPRT

SEQ ID NO: 1022 - protein, 9 aa, synthetic construct
NQWEARPRT

SEQ ID NO: 1023 - protein, 9 aa, synthetic construct
NQWEASPRT

SEQ ID NO: 1024 - protein, 9 aa, synthetic construct
NQWEATPRT

SEQ ID NO: 1025 - protein, 9 aa, synthetic construct
NQWEGDPRT

SEQ ID NO: 1026 - protein, 9 aa, synthetic construct
NQWEGFPRT

SEQ ID NO: 1027 - protein, 9 aa, synthetic construct
NQWEGKPRT

SEQ ID NO: 1028 - protein, 9 aa, synthetic construct
NQWEGNPRT

SEQ ID NO: 1029 - protein, 9 aa, synthetic construct
NQWEGRPRT

SEQ ID NO: 1030 - protein, 9 aa, synthetic construct
NQWEGSPRT

SEQ ID NO: 1031 - protein, 9 aa, synthetic construct
NQWEGTPRT

SEQ ID NO: 1032 - protein, 9 aa, synthetic construct
NQWHADPRT

SEQ ID NO: 1033 - protein, 9 aa, synthetic construct
NQWHAFPRT

SEQ ID NO: 1034 - protein, 9 aa, synthetic construct
NQWHAKPRT

SEQ ID NO: 1035 - protein, 9 aa, synthetic construct
NQWHANPRT

SEQ ID NO: 1036 - protein, 9 aa, synthetic construct
NQWHARPRT

SEQ ID NO: 1037 - protein, 9 aa, synthetic construct
NQWHASPRT

SEQ ID NO: 1038 - protein, 9 aa, synthetic construct
NQWHATPRT

SEQ ID NO: 1039 - protein, 9 aa, synthetic construct
NQWHGDPRT

SEQ ID NO: 1040 - protein, 9 aa, synthetic construct
NQWHGFPRT

SEQ ID NO: 1041 - protein, 9 aa, synthetic construct
NQWHGKPRT

SEQ ID NO: 1042 - protein, 9 aa, synthetic construct
NQWHGNPRT

SEQ ID NO: 1043 - protein, 9 aa, synthetic construct
NQWHGRPRT

SEQ ID NO: 1044 - protein, 9 aa, synthetic construct
NQWHGSPRT

SEQ ID NO: 1045 - protein, 9 aa, synthetic construct
NQWHGTPRT

SEQ ID NO: 1046 - protein, 9 aa, synthetic construct
NQWNADPRT

SEQ ID NO: 1047 - protein, 9 aa, synthetic construct
NQWNAFPRT

SEQ ID NO: 1048 - protein, 9 aa, synthetic construct
NQWNAKPRT

SEQ ID NO: 1049 - protein, 9 aa, synthetic construct
NQWNANPRT

SEQ ID NO: 1050 - protein, 9 aa, synthetic construct
NQWNARPRT

SEQ ID NO: 1051 - protein, 9 aa, synthetic construct
NQWNASPRT

SEQ ID NO: 1052 - protein, 9 aa, synthetic construct
NQWNATPRT

SEQ ID NO: 1053 - protein, 9 aa, synthetic construct
NQWNGDPRT

SEQ ID NO: 1054 - protein, 9 aa, synthetic construct
NQWNGFPRT

SEQ ID NO: 1055 - protein, 9 aa, synthetic construct
NQWNGKPRT

SEQ ID NO: 1056 - protein, 9 aa, synthetic construct
NQWNGNPRT

SEQ ID NO: 1057 - protein, 9 aa, synthetic construct
NQWNGRPRT

SEQ ID NO: 1058 - protein, 9 aa, synthetic construct
NQWNGSPRT

SEQ ID NO: 1059 - protein, 9 aa, synthetic construct
NQWNGTPRT

SEQ ID NO: 1060 - protein, 9 aa, synthetic construct
NQWQADPRT

SEQ ID NO: 1061 - protein, 9 aa, synthetic construct
NQWQAFPRT

SEQ ID NO: 1062 - protein, 9 aa, synthetic construct
NQWQAKPRT

SEQ ID NO: 1063 - protein, 9 aa, synthetic construct
NQWQANPRT

SEQ ID NO: 1064 - protein, 9 aa, synthetic construct
NQWQARPRT

SEQ ID NO: 1065 - protein, 9 aa, synthetic construct
NQWQASPRT

SEQ ID NO: 1066 - protein, 9 aa, synthetic construct
NQWQATPRT

SEQ ID NO: 1067 - protein, 9 aa, synthetic construct
NQWQGDPRT

SEQ ID NO: 1068 - protein, 9 aa, synthetic construct
NQWQGFPRT

SEQ ID NO: 1069 - protein, 9 aa, synthetic construct
NQWQGKPRT

SEQ ID NO: 1070 - protein, 9 aa, synthetic construct
NQWQGNPRT

SEQ ID NO: 1071 - protein, 9 aa, synthetic construct
NQWQGRPRT

SEQ ID NO: 1072 - protein, 9 aa, synthetic construct
NQWQGSPRT

SEQ ID NO: 1073 - protein, 9 aa, synthetic construct
NQWQGTPRT

SEQ ID NO: 1074 - protein, 9 aa, synthetic construct
NQWSADPRT

SEQ ID NO: 1075 - protein, 9 aa, synthetic construct
NQWSAFPRT

SEQ ID NO: 1076 - protein, 9 aa, synthetic construct
NQWSAKPRT

SEQ ID NO: 1077 - protein, 9 aa, synthetic construct
NQWSANPRT

SEQ ID NO: 1078 - protein, 9 aa, synthetic construct
NQWSARPRT

SEQ ID NO: 1079 - protein, 9 aa, synthetic construct
NQWSASPRT

SEQ ID NO: 1080 - protein, 9 aa, synthetic construct
NQWSATPRT

SEQ ID NO: 1081 - protein, 9 aa, synthetic construct
NQWSGDPRT

SEQ ID NO: 1082 - protein, 9 aa, synthetic construct
NQWSGFPRT

SEQ ID NO: 1083 - protein, 9 aa, synthetic construct
NQWSGKPRT

SEQ ID NO: 1084 - protein, 9 aa, synthetic construct
NQWSGNPRT

SEQ ID NO: 1085 - protein, 9 aa, synthetic construct
NQWSGRPRT

SEQ ID NO: 1086 - protein, 9 aa, synthetic construct
NQWSGSPRT

SEQ ID NO: 1087 - protein, 9 aa, synthetic construct
NQWSGTPRT

SEQ ID NO: 1088 - protein, 9 aa, synthetic construct
  VARIANT 1: note = Q or N
  VARIANT 4: note = D, E, H, N, Q, or S
  VARIANT 5: note = A or G
  VARIANT 6: note = D, F, K, N, R, S, or T
XQWXXXPRT

SEQ ID NO: 1089 - skipped

SEQ ID NO: 1090 - protein, 9 aa, synthetic construct
QQYYSTPFT

SEQ ID NO: 1091 - protein, 9 aa, synthetic construct
MQATQFPLT

SEQ ID NO: 1092 - protein, 9 aa, synthetic construct
QQRSNWPWT

SEQ ID NO: 1093 - protein, 9 aa, synthetic construct
QQFNSYPLT

SEQ ID NO: 1094 - protein, 8 aa, synthetic construct
HQWNNYGT

SEQ ID NO: 1095 - protein, 9 aa, synthetic construct
FQGSHVPLT

SEQ ID NO: 1096 - protein, 9 aa, synthetic construct
QQGQSYPYT

SEQ ID NO: 1097 - protein, 10 aa, synthetic construct
  VARIANT 3: note = Y, W, or F
QSXDGTLSAL

SEQ ID NO: 1098 - protein, 9 aa, synthetic construct
QQSYSTPRT

SEQ ID NO: 1099 - protein, 8 aa, synthetic construct
QQYGSSPT

SEQ ID NO: 1100 - protein, 8 aa, synthetic construct
QQYHRSWT

SEQ ID NO: 1101 - protein, 9 aa, synthetic construct
FQGSHVPLT

SEQ ID NO: 1102-1199 - skipped (98 entries)

SEQ ID NO: 1200 - protein, 116 aa, synthetic construct
EVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 1201 - protein, 116 aa, synthetic construct
QVHLQQSGPELVKPGASVKLSCKASGYTFTKYDINWVRQRPEQGLEWIGWIFPGDGRTDYNEKFKGKATLTTDKSSSTAYMEVSRLTSEDSAVYFCARYGPAMDYWGQGTSVTVAS

SEQ ID NO: 1202 - protein, 116 aa, synthetic construct
QVQLVQSGAEVKKPGASVKVSCKASGFDICDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 1203 - protein, 116 aa, synthetic construct
QVQLVQSGAEVKKPGASVKLSCKASGFDICDTYMHWVRQRPGQGLEWIGRIDPASGHTKYDPKFQVRATMTTDTSTSTVYLELSSLRSEDTAVYYCARSGGLPDVWGQGTTVTVSS

SEQ ID NO: 1204 - protein, 116 aa, synthetic construct
QVQLVQSGAEVVKPGASVKLSCKASGFDICDTYMHWVRQRPGQGLEWMGRIDPASGHTKYDPKFQVRVTMTTDTSTSTVYLELSSLRSEDTAVYYCARSGGLPDVWGQGTTVTVSS

SEQ ID NO: 1205 - protein, 116 aa, synthetic construct
QVQLVQSGAEVKKPGASVKVSCKASGFDICDTYMHWVKQRPGQGLEWIGRIDPASGHTKYDPKFQVRATITRDTSTSTAYLELSSLRSEDTAVYYCSRSGGLPDVWGQGTTVTVSS

SEQ ID NO: 1206 - protein, 116 aa, synthetic construct
QVQLVQSGAEVKKPGASVKVSCKASGFDICDTYMHWVKQAPGQGLEWMGRIDPASGHTKYDPKFQVRATMTTDTSTSTAYLELSSLRSEDTAVYYCSRSGGLPDVWGQGTTVTVSS

SEQ ID NO: 1207 - protein, 116 aa, synthetic construct
QVQLVQSGAEVKKPGASVKLSCKASGFDIQDTYMHWVRQAPGQGLEWMGRIDPASGHTKYDPKFQVRVTMTTDTSTSTVYMELSSLRSEDTAVYYCSRSGGLPDVWGQGTTVTVSS

SEQ ID NO: 1208 - protein, 116 aa, synthetic construct
QVQLVQSGAEVKKPGASVKLSCKASGFDIQDTYMHWVRQAPGQGLEWMGRIDPASGHTKYDPKFQVRVTMTRDTSTSTVYMELSSLRSEDTAVYYCSRSGGLPDVWGQGTTVTVSS

SEQ ID NO: 1209 - protein, 116 aa, synthetic construct
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 1210 - protein, 116 aa, synthetic construct
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDKWGQGTTVTVSS

SEQ ID NO: 1211 - protein, 116 aa, synthetic construct
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDMWGQGTTVTVSS

SEQ ID NO: 1212 - protein, 116 aa, synthetic construct
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDQWGQGTTVTVSS

SEQ ID NO: 1213 - protein, 116 aa, synthetic construct
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDWWGQGTTVTVSS

SEQ ID NO: 1214 - protein, 116 aa, synthetic construct
  VARIANT 28: note = D or E
  VARIANT 29: note = I, P, or V
  VARIANT 30: note = G, Q, S, or V
  VARIANT 33: note = F or Y
  VARIANT 34: note = I or M
  VARIANT 102: note = L or M
  VARIANT 105: note = E, I, K, L, M, Q, T, V, W, or Y
QVQLVQSGAEVKKPGASVKVSCKASGFXXXDTXXHWVRQAPGQGLEWMGRIDPASGHTKYDPKFQVRVTMTRDTSTSTVYMELSSLRSEDTAVYYCARSGGXPDXWGQGTTVTVSS

SEQ ID NO: 1215 - protein, 116 aa, synthetic construct
  VARIANT 28: note = D or E
  VARIANT 29: note = I, P, or V
  VARIANT 30: note = G, Q, S, or V
  VARIANT 33: note = F or Y
  VARIANT 34: note = I or M
  VARIANT 102: note = L or M
  VARIANT 105: note = E, I, K, L, M, Q, T, V, W, or Y
QVQLVQSGAEVKKPGASVKVSCKASGFXXXDTXXHWVRQAPGQGLEWMGRIDPASGHTKYDPKFQVRVTMTRDTSTSTVYMELSSLRSEDTAVYYCSRSGGXPDXWGQGTTVTVSS

SEQ ID NO: 1216 - protein, 116 aa, synthetic construct
QVQLVQSGAEVKKPGASVKVSCKASGFDIQDTYMHWVRQAPGQGLEWMGRIEPASGHIKYSPKFQGRVTMTRDTSTSTVYMELSSLRSEDTAVYYCARSGGLPDWWGQGTTVTVSS

SEQ ID NO: 1217 - protein, 116 aa, synthetic construct
QVQLVQSGAEVKKPGASVKVSCKASGFDIQDTYMHWVRQAPGQGLEWMGRIEPASGHVKYSPKFQVRVTMTRDTSTSTVYMELSSLRSEDTAVYYCARSGGLPDWWGQGTTVTVSS

SEQ ID NO: 1218 - protein, 116 aa, synthetic construct
QVQLVQSGAEVKKPGASVKVSCKASGFDIQDTYMHWVRQAPGQGLEWMGRIEPASGHVKYDPKFQTRVTMTRDTSTSTVYMELSSLRSEDTAVYYCARSGGLPDWWGQGTTVTVSS

SEQ ID NO: 1219 - protein, 116 aa, synthetic construct
QVQLVQSGAEVKKPGASVKVSCKASGFDIQDTYMHWVRQAPGQGLEWMGRIDPASGHIKYDPKFQKRVTMTRDTSTSTVYMELSSLRSEDTAVYYCARSGGLPDMWGQGTTVTVSS

SEQ ID NO: 1220 - protein, 116 aa, synthetic construct
QVQLVQSGAEVKKPGASVKVSCKASGFDIQDTYMHWVRQAPGQGLEWMGRIDPASGHVKIDPKFQVRVTMTRDTSTSTVYMELSSLRSEDTAVYYCARSGGLPDMWGQGTTVTVSS

SEQ ID NO: 1221 - protein, 116 aa, synthetic construct
QVQLVQSGAEVKKPGASVKVSCKASGFDIQDTYMHWVRQAPGQGLEWMGRIDPASGHLKYDPKFQVRVTMTRDTSTSTVYMELSSLRSEDTAVYYCARSGGLPDMWGQGTTVTVSS

SEQ ID NO: 1222 - protein, 116 aa, synthetic construct
QVQLVQSGAEVKKPGASVKVSCKASGFDIQDTYMHWVRQAPGQGLEWMGRIDPASGHLKYDPKFQRRVTMTRDTSTSTVYMELSSLRSEDTAVYYCARSGGLPDMWGQGTTVTVSS

SEQ ID NO: 1223 - protein, 116 aa, synthetic construct
QVQLVQSGAEVKKPGASVKVSCKASGFDIQDTYMHWVRQAPGQGLEWMGRIDPASGHLKYDPKFQNRVTMTRDTSTSTVYMELSSLRSEDTAVYYCARSGGLPDKWGQGTTVTVSS

SEQ ID NO: 1224 - protein, 116 aa, synthetic construct
QVQLVQSGAEVKKPGASVKVSCKASGFDIQDTYMHWVRQAPGQGLEWMGRIEPASGHLKYDPKFQERVTMTRDTSTSTVYMELSSLRSEDTAVYYCARSGGLPDKWGQGTTVTVSS

SEQ ID NO: 1225 - protein, 116 aa, synthetic construct
QVQLVQSGAEVKKPGASVKVSCKASGFDIQDTYMHWVRQAPGQGLEWMGRIDPASGHLKYDPKFQGRVTITRDTSASTAYMELSSLRSEDTAVYYCARSGGLPDMWGQGTTVTVSS

SEQ ID NO: 1226 - protein, 116 aa, synthetic construct
QVQLVQSGAEVKKPGASVKVSCKASGFDIQDTYMHWVRQAPGQGLEWMGRIDPASGHLKYDPKFQGRVTITRDTSASTVYMELSSLRSEDTAVYYCARSGGLPDMWGQGTTVTVSS

SEQ ID NO: 1227 - protein, 116 aa, synthetic construct
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDVWGQGTTVTVSS

SEQ ID NO: 1228 - protein, 116 aa, synthetic construct
QVQLVQSGAEVKKPGASVKVSCKASGFDIQDTYMHWVRQAPGQGLEWMGRIDPASGHTKYDPKFQVRATITTDTSASTAYLQLSSLRSEDTAVYYCARSGGLPDFWGQGTTVTVSS

SEQ ID NO: 1229 - protein, 116 aa, synthetic construct
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDFWGQGTTVTVSS

SEQ ID NO: 1230 - protein, 116 aa, synthetic construct
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDLWGQGTTVTVSS

SEQ ID NO: 1231 - protein, 116 aa, synthetic construct
QVQLVQSGAEVKKPGASVKVSCKASGFDIQDTYMHWVRQAPGQGLEWMGRIDPASGHSKYDPKFQVRATITTDTSASTAYLQLSSLRSEDTAVYYCARSGGLPDFWGQGTTVTVSS

SEQ ID NO: 1232 - protein, 116 aa, synthetic construct
QVQLVQSGAEVKKPGASVKVSCKASGFDIQDTYMHWVRQAPGQGLEWMGRIDPASGHYKYDPKFQVRATITTDTSASTAYLQLSSLRSEDTAVYYCARSGGLPDFWGQGTTVTVSS

SEQ ID NO: 1233 - protein, 122 aa, synthetic construct
EVQLVESGGGLVQPGGSLRLSCAVSGFTFSSYWMSWVRQAPGKGLEWVANIKEDGSEKNYVDSVKGRFTLSSDNAKNSLYLQMNSLRAEDTAVYYCAREDYDSYYKYGMDVWGQGTAVIVSS

SEQ ID NO: 1234 - protein, 119 aa, synthetic construct
QVQLQQWGAGLLKPSETLSLTCAVYGGSFTGFYWSWIRQPPGKGLEWIGEINHRGNTNYNPSLKSRVTMSVDTSKNQFSLNMISVTAADTAMYFCASPFYDFWSGSDYWGQGTLVTVSS

SEQ ID NO: 1235 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 1236 - protein, 453 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1237 - protein, 121 aa, synthetic construct
QLQLQESGPGLVKPSETLSLTCTVSGGSISSRSYYWGWIRQPPGKGLEWIGSIYYNGRTYYNPSLKSRVTISVDTSKNQFSLKLSSVTAADTAVYYCAREDYGDYGAFDIWGQGTMVTVSS

SEQ ID NO: 1238 - protein, 124 aa, synthetic construct
QVTLKESGPALVKPTQTLTLTCTFSGFSLSTSNMGVVWIRQPPGKALEWLAHILWDDREYSNPALKSRLTISKDTSKNQVVLTMTNMDPVDTATYYCARMSRNYYGSSYVMDYWGQGTLVTVSS

SEQ ID NO: 1239 - protein, 119 aa, synthetic construct
QVQLVQSGSELKKPGASVKVSCKASGYTFTLYGMNWVRQAPGQGLEWMGWINTYTGEPTYADDFKGRFVFSLDTSVSTAYLQISSLKAEDTAVYYCARDTAMDYAMAYWGQGTLVTVSS

SEQ ID NO: 1240 - protein, 119 aa, synthetic construct
QVQLVQSGSELKKPGASVKVSCKASGYTFTLYGMNWVKQAPGKGLKWMGWINTYTGEPTYADDFKGRFVFSLDTSVSTAYLQISSLKAEDTAVYFCARDTAMDYAMAYWGQGTLVTVSS

SEQ ID NO: 1241 - protein, 122 aa, synthetic construct
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARDYGKYGDYYAMDYWGQGTLVTVSS

SEQ ID NO: 1242 - protein, 122 aa, synthetic construct
QVQLVQSGSELKKPGASVKVSCKASGYTFTNYGMNWVRQAPGKGLKWMGWINTYTGEPTYADDFKGRFVFSLDTSVSTAYLQISSLKAEDTAVYFCARDYGKYGDYYAMDYWGQGTLVTVSS

SEQ ID NO: 1243 - protein, 119 aa, synthetic construct
QVQLQQPGSVLVRPGASVKVSCKASGYTFTSSWMHWAKQRPGQGLEWIGEIHPNSGGTNYNEKFKGKATVDTSSSTAYVDLSSLTSEDSAVYYCARGDYYGYVSWFAYWGQGTLVTVSS

SEQ ID NO: 1244 - protein, 121 aa, synthetic construct
QVQLVQSGAEVKKPGASVKVSCKASGYTFTSSWMHWARQAPGQGLEWIGEIHPNSGGTNYAQKFQGRATLTVDTSSSTAYMELSRLRSDDTAVYYCARGDYYGYVSWFAYWGQGTLVTVSS

SEQ ID NO: 1245 - protein, 121 aa, synthetic construct
QVQLVQSGAEVKKPGASVKVSCKASGYTFTSSWMHWARQAPGQGLEWIGEIHPNSGGTNYAQKFQGRATMTVDTSISTAYMELSRLRSDDTAVYYCARGDYYGYVSWFAYWGQGTLVTVSS

SEQ ID NO: 1246 - protein, 121 aa, synthetic construct
QVQLVQSGAEVKKPGASVKVSCKASGYTFTSSWMHWARQAPGQGLEWIGEIHPNSGGTNYAQKFQGRVTMTVDTSISTAYMELSRLRSDDTAVYYCARGDYYGYVSWFAYWGQGTLVTVSS

SEQ ID NO: 1247 - protein, 121 aa, synthetic construct
QVQLVQSGAEVKKPGASVKVSCKASGYTFTSSWMHWARQAPGQGLEWMGEIHPNSGGTNYAQKFQGRVTMTVDTSISTAYMELSRLRSDDTAVYYCARGDYYGYVSWFAYWGQGTLVTVSS

SEQ ID NO: 1248 - protein, 119 aa, synthetic construct
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 1249 - protein, 119 aa, synthetic construct; identical to SEQ ID NO: 1248

SEQ ID NO: 1250 - protein, 119 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 1251 - protein, 119 aa, synthetic construct; identical to SEQ ID NO: 1248

SEQ ID NO: 1252 - protein, 119 aa, synthetic construct; identical to SEQ ID NO: 1248

SEQ ID NO: 1253 - protein, 119 aa, synthetic construct; identical to SEQ ID NO: 1248

SEQ ID NO: 1254 - protein, 119 aa, synthetic construct; identical to SEQ ID NO: 1248

SEQ ID NO: 1255 - protein, 119 aa, synthetic construct; identical to SEQ ID NO: 1250

SEQ ID NO: 1256 - protein, 119 aa, synthetic construct; identical to SEQ ID NO: 1250

SEQ ID NO: 1257 - protein, 119 aa, synthetic construct; identical to SEQ ID NO: 1250

SEQ ID NO: 1258 - protein, 119 aa, synthetic construct; identical to SEQ ID NO: 1250

SEQ ID NO: 1259 - protein, 119 aa, synthetic construct; identical to SEQ ID NO: 1250

SEQ ID NO: 1260 - protein, 118 aa, synthetic construct
QVQLQESGPGLVKPSETLSLTCTVSGGSISSYFWSWIRQPPGKGLEWIGYIYYSGNTKYNPSLKSRVTISIDTSKNQFSLKLSSVTAADTAVYYCARETGSYYGFDYWGQGTLVTVSS

SEQ ID NO: 1261 - protein, 120 aa, synthetic construct
QVQLQQWGAGLLKPSETLSLTCAVHGGSFSGYYWNWIRQPPGKGLEWIGEINHAGNTNYNPSLKSRVTISLDTSKNQFSLTLTSVTAADTAVYYCARGYCRSTTCYFDYWGQGTLVTVSS

SEQ ID NO: 1262 - protein, 121 aa, synthetic construct
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 1263 - protein, 122 aa, synthetic construct
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 1264 - protein, 106 aa, synthetic construct
QIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 1265 - protein, 131 aa, synthetic construct
MKLPVRLLVLMFWIPASSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 1266 - protein, 106 aa, synthetic construct
EIVLTQSPGTLSLSPGERATMSCRASSSVSYMYWYQQKPGQAPRPWIYATSNLASGIPDRFSGSGSGTDFTLTISRVEPEDFAVYYCQQWSGNPRTFGGGTKLEIK

SEQ ID NO: 1267 - protein, 106 aa, synthetic construct
EIVLTQSPGTLSLSPGERVTMSCRASSSVSYMYWYQQKPGQAPRPWIYATSNLASGVPDRFSGSGSGTDFTLTISRVEPEDFAVYYCQQWSGNPRTFGGGTKVEIK

SEQ ID NO: 1268 - protein, 106 aa, synthetic construct
EIVLTQSPGTLSASPGERVTLSCRASSSVSYMYWYQQKPGQAPRPWIYATSNLASGVPDRFSGSGSGTDFTLTISRLEPEDFAVYYCQQWSGNPRTFGGGTKLEIK

SEQ ID NO: 1269 - protein, 106 aa, synthetic construct
EIVLTQSPGTLSASPGERVTMSCRASSSVSYMYWYQQKPGQAPRLLIYATSNLASGVPDRFSGSGSGTDFTLTISRVEPEDFAVYYCQQWSGNPRTFGGGTKLEIK

SEQ ID NO: 1270 - protein, 106 aa, synthetic construct
EIVLTQSPGTLSLSPGERVTMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 1271 - protein, 106 aa, synthetic construct
EIVLTQSPGTLSLSPGERATLSCRASSSVSYMYWYQQKPGQAPRLLIYATSNLASGVPDRFSGSGSGTDYTLTISRLEPEDFAVYYCQQWSGNPRTFGGGTKLEIK

SEQ ID NO: 1272 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWDGNPRTFGGGTKLEIK

SEQ ID NO: 1273 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWHGNPRTFGGGTKLEIK

SEQ ID NO: 1274 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWNGNPRTFGGGTKLEIK

SEQ ID NO: 1275 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWQGNPRTFGGGTKLEIK

SEQ ID NO: 1276 - protein, 106 aa, synthetic construct
  VARIANT 88: note = Q or N
  VARIANT 91: note = D, E, H, N, Q, or S
  VARIANT 92: note = A or G
  VARIANT 93: note = D, F, K, N, R, S, or T
EIVLTQSPGTLSLSPGERATLSCRASSSVSYMYWYQQKPGQAPRLLIYATSNLASGIPDRFSGSGSGTDFTLTISRLEPEDFAVYYCXQWXXXPRTFGGGTKLEIK

SEQ ID NO: 1277 - protein, 106 aa, synthetic construct
EIVLTQSPGTLSLSPGERATLSCGASSSVSYMYWYQQKPGQAPRLLIYATSNLASGIPDRFSGSGSGTDFTLTISRLEPEDFAVYYCQQWEGNPRTFGGGTKLEIK

SEQ ID NO: 1278 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCSQWSGNPRTFGGGTKLEIK

SEQ ID NO: 1279 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRSFGGGTKLEIK

SEQ ID NO: 1280 - protein, 113 aa, synthetic construct
DIVMTQSPDSLAVSLGERATINCKSSQSILYSSNNKNYLAWYQQKPGQPPKLLIYWASTRESGVPDRFSGSGSGTDFTLTISSLQAEDVSVYYCQQYYSTPFTFGPGTKVDIK

SEQ ID NO: 1281 - protein, 112 aa, synthetic construct
DIMLTQTPLTSPVTLGQPASISCKSSQSLVHSDGNTYLSWLQQRPGQPPRLLFYKISNRFSGVPDRFSGSGAGTDFTLKISRVEAEDVGVYYCMQATQFPLTFGGGTKVEIK

SEQ ID NO: 1282 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 1283 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1284 - protein, 107 aa, synthetic construct [sequence omitted]

SEQ ID NO: 1285 - protein, 106 aa, synthetic construct
DIQLTQSPSFLSASVGDRVTITCSASSSVNYMHWYQQKPGKAPKLLIYSTSNLASGVPSRFSGSGSGTEFTLTISSLQPEDFATYYCHQWNNYGTFGQGTKVEIKR

SEQ ID NO: 1286 - protein, 113 aa, synthetic construct
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGGGTKVEIKR

SEQ ID NO: 1287 - protein, 113 aa, synthetic construct
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGQGTKVEIKR

SEQ ID NO: 1288 - protein, 113 aa, synthetic construct
DVVMTQTPLSLPVTPGEPASISCKSSQNIVHSDGNTYLEWYLQKPGQSPQLLIYKVSNRFSGVPDRFSGSGSGTDFTLKISRVEAEDLGVYYCFQGSHVPLTFGGGTKVEIKR

SEQ ID NO: 1289 - protein, 113 aa, synthetic construct
DVVMTQTPLSLPVSLGDQASISCKSSQNIVHSDGNTYLEWYLQKPGQSPKVLIYKVSNRFSGVPDRFSGSGSGTDFTLKISRVEAEDLGVYYCFQGSHVPLTFGGGTKVEIKR

SEQ ID NO: 1290 - protein, 113 aa, synthetic construct
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGGGTKVEIKR

SEQ ID NO: 1291 - protein, 113 aa, synthetic construct
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGQGTKVEIKR

SEQ ID NO: 1292 - protein, 113 aa, synthetic construct
DVVMTQTPLSLPVTPGEPASISCRSSQSIVHSNGNTYLDWYLQKPGQSPQLLIYKVSNRFSGVPDRFSGSGSGTDFTLKISRVEAEDLGVYYCFQGSHVPLTFGGGTKVEIKR

SEQ ID NO: 1293 - protein, 113 aa, synthetic construct
DVVMTQTPLSLPVSLGDQASISCRSSQSIVHSNGNTYLDWYLQKPGQSPKVLIYKVSNRFSGVPDRFSGSGSGTDFTLKINRVEAEDLGVYFCFQGSHVPLTFGGGTKLEIKR

SEQ ID NO: 1294 - protein, 107 aa, synthetic construct
DIQMNQSPSSLSASLGDTITITCHASQNINVLLSWYQQKPGNIPKLLIYKASNLHTGVPSRFSGSGSGTGFTFTISSLQPEDIATYYCQQGQSYPYTFGGGTKLEIK

SEQ ID NO: 1295 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 1296 - protein, 107 aa, synthetic construct
DIQMTQSPSSLSASVGDRVTITCQASQNINVLLNWYQQKPGKAPKLLIYKASNLHTGVPSRFSGSGSGTDFTFTISSLQPEDIATYYCQQGQSYPYTFGQGTKLEIK

SEQ ID NO: 1297 - protein, 107 aa, synthetic construct
DIQMNQSPSSLSASVGDRVTITCQASQNINVLLSWYQQKPGKAPKLLIYKASNLHTGVPSRFSGSGSGTDFTFTISSLQPEDIATYYCQQGQSYPYTFGQGTKLEIK

SEQ ID NO: 1298 - protein, 111 aa, synthetic construct
  VARIANT 32: note = Any amino acid
  VARIANT 33: note = Any amino acid
  VARIANT 36: note = Any amino acid
  VARIANT 93: note = Any amino acid
QSVLTQPPSVSGAPGQRVTISCTSSSSDIGAXXGVXWYQQLPGTAPKLLIEGYYNRPSGVPDRFSGSKSGTSASLTITGLLPEDEGDYYCQSXDGTLSALFGGGTKLTVLG

SEQ ID NO: 1299 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 1300 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 1301 - protein, 118 aa, synthetic construct
  VARIANT 1: note = A, R, N, D, C, Q, E, G, H, I, L, K, M, F, P, S, T, W, Y, or V
  VARIANT 38: note = A, R, N, D, C, Q, E, G, H, I, L, K, M, F, P, S, T, W, Y, or V
  VARIANT 48: note = A, R, N, D, C, Q, E, G, H, I, L, K, M, F, P, S, T, W, Y, or V
  VARIANT 68: note = A, R, N, D, C, Q, E, G, H, I, L, K, M, F, P, S, T, W, Y, or V
  VARIANT 70: note = A, R, N, D, C, Q, E, G, H, I, L, K, M, F, P, S, T, W, Y, or V
  VARIANT 72: note = A, R, N, D, C, Q, E, G, H, I, L, K, M, F, P, S, T, W, Y, or V
  VARIANT 79: note = A, R, N, D, C, Q, E, G, H, I, L, K, M, F, P, S, T, W, Y, or V
  VARIANT 81: note = A, R, N, D, C, Q, E, G, H, I, L, K, M, F, P, S, T, W, Y, or V
  VARIANT 26..35: note = This region may encompass one of the following sequences: GFDIQDTYMH or DTYMH or KYDIN or GFEIQDTYMH or GFDPQDTYMH or GFDVQDTYMH or GFDIGDTYMH or GFDISDTYMH or GFDIVDTYMH or GFDIQDAYMH or GFDIQDSYMH or GFDIQDTFMH or GFDIQDTYIH or GFDLQDTYMH or GFDIQDTYLH or GFDIGDTFIH or GFDIGDTFMH or GFDIGDTYIH or GFDIQDTFIH or GFDIQDTFMH or GFDIQDTYIH or GFDISDTFIH or GFDISDTFMH or GFDISDTYIH or GFDISDTYMH or GFDIVDTFIH or GFDIVDTFMH or GFDIVDTYIH or
  VARIANT 26..35: note = CONT. FROM ABOVE: GFDPGDTFIH or GFDPGDTFMH or GFDPGDTYIH or GFDPGDTYMH or GFDPQDTFIH or GFDPQDTFMH or GFDPQDTYIH or GFDPQDTYMH or GFDPSDTFIH or GFDPSDTFMH or GFDPSDTYIH or GFDPSDTYMH or GFDPVDTFIH or GFDPVDTFMH or GFDPVDTYIH or GFDPVDTYMH or GFDVGDTFIH or GFDVGDTFMH or GFDVGDTYIH or GFDVGDTYMH or GFDVQDTFIH or GFDVQDTFMH or GFDVQDTYIH or GFDVSDTFIH or GFDVSDTFMH or GFDVSDTYIH or GFDVSDTYMH or GFDVVDTFIH or GFDVVDTFMH or GFDVVDTYIH or GFDVVDTYMH or GFEIGDTFIH or GFEIGDTFMH or GFEIGDTYIH or GFEIGDTYMH or GFEIQDTFIH or GFEIQDTFMH or GFEIQDTYIH or GFEIQDTYMH or GFEISDTFIH or GFEISDTFMH or GFEISDTYIH or GFEISDTYMH or GFEIVDTFIH or GFEIVDTFMH or GFEIVDTYIH or GFEIVDTYMH or GFEPGDTFIH or GFEPGDTFMH or GFEPGDTYIH or GFEPGDTYMH or GFEPQDTFIH or GFEPQDTFMH or
  VARIANT 26..35: note = CONT. FROM ABOVE: GFEPQDTYIH or GFEPQDTYMH or GFEPSDTFIH or GFEPSDTFMH or GFEPSDTYIH or GFEPSDTYMH or GFEPVDTFIH or GFEPVDTFMH or GFEPVDTYIH or GFEPVDTYMH or GFEVGDTFIH or GFEVGDTFMH or GFEVGDTYIH or GFEVGDTYMH or GFEVQDTFIH or GFEVQDTFMH or GFEVQDTYIH or GFEVQDTYMH or GFEVSDTFIH or GFEVSDTFMH or GFEVSDTYIH or GFEVSDTYMH or GFEVVDTFIH or GFEVVDTFMH or GFEVVDTYIH or GFEVVDTYMH or GFX1X2X3DX4X5X6H (wherein X1 = D or E, X2 = I, L, P, or V, X3 = G, Q, S, or V, X4 = A, S, or T, X5 = F or Y, and X6 = I, L, or M) or GFX1X2X3DTX4X5H (wherein X1 = D, or E, X2 = I, P, or V, X3 = G, Q, S, or V, X4 = F or Y, and X5 = I or M) or GFTFSSYW or GGSFTGFY or GYX1FX2X3YGIS (wherein X1 = P, S, D, Q, or N, X2 = T or R, and X3 = N, T, Y, or H) or GYDFTYYGIS or SRSYYWG or TSNMGVV or LYGMN or NYGMN or GYTFTSSWMH or GYTFTSYDIN or SYFWS or GYYWN or GFTFSTYG or TYGMS
  VARIANT 50..66: note = This region may encompass one of the following sequences: RIDPASGHTKYDPKFQV or RIEPASGHIKYDPKFQG or RIDPASGHIKYDPKFQG or RIEPASGHIKYDPKFQV or WIFPGDGRTDYNEKFKG or RLDPASGHTK or RIEPASGHTK or RIDPESGHTK or RIDPASGHTK or RIDPAGGHTK or RIDPASAHTK or RIDPASGHIK or RIDPASGHLK or RIDPASGHVK or YDPKFQV or IDPKFQV or LDPKFQV or MDPKFQV or SDPKFQV or TDPKFQV or VDPKFQV or YIPKFQV or YNPKFQV or YRPKFQV or YSPKFQV or YDPKFRV or YDPKFQA or YDPKFQD or YDPKFQE or YDPKFQG or YDPKFQH or YDPKFQK or YDPKFQL or YDPKFQM or YDPKFQN or YDPKFQP or YDPKFQR or YDPKFQS or YDPKFQT or RIEPASGHIKYDPKFQG or RIEPASGHIKYSPKFQG or RIEPASGHVKYSPKFQV or RIEPASGHVKYDPKFQT or RIDPASGHIKYDPKFQK or RIDPASGHVKIDPKFQV or RIDPASGHLKYDPKFQV or
  VARIANT 50..66: note = CONT. FROM ABOVE: RIDPASGHLKYDPKFQR or RIDPASGHLKYDPKFQN or RIEPASGHLKYDPKFQE or PASGH or RIDPASGHTKYDPKFQV or RIDPASGHLKYDPKFQG or RIDPASGHTKYDPKFQG or RIDPASGHSKYDPKFQV or RIDPASGHYKYDPKFQV or RX1X2PX3X4X5HX6KX7X8PKFX9X10 (wherein X1 = I or L, X2 = D or E, X3 = A or E, X4 = G or S, X5 = A or G, X6 = I, L, T, or V, X7 = I, L, M, S, T, V, or Y, X8 = D, I, N, R, or S, X9 = Q or R, and X10 = A, D, E, G, H, K, L, M, N, P, R, S, T, or V) or IKEDGSEK or INHRGNT or WISX1YNGX2X3X4YAX5X6X7QG (wherein X1 = T, P, S, or A, X2 = N, G, V, K, or A, X3 = T or K, X4 = H or N, X5 = Q or R, X6 = K or M, and X7 = L or H) or WISTYNGNTHYARMLQG or SIYYNGRTYYNPSLKS or HILWDDREYSNPALKS or WINTYTGEPTYADDFKG or IHPNSGGT or WLNPNSGXTG (wherein X = N or Y) or YIYYSGNTKYNPSLKS or EINHAGNTNYNPSLKS or ISGTGRTT or WMNTYSGVTTYADDFKG
  VARIANT 99..107: note = This region may encompass one of the following sequences: SGGLPDV or ARSGGLPDV or SGGLPDW or ARSGGLPDW or YGPAMDY or LGGLPDV or SAGLPDV or SGGAPDV or SGGMPDV or SGGLPEV or SGGLPDK or SGGLPDM or SGGLPDQ or SGGLPDR or SGGLPDS or SGGLPDT or SGGLPDH or SGGLPDF or SGGSPDV or ARSGGLPDM or ARSGGLPDK or SGGLPD or ARSGGLPDF or ARSGGLPDL or SGGLPDE or SGGLPDI or SGGLPDK or SGGLPDL or SGGLPDM or SGGLPDQ or SGGLPDT or SGGLPDW or SGGLPDY or SGGMPDE or SGGMPDI or SGGMPDK or SGGMPDL or SGGMPDM or SGGMPDQ or SGGMPDT or SGGMPDV or SGGMPDW or SGGMPDY or X1X2GX3PX4X5 (wherein X1 = L or S, X2 = A or G, X3 = A, L, or M, X4 = D or E, and X5 = K, M, Q, R, S, T, V, or W) or SGGX1PDX2 (wherein X1 = L or M and X2 = E, I, K, L, M, Q, T, V, W, or Y)
XVQLVQSGAEVKKPGASVKVSCKASXXXXXXXXXXWVXQRPGQGLEWXGXXXXXXXXXXXXXXXXXRXTXTXDTSTSTXYXELSSLRSEDTAVYYCARXXXXXXXXXWGQGTTVTVSS

SEQ ID NO: 1302 - protein, 116 aa, synthetic construct
  VARIANT 1: note = A, R, N, D, C, Q, E, G, H, I, L, K, M, F, P, S, T, W, Y, or V
  VARIANT 38: note = A, R, N, D, C, Q, E, G, H, I, L, K, M, F, P, S, T, W, Y, or V
  VARIANT 48: note = A, R, N, D, C, Q, E, G, H, I, L, K, M, F, P, S, T, W, Y, or V
  VARIANT 68: note = A, R, N, D, C, Q, E, G, H, I, L, K, M, F, P, S, T, W, Y, or V
  VARIANT 70: note = A, R, N, D, C, Q, E, G, H, I, L, K, M, F, P, S, T, W, Y, or V
  VARIANT 72: note = A, R, N, D, C, Q, E, G, H, I, L, K, M, F, P, S, T, W, Y, or V
  VARIANT 79: note = A, R, N, D, C, Q, E, G, H, I, L, K, M, F, P, S, T, W, Y, or V
  VARIANT 81: note = A, R, N, D, C, Q, E, G, H, I, L, K, M, F, P, S, T, W, Y, or V
  VARIANT 26..35: note = This region may encompass one of the following sequences: GFDIQDTYMH or DTYMH or KYDIN or GFEIQDTYMH or GFDPQDTYMH or GFDVQDTYMH or GFDIGDTYMH or GFDISDTYMH or GFDIVDTYMH or GFDIQDAYMH or GFDIQDSYMH or GFDIQDTFMH or GFDIQDTYIH or GFDLQDTYMH or GFDIQDTYLH or GFDIGDTFIH or GFDIGDTFMH or GFDIGDTYIH or GFDIQDTFIH or GFDIQDTFMH or GFDIQDTYIH or GFDISDTFIH or GFDISDTFMH or GFDISDTYIH or GFDISDTYMH or GFDIVDTFIH or GFDIVDTFMH or GFDIVDTYIH or
  VARIANT 26..35: note = CONT. FROM ABOVE: GFDPGDTFIH or GFDPGDTFMH or GFDPGDTYIH or GFDPGDTYMH or GFDPQDTFIH or GFDPQDTFMH or GFDPQDTYIH or GFDPQDTYMH or GFDPSDTFIH or GFDPSDTFMH or GFDPSDTYIH or GFDPSDTYMH or GFDPVDTFIH or GFDPVDTFMH or GFDPVDTYIH or GFDPVDTYMH or GFDVGDTFIH or GFDVGDTFMH or GFDVGDTYIH or GFDVGDTYMH or GFDVQDTFIH or GFDVQDTFMH or GFDVQDTYIH or GFDVSDTFIH or GFDVSDTFMH or GFDVSDTYIH or GFDVSDTYMH or GFDVVDTFIH or GFDVVDTFMH or GFDVVDTYIH or GFDVVDTYMH or GFEIGDTFIH or GFEIGDTFMH or GFEIGDTYIH or GFEIGDTYMH or GFEIQDTFIH or GFEIQDTFMH or GFEIQDTYIH or GFEIQDTYMH or GFEISDTFIH or GFEISDTFMH or GFEISDTYIH or GFEISDTYMH or GFEIVDTFIH or GFEIVDTFMH or GFEIVDTYIH or GFEIVDTYMH or GFEPGDTFIH or GFEPGDTFMH or GFEPGDTYIH or GFEPGDTYMH or GFEPQDTFIH or GFEPQDTFMH or
  VARIANT 26..35: note = CONT. FROM ABOVE: GFEPQDTYIH or GFEPQDTYMH or GFEPSDTFIH or GFEPSDTFMH or GFEPSDTYIH or GFEPSDTYMH or GFEPVDTFIH or GFEPVDTFMH or GFEPVDTYIH or GFEPVDTYMH or GFEVGDTFIH or GFEVGDTFMH or GFEVGDTYIH or GFEVGDTYMH or GFEVQDTFIH or GFEVQDTFMH or GFEVQDTYIH or GFEVQDTYMH or GFEVSDTFIH or GFEVSDTFMH or GFEVSDTYIH or GFEVSDTYMH or GFEVVDTFIH or GFEVVDTFMH or GFEVVDTYIH or GFEVVDTYMH or GFX1X2X3DX4X5X6H (wherein X1 = D or E, X2 = I, L, P, or V, X3 = G, Q, S, or V, X4 = A, S, or T, X5 = F or Y, and X6 = I, L, or M) or GFX1X2X3DTX4X5H (wherein X1 = D, or E, X2 = I, P, or V, X3 = G, Q, S, or V, X4 = F or Y, and X5 = I or M) or GFTFSSYW or GGSFTGFY or GYX1FX2X3YGIS (wherein X1 = P, S, D, Q, or N, X2 = T or R, and X3 = N, T, Y, or H) or GYDFTYYGIS or SRSYYWG or TSNMGVV or LYGMN or NYGMN or GYTFTSSWMH or GYTFTSYDIN or SYFWS or GYYWN or GFTFSTYG or TYGMS
  VARIANT 50..66: note = This region may encompass one of the following sequences: RIDPASGHTKYDPKFQV or RIEPASGHIKYDPKFQG or RIDPASGHIKYDPKFQG or RIEPASGHIKYDPKFQV or WIFPGDGRTDYNEKFKG or RLDPASGHTK or RIEPASGHTK or RIDPESGHTK or RIDPASGHTK or RIDPAGGHTK or RIDPASAHTK or RIDPASGHIK or RIDPASGHLK or RIDPASGHVK or YDPKFQV or IDPKFQV or LDPKFQV or MDPKFQV or SDPKFQV or TDPKFQV or VDPKFQV or YIPKFQV or YNPKFQV or YRPKFQV or YSPKFQV or YDPKFRV or YDPKFQA or YDPKFQD or YDPKFQE or YDPKFQG or YDPKFQH or YDPKFQK or YDPKFQL or YDPKFQM or YDPKFQN or YDPKFQP or YDPKFQR or YDPKFQS or YDPKFQT or RIEPASGHIKYDPKFQG or RIEPASGHIKYSPKFQG or RIEPASGHVKYSPKFQV or RIEPASGHVKYDPKFQT or RIDPASGHIKYDPKFQK or RIDPASGHVKIDPKFQV or RIDPASGHLKYDPKFQV or
  VARIANT 50..66: note = CONT. FROM ABOVE: RIDPASGHLKYDPKFQR or RIDPASGHLKYDPKFQN or RIEPASGHLKYDPKFQE or PASGH or RIDPASGHTKYDPKFQV or RIDPASGHLKYDPKFQG or RIDPASGHTKYDPKFQG or RIDPASGHSKYDPKFQV or RIDPASGHYKYDPKFQV or RX1X2PX3X4X5HX6KX7X8PKFX9X10 (wherein X1 = I or L, X2 = D or E, X3 = A or E, X4 = G or S, X5 = A or G, X6 = I, L, T, or V, X7 = I, L, M, S, T, V, or Y, X8 = D, I, N, R, or S, X9 = Q or R, and X10 = A, D, E, G, H, K, L, M, N, P, R, S, T, or V) or IKEDGSEK or INHRGNT or WISX1YNGX2X3X4YAX5X6X7QG (wherein X1 = T, P, S, or A, X2 = N, G, V, K, or A, X3 = T or K, X4 = H or N, X5 = Q or R, X6 = K or M, and X7 = L or H) or WISTYNGNTHYARMLQG or SIYYNGRTYYNPSLKS or HILWDDREYSNPALKS or WINTYTGEPTYADDFKG or IHPNSGGT or WLNPNSGXTG (wherein X = N or Y) or YIYYSGNTKYNPSLKS or EINHAGNTNYNPSLKS or ISGTGRTT or WMNTYSGVTTYADDFKG
  VARIANT 97..105: note = This region may encompass one of the following sequences: SGGLPDV or ARSGGLPDV or SGGLPDW or ARSGGLPDW or YGPAMDY or LGGLPDV or SAGLPDV or SGGAPDV or SGGMPDV or SGGLPEV or SGGLPDK or SGGLPDM or SGGLPDQ or SGGLPDR or SGGLPDS or SGGLPDT or SGGLPDH or SGGLPDF or SGGSPDV or ARSGGLPDM or ARSGGLPDK or SGGLPD or ARSGGLPDF or ARSGGLPDL or SGGLPDE or SGGLPDI or SGGLPDK or SGGLPDL or SGGLPDM or SGGLPDQ or SGGLPDT or SGGLPDW or SGGLPDY or SGGMPDE or SGGMPDI or SGGMPDK or SGGMPDL or SGGMPDM or SGGMPDQ or SGGMPDT or SGGMPDV or SGGMPDW or SGGMPDY or X1X2GX3PX4X5 (wherein X1 = L or S, X2 = A or G, X3 = A, L, or M, X4 = D or E, and X5 = K, M, Q, R, S, T, V, or W) or SGGX1PDX2 (wherein X1 = L or M and X2 = E, I, K, L, M, Q, T, V, W, or Y)
XVQLVQSGAEVKKPGASVKVSCKASXXXXXXXXXXWVXQRPGQGLEWXGXXXXXXXXXXXXXXXXXRXTXTXDTSTSTXYXELSSLRSEDTAVYYCXXXXXXXXXWGQGTTVTVSS

SEQ ID NO: 1303 - protein, 6 aa, synthetic construct
HHHHHH

SEQ ID NO: 1304 - protein, 111 aa, synthetic construct
QSVLTQPPSVSGAPGQRVTISCTSSSSDIGAALGVHWYQQLPGTAPKLLIEGYYNRPSGVPDRFSGSKSGTSASLTITGLLPEDEGDYYCQSWDGTLSALFGGGTKLTVLG

SEQ ID NO: 1305 - protein, 111 aa, synthetic construct
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSWDGTLSALFGGGTKLTVLG

SEQ ID NO: 1306 - protein, 111 aa, synthetic construct
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSWDGTLSALFGGGTKLTVLG

SEQ ID NO: 1307 - protein, 111 aa, synthetic construct
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSWDGTLSALFGGGTKLTVLG

SEQ ID NO: 1308 - protein, 111 aa, synthetic construct; identical to SEQ ID NO: 1299

SEQ ID NO: 1309 - protein, 111 aa, synthetic construct; identical to SEQ ID NO: 1305

SEQ ID NO: 1310 - protein, 111 aa, synthetic construct; identical to SEQ ID NO: 1306

SEQ ID NO: 1311 - protein, 111 aa, synthetic construct; identical to SEQ ID NO: 1307

SEQ ID NO: 1312 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSFDGTLSALFGGGTKLTVLG

SEQ ID NO: 1313 - protein, 107 aa, synthetic construct
DIQMTQSPSSLSASVGDRVTITCRASQSINNYLNWYQQRPGKAPKLLIYAASSLQSGVPSRFSGSGSGTDFTLTISSLQPGDFATYYCQQSYSTPRTFGQGTKLEIK

SEQ ID NO: 1314 - protein, 107 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSPTFGQGTRLEIK

SEQ ID NO: 1315 - protein, 106 aa, synthetic construct
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 1316 - protein, 112 aa, synthetic construct
DVLMTQTPLSLPVSLGDQASISCRSSQNIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 1317 - protein, 14 aa, synthetic construct
WVRQAPGQGLEWMG

SEQ ID NO: 1318 - protein, 30 aa, synthetic construct
RVTMTRDTSTSTVYMELSSLRSEDTAVYYC

SEQ ID NO: 1319 - protein, 30 aa, synthetic construct
RATITTDTSASTAYLQLSSLRSEDTAVYYC

SEQ ID NO: 1320 - protein, 30 aa, synthetic construct
RVTITRDTSASTVYMELSSLRSEDTAVYYC

SEQ ID NO: 1321 - protein, 30 aa, synthetic construct
RVTITRDTSASTAYMELSSLRSEDTAVYYC

SEQ ID NO: 1322 - protein, 32 aa, synthetic construct
RVTMTRDTSTSTVYMELSSLRSEDTAVYYCSR

SEQ ID NO: 1323 - protein, 32 aa, synthetic construct
RVTMTRDTSTSTVYMELSSLRSEDTAVYYCAR

SEQ ID NO: 1324 - protein, 15 aa, synthetic construct
WYQQKPGQAPRLLIY

SEQ ID NO: 1325 - protein, 32 aa, synthetic construct
GVPDRFSGSGSGTDYTLTISRLEPEDFAVYYC

SEQ ID NO: 1326-2049 - skipped (724 entries)

SEQ ID NO: 2050 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2051 - protein, 192 aa, Homo sapiens
MQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2052 - protein, 174 aa, Homo sapiens
MRRFLSKVYSFPMRKLILFLVFPVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2053-2300 - skipped (248 entries)

SEQ ID NO: 2301 - DNA, 19 bp, synthetic construct
  source 1..19: mol_type = other DNA
tgcttctggaagtgaaagt

SEQ ID NO: 2302 - DNA, 21 bp, synthetic construct
  source 1..21: mol_type = other DNA
tgaggttcaaaatgacagagg

SEQ ID NO: 2303 - DNA, 12 bp, synthetic construct
  source 1..12: mol_type = other DNA
  modified_base 3: mod_base = OTHER; note = Locked nucleic acid
  modified_base 4: mod_base = OTHER; note = Locked nucleic acid
  modified_base 6: mod_base = OTHER; note = Locked nucleic acid
  modified_base 7: mod_base = OTHER; note = Locked nucleic acid
  modified_base 8: mod_base = OTHER; note = Locked nucleic acid
tgcagattggga

SEQ ID NO: 2304 - DNA, 12 bp, synthetic construct
  source 1..12: mol_type = other DNA
  modified_base 3: mod_base = OTHER; note = Locked nucleic acid
  modified_base 4: mod_base = OTHER; note = Locked nucleic acid
  modified_base 7: mod_base = OTHER; note = Locked nucleic acid
tgcaggttggga

SEQ ID NO: 2305-3000 - skipped (696 entries)

SEQ ID NO: 3001 - protein, 10 aa, synthetic construct
GFDIQDTYMH

SEQ ID NO: 3002 - protein, 17 aa, synthetic construct
RIDPASGHTKYDPKFQV

SEQ ID NO: 3003 - protein, 17 aa, synthetic construct
RIEPASGHIKYDPKFQG

SEQ ID NO: 3004 - skipped

SEQ ID NO: 3005 - protein, 17 aa, synthetic construct
RIEPASGHIKYDPKFQV

SEQ ID NO: 3006 - protein, 7 aa, synthetic construct
SGGLPDV

SEQ ID NO: 3007 - skipped

SEQ ID NO: 3008 - protein, 7 aa, synthetic construct
SGGLPDW

SEQ ID NO: 3009 - skipped

SEQ ID NO: 3010 - protein, 10 aa, synthetic construct
RASSSVSYMY

SEQ ID NO: 3011 - protein, 7 aa, synthetic construct
ATSNLAS

SEQ ID NO: 3012 - protein, 9 aa, synthetic construct
QQWEGNPRT

SEQ ID NO: 3013 - protein, 9 aa, synthetic construct
QQWKGNPRT

SEQ ID NO: 3014-3115 - skipped (102 entries)

SEQ ID NO: 3116 - protein, 116 aa, synthetic construct; identical to SEQ ID NO: 116

SEQ ID NO: 3117-3120 - skipped (4 entries)

SEQ ID NO: 3121 - protein, 116 aa, synthetic construct; identical to SEQ ID NO: 121

SEQ ID NO: 3122 - protein, 116 aa, synthetic construct; identical to SEQ ID NO: 122

SEQ ID NO: 3123 - protein, 116 aa, synthetic construct; identical to SEQ ID NO: 123

SEQ ID NO: 3124 - protein, 116 aa, synthetic construct; identical to SEQ ID NO: 124

SEQ ID NO: 3125 - protein, 116 aa, synthetic construct; identical to SEQ ID NO: 125

SEQ ID NO: 3126-3127 - skipped (2 entries)

SEQ ID NO: 3128 - protein, 116 aa, synthetic construct; identical to SEQ ID NO: 128

SEQ ID NO: 3129 - protein, 116 aa, synthetic construct; identical to SEQ ID NO: 129

SEQ ID NO: 3130 - skipped

SEQ ID NO: 3131 - protein, 116 aa, synthetic construct; identical to SEQ ID NO: 131

SEQ ID NO: 3132 - skipped

SEQ ID NO: 3133 - protein, 116 aa, synthetic construct; identical to SEQ ID NO: 133

SEQ ID NO: 3134 - protein, 116 aa, synthetic construct; identical to SEQ ID NO: 134

SEQ ID NO: 3135 - skipped

SEQ ID NO: 3136 - protein, 116 aa, synthetic construct; identical to SEQ ID NO: 135

SEQ ID NO: 3137 - protein, 116 aa, synthetic construct; identical to SEQ ID NO: 137

SEQ ID NO: 3138 - protein, 116 aa, synthetic construct; identical to SEQ ID NO: 138

SEQ ID NO: 3139 - protein, 116 aa, synthetic construct; identical to SEQ ID NO: 139

SEQ ID NO: 3140-3150 - skipped (11 entries)

SEQ ID NO: 3151 - protein, 116 aa, synthetic construct; identical to SEQ ID NO: 151

SEQ ID NO: 3152 - protein, 116 aa, synthetic construct; identical to SEQ ID NO: 152

SEQ ID NO: 3153 - protein, 116 aa, synthetic construct; identical to SEQ ID NO: 153

SEQ ID NO: 3154 - protein, 116 aa, synthetic construct; identical to SEQ ID NO: 154

SEQ ID NO: 3155 - protein, 116 aa, synthetic construct; identical to SEQ ID NO: 155

SEQ ID NO: 3156 - protein, 116 aa, synthetic construct; identical to SEQ ID NO: 156

SEQ ID NO: 3157 - protein, 116 aa, synthetic construct; identical to SEQ ID NO: 157

SEQ ID NO: 3158 - protein, 116 aa, synthetic construct; identical to SEQ ID NO: 158

SEQ ID NO: 3159 - protein, 116 aa, synthetic construct; identical to SEQ ID NO: 159

SEQ ID NO: 3160 - protein, 116 aa, synthetic construct; identical to SEQ ID NO: 160

SEQ ID NO: 3161 - protein, 116 aa, synthetic construct; identical to SEQ ID NO: 161

SEQ ID NO: 3162 - protein, 116 aa, synthetic construct; identical to SEQ ID NO: 162

SEQ ID NO: 3163-3167 - skipped (5 entries)

SEQ ID NO: 3168 - protein, 116 aa, synthetic construct; identical to SEQ ID NO: 168

SEQ ID NO: 3169 - protein, 116 aa, synthetic construct; identical to SEQ ID NO: 169

SEQ ID NO: 3170-3201 - skipped (32 entries)

SEQ ID NO: 3202 - protein, 106 aa, synthetic construct; identical to SEQ ID NO: 202

SEQ ID NO: 3203 - skipped

SEQ ID NO: 3204 - protein, 106 aa, synthetic construct; identical to SEQ ID NO: 204

SEQ ID NO: 3205 - protein, 106 aa, synthetic construct; identical to SEQ ID NO: 205

SEQ ID NO: 3206 - protein, 106 aa, synthetic construct; identical to SEQ ID NO: 206

SEQ ID NO: 3207 - protein, 106 aa, synthetic construct; identical to SEQ ID NO: 207

SEQ ID NO: 3208 - protein, 106 aa, synthetic construct; identical to SEQ ID NO: 208

SEQ ID NO: 3209 - protein, 106 aa, synthetic construct; identical to SEQ ID NO: 209

SEQ ID NO: 3210-3215 - skipped (6 entries)

SEQ ID NO: 3216 - protein, 106 aa, synthetic construct; identical to SEQ ID NO: 216

SEQ ID NO: 3217 - protein, 106 aa, synthetic construct; identical to SEQ ID NO: 217

SEQ ID NO: 3218 - protein, 106 aa, synthetic construct; identical to SEQ ID NO: 218

SEQ ID NO: 3219 - protein, 106 aa, synthetic construct; identical to SEQ ID NO: 219

SEQ ID NO: 3220 - protein, 106 aa, synthetic construct; identical to SEQ ID NO: 220

SEQ ID NO: 3221-3318 - skipped (98 entries)

SEQ ID NO: 3319 - protein, 107 aa, synthetic construct [sequence omitted]

SEQ ID NO: 3320 - protein, 330 aa, synthetic construct [sequence omitted]